(19) United States
(12) Patent Application Publication
(10) Pub. No.: US 2023/0158157 A1
(43) Pub. Date: May 25, 2023

(54) POTENT AND SELECTIVE DEGRADERS OF ALK

(71) Applicant: DANA-FARBER CANCER INSTITUTE, INC., Boston, MA (US)

(72) Inventors: John M. Hatcher, Boston, MA (US); Nathanael S. Gray, Stanford, CA (US); Baishan Jiang, Watertown, MA (US); Tinghu Zhang, Brookline, MA (US); Jianwei Che, Sharon, MA (US); Yang Gao, Ashland, MA (US); Lyn Howard Jones, Winchester, MA (US)

(73) Assignee: DANA-FARBER CANCER INSTITUTE, INC., Boston, MA (US)

(21) Appl. No.: 17/801,709

(22) PCT Filed: Feb. 24, 2021
(86) PCT No.: PCT/US21/19424

§ 371 Date: Aug. 23, 2022

Related U.S. Application Data
(60) Provisional application No. 62/981,331, filed on Feb. 25, 2020.
(60) Provisional application No. 63/135,821, filed on Jan. 11, 2021.

(51) Int. Cl. A61K 47/55

(52) U.S. Cl. CPC A61K 47/55

(57) ABSTRACT

Disclosed are bispecific compounds (degraders) that target ALK or ALK and FAK for degradation. Also disclosed are pharmaceutical compositions containing the degraders and methods of using the bispecific compounds to treat diseases and disorders characterized or mediated by aberrant ALK or ALK and FAK activity.

### RELATED APPLICATIONS

This application is a national stage application, filed under 35 U.S.C. § 371, of International Application No. PCT/US2021/019424, filed Feb. 24, 2021, which claims the benefit of priority under 35 U.S.C. § 119(e) to U.S. Provisional Application No. 62/981,331, filed Feb. 25, 2020 and U.S. Provisional Application No. 63/135,821, filed Jan. 11, 2021, each of which is incorporated herein by reference in its entirety.

### BACKGROUND OF THE INVENTION

Anaplastic lymphoma kinase (ALK) is a receptor tyrosine kinase that was first identified in a chromosomal translocation associated with anaplastic large cell lymphoma (ALCL), a subtype of T-cell non-Hodgkin's lymphoma (Chiarle et al., Nat. Rev. Cancer 8(1):11-23 (2008)). Chromosomal translocations involving the kinase domain of ALK are seen in many cancers. In addition to ALCL, ALK fusion proteins are seen in diffuse large B-cell lymphoma (DLBCL), inflammatory myofibroblastic tumor (IMT), breast cancer, colorectal cancer, esophageal squamous cell cancer (ESCC), renal cell cancer (RCC), and non-small-cell lung cancer (NSCLC) (Roskoski, Pharmacol. Res. 68(1):68-94 (2013)). ALK fusion partners drive dimerization of the ALK kinase domain, leading to autophosphorylation, which in turn causes the kinase to become constitutively active (Bayliss et al., Cell. Mol. Life Sci. 73(6):1209-1224 (2016)). Oncogenic ALK may also be expressed due to point mutations as is seen in neuroblastoma (NB), where germline mutations in ALK have been documented to drive the majority of hereditary NB cases (George et al., Nature 455(7215):975-978 (2008); Mosse et al., Nature 455(7215):930-935 (2008)). Constitutively active oncogenic ALK signals through multiple pathways, including PI3K/AKT, RAS/ERK, and JAK/STAT3, which leads to enhanced cell proliferation and survival (Palmer et al., Biochem. J. 420(3):345-361 (2009)).

ALK-rearranged NSCLC represents ˜5% of all NSCLC and is a unique targetable molecular and clinical subset of NSCLC. Several studies have shown that NSCLC patients harboring ALK rearrangements are more likely to be non-smokers (Sasaki et al., Eur. J. Cancer 46(10):1773-1780 (2010); Mino-Kenudson et al., Clin. Cancer Res. 16(5):1561-1571 (2010)). Patients with tumors harboring such rearrangements are highly sensitive to ALK inhibitors (Arbour et al., Hematol. Oncol. Clin. North Am. 31(1):101-111 (2018)). Screening for ALK rearrangements is widely available throughout the United States and worldwide and is the standard of care for newly diagnosed advanced NSCLC patients (Camidge et al., Cancer 118(18):4486-4494 (2012); Kwak et al., New Engl. J. Med. 363(18):1693-1703 (2010)).

There are currently five FDA approved kinase inhibitors for the treatment of ALK-positive NSCLC, namely crizotinib, ceritinib (LDK378), alectinib, brigatinib and loratinib. ALK-positive tumors are highly sensitive to ALK inhibition, indicating that these tumors are addicted to ALK kinase activity. However despite initial dramatic responses of variable median duration (10.9 months for crizotinib, 25.7 months for alectinib), resistance to therapy typically develops (Peters et al., N. Engl. J. Med. 377:829-838 (2017); Soria et al., Lancet 389:917-929 (2017); Katayama et al., Sci. Trans. Med. 4(120):120ra17 (2012); Cooper et al., Ann. Pharmacother. 49:107-112 (2015); Sullivan et al., Ther. Adv. Med. Oncl. 8:32-47 (2016)).

Next-generation ALK inhibitors such as loratinib (approved by the FDA in November 2018 for use in the treatment of lung cancer) have been able to successfully target resistant tumors and have shown improvements in potency and overall response rates relative to approved inhibitors. However, resistance to these next-generation ALK inhibitors still arises in patients (Mologni et al., Transl. Lung Cancer Res. 4:5-7 (2015); Katayama et al., Clin. Cancer Res. 20:5686-5696 (2014); Qin et al., Targeted Oncology 12:709-718 (2017); Shaw et al., N. Engl. J. Med. 374:54-61 (2016)). The progression free survival period is currently at less than 12 months for patients that eventually acquire resistance (Mologni, Transl. Lung Cancer Res. 4(1):5-7 (2015); Katayama et al., Clin. Cancer Res. 20(22):5686-5696 (2014); Qin et al., Target. Oncol. 12(6):709-718 (2017); Shaw et al., New Engl. J. Med. 374(1):54-61 (2016)).

The three most prevalent resistance mechanisms are mutation in the ALK kinase domain, upregulation of ALK as a result of gene amplification or copy number gain, and/or activation of ALK-independent signal transduction pathways (Roskoski, Pharmacol. Res. 68(1):68-94 (2013)). Therapeutic strategies that target ALK employing novel mechanisms of action may provide ways to further delay the emergence of resistance mutations.

### SUMMARY OF THE INVENTION

A first aspect of the present invention is directed to a bispecific compound of formula (I),

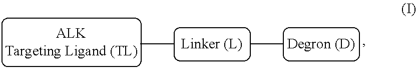

or a pharmaceutically acceptable salt or stereoisomer thereof, wherein the ALK targeting ligand is a brigatinib analog, a ceritinib analog, or a [6-{[(1S)-1-(5-fluoropyridin-2-yl)ethyl]amino}-1-(5-methyl-1H-pyrazol-3-yl)-1H-pyrrolo[2,3-b]pyridine analog.

Another aspect of the present invention is directed to a pharmaceutical composition containing a therapeutically effective amount of a bispecific compound of formula (I) or a pharmaceutically acceptable salt or stereoisomer thereof, and a pharmaceutically acceptable carrier.

In another aspect of the present invention, methods of making the bispecific compounds are provided.

A further aspect of the present invention is directed to a method of treating a disease or disorder involving (characterized or mediated by) aberrant ALK or aberrant ALK and aberrant focal adhesion kinase (FAK) activity, that includes administering a therapeutically effective amount of a bispecific compound of formula (I) or a pharmaceutically acceptable salt or stereoisomer thereof, to a subject in need thereof.

Without intending to be bound by any particular theory of operation, bispecific compounds of formula I (also referred to herein as PROTACs or degraders) are believed to promote the degradation of ALK or ALK and FAK via cells' Ubiquitin/Proteasome System, whose function is to routinely identify and remove damaged proteins. After destruction of an ALK or ALK and FAK molecules, the degrader is released and continues to be active. Therefore, by engaging and exploiting the body's own natural protein disposal system, bispecific compounds of the present invention may represent a potential improvement over current small molecule inhibitors of ALK and FAK. Therefore, effective intracellular concentrations of the degraders may be significantly lower than for small molecule ALK and FAK inhibitors.

Accordingly, bispecific compounds of the present invention may offer at least one additional advantage including improved pharmacodynamics effects. The degradation of ALK or ALK and FAK may decrease tyrosine kinase inhibitor resistance imparted by intrinsic scaffolding functions of kinases and may also decrease the likelihood of de novo resistance mutations to the degraders since efficient degradation of ALK or ALK and FAK may be achieved with targeting ligands that have relatively less affinity to ALK or ALK and FAK compared to known ALK and FAK inhibitors. Collectively, present bispecific compounds may represent an advancement over known ALK and FAK inhibitors and may overcome one or more limitations regarding their use.

### BRIEF DESCRIPTION OF THE DRAWINGS

FIG. 1A is a Western Blot that shows the levels of pALK^{Tyr1507}, ALK and tubulin in WT EML4-ALK V3 Ba/F3 cells. Cells treated with DMSO, compound 96 or Alectinib at indicated concentrations (nM).

FIG. 1B is a Western Blot that shows the levels of pALK^{Tyr1507}, ALK and tubulin in G1202R EML4-ALK V3 Ba/F3 cells. Cells treated with DMSO, compound 96 or Alectinib at indicated concentrations (nM).

FIG. 1C is a Western Blot that shows the levels of pALK^{Tyr1507}, ALK and tubulin in WT EML4-ALK V3 Ba/F3 cells. Cells treated with DMSO, compound 96, negative control of 96 or Loratinib at indicated concentrations (nM).

FIG. 1D is a Western Blot that shows the levels of pALK^{Tyr1507}, ALK and tubulin in G1202R EML4-ALK V3 Ba/F3 cells. Cells treated with DMSO, compound 96, negative control of 96 or Loratinib at indicated concentrations (nM).

### DETAILED DESCRIPTION OF THE INVENTION

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present invention.

As used in the description and the appended claims, the singular forms “a” “an”, and “the” include plural referents unless the context clearly dictates otherwise. Therefore, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2%, or 1%) of the particular value modified by the term “about.”

The transitional term “comprising,” which is synonymous with “including,” “containing,” or “characterized by,” is inclusive or open-ended and does not exclude additional, unrecited elements or method steps. When used in the context of the number of heteroatoms in a heterocyclic structure, it means that the heterocyclic group that that minimum number of heteroatoms. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

With respect to compounds of the present invention, and to the extent the following terms are used herein to further describe them, the following definitions apply.

As used herein, the term “alkyl” refers to a saturated linear or branched-chain monovalent hydrocarbon radical. In one embodiment, the alkyl radical is a C₁-C₁₈ group. In other embodiments, the alkyl radical is a C₀-C₆, C₀-C₅, C₀-C₃, C₁-C₁₂, C₁-C₈, C₁-C₆, C₁-C₅, C₁-C₄ or C₁-C₃ group (wherein C₀ alkyl refers to a bond). Examples of alkyl groups include methyl, ethyl, 1-propyl, 2-propyl, i-propyl, 1-butyl, 2-methyl-1-propyl, 2-butyl, 2-methyl-2-propyl, 1-pentyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, heptyl, octyl, nonyl, decyl, undecyl and dodecyl. In some embodiments, an alkyl group is a C₁-C₃ alkyl group. In some embodiments, an alkyl group is a C₁-C₂ alkyl group, or a methyl group.

As used herein, the term “alkylene” refers to a straight or branched divalent hydrocarbon chain linking the rest of the molecule to a radical group, consisting solely of carbon and hydrogen, containing no unsaturation and having from one to 12 carbon atoms, for example, methylene, ethylene, propylene, n-butylene, and the like. The alkylene chain may be attached to the rest of the molecule through a single bond and to the radical group through a single bond. In some embodiments, the alkylene group contains one to 8 carbon atoms (C₁-C₈ alkylene). In other embodiments, an alkylene group contains one to 5 carbon atoms (C₁-C₅ alkylene). In other embodiments, an alkylene group contains one to 4 carbon atoms (C₁-C₄ alkylene). In other embodiments, an alkylene contains one to three carbon atoms (C₁-C₃ alkylene). In other embodiments, an alkylene group contains one to two carbon atoms (C₁-C₂ alkylene). In other embodiments, an alkylene group contains one carbon atom (C₁ alkylene).

As used herein, the term “alkenyl” refers to a linear or branched-chain monovalent hydrocarbon radical with at least one carbon-carbon double bond. An alkenyl includes radicals having “cis” and “trans” orientations, or alternatively, “E” and “Z” orientations. In one example, the alkenyl radical is a C₂-C₁₈ group. In other embodiments, the alkenyl radical is a C₂-C₁₂, C₂-C₁₀, C₂-C₈, C₂-C₆ or C₂-C₃ group. Examples include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl.

As used herein, the term “alkynyl” refers to a linear or branched monovalent hydrocarbon radical with at least one carbon-carbon triple bond. In one example, the alkynyl radical is a C₂-C₁₈ group. In other examples, the alkynyl radical is C₂-C₁₂, C₂-C₁₀, C₂-C₈, C₂-C₆ or C₂-C₃. Examples include ethynyl prop-1-ynyl, prop-2-ynyl, but-1-ynyl, but-2-ynyl and but-3-ynyl.

The terms “alkoxyl” or “alkoxy” as used herein refer to an alkyl group, as defined above, having an oxygen radical attached thereto, and which is the point of attachment. Representative alkoxyl groups include methoxy, ethoxy, propyloxy, tert-butoxy and the like. An “ether” is two hydrocarbyl groups covalently linked by an oxygen. Accordingly, the substituent of an alkyl that renders that alkyl an ether is or resembles an alkoxyl, such as can be represented by one of —O-alkyl, —O-alkenyl, and —O-alkynyl.

As used herein, the term “halogen” (or “halo” or “halide”) refers to fluorine, chlorine, bromine, or iodine.

As used herein, the term “cyclic group” broadly refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. Cyclic groups may have one or more (e.g., fused) ring systems. Therefore, for example, a cyclic group can contain one or more carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 20 carbon atoms, that is alone or part of a larger moiety (e.g., an alkcarbocyclic group). The term carbocyclyl includes mono-, bi-, tri-, fused, bridged, and spiro-ring systems, and combinations thereof. In one embodiment, carbocyclyl includes 3 to 15 carbon atoms (C₃-C₁₅). In one embodiment, carbocyclyl includes 3 to 12 carbon atoms (C₃-C₁₂). In another embodiment, carbocyclyl includes C₃-C₈, C₃-C₁₀ or C₅-C₁₀. In another embodiment, carbocyclyl, as a monocycle, includes C₃-C₈, C₃-C₆ or C₅-C₆. In some embodiments, carbocyclyl, as a bicycle, includes C₇-C₁₂. In another embodiment, carbocyclyl, as a spiro system, includes C₅-C₁₂. Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, phenyl, and cyclododecyl; bicyclic carbocyclyls having 7 to 12 ring atoms include [4,3], [4,4], [4,5], [5,5], [5,6] or [6,6] ring systems, such as for example bicyclo[2.2.1]heptane, bicyclo[2.2.2]octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocycyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

Therefore, the term carbocyclic also embraces carbocyclylalkyl groups which as used herein refer to a group of the formula —R^{c}-carbocyclyl where R^{c} is an alkylene chain. The term carbocyclic also embraces carbocyclylalkoxy groups which as used herein refer to a group bonded through an oxygen atom of the formula —O—R^{c}-carbocyclyl where R^{c} is an alkylene chain.

As used herein, the term “aryl” used alone or as part of a larger moiety (e.g., “aralkyl”, wherein the terminal carbon atom on the alkyl group is the point of attachment, e.g., a benzyl group), “aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. In some embodiments, the aralkoxy group is a benzoxy group. The term “aryl” may be used interchangeably with the term “aryl ring”. In one embodiment, aryl includes groups having 6-18 carbon atoms. In another embodiment, aryl includes groups having 6-10 carbon atoms. Examples of aryl groups include phenyl, naphthyl, anthracyl, biphenyl, phenanthrenyl, naphthacenyl, 1,2,3,4-tetrahydronaphthalenyl, 1H-indenyl, 2,3-dihydro-1H-indenyl, naphthyridinyl, and the like, which may be substituted or independently substituted by one or more substituents described herein. A particular aryl is phenyl. In some embodiments, an aryl group includes an aryl ring fused to one or more (e.g., 1, 2 or 3) different cyclic groups (e.g., carbocyclic rings or heterocyclic rings), where the radical or point of attachment is on the aryl ring.

Therefore, the term aryl embraces aralkyl groups (e.g., benzyl) which as disclosed above refer to a group of the formula —R^{c}-aryl where R^{c} is an alkylene chain such as methylene or ethylene. In some embodiments, the aralkyl group is an optionally substituted benzyl group. The term aryl also embraces aralkoxy groups which as used herein refer to a group bonded through an oxygen atom of the formula —O—R^{c}-aryl where R^{c} is an alkylene chain such as methylene or ethylene.

As used herein, the term “heterocyclyl” refers to a “carbocyclyl” that used alone or as part of a larger moiety, contains a saturated, partially unsaturated or aromatic ring system, wherein one or more (e.g., 1, 2, 3, or 4) carbon atoms have been replaced with a heteroatom (e.g., O, N, N(O), S, S(O), or S(O)₂). The term heterocyclyl includes mono-, bi-, tri-, fused, bridged, and spiro-ring systems, and combinations thereof. In some embodiments, a heterocyclyl refers to a 3 to 15 membered heterocyclyl ring system. In some embodiments, a heterocyclyl refers to a 3 to 12 membered heterocyclyl ring system. In some embodiments, a heterocyclyl refers to a saturated ring system, such as a 3 to 12 membered saturated heterocyclyl ring system. In some embodiments, a heterocyclyl refers to a heteroaryl ring system, such as a 5 to 14 membered heteroaryl ring system. The term heterocyclyl also includes C₃-C₈ heterocycloalkyl, which is a saturated or partially unsaturated mono-, bi-, or spiro-ring system containing 3-8 carbons and one or more (1, 2, 3 or 4) heteroatoms.

In some embodiments, a heterocyclyl group includes 3-12 ring atoms and includes monocycles, bicycles, tricycles and spiro ring systems, wherein the ring atoms are carbon, and one to 5 ring atoms is a heteroatom such as nitrogen, sulfur or oxygen. In some embodiments, heterocyclyl includes 3- to 7-membered monocycles having one or more heteroatoms selected from nitrogen, sulfur and oxygen. In some embodiments, heterocyclyl includes 4- to 6-membered monocycles having one or more heteroatoms selected from nitrogen, sulfur and oxygen. In some embodiments, heterocyclyl includes 3-membered monocycles. In some embodiments, heterocyclyl includes 4-membered monocycles. In some embodiments, heterocyclyl includes 5-6 membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, thiophenyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl, including thiazol-2-yl and thiazol-2-yl N-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Example 5-membered ring heterocyclyls containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Representative examples of benzo-fused 5-membered heterocyclyls are benzoxazol-2-yl, benzthiazol-2-yl and benzimidazol-2-yl. Example 6-membered heterocyclyls contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl. The pyridine N-oxides and pyridazine N-oxides and the pyridyl, pyrimid-2-yl, pyrimid-4-yl, pyridazinyl and the 1,3,4-triazin-2-yl groups, are yet other examples of heterocyclyl groups. In some embodiments, a heterocyclic group includes a heterocyclic ring fused to one or more (e.g., 1, 2 or 3) different cyclic groups (e.g., carbocyclic rings or heterocyclic rings), where the radical or point of attachment is on the heterocyclic ring, and in some embodiments wherein the point of attachment is a heteroatom contained in the heterocyclic ring.

Therefore, the term heterocyclic embraces N-heterocyclyl groups which as used herein refer to a heterocyclyl group containing at least one nitrogen and where the point of attachment of the heterocyclyl group to the rest of the molecule is through a nitrogen atom in the heterocyclyl group. Representative examples of N-heterocyclyl groups include 1-morpholinyl, 1-piperidinyl, 1-piperazinyl, 1-pyrrolidinyl, pyrazolidinyl, imidazolinyl and imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. Representative examples of C-heterocyclyl radicals include 2-morpholinyl, 2- or 3- or 4-piperidinyl, 2-piperazinyl, and 2- or 3-pyrrolidinyl. The term heterocyclic also embraces heterocyclylalkyl groups which as disclosed above refer to a group of the formula —R^{c}-heterocyclyl where R^{c} is an alkylene chain. The term heterocyclic also embraces heterocyclylalkoxy groups which as used herein refer to a radical bonded through an oxygen atom of the formula —O—R^{c}-heterocyclyl where R^{C} is an alkylene chain.

As used herein, the term “heteroaryl” used alone or as part of a larger moiety (e.g., “heteroarylalkyl” (also “heteroaralkyl”), or “heteroarylalkoxy” (also “heteroaralkoxy”), refers to a monocyclic, bicyclic or tricyclic ring system having 5 to 14 ring atoms, wherein at least one ring is aromatic and contains at least one heteroatom. In one embodiment, heteroaryl includes 5-6 membered monocyclic aromatic groups where one or more ring atoms is nitrogen, sulfur or oxygen. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridazinyl, triazinyl, tetrazinyl, tetrazolo[1,5-b]pyridazinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, 1,2,3-triazol-5-yl, and pyrid-2-yl N-oxide. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methylenedioxyphenyl, benzofuranyl, dibenzofuranyl, indazolyl, benzimidazolyl, benzodioxazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3(4H)-one. A heteroaryl group may be mono-, bi- or tri-cyclic. In some embodiments, a heteroaryl group includes a heteroaryl ring fused to one or more (e.g., 1, 2 or 3) different cyclic groups (e.g., carbocyclic rings or heterocyclic rings), where the radical or point of attachment is on the heteroaryl ring, and in some embodiments wherein the point of attachment is a heteroatom contained in the heterocyclic ring.

Therefore, the term heteroaryl embraces N-heteroaryl groups which as used herein refer to a heteroaryl group as defined above containing at least one nitrogen and where the point of attachment of the heteroaryl group to the rest of the molecule is through a nitrogen atom in the heteroaryl group. The term heteroaryl also embraces C-heteroaryl groups which as used herein refer to a heteroaryl group as defined above and where the point of attachment of the heteroaryl group to the rest of the molecule is through a carbon atom in the heteroaryl group. The term heteroaryl also embraces heteroarylalkyl groups which as disclosed above refer to a group of the formula —R^{c}-heteroaryl, wherein R^{c} is an alkylene chain as defined above. The term heteroaryl also embraces heteroaralkoxy (or heteroarylalkoxy) groups which as used herein refer to a group bonded through an oxygen atom of the formula —O—R^{c}-heteroaryl, where R^{c} is an alkylene group as defined above.

Unless stated otherwise, and to the extent not further defined for any particular group(s), any of the groups described herein may be substituted or unsubstituted. As used herein, the term “substituted” broadly refers to all permissible substituents with the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc. Representative substituents include halogens, hydroxyl groups, and any other organic groupings containing any number of carbon atoms, e.g., 1-14 carbon atoms, and which may include one or more (e.g., 1, 2, 3, or 4) heteroatoms such as oxygen, sulfur, and nitrogen grouped in a linear, branched, or cyclic structural format.

To the extent not disclosed otherwise for any particular group(s), representative examples of substituents may include alkyl, substituted alkyl (e.g., C₁-C₆, C₁-C₅, C₁-C₄, C₁-C₃, C₁-C₂, C₁), alkoxy (e.g., C₁-C₆, C₁-C₅, C₁-C₄, C₁-C₃, C₁-C₂, C₁), substituted alkoxy (e.g., C₁-C₆, C₁-C₅, C₁-C₄, C₁-C₃, C₁-C₂, C₁), haloalkyl (e.g., CF₃), alkenyl (e.g., C₂-C₆, C₂-C₅, C₂-C₄, C₂-C₃, C₂), substituted alkenyl (e.g., C₂-C₆, C₂-C₅, C₂-C₄, C₂-C₃, C₂), alkynyl (e.g., C₂-C₆, C₂-C₅, C₂-C₄, C₂-C₃, C₂), substituted alkynyl (e.g., C₂-C₆, C₂-C₅, C₂-C₄, C₂-C₃, C₂), cyclic (e.g., C₃-C₁₂, C₅-C₆), substituted cyclic (e.g., C₃-C₁₂, C₅-C₆), carbocyclic (e.g., C₃-C₁₂, C₅-C₆), substituted carbocyclic (e.g., C₃-C₁₂, C₅-C₆), heterocyclic (e.g., C₃-C₁₂, C₅-C₆), substituted heterocyclic (e.g., C₃-C₁₂, C₅-C₆), aryl (e.g., benzyl and phenyl), substituted aryl (e.g., substituted benzyl or phenyl), heteroaryl (e.g., pyridyl or pyrimidyl), substituted heteroaryl (e.g., substituted pyridyl or pyrimidyl), aralkyl (e.g., benzyl), substituted aralkyl (e.g., substituted benzyl), halo, hydroxyl, aryloxy (e.g., C₆-C₁₂, C₆), substituted aryloxy (e.g., C₆-C₁₂, C₆), alkylthio (e.g., C₁-C₆), substituted alkylthio (e.g., C₁-C₆), arylthio (e.g., C₆-C₁₂, C₆), substituted arylthio (e.g., C₆-C₁₂, C₆), cyano, carbonyl, substituted carbonyl, carboxyl, substituted carboxyl, amino, substituted amino, amido, substituted amido, thio, substituted thio, sulfinyl, substituted sulfinyl, sulfonyl, substituted sulfonyl, sulfinamide, substituted sulfinamide, sulfonamide, substituted sulfonamide, urea, substituted urea, carbamate, substituted carbamate, amino acid, and peptide groups.

As used herein, the phrase “optionally substituted with one or more halogen(s)” or “optionally substituted with C₆-C₁₀ aryl group(s)”, means at least one or more of said functional group provided that such substitution is in accordance with permitted valence of the substituted atom and the substituent.

As used herein, the term “analog” refers to a compound having a structure similar to that of another compound, but differing from it in respect to a certain component. It can differ in one or more atoms, functional groups, or substructures, which are replaced with other atoms, functional groups, or substructures.

The term “binding” as it relates to interaction between the targeting ligand and the targeted protein which is ALK or ALK and FAK, refers to an inter-molecular interaction that is substantially specific in that binding of the targeting ligand with other kinases and other proteinaceous entities present in the cell is functionally insignificant. Present bispecific compounds preferentially bind and recruit ALK or ALK and FAK for targeted degradation, including mutant forms thereof (e.g., EML4-ALK including the G1202R and L1196M mutants, and NPM-ALK) that manifest themselves in pathological states.

The term “binding” as it relates to interaction between the degron and the E3 ubiquitin ligase, typically refers to an inter-molecular interaction that may or may not exhibit an affinity level that equals or exceeds that affinity between the targeting ligand and the target protein, but nonetheless wherein the affinity is sufficient to achieve recruitment of the ligase to the targeted degradation and the selective degradation of the targeted protein.

Broadly, the bispecific compounds of the present invention have a structure represented by formula (I):

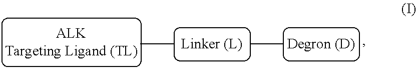

wherein the targeting ligand represents a moiety that binds ALK or ALK and FAK, the degron represents a moiety that binds an E3 ubiquitin ligase, and the linker represents a moiety that connects covalently the degron and the targeting ligand, or a pharmaceutically acceptable salt or stereoisomer thereof, wherein the ALK targeting ligand is a brigatinib analog, a ceritinib analog, or a [6-{[(1S)-1-(5-fluoropyridin-2-yl)ethyl]amino}-1-(5-methyl-1H-pyrazol-3-yl)-1H-pyrrolo[2,3-b]pyridine analog.

### Targeting Ligands

In some embodiments, the ALK targeting ligand is a brigatinib analog.

In some embodiments, the brigatinib analog has a structure represented by formula TL-1:

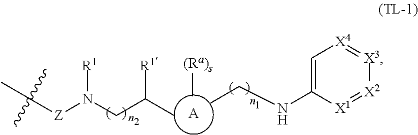

wherein
X₁ is N or CR^{b};
X² is N or CR^{c};
X³ is N or CR^{d};
X⁴ is N or CR^{e};
A is an aryl or a 5- or 6-membered heteroaryl ring which contains 1 to 4 heteroatoms selected from N, O and S(O)ᵣ;
r is 0, 1 or 2;
R^{a}, R^{b}, R^{c}, R^{d} and R^{e} are independently halo, —CN, —NO₂, —R¹, —OR², —O—NR¹R², —NR¹R², —NR¹—NR¹R², —NR¹—OR², —C(O)YR², —OC(O)YR², —NR¹C(O)YR², —SC(O)YR², —NR¹C(═S)YR², —OC(═S)YR², —C(═S)YR², —YC(═NR¹)YR², —YC(═N—OR¹)YR², —YC(═N—NR¹R²)YR², —YP(═O)(YR³)(YR³), —Si(R^{3a})₃, —NR¹SO₂R², —S(O)ᵣR², —SO₂NR¹R² or —NR¹SO₂NR¹R²; wherein each Y is independently a bond, —O—, —S— or —NR¹—; or
alternatively two adjacent substituents selected from R^{b}, R^{c}, R^{d}, and R^{e}; or two adjacent R^{a} moieties, with the atoms to which they are attached, form a 5-, 6- or 7-membered carbocyclic or heterocyclic ring, which contains 0-4 heteroatoms selected from N, O and S(O)ᵣ and which is substituted with one to four R^{f} moieties wherein,
each R^{f} moiety is independently halo, ═O, ═S, —CN, —NO₂, —R¹, —OR², —O—NR¹R², —NR¹R², —NR¹—NR¹R², —NR¹—OR², —C(O)YR², —OC(O)YR², —NR¹C(O)YR², —SC(O)YR², —NR¹C(═S)YR², —OC(═S)YR², —C(═S)YR², —YC(═NR¹)YR², —YC(═N—OR¹)YR², —YC(═N—NR¹R²)YR², —YP(═O)(YR³)(YR³), —Si(R^{3a})₃, —NR¹SO₂R², —S(O)ᵣR², —SO₂NR¹R² or —NR¹SO₂NR¹R²; or alternatively two adjacent R^{f} moieties with the atoms to which they are attached, form a 5-, 6- or 7-membered carbocyclic or heterocyclic ring, which contains 0-4 heteroatoms selected from N, O and S(O)ᵣ; provided that at least one of R^{a}, R^{b}, R^{c}, R^{d}, R^{e}, and R^{f}, when present, is or contains —P(═O)(R³)₂ or a ring system containing the moiety —P(═O)(R³)₂ as a ring member;
s is 1, 2, 3 or 4;
n₁ is 0 or 1;
n₂ is 1 or 2;
each R¹, R^{1′}, and R² is independently H, alkyl, alkenyl, alkynyl, cycloalkyl, cycloalkenyl, cycloalkynyl, aryl, heteroalkyl, heterocyclic or heteroaryl,
or R¹ and R^{1′} together with the atoms to which they are attached form a 5- to 6-membered heterocyclyl,
or R¹ and Z together with the atoms to which they are attached form a 4- to 7-membered heterocyclyl, which in some embodiments, is a bicyclic group, otherwise Z is absent;
each R³ is independently H, alkyl, alkenyl, alkynyl, cycloalkyl, cycloalkenyl, cycloalkynyl, aryl, heteroalkyl, heterocyclic or heteroaryl, or two adjacent R³ moieties combine to form a ring system including a phosphorous atom;
each R^{3a} is independently H, alkyl, alkenyl, alkynyl, cycloalkyl, cycloalkenyl, cycloalkynyl, aryl, heteroalkyl, heterocyclic or heteroaryl;
alternatively, each NR¹R² moiety independently is a 5-, 6- or 7-membered carbocyclic or heterocyclic ring, which can be optionally substituted and which contains 0-2 additional heteroatoms selected from N, O and S(O)ᵣ; and
each of the foregoing alkyl, alkenyl, alkynyl, cycloalkyl, cycloalkenyl, cycloalkynyl, aryl, heteroalkyl, heteroaryl and heterocyclic moieties is optionally substituted;
provided that when R^{a} is methoxy, s is not 1.

In some embodiments, the ring structures formed by R¹ and Z, together with the atoms to which they are attached, provide rigidity to the bispecific compounds, which results in less freedom of rotation with respect to the targeting ligand and degron.

Therefore, in some embodiments, the bispecific compounds of the present invention have a structure as represented by formula I-1:

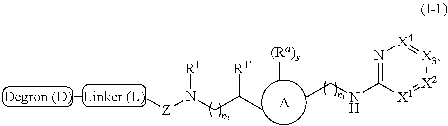

or a pharmaceutically acceptable salt or stereoisomer thereof.

In some embodiments, Z is absent and the ALK targeting ligand has a structure represented by formula TL-1a:

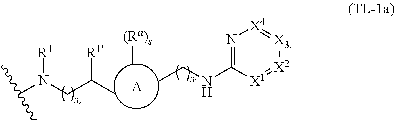

In some embodiments, n₁ is 0, n₂ is 1, R^{1′} is H, X² is CR^{c} wherein R^{c} is

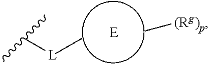

and the ALK targeting ligand has a structure represented by formula TL-1a1:

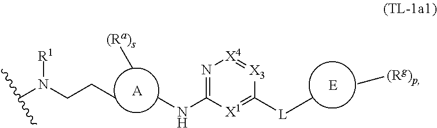

wherein
E is an aryl or a 5- or 6-membered heteroaryl ring which contains 1 to 4 heteroatoms selected from N, O and S(O)ᵣ;
r is 0, 1 or 2;
each R^{g} is independently halo, —CN, —NO₂, —R¹, —OR², —O—NR¹R², —NR¹R², —NR¹—NR¹R², —NR¹—OR², —C(O)YR², —OC(O)YR², —NRIC(O)YR², —SC(O)YR², —NRIC(═S)YR², —OC(═S)YR², —C(═S)YR², —YC(═NR¹)YR², —YC(═N—OR¹)YR², —YC(═N—NR¹R²)YR², —YP(═O)(YR³)(YR³), —Si(R^{3a})₃, —NR¹SO₂R², —S(O)ᵣR², —SO₂NR¹R² or —NR¹SO₂NR¹R²; or alternatively, each R^{g} may also be or include an independently selected moiety, —P(═O)(R³)₂ or a ring system containing the moiety —P(═O)(R³)₂ as a ring member;
at least one of R^{a} and R^{g} is or contains —P(═O)(R³)₂ or a ring system containing the moiety —P(═O)(R³)₂ as a ring member;

### L is O or NH; and

p is 1, 2, 3 or 4.

Therefore, in some embodiments, the bispecific compounds of the present invention have a structure as represented by formula I-1a1:

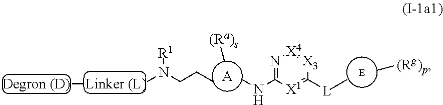

or a pharmaceutically acceptable salt or stereoisomer thereof.

In some embodiments, A and E are each phenyl and the ALK targeting ligand has a structure represented by formula TL-1a1a:

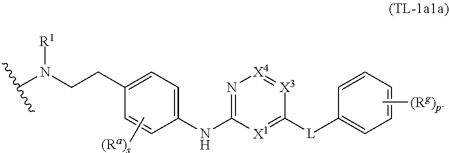

Therefore, in some embodiments, the bispecific compounds of the present invention have a structure represented by formula I-1a1a:

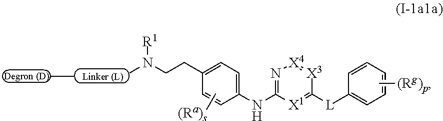

or a pharmaceutically acceptable salt or stereoisomer thereof.

In some embodiments, X¹ is N, X³ is C—Cl, X⁴ is CH, L is NH, R¹ is H, R^{a} is independently Me or OMe, s is 2, R^{g} is —P(═O)(Me)₂ and p is 1, and the ALK targeting ligand has a structure represented by formula TL-1a1a1:

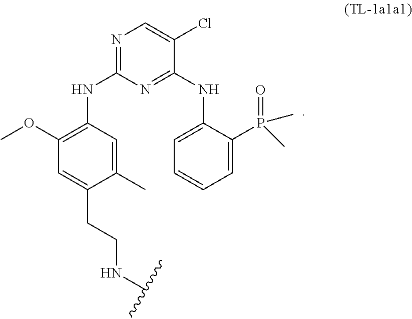

Therefore, in some embodiments, the bispecific compounds of the present invention have a structure represented by formula I-1a1a1:

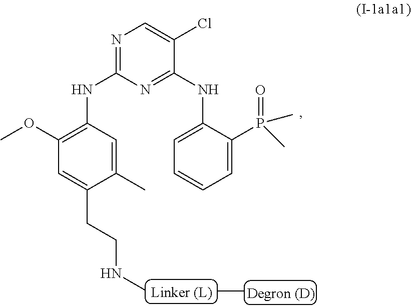

or a pharmaceutically acceptable salt or stereoisomer thereof.

In some embodiments, X¹ is N, X³ is C—Cl, X⁴ is CH, L is NH, R¹ is Me, R^{a} is independently Me or OMe, s is 2, R^{g} is —P(═O)(Me)₂ and p is 1, and the ALK targeting ligand has a structure represented by formula TL-1a1a2:

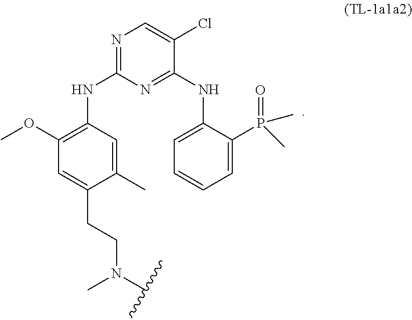

Therefore, in some embodiments, the bispecific compounds of the present invention have a structure represented by formula I-1a1a2:

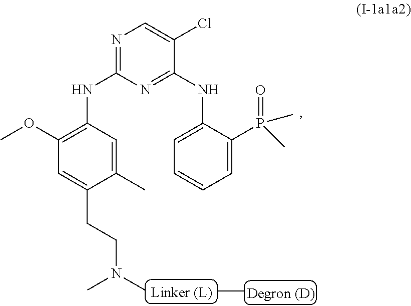

Linker (L) Degron (D) (I-1a1a2), or a pharmaceutically acceptable salt or stereoisomer thereof.

In some embodiments, n₁ is 0, n₂ is 2, R¹ and R^{1′} together with the atoms to which they are attached form a piperidinyl ring, X² is CR^{c} wherein R^{c} is

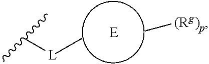

and the ALK targeting ligand has a structure represented by formula TL-1a2:

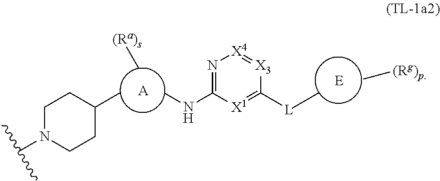

Therefore, in some embodiments, the bispecific compounds of the present invention have a structure represented by formula I-1a2:

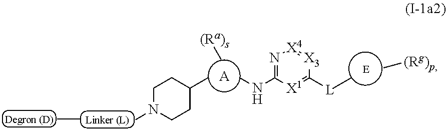

or a pharmaceutically acceptable salt or stereoisomer thereof.

In some embodiments, A and E are each phenyl and the ALK targeting ligand has a structure represented by formula TL-1a2a:

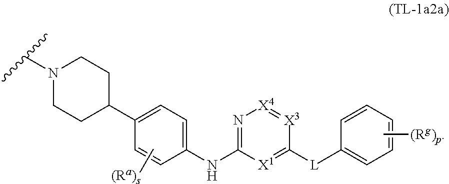

Therefore, in some embodiments, the bispecific compounds of the present invention have a structure as represented by formula I-1a2a:

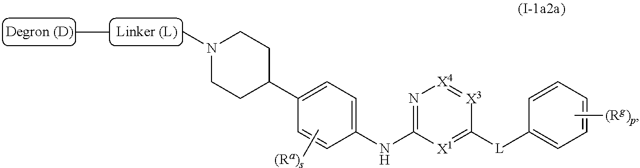

or a pharmaceutically acceptable salt or stereoisomer thereof.

In some embodiments, X¹ is N, X³ is C—Cl, X⁴ is CH, L is NH, R^{a} is independently Me or OMe, s is 2, R^{g} is —P(═O)(Me)₂ and p is 1, and the ALK targeting ligand has a structure represented by formula TL-1a2a1:

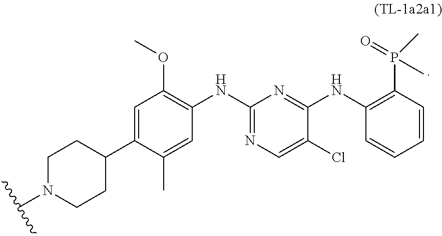 [SMILES: *N1CCC(c2cc(OC)c(Nc3ncc(Cl)c(Nc4ccccc4P(C)(C)=O)n3)cc2C)CC1]

Therefore, in some embodiments, the bispecific compounds of the present invention have a structure as represented by formula I-1a2a1:

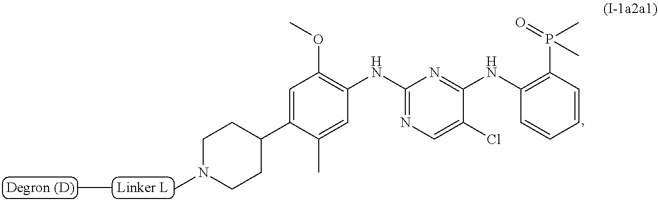

or a pharmaceutically acceptable salt or stereoisomer thereof.

In some embodiments, R¹ and Z together with the atoms to which they are attached form a 4- to 7-membered heterocyclyl, which in some embodiments is a bicyclic group, and the ALK targeting ligand has a structure represented by formula TL-1b:

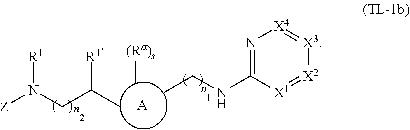

Therefore, in some embodiments, the bispecific compounds of the present invention have a structure represented by formula I-1b:

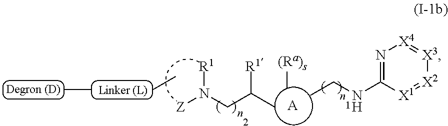

or a pharmaceutically acceptable salt or stereoisomer thereof.

In some embodiments, R¹ and Z, together with the atoms to which they are attached, form a 2,6-diazospiro[3.3]heptanyl, piperidinyl, or piperazinyl group.

In some embodiments, the brigatinib analog has a structure represented by formula TL-1′:

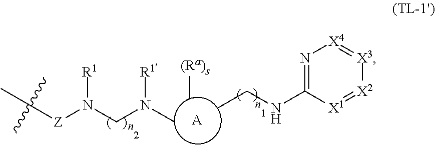

wherein
X¹ is N or CR^{b};
X² is N or CR^{c};
X³ is N or CR^{d};
X⁴ is N or CR^{e};
A is an aryl or a 5- or 6-membered heteroaryl ring which contains 1 to 4 heteroatoms selected from N, O and S(O)ᵣ;
r is 0, 1 or 2;
R^{a}, R^{b}, R^{c}, R^{d} and R^{e} are independently halo, —CN, —NO₂, —R¹, —OR², —O—NR¹R², —NR¹R², —NR¹—NR¹R², —NR¹—OR², —C(O)YR², —OC(O)YR², —NR¹C(O)YR², —SC(O)YR², —NR¹C(═S)YR², —OC(═S)YR², —C(═S)YR², —YC(═NR¹)YR², —YC(═N—OR¹)YR², —YC(═N—NR¹R²)YR², —YP(═O)(YR³)(YR³), —Si(R^{3a})₃, —NR¹SO₂R², —S(O)ᵣR², —SO₂NR¹R² or —NR¹SO₂NR¹R²; wherein each Y is independently a bond, —O—, —S— or —NR¹—; or
alternatively two adjacent substituents selected from R^{b}, R^{c}, R^{d}, and R^{e}; or two adjacent R^{a} moieties, with the atoms to which they are attached, form a 5-, 6- or 7-membered carbocyclic or heterocyclic ring, which contains 0-4 heteroatoms selected from N, O and S(O)ᵣ and which is substituted with one to four R^{f} moieties wherein,
each R^{f} moiety is independently halo, ═O, ═S, —CN, —NO₂, —R¹, —OR², —O—NR¹R², —NR¹R², —NR¹—NR¹R², —NR¹—OR², —C(O)YR², —OC(O)YR², —NR¹C(O)YR², —SC(O)YR², —NR¹C(═S)YR², —OC(═S)YR², —C(═S)YR², —YC(═NR¹)YR², —YC(═N—OR¹)YR², —YC(═N—NR¹R²)YR², —YP(═O)(YR³)(YR³), —Si(R^{3a})₃, —NR¹SO₂R², —S(O)ᵣR², —SO₂NR¹R² or —NR¹SO₂NR¹R²; or alternatively two adjacent R^{f} moieties with the atoms to which they are attached, form a 5-, 6- or 7-membered carbocyclic or heterocyclic ring, which contains 0-4 heteroatoms selected from N, O and S(O)ᵣ; provided that at least one of R^{a}, R^{b}, R^{c}, R^{d}, R^{e}, and R^{f}, when present, is or contains —P(═O)(R³)₂ or a ring system containing the moiety —P(═O)(R³)₂ as a ring member;
s is 1, 2, 3 or 4;
n₁ is 0 or 1;
n₂ is 1 or 2;
each R¹, R^{1′}, and R² is independently H, alkyl, alkenyl, alkynyl, cycloalkyl, cycloalkenyl, cycloalkynyl, aryl, heteroalkyl, heterocyclic or heteroaryl,
or R¹ and R^{1′} together with the atoms to which they are attached form a 5- to 6-membered heterocyclyl,
or R¹ and Z together with the atoms to which they are attached form a 4- to 7-membered heterocyclyl, which in some embodiments, is a bicyclic group, otherwise Z is absent;
each R³ is independently H, alkyl, alkenyl, alkynyl, cycloalkyl, cycloalkenyl, cycloalkynyl, aryl, heteroalkyl, heterocyclic or heteroaryl, or two adjacent R³ moieties combine to form a ring system including a phosphorous atom;
each R^{3a} is independently H, alkyl, alkenyl, alkynyl, cycloalkyl, cycloalkenyl, cycloalkynyl, aryl, heteroalkyl, heterocyclic or heteroaryl;
alternatively, each NR¹R² moiety independently is a 5-, 6- or 7-membered carbocyclic or heterocyclic ring, which can be optionally substituted and which contains 0-2 additional heteroatoms selected from N, O and S(O)ᵣ; and
each of the foregoing alkyl, alkenyl, alkynyl, cycloalkyl, cycloalkenyl, cycloalkynyl, aryl, heteroalkyl, heteroaryl and heterocyclic moieties is optionally substituted.

Therefore, in some embodiments, the bispecific compounds of the present invention have a structure represented by formula I-1′:

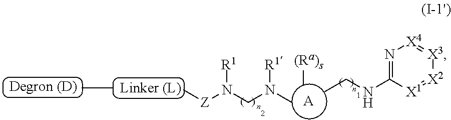

or a pharmaceutically acceptable salt or stereoisomer thereof.

In some embodiments, Z is absent and the ALK targeting ligand has a structure represented by formula TL-1′a:

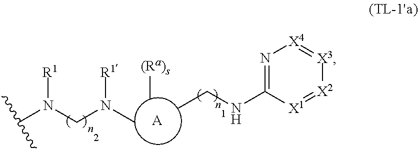

In some embodiments, n₁ is 0, n₂ is 2, R¹ and R^{1′} together with the atoms to which they are attached form piperazinyl, X² is CR^{c} wherein R^{c} is

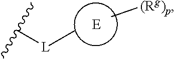

and the ALK targeting ligand has a structure represented by formula TL-1′a1:

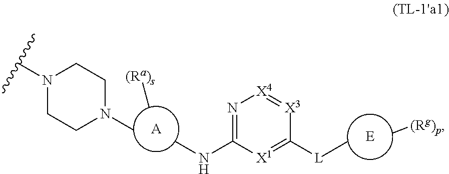

wherein
E is an aryl or a 5- or 6-membered heteroaryl ring which contains 1 to 4 heteroatoms selected from N, O and S(O)ᵣ;
r is 0, 1 or 2;
each R^{g} is independently halo, —CN, —NO₂, —R¹, —OR², —O—NR¹R², —NR¹R², —NR¹—NR¹R², —NR¹—OR², —C(O)YR², —OC(O)YR², —NRIC(O)YR², —SC(O)YR², —NRIC(═S)YR², —OC(═S)YR², —C(═S)YR², —YC(═NR¹)YR², —YC(═N—OR¹)YR², —YC(═N—NR¹R²)YR², —YP(═O)(YR³)(YR³), —Si(R^{3a})₃, —NR¹SO₂R², —S(O)ᵣR², —SO₂NR¹R² or —NR¹SO₂NR¹R²; or alternatively, each R^{g} may also be or include an independently selected moiety, —P(═O)(R³)₂ or a ring system containing the moiety —P(═O)(R³)₂ as a ring member;
at least one of R^{a} and R^{g} is or contains —P(═O)(R³)₂ or a ring system containing the moiety —P(═O)(R³)₂ as a ring member;

### L is O or NH; and

p is 1, 2, 3 or 4.

Therefore, in some embodiments, the bispecific compounds of the present invention have a structure as represented by formula I-1′a1:

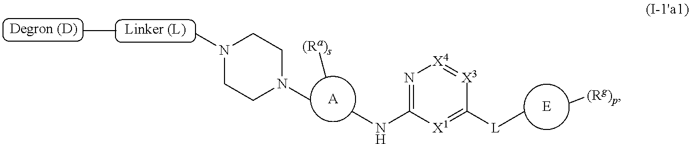

or a pharmaceutically acceptable salt or stereoisomer thereof.

In some embodiments, A and E are each phenyl and the ALK targeting ligand has a structure represented by formula TL-1′a1a:

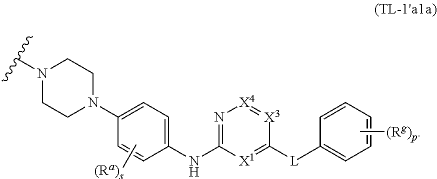

Therefore, in some embodiments, the bispecific compounds of the present invention have a structure represented by formula I-1′a1a:

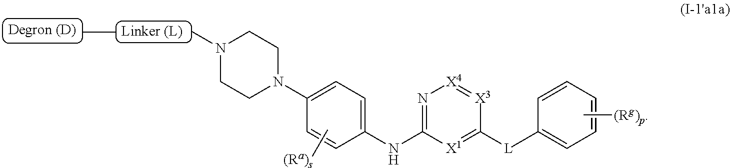

or a pharmaceutically acceptable salt or stereoisomer thereof.

In some embodiments, X¹ is N, X³ is C—Cl, X⁴ is CH, L is NH, R^{a} is OMe, s is 1, R^{g} is —P(═O)(Me)₂ and p is 1, and the ALK targeting ligand has a structure represented by formula TL-1′a1a1:

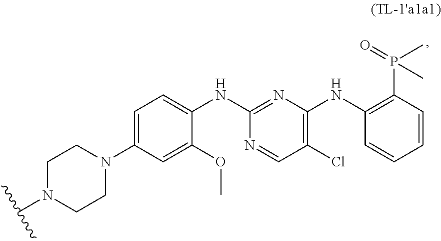 [SMILES: *N1CCN(c2ccc(Nc3ncc(Cl)c(Nc4ccccc4P(C)(C)=O)n3)c(OC)c2)CC1]

Therefore, in some embodiments, the bispecific compounds of the present invention have a structure as represented by formula I-1′a1a1:

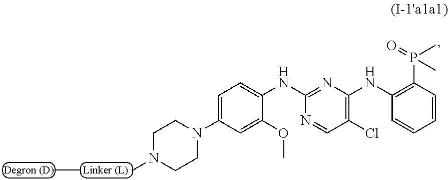

or a pharmaceutically acceptable salt or stereoisomer thereof.

Yet other brigatinib analogs that may be suitable for use in the bispecific compounds of the present invention are described in U.S. Pat. No. 9,012,462.

In some embodiments, the targeting ligand is a ceritinib analog.

In some embodiments, the ceritinib analog has a structure represented by formula TL-2:

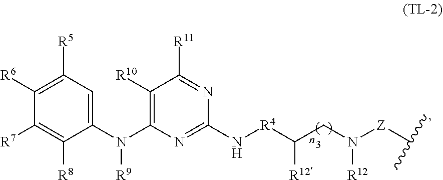

wherein
R⁴ is C₆₋₁₀ aryl, C₅₋₁₀ heteroaryl, C₃₋₁₂ cycloalkyl or C₃₋₁₀ heterocycloalkyl, wherein R⁴ is optionally substituted by R¹³, R¹⁴, R¹⁵, or R¹⁶; or wherein two adjacent substituents on R⁴ may form, together with the carbon atoms to which they are attached, an unsubstituted or substituted 5- or 6-membered carbocyclic or heterocyclic ring containing 0, 1, 2 or 3 heteroatoms selected from N, O and S;
R⁵, R⁶, R⁷, and R⁸ are independently hydrogen, C₁-C₈ alkyl, C₂-C₈ alkenyl, C₂-C₈ alkynyl, C₃-C₈ cycloalkyl, C₃-C₈ cycloalkyl-C₁-C₈ alkyl, C₅-C₁₀ aryl-C₁-C₈ alkyl, hydroxyl-C₁-C₈ alkyl, C₁-C₈ alkoxy-C₁-C₈ alkyl, amino-C₁-C₈ alkyl, halo-C₁-C₈ alkyl, unsubstituted or substituted C₅-C₁₀ aryl, unsubstituted or substituted 5 or 6 membered heterocyclyl containing 1, 2 or 3 heteroatoms selected from N, O, and S, hydroxy, C₁-C₈ alkoxy, hydroxyl-C₁-C₈ alkoxy, C₁-C₈ alkoxy-C₁-C₈ alkoxy, halo-C₁-C₈ alkoxy, unsubstituted or substituted C₅-C₁₀ aryl-C₁-C₈ alkoxy, unsubstituted or substituted heterocyclyloxy, unsubstituted or substituted heterocyclyl-C₁-C₈ alkoxy, unsubstituted or substituted amino, C₁-C₈ alkylthio, C₁-C₈ alkylsulfinyl, C₁-C₈ alkylsulfonyl, C₅-C₁₀ arylsulfonyl, halogen, carboxy, C₁-C₈ alkoxycarbonyl, unsubstituted or substituted carbamoyl, unsubstituted or substituted sulfamoyl, cyano, nitro, —S(O)₀₋₂NR₁₉R₂₀, —S(O)₀₋₂R₂₀, —NR₁₉S(O)₀₋₂R²⁰, —C(O)NR₁₉R₂₀, —C(O)R₂₀, or —C(O)OR₂₀; wherein R₁₉ is hydrogen or C₁₋₆ alkyl; and R₂₀ is hydrogen, C₁₋₆ alkyl or C₃₋₁₂ cycloalkyl;
or R⁵ and R⁶, R⁶ and R⁷, and/or R⁷ and R⁸, together with the carbon atoms to which they are attached, form a 5- or 6-membered carbocyclic or heterocyclic ring containing 0, 1, 2 or 3 heteroatoms selected from N, O and S;
R⁹ is hydrogen or C₁₋₈alkyl;
each of R¹⁰ and R¹¹ independently is hydrogen, C₁₋₈ alkyl, C₁₋₈ alkoxy-C₁₋₈ alkyl, halo-C₁₋₈ alkyl, C₁₋₈ alkoxy, halogen, carboxy, C₁₋₈ alkoxycarbonyl, unsubstituted or substituted carbamoyl, cyano, or nitro;
R¹² and R^{12′} are independently hydrogen or C₁₋₆ alkyl,
or R¹² and R^{12′}, together with the atoms to which they are attached form a 5- to 6-membered heterocyclyl,
or R¹² and Z, together with the atoms to which they are attached, form a 4- to 7-membered heterocyclyl, which in some embodiments is a bicyclic group; otherwise Z is absent;
R¹³, R¹⁴, R¹⁵, and R¹⁶ are independently hydrogen, C₁-C₈ alkyl, C₂-C₈ alkenyl, C₂-C₈ alkynyl, C₃-C₈ cycloalkyl, C₃-C₈ cycloalkyl-C₁-C₈ alkyl, C₅-C₁₀ aryl-C₁-C₈ alkyl, hydroxyl-C₁-C₈ alkyl, C₁-C₈ alkoxy-C₁-C₈ alkyl, amino-C₁-C₈ alkyl, halo-C₁-C₈ alkyl, unsubstituted or substituted C₅-C₁₀ aryl, unsubstituted or substituted 5 or 6 membered heterocyclyl containing 1, 2 or 3 heteroatoms selected from N, O, and S, hydroxy, C₁-C₂ alkoxy, hydroxyl-C₁-C₈ alkoxy, C₁-C₈ alkoxy-C₁-C₈ alkoxy, halo-C₁-C₈ alkoxy, unsubstituted or substituted C₅-C₁₀ aryl-C₁-C₈ alkoxy, unsubstituted or substituted heterocyclyloxy, unsubstituted or substituted heterocyclyl-C₁-C₈ alkoxy, unsubstituted or substituted amino, C₁-C₈ alkylthio, C₁-C₈ alkylsulfinyl, C₁-C₈ alkylsulfonyl, C₅-C₁₀ arylsulfonyl, halogen, carboxy, C₁-C₈ alkoxycarbonyl, unsubstituted or substituted carbamoyl, unsubstituted or substituted sulfamoyl, cyano, nitro, —S(O)₀₋₂NR₁₉R₂₀, —S(O)₀₋₂R₁₉, —C(O)R₁₈, —CXR₁₈, —NR₁₉XR₁₈, —NR₁₉XNR₁₉R₂₀, —OXNR₁₉R₂₀, —OXOR₁₉, or —XR₁₈;
X is a bond or C₁₋₆ alkylene;
R¹⁸ is C₆₋₁₀ aryl, C₅₋₁₀ heteroaryl, C₃₋₁₂ cycloalkyl or C₃₋₁₀ heterocycloalkyl;
R₁₉ and R₂₀ are independently hydrogen or C₁₋₆ alkyl;
n₃ is 1 or 2;
and any aryl, heteroaryl, cycloalkyl, or heterocycloalkyl of R¹⁸ is optionally substituted by 1 to 3 radicals independently selected from C₁₋₆ alkyl, C₃₋₁₀ heterocycloalkyl-C₀₋₄ alkyl optionally substituted with C₁₋₆ alkyl, —C(O)R₁₉, —C(O)N₁₉R₂₀, —XNR₁₉R₂₀, —NR₁₉XNR₁₉R₂₀, and —NR₁₉C(O)R₂₀; wherein X is a bond or C₁₋₆alkylene.

In some embodiments, the ring structures formed by R¹² and Z, together with the atoms to which they are attached, provide rigidity to the bispecific compounds, which results in less freedom of rotation with respect to the targeting ligand and degron.

In some embodiments, the ring structures formed by R¹ and Z, together with the atoms to which they are attached, provide rigidity to the bispecific compounds which results in less freedom of rotation with respect to the targeting ligand and degrons.

Therefore, in some embodiments, the bispecific compounds of the present invention have a structure represented by formula I-2:

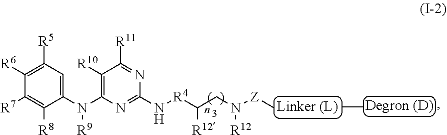

or a pharmaceutically acceptable salt or stereoisomer thereof.

In some embodiments, Z is absent and the ALK targeting ligand is a ceritinib analog that has a structure represented by formula TL-2a:

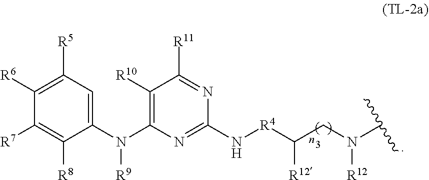

Therefore, in some embodiments, the bispecific compounds of the present invention have a structure represented by formula I-2:

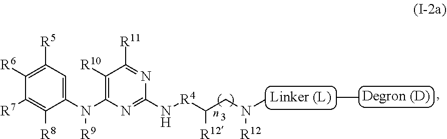

or a pharmaceutically acceptable salt or stereoisomer thereof.

In some embodiments, R⁴ is aryl optionally substituted with R¹⁷ and the ALK targeting ligand is a ceritinib analog that has a structure represented by formula TL-2a1:

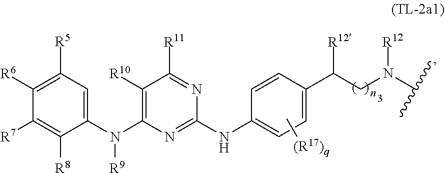

wherein
R¹⁷ is independently hydrogen, C₁-C₈ alkyl, C₂-C₈ alkenyl, C₂-C₈ alkynyl, C₃-C₈ cycloalkyl, C₃-C₈ cycloalkyl-C₁-C₈ alkyl, C₅-C₁₀ aryl-C₁-C₈ alkyl, hydroxyl-C₁-C₈ alkyl, C₁-C₈ alkoxy-C₁-C₈ alkyl, amino-C₁-C₈ alkyl, halo-C₁-C₈ alkyl, unsubstituted or substituted C₅-C₁₀ aryl, unsubstituted or substituted 5 or 6 membered heterocyclyl containing 1, 2 or 3 heteroatoms selected from N, O, and S, hydroxy, C₁-C₂ alkoxy, hydroxyl-C₁-C₈ alkoxy, C₁-C₈ alkoxy-C₁-C₈ alkoxy, halo-C₁-C₈ alkoxy, unsubstituted or substituted C₅-C₁₀ aryl-C₁-C₈ alkoxy, unsubstituted or substituted heterocyclyloxy, unsubstituted or substituted heterocyclyl-C₁-C₈ alkoxy, unsubstituted or substituted amino, C₁-C₈ alkylthio, C₁-C₈ alkylsulfinyl, C₁-C₈ alkylsulfonyl, C₅-C₁₀ arylsulfonyl, halogen, carboxy, C₁-C₈ alkoxycarbonyl, unsubstituted or substituted carbamoyl, unsubstituted or substituted sulfamoyl, cyano, nitro, —S(O)₀₋₂NR₁₉R₂₀, —S(O)₀₋₂R₁₉, —C(O)R₁₈, —CXR₁₈, —NR₁₉XR₁₈, —NR₁₉XNR₁₉R₂₀, —OXNR₁₉R₂₀, —OXOR₁₉, or —XR₁₈; and
q is 0, 1 or 2.

Therefore, in some embodiments, the bispecific compounds of the present invention have a structure as represented by formula I-2a1:

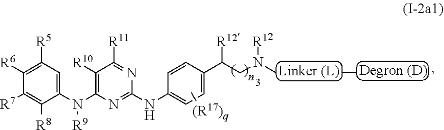

or a pharmaceutically acceptable salt or stereoisomer thereof.

In some embodiments, n₃ is 1, R⁵ is H, R⁶ is H, R⁷ is H, R⁸ is SO₂iPr, R⁹ is H, R¹⁰ is Cl, R¹¹ is H, R¹² is H, R^{12′} is H, R¹⁷ is independently Me or OMe, and q is 2, and the ALK targeting ligand has a structure represented by formula TL-2a1a:

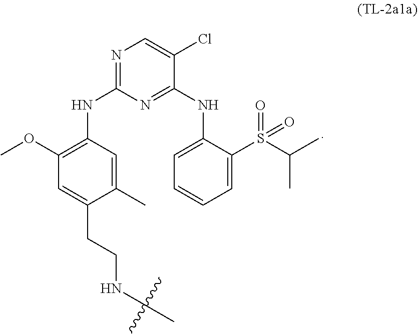 [SMILES: *NCCc1cc(OC)c(Nc2ncc(Cl)c(Nc3ccccc3S(=O)(=O)C(C)C)n2)cc1C]

Therefore, in some embodiments, the bispecific compounds of the present invention have a structure as represented by formula I-2a1a:

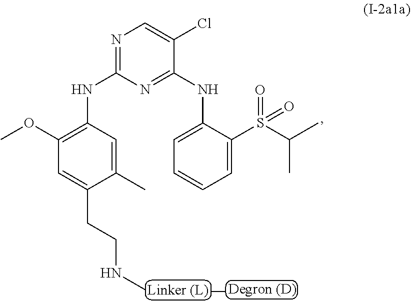

or a pharmaceutically acceptable salt or stereoisomer thereof.

In some embodiments, n₃ is 1, R⁵ is H, R⁶ is H, R⁷ is H, R⁸ is SO₂iPr, R⁹ is H, R¹⁰ is Cl, R¹¹ is H, R¹² is Me, R^{12′} is H, R¹⁷ is independently Me or OMe, and q is 2, and the ALK targeting ligand has a structure represented by formula TL-2a1b:

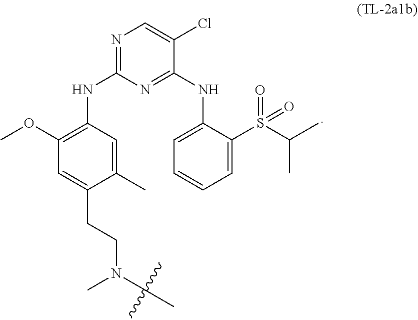 [SMILES: *N(C)CCc1cc(OC)c(Nc2ncc(Cl)c(Nc3ccccc3S(=O)(=O)C(C)C)n2)cc1C]

Therefore, in some embodiments, the bispecific compounds of the present invention have a structure as represented by formula I-2a1b:

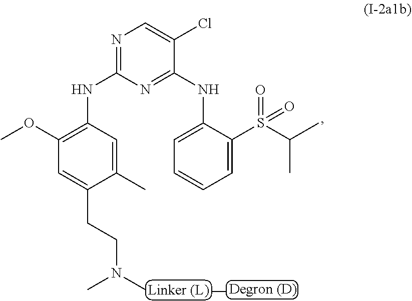

or a pharmaceutically acceptable salt or stereoisomer thereof.

In some embodiments, n₃ is 2, R⁵ is H, R⁶ is H, R⁷ is H, R⁸ is SO₂iPr, R⁹ is H, R¹⁰ is Cl, R¹¹ is H, R¹² and R^{12′} together with the atoms to which they are attached form a piperidinyl ring, R¹⁷ is independently Me or OMe, and q is 2, and the ALK targeting ligand has a structure represented by formula TL-2a1c:

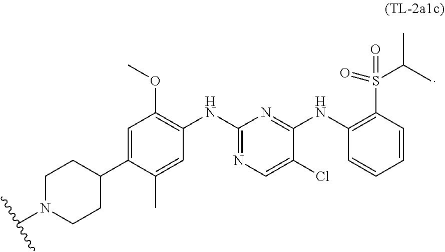 [SMILES: *N1CCC(c2cc(OC)c(Nc3ncc(Cl)c(Nc4ccccc4S(=O)(=O)C(C)C)n3)cc2C)CC1]

Therefore, in some embodiments, the bispecific compounds of the present invention have a structure represented by formula I-2a1c:

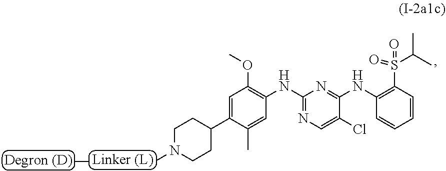

or a pharmaceutically acceptable salt or stereoisomer thereof.

In some embodiments, n₃ is 1, R⁵ is H, R⁶ is H, R⁷ is H, R⁸ is C(O)NHMe, R⁹ is H, R¹⁰ is Cl, R¹¹ is H, R¹² is Me, R^{12′} is H, R¹⁷ is independently Me or OMe, and q is 2, and the ALK targeting ligand has a structure represented by formula TL-2a1d:

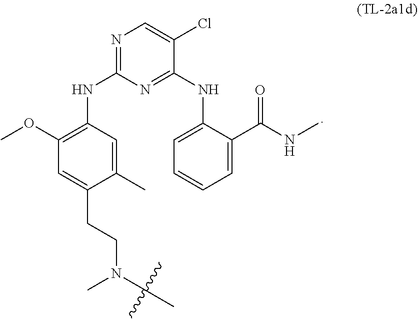

Therefore, in some embodiments, the bispecific compounds of the present invention have a structure as represented by formula I-2a1d:

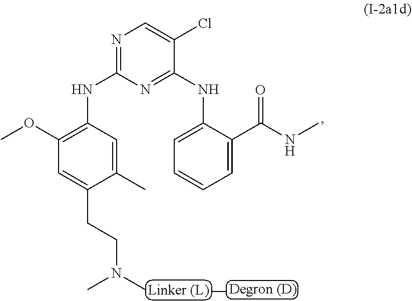

or a pharmaceutically acceptable salt or stereoisomer thereof.

In some embodiments, n₃ is 2, R⁵ is H, R⁶ is H, R⁷ is H, R⁸ is SO₂iPr, R⁹ is H, R¹⁰ is Cl, R₁₁ is H, R¹² and R^{12′} together with the atoms to which they are attached form a piperidinyl ring, R¹⁷ is independently Me or OEt, and q is 2, and the ALK targeting ligand has a structure represented by formula TL-2a1e:

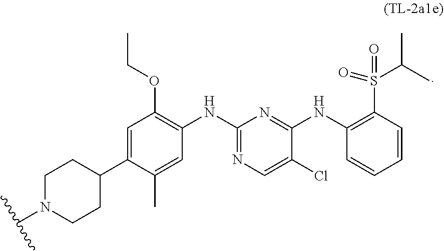 [SMILES: *N1CCC(c2cc(OCC)c(Nc3ncc(Cl)c(Nc4ccccc4S(=O)(=O)C(C)C)n3)cc2C)CC1]

Therefore, in some embodiments, the bispecific compounds of the present invention have a structure represented by formula I-2a1e:

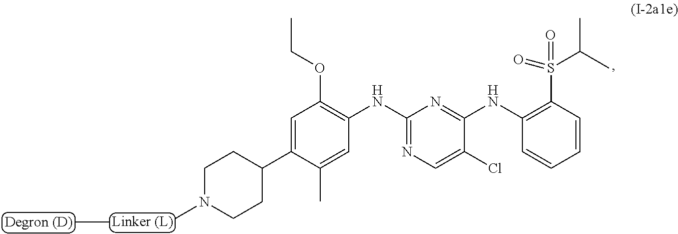

or a pharmaceutically acceptable salt or stereoisomer thereof.

In some embodiments, R¹² and Z, together with the atoms to which they are attached, form a 4- to 7-membered heterocyclyl and the ALK targeting ligand has a structure represented by formula TL-2b:

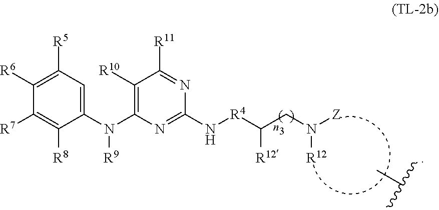

In some embodiments, R¹² and Z, together with the atoms to which they are attached, form 2,6-diazospiro[3.3]heptane, piperidine, or piperazine. In some embodiments, the TL binds to the linker via a nitrogen atom.

In some embodiments, R⁴ is aryl optionally substituted with R¹⁷ and the ALK targeting ligand is a ceritinib analog that has a structure represented by formula TL-2b1:

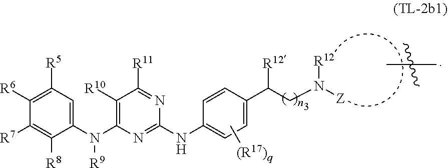

In some embodiments, n₃ is 1, R⁵ is H, R⁶ is H, R⁷ is H, R⁸ is SO₂iPr, R⁹ is H, R¹⁰ is Cl, R¹¹ is H, R^{12′} is H, R¹² and Z, together with the atoms to which they are attached, form 2,6-diazospiro[3.3]heptane, R¹⁷ is independently Me or OMe, and q is 2, and the ALK targeting ligand has a structure represented by formula TL-2b1a:

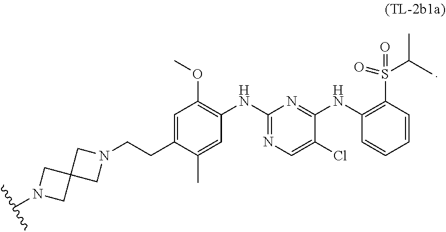 [SMILES: *N1CC2(C1)CN(CCc1cc(OC)c(Nc3ncc(Cl)c(Nc4ccccc4S(=O)(=O)C(C)C)n3)cc1C)C2]

Therefore, in some embodiments, the bispecific compounds of the present invention have a structure represented by formula I-2b1a:

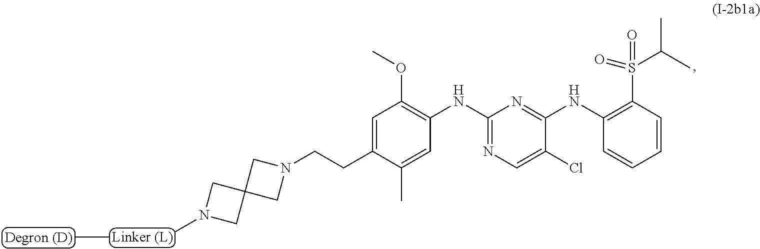

or a pharmaceutically acceptable salt or stereoisomer thereof.

In some embodiments, n₃ is 1, R⁵ is H, R⁶ is H, R⁷ is H, R⁸ is SO₂iPr, R⁹ is H, R¹⁰ is Cl, R¹¹ is H, R^{12′} is H, R¹² and Z together with the atoms to which they are attached form piperidinyl, R¹⁷ is independently Me or OMe, and q is 2, and the ALK targeting ligand has a structure represented by formula TL-2b1b:

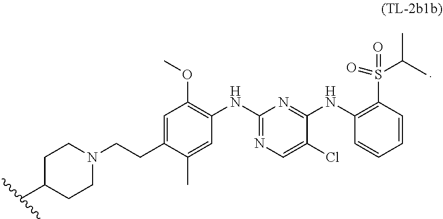 [SMILES: *C1CCN(CCc2cc(OC)c(Nc3ncc(Cl)c(Nc4ccccc4S(=O)(=O)C(C)C)n3)cc2C)CC1]

Therefore, in some embodiments, the bispecific compounds of the present invention have a structure as represented by formula I-2b1b:

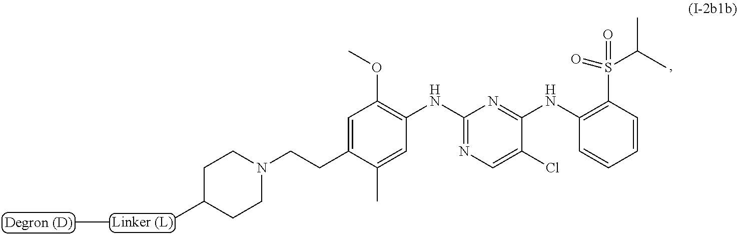

or a pharmaceutically acceptable salt or stereoisomer thereof.

In some embodiments, n₃ is 1, R⁵ is H, R⁶ is H, R⁷ is H, R⁸ is SO₂iPr, R⁹ is H, R¹⁰ is Cl, R¹¹ is H, R^{12′} is H, R¹² and Z together with the atoms to which they are attached form piperazinyl, R¹⁷ is independently Me or OMe, and q is 2, and the ALK targeting ligand has a structure represented by formula TL-2b1c:

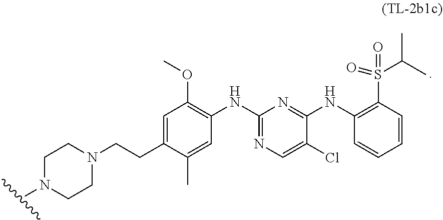

Therefore, in some embodiments, the bispecific compounds of the present invention have a structure as represented by formula I-2b1c:

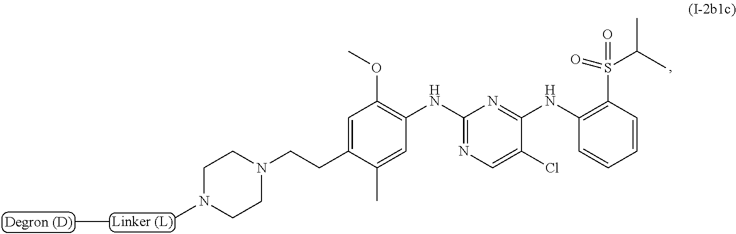

or a pharmaceutically acceptable salt or stereoisomer thereof.

Yet other ceritinib analogs that may be suitable for use in the compounds of the present invention are described in U.S. Pat. No. 7,893,074.

In some embodiments, the targeting ligand is a [6-{[(1S)-1-(5-fluoropyridin-2-yl)ethyl]amino}-1-(5-methyl-1H-pyrazol-3-yl)-1H-pyrrolo[2,3-b]pyridine analog.

In some embodiments, the [6-{[(1S)-1-(5-fluoropyridin-2-yl)ethyl]amino}-1-(5-methyl-1H-pyrazol-3-yl)-1H-pyrrolo[2,3-b]pyridine analog has a structure represented by formula TL-3:

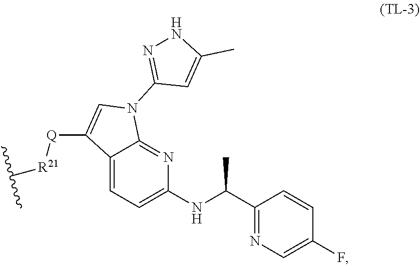

wherein
R²¹ is C₁-C₈ alkyl, C₂-C₈ alkenyl, C₂-C₈ alkynyl, C₃-C₈ cycloalkyl, C₃-C₈ cycloalkyl-C₁-C₈ alkyl, C₅-C₁₀ aryl-C₁-C₈ alkyl, hydroxyl-C₁-C₈ alkyl, C₁-C₈ alkoxy-C₁-C₈ alkyl, halo-C₁-C₈ alkyl, unsubstituted or substituted amino (e.g., amino-C₁-C₈ alkyl), unsubstituted or substituted C₅-C₁₀ aryl, or unsubstituted or substituted 5- or 6-membered heterocyclyl containing 1, 2 or 3 heteroatoms selected from N, O, and S; and

### Q is CH₂ or C(O).

Therefore, in some embodiments, the bispecific compounds of the present invention have a structure represented by formula I-3:

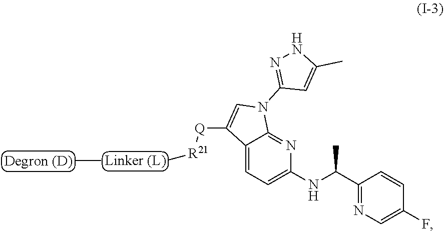

or a pharmaceutically acceptable salt or stereoisomer thereof.

In some embodiments, Q is C(O) and R²¹ is

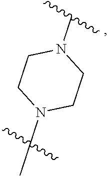 [SMILES: *N1CCN(*)CC1]

and the [6-{[(1S)-1-(5-fluoropyridin-2-yl)ethyl]amino}-1-(5-methyl-1H-pyrazol-3-yl)-1H-pyrrolo[2,3-b]pyridine analog has a structure represented by formula TL-3a:

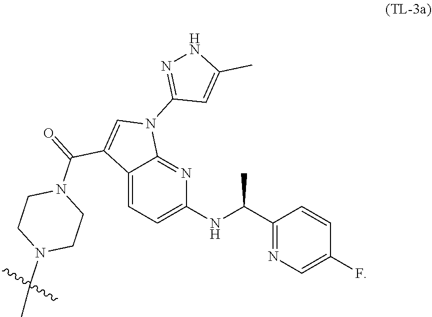

Therefore, in some embodiments, the bispecific compounds of the present invention have a structure represented by formula I-3a:

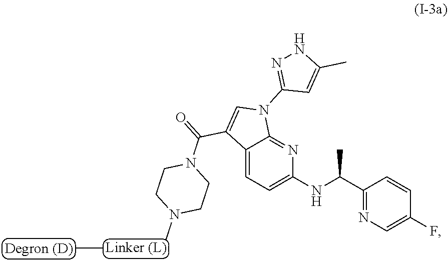

or a pharmaceutically acceptable salt or stereoisomer thereof.

In some embodiments, Q is C(O) and R²¹ is NMe, and the [6-{[(1S)-1-(5-fluoropyridin-2-yl)ethyl]amino}-1-(5-methyl-1H-pyrazol-3-yl)-1H-pyrrolo[2,3-b]pyridine analog has a structure represented by formula TL-3b:

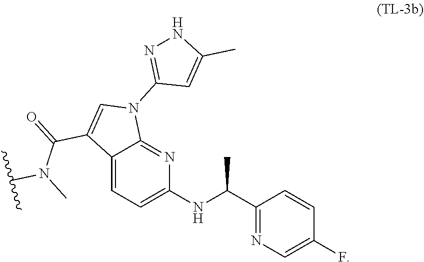 [SMILES: *N(C)C(=O)c1cn(-c2cc(C)[nH]n2)c2nc(N[C@@H](C)c3ccc(F)cn3)ccc12]

Therefore, in some embodiments, the bispecific compounds of the present invention have a structure as represented by formula I-3b:

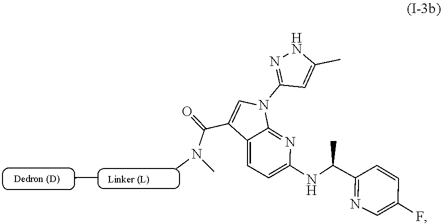

or a pharmaceutically acceptable salt or stereoisomer thereof.

### Linkers

The linker (“L”) provides a covalent attachment between the targeting ligand and the degron. The structure of linker may not be critical, provided it is substantially non-interfering with the activity of the targeting ligand or the degron.

In some embodiments, the linker may include an alkylene chain or a bivalent alkylene chain, either of which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR¹)—, —N(R′)C(NR¹)—, —C(NR¹)N(R′)—, —N(R′)C(NR¹)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃-C₁₂ carbocyclene, 3- to 12-membered heterocyclene, 5- to 12-membered heteroarylene or any combination thereof, wherein R′ is H or C₁-C₆ alkyl, wherein the interrupting and the one or both terminating groups may be the same or different.

In some embodiments, the linker includes an alkylene chain having 2-20 alkylene units. In some embodiments, the linker includes an alkylene chain having 3-12 alkylene units. In some embodiments, the linker may include a C₃-C₁₂ alkylene chain terminating in NH-group wherein the nitrogen is also bound to the degron.

“Carbocyclene” refers to a bivalent carbocycle radical, which is optionally substituted.

“Heterocyclene” refers to a bivalent heterocyclyl radical which may be optionally substituted.

“Heteroarylene” refers to a bivalent heteroaryl radical which may be optionally substituted.

Representative examples of linkers that may be suitable for use in the present invention include alkylene chains:

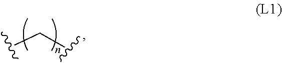

wherein n is an integer of 1-12 (“of” meaning inclusive), e.g., 1-12, 1-11, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10 and 1, 2, 3, 4, 5, 6, 7, 8, 9 and 10, examples of which include:

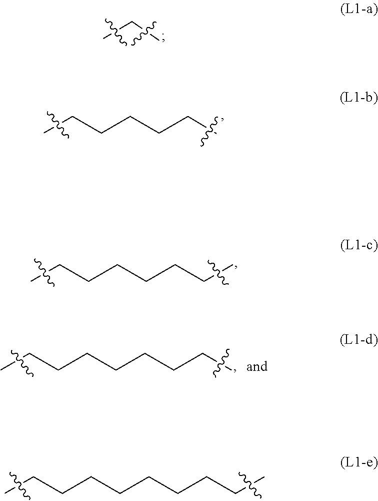

alkylene chains terminating in various functional groups (as described above), examples of which are as follows:

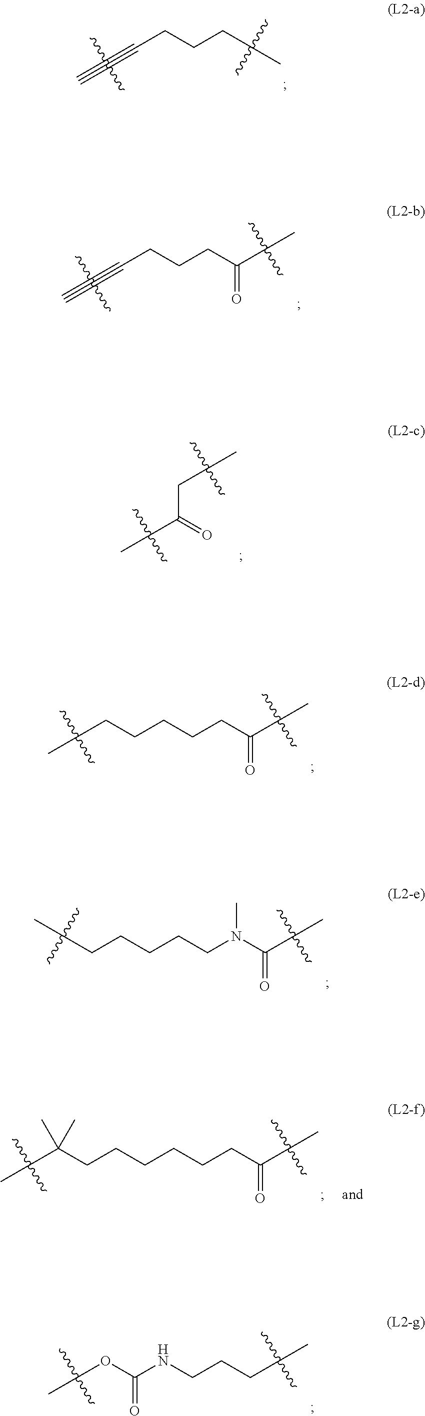

alkylene chains interrupted by various functional groups (as described above), examples of which are as follows:

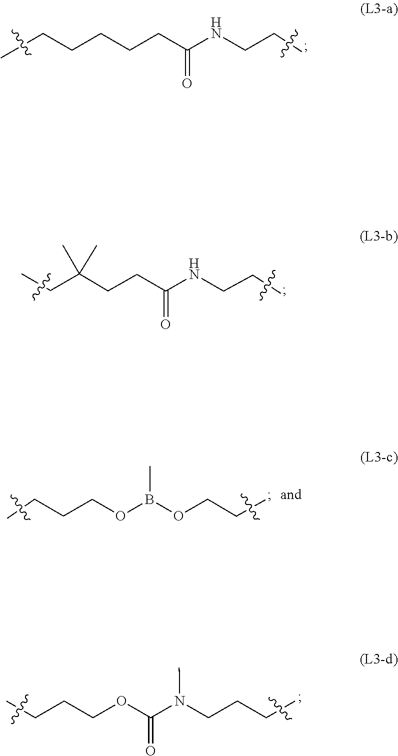

alkylene chains interrupted by or terminating with heterocyclene groups, e.g.,

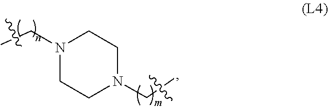

wherein m and n are independently integers of 0-10, examples of which include:

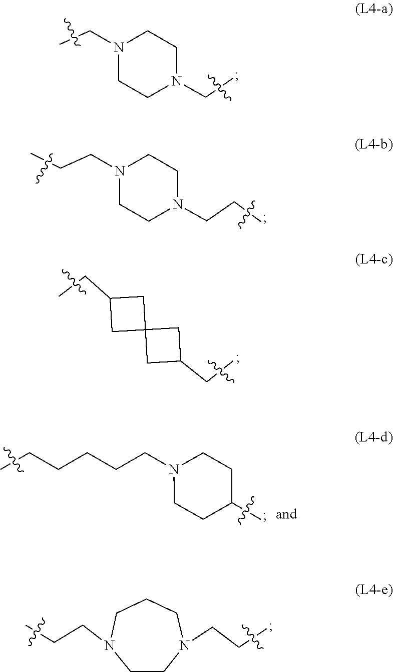

alkylene chains interrupted by amide, heterocyclene and/or aryl groups, examples of which include:

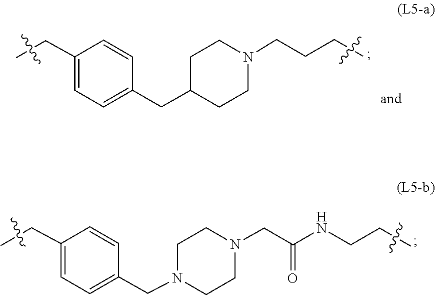

alkylene chains interrupted by heterocyclene and aryl groups, and a heteroatom, examples of which include:

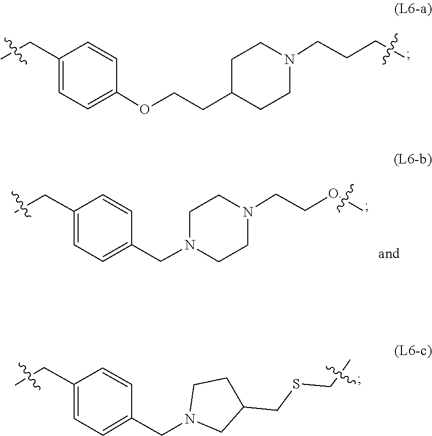

and
alkylene chains interrupted by and/or terminating in a heteroatom such as N, O or B, e.g.,

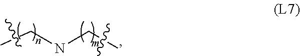

wherein each n is independently an integer of 1-10, e.g., 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10, and 1, 2, 3, 4, 5, 6, 7, 8, 9 and 10, and R is H or C₁ to C₄ alkyl, an example of which is

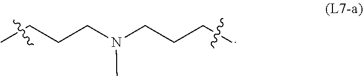

In some embodiments, the linker may include a polyethylene glycol (PEG) chain which may terminate at either or both termini with at least one of —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR¹)—, —N(R′)C(NR¹)—, —C(NR¹)N(R′)—, —N(R′)C(NR¹)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃₋₁₂ carbocyclene, 3- to 12-membered heterocyclene, 5- to 12-membered heteroarylene or any combination thereof, wherein R′ is H or C₁-C₆ alkyl, wherein the one or both terminating groups may be the same or different.

In some embodiments, the linker includes a polyethylene glycol chain having 1-10 PEG units. In some embodiments, the linker includes a polyethylene glycol chain having 1-6 PEG units.

Examples of linkers that include a polyethylene glycol chain include:

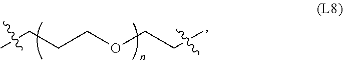

wherein n is an integer of 2-10, examples of which include:

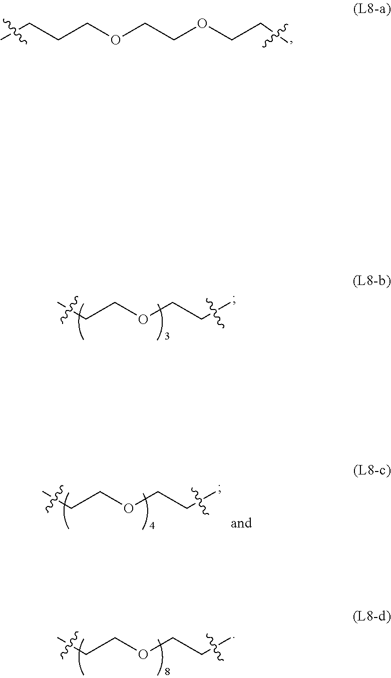

In some embodiments, the linker containing a polyethylene glycol chain may terminate in a functional group, examples of which are as follows:

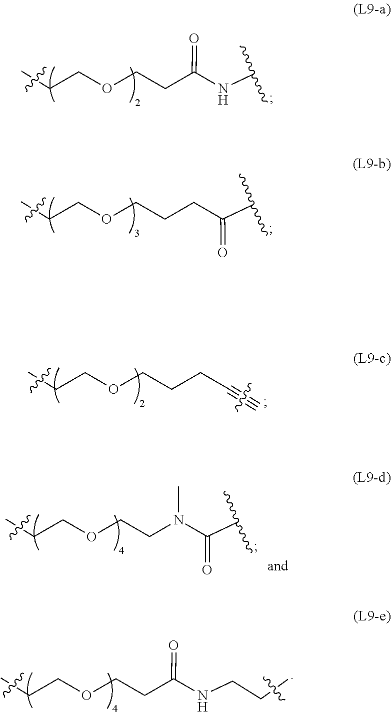

In some embodiments, the bispecific compound of formula (I) includes a linker that is represented by any one of the following structures:

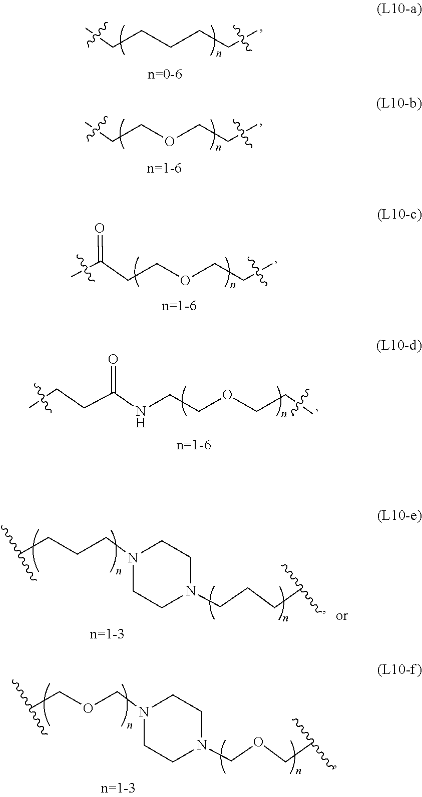

In some embodiments, the bispecific compound of formula (I) includes a linker that is represented by any one of the following structures:

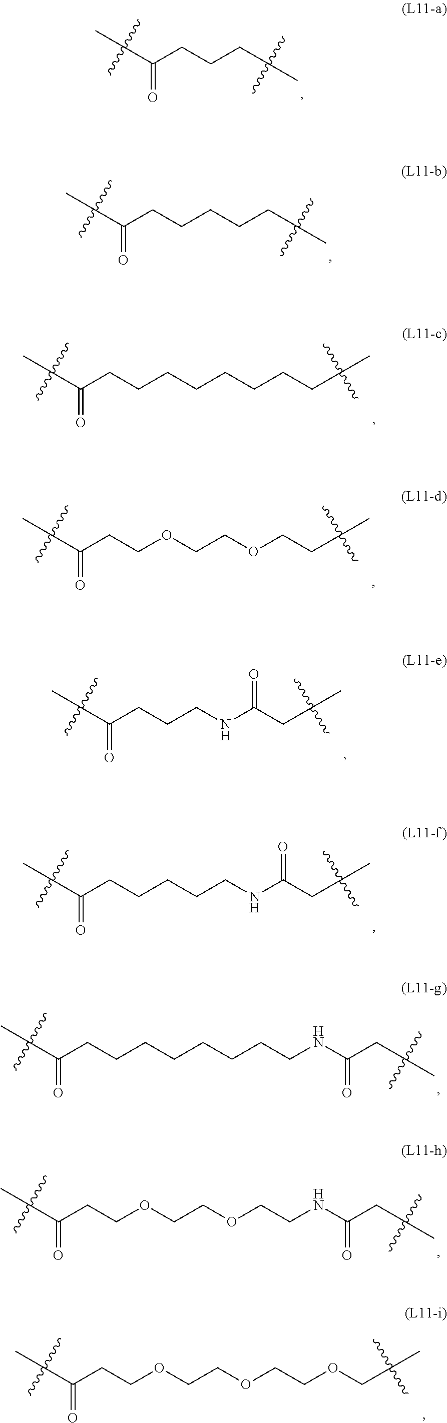

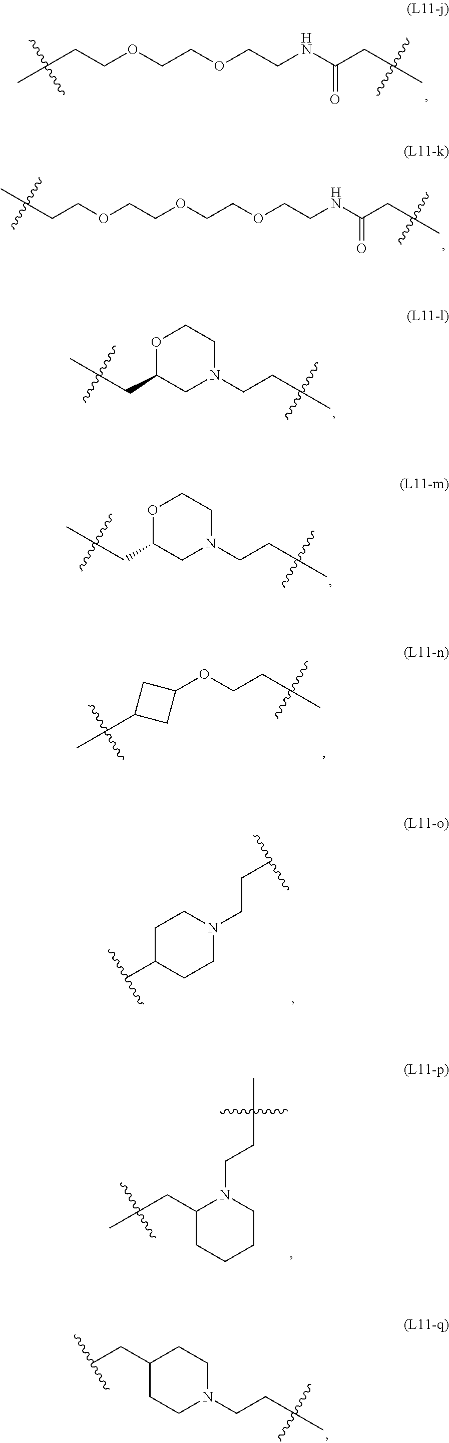

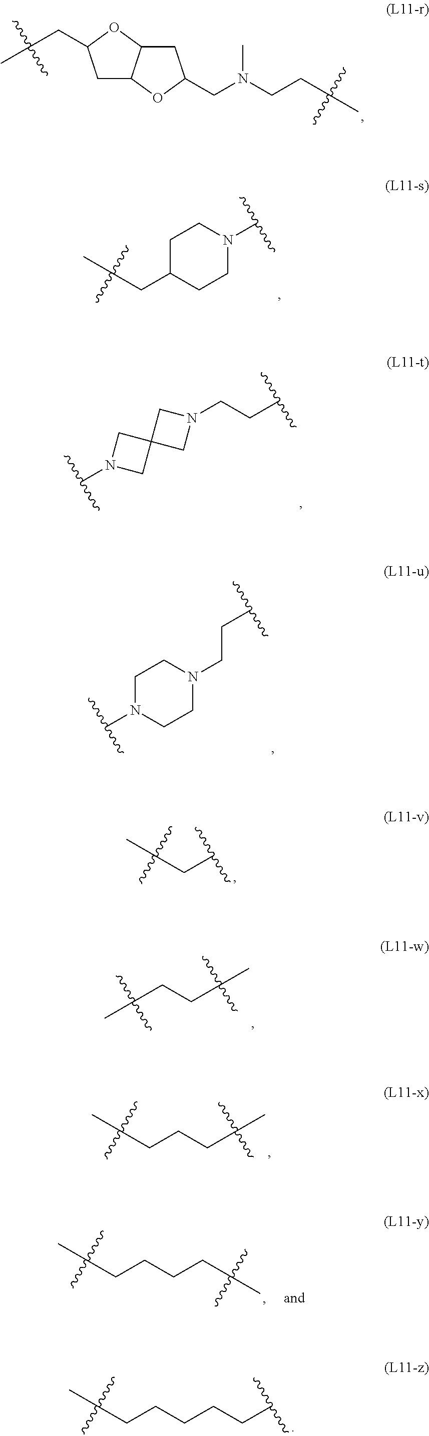

Therefore, in some embodiments, the bispecific compounds of the present invention are represented by any one of the following structures:

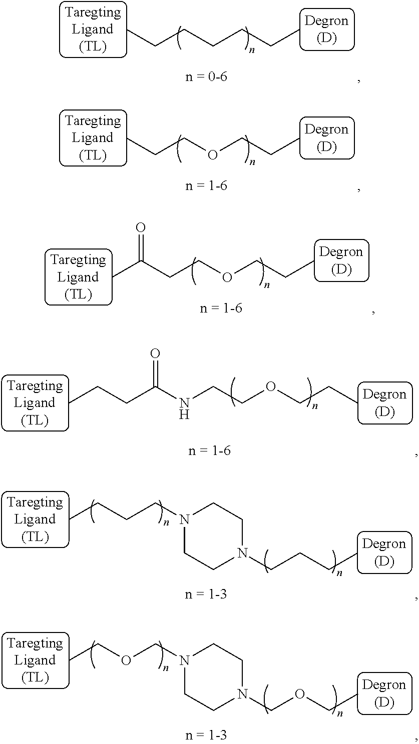

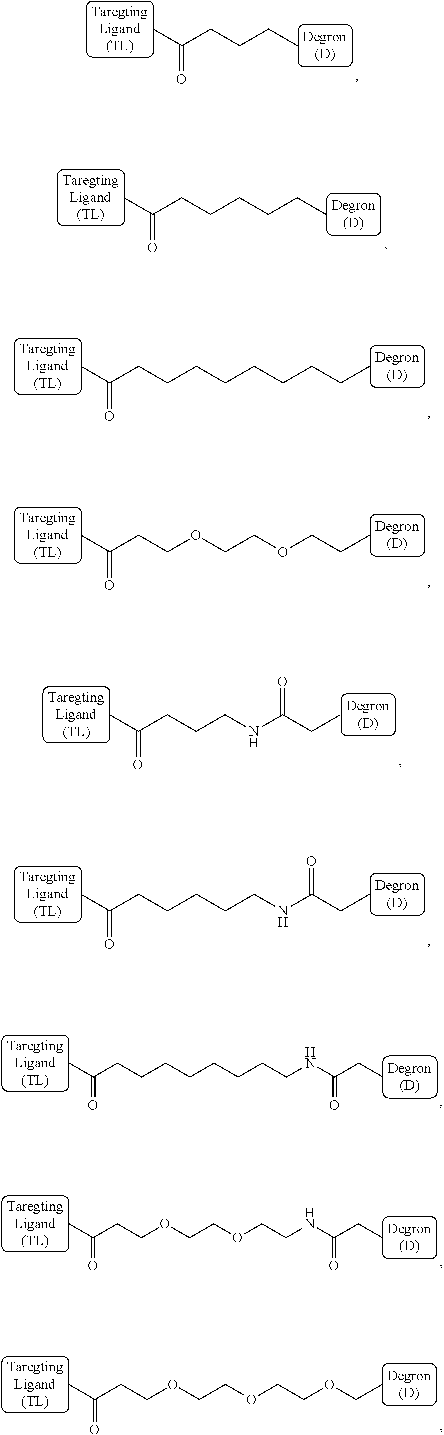

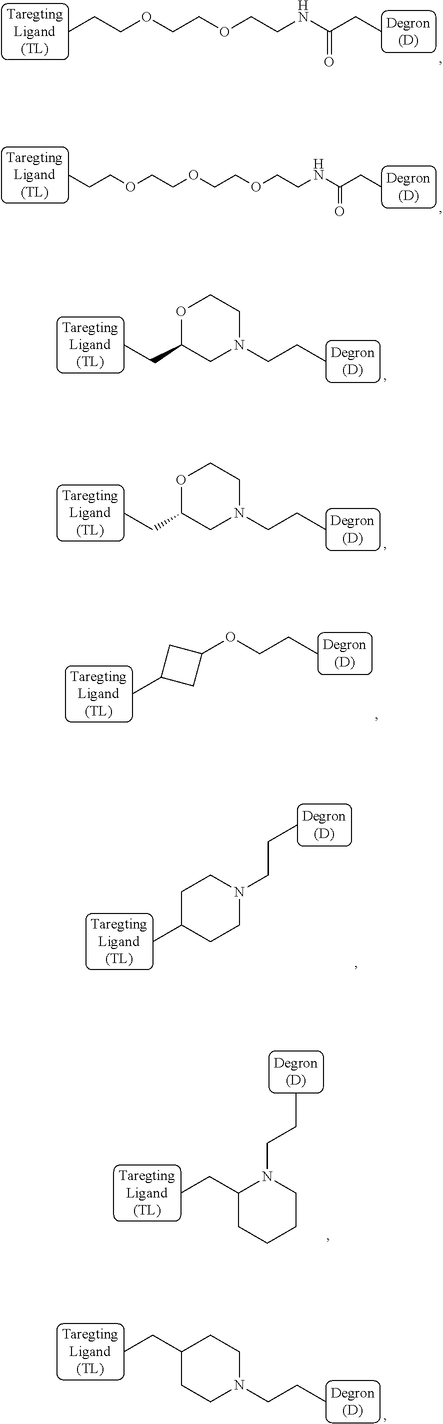

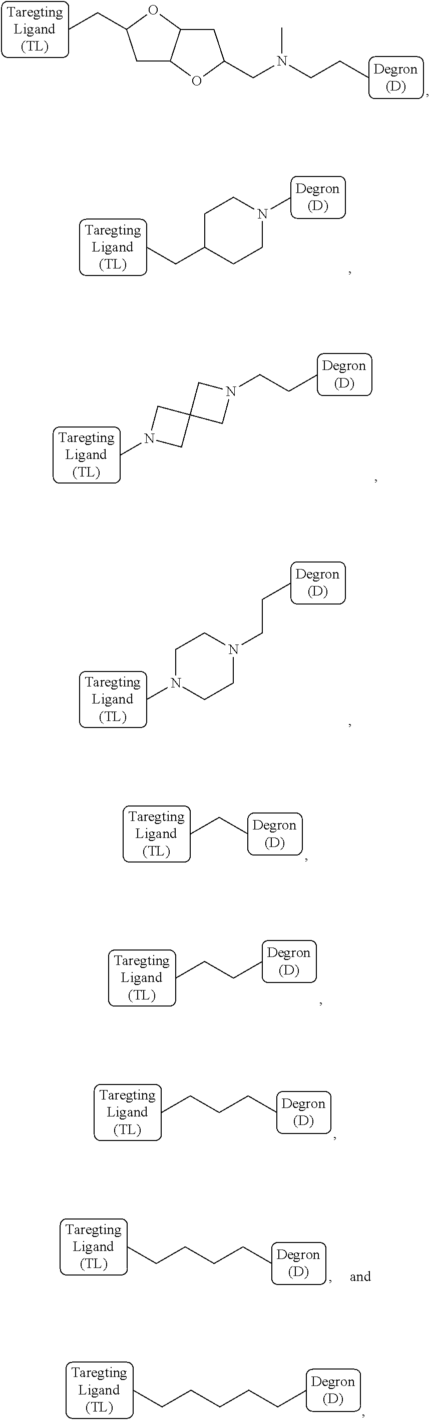

or a pharmaceutically acceptable salt or stereoisomer thereof.

In some embodiments, the bispecific compounds of the present invention are represented by any one of the following structures:

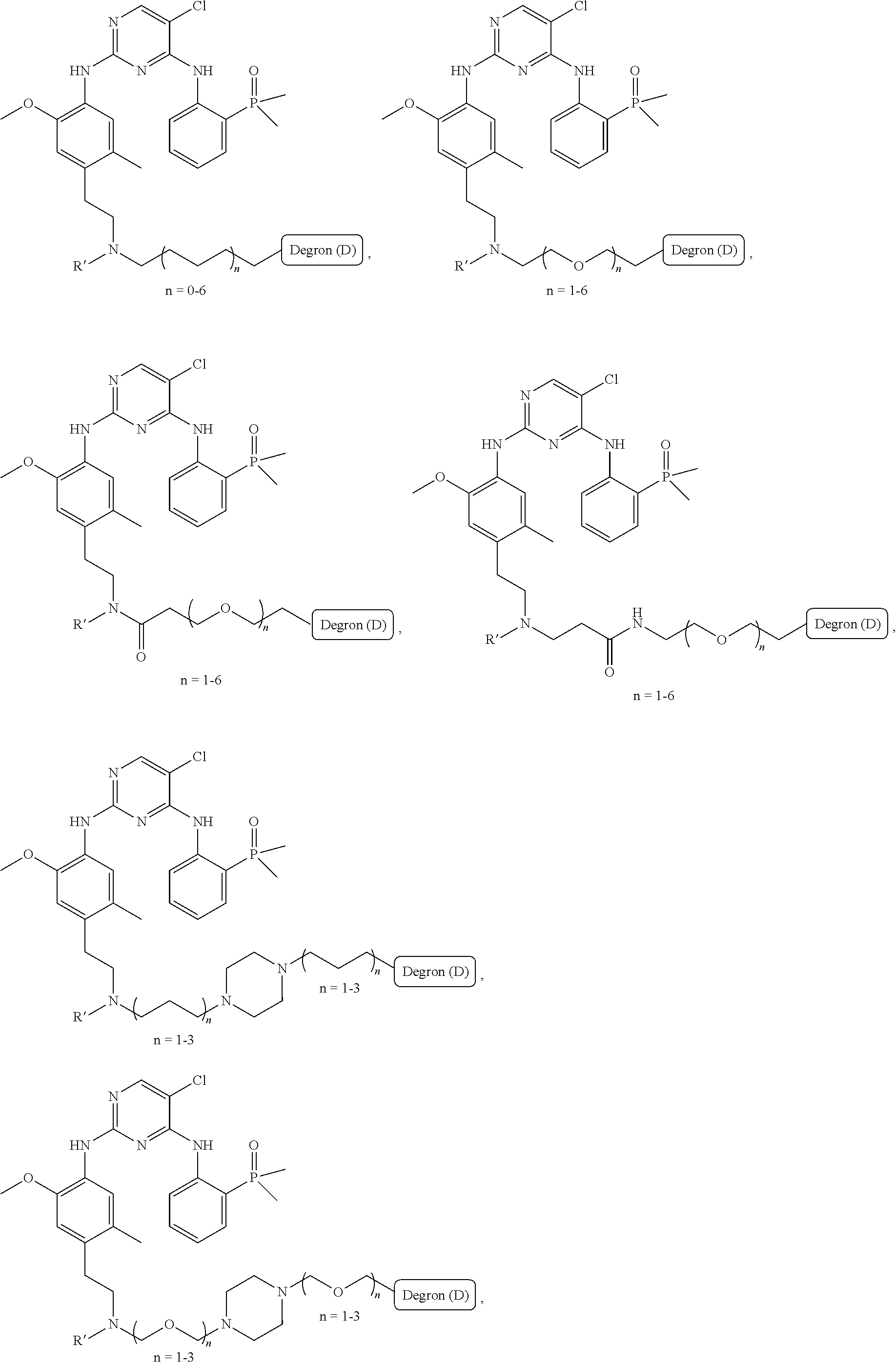

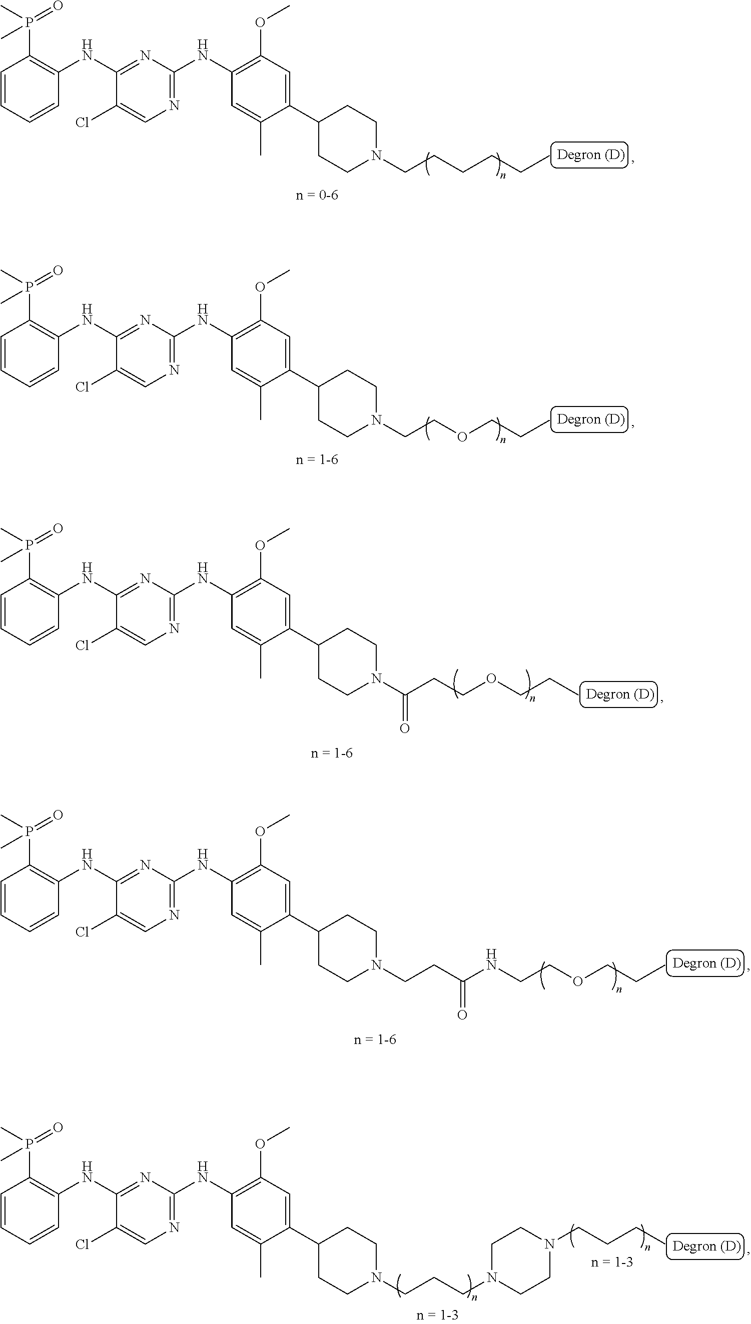

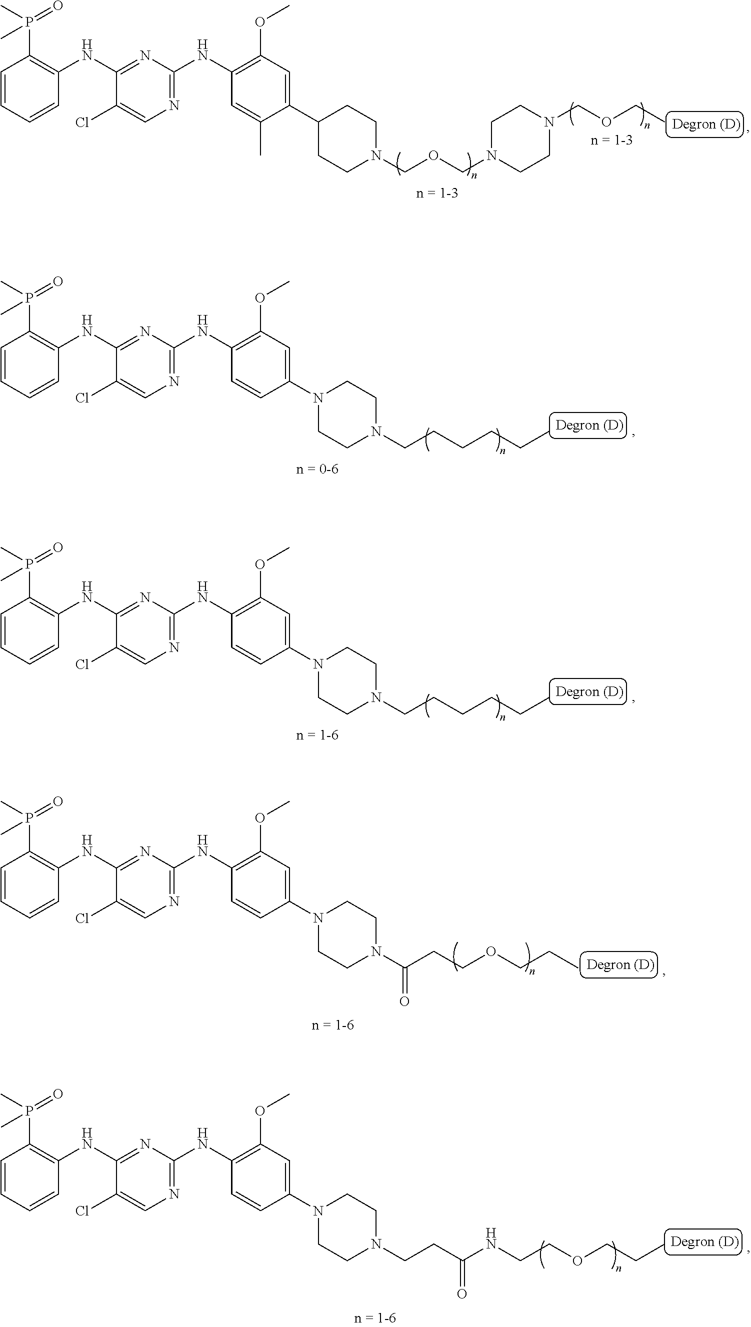

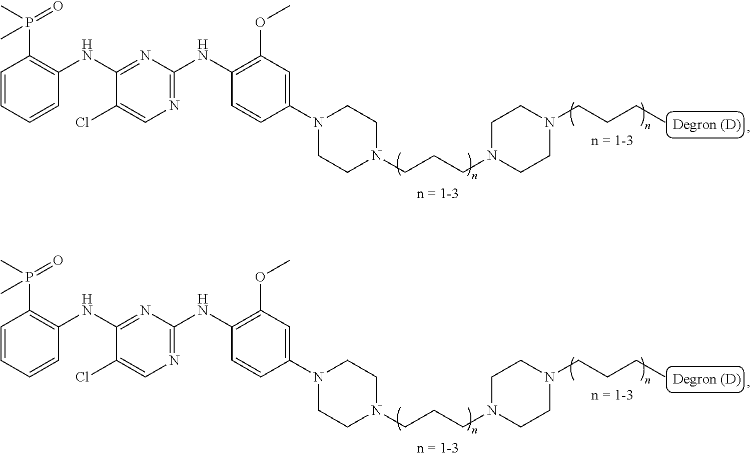

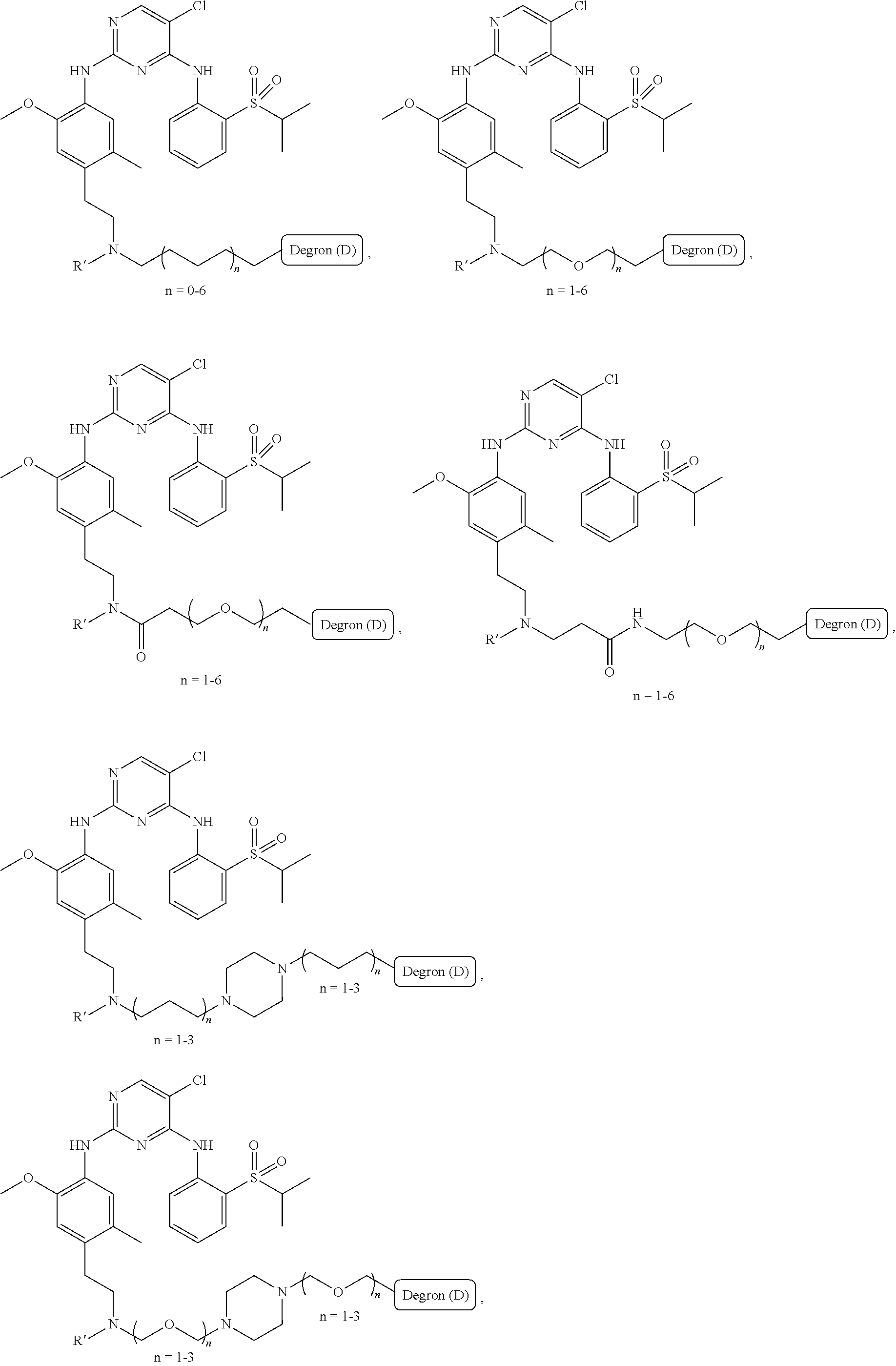

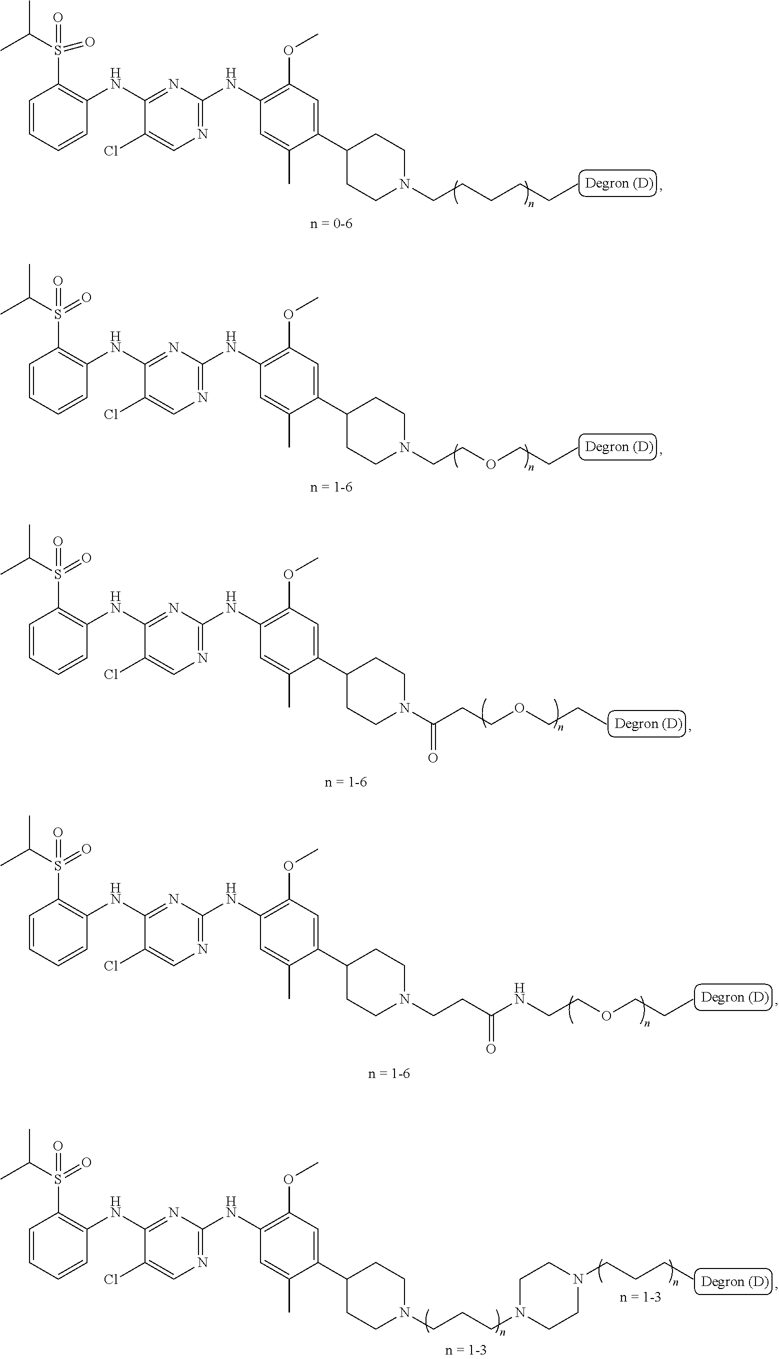

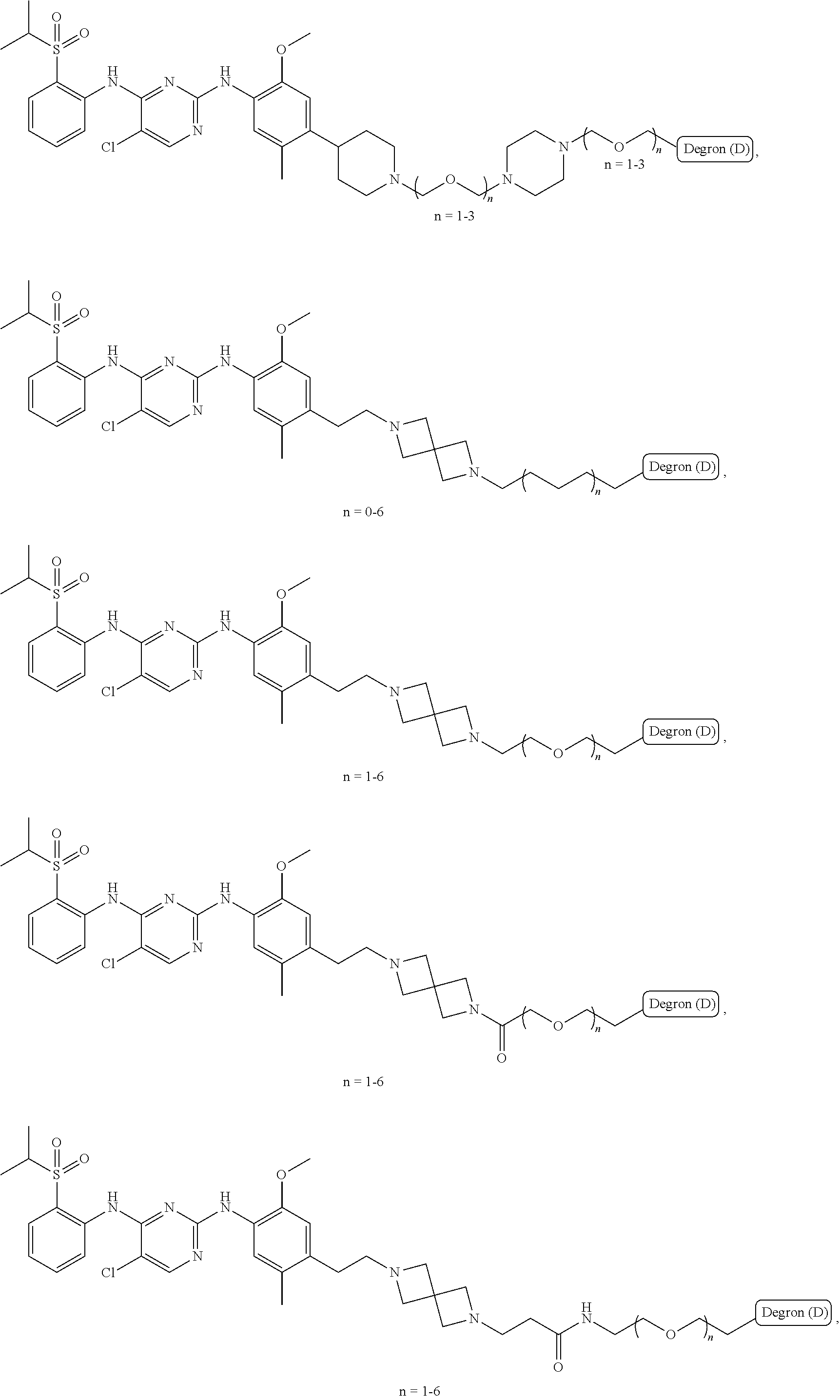

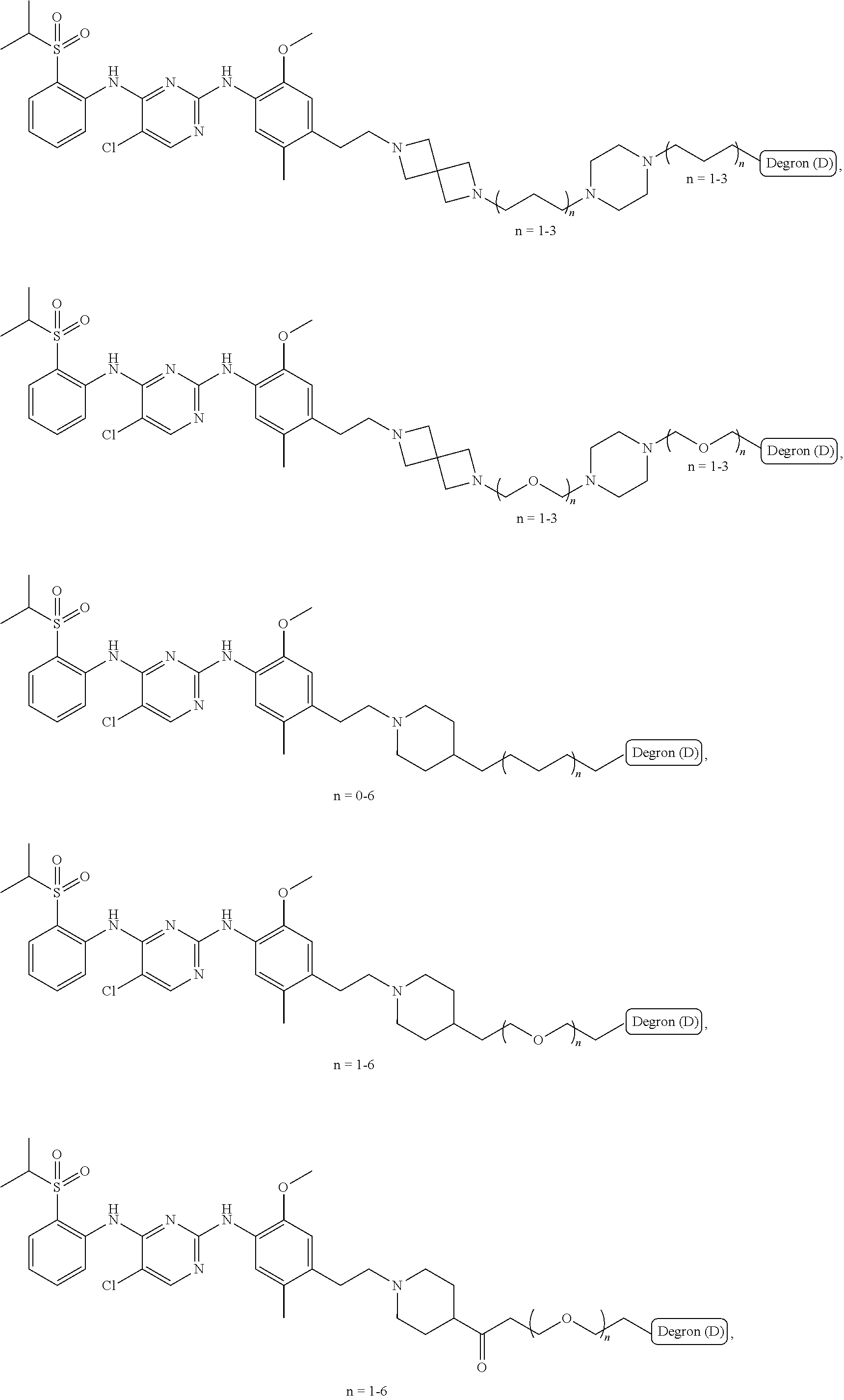

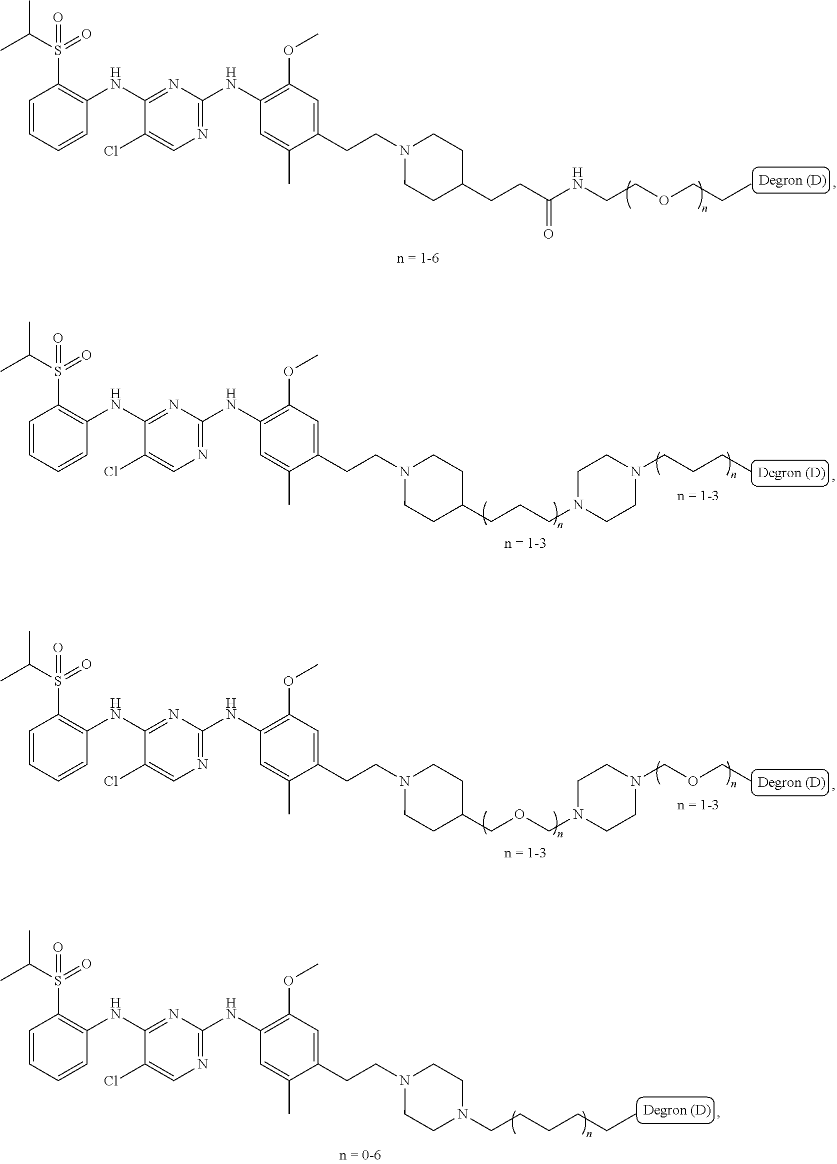

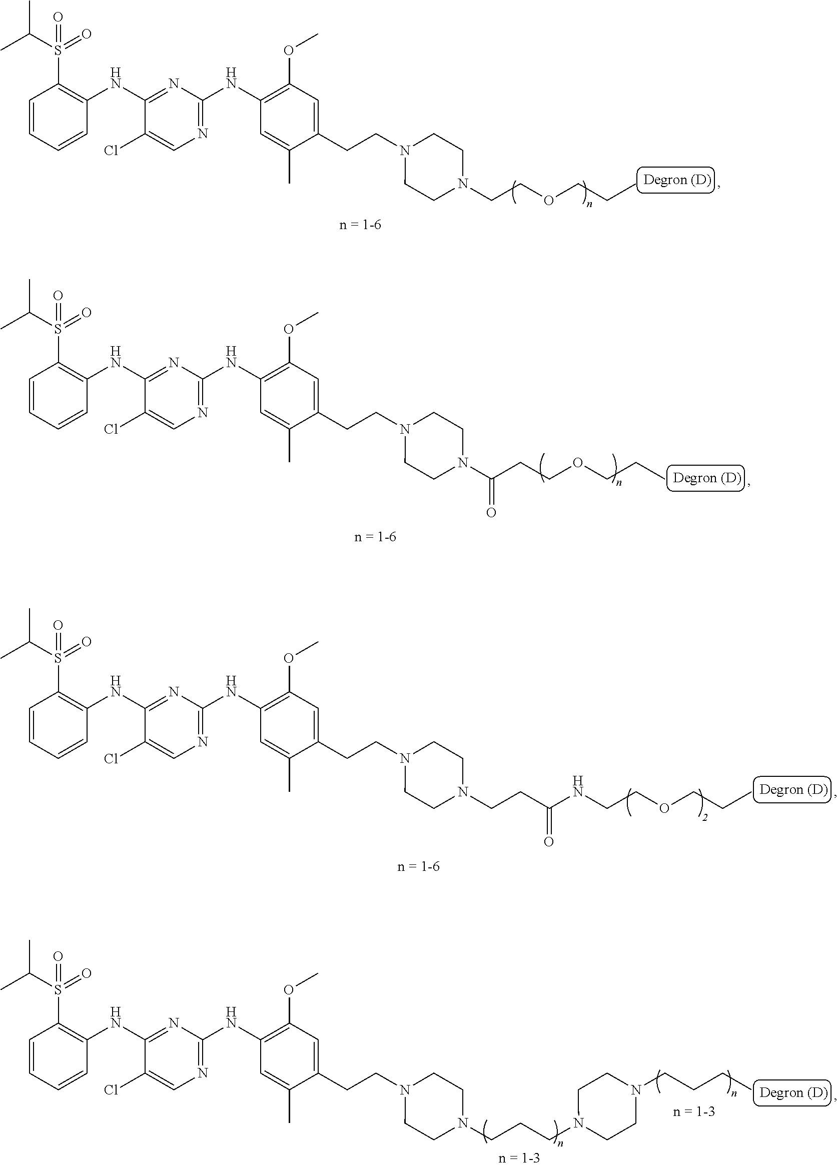

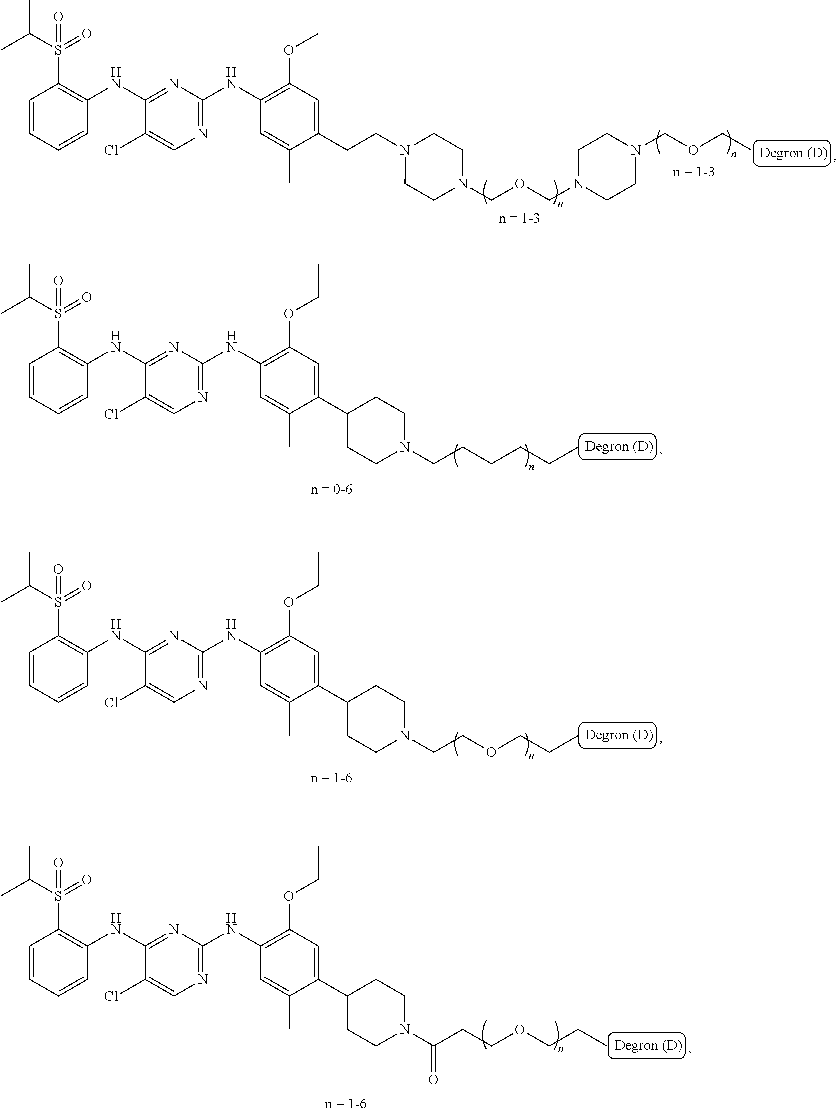

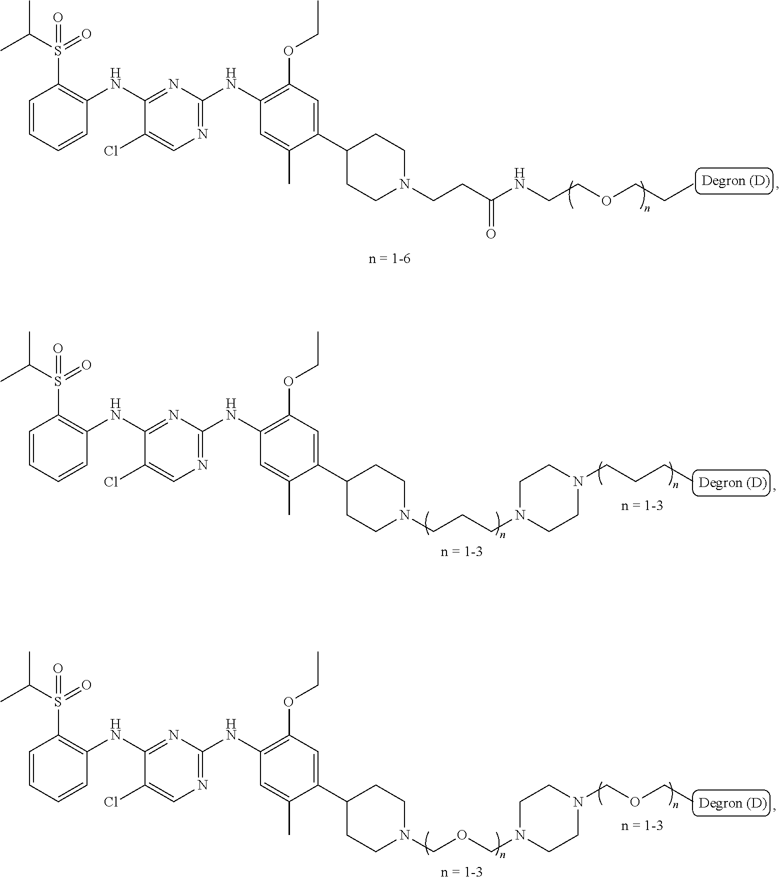

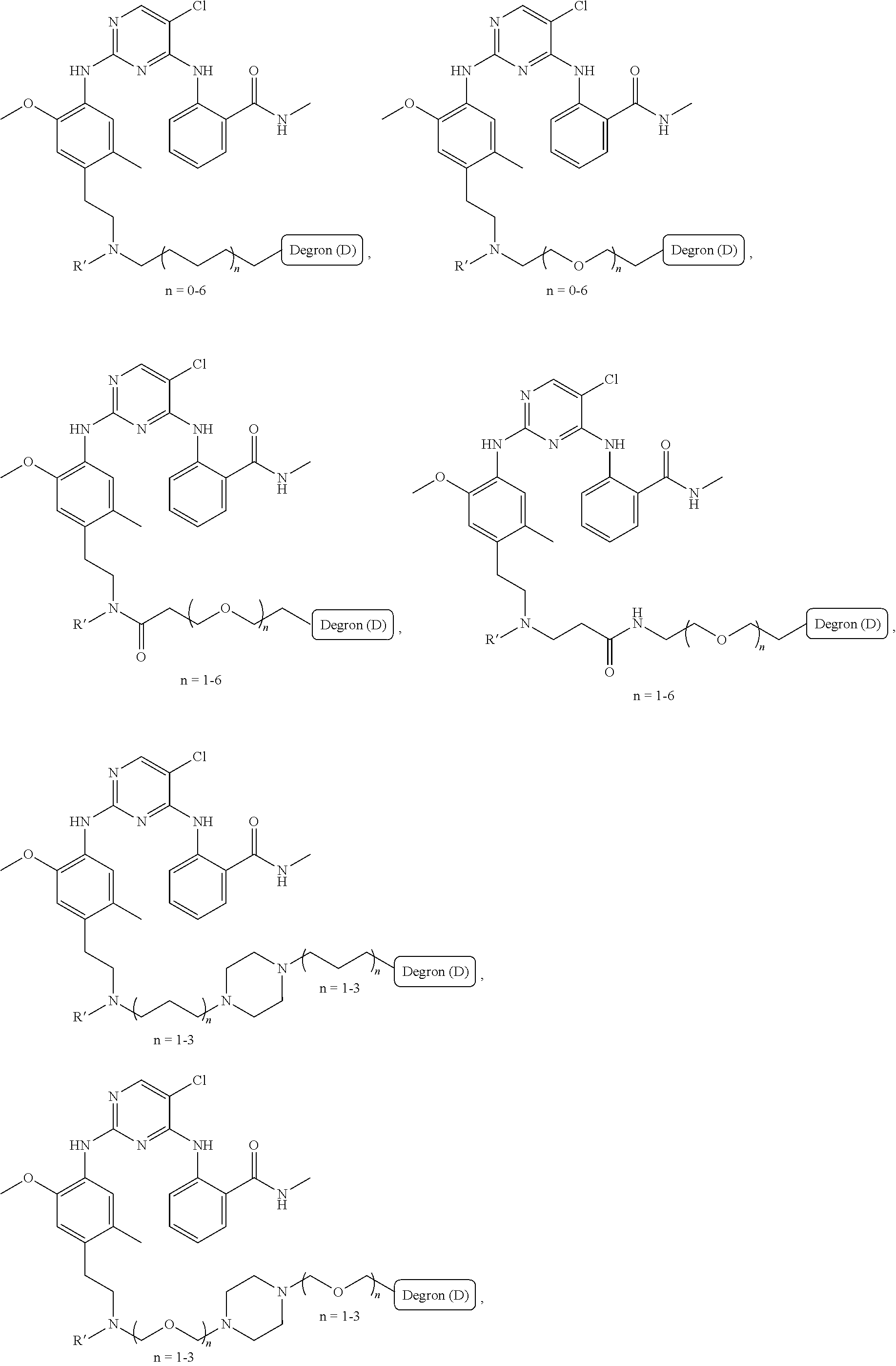

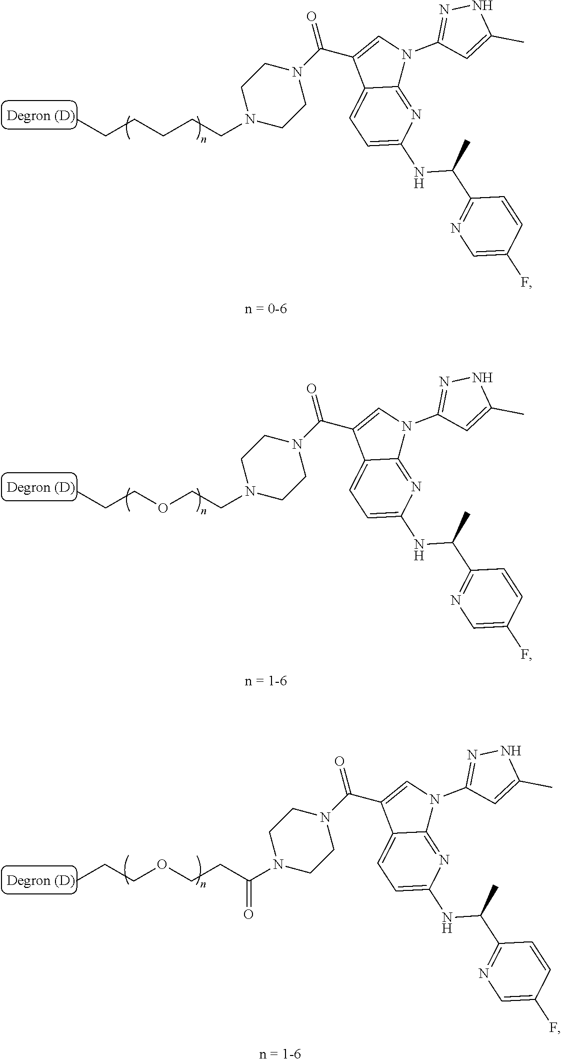

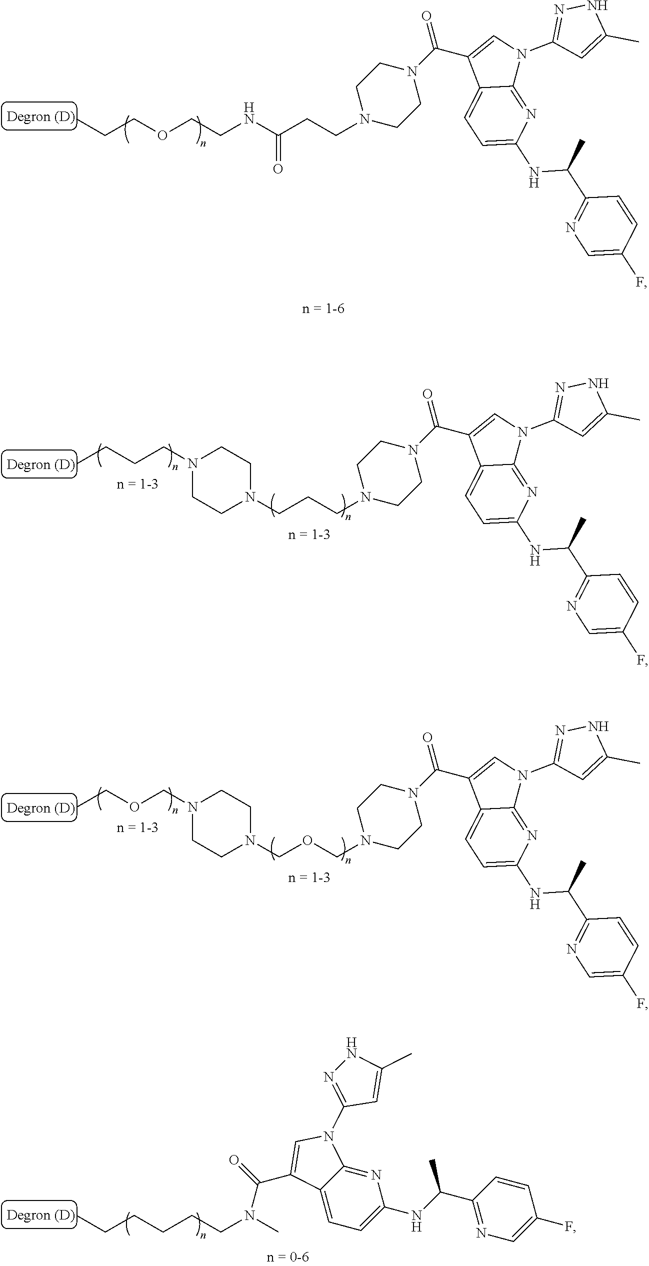

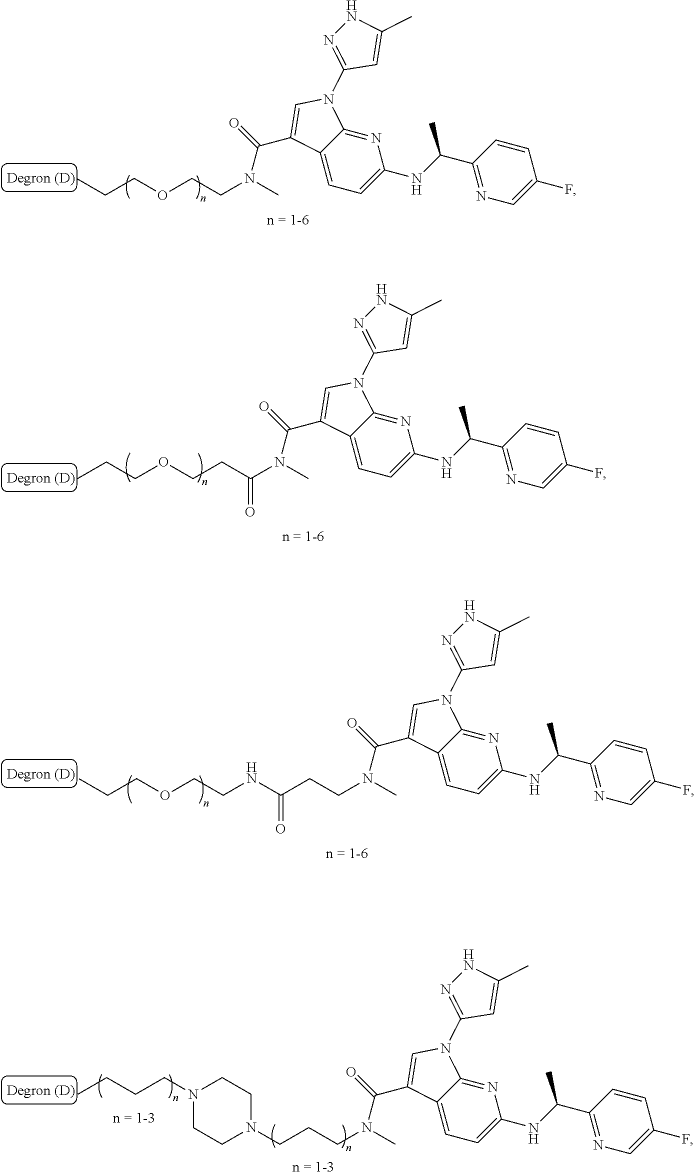

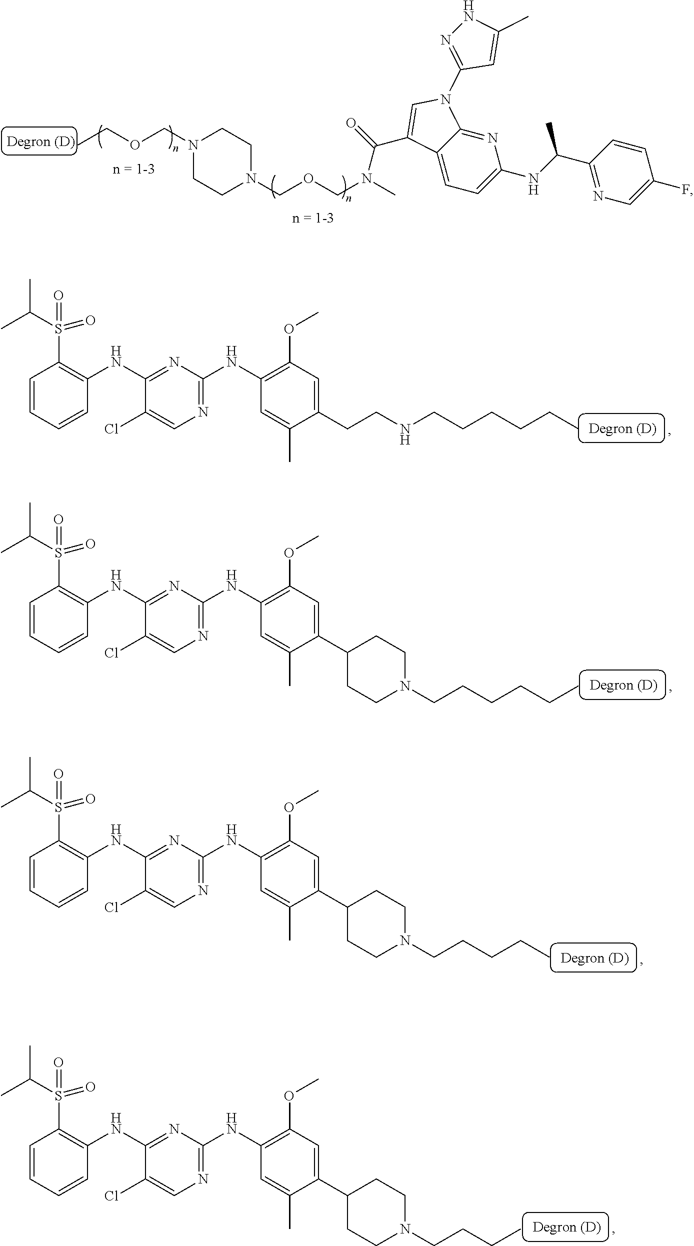

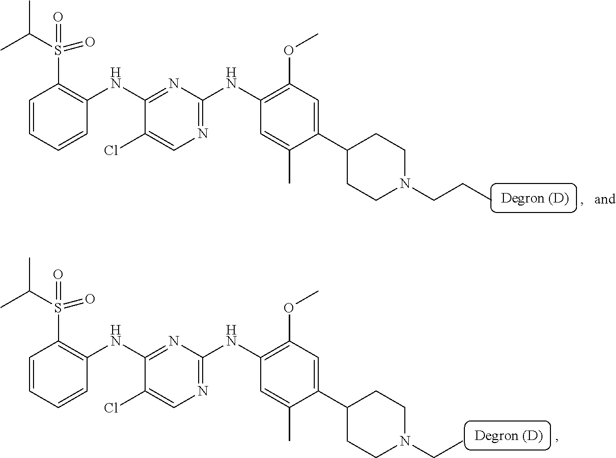

or a pharmaceutically acceptable salt or stereoisomer thereof, wherein R′ is H or Me and wherein in any of the bispecific compounds disclosed above that contain linkers defined by more than one “n”, each “n” may be the same or different.

### Degrons

The Ubiquitin-Proteasome Pathway (UPP) is a critical cellular pathway that regulates key regulator proteins and degrades misfolded or abnormal proteins. UPP is central to multiple cellular processes. The covalent attachment of ubiquitin to specific protein substrates is achieved through the action of E3 ubiquitin ligases. These ligases include over 500 different proteins and are categorized into multiple classes defined by the structural element of their E3 functional activity.

In some embodiments, the E3 ubiquitin ligase bound by the degron is cereblon (CRBN), and is represented by formula D1:

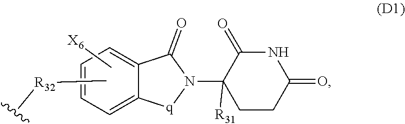

or a stereoisomer thereof
wherein,

### Q is CH₂, S, C═O, or

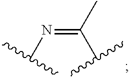 [SMILES: */N=C(\*)C]

R₃₁ is hydrogen or methyl;
R₃₂ is CH₂, NH, O, C≡C,

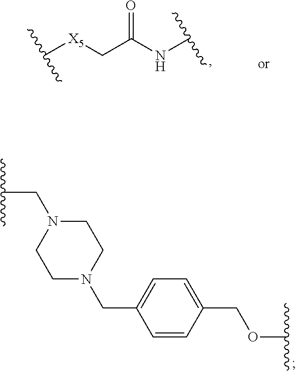

X₅ is absent, CH₂, NH, or O; and
X₆ is alkyl, halo, CN, CF₃, OCHF₂ or OCF₃.

In some embodiments, formula D1 is represented by any one of the following structures:

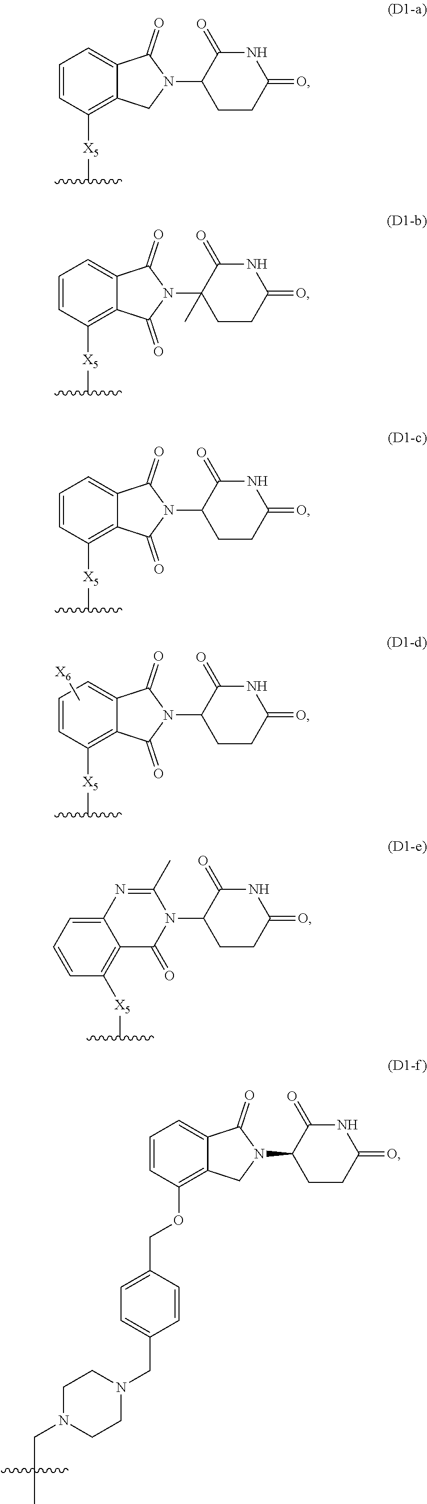

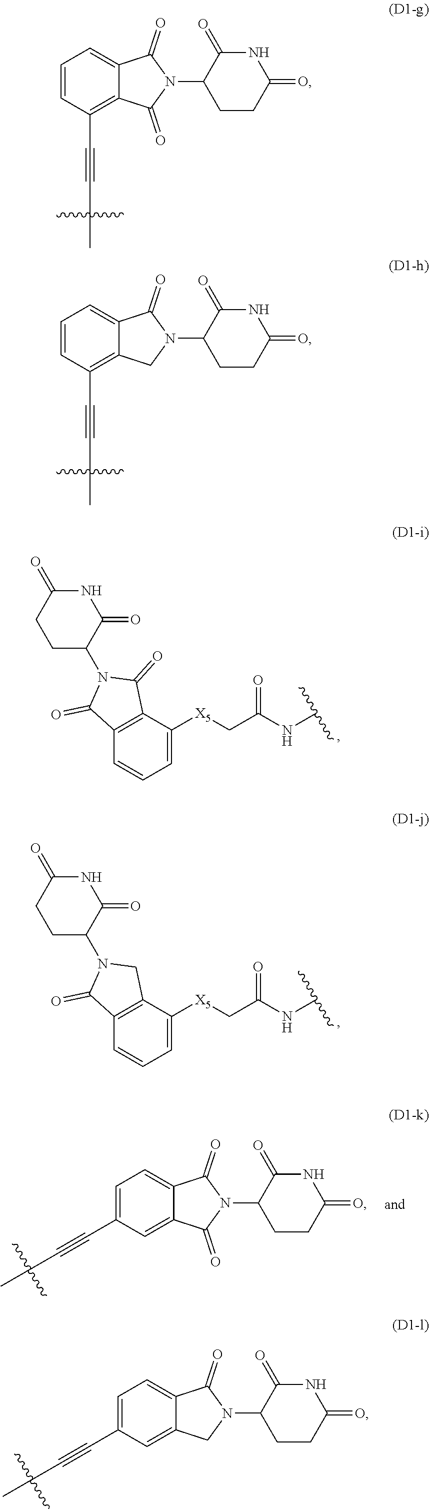

Therefore, in some embodiments, bispecific compounds of the present invention are represented by any one of the following structures:

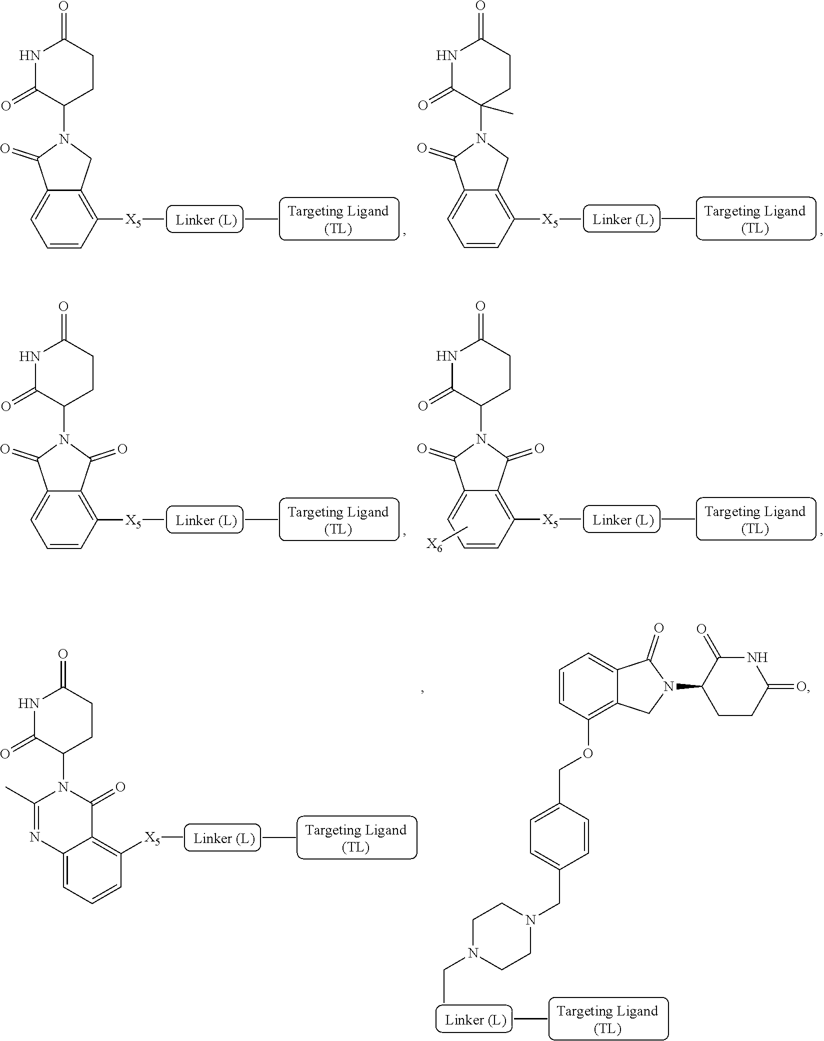

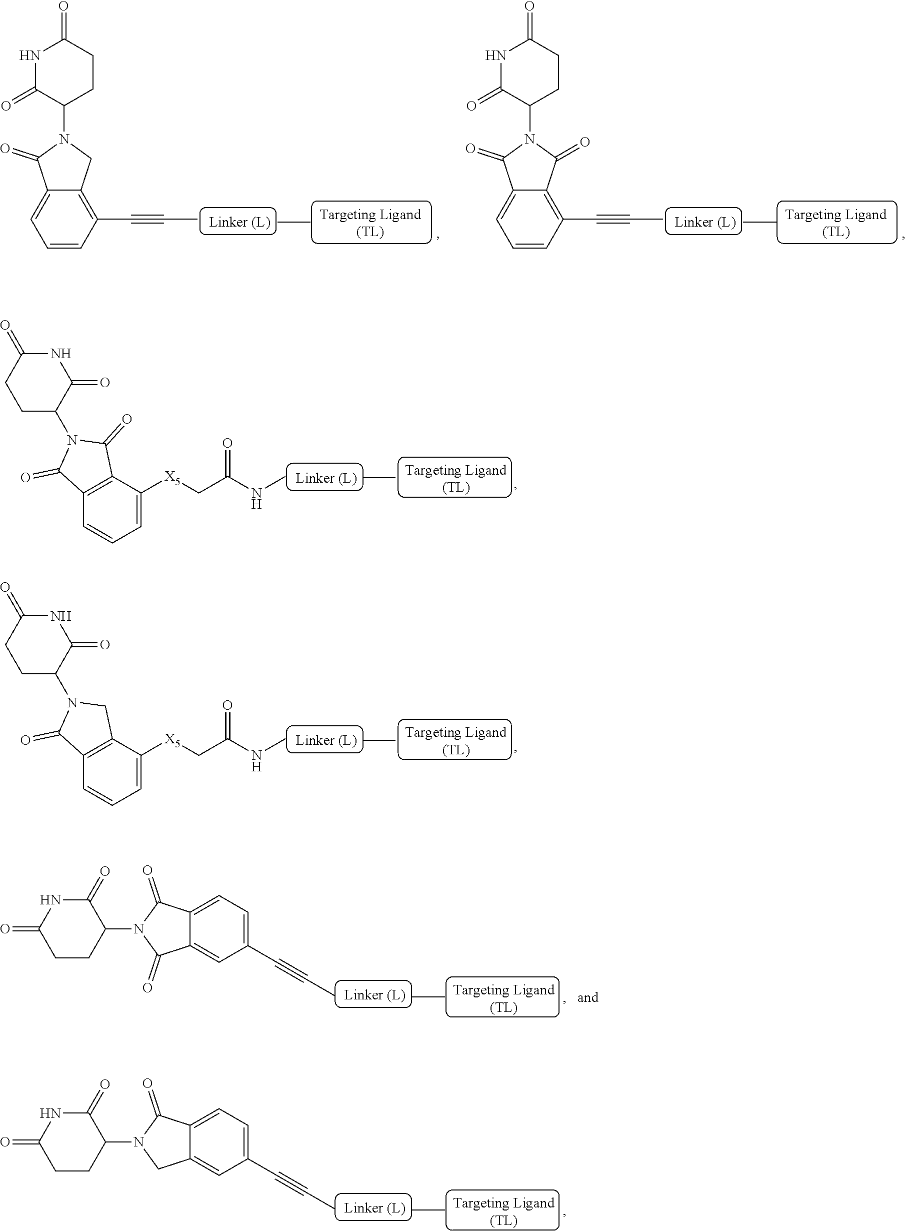

or a pharmaceutically acceptable salt or stereoisomer thereof.

In some embodiments, bispecific compounds of the present invention are represented by any one of the following structures:

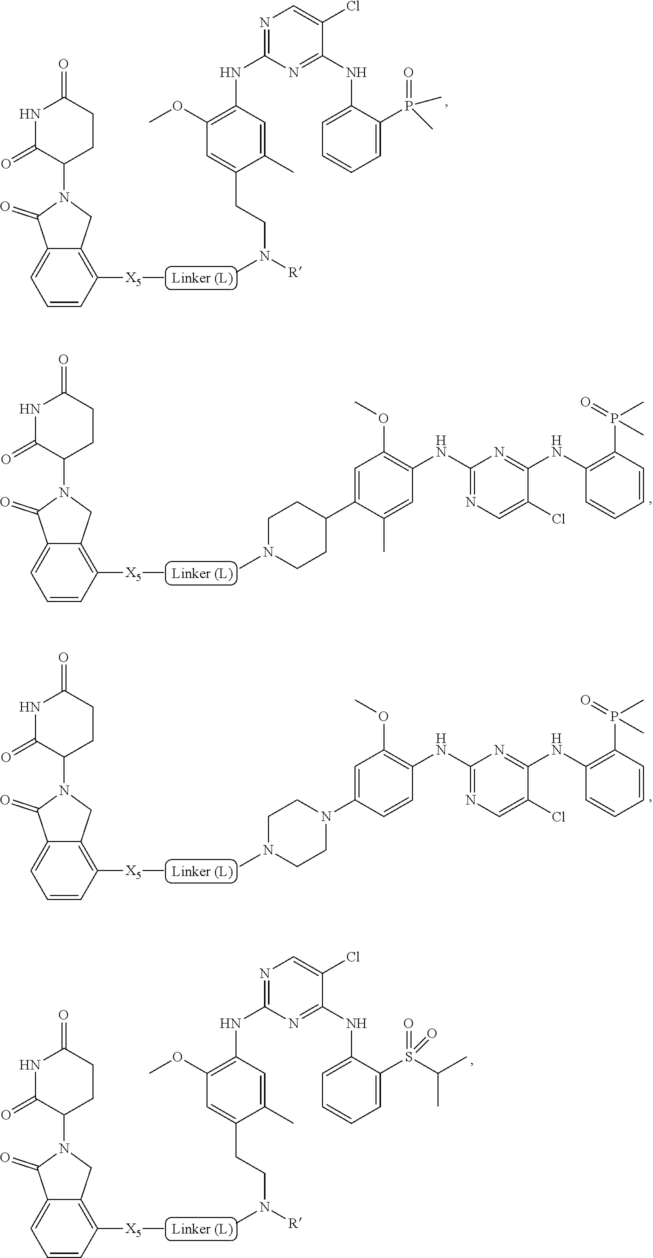

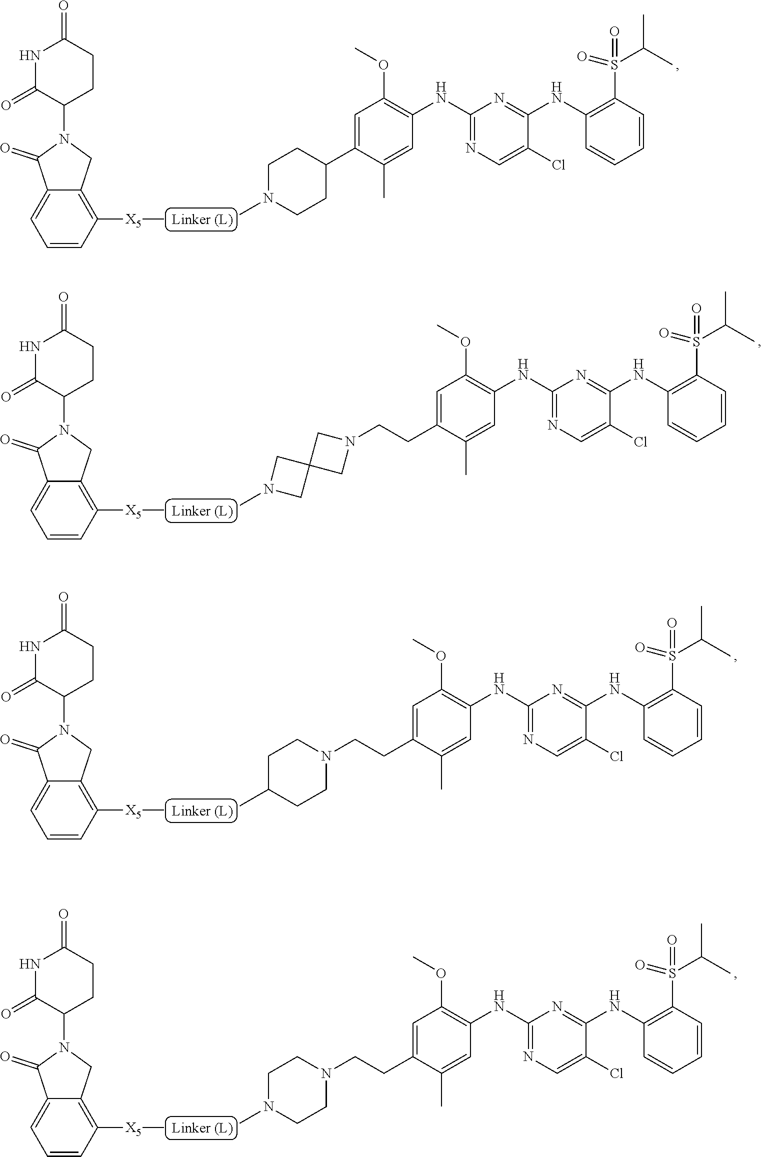

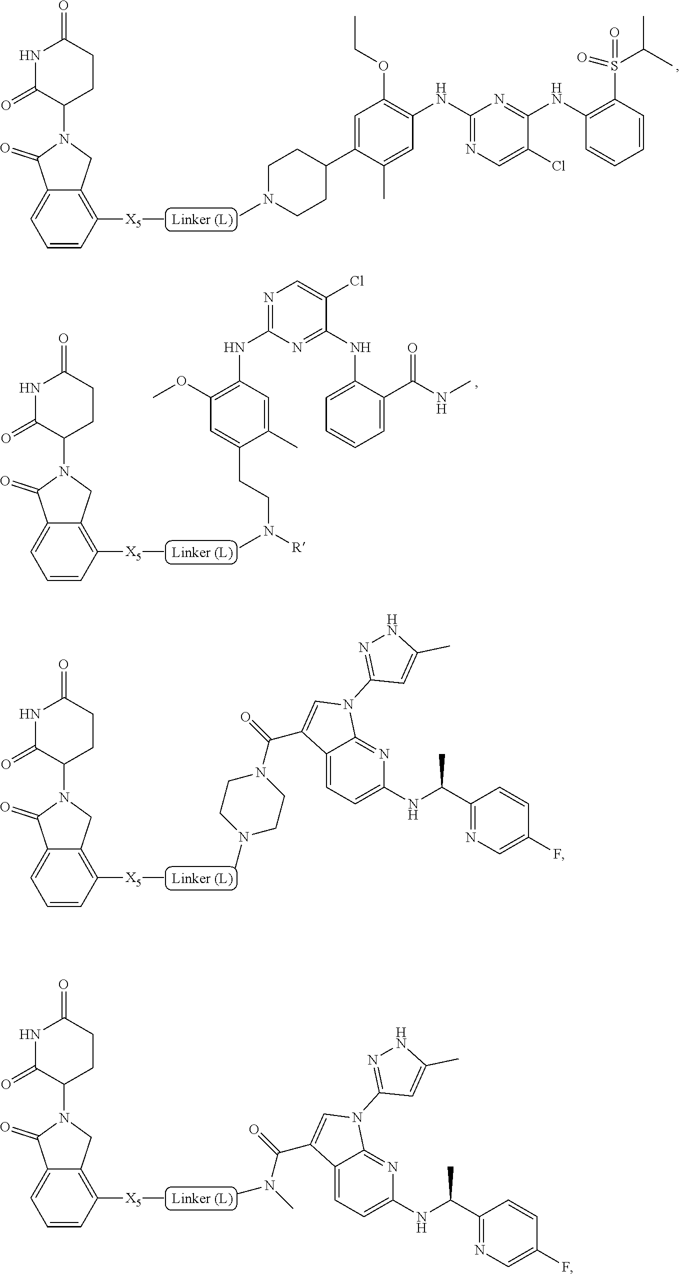

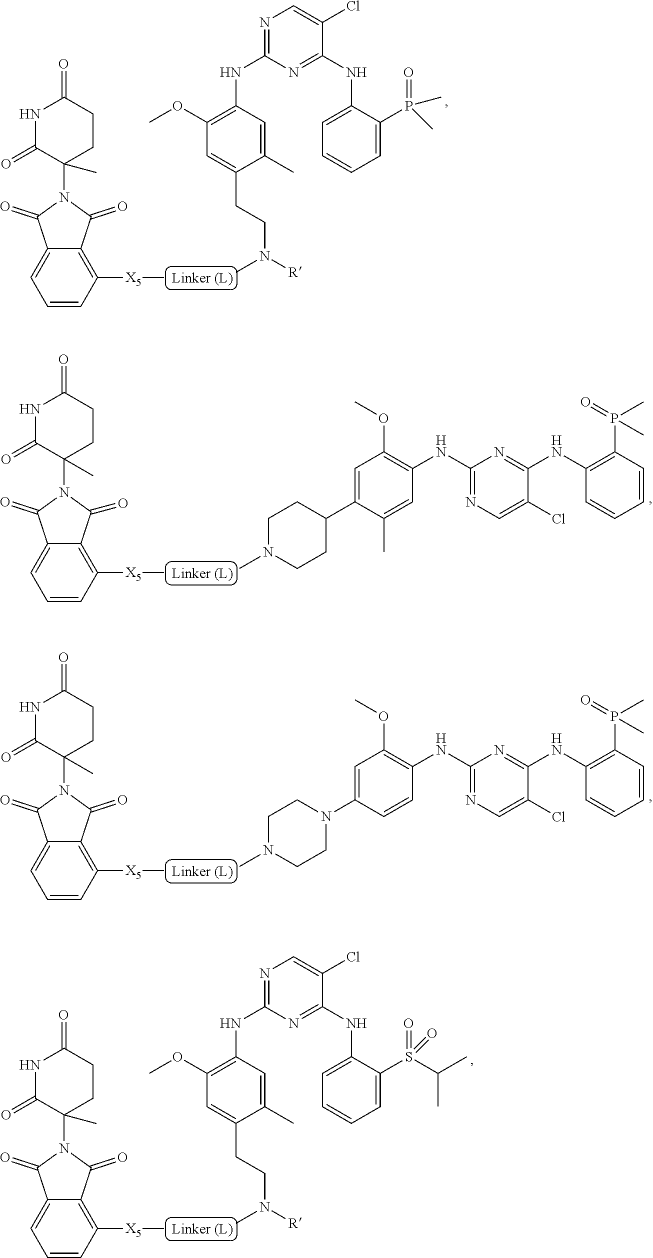

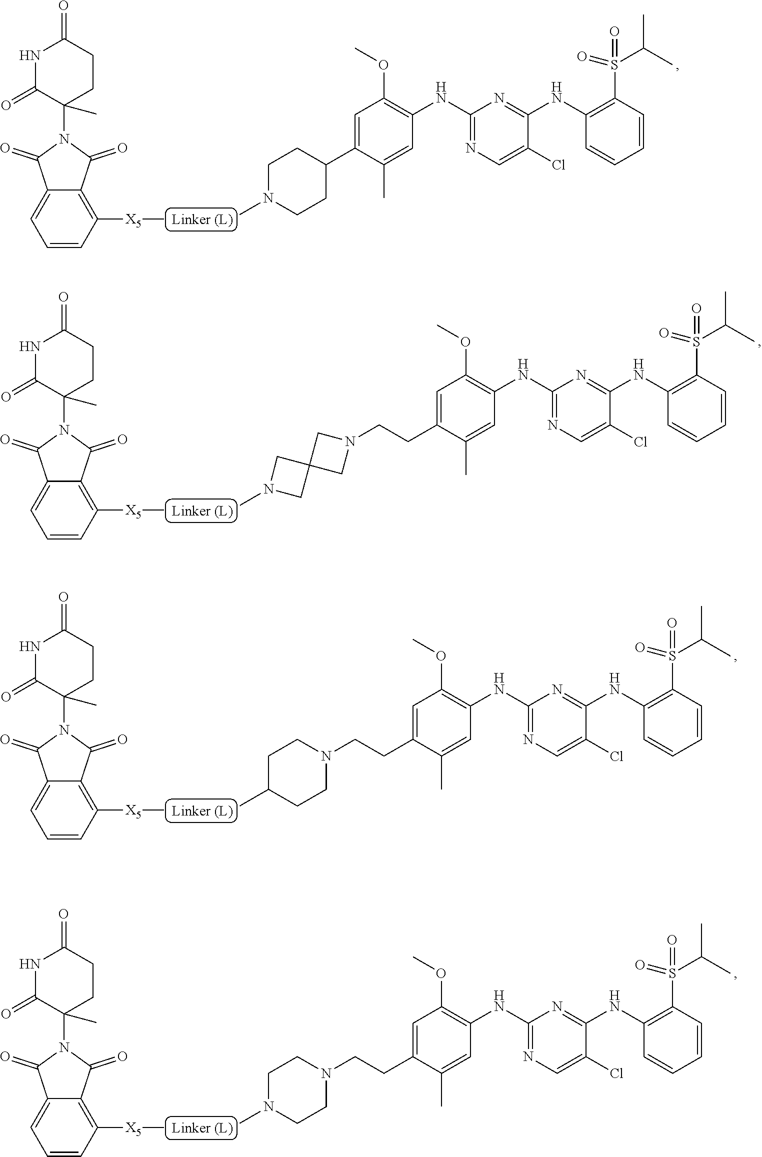

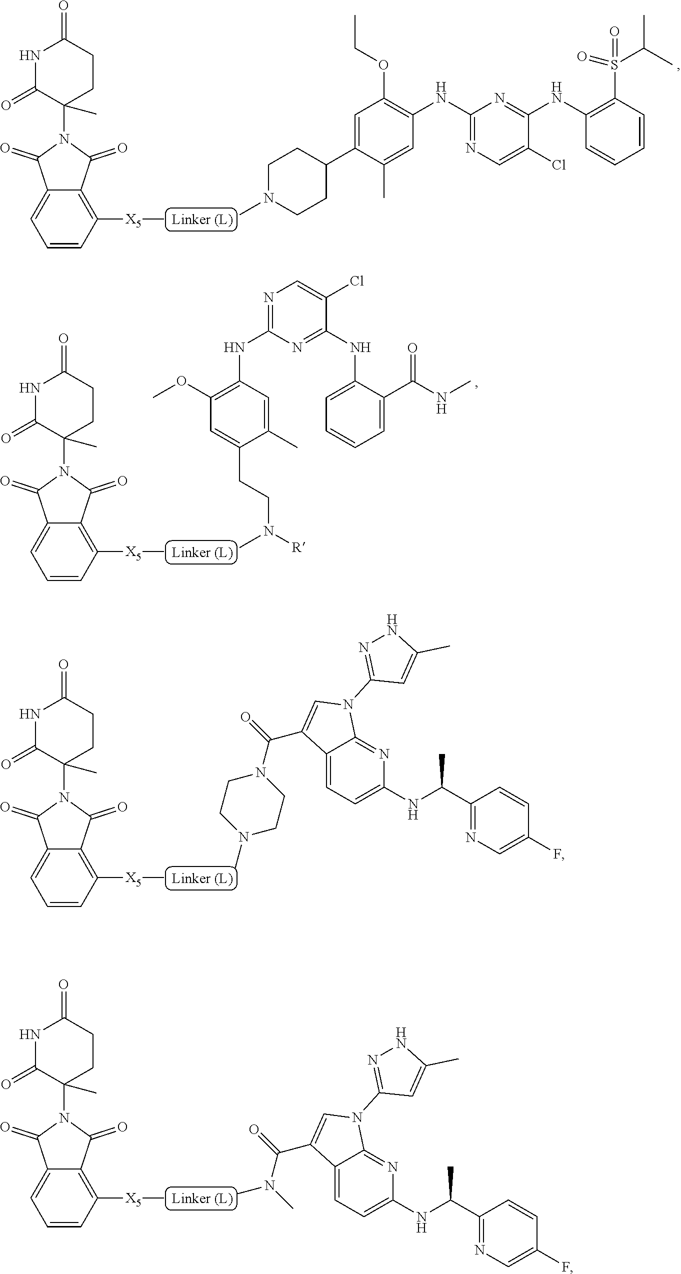

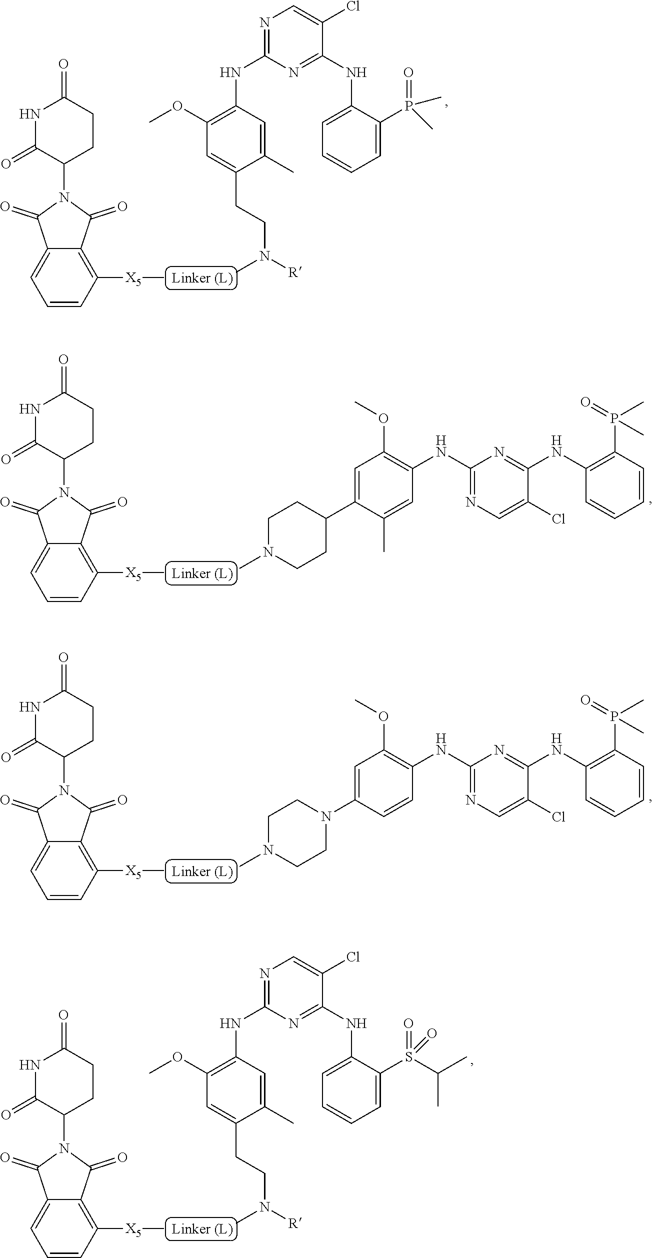

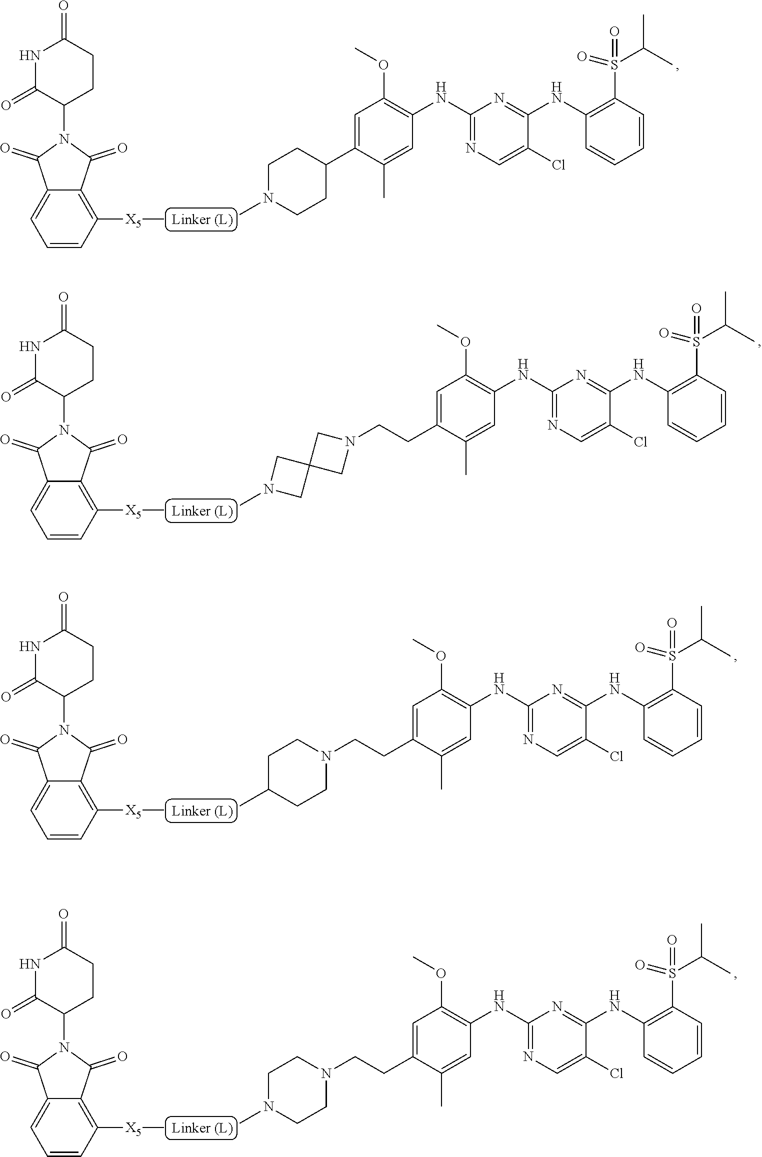

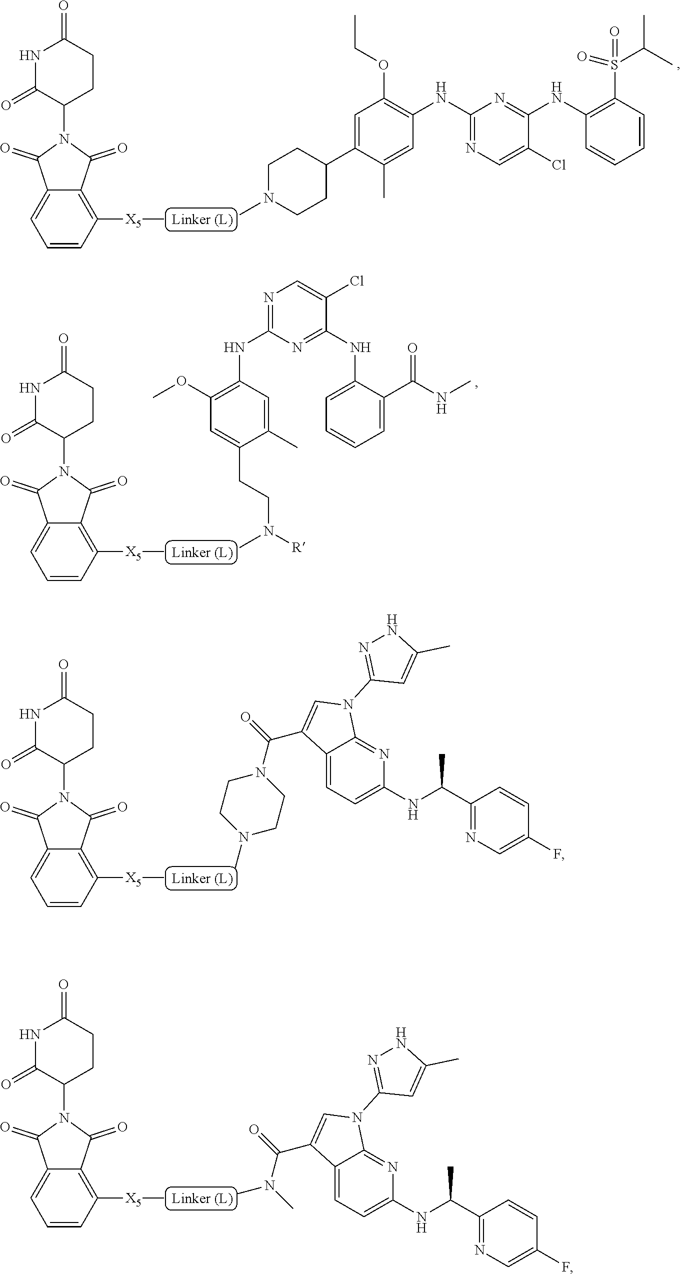

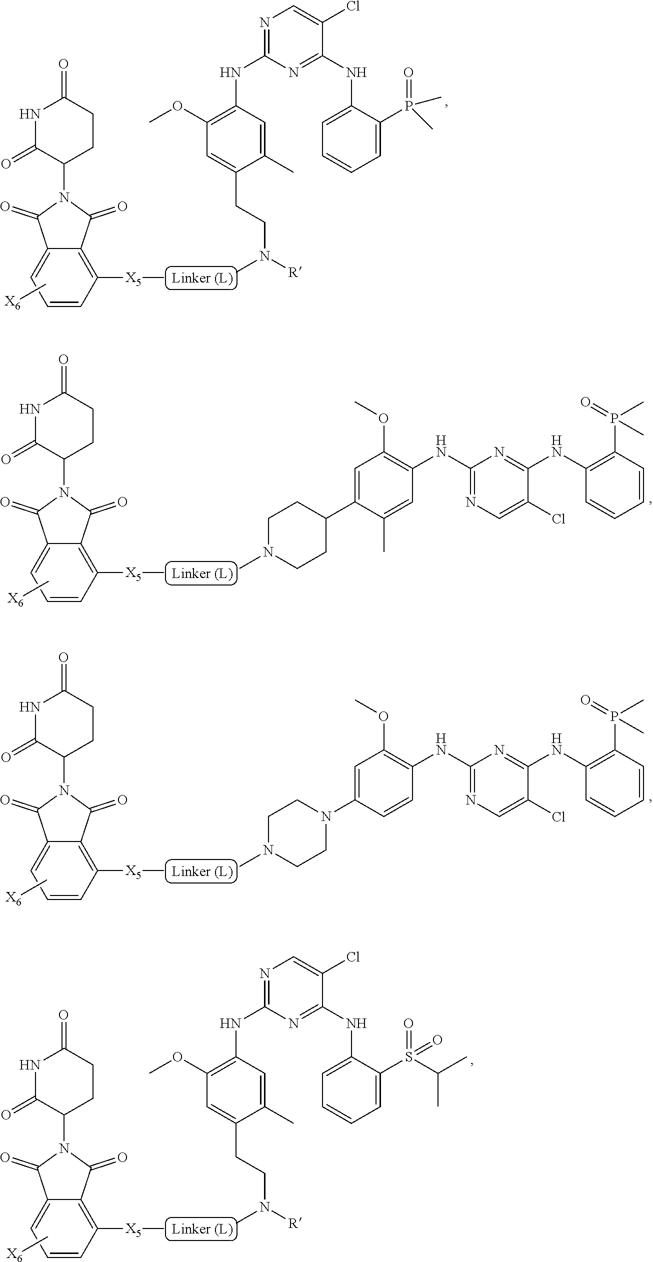

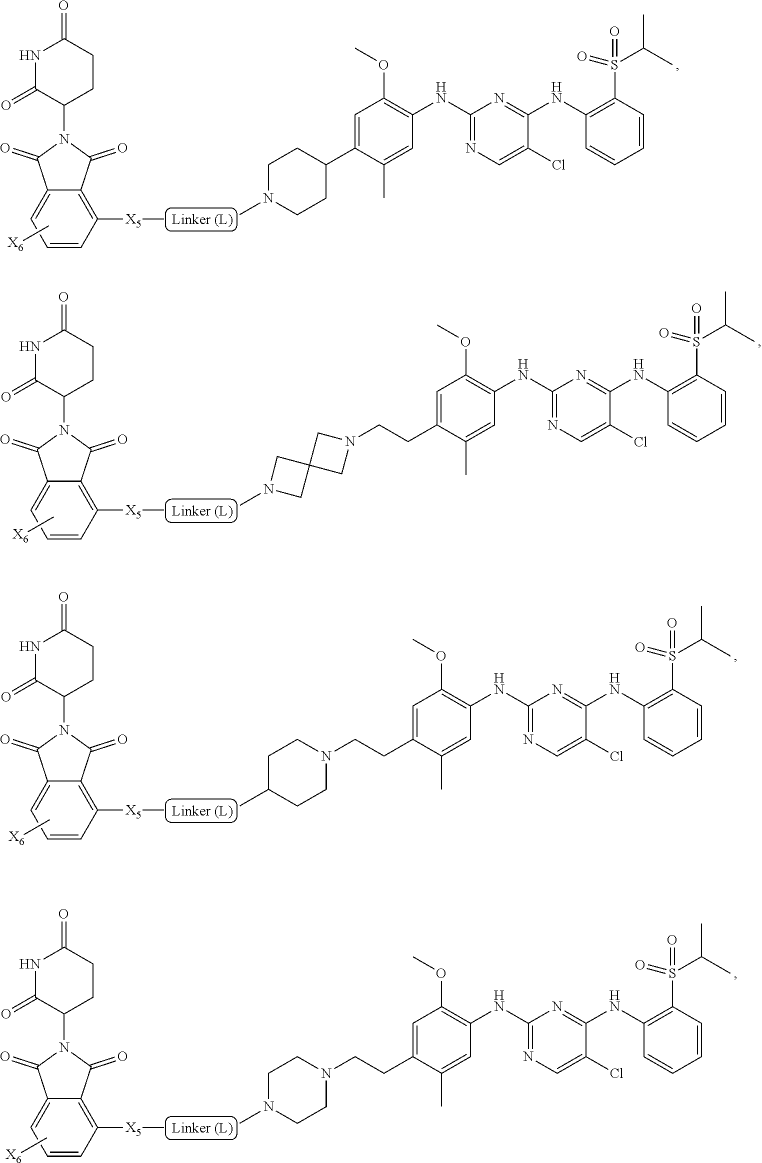

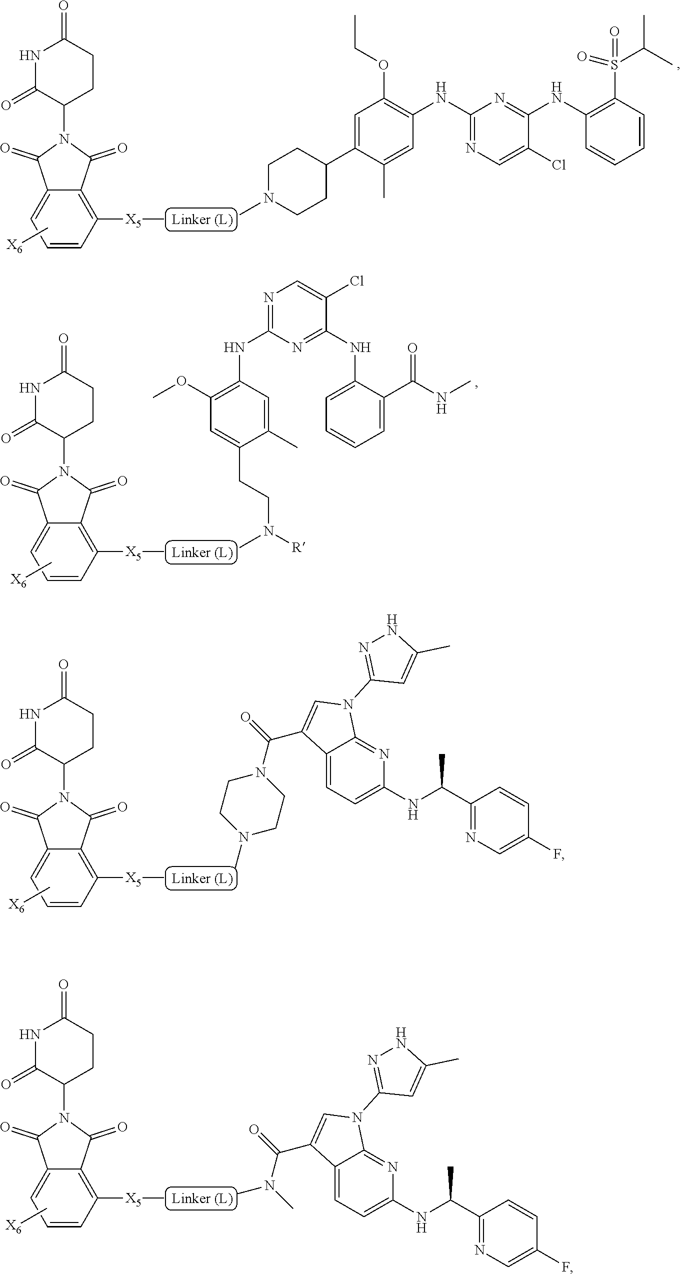

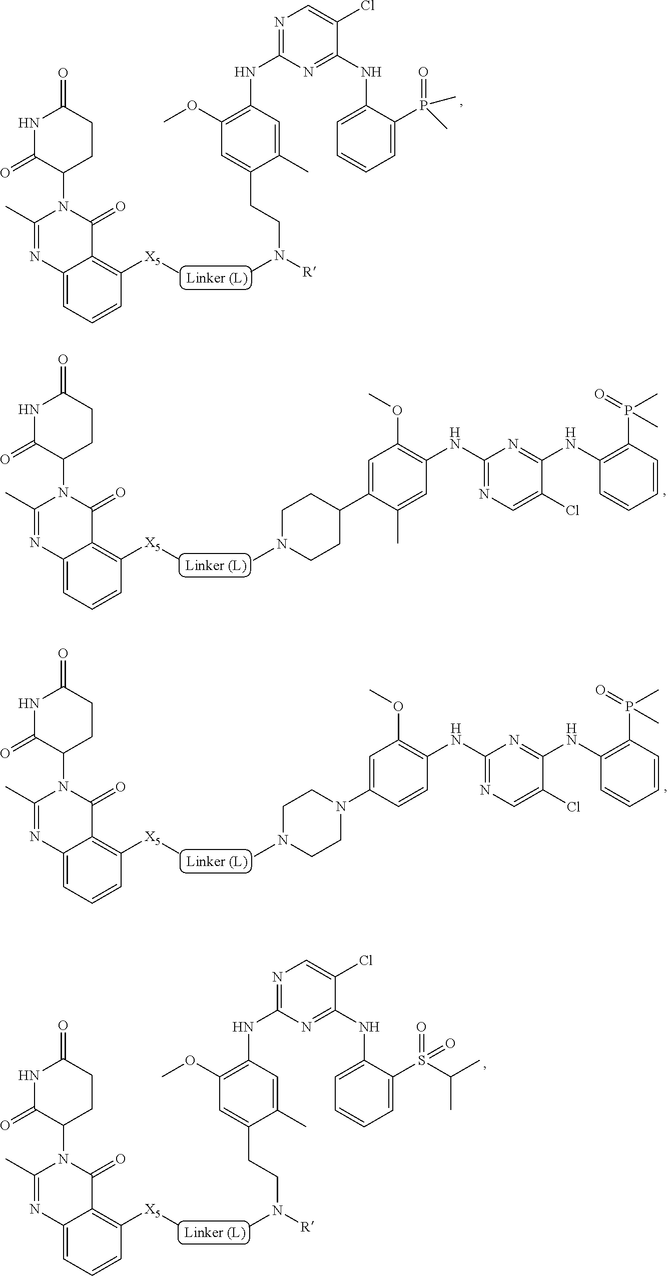

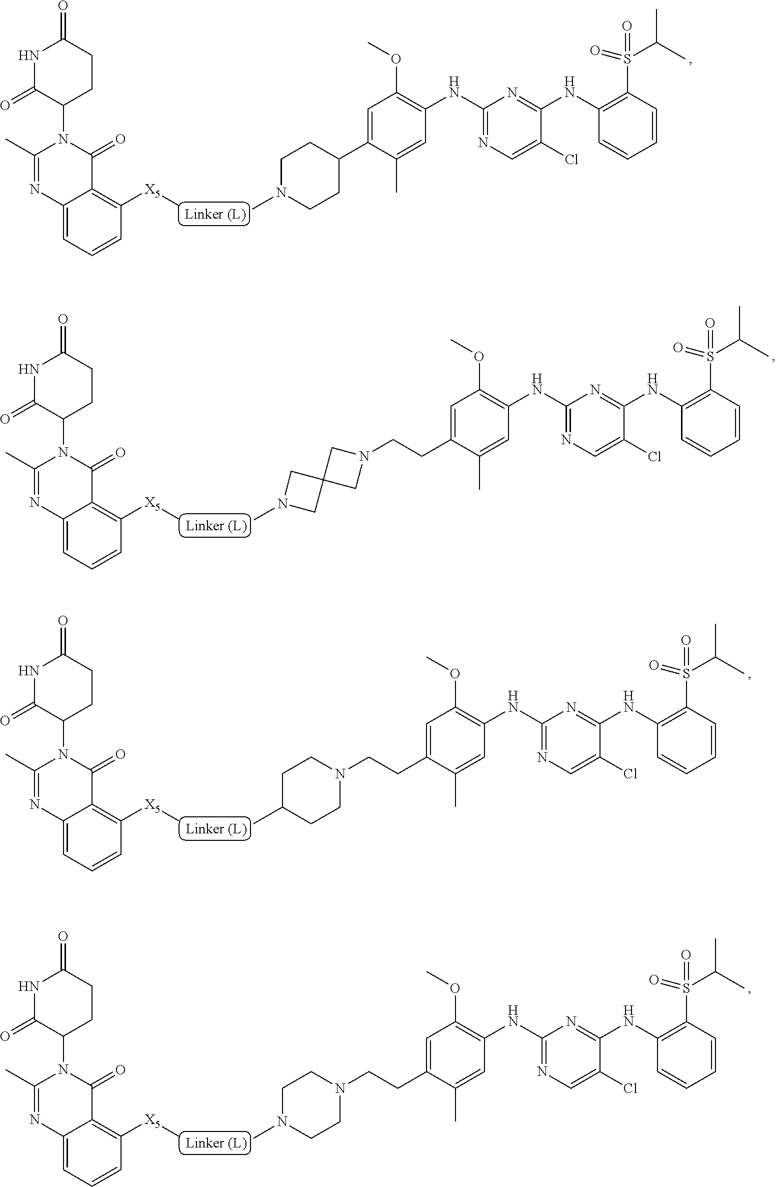

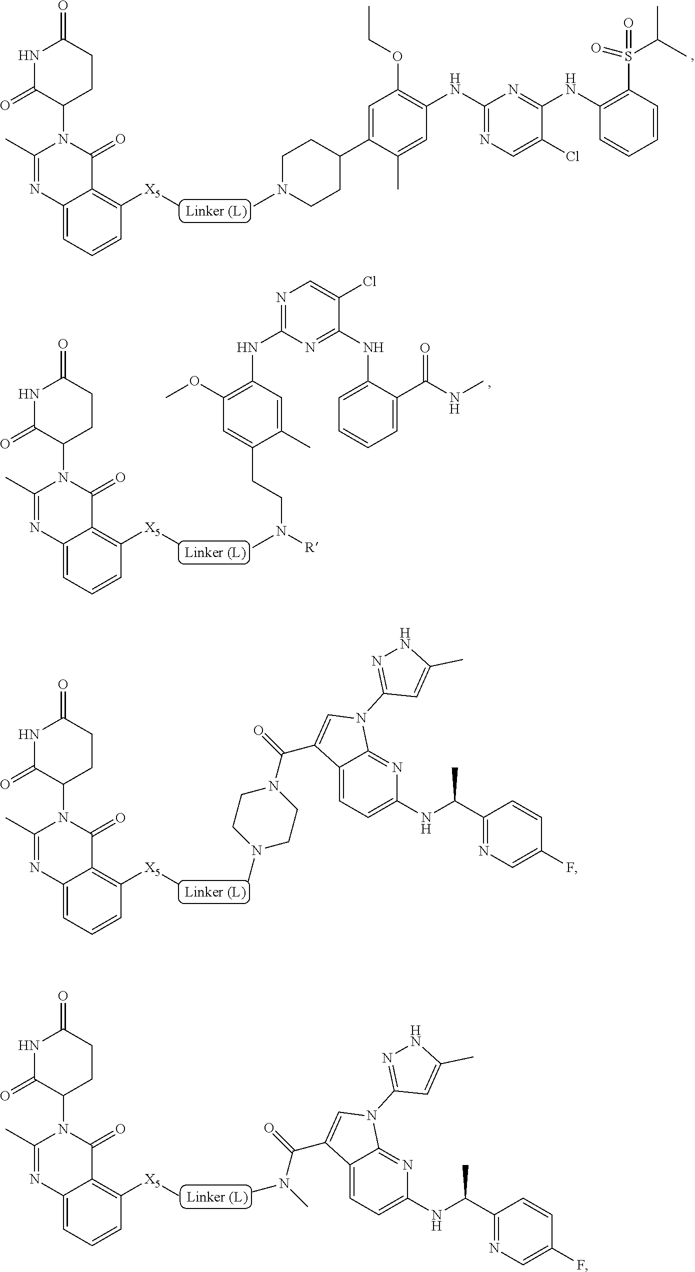

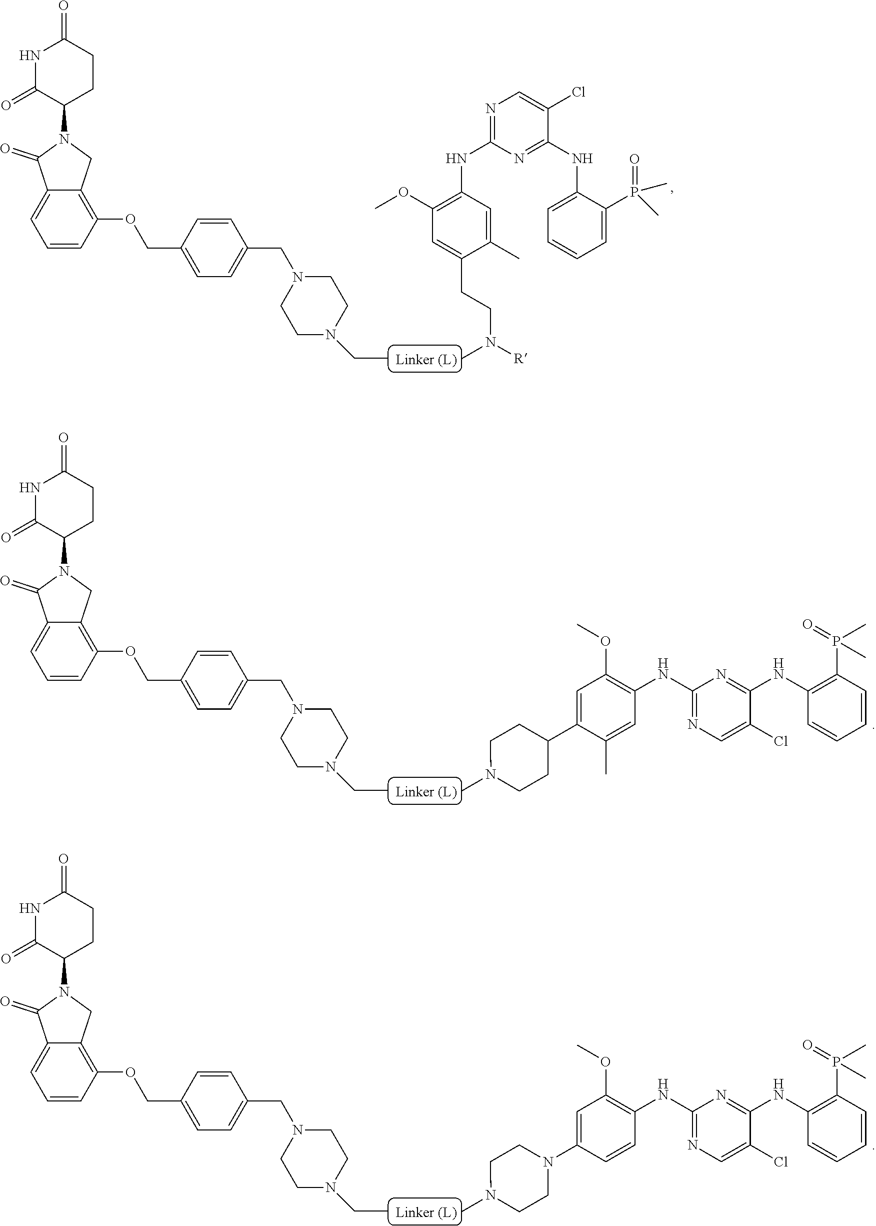

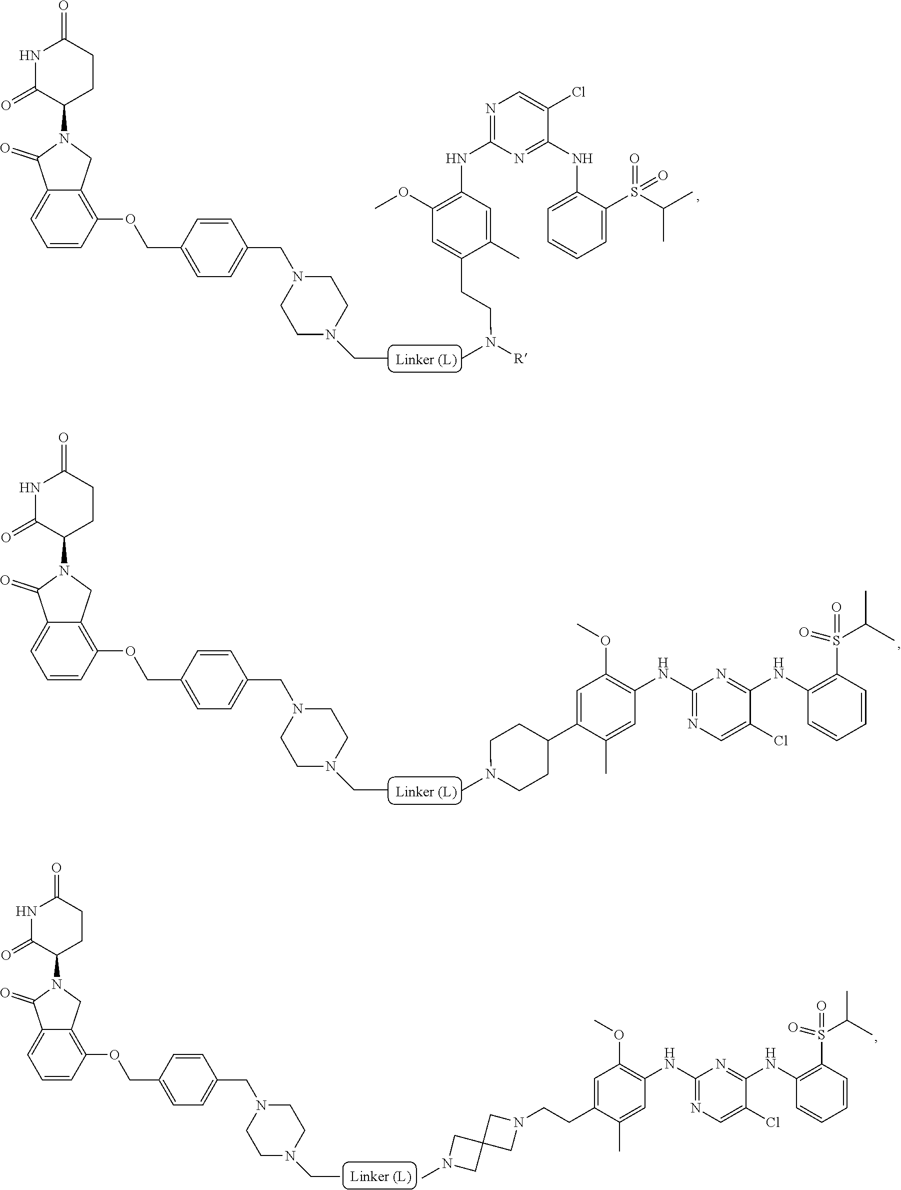

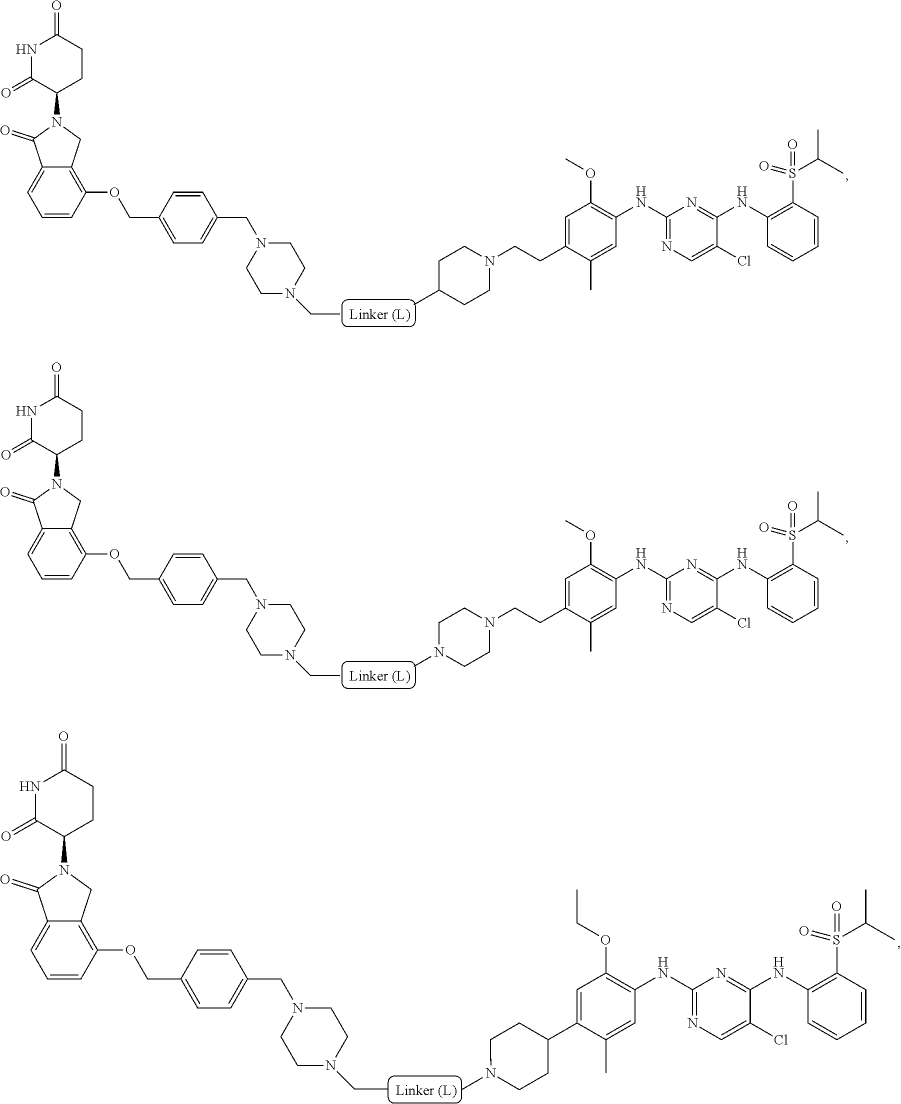 [SMILES: CCN1CCN(Cc2ccc(COc3cccc4c3CN([C@@H]3CCC(=O)NC3=O)C4=O)cc2)CC1 ; COc1cc(CCN2CCC(C)CC2)c(C)cc1Nc1ncc(Cl)c(Nc2ccccc2S(=O)(=O)C(C)C)n1 ; CCN1CCN(Cc2ccc(COc3cccc4c3CN([C@@H]3CCC(=O)NC3=O)C4=O)cc2)CC1 ; COc1cc(CCN2CCN(C)CC2)c(C)cc1Nc1ncc(Cl)c(Nc2ccccc2S(=O)(=O)C(C)C)n1 ; CCN1CCN(Cc2ccc(COc3cccc4c3CN([C@@H]3CCC(=O)NC3=O)C4=O)cc2)CC1 ; CCOc1cc(C2CCN(C)CC2)c(C)cc1Nc1ncc(Cl)c(Nc2ccccc2S(=O)(=O)C(C)C)n1]

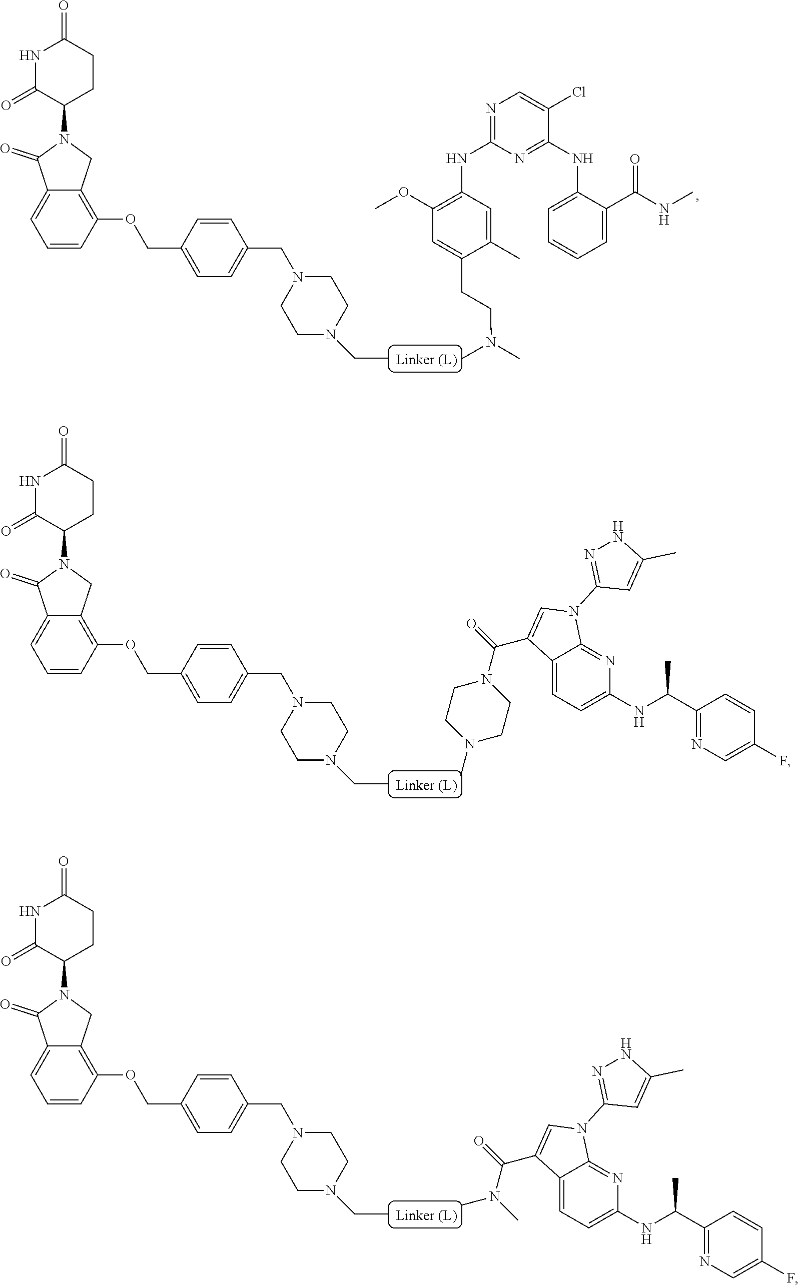

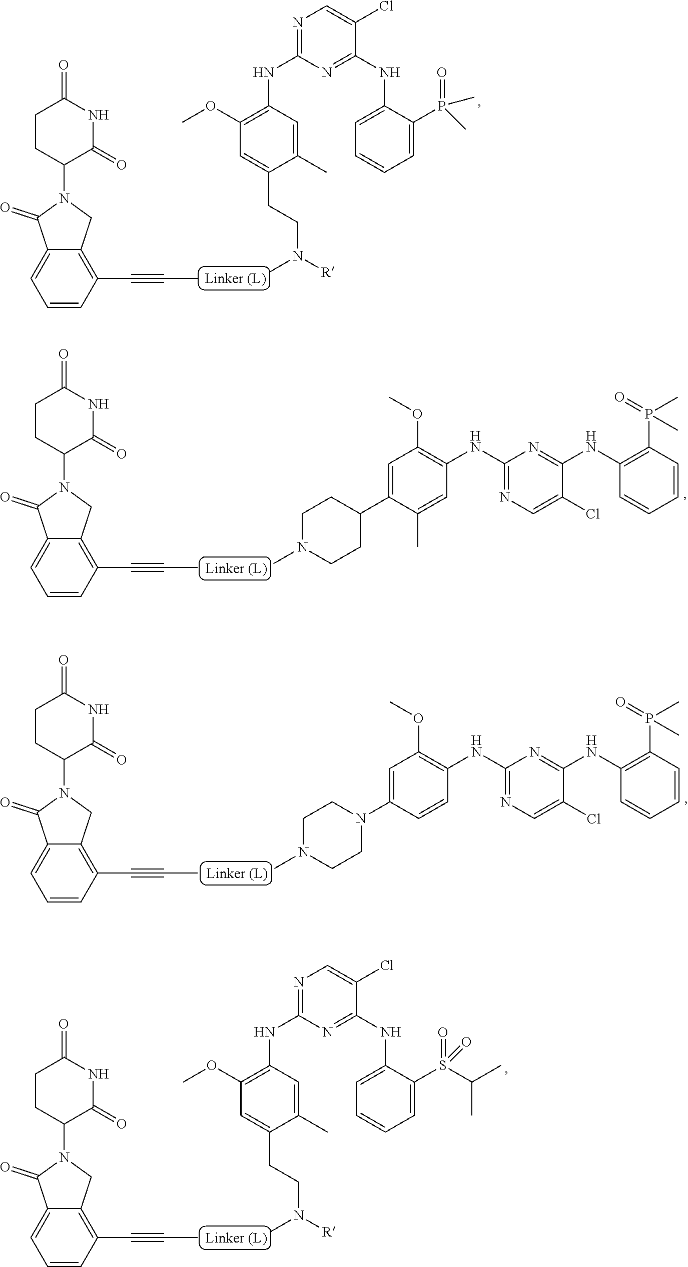

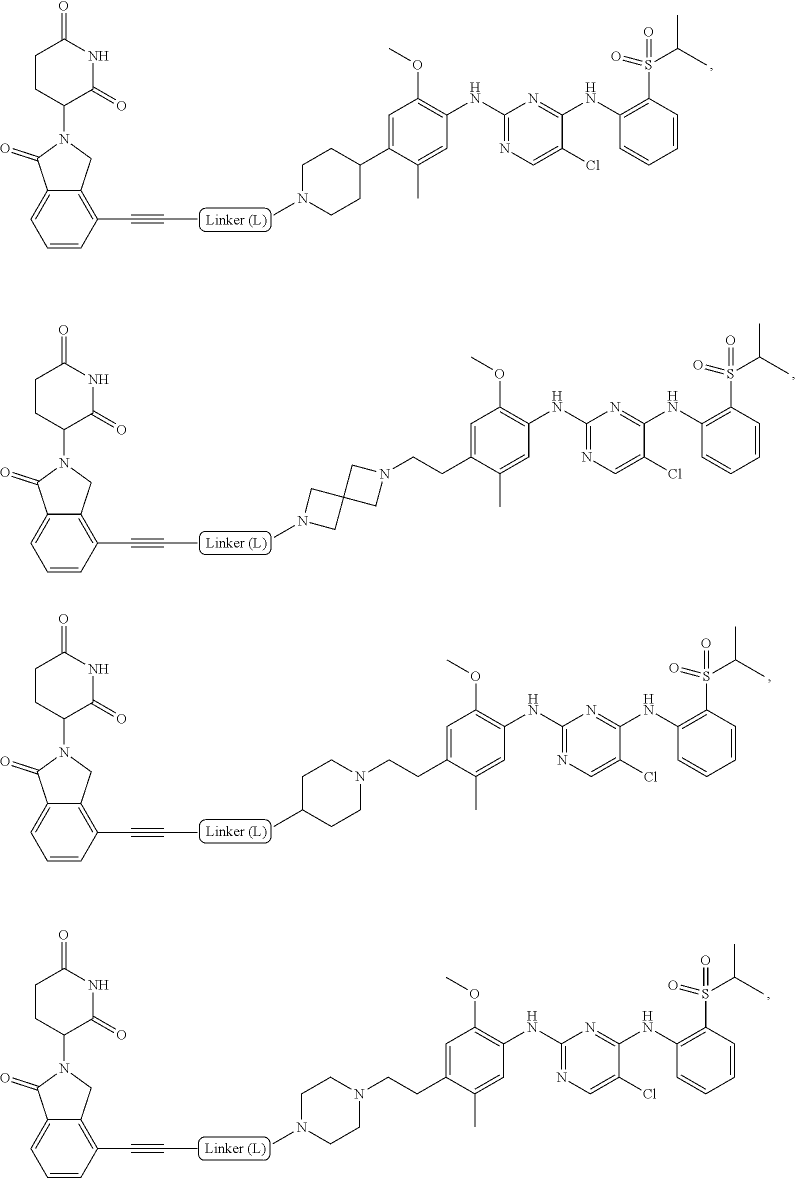

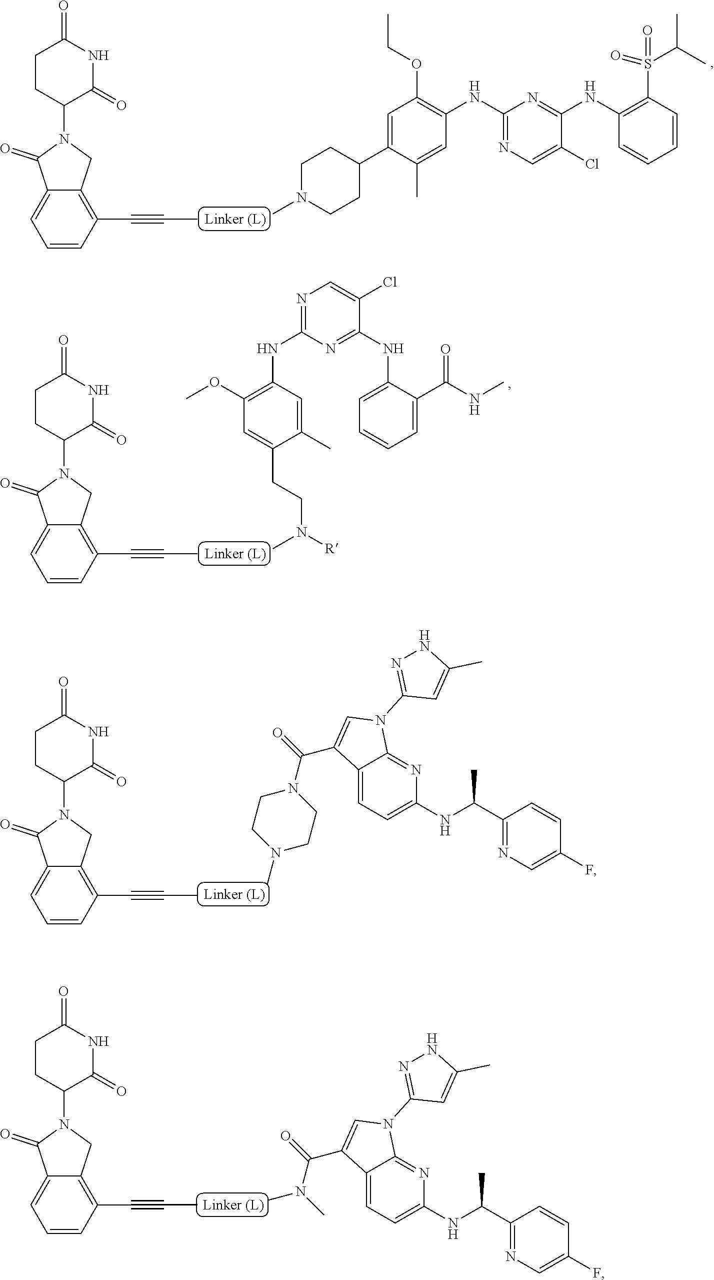

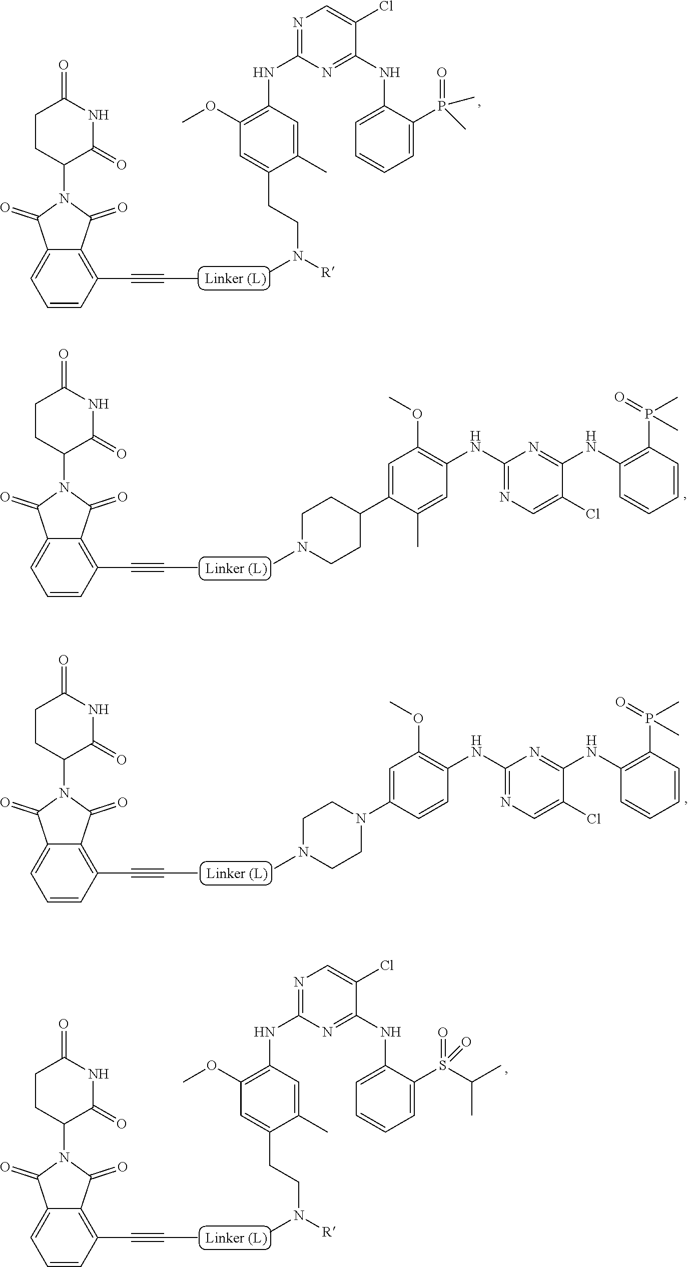

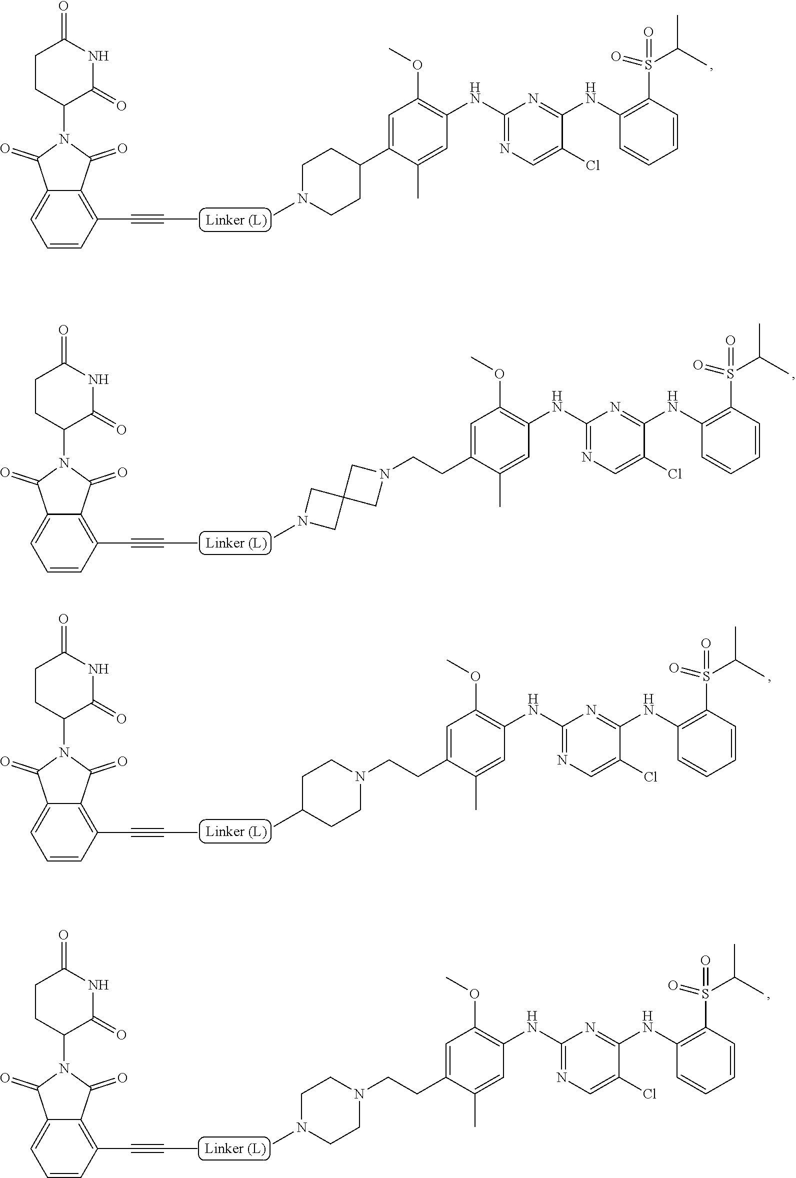

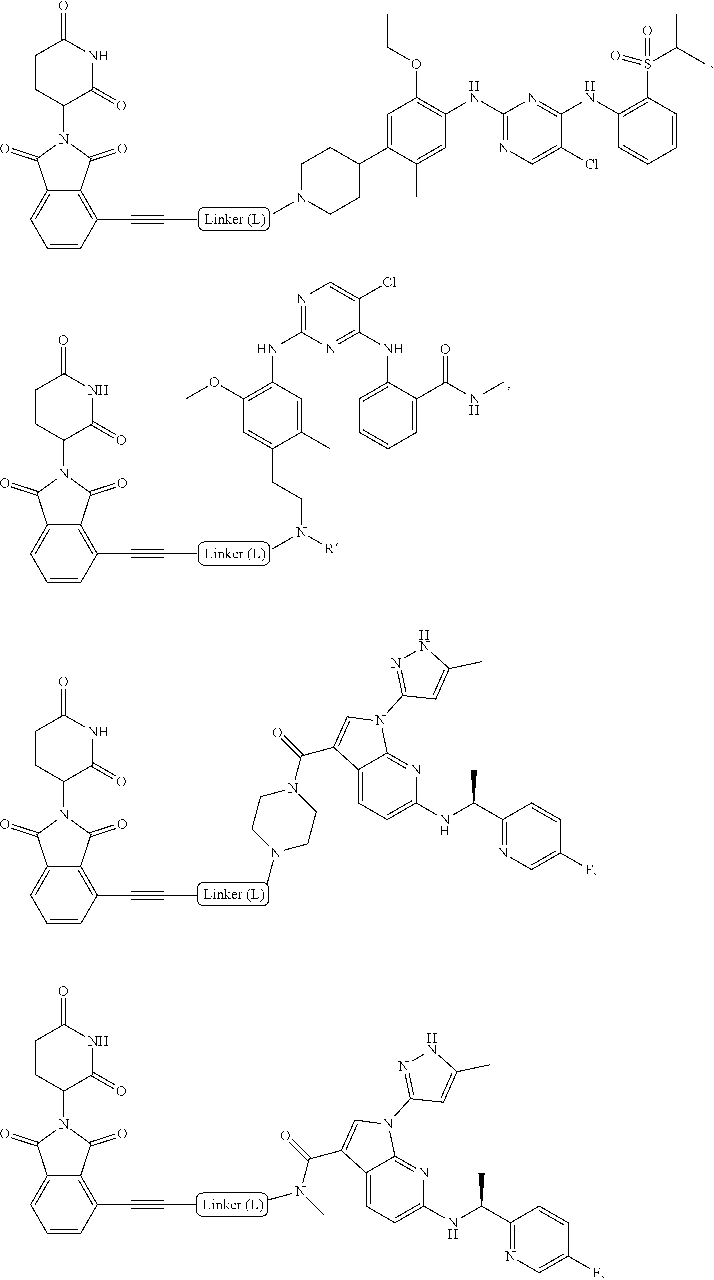

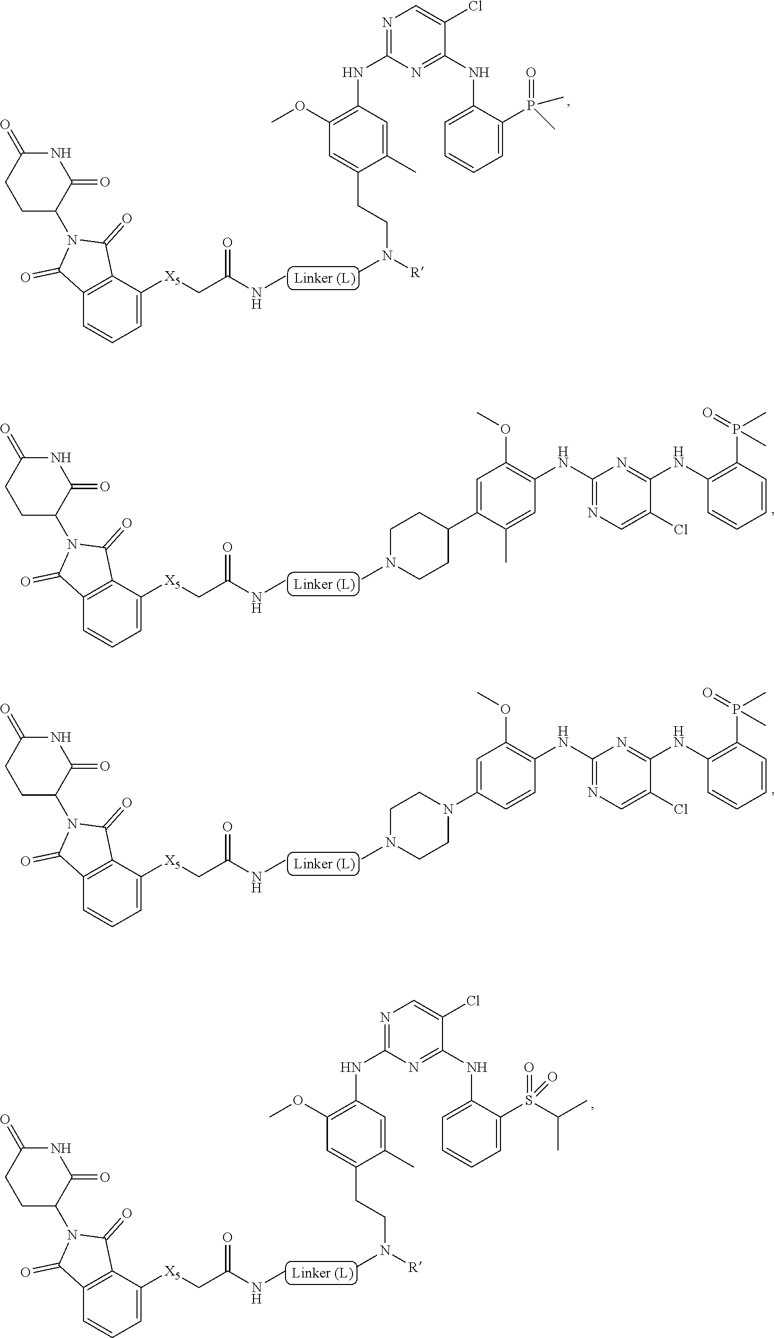

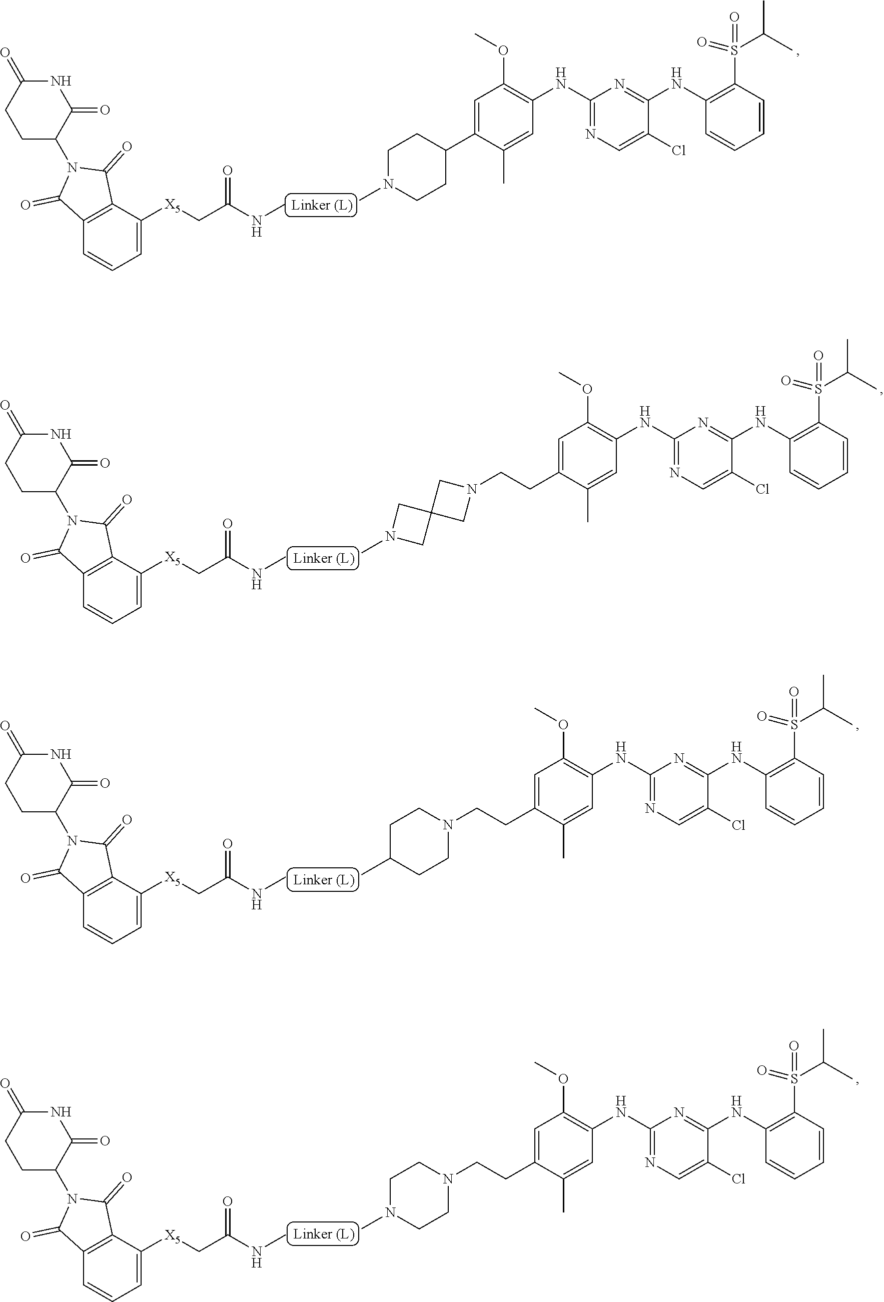

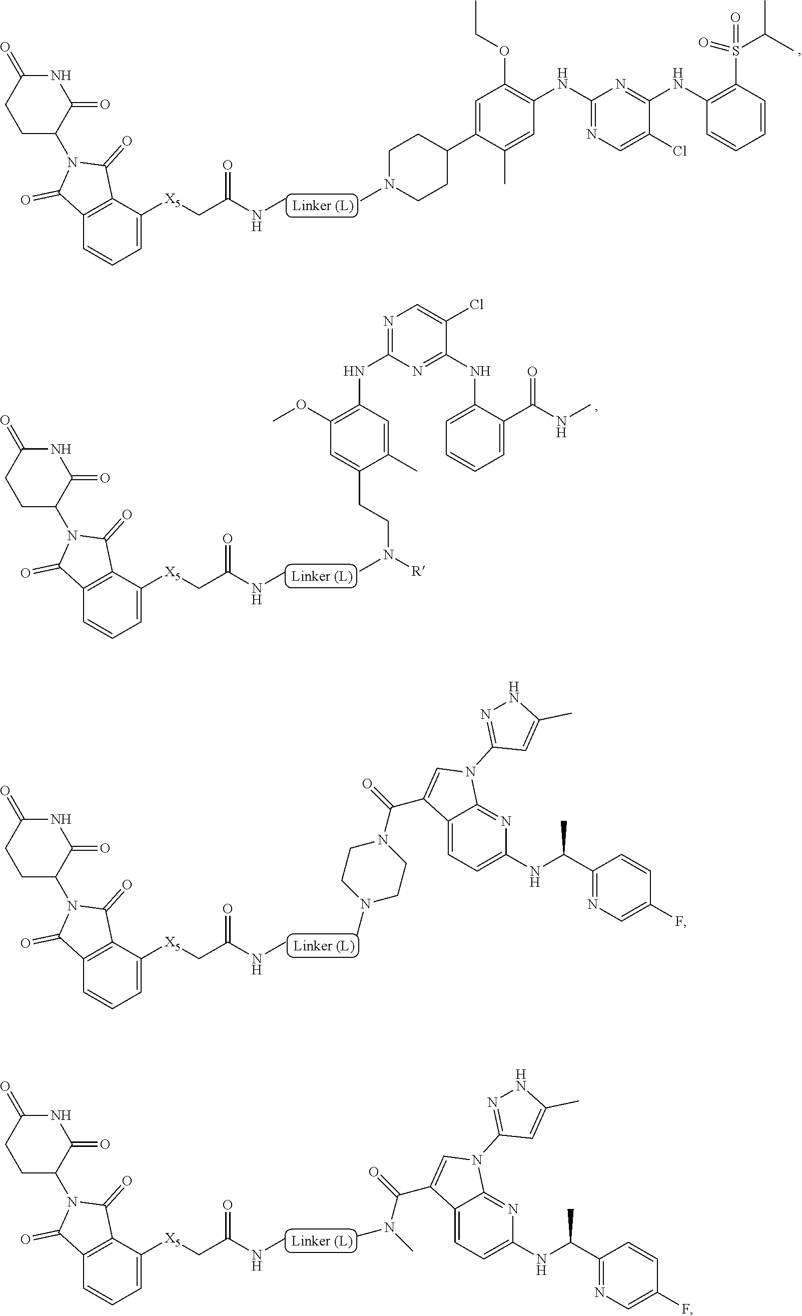

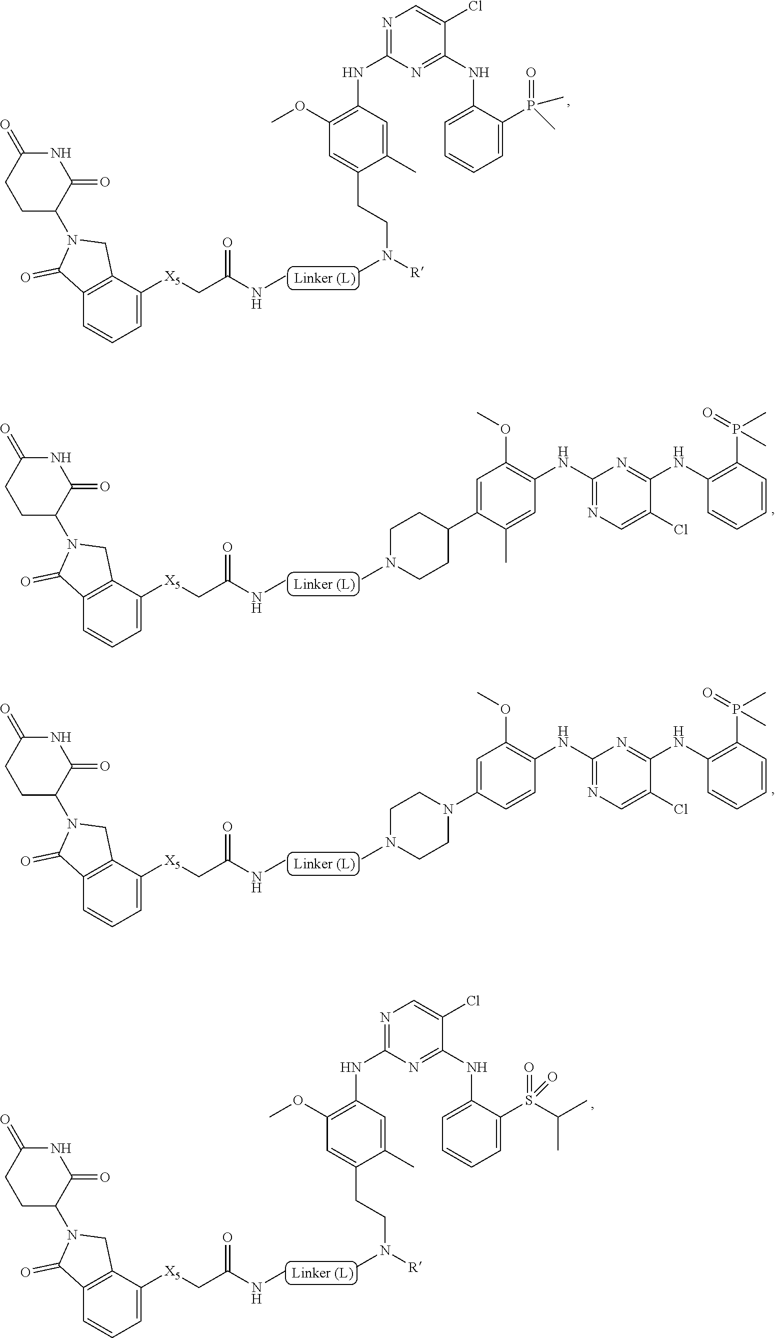

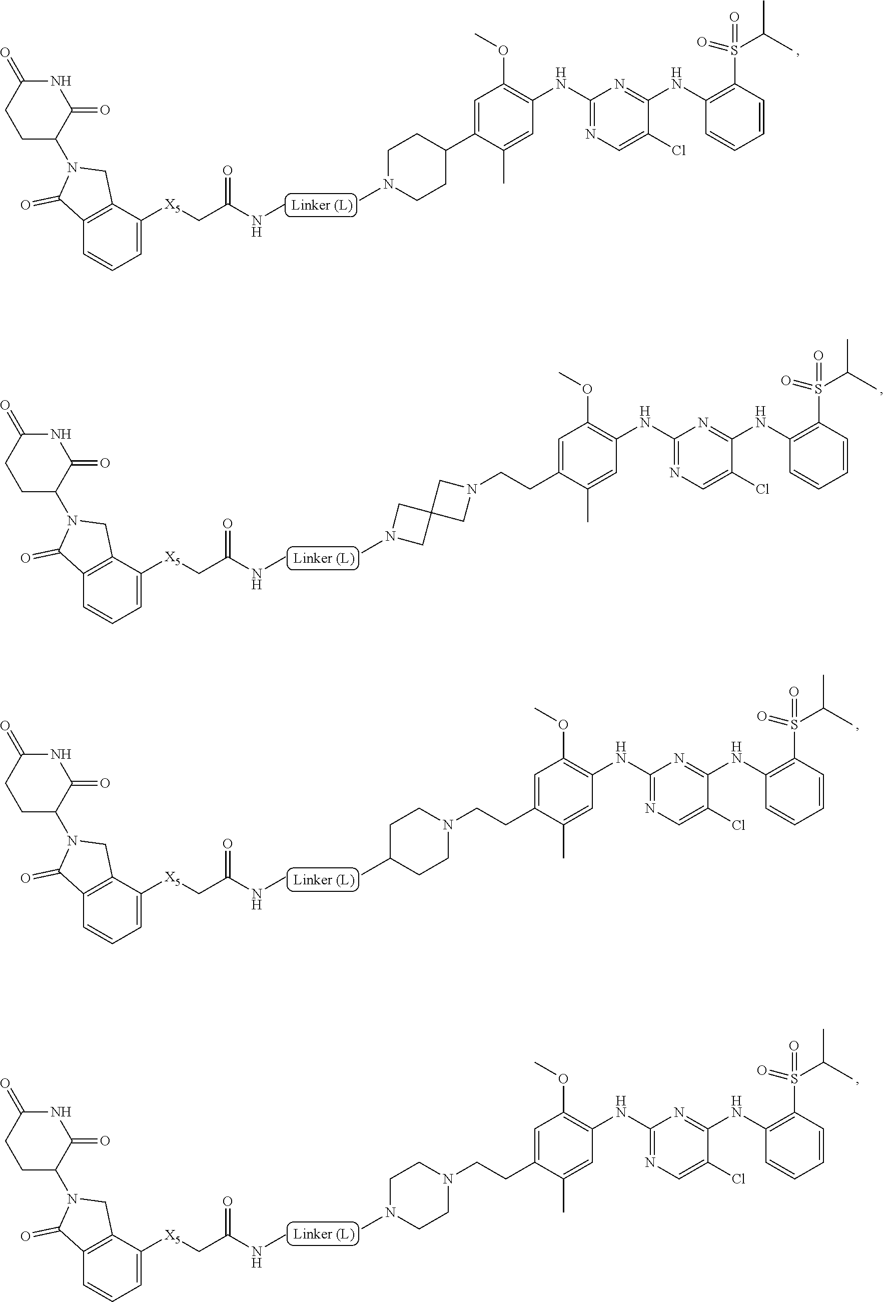

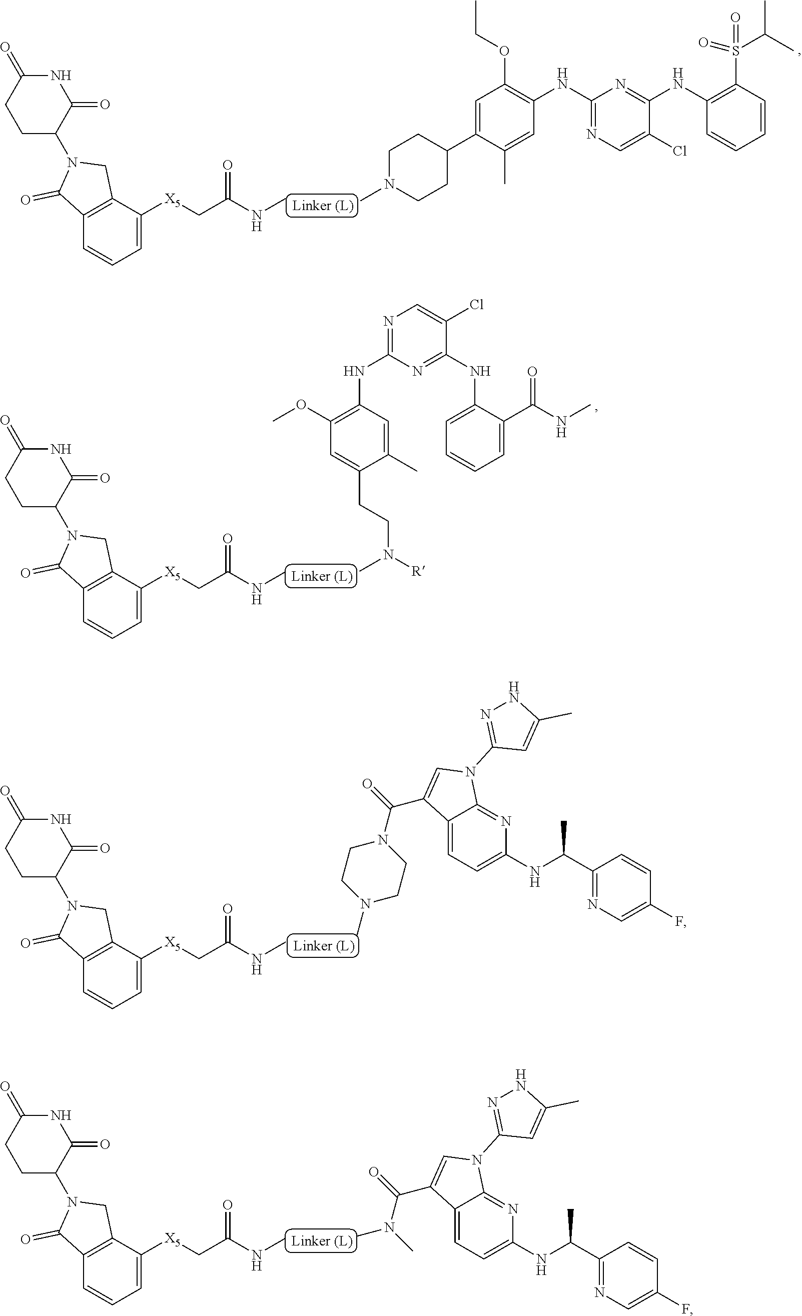

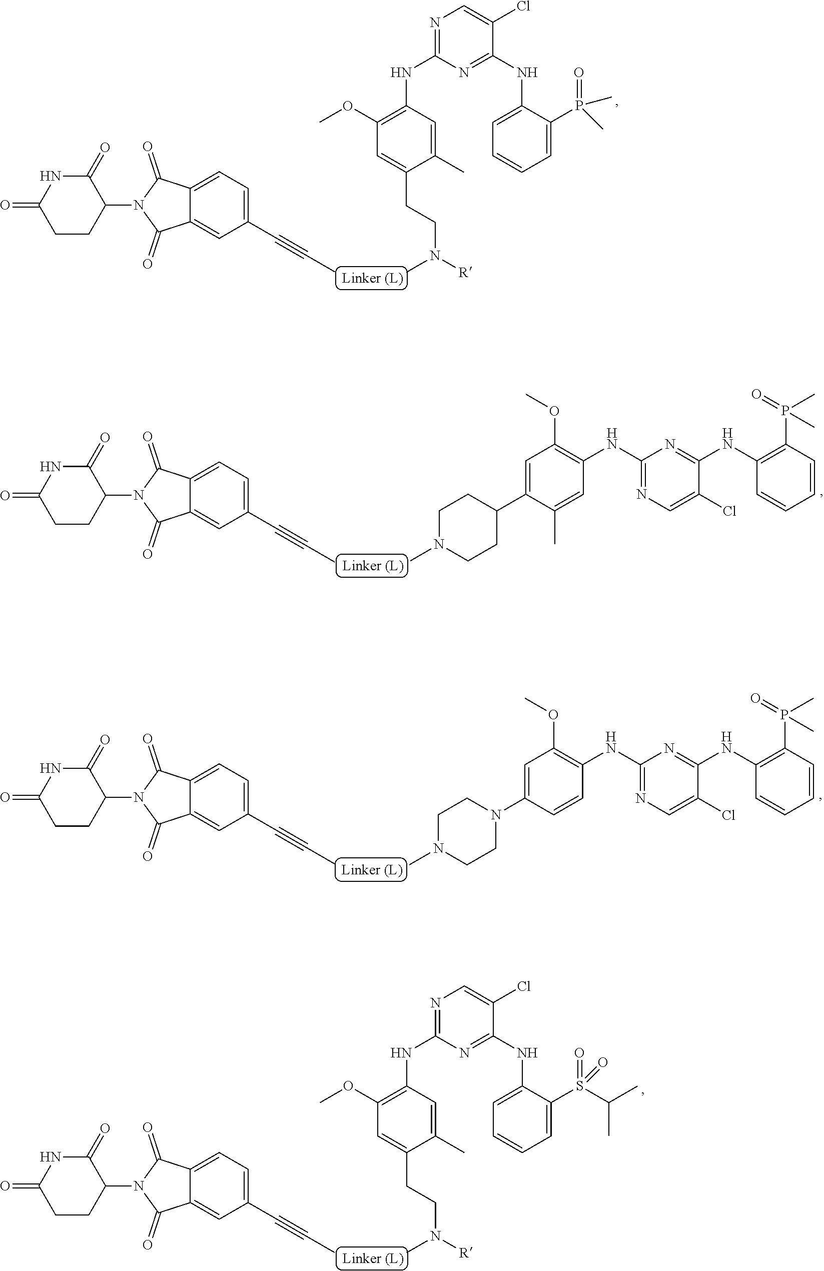

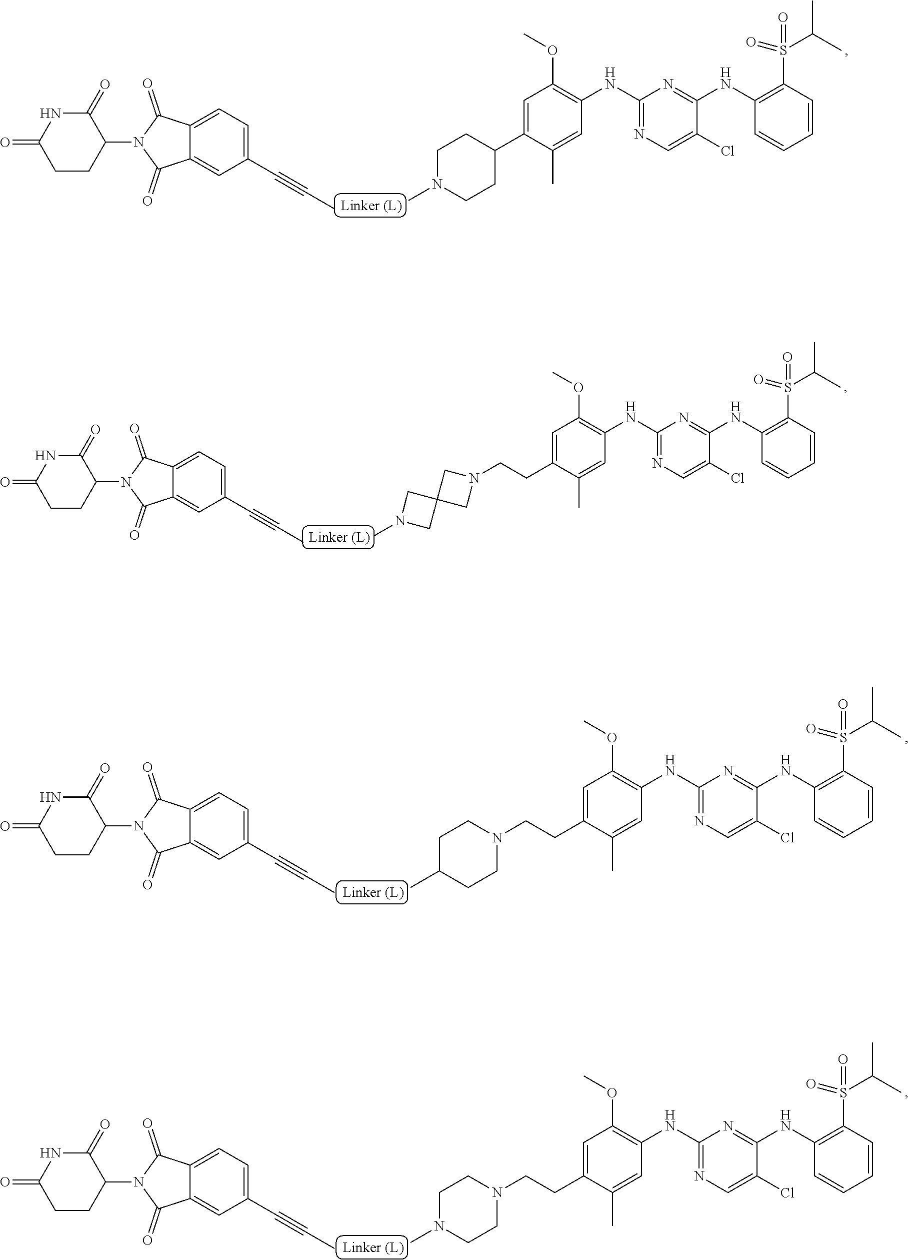

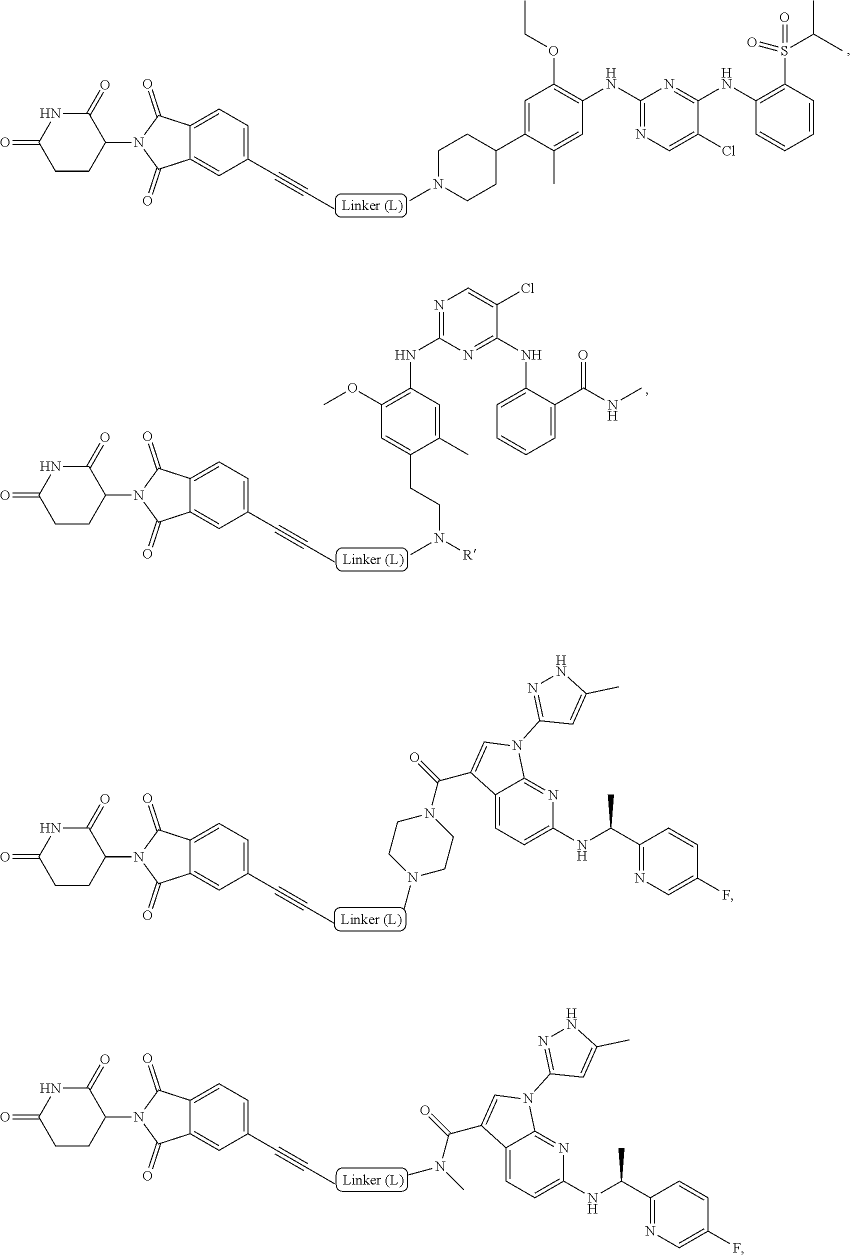

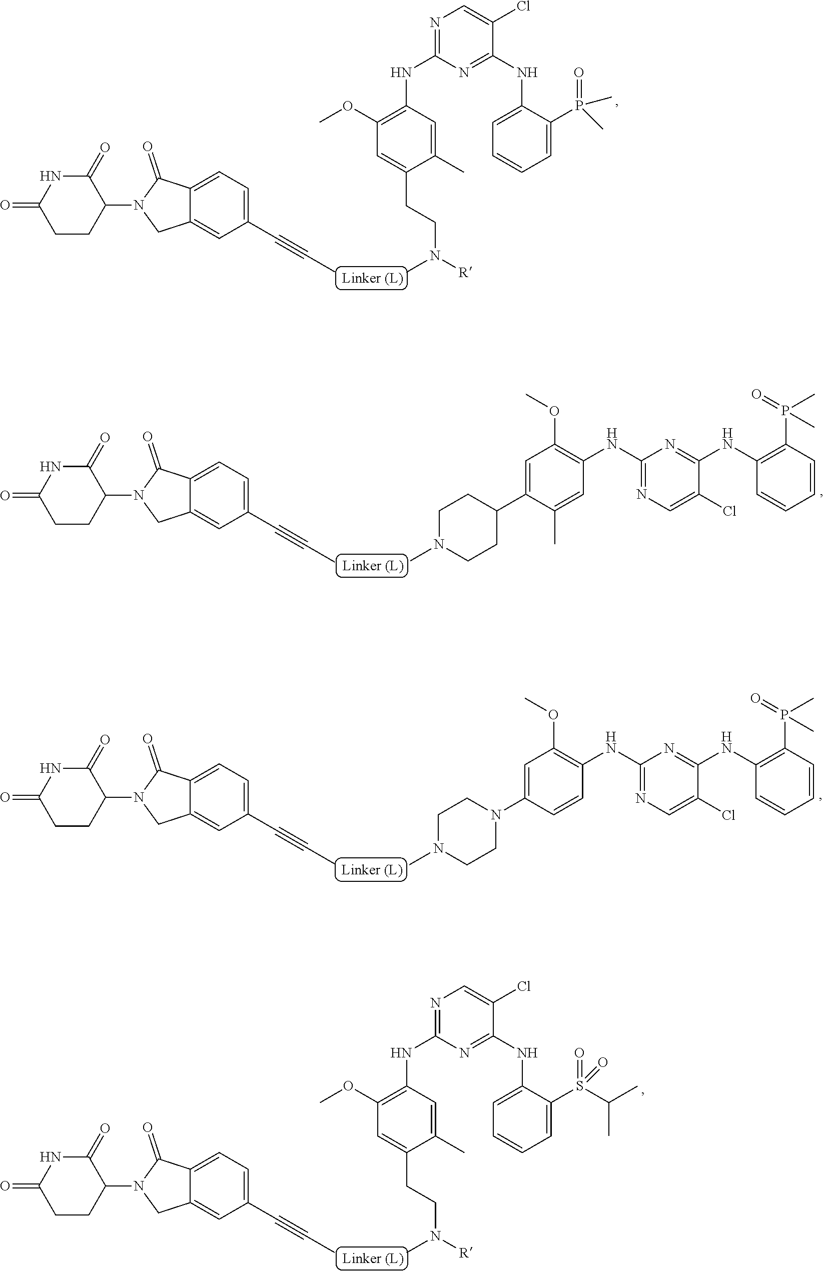

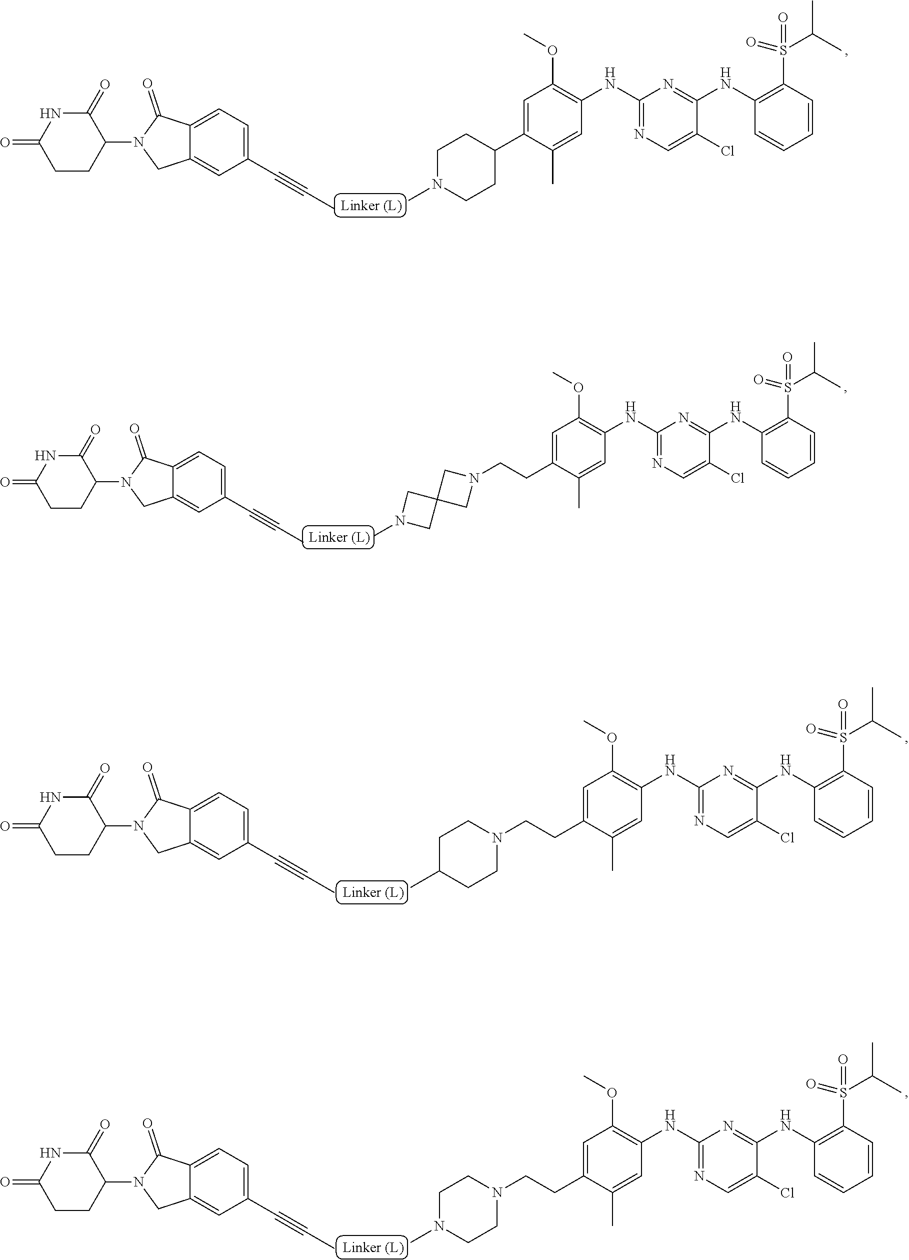

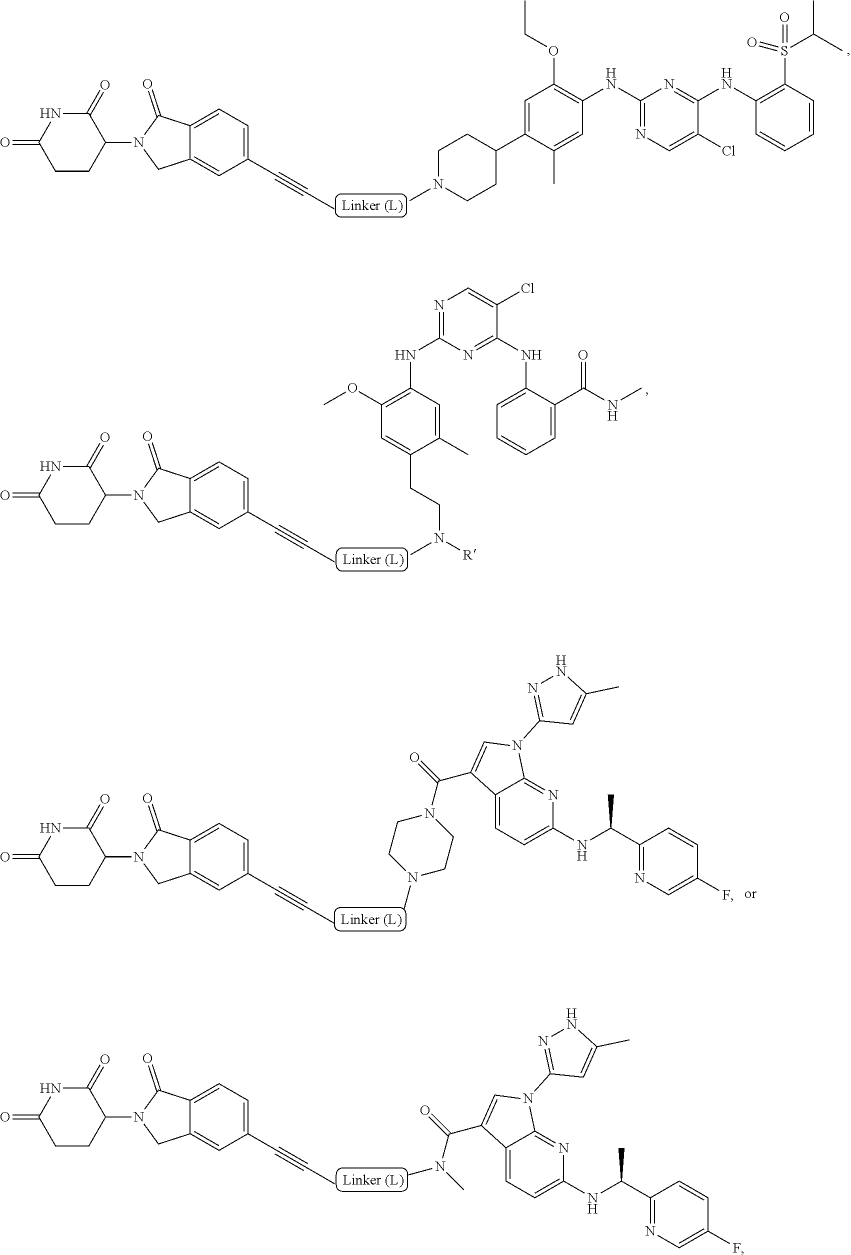

or a pharmaceutically acceptable salt or stereoisomer thereof.

Yet other degrons that bind cereblon and which may be suitable for use in the present invention are disclosed in U.S. Patent Application Publication 2018/0015085 A1 (e.g., the indolinones such as isoindolinones and isoindoline-1,3-diones embraced by formulae IA ad IA′ therein, and the bridged cycloalkyl compounds embraced by formulae IB and IB′ therein).

In some embodiments, the E3 ubiquitin ligase that is bound by the degron is the von Hippel-Lindau (VHL) tumor suppressor. See, Iwai et al., Proc. Nat'l. Acad. Sci. USA 96:12436-41 (1999).

Representative examples of degrons that bind VHL are as follows:

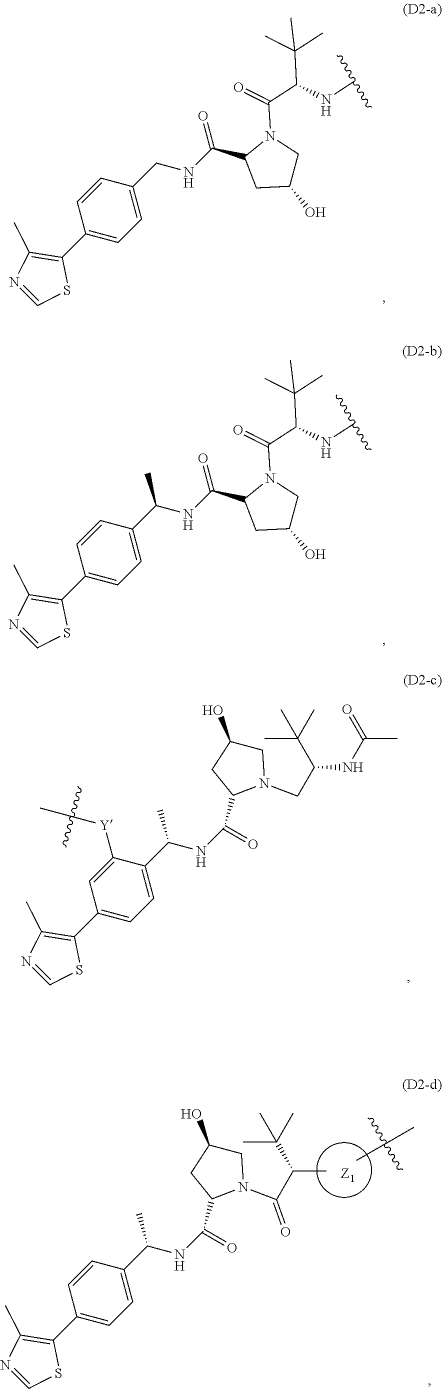

wherein Z₁ is a cyclic group,

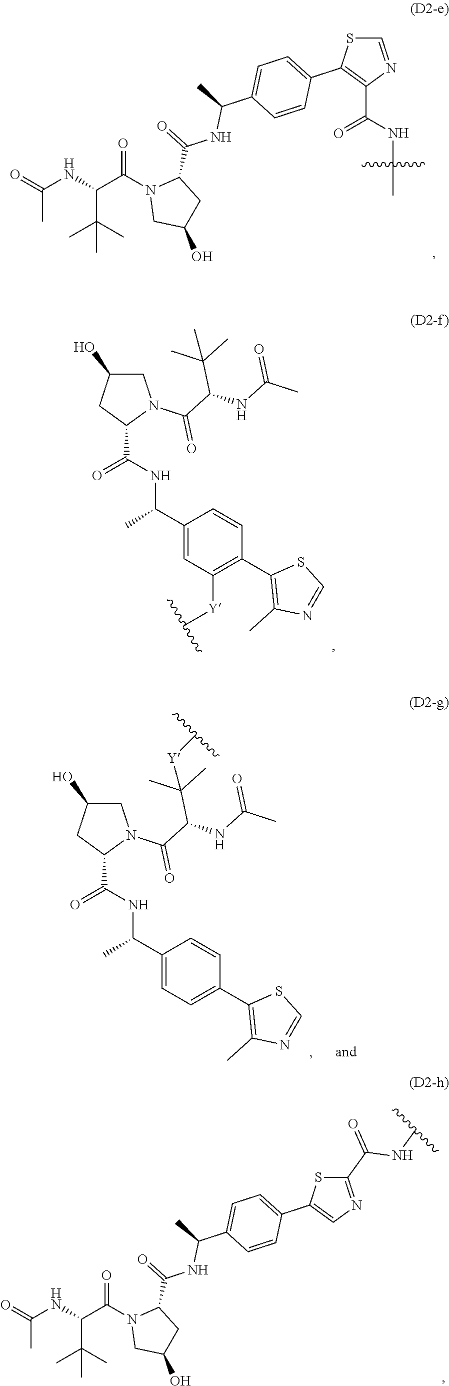

wherein Y′ is a bond, CH₂, NH, NMe, O, or S, or a stereoisomer thereof.

In certain embodiments, Z₁ is a 5-6 membered cyclic or a 5-6 membered heterocyclic group. In some embodiments, Z₁ is

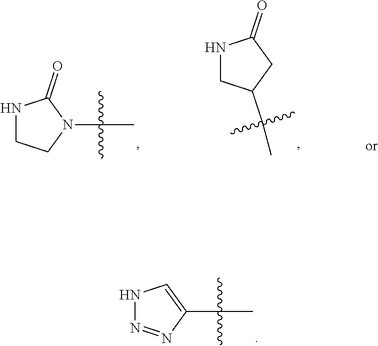 [SMILES: *N1CCNC1=O ; *C1CNC(=O)C1 ; *c1c[nH]nn1]

Therefore, in some embodiments, bispecific compounds of the present invention are represented by any one of the following structures:

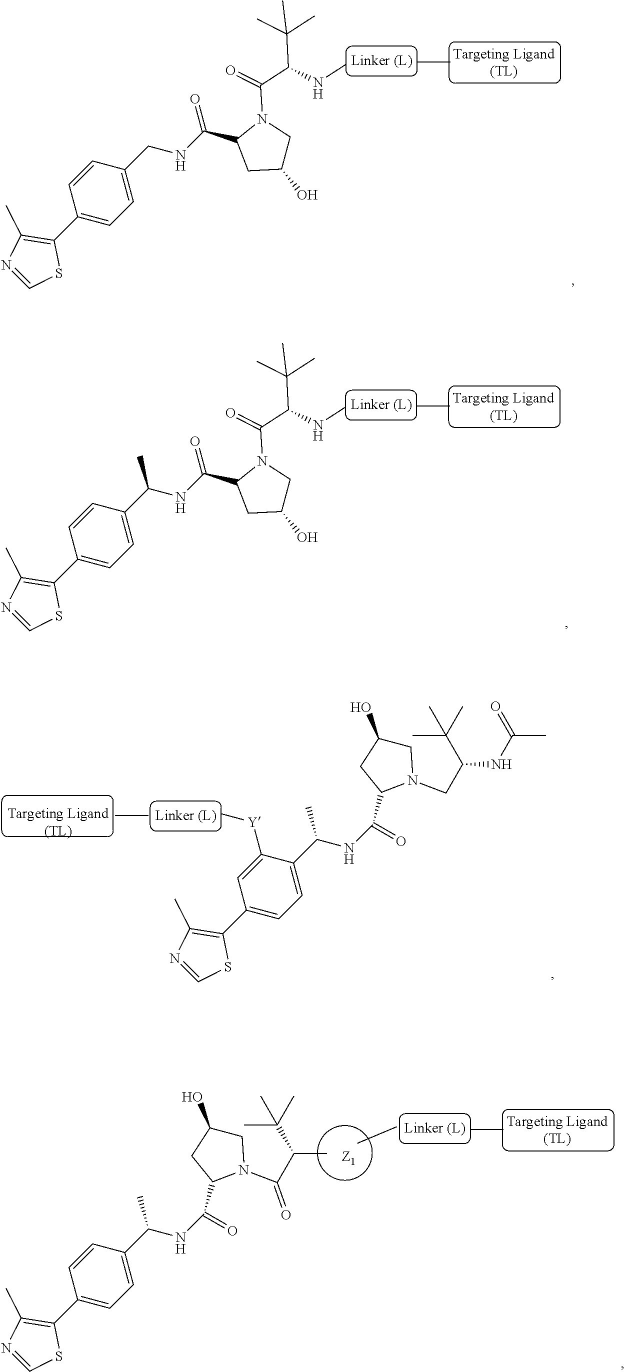

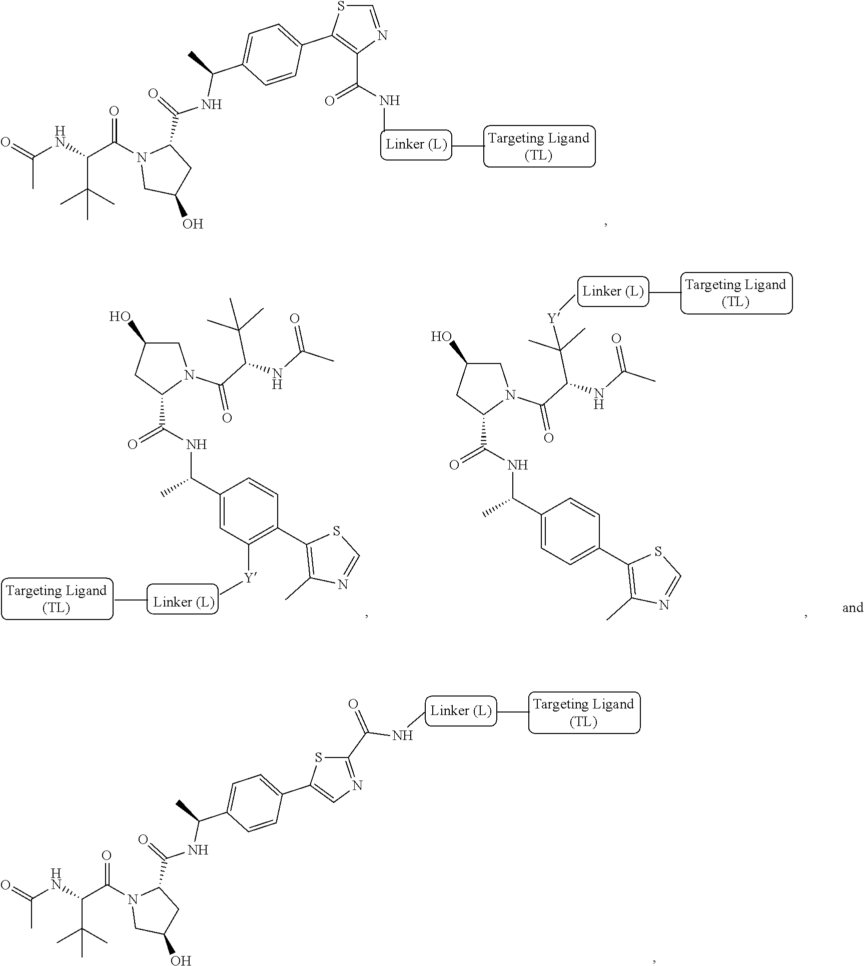

or a pharmaceutically acceptable salt or stereoisomer thereof.

In some embodiments, Z₁ is phenyl, pyrrolyl, furanyl, thiophenyl, pyrazolyl, imidazolyl, oxazolyl, thiazolyl, pyridinyl, pyridazinyl, or pyrimidinyl. In certain embodiments, Z is

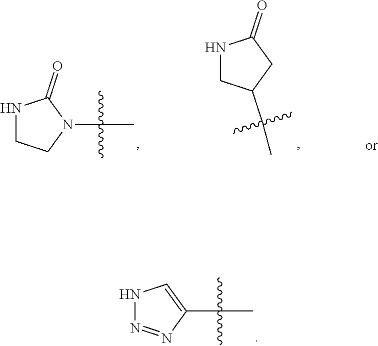 [SMILES: *N1CCNC1=O ; *C1CNC(=O)C1 ; *c1c[nH]nn1]

In some embodiments, bispecific compounds of the present invention are represented by any one of the following structures:

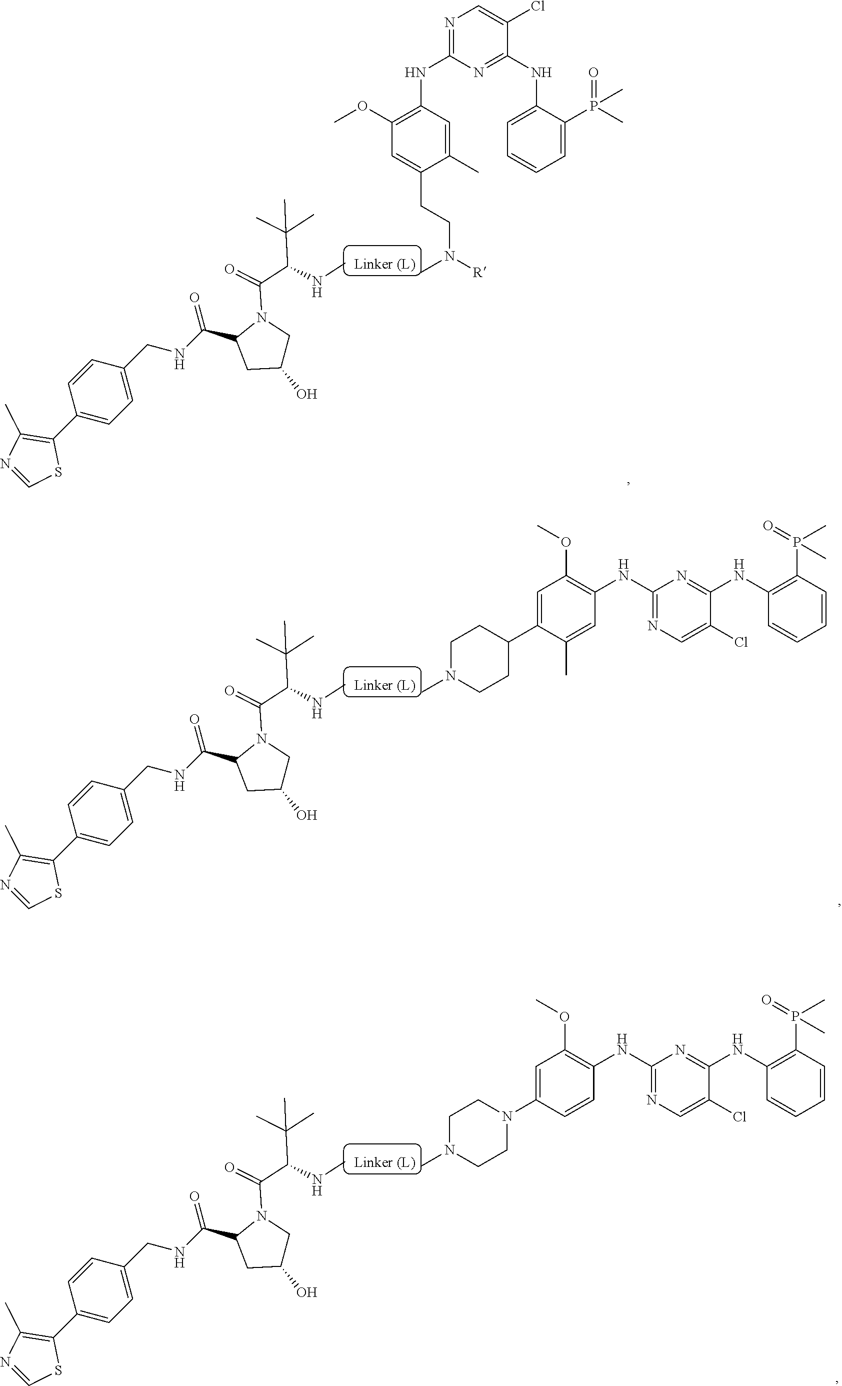

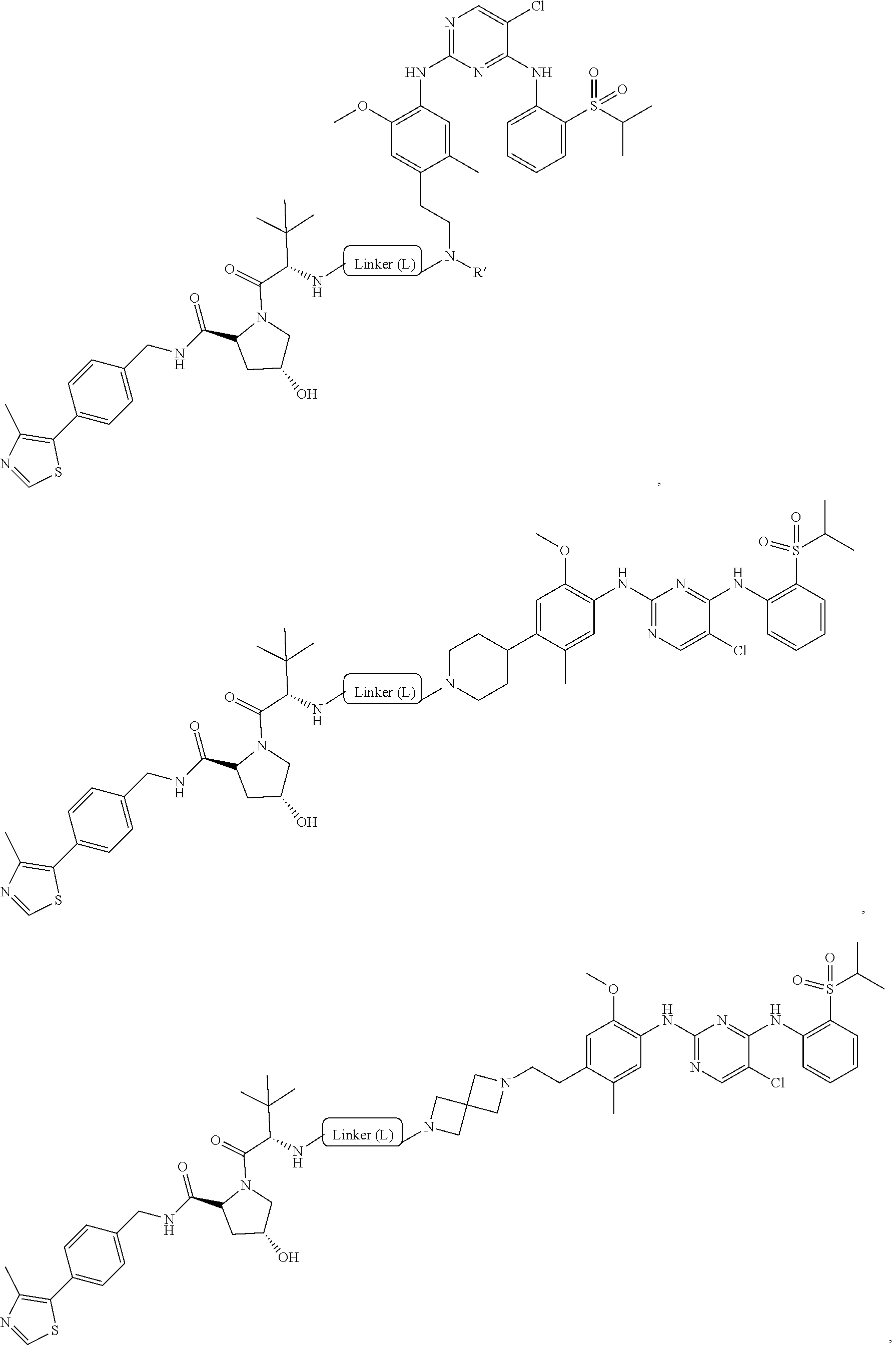

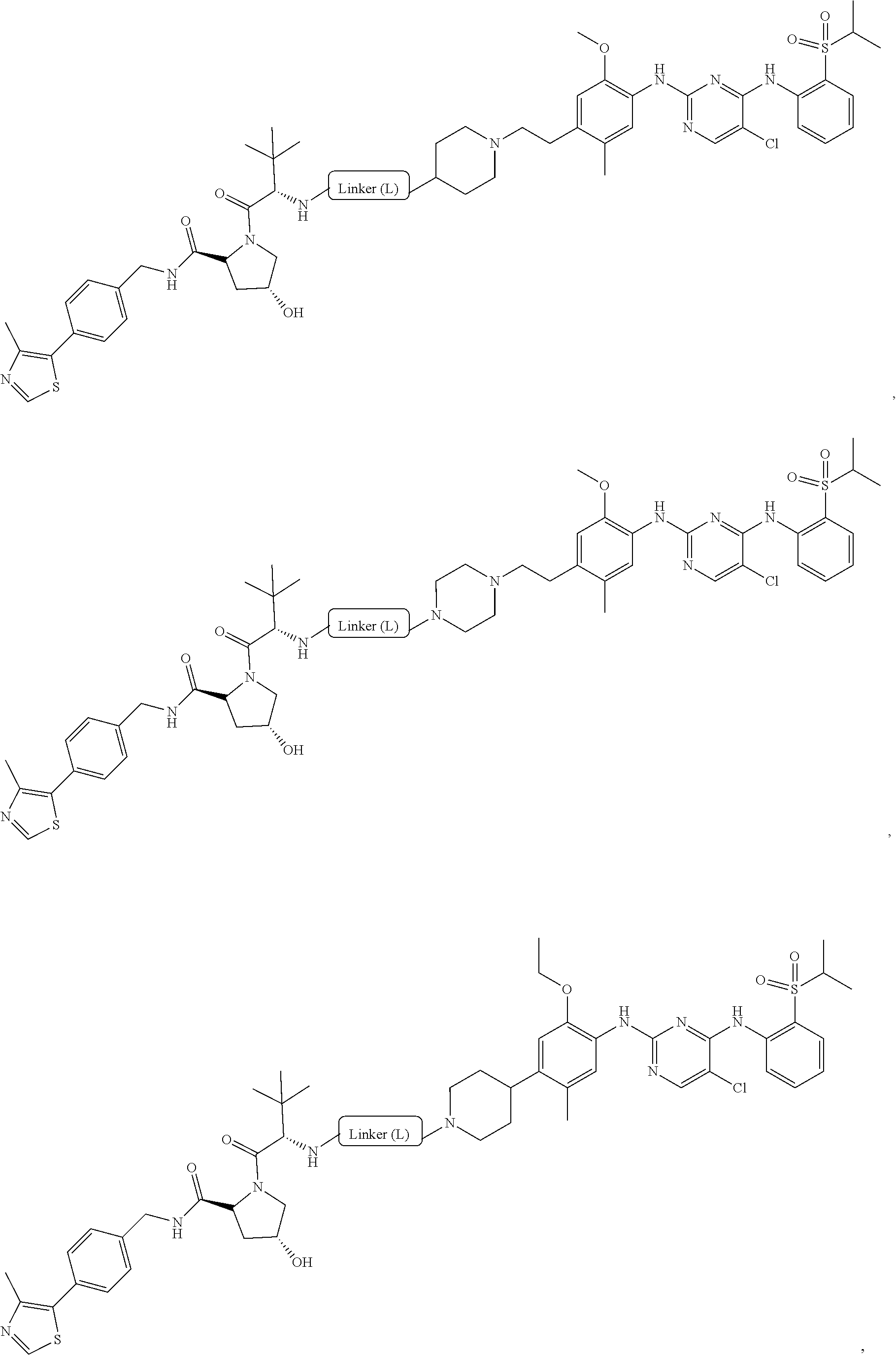

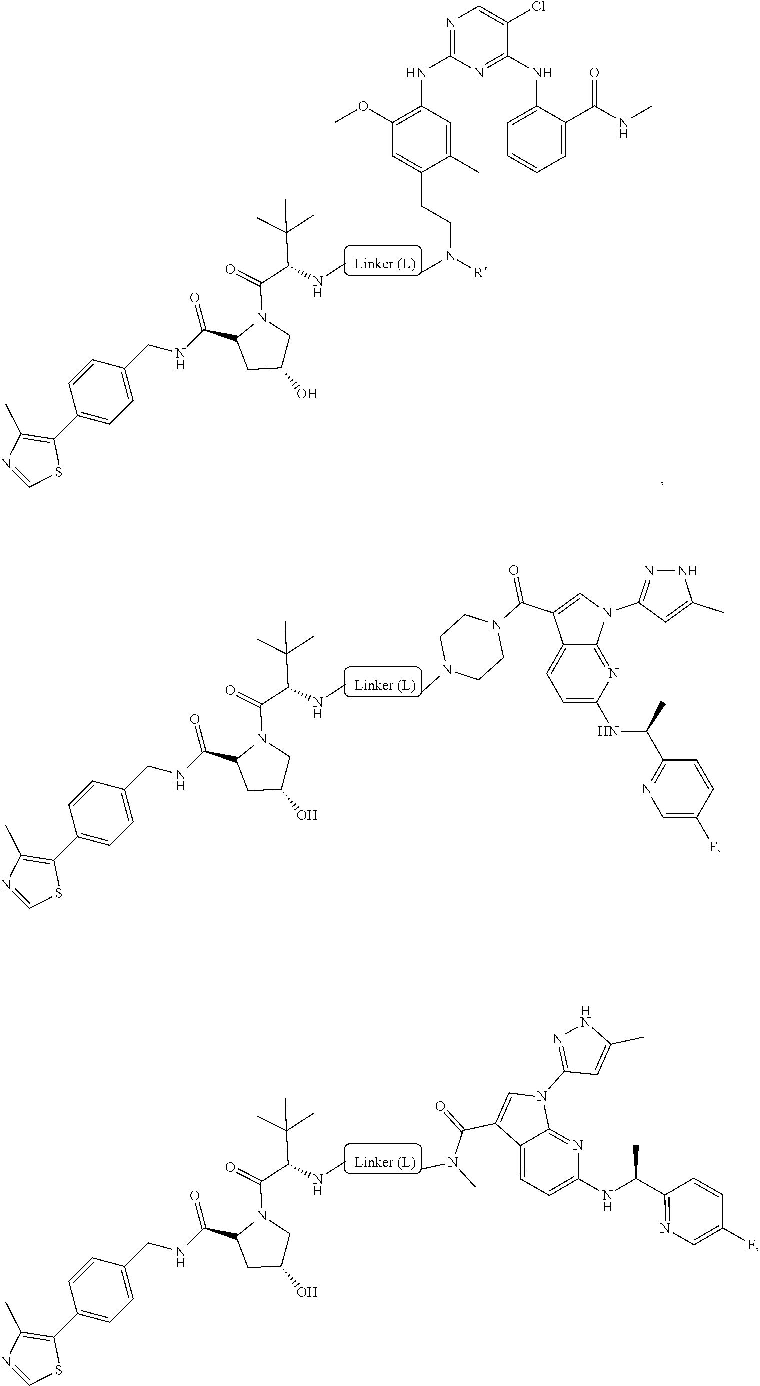

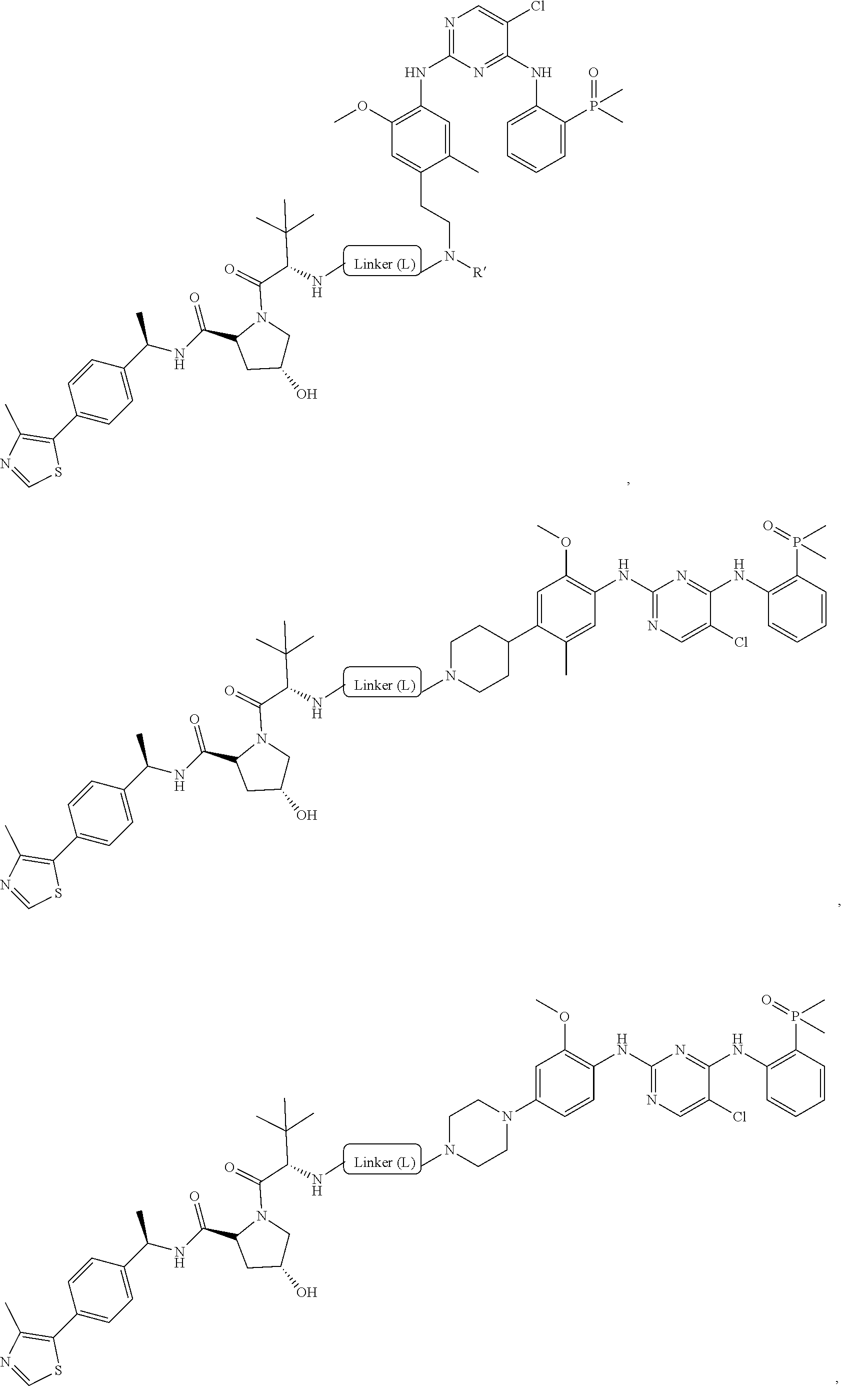

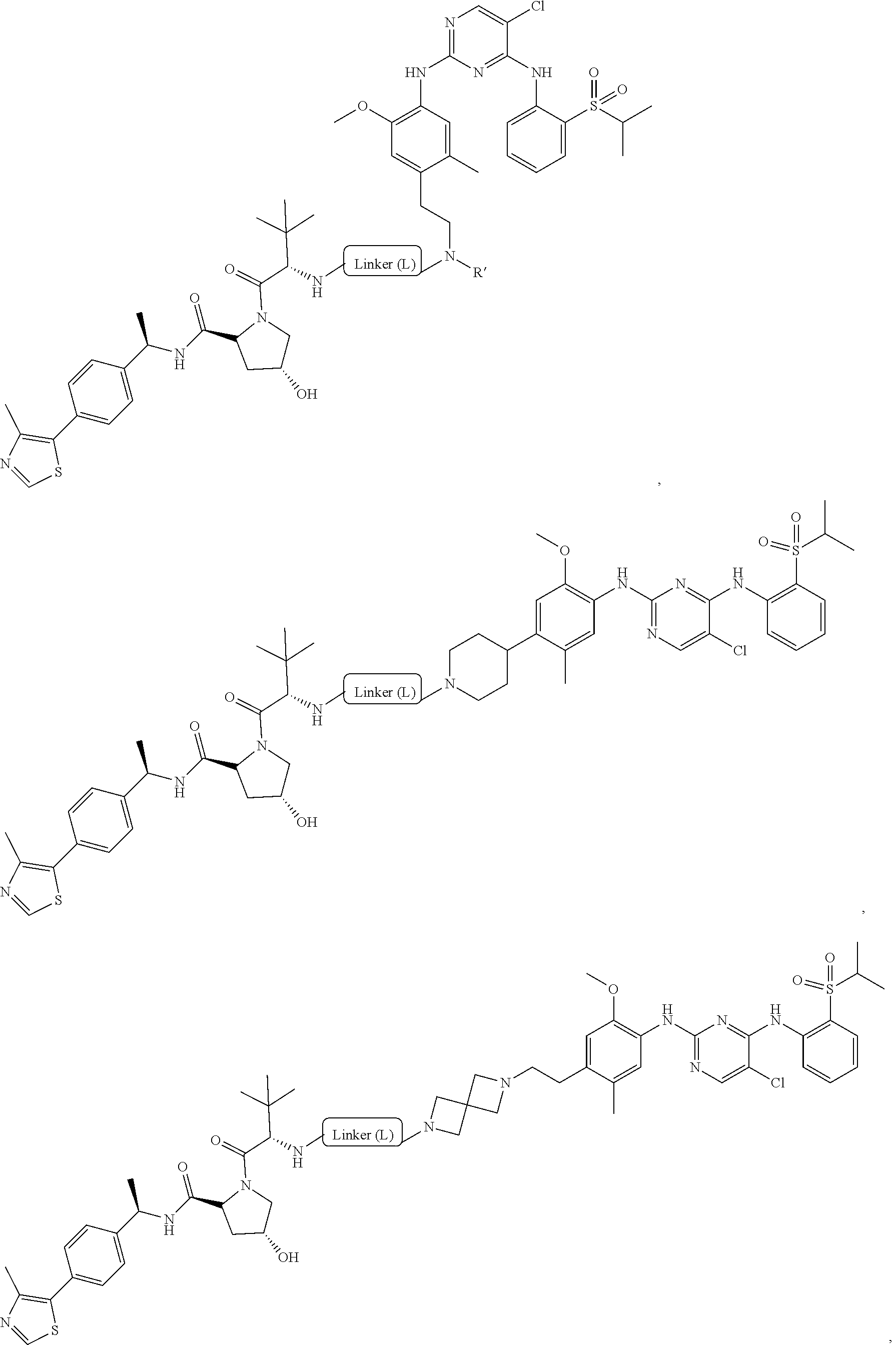

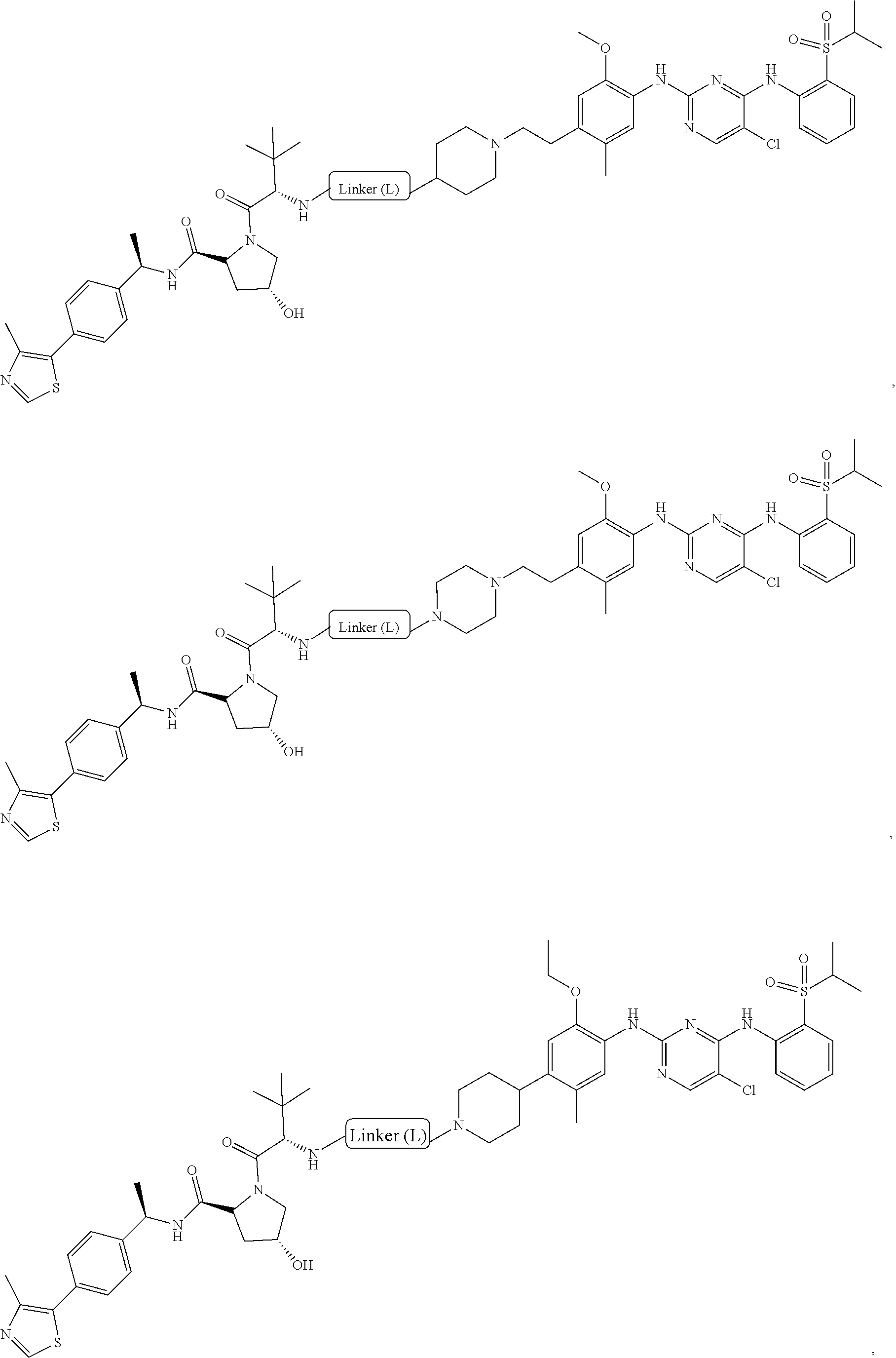

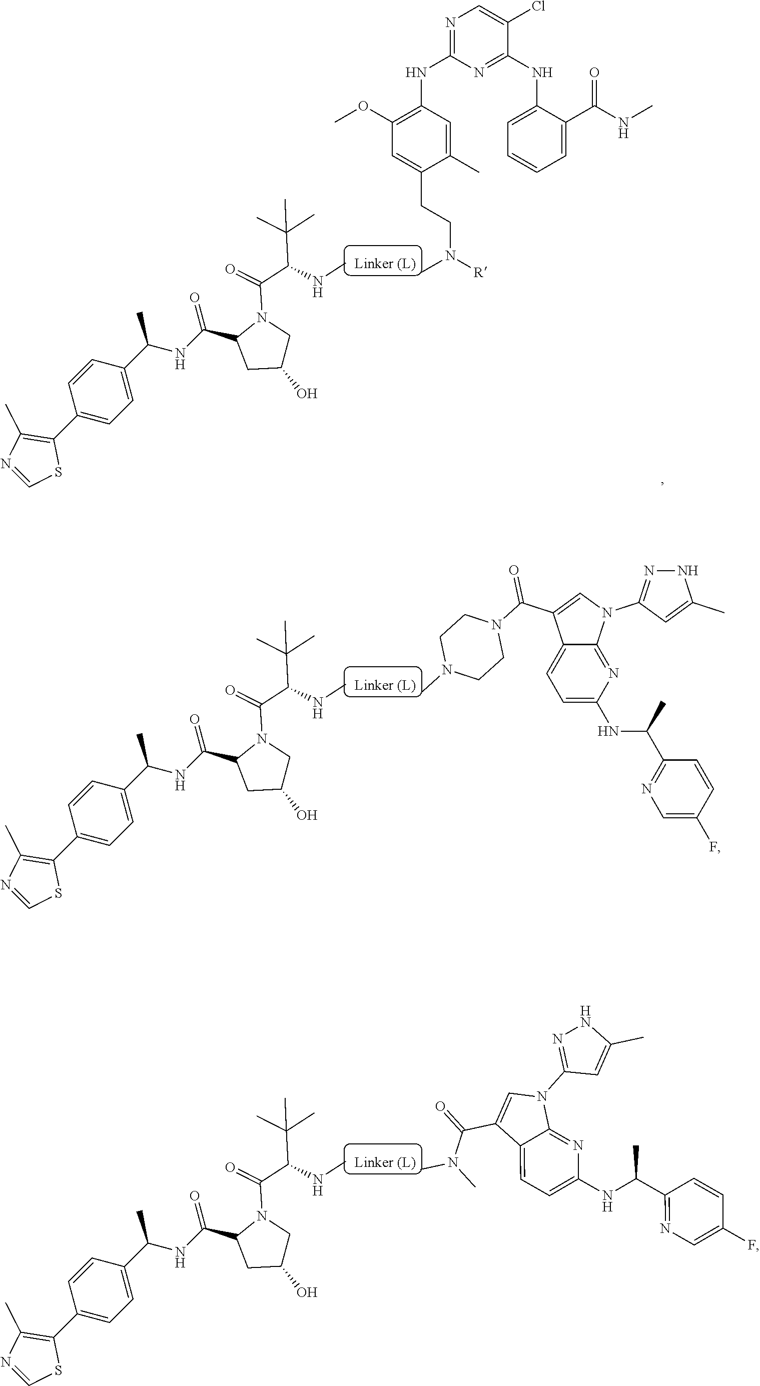

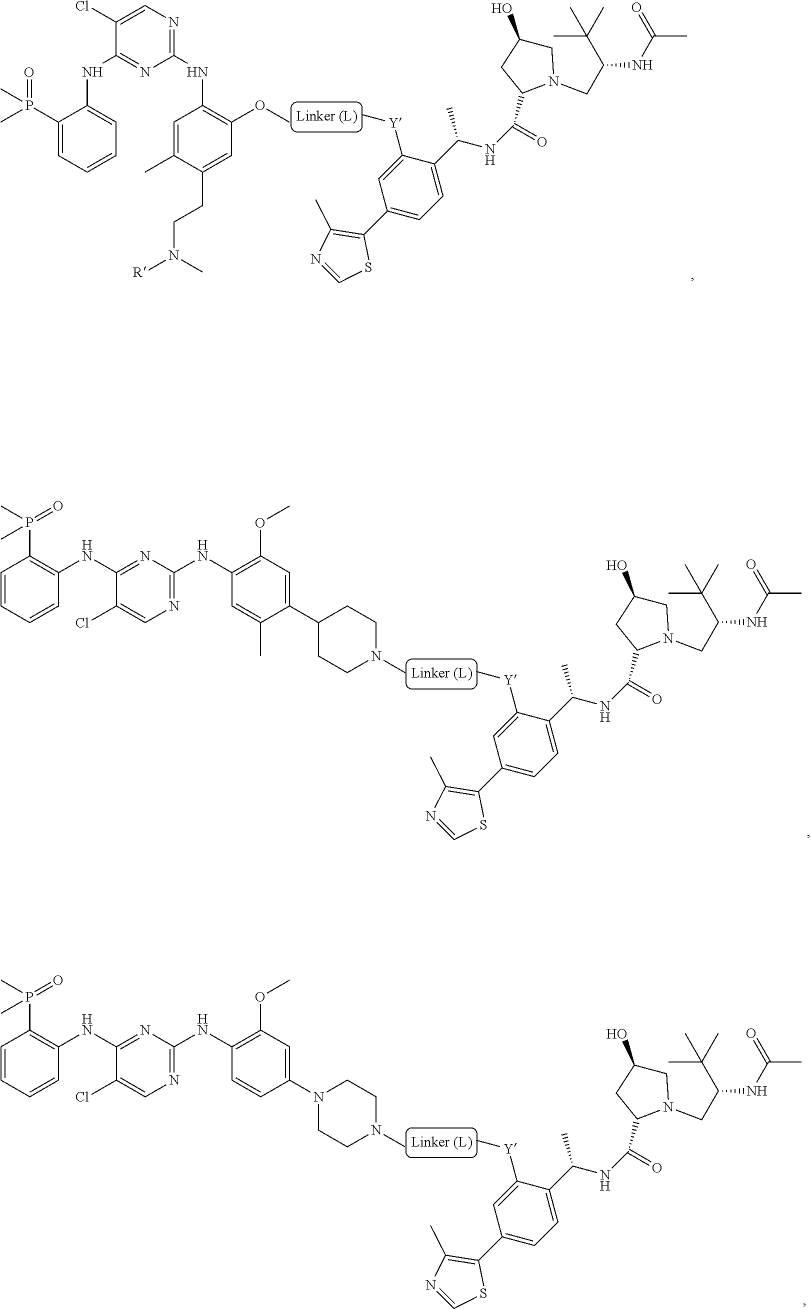

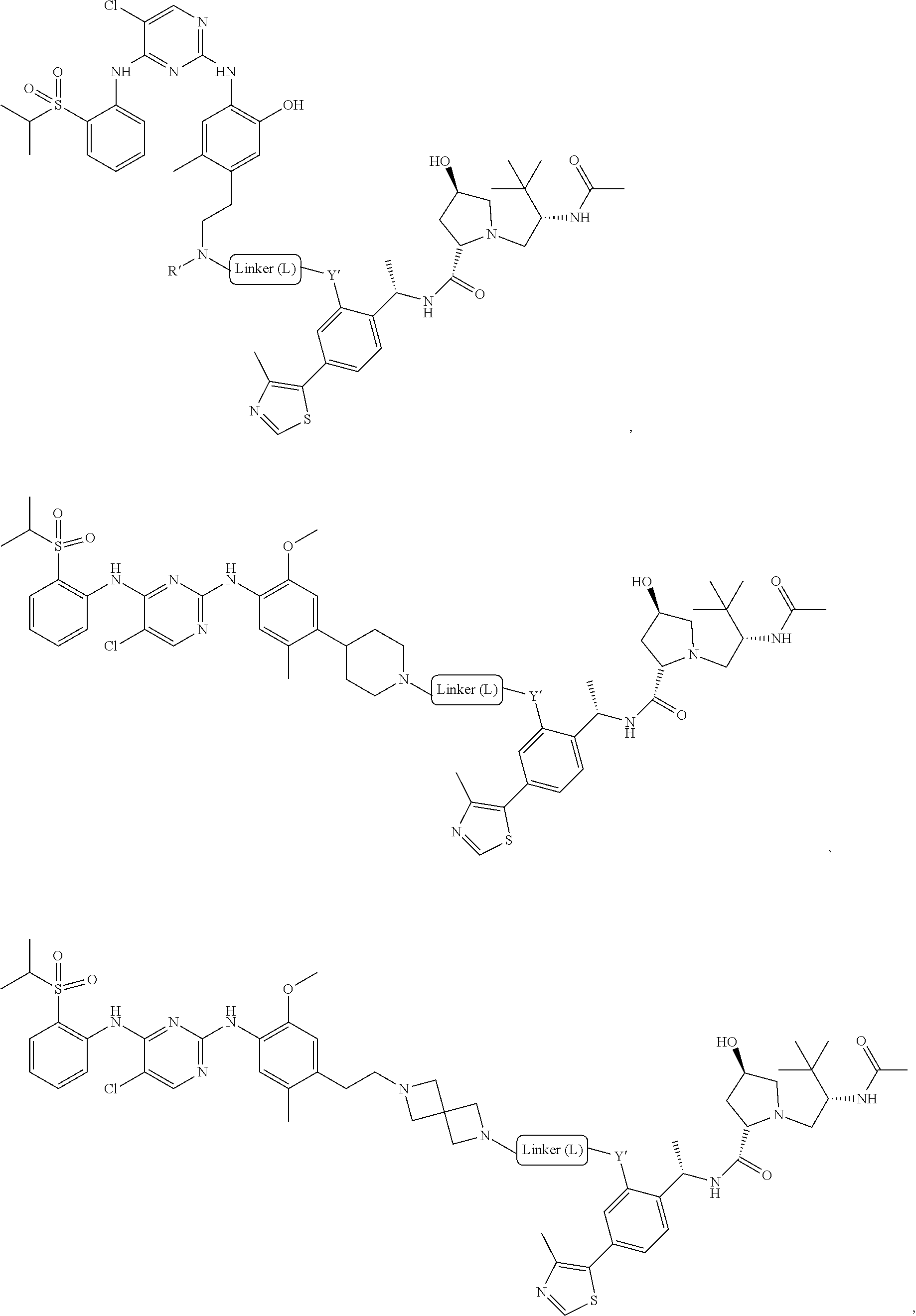

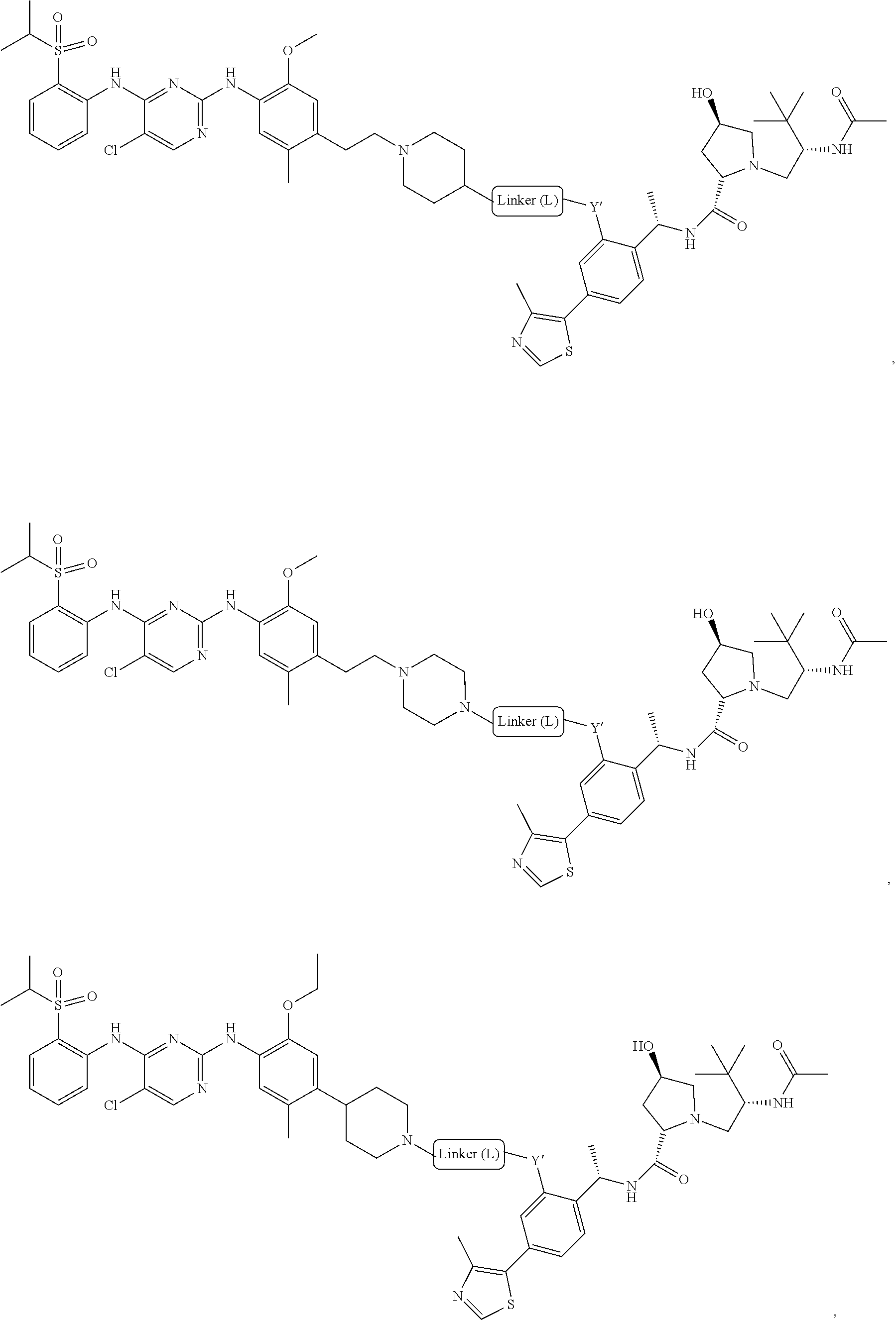

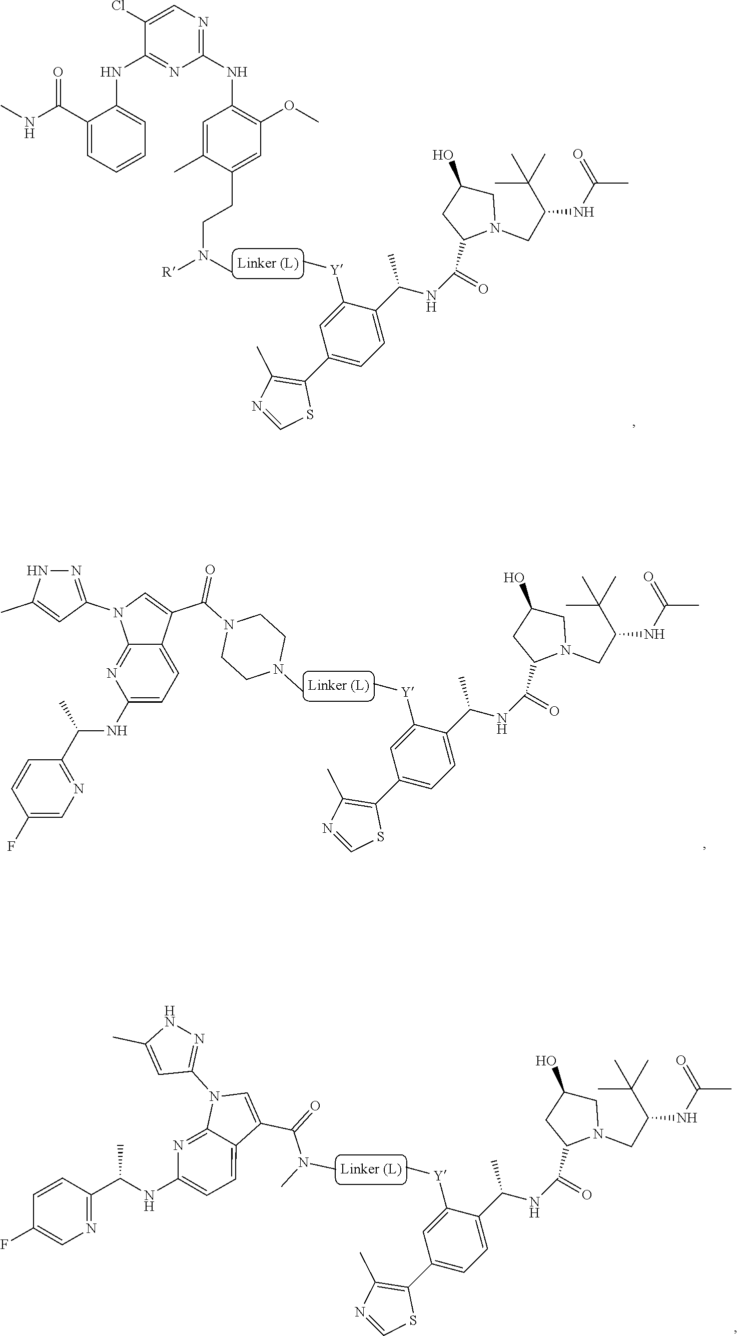

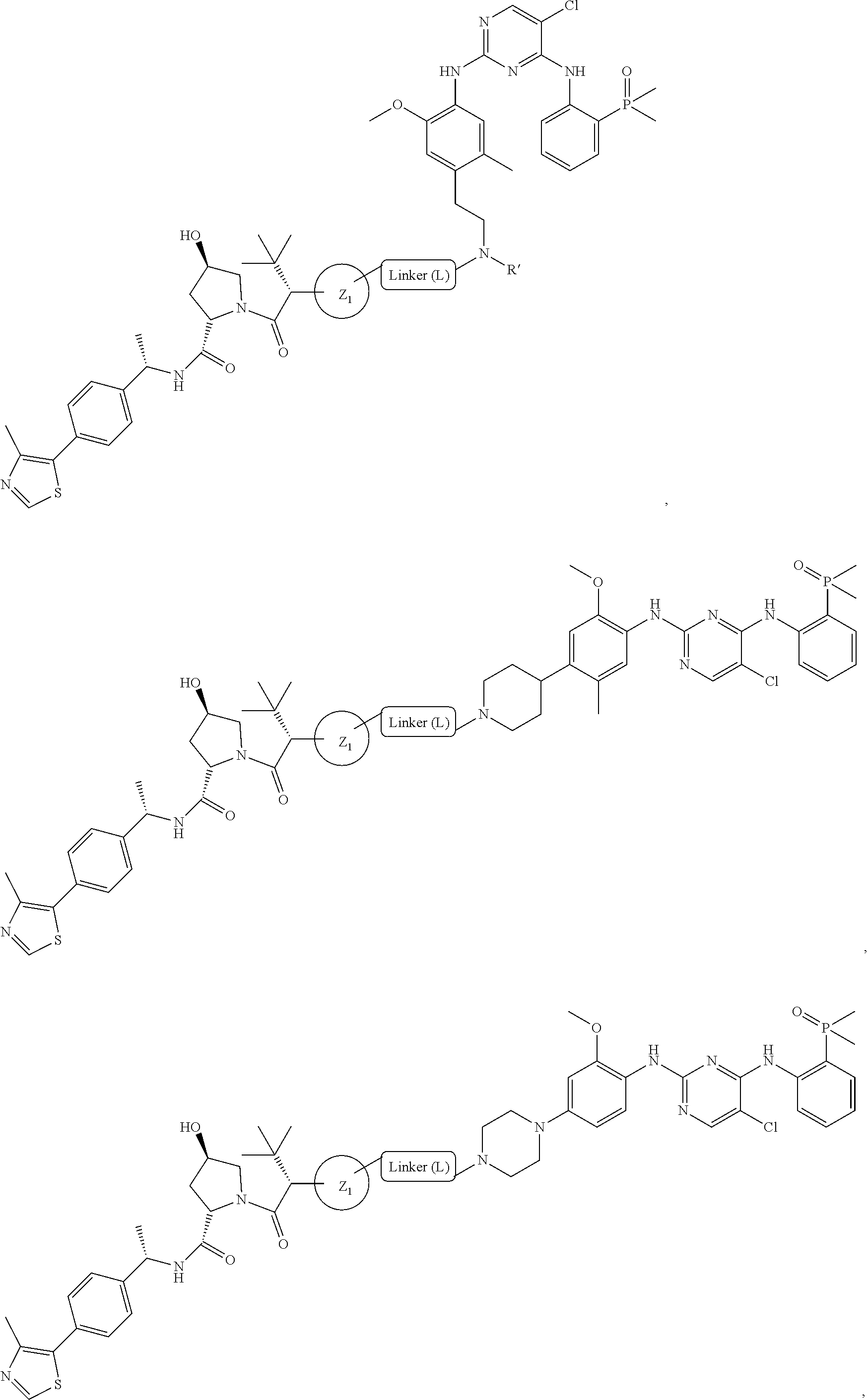

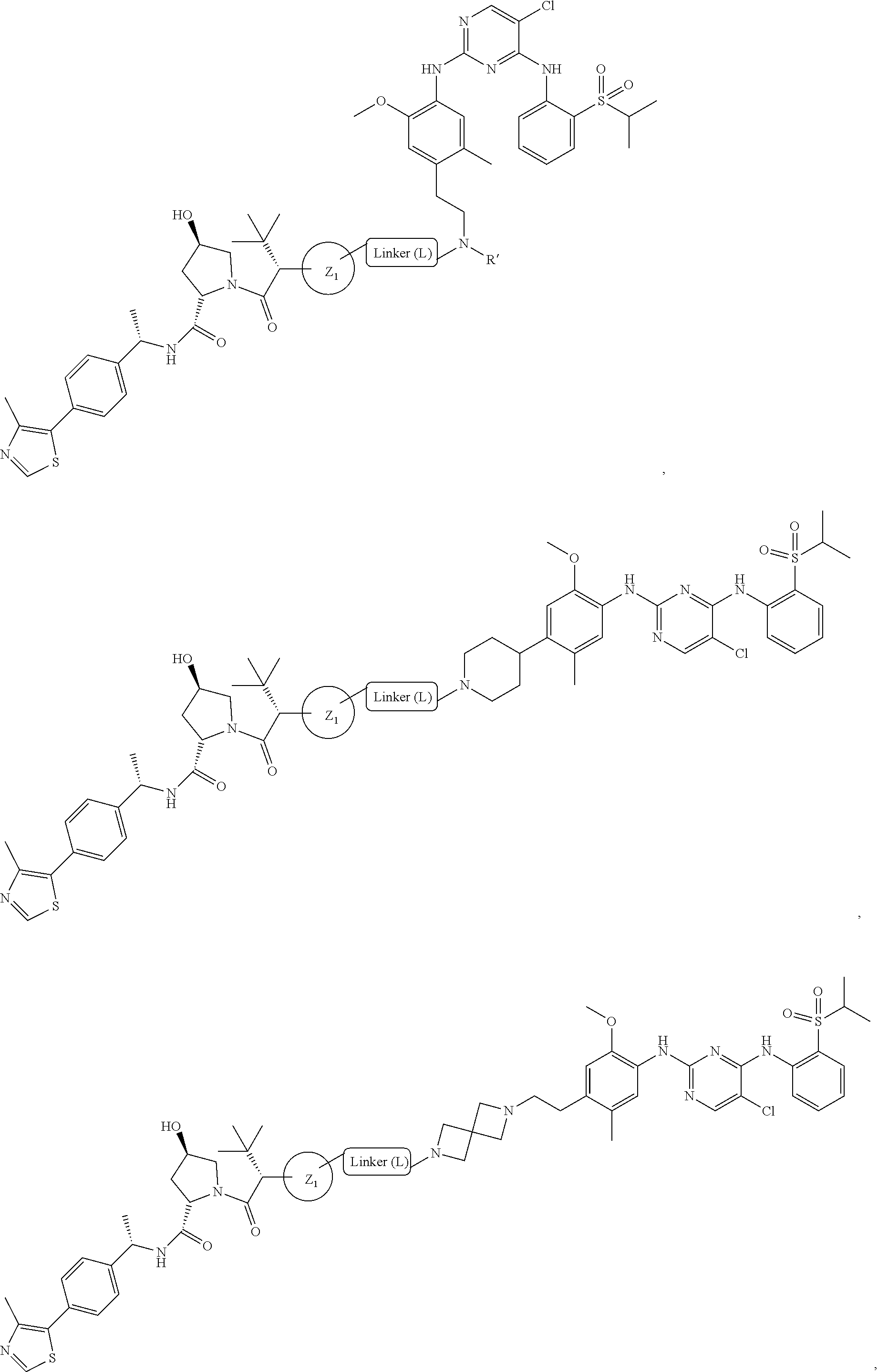

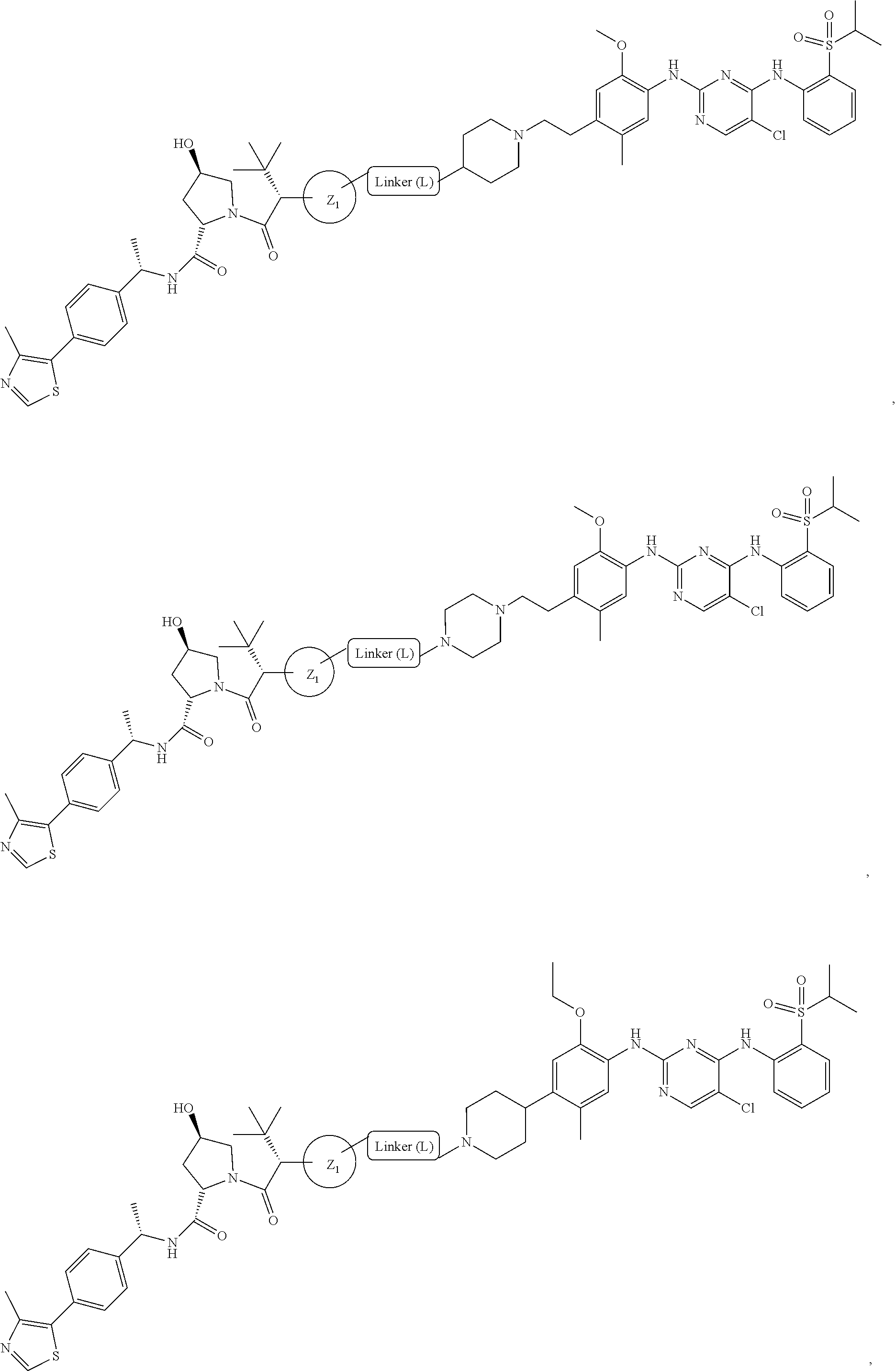

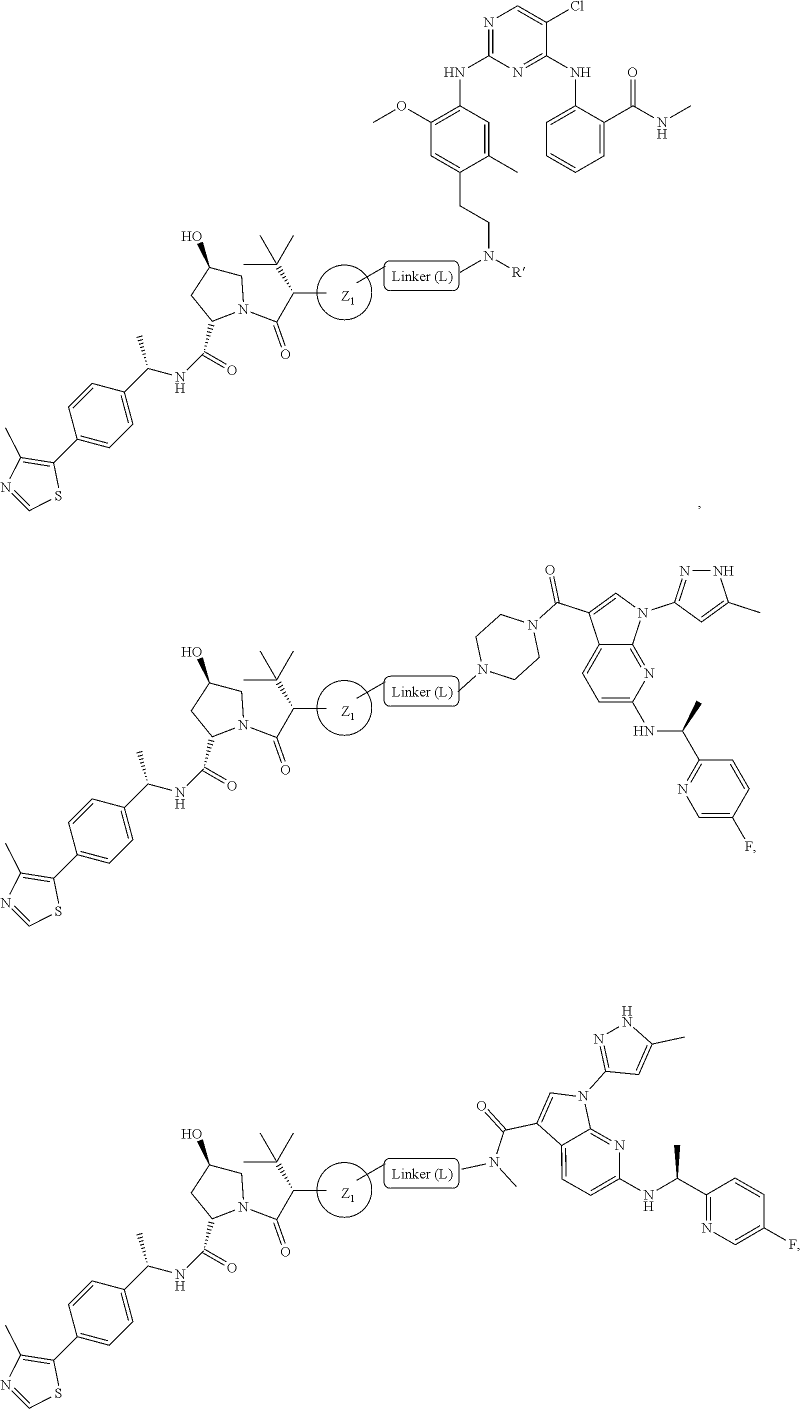

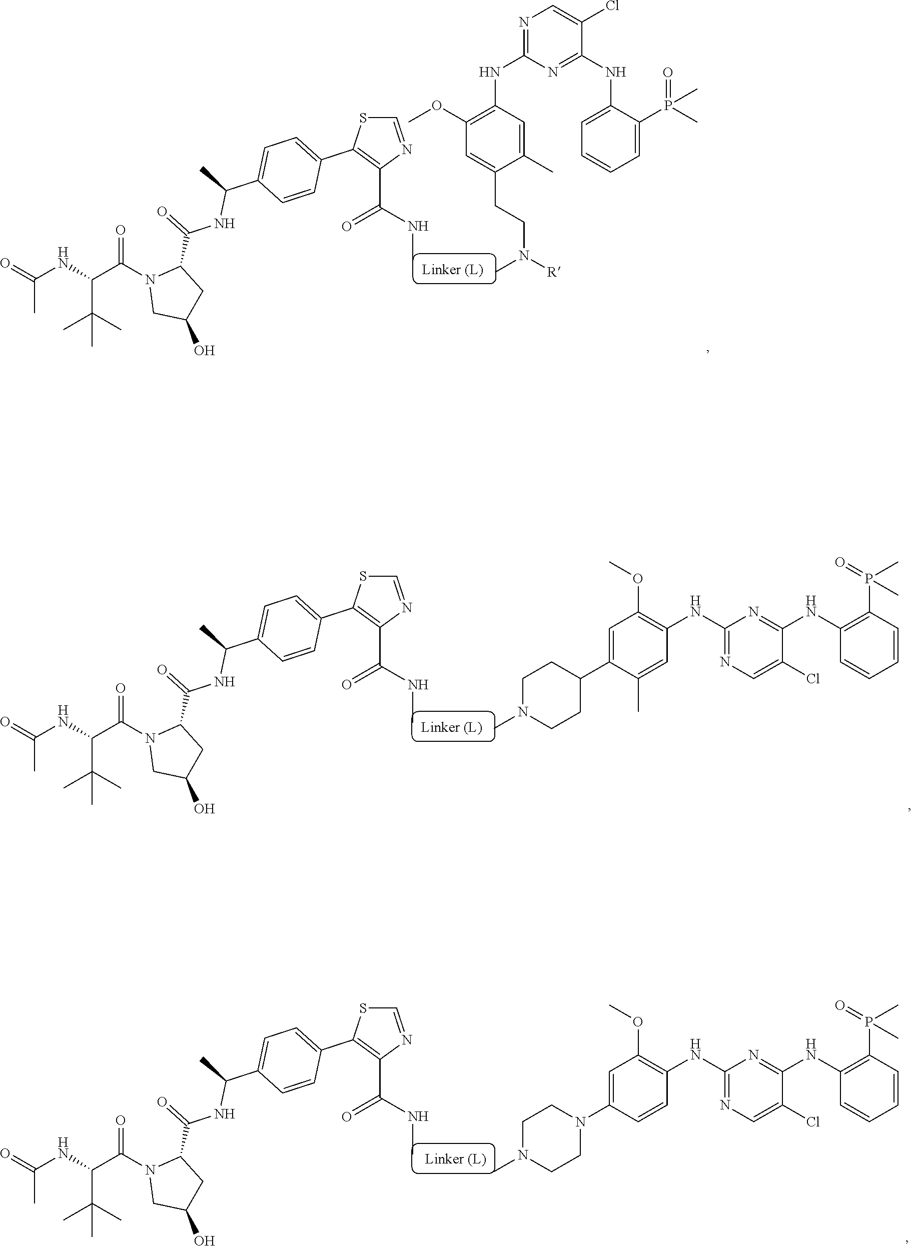

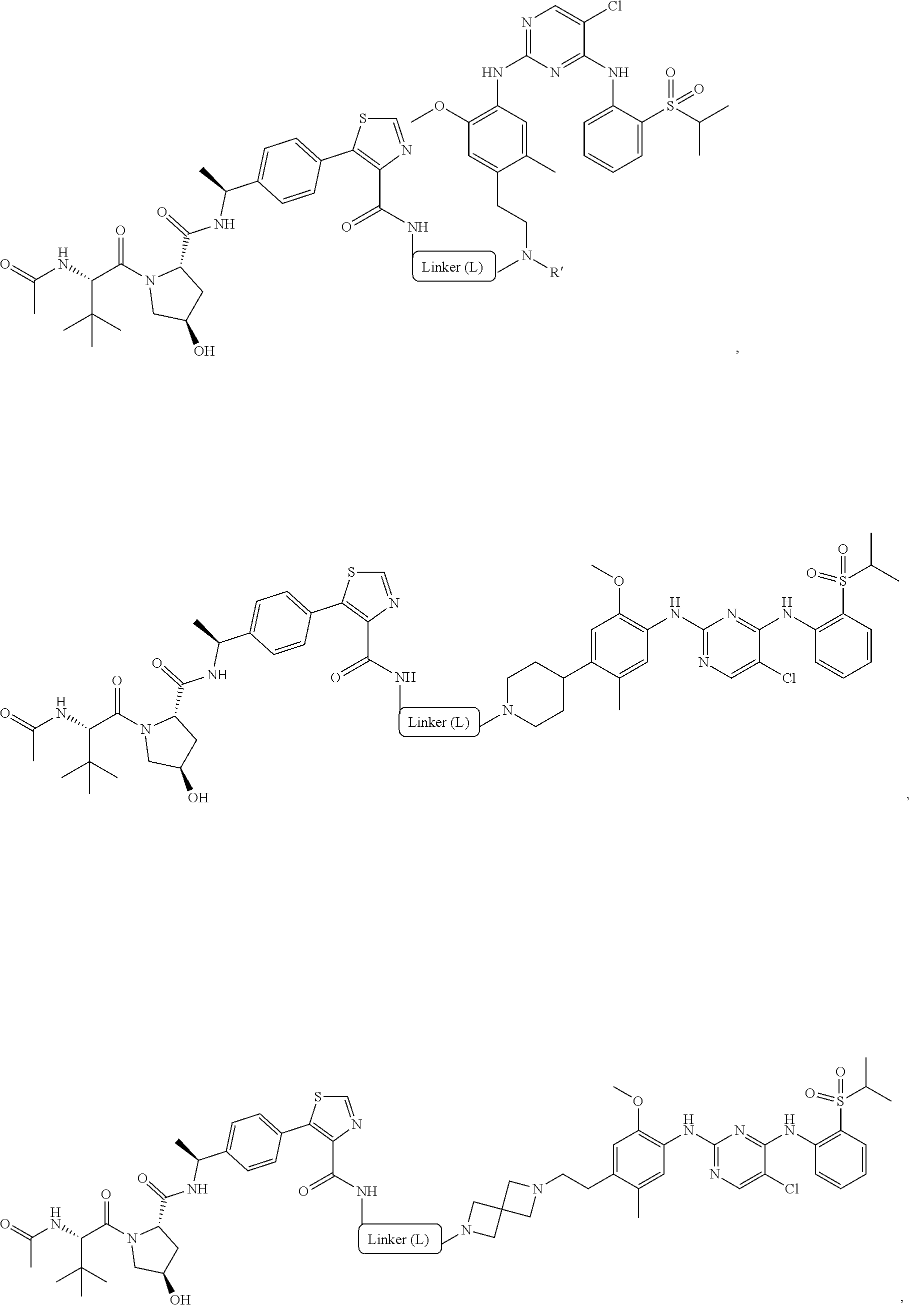

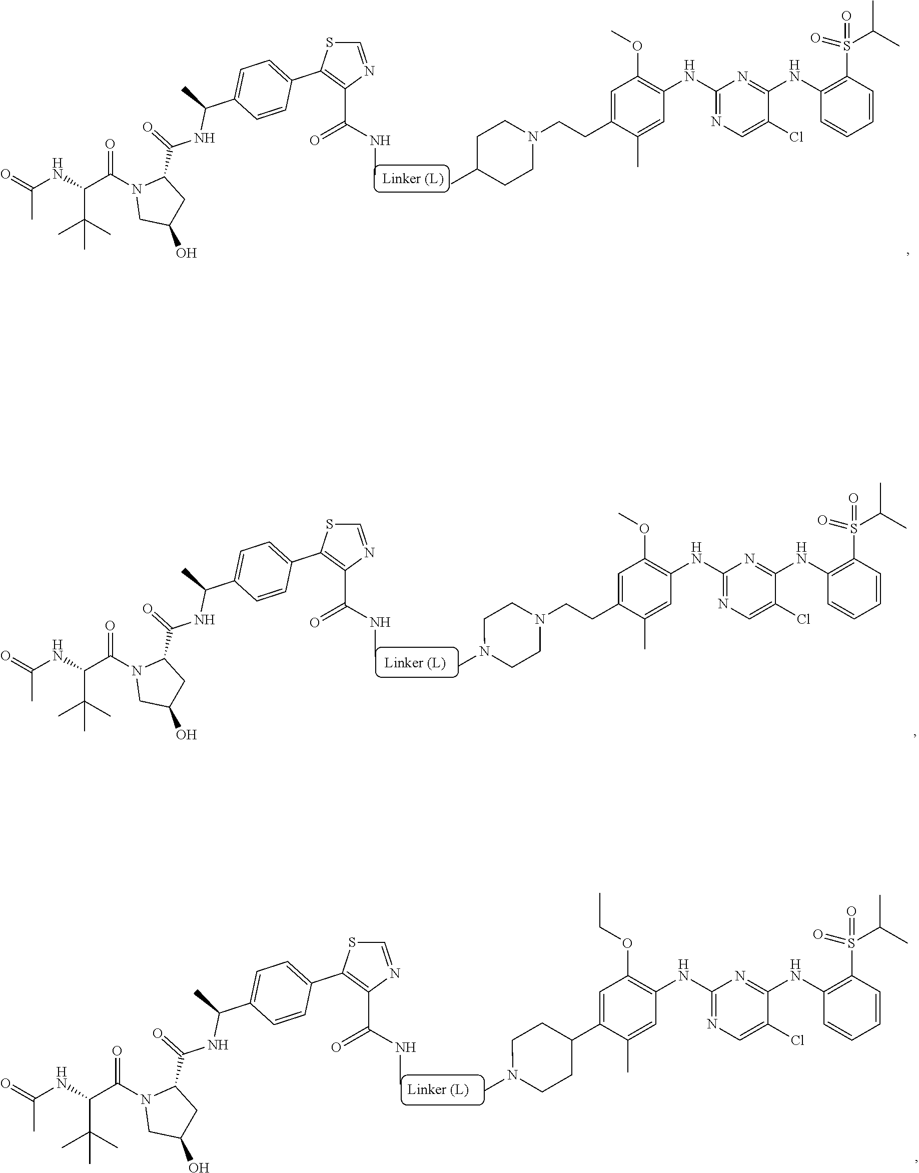

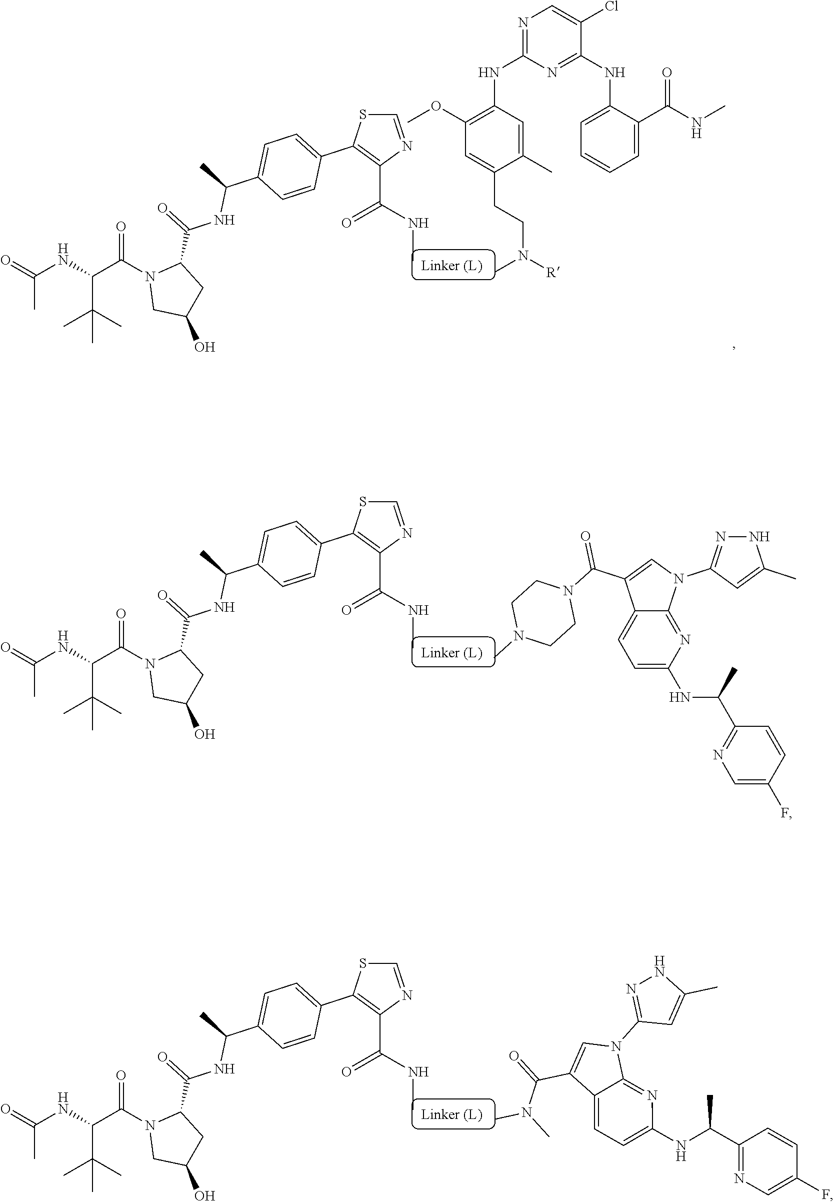

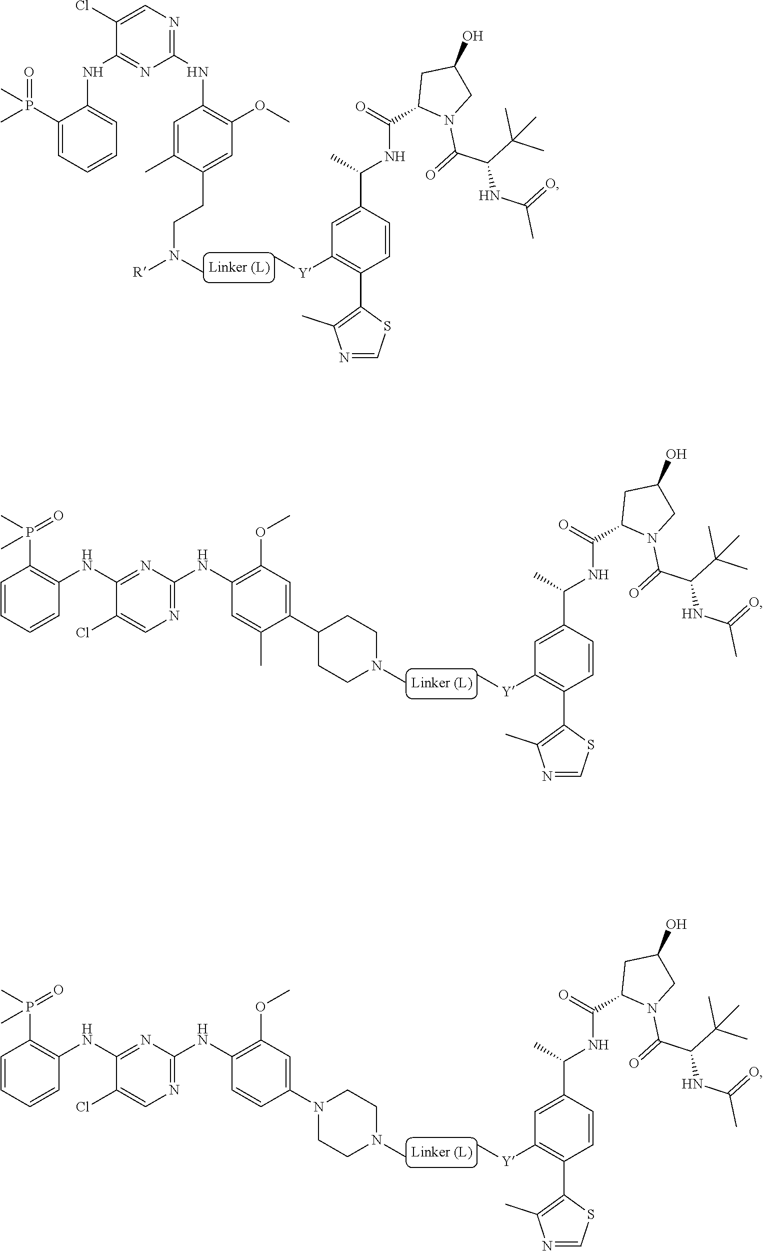

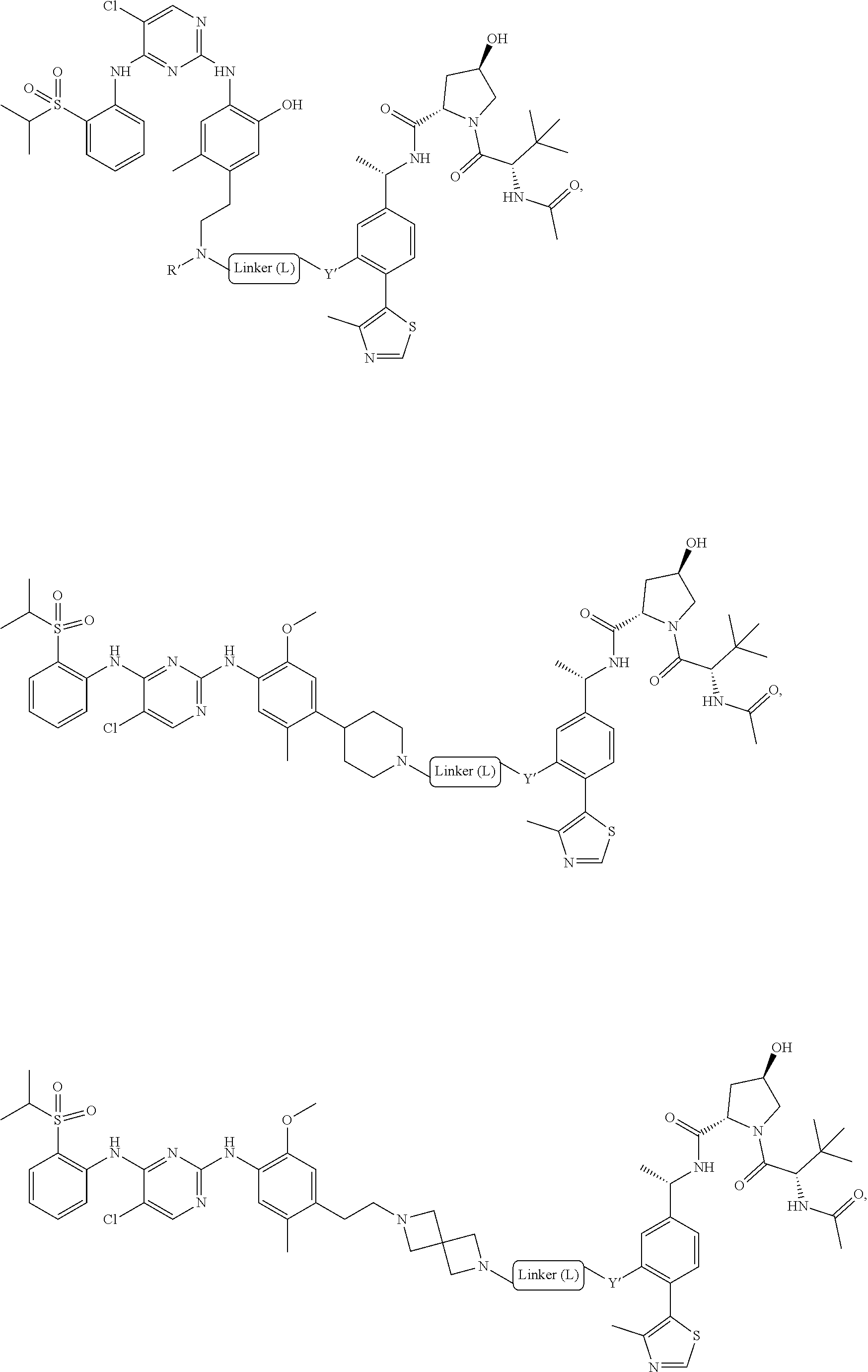

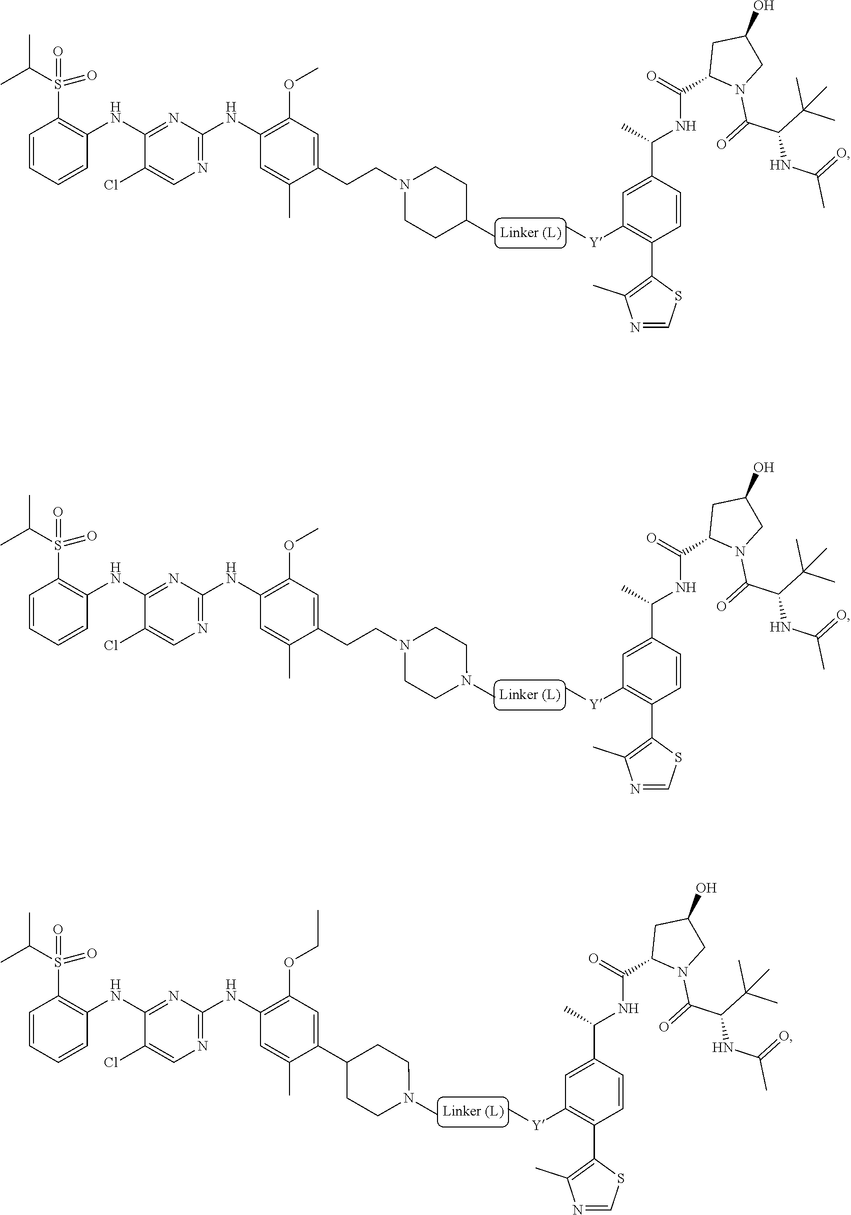

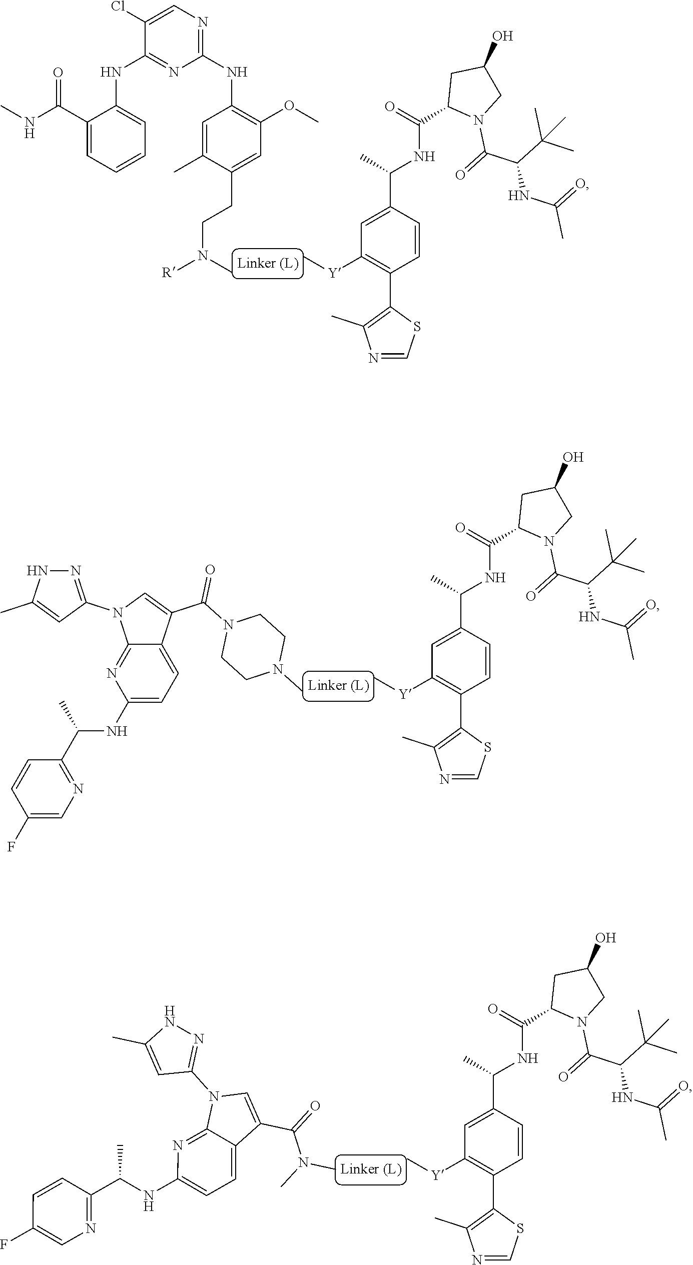

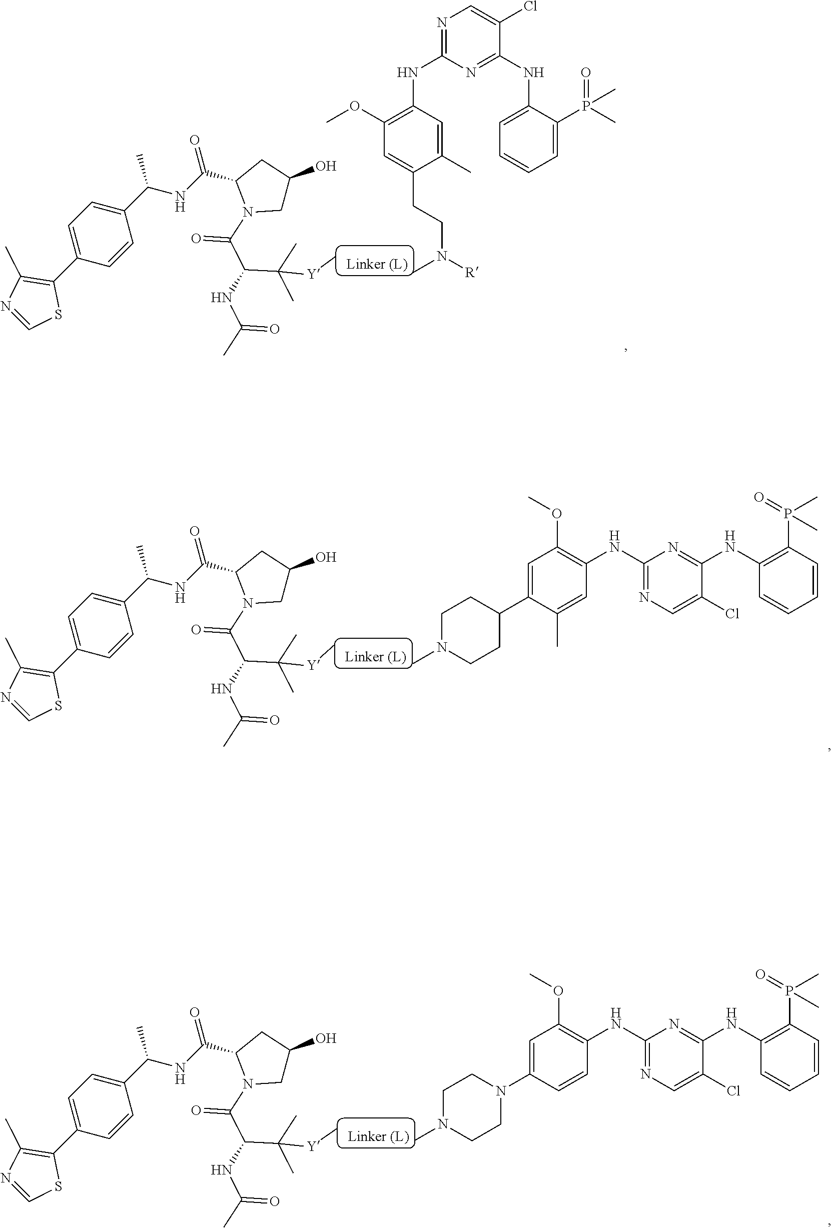

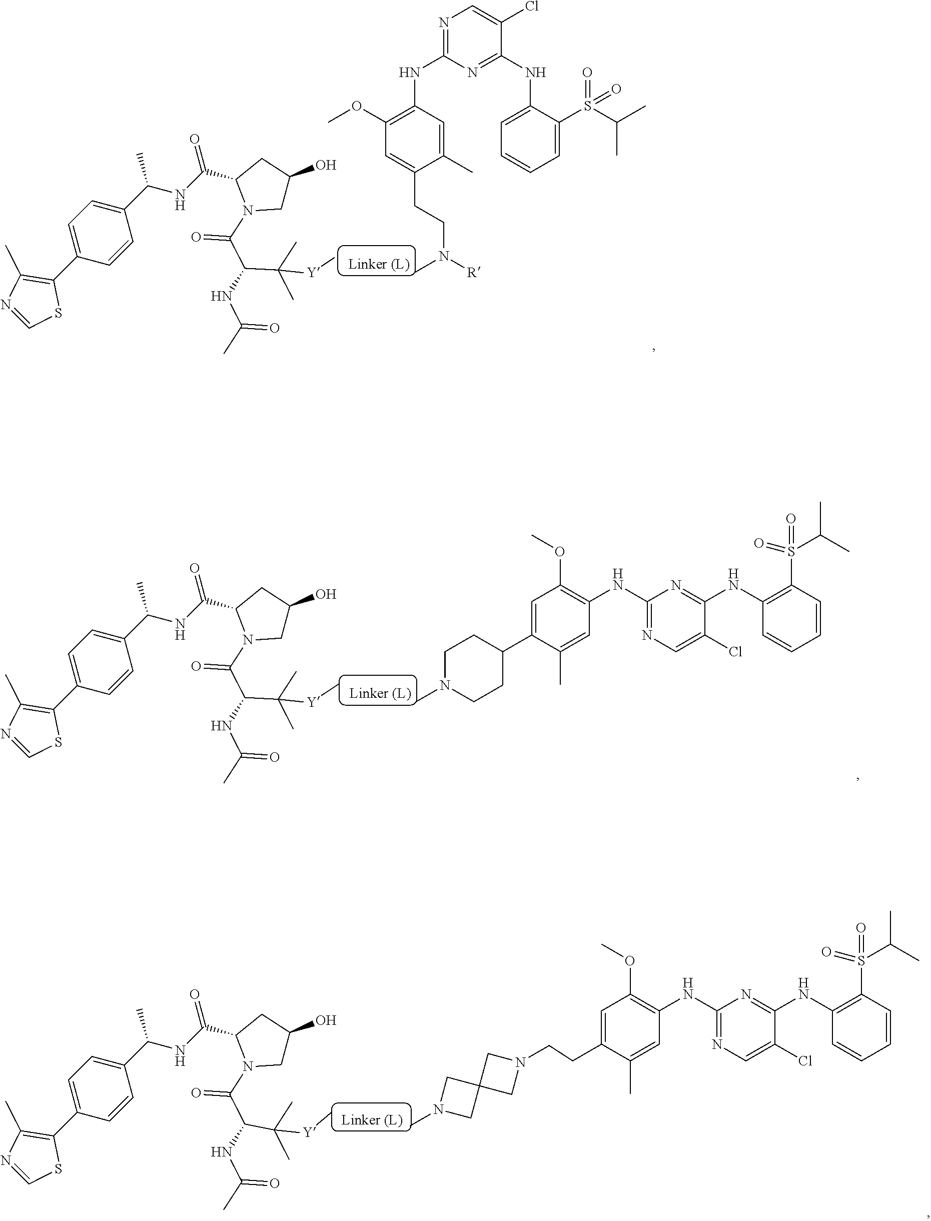

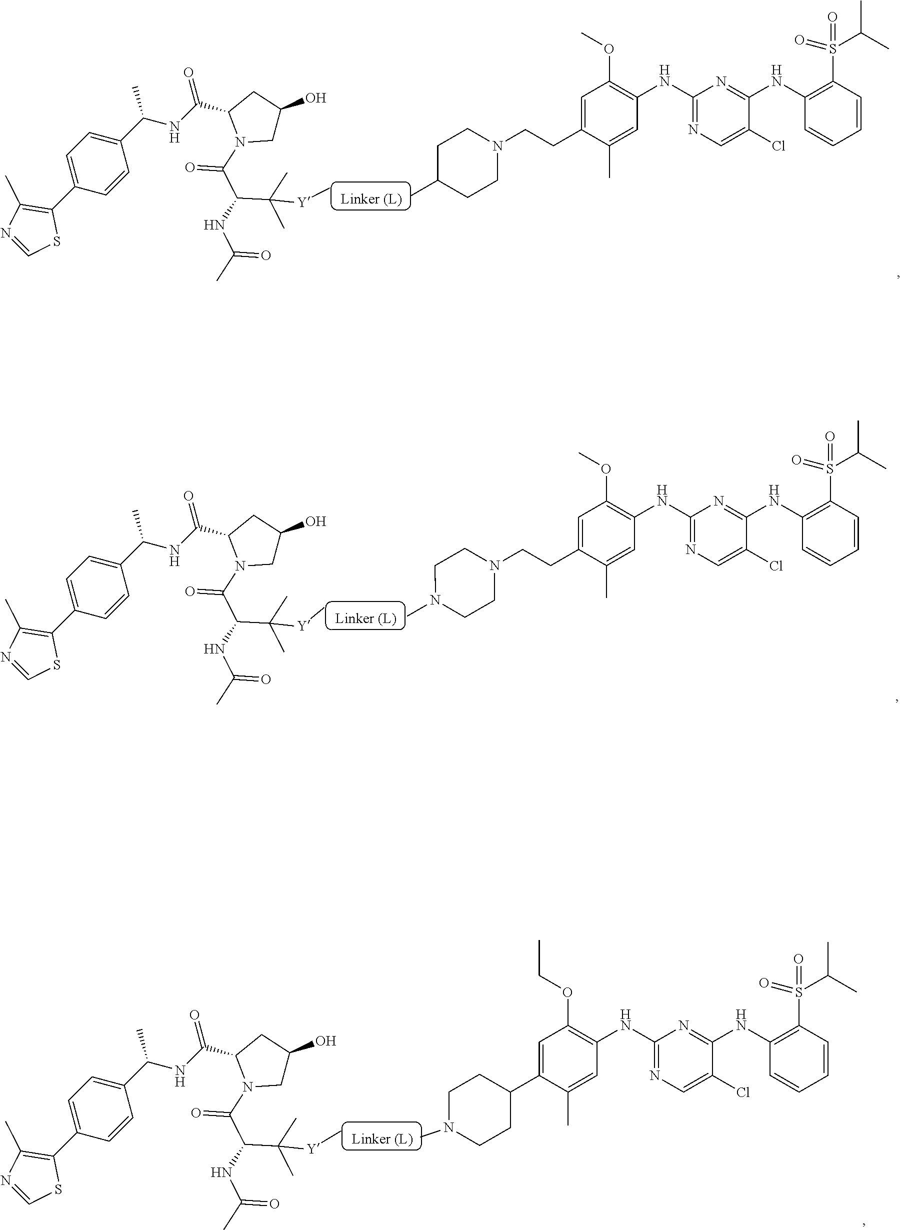

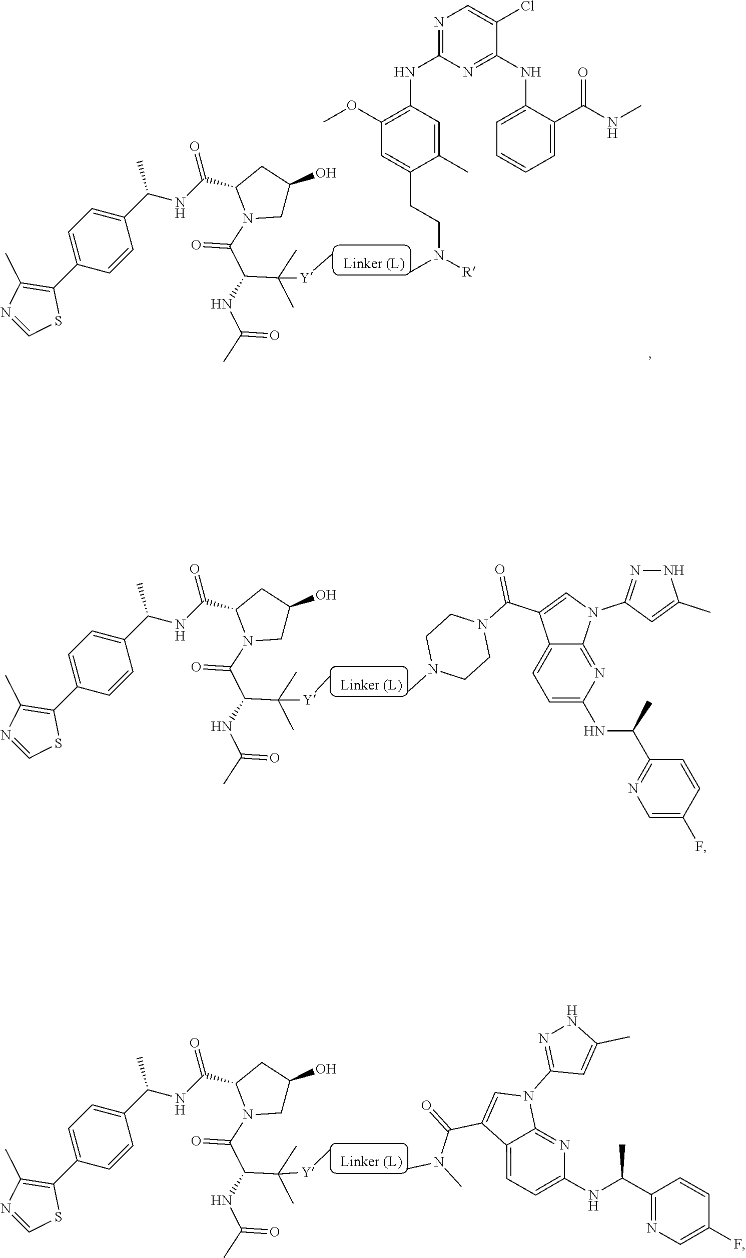

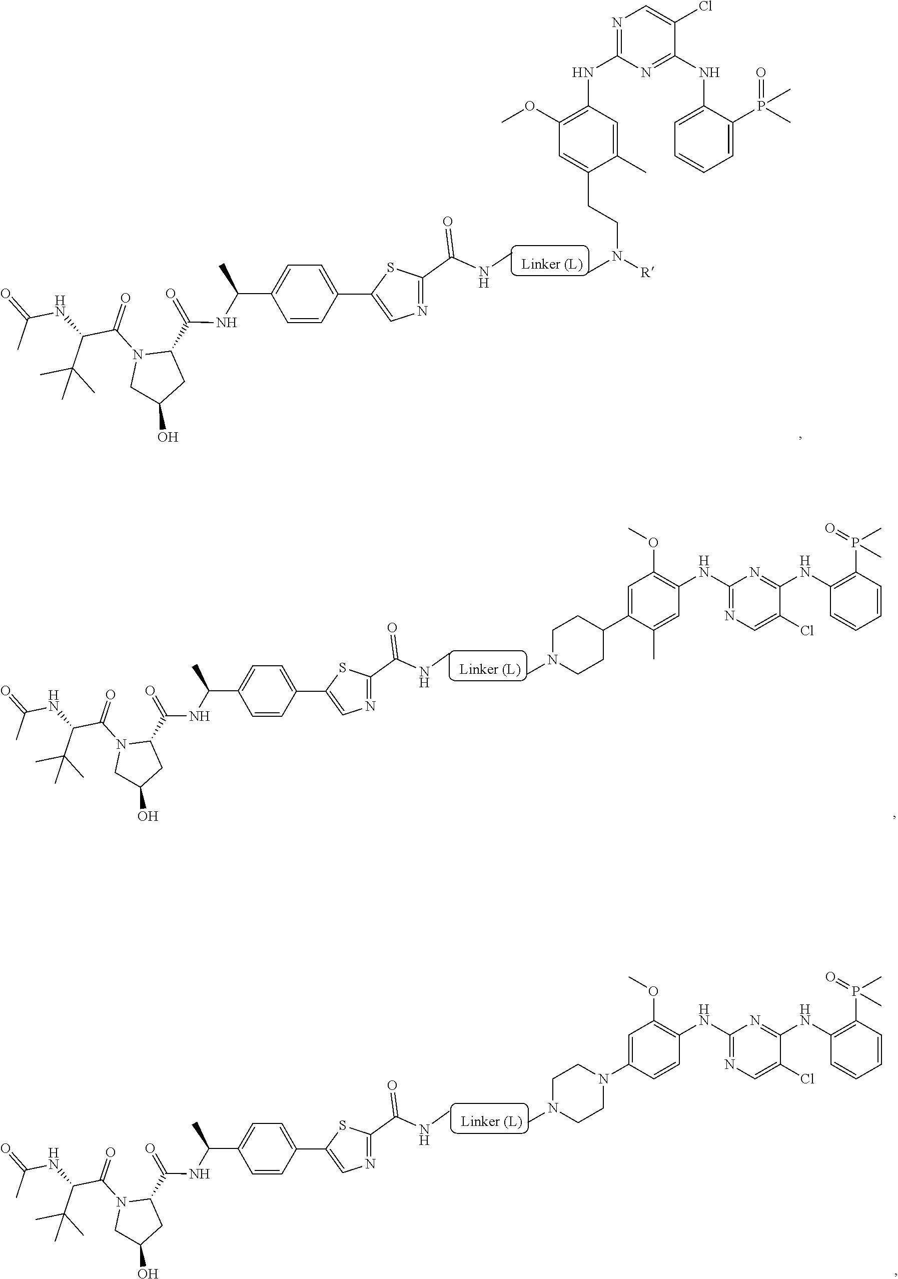

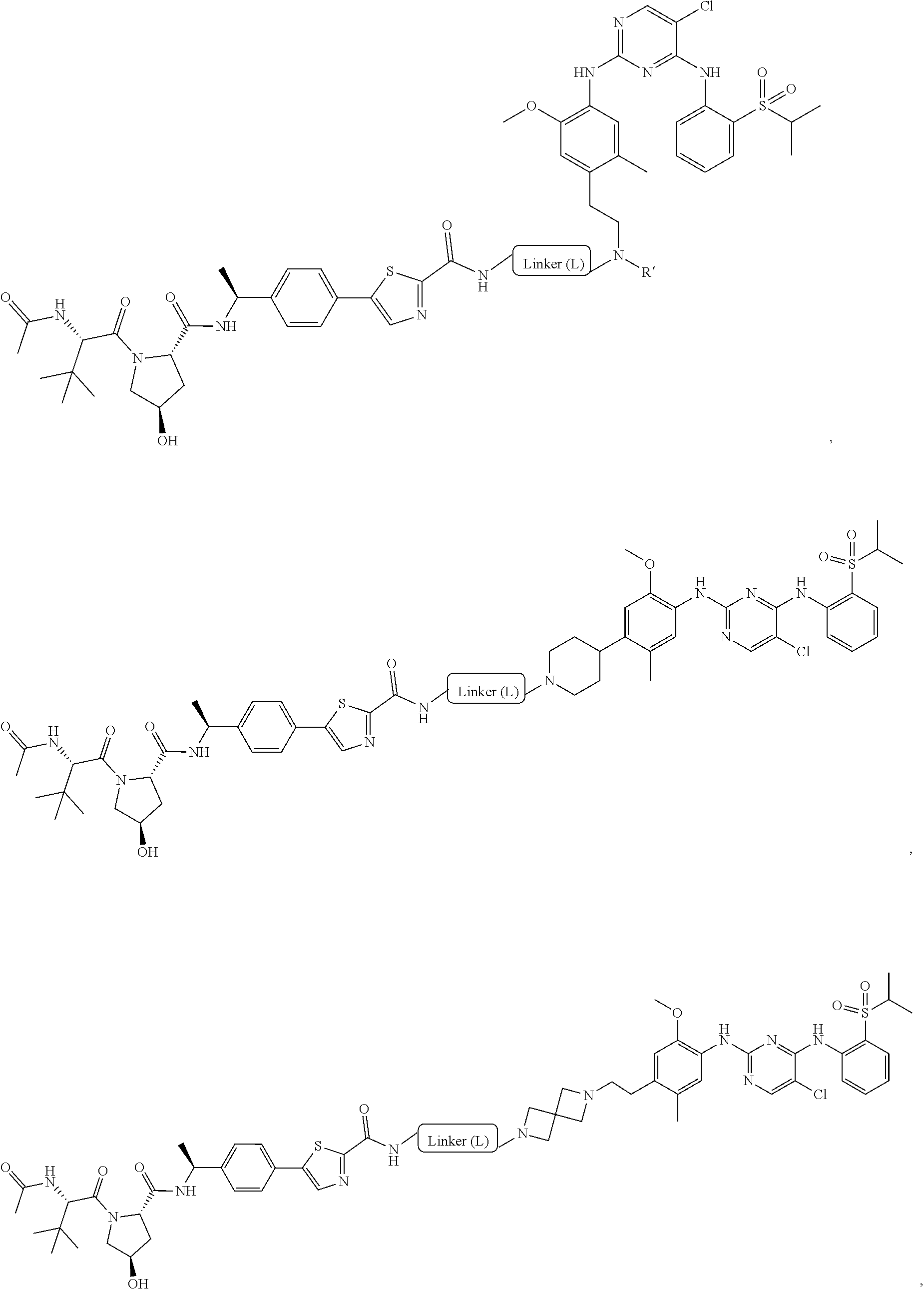

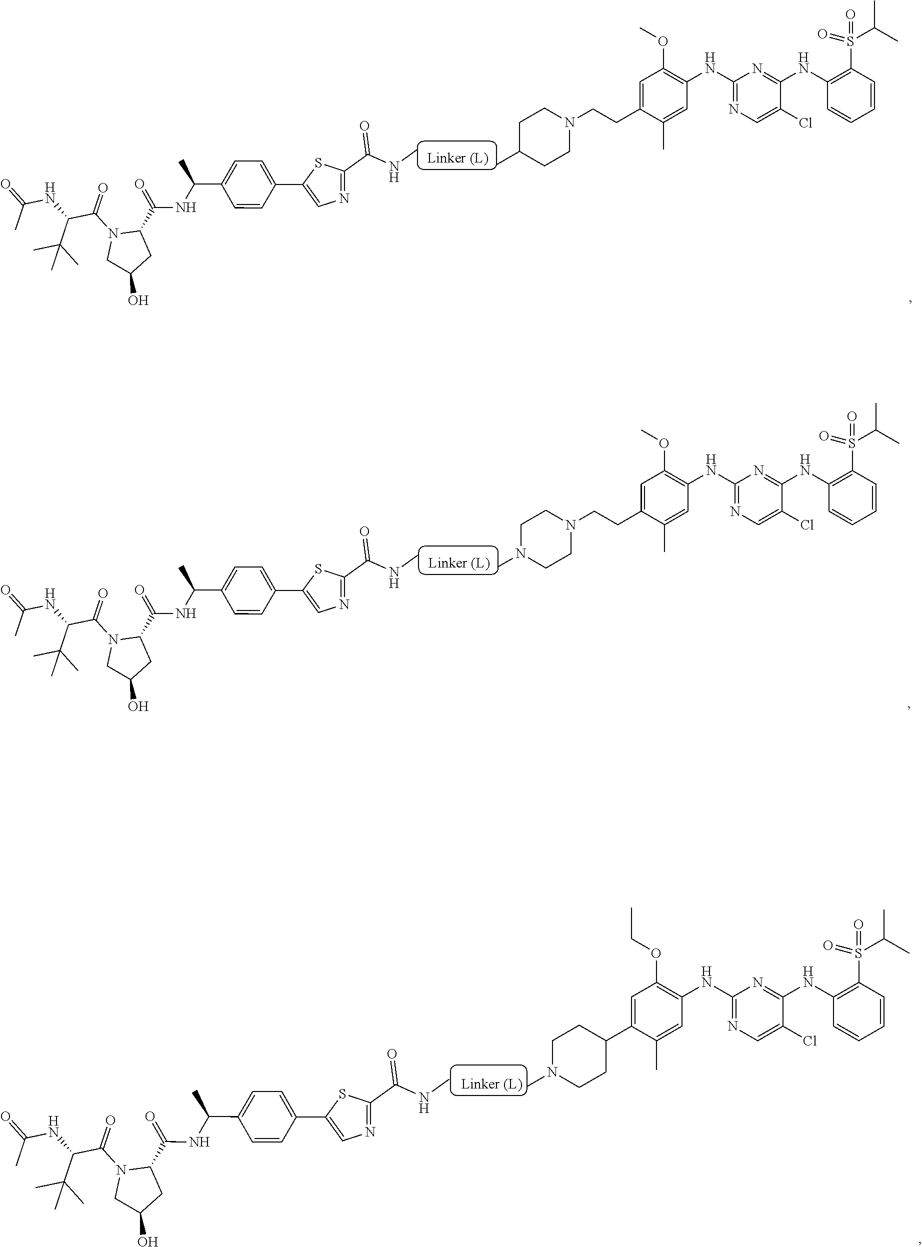 [SMILES: CNC(=O)c1ncc(-c2ccc([C@H](C)NC(=O)[C@@H]3C[C@@H](O)CN3C(=O)[C@@H](NC(C)=O)C(C)(C)C)cc2)s1 ; COc1cc(CCN2CCC(C)CC2)c(C)cc1Nc1ncc(Cl)c(Nc2ccccc2S(=O)(=O)C(C)C)n1 ; CNC(=O)c1ncc(-c2ccc([C@H](C)NC(=O)[C@@H]3C[C@@H](O)CN3C(=O)[C@@H](NC(C)=O)C(C)(C)C)cc2)s1 ; COc1cc(CCN2CCN(C)CC2)c(C)cc1Nc1ncc(Cl)c(Nc2ccccc2S(=O)(=O)C(C)C)n1 ; CNC(=O)c1ncc(-c2ccc([C@H](C)NC(=O)[C@@H]3C[C@@H](O)CN3C(=O)[C@@H](NC(C)=O)C(C)(C)C)cc2)s1 ; CCOc1cc(C2CCN(C)CC2)c(C)cc1Nc1ncc(Cl)c(Nc2ccccc2S(=O)(=O)C(C)C)n1]

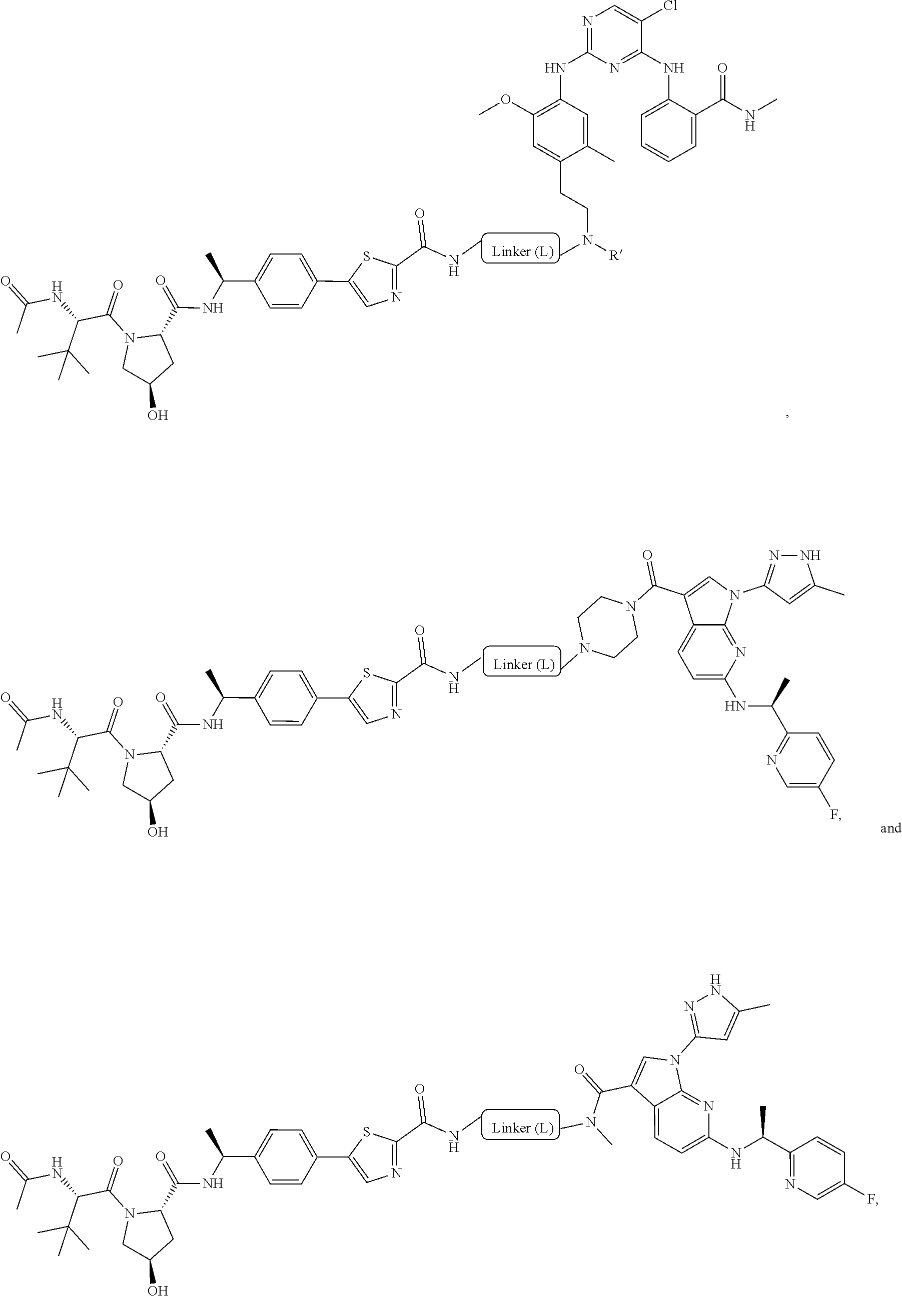

or a pharmaceutically acceptable salt or stereoisomer thereof.

Yet other degrons that bind VHL and which may be suitable for use in the present invention are disclosed in U.S. Patent Application Publication 2017/0121321 A1.

In some embodiments, the E3 ubiquitin ligase that is bound by the degron is an inhibitor of apoptosis protein (IAP). Representative examples of degrons that bind IAP and may be suitable for use in the present invention are represented by any one of the following structures:

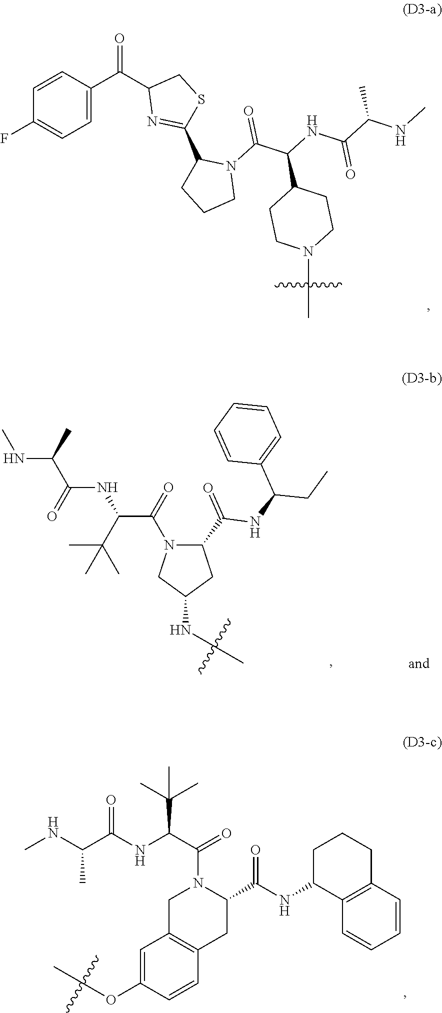

or stereoisomer thereof.

Therefore, in some embodiments, bispecific compounds of the present invention are represented by any one of the following structures:

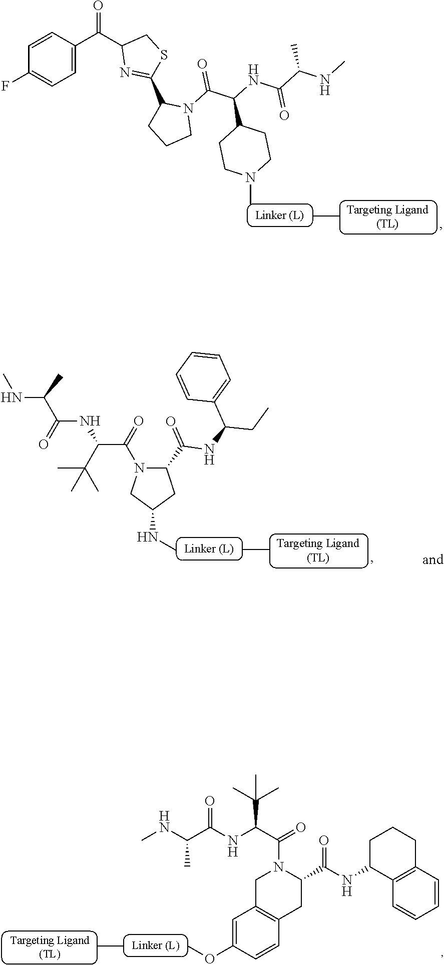

or a pharmaceutically acceptable salt or stereoisomer thereof.

In some embodiments, bispecific compounds of the present invention are represented by any one of the following structures:

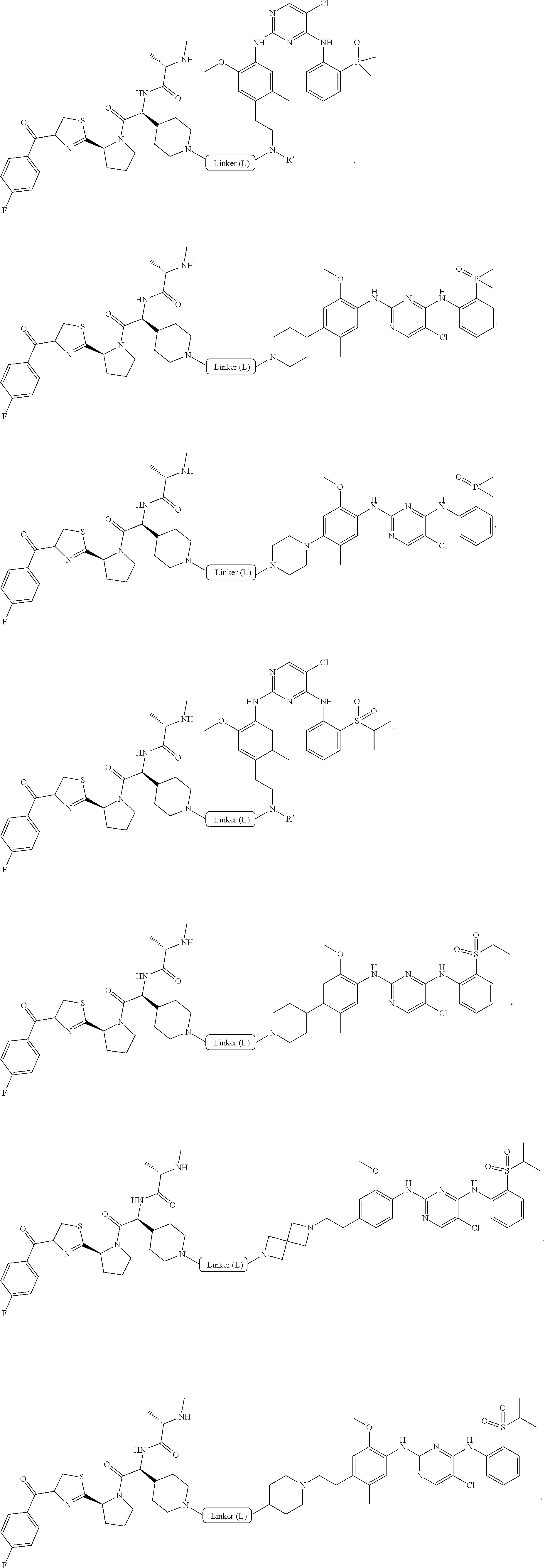

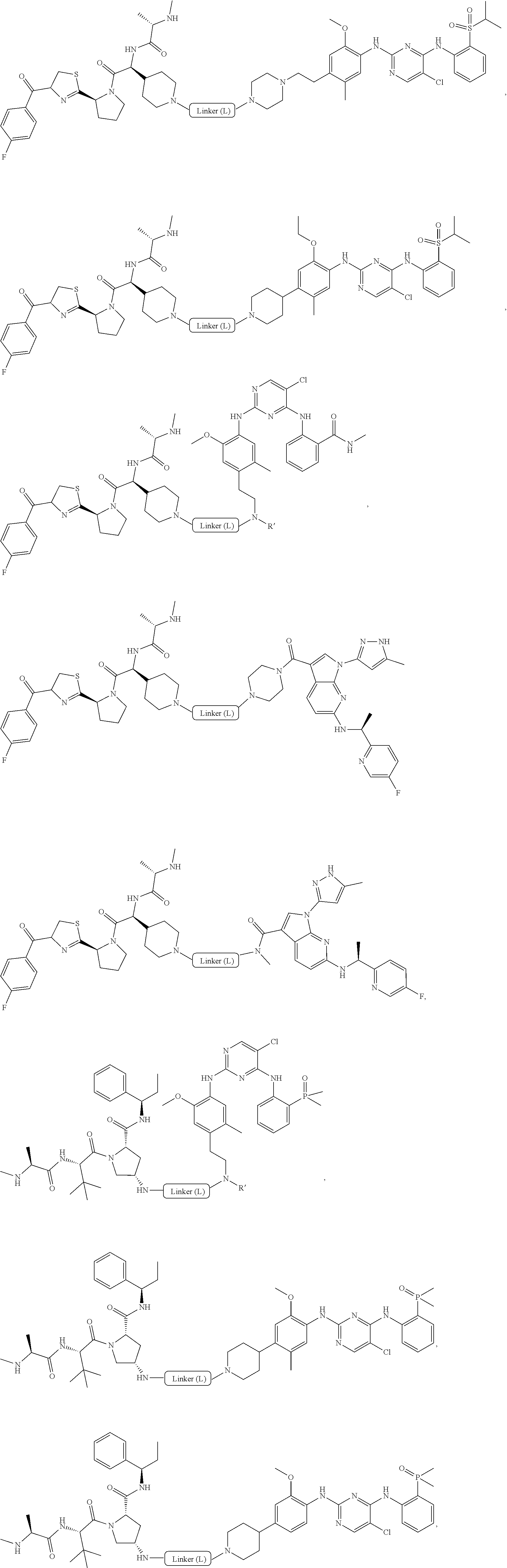

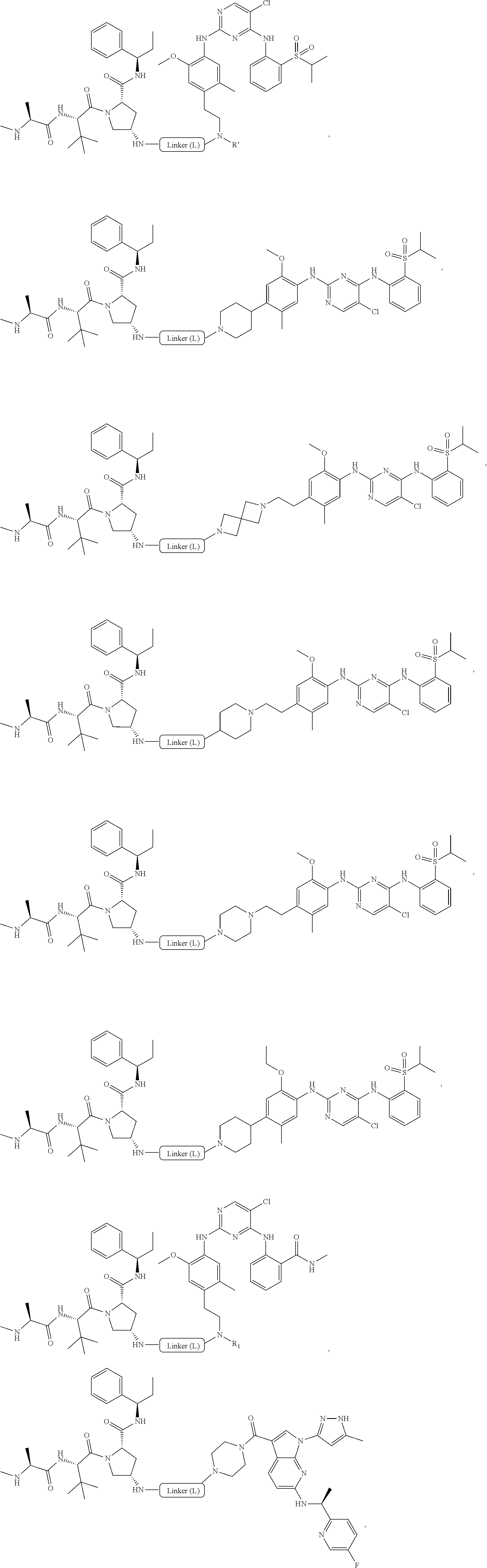

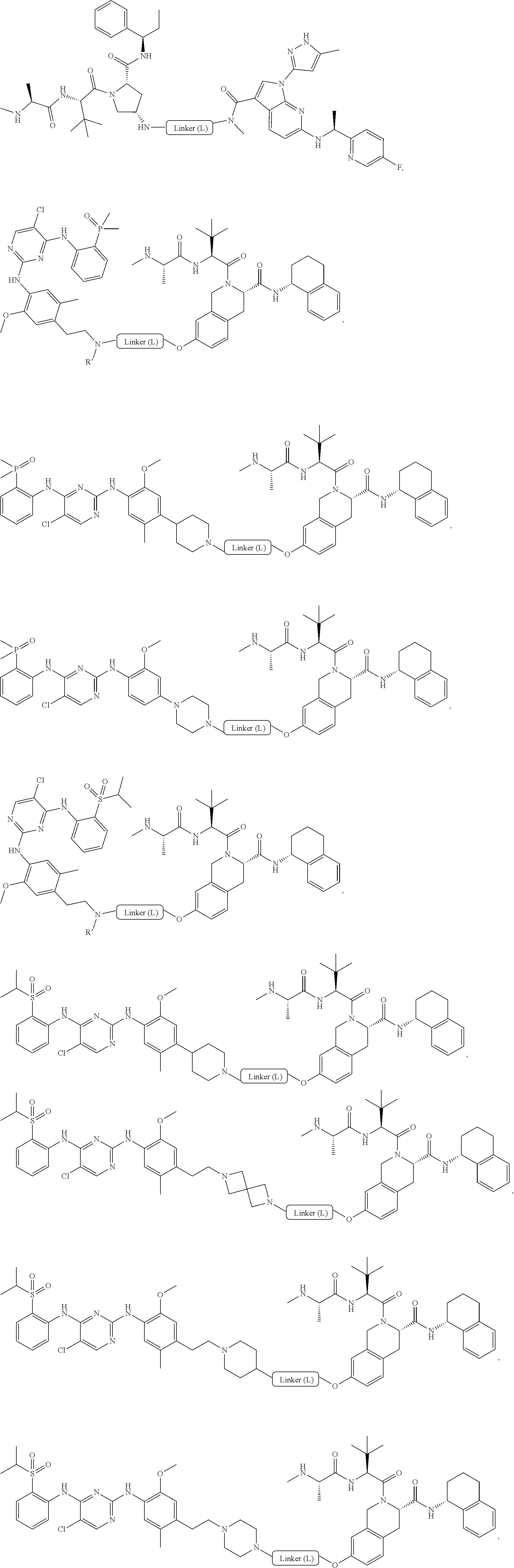

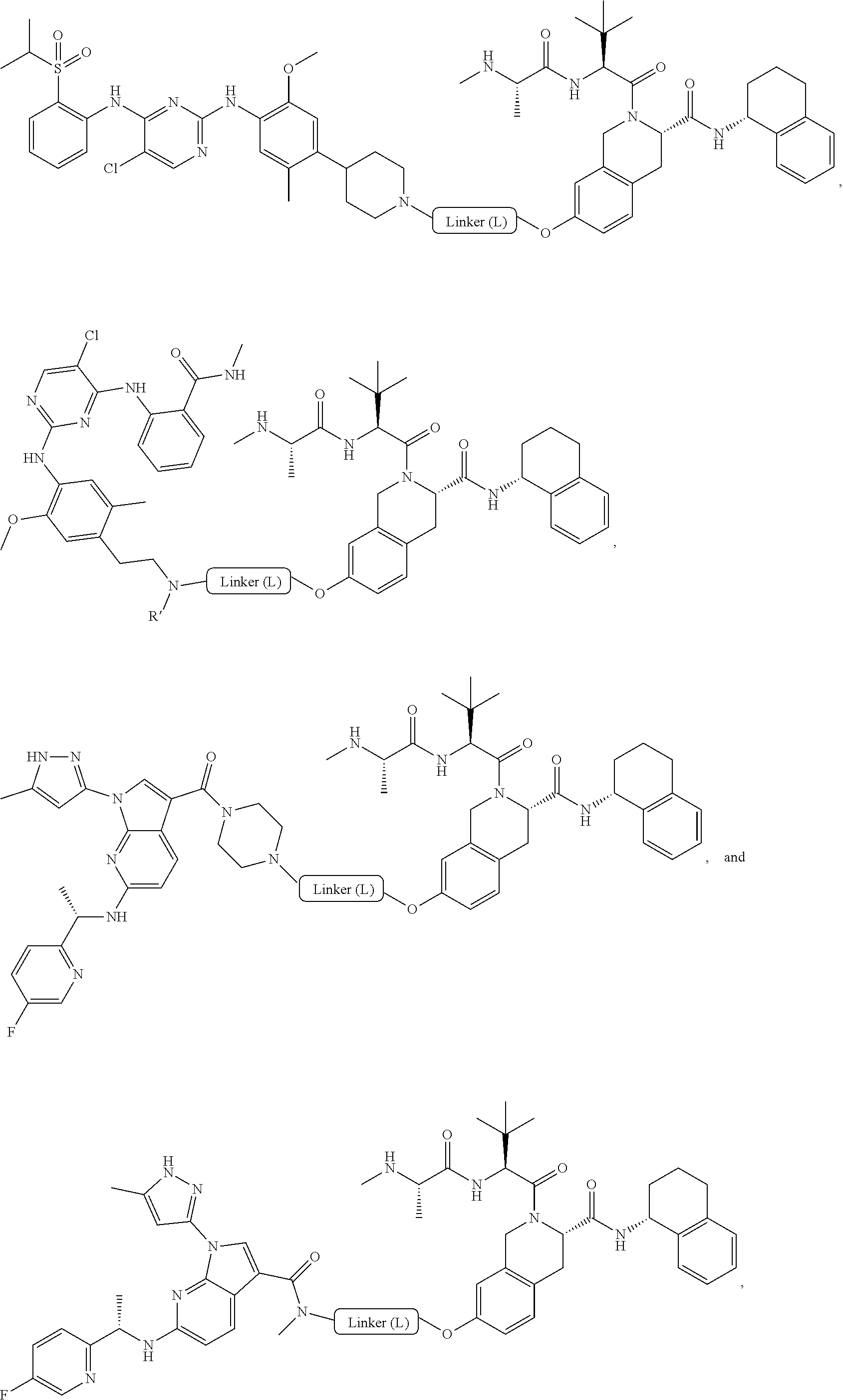

or a pharmaceutically acceptable salt or stereoisomer thereof.

Yet other degrons that bind IAPs and which may be suitable for use as degrons in the present invention are disclosed in International Patent Application Publications WO 2008/128171, WO 2008/016893, WO 2014/060768, and WO 2014/060767.

In some embodiments, the E3 ubiquitin ligase that is bound by the degron is murine double minute 2 (MDM2). Representative examples of degrons that bind MDM2 and may be suitable for use in the present invention are represented by any one of the following structures:

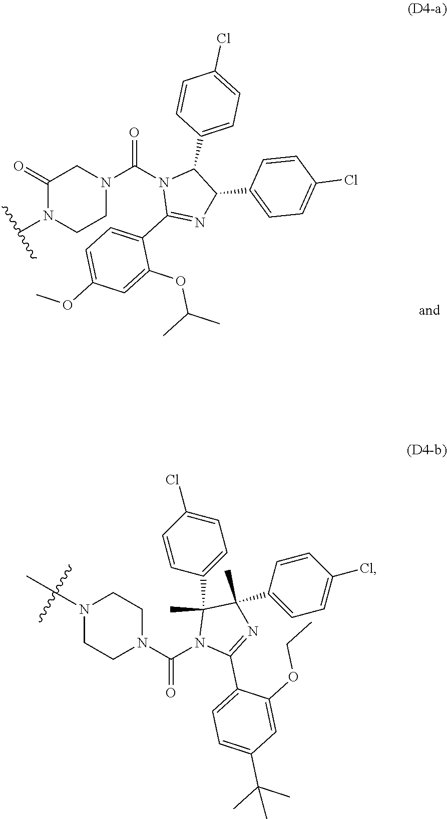 [SMILES: *N1CCN(C(=O)N2C(c3ccc(OC)cc3OC(C)C)=N[C@@H](c3ccc(Cl)cc3)[C@H]2c2ccc(Cl)cc2)CC1=O ; *N1CCN(C(=O)N2C(c3ccc(C(C)(C)C)cc3OCC)=N[C@@](C)(c3ccc(Cl)cc3)[C@@]2(C)c2ccc(Cl)cc2)CC1]

or a stereoisomer thereof.

Therefore, in some embodiments, bispecific compounds of the present invention are represented by any one of the following structures:

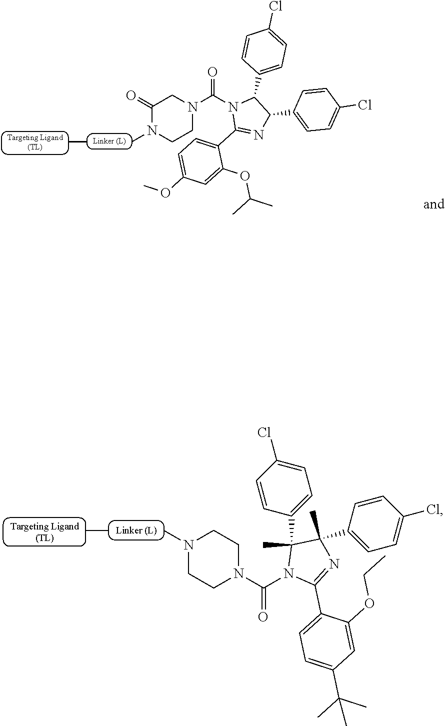

or a pharmaceutically acceptable salt or stereoisomer thereof.

In some embodiments, bispecific compounds of the present invention are represented by any one of the following structures:

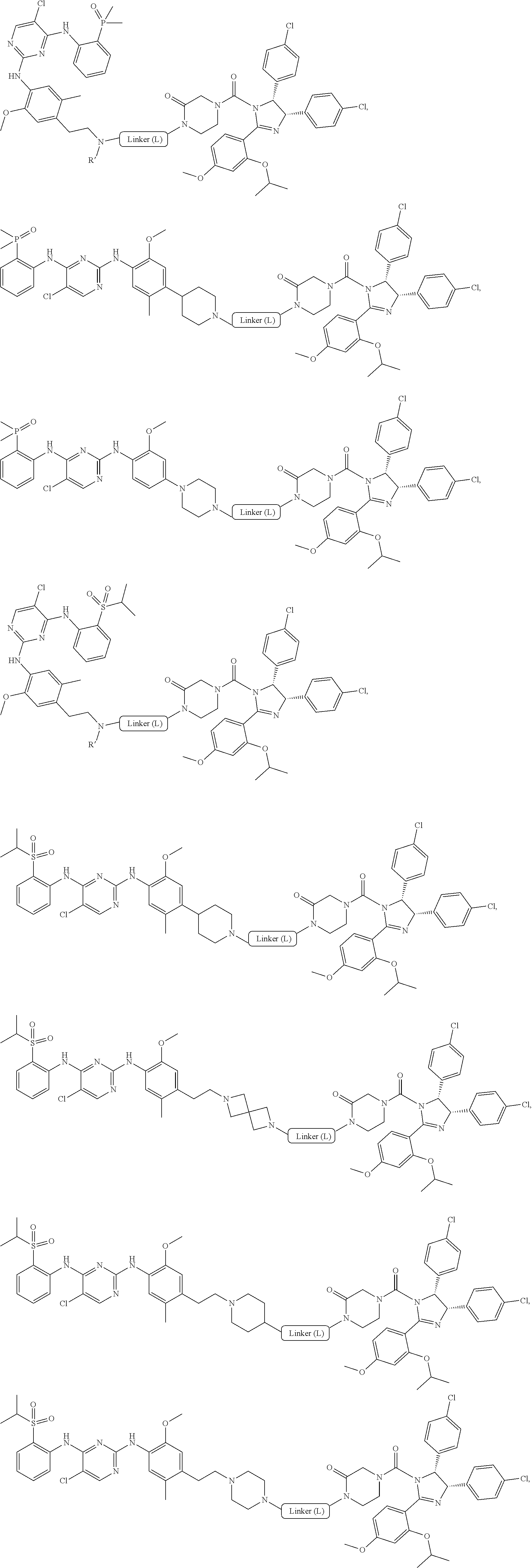

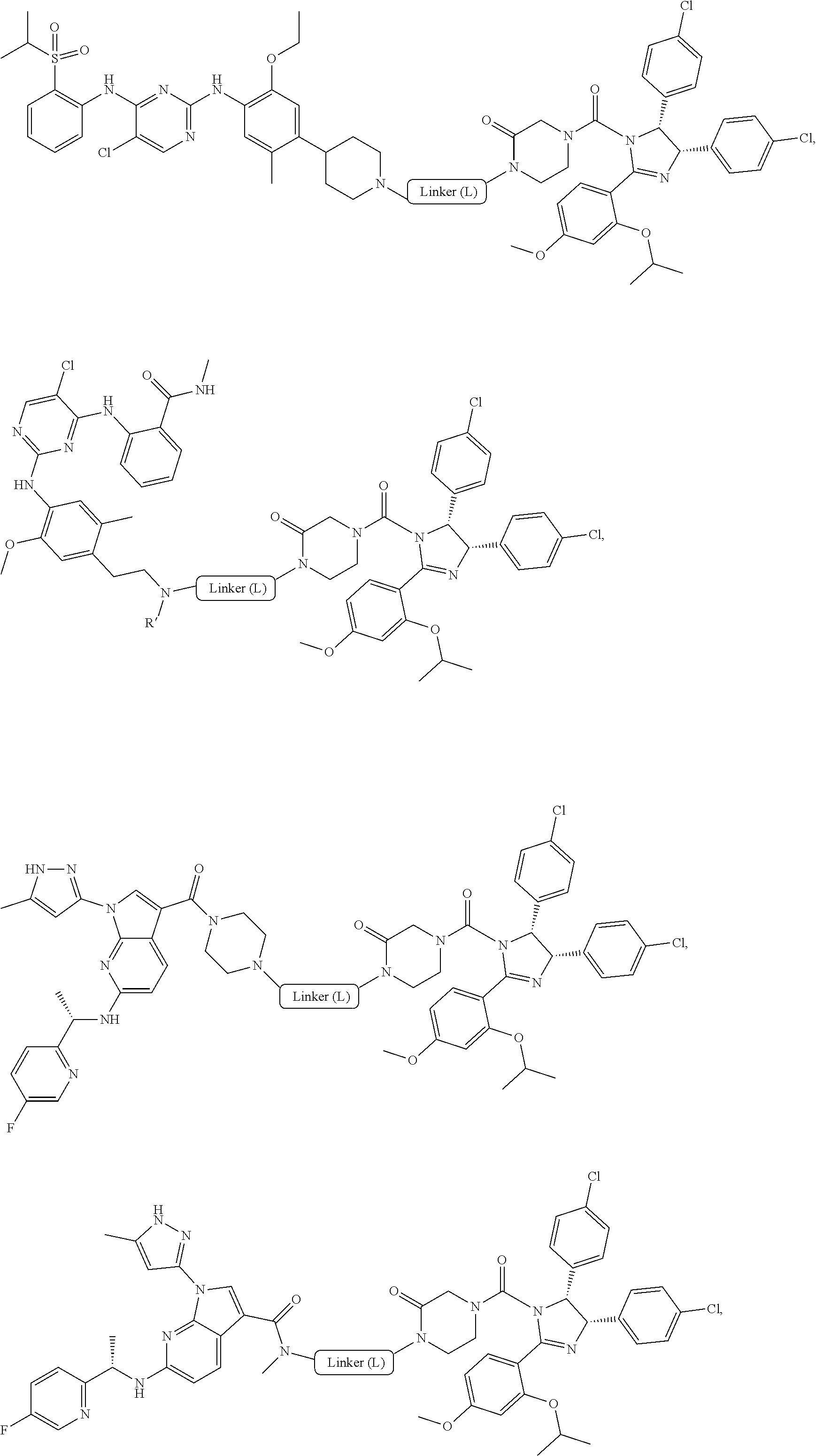

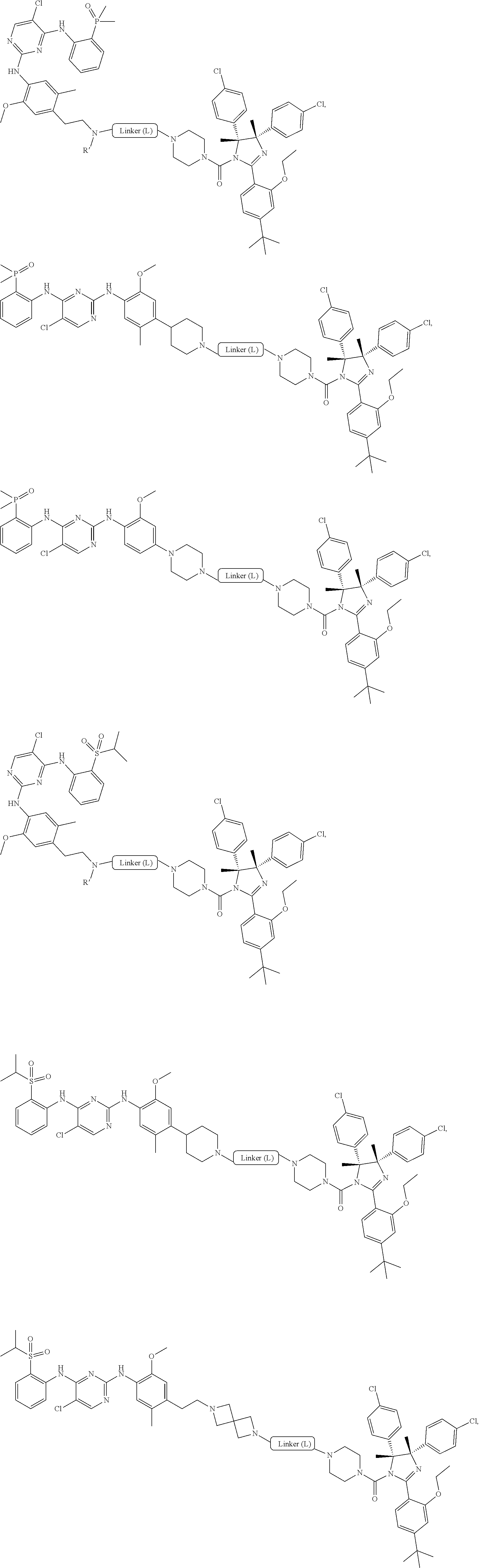

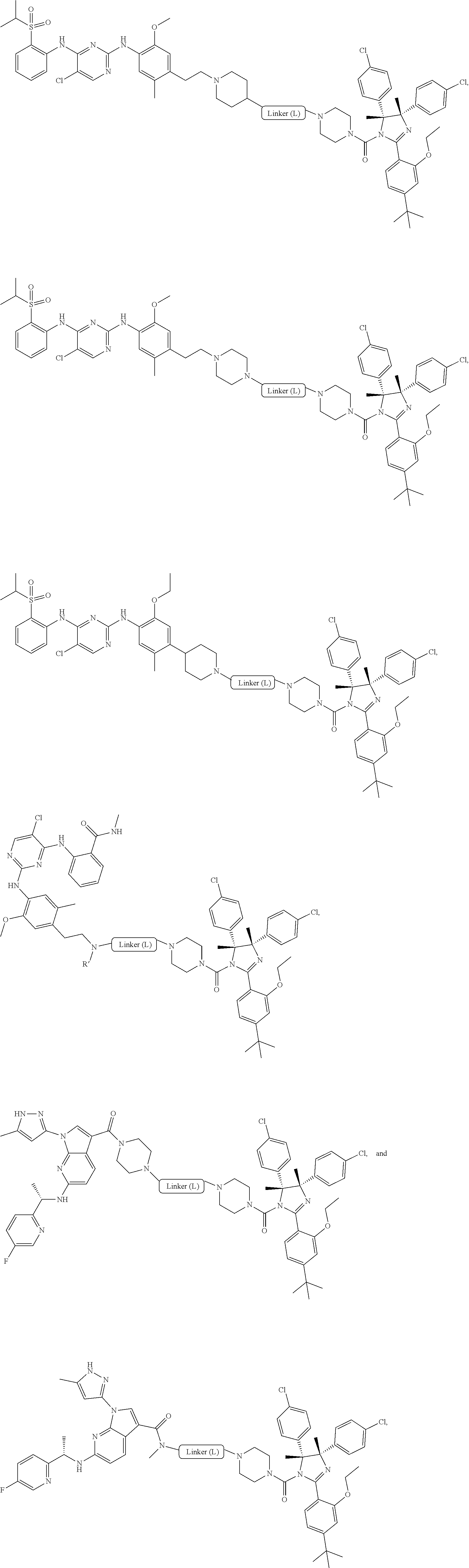

or a pharmaceutically acceptable salt or stereoisomer thereof.

Yet other degrons that bind MDM2 and which may be suitable for use as degrons in the present invention are disclosed in U.S. Pat. No. 9,993,472.

Therefore, in some embodiments, the bispecific compounds of this invention are represented by any structures generated by the combination of structures TL1 to TL3, L1 to L11, and the structures of the degrons described herein, including D1 to D4, or a pharmaceutically acceptable salt or stereoisomer thereof.

In some embodiments, the bispecific compounds of the present invention are represented by any one of the following structures:

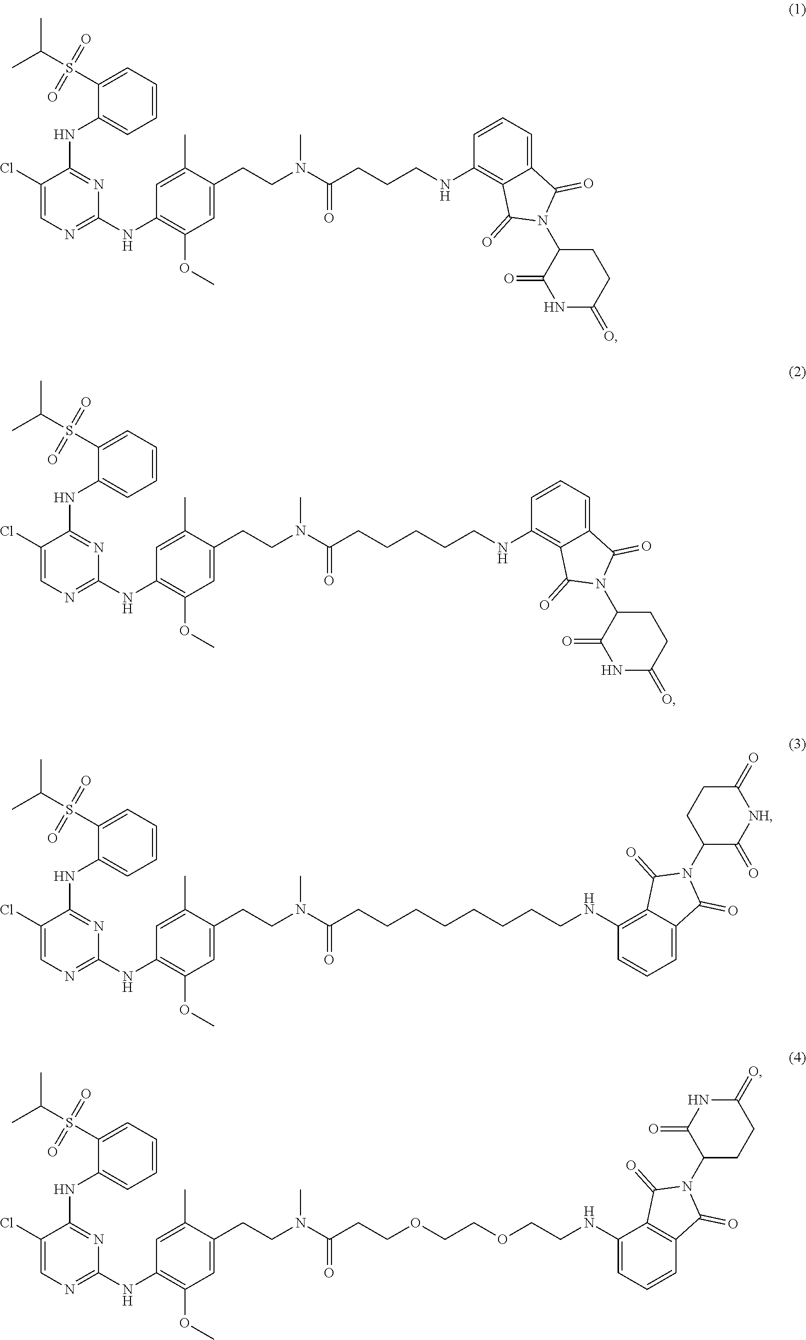

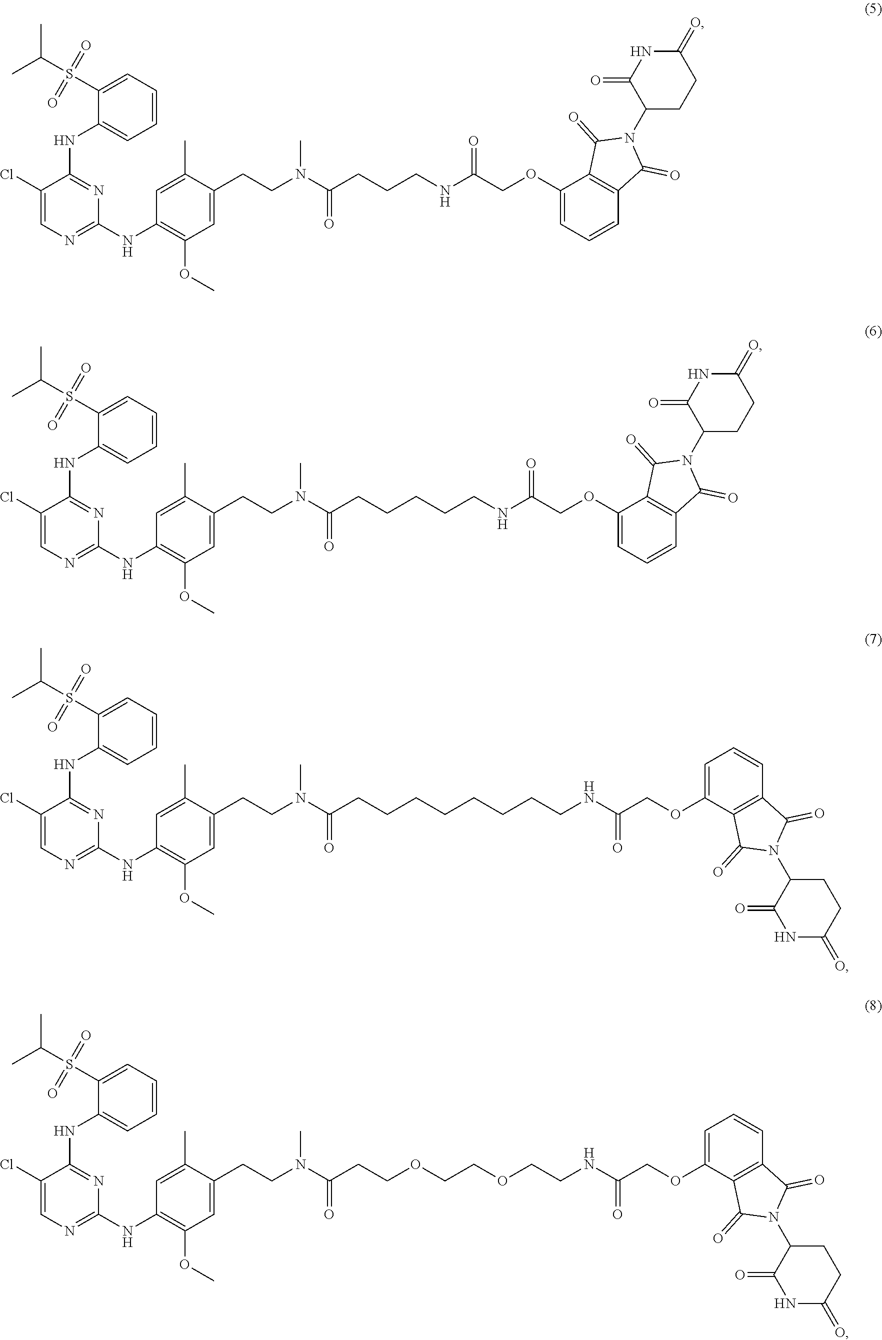 [SMILES: COc1cc(CCN(C)C(=O)CCCNC(=O)COc2cccc3c2C(=O)N(C2CCC(=O)NC2=O)C3=O)c(C)cc1Nc1ncc(Cl)c(Nc2ccccc2S(=O)(=O)C(C)C)n1 ; COc1cc(CCN(C)C(=O)CCCCCNC(=O)COc2cccc3c2C(=O)N(C2CCC(=O)NC2=O)C3=O)c(C)cc1Nc1ncc(Cl)c(Nc2ccccc2S(=O)(=O)C(C)C)n1 ; COc1cc(CCN(C)C(=O)CCCCCCCCNC(=O)COc2cccc3c2C(=O)N(C2CCC(=O)NC2=O)C3=O)c(C)cc1Nc1ncc(Cl)c(Nc2ccccc2S(=O)(=O)C(C)C)n1 ; COc1cc(CCN(C)C(=O)CCOCCOCCNC(=O)COc2cccc3c2C(=O)N(C2CCC(=O)NC2=O)C3=O)c(C)cc1Nc1ncc(Cl)c(Nc2ccccc2S(=O)(=O)C(C)C)n1]

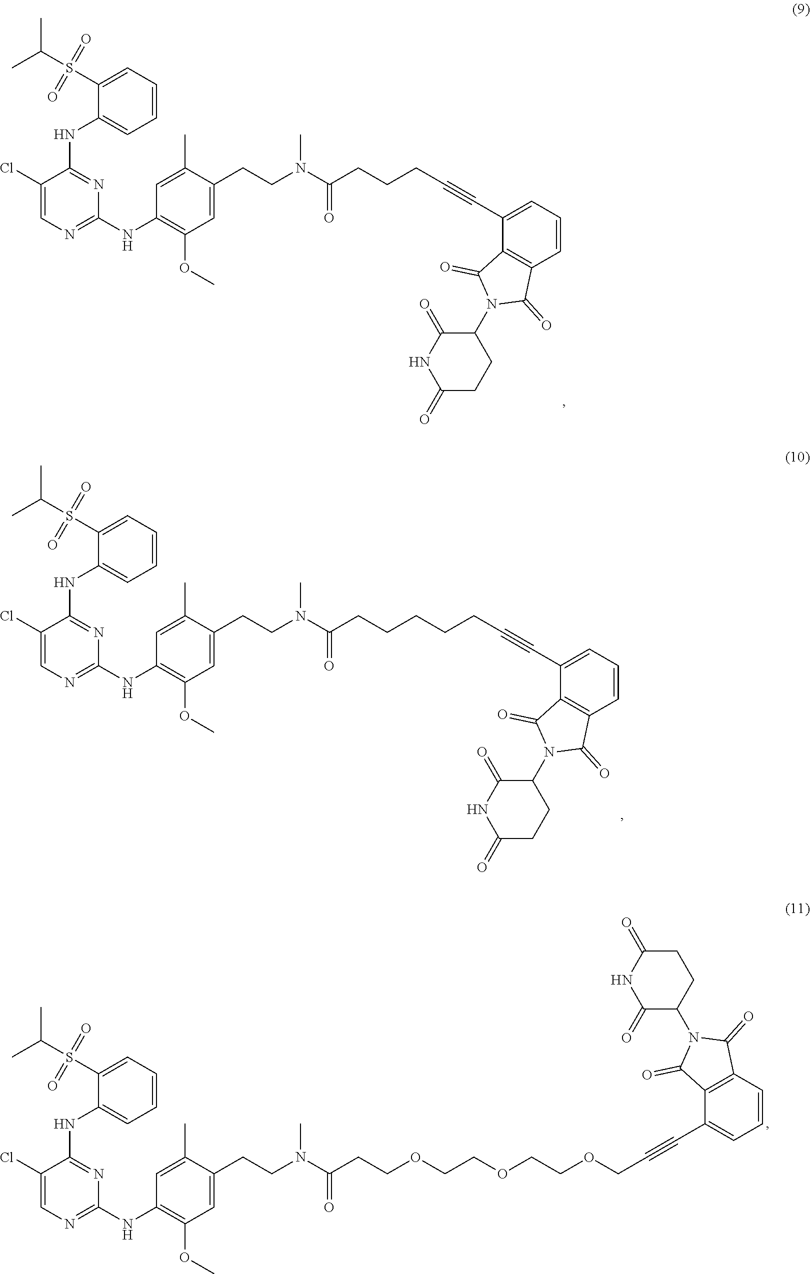 [SMILES: COc1cc(CCN(C)C(=O)CCCC#Cc2cccc3c2C(=O)N(C2CCC(=O)NC2=O)C3=O)c(C)cc1Nc1ncc(Cl)c(Nc2ccccc2S(=O)(=O)C(C)C)n1 ; COc1cc(CCN(C)C(=O)CCCCCC#Cc2cccc3c2C(=O)N(C2CCC(=O)NC2=O)C3=O)c(C)cc1Nc1ncc(Cl)c(Nc2ccccc2S(=O)(=O)C(C)C)n1 ; COc1cc(CCN(C)C(=O)CCOCCOCCOCC#Cc2cccc3c2C(=O)N(C2CCC(=O)NC2=O)C3=O)c(C)cc1Nc1ncc(Cl)c(Nc2ccccc2S(=O)(=O)C(C)C)n1]

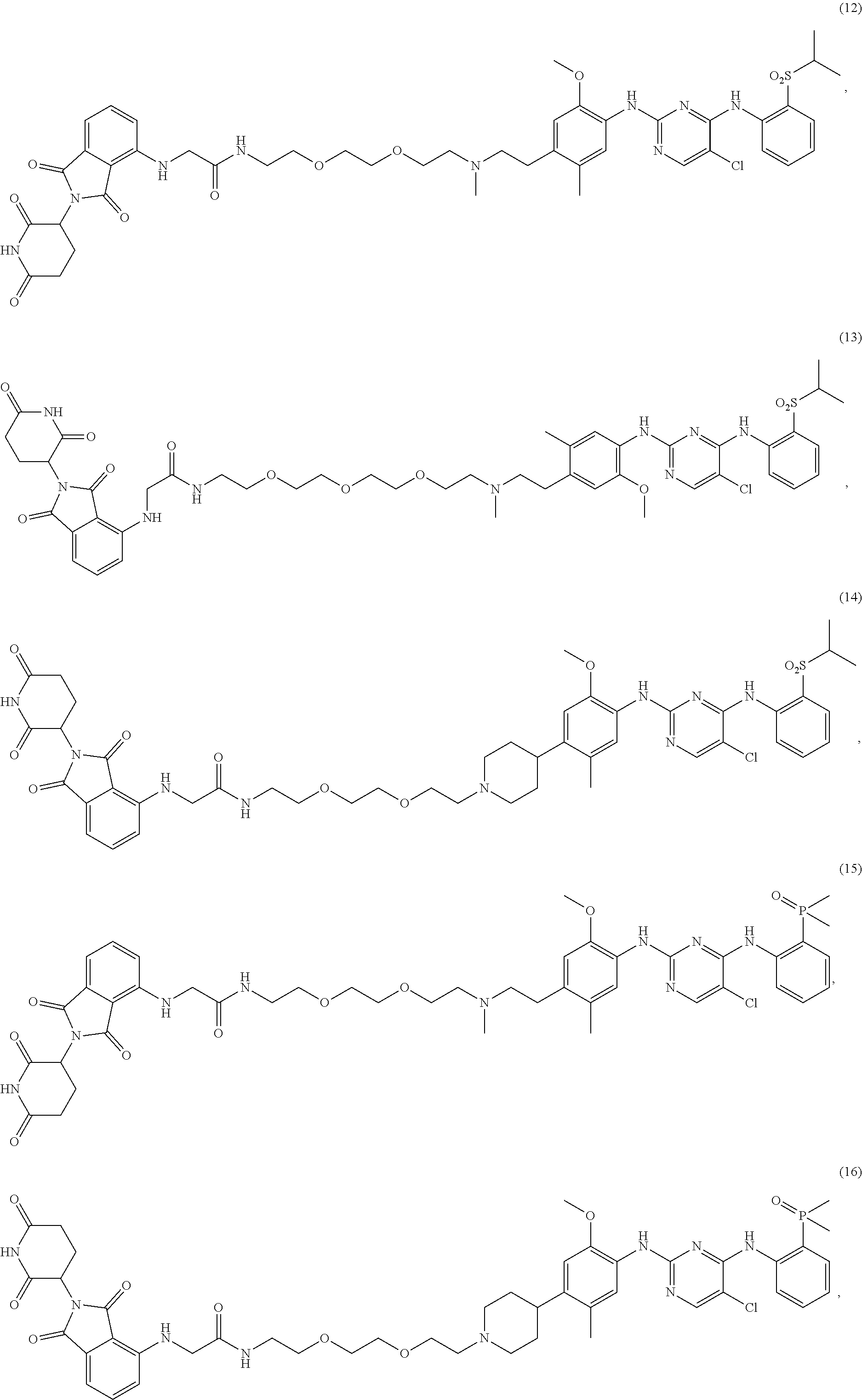

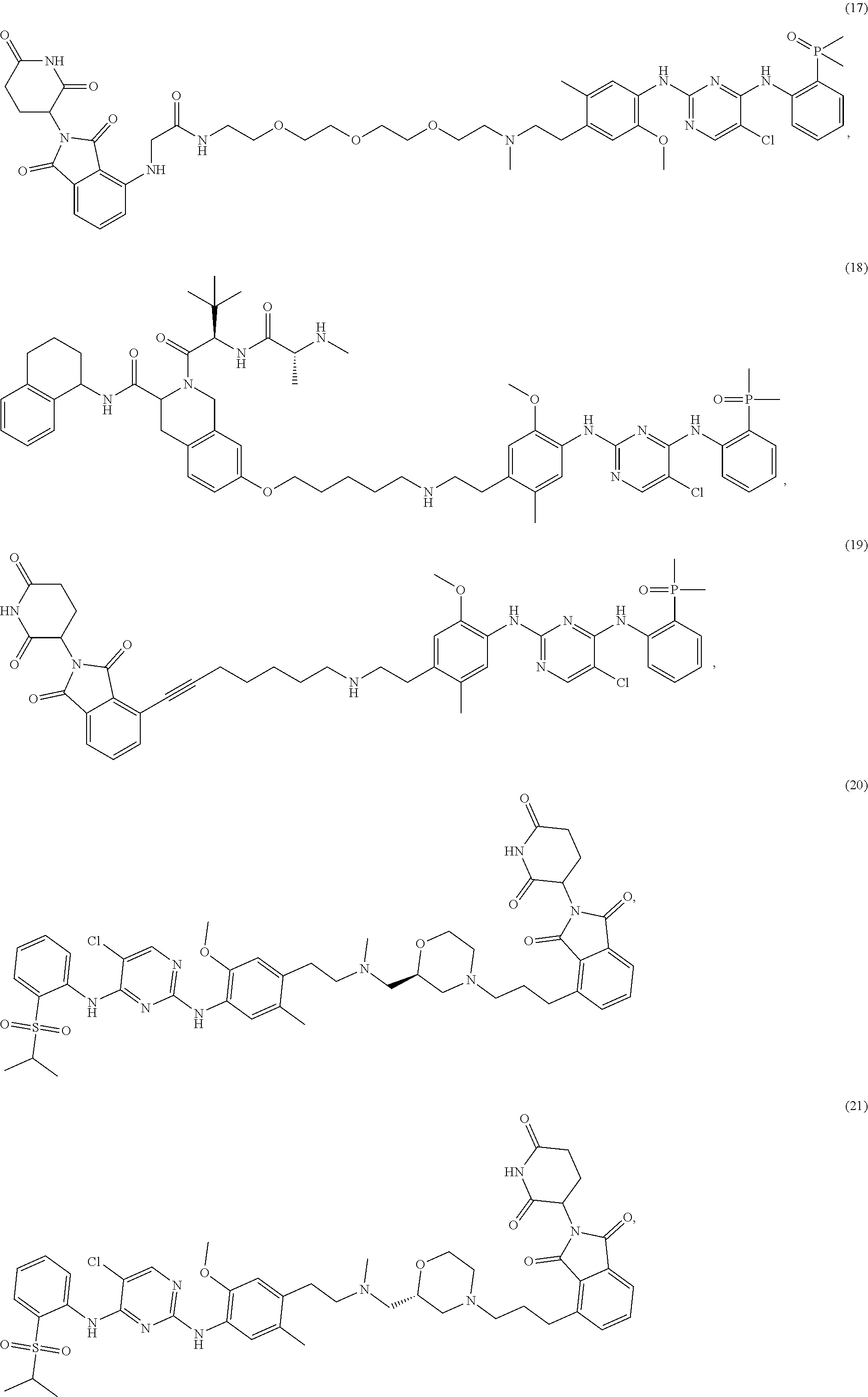

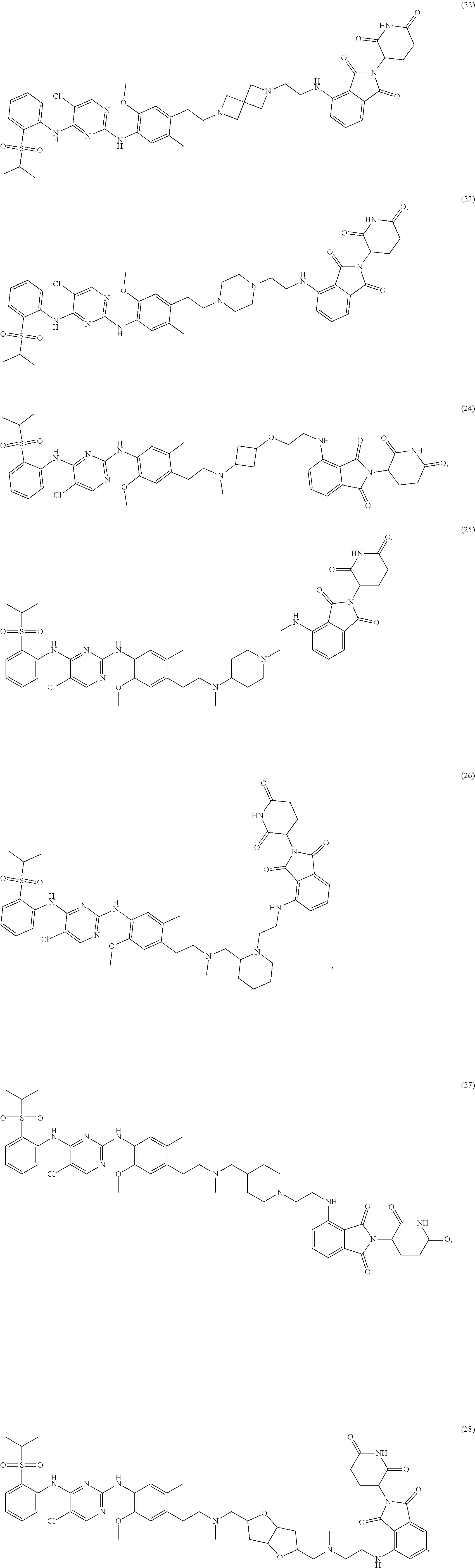

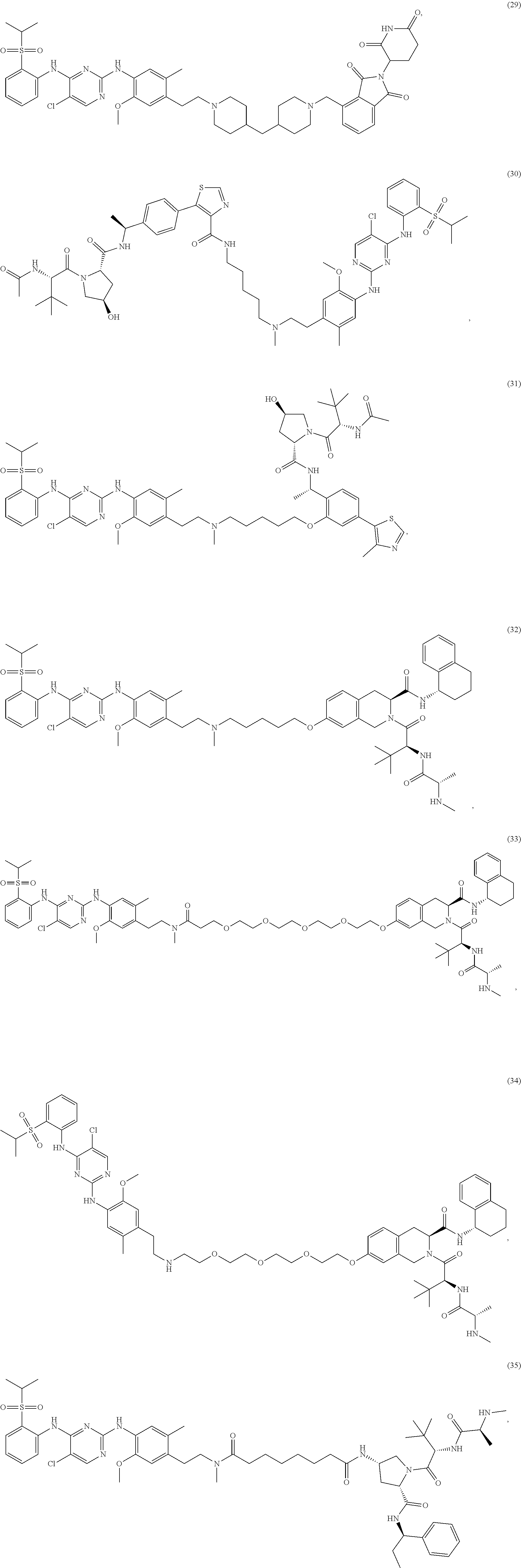

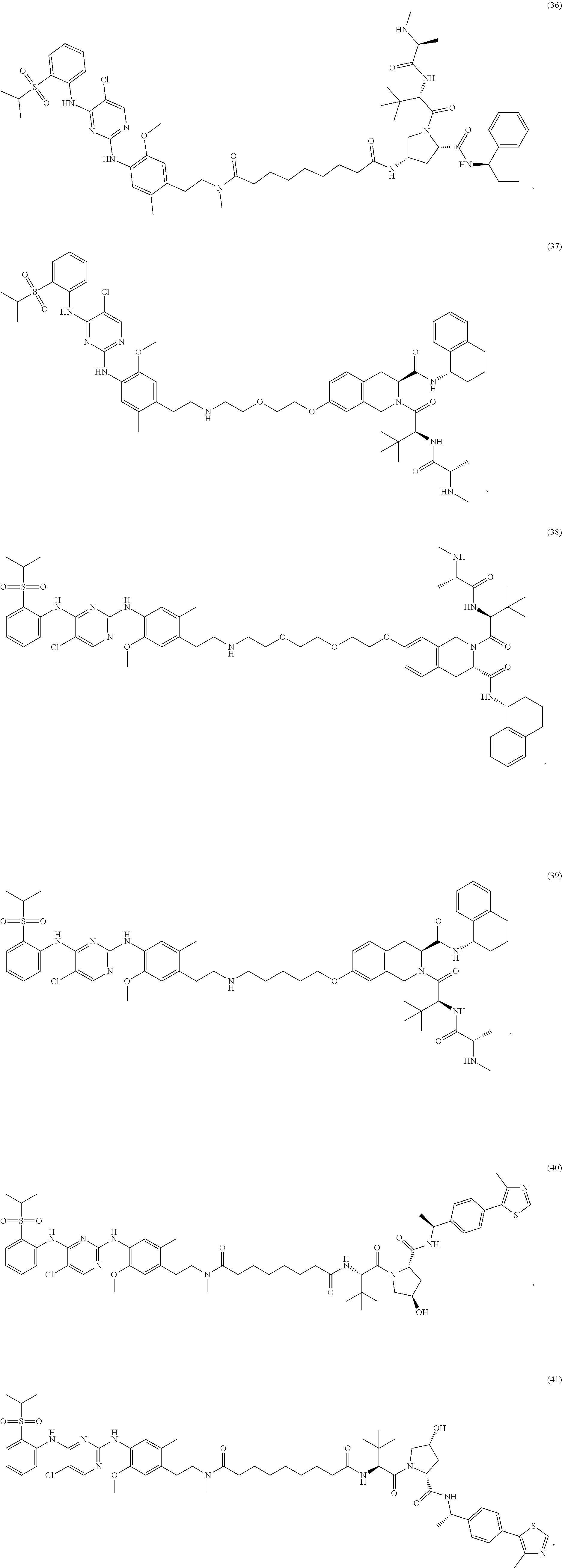

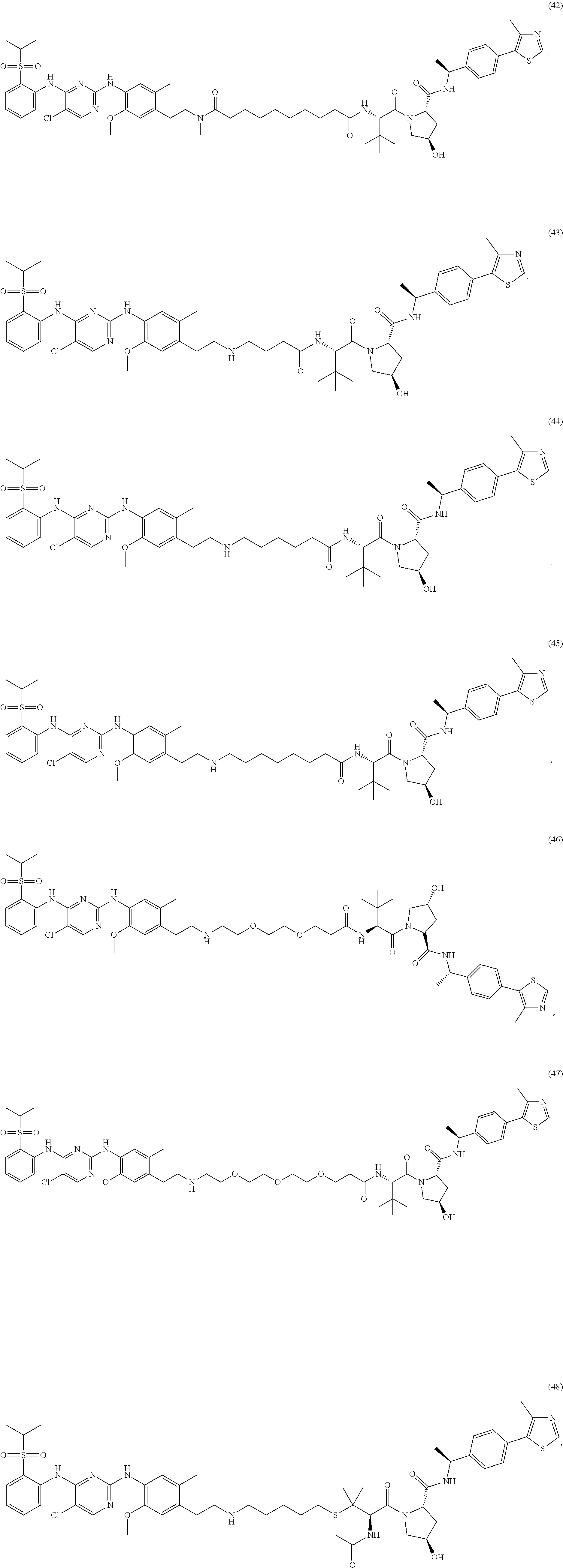

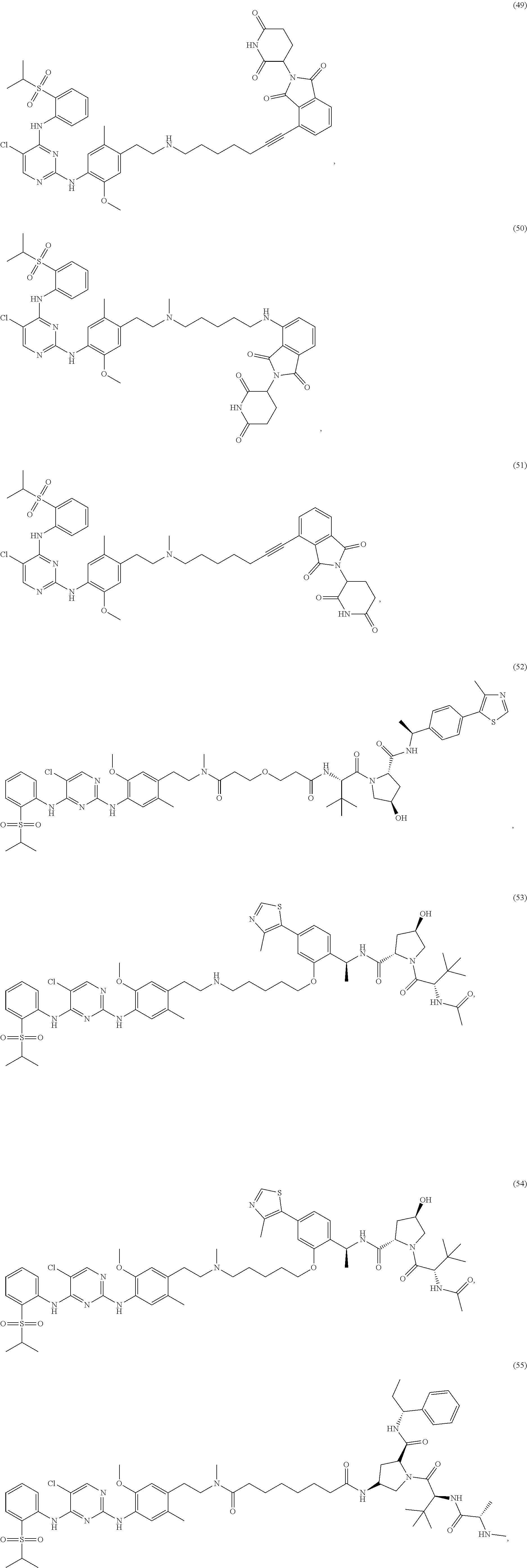

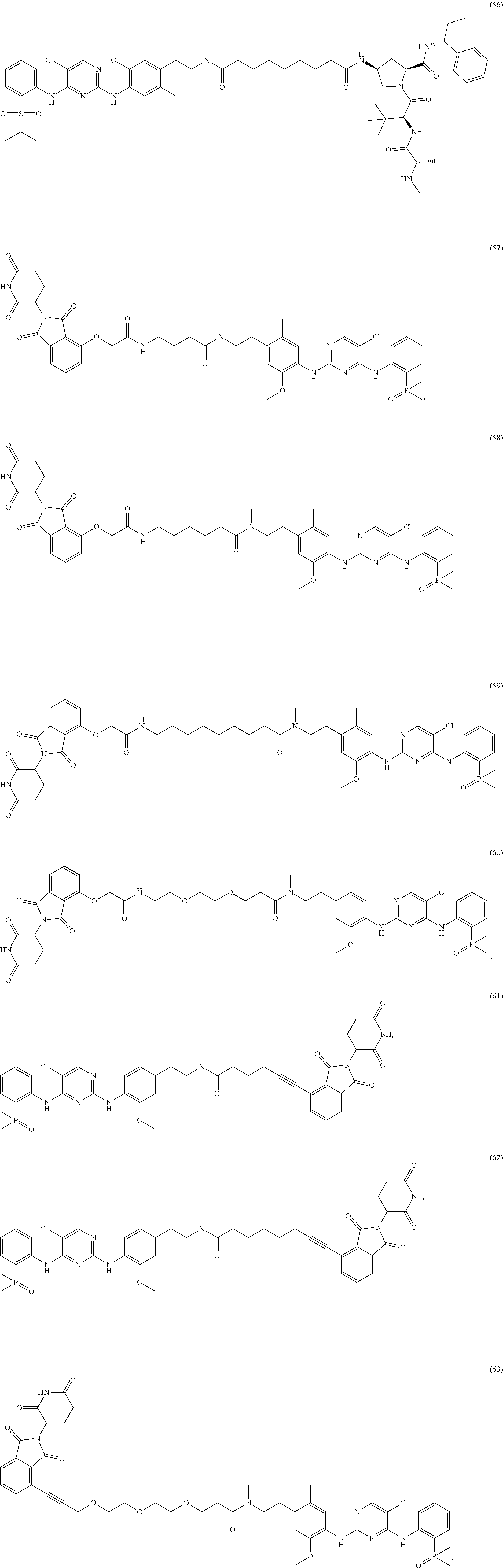

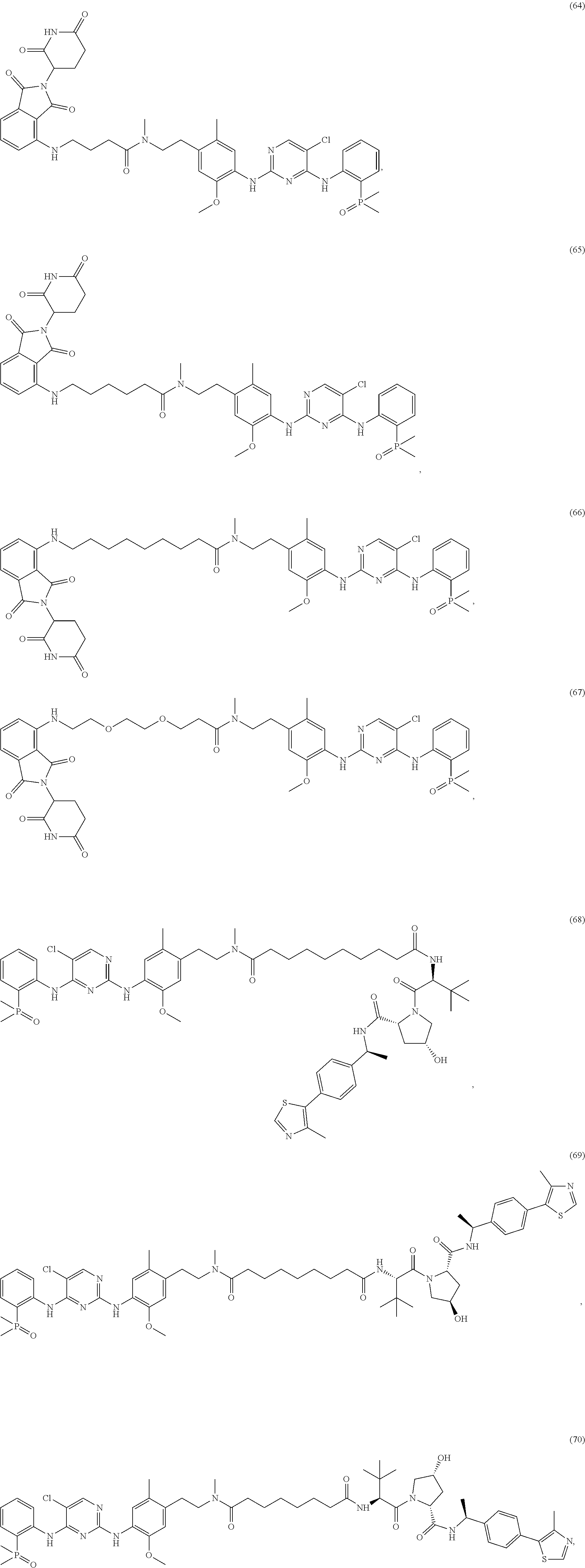

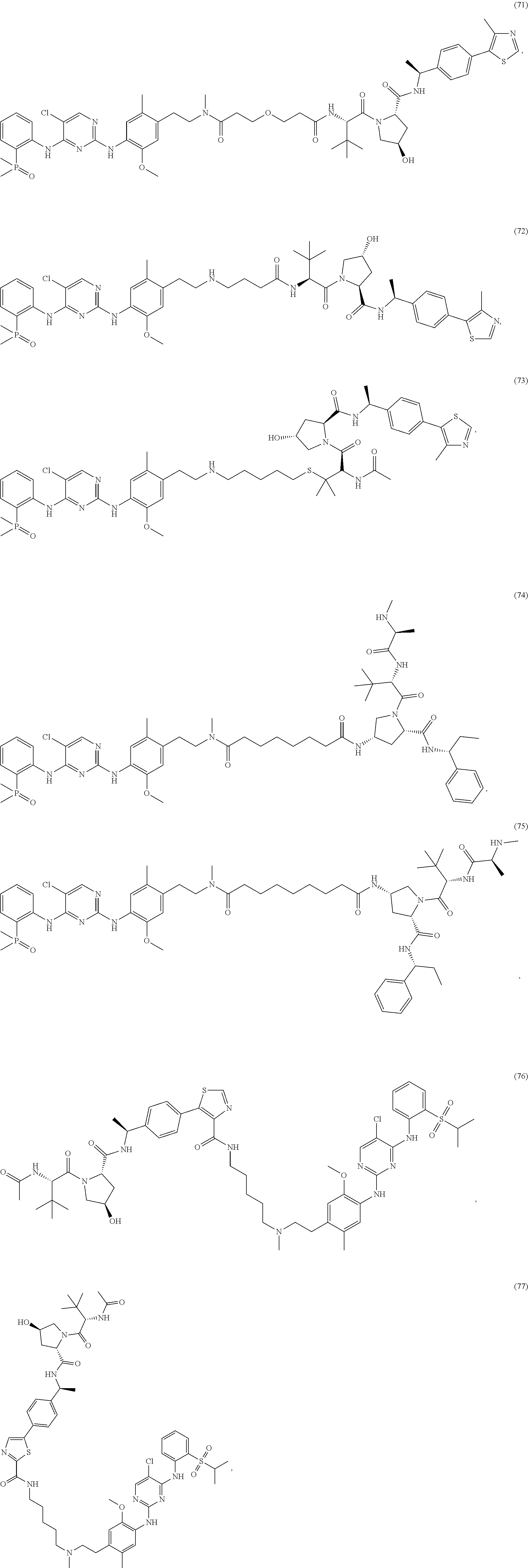

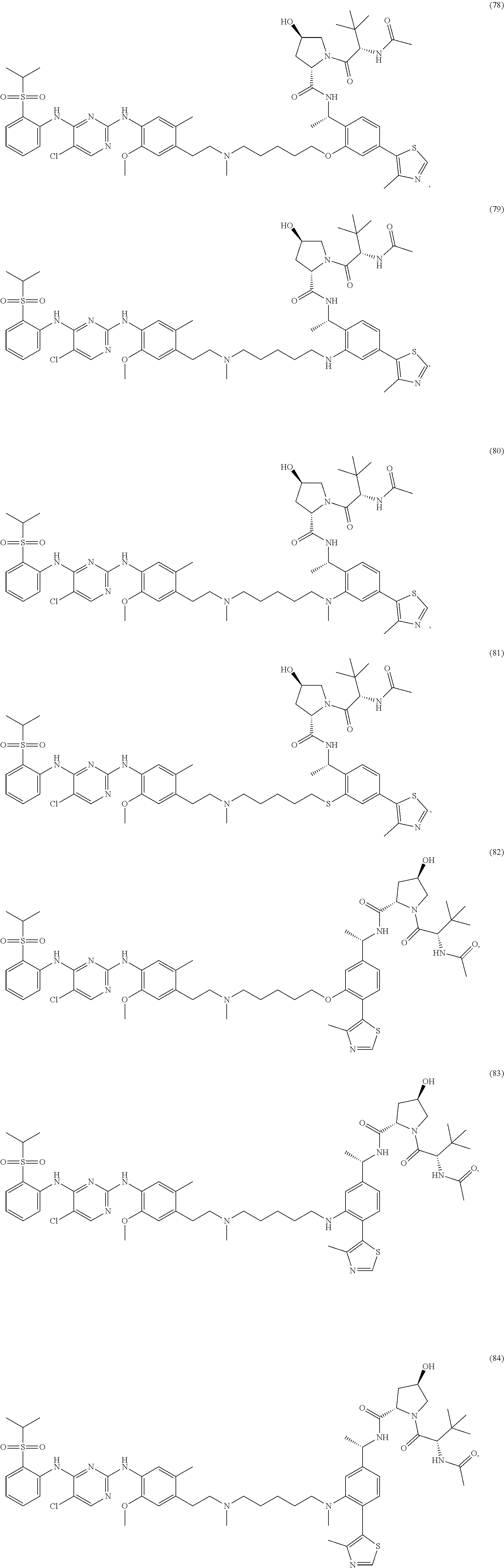

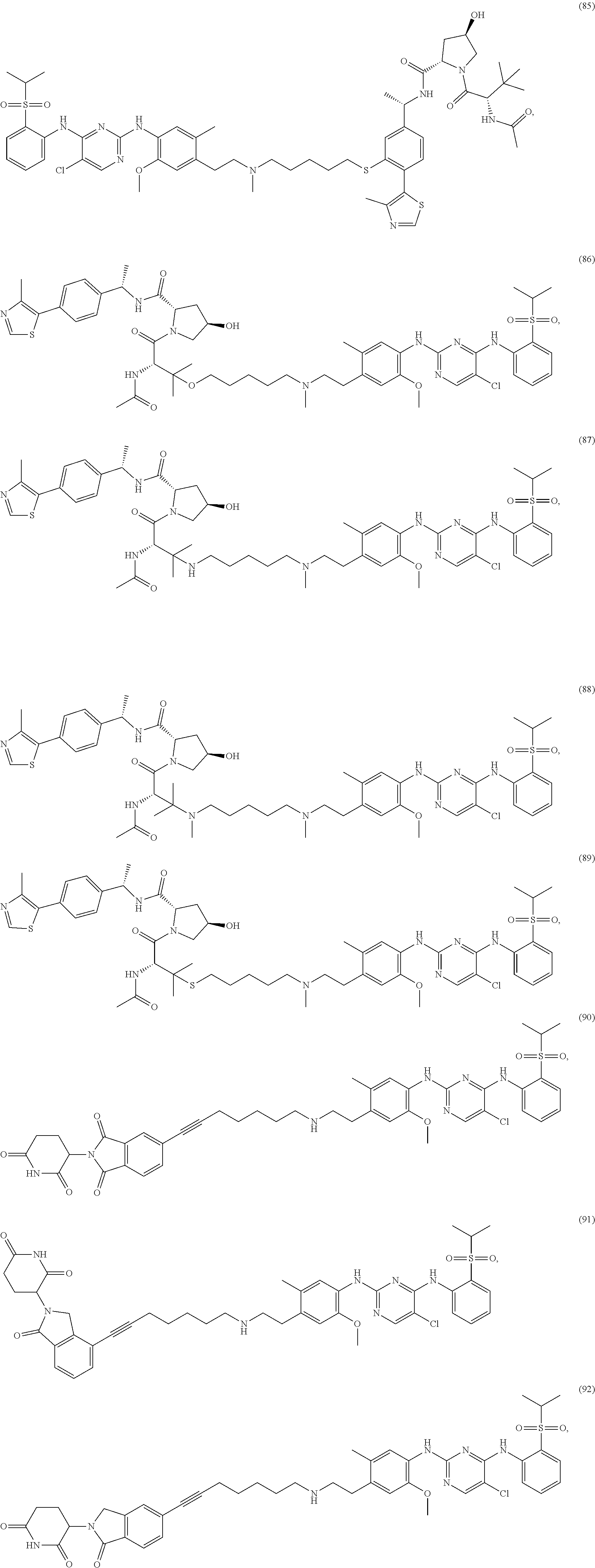

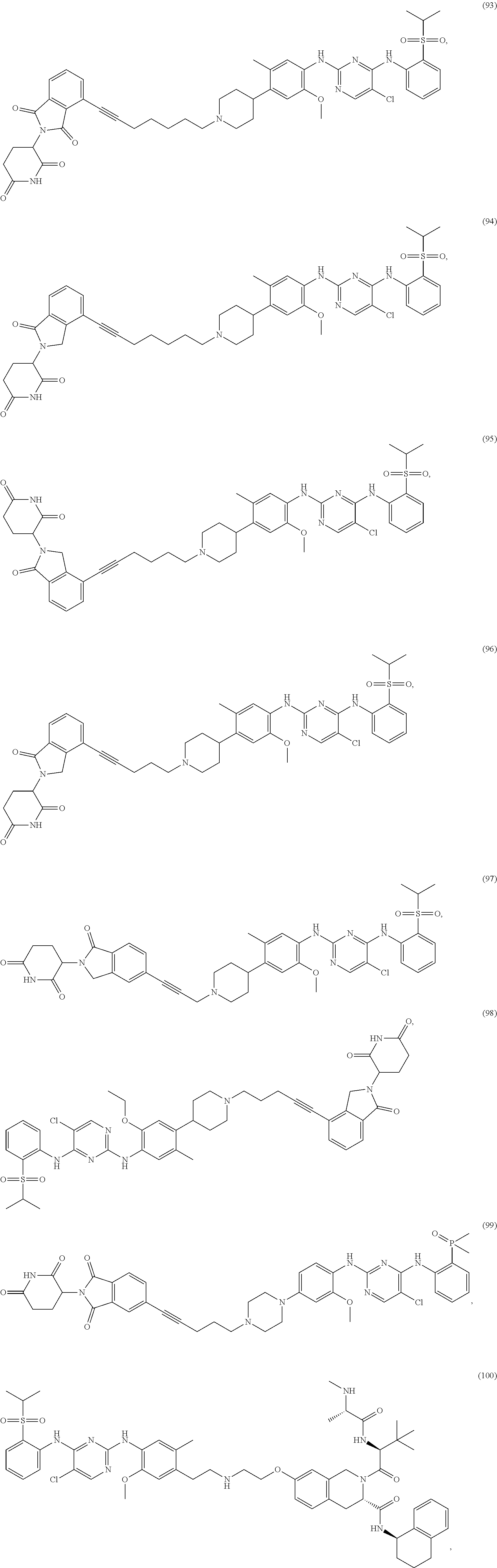

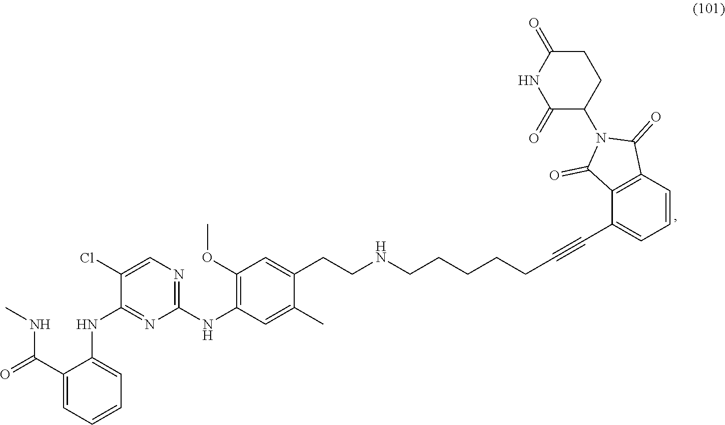 [SMILES: CNC(=O)c1ccccc1Nc1nc(Nc2cc(C)c(CCNCCCCCC#Cc3cccc4c3C(=O)N(C3CCC(=O)NC3=O)C4=O)cc2OC)ncc1Cl]

or a pharmaceutically acceptable salt or stereoisomer thereof.

Bispecific compounds of the present invention may be in the form of a free acid or free base, or a pharmaceutically acceptable salt. As used herein, the term “pharmaceutically acceptable” in the context of a salt refers to a salt of the compound that does not abrogate the biological activity or properties of the compound, and is relatively non-toxic, i.e., the compound in salt form may be administered to a subject without causing undesirable biological effects (such as dizziness or gastric upset) or interacting in a deleterious manner with any of the other components of the composition in which it is contained. The term “pharmaceutically acceptable salt” refers to a product obtained by reaction of the compound of the present invention with a suitable acid or a base. Examples of pharmaceutically acceptable salts of the compounds of this invention include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the invention can form pharmaceutically acceptable salts with various organic bases such as lysine, arginine, guanidine, diethanolamine or metformin.

Bispecific compounds of the invention may have at least one chiral center and therefore may be in the form of a stereoisomer, which as used herein, embraces all isomers of individual compounds that differ only in the orientation of their atoms in space. The term stereoisomer includes mirror image isomers (enantiomers which include the (R-) or (S-) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). The chiral centers of the compounds may undergo epimerization in vivo; therefore, for these compounds, administration of the compound in its (R-) form is considered equivalent to administration of the compound in its (S-) form. Accordingly, the compounds of the present invention may be made and used in the form of individual isomers and substantially free of other isomers, or in the form of a mixture of various isomers, e.g., racemic mixtures of stereoisomers.

The bispecific compounds of the invention embrace isotopic derivatives that have at least one desired isotopic substitution of an atom, at an amount above the natural abundance of the isotope, i.e., enriched. In one embodiment, the compound includes deuterium or multiple deuterium atoms. Substitution with heavier isotopes such as deuterium, i.e. ²H, may afford certain therapeutic advantages resulting from greater metabolic stability, for example, increased in vivo half-life or reduced dosage requirements, and therefore may be advantageous in some circumstances.

In addition to isotopic derivatives, the term “bispecific compounds of formula (I)” embraces N-oxides, crystalline forms (also known as polymorphs), active metabolites of the compounds having the same type of activity, tautomers, and unsolvated as well as solvated forms with pharmaceutically acceptable solvents such as water, ethanol, and the like, of the compounds.

### Methods of Synthesis

In another aspect, the present invention is directed to a method for making a bispecific compound of formula (I), or a pharmaceutically acceptable salt or stereoisomer thereof. Broadly, the inventive compounds or pharmaceutically-acceptable salts or stereoisomers thereof may be prepared by any process known to be applicable to the preparation of chemically related compounds. The compounds of the present invention will be better understood in connection with the synthetic schemes that described in various working examples and which illustrate non-limiting methods by which the compounds of the invention may be prepared.

### Pharmaceutical Compositions

Another aspect of the present invention is directed to a pharmaceutical composition that includes a therapeutically effective amount of a bispecific compound of formula (I) or a pharmaceutically acceptable salt or stereoisomer thereof, and a pharmaceutically acceptable carrier. The term “pharmaceutically acceptable carrier,” as known in the art, refers to a pharmaceutically acceptable material, composition or vehicle, suitable for administering compounds of the present invention to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may also include one or more pharmaceutically acceptable excipients.

Broadly, bispecific compounds of formula (I) and their pharmaceutically acceptable salts and stereoisomers may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). The type of formulation depends on the mode of administration which may include enteral (e.g., oral, buccal, sublingual and rectal), parenteral (e.g., subcutaneous (s.c.), intravenous (i.v.), intramuscular (i.m.), and intrasternal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal).

In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the bispecific compound may be administered relatively quickly such as in the case of a single-dose treatment and/or an acute condition.

In some embodiments, the bispecific compounds are formulated for oral or intravenous administration (e.g., systemic intravenous injection).

Accordingly, bispecific compounds of formula (I) may be formulated into solid compositions (e.g., powders, tablets, dispersible granules, capsules, cachets, and suppositories), liquid compositions (e.g., solutions in which the compound is dissolved, suspensions in which solid particles of the compound are dispersed, emulsions, and solutions containing liposomes, micelles, or nanoparticles, syrups and elixirs); semi-solid compositions (e.g., gels, suspensions and creams); and gases (e.g., propellants for aerosol compositions). Compounds may also be formulated for rapid, intermediate or extended release.

Solid dosage forms for oral administration include capsules, tablets, pills, powders, and granules. In such solid dosage forms, the active compound is mixed with a carrier such as sodium citrate or dicalcium phosphate and an additional carrier or excipient such as a) fillers or extenders such as starches, lactose, sucrose, glucose, mannitol, and silicic acid, b) binders such as, for example, methylcellulose, microcrystalline cellulose, hydroxypropylmethylcellulose, carboxymethylcellulose, sodium carboxymethylcellulose, alginates, gelatin, polyvinylpyrrolidinone, sucrose, and acacia, c) humectants such as glycerol, d) disintegrating agents such as crosslinked polymers (e.g., crosslinked polyvinylpyrrolidone (crospovidone), crosslinked sodium carboxymethyl cellulose (croscarmellose sodium), sodium starch glycolate, agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings. They may further contain an opacifying agent.

In some embodiments, bispecific compounds of formula (I) may be formulated in a hard or soft gelatin capsule. Representative excipients that may be used include pregelatinized starch, magnesium stearate, mannitol, sodium stearyl fumarate, lactose anhydrous, microcrystalline cellulose and croscarmellose sodium. Gelatin shells may include gelatin, titanium dioxide, iron oxides and colorants.

Liquid dosage forms for oral administration include solutions, suspensions, emulsions, micro-emulsions, syrups and elixirs. In addition to the compound, the liquid dosage forms may contain an aqueous or non-aqueous carrier (depending upon the solubility of the compounds) commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Oral compositions may also include an excipients such as wetting agents, suspending agents, coloring, sweetening, flavoring, and perfuming agents.

Injectable preparations for parenteral administration may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished by the use of a liquid suspension or crystalline or amorphous material with poor water solubility. Prolonged absorption of the compound from a parenterally administered formulation may also be accomplished by suspending the compound in an oily vehicle.

In certain embodiments, bispecific compounds of formula (I) may be administered in a local rather than systemic manner, for example, via injection of the conjugate directly into an organ, often in a depot preparation or sustained release formulation. In specific embodiments, long acting formulations are administered by implantation (for example subcutaneously or intramuscularly) or by intramuscular injection. Injectable depot forms are made by forming microencapsule matrices of the compound in a biodegradable polymer, e.g., polylactide-polyglycolides, poly(orthoesters) and poly(anhydrides). The rate of release of the compound may be controlled by varying the ratio of compound to polymer and the nature of the particular polymer employed. Depot injectable formulations are also prepared by entrapping the compound in liposomes or microemulsions that are compatible with body tissues. Furthermore, in other embodiments, the compound is delivered in a targeted drug delivery system, for example, in a liposome coated with organ-specific antibody. In such embodiments, the liposomes are targeted to and taken up selectively by the organ.

The compositions may be formulated for buccal or sublingual administration, examples of which include tablets, lozenges and gels.

The bispecific compounds of formula (I) may be formulated for administration by inhalation. Various forms suitable for administration by inhalation include aerosols, mists or powders. Pharmaceutical compositions may be delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In some embodiments, the dosage unit of a pressurized aerosol may be determined by providing a valve to deliver a metered amount. In some embodiments, capsules and cartridges including gelatin, for example, for use in an inhaler or insufflator, may be formulated containing a powder mix of the compound and a suitable powder base such as lactose or starch.

Bispecific compounds of formula (I) may be formulated for topical administration which as used herein, refers to administration intradermally by invention of the formulation to the epidermis. These types of compositions are typically in the form of ointments, pastes, creams, lotions, gels, solutions and sprays.

Representative examples of carriers useful in formulating bispecific compounds for topical application include solvents (e.g., alcohols, poly alcohols, water), creams, lotions, ointments, oils, plasters, liposomes, powders, emulsions, microemulsions, and buffered solutions (e.g., hypotonic or buffered saline). Creams, for example, may be formulated using saturated or unsaturated fatty acids such as stearic acid, palmitic acid, oleic acid, palmito-oleic acid, cetyl, or oleyl alcohols. Creams may also contain a non-ionic surfactant such as polyoxy-40-stearate.

In some embodiments, the topical formulations may also include an excipient, an example of which is a penetration enhancing agent. These agents are capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). Representative examples of penetration enhancing agents include triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Representative examples of yet other excipients that may be included in topical as well as in other types of formulations (to the extent they are compatible), include preservatives, antioxidants, moisturizers, emollients, buffering agents, solubilizing agents, skin protectants, and surfactants. Suitable preservatives include alcohols, quaternary amines, organic acids, parabens, and phenols. Suitable antioxidants include ascorbic acid and its esters, sodium bisulfite, butylated hydroxytoluene, butylated hydroxyanisole, tocopherols, and chelating agents like EDTA and citric acid. Suitable moisturizers include glycerin, sorbitol, polyethylene glycols, urea, and propylene glycol. Suitable buffering agents include citric, hydrochloric, and lactic acid buffers. Suitable solubilizing agents include quaternary ammonium chlorides, cyclodextrins, benzyl benzoate, lecithin, and polysorbates. Suitable skin protectants include vitamin E oil, allatoin, dimethicone, glycerin, petrolatum, and zinc oxide.

Transdermal formulations typically employ transdermal delivery devices and transdermal delivery patches wherein the compound is formulated in lipophilic emulsions or buffered, aqueous solutions, dissolved and/or dispersed in a polymer or an adhesive. Patches may be constructed for continuous, pulsatile, or on demand delivery of pharmaceutical agents. Transdermal delivery of the compounds may be accomplished by means of an iontophoretic patch. Transdermal patches may provide controlled delivery of the compounds wherein the rate of absorption is slowed by using rate-controlling membranes or by trapping the compound within a polymer matrix or gel. Absorption enhancers may be used to increase absorption, examples of which include absorbable pharmaceutically acceptable solvents that assist passage through the skin.

Ophthalmic formulations include eye drops.

Formulations for rectal administration include enemas, rectal gels, rectal foams, rectal aerosols, and retention enemas, which may contain conventional suppository bases such as cocoa butter or other glycerides, as well as synthetic polymers such as polyvinylpyrrolidone, PEG, and the like. Compositions for rectal or vaginal administration may also be formulated as suppositories which can be prepared by mixing the compound with suitable non-irritating carriers and excipients such as cocoa butter, mixtures of fatty acid glycerides, polyethylene glycol, suppository waxes, and combinations thereof, all of which are solid at ambient temperature but liquid at body temperature and therefore melt in the rectum or vaginal cavity and release the compound.

### Dosage Amounts

As used herein, the term, “therapeutically effective amount” refers to an amount of a bispecific compound of formula (I) or a pharmaceutically acceptable salt or a stereoisomer thereof that is effective in producing the desired therapeutic response in a particular patient suffering from a disease or disorder mediated by aberrant ALK or aberrant ALK and aberrant FAK activity. The term “therapeutically effective amount” therefore includes the amount of the bispecific compound or a pharmaceutically acceptable salt or a stereoisomer thereof, that when administered, induces a positive modification in the disease or disorder to be treated, or is sufficient to prevent development or progression of the disease or disorder, or alleviate to some extent, one or more of the symptoms of the disease or disorder being treated in a subject, or which simply kills or inhibits the growth of diseased (e.g., cancer) cells, or reduces the amounts of ALK or ALK and FAK in diseased cells.

The total daily dosage of the bispecific compounds and usage thereof may be decided in accordance with standard medical practice, e.g., by the attending physician using sound medical judgment. The specific therapeutically effective dose for any particular subject may depend upon a variety of factors including the disease or disorder being treated and the severity thereof (e.g., its present status); the age, body weight, general health, sex and diet of the subject; the time of administration, route of administration, and rate of excretion of the specific compound employed; the duration of the treatment; drugs used in combination or coincidental with the bispecific compound; and like factors well known in the medical arts (see, for example, Goodman and Gilman's, The Pharmacological Basis of Therapeutics, 10th Edition, A. Gilman, J. Hardman and L. Limbird, eds., McGraw-Hill Press, 155-173, 2001).

Bispecific compounds of formula (I) and their pharmaceutically acceptable salts and stereoisomers may be effective over a wide dosage range. In some embodiments, the total daily dosage (e.g., for adult humans) may range from about 0.001 to about 1600 mg, from 0.01 to about 1600 mg, from 0.01 to about 500 mg, from about 0.01 to about 100 mg, from about 0.5 to about 100 mg, from 1 to about 100-400 mg per day, from about 1 to about 50 mg per day, and from about 5 to about 40 mg per day, or in yet other embodiments from about 10 to about 30 mg per day. In some embodiments, the total daily dosage may range from 400 mg to 600 mg. Individual dosages may be formulated to contain the desired dosage amount depending upon the number of times the compound is administered per day. By way of example, capsules may be formulated with from about 1 to about 200 mg of compound (e.g., 1, 2, 2.5, 3, 4, 5, 10, 15, 20, 25, 50, 100, 150, and 200 mg). In some embodiments, the compound may be administered at a dose in range from about 0.01 mg to about 200 mg/kg of body weight per day. In some embodiments, a dose of from 0.1 to 100, e.g., from 1 to 30 mg/kg per day in one or more dosages per day may be effective. By way of example, a suitable dose for oral administration may be in the range of 1-30 mg/kg of body weight per day, and a suitable dose for intravenous administration may be in the range of 1-10 mg/kg of body weight per day.

In some embodiments, a bispecific compound is administered in a dose between 100 mg per day and 250 mg per day. In other embodiments the bispecific compound is administered in a dose between 200 mg per day and 400 mg per day, e.g., 250-350 mg per day.

### Methods of Use

In some aspects, the present invention is directed to treating diseases or disorders, cancerous and non-cancerous alike, characterized or mediated by aberrant (e.g., elevated levels of ALK or ALK and FAK or otherwise functionally abnormal e.g., deregulated ALK or deregulated ALK and FAK levels) ALK or aberrant ALK and aberrant FAK activity relative to a non-pathological state, which entails administering a therapeutically effective amount of a bispecific compound of formula (I) or a pharmaceutically acceptable salt or stereoisomer thereof, to a subject in need thereof. A “disease” is generally regarded as a state of health of a subject wherein the subject cannot maintain homeostasis, and wherein if the disease is not ameliorated then the subject's health continues to deteriorate. In contrast, a “disorder” (or “condition”) in a subject is a state of health in which the subject is able to maintain homeostasis, but in which the subject's state of health is less favorable than it would be in the absence of the disorder. Left untreated, a disorder may or may not cause a further decrease in the subject's state of health.

The term “subject” (or “patient”) as used herein includes all members of the animal kingdom prone to or suffering from the indicated disease or disorder. In some embodiments, the subject is a mammal, e.g., a human or a non-human mammal. The methods are also applicable to companion animals such as dogs and cats as well as livestock such as cows, horses, sheep, goats, pigs, and other domesticated and wild animals. A subject “in need of” treatment according to the present invention may be “suffering from or suspected of suffering from” a specific disease or disorder may have been positively diagnosed or otherwise presents with a sufficient number of risk factors or a sufficient number or combination of signs or symptoms such that a medical professional could diagnose or suspect that the subject was suffering from the disease or disorder. Therefore, subjects suffering from, and suspected of suffering from, a specific disease or disorder are not necessarily two distinct groups.

In some embodiments, the inventive bispecific compounds may be useful in the treatment of cell proliferative diseases and disorders (e.g., cancer or benign neoplasms). As used herein, the term “cell proliferative disease or disorder” refers to the conditions characterized by aberrant cell growth, or both, including noncancerous conditions such as neoplasms, precancerous conditions, benign tumors, and cancer.

Exemplary types of non-cancerous (e.g., cell proliferative) diseases or disorders that may be amenable to treatment with bispecific compounds of the present invention include inflammatory diseases and conditions, autoimmune diseases, neurodegenerative diseases, heart diseases, viral diseases, chronic and acute kidney diseases or injuries, metabolic diseases, allergic disorders, and genetic diseases.

Representative examples of specific non-cancerous diseases and disorders include rheumatoid arthritis, alopecia areata, lymphoproliferative conditions, autoimmune hematological disorders (e.g., hemolytic anemia, aplastic anemia, anhidrotic ectodermal dysplasia, pure red cell anemia and idiopathic thrombocytopenia), cholecystitis, acromegaly, rheumatoid spondylitis, osteoarthritis, gout, scleroderma, sepsis, septic shock, dacryoadenitis, cryopyrin associated periodic syndrome (CAPS), endotoxic shock, endometritis, gram-negative sepsis, keratoconjunctivitis sicca, toxic shock syndrome, asthma, adult respiratory distress syndrome, chronic obstructive pulmonary disease, chronic pulmonary inflammation, chronic graft rejection, hidradenitis suppurativa, inflammatory bowel disease, Crohn's disease, Behcet's syndrome, systemic lupus erythematosus, glomerulonephritis, multiple sclerosis, juvenile-onset diabetes, autoimmune uveoretinitis, autoimmune vasculitis, thyroiditis, Addison's disease, lichen planus, appendicitis, bullous pemphigus, pemphigus vulgaris, pemphigus foliaceus, paraneoplastic pemphigus, myasthenia gravis, immunoglobulin A nephropathy, Hashimoto's disease, Sjogren's syndrome, vitiligo, Wegener granulomatosis, granulomatous orchitis, autoimmune oophoritis, sarcoidosis, rheumatic carditis, ankylosing spondylitis, Grave's disease, autoimmune thrombocytopenic purpura, psoriasis, psoriatic arthritis, eczema, dermatitis herpetiformis, ulcerative colitis, pancreatic fibrosis, hepatitis, hepatic fibrosis, CD14 mediated sepsis, non-CD14 mediated sepsis, acute and chronic renal disease, irritable bowel syndrome, pyresis, restenosis, cervicitis, stroke and ischemic injury, neural trauma, acute and chronic pain, allergic rhinitis, allergic conjunctivitis, chronic heart failure, congestive heart failure, acute coronary syndrome, cachexia, malaria, leprosy, leishmaniasis, Lyme disease, Reiter's syndrome, acute synovitis, muscle degeneration, bursitis, tendonitis, tenosynovitis, herniated, ruptured, or prolapsed intervertebral disk syndrome, osteopetrosis, rhinosinusitis, thrombosis, silicosis, pulmonary sarcosis, bone resorption diseases, such as osteoporosis, fibromyalgia, AIDS and other viral diseases such as Herpes Zoster, Herpes Simplex I or II, influenza virus and cytomegalovirus, diabetes Type I and II, obesity, insulin resistance and diabetic retinopathy, 22q11.2 deletion syndrome, Angelman syndrome, Canavan disease, celiac disease, Charcot-Marie-Tooth disease, color blindness, Cri du chat, Down syndrome, cystic fibrosis, Duchenne muscular dystrophy, haemophilia, Klinefleter's syndrome, neurofibromatosis, phenylketonuria, Prader-Willi syndrome, sickle cell disease, Tay-Sachs disease, Turner syndrome, urea cycle disorders, thalassemia, otitis, pancreatitis, parotitis, pericarditis, peritonitis, pharyngitis, pleuritis, phlebitis, pneumonitis, uveitis, polymyositis, proctitis, interstitial lung fibrosis, dermatomyositis, atherosclerosis, arteriosclerosis, amyotrophic lateral sclerosis, asociality, varicosis, vaginitis, depression, and Sudden Infant Death Syndrome.

In some embodiments, the bispecific compounds may be useful in the treatment of non-cancerous neurodegenerative diseases and disorders. As used herein, the term “neurodegenerative diseases and disorders” refers to the conditions characterized by progressive degeneration or death of nerve cells, or both, including problems with movement (ataxias), or mental functioning (dementias). Representative examples of such diseases and disorders include Alzheimer's disease (AD) and AD-related dementias, Parkinson's disease (PD) and PD-related dementias, prion disease, motor neuron diseases (MND), Huntington's disease (HD), Pick's syndrome, spinocerebellar ataxia (SCA), spinal muscular atrophy (SMA), primary progressive aphasia (PPA), amyotrophic lateral sclerosis (ALS), traumatic brain injury (TBI), multiple sclerosis (MS), dementias (e.g., vascular dementia (VaD), Lewy body dementia (LBD), semantic dementia, and frontotemporal lobar dementia (FTD).

In some embodiments, the bispecific compounds may be useful in the treatment of autoimmune diseases and disorders. As used herein, the term “autoimmune disease” refers to conditions where the immune system produces antibodies that attack normal body tissues. Representative examples of such diseases include Sjogren's syndrome, Hashimoto thyroiditis, rheumatoid arthritis, juvenile (type 1) diabetes, polymyositis, scleroderma, Addison disease, lupus (e.g., systemic lupus erythematosus), vitiligo, pernicious anemia, glomerulonephritis, pulmonary fibrosis, celiac disease, polymyalgia rheumatica, multiple sclerosis, ankylosing spondylitis, alopecia areata, vasculitis, and temporal arteritis.

In some embodiments, the methods are directed to treating subjects having cancer. In some embodiments, the cancer is an ALK-positive cancer. In some embodiments, the cancer is an ALK-negative cancer. Broadly, the bispecific compounds of the present invention may be effective in the treatment of carcinomas (solid tumors including both primary and metastatic tumors), sarcomas, melanomas, and hematological cancers (cancers affecting blood including lymphocytes, bone marrow and/or lymph nodes) such as leukemia, lymphoma and multiple myeloma. Adult tumors/cancers and pediatric tumors/cancers are included. The cancers may be vascularized, or not yet substantially vascularized, or non-vascularized tumors.

Representative examples of cancers includes adrenocortical carcinoma, AIDS-related cancers (e.g., Kaposi's and AIDS-related lymphoma), appendix cancer, childhood cancers (e.g., childhood cerebellar astrocytoma, childhood cerebral astrocytoma), basal cell carcinoma, skin cancer (non-melanoma), biliary cancer, extrahepatic bile duct cancer, intrahepatic bile duct cancer, bladder cancer, urinary bladder cancer, brain cancer (e.g., gliomas and glioblastomas such as brain stem glioma, gestational trophoblastic tumor glioma, cerebellar astrocytoma, cerebral astrocytoma/malignant glioma, ependymoma, medulloblastoma, supratentorial primitive neuroectodeimal tumors, visual pathway and hypothalamic glioma), breast cancer, bronchial adenomas/carcinoids, carcinoid tumor, nervous system cancer (e.g., central nervous system cancer, central nervous system lymphoma), cervical cancer, chronic myeloproliferative disorders, colorectal cancer (e.g., colon cancer, rectal cancer), lymphoid neoplasm, mycosis fungoids, Sezary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastrointestinal cancer (e.g., stomach cancer, small intestine cancer, gastrointestinal carcinoid tumor, gastrointestinal stromal tumor (GIST)), cholangiocarcinoma, germ cell tumor, ovarian germ cell tumor, head and neck cancer, neuroendocrine tumors, Hodgkin's lymphoma, Ann Arbor stage III and stage IV childhood Non-Hodgkin's lymphoma, ROS1-positive refractory Non-Hodgkin's lymphoma, leukemia, lymphoma, multiple myeloma, hypopharyngeal cancer, intraocular melanoma, ocular cancer, islet cell tumors (endocrine pancreas), renal cancer (e.g., Wilm's Tumor, renal cell carcinoma), liver cancer, lung cancer (e.g., non-small cell lung cancer and small cell lung cancer), ALK-positive anaplastic large cell lymphoma, ALK-positive advanced malignant solid neoplasm, Waldenstrom's macroglobulinema, melanoma, intraocular (eye) melanoma, merkel cell carcinoma, mesothelioma, metastatic squamous neck cancer with occult primary, multiple endocrine neoplasia (MEN), myelodysplastic syndromes, myelodysplastic/myeloproliferative diseases, nasopharyngeal cancer, neuroblastoma, oral cancer (e.g., mouth cancer, lip cancer, oral cavity cancer, tongue cancer, oropharyngeal cancer, throat cancer, laryngeal cancer), ovarian cancer (e.g., ovarian epithelial cancer, ovarian germ cell tumor, ovarian low malignant potential tumor), pancreatic cancer, islet cell pancreatic cancer, paranasal sinus and nasal cavity cancer, parathyroid cancer, penile cancer, pharyngeal cancer, pheochromocytoma, pineoblastoma, metastatic anaplastic thyroid cancer, undifferentiated thyroid cancer, papillary thyroid cancer, pituitary tumor, plasma cell neoplasm/multiple myeloma, pleuropulmonary blastoma, prostate cancer, retinoblastoma, rhabdomyosarcoma, salivary gland cancer, uterine cancer (e.g., endometrial uterine cancer, uterine sarcoma, uterine corpus cancer), squamous cell carcinoma, testicular cancer, thymoma, thymic carcinoma, thyroid cancer, juvenile xanthogranuloma, transitional cell cancer of the renal pelvis and ureter and other urinary organs, urethral cancer, gestational trophoblastic tumor, vaginal cancer, vulvar cancer, hepatoblastoma, rhabdoid tumor, and Wilms tumor.

In some embodiments, the cancer is anaplastic large cell lymphoma (ALCL), inflammatory myofibroblastic tumor (IMT), breast cancer, colorectal cancer, esophageal squamous cell cancer (ESCC), large B-cell lymphoma (DLBCL), renal cell cancer (RCC), or non-small cell lung cancer (NSCLC).

Sarcomas that may be treatable with compounds of the present invention include both soft tissue and bone cancers alike, representative examples of which include osteosarcoma or osteogenic sarcoma (bone) (e.g., Ewing's sarcoma), chondrosarcoma (cartilage), leiomyosarcoma (smooth muscle), rhabdomyosarcoma (skeletal muscle), mesothelial sarcoma or mesothelioma (membranous lining of body cavities), fibrosarcoma (fibrous tissue), angiosarcoma or hemangioendothelioma (blood vessels), liposarcoma (adipose tissue), glioma or astrocytoma (neurogenic connective tissue found in the brain), myxosarcoma (primitive embryonic connective tissue) and mesenchymous or mixed mesodermal tumor (mixed connective tissue types), and histiocytic sarcoma (immune cancer).

In some embodiments, methods of the present invention entail treatment of subjects having cell proliferative diseases or disorders of the hematological system, liver, brain, lung, colon, pancreas, prostate, ovary, breast, skin, and endometrium.

As used herein, “cell proliferative diseases or disorders of the hematological system” include lymphoma, leukemia, myeloid neoplasms, mast cell neoplasms, myelodysplasia, benign monoclonal gammopathy, lymphomatoid papulosis, polycythemia vera, chronic myelocytic leukemia, agnogenic myeloid metaplasia, and essential thrombocythemia. Representative examples of hematologic cancers may therefore include leukemia, multiple myeloma, and lymphoma (including T-cell lymphoma, Hodgkin's lymphoma, non-Hodgkin's lymphoma (NHL). Examples of NHL include diffuse large B-cell lymphoma (DLBCL), follicular lymphoma (FL), mantle cell lymphoma (MCL), cutaneous T-cell lymphoma (CTCL) (including mycosis fungoides and Sezary syndrome), peripheral T-cell lymphoma (PTCL) (including anaplastic large-cell lymphoma (ALCL), angioimmunoblastic T-cell lymphoma, hepatosplenic T-cell lymphoma, epithelial T-cell lymphoma, and gamma-delta T-cell lymphoma), germinal center B-cell-like diffuse large B-cell lymphoma, activated B-cell-like diffuse large B-cell lymphoma, Burkitt's lymphoma/leukemia, mantle cell lymphoma, mediastinal (thymic) large B-cell lymphoma, follicular lymphoma, marginal zone lymphoma, lymphoplasmacytic lymphoma/Waldenstrom macroglobulinemia, refractory NHL, relapsed NHL, childhood lymphomas, and small lymphocytic lymphoma. Examples of leukemia include childhood leukemia, hairy-cell leukemia, acute lymphocytic leukemia, acute myelocytic leukemia, acute myeloid leukemia (e.g., acute monocytic leukemia), chronic lymphocytic leukemia, chronic myelocytic leukemia, chronic myelogenous leukemia, mast cell leukemia, myeloid neoplasms and mast cell neoplasms.

As used herein, “cell proliferative diseases or disorders of the liver” include all forms of cell proliferative disorders affecting the liver. Cell proliferative disorders of the liver may include liver cancer (e.g., hepatocellular carcinoma, intrahepatic cholangiocarcinoma and hepatoblastoma), a precancer or precancerous condition of the liver, benign growths or lesions of the liver, and malignant growths or lesions of the liver, and metastatic lesions in tissue and organs in the body other than the liver. Cell proliferative disorders of the liver may include hyperplasia, metaplasia, and dysplasia of the liver.

As used herein, “cell proliferative diseases or disorders of the brain” include all forms of cell proliferative disorders affecting the brain. Cell proliferative disorders of the brain may include brain cancer (e.g., gliomas, glioblastomas, meningiomas, pituitary adenomas, vestibular schwannomas, and primitive neuroectodermal tumors (medulloblastomas)), a precancer or precancerous condition of the brain, benign growths or lesions of the brain, and malignant growths or lesions of the brain, and metastatic lesions in tissue and organs in the body other than the brain. Cell proliferative disorders of the brain may include hyperplasia, metaplasia, and dysplasia of the brain.

As used herein, “cell proliferative diseases or disorders of the lung” include all forms of cell proliferative disorders affecting lung cells. Cell proliferative disorders of the lung include lung cancer, precancer and precancerous conditions of the lung, benign growths or lesions of the lung, hyperplasia, metaplasia, and dysplasia of the lung, and metastatic lesions in the tissue and organs in the body other than the lung. Lung cancer includes all forms of cancer of the lung, e.g., malignant lung neoplasms, carcinoma in situ, typical carcinoid tumors, and atypical carcinoid tumors. Lung cancer includes small cell lung cancer (“SLCL”), non-small cell lung cancer (“NSCLC”), squamous cell carcinoma, adenocarcinoma, small cell carcinoma, large cell carcinoma, squamous cell carcinoma, and mesothelioma. Lung cancer can include “scar carcinoma”, bronchioveolar carcinoma, giant cell carcinoma, spindle cell carcinoma, and large cell neuroendocrine carcinoma. Lung cancer also includes lung neoplasms having histologic and ultrastructural heterogeneity (e.g., mixed cell types). In some embodiments, a compound of the present invention may be used to treat non-metastatic or metastatic lung cancer (e.g., NSCLC, ALK-positive NSCLC, NSCLC harboring ROS1 Rearrangement, Lung Adenocarcinoma, and Squamous Cell Lung Carcinoma).

As used herein, “cell proliferative diseases or disorders of the colon” include all forms of cell proliferative disorders affecting colon cells, including colon cancer, a precancer or precancerous conditions of the colon, adenomatous polyps of the colon and metachronous lesions of the colon. Colon cancer includes sporadic and hereditary colon cancer, malignant colon neoplasms, carcinoma in situ, typical carcinoid tumors, and atypical carcinoid tumors, adenocarcinoma, squamous cell carcinoma, and squamous cell carcinoma. Colon cancer can be associated with a hereditary syndrome such as hereditary nonpolyposis colorectal cancer, familiar adenomatous polyposis, MYH associated polyposis, Gardner's syndrome, Peutz-Jeghers syndrome, Turcot's syndrome and juvenile polyposis. Cell proliferative disorders of the colon may also be characterized by hyperplasia, metaplasia, or dysplasia of the colon.

As used herein, “cell proliferative diseases or disorders of the pancreas” include all forms of cell proliferative disorders affecting pancreatic cells. Cell proliferative disorders of the pancreas may include pancreatic cancer, a precancer or precancerous condition of the pancreas, hyperplasia of the pancreas, dysplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas, including ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma, and pancreatic neoplasms having histologic and ultrastructural heterogeneity (e.g., mixed cell).

As used herein, “cell proliferative diseases or disorders of the prostate” include all forms of cell proliferative disorders affecting the prostate. Cell proliferative disorders of the prostate may include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate may include hyperplasia, metaplasia, and dysplasia of the prostate.

As used herein, “cell proliferative diseases or disorders of the ovary” include all forms of cell proliferative disorders affecting cells of the ovary. Cell proliferative disorders of the ovary may include a precancer or precancerous condition of the ovary, benign growths or lesions of the ovary, ovarian cancer, and metastatic lesions in tissue and organs in the body other than the ovary. Cell proliferative disorders of the ovary may include hyperplasia, metaplasia, and dysplasia of the ovary.

As used herein, “cell proliferative diseases or disorders of the breast” include all forms of cell proliferative disorders affecting breast cells. Cell proliferative disorders of the breast may include breast cancer, a precancer or precancerous condition of the breast, benign growths or lesions of the breast, and metastatic lesions in tissue and organs in the body other than the breast. Cell proliferative disorders of the breast may include hyperplasia, metaplasia, and dysplasia of the breast.

As used herein, “cell proliferative diseases or disorders of the skin” include all forms of cell proliferative disorders affecting skin cells. Cell proliferative disorders of the skin may include a precancer or precancerous condition of the skin, benign growths or lesions of the skin, melanoma, malignant melanoma or other malignant growths or lesions of the skin, and metastatic lesions in tissue and organs in the body other than the skin. Cell proliferative disorders of the skin may include hyperplasia, metaplasia, and dysplasia of the skin.

As used herein, “cell proliferative diseases or disorders of the endometrium” include all forms of cell proliferative disorders affecting cells of the endometrium. Cell proliferative disorders of the endometrium may include a precancer or precancerous condition of the endometrium, benign growths or lesions of the endometrium, endometrial cancer, and metastatic lesions in tissue and organs in the body other than the endometrium. Cell proliferative disorders of the endometrium may include hyperplasia, metaplasia, and dysplasia of the endometrium.

The bispecific compounds of formula (I) and their pharmaceutically acceptable salts and stereoisomers may be administered to a patient, e.g., a cancer patient, as a monotherapy or by way of combination therapy. Therapy may be “front/first-line”, i.e., as an initial treatment in patients who have undergone no prior anti-cancer treatment regimens, either alone or in combination with other treatments; or “second-line”, as a treatment in patients who have undergone a prior anti-cancer treatment regimen, either alone or in combination with other treatments; or as “third-line”, “fourth-line”, etc. treatments, either alone or in combination with other treatments. Therapy may also be given to patients who have had previous treatments which have been unsuccessful, or partially successful but who have become intolerant to the particular treatment. Therapy may also be given as an adjuvant treatment, i.e., to prevent reoccurrence of cancer in patients with no currently detectable disease or after surgical removal of a tumor. Therefore, in some embodiments, the compound may be administered to a patient who has received prior therapy, such as chemotherapy, radioimmunotherapy, surgical therapy, immunotherapy, radiation therapy, targeted therapy or any combination thereof.

The methods of the present invention may entail administration of a bispecific compound of formula (I) or a pharmaceutical composition thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5, or 6 weeks, and in other embodiments entails at least one 28-day cycle which includes daily administration for 3 weeks (21 days) followed by a 7-day “off” period. In other embodiments, the bispecific compound may be dosed twice a day (BID) over the course of two and a half days (for a total of 5 doses) or once a day (QD) over the course of two days (for a total of 2 doses). In other embodiments, the bispecific compound may be dosed once a day (QD) over the course of 5 days.

### Combination Therapy

The bispecific compounds of formula (I) and their pharmaceutically acceptable salts and stereoisomers may be used in combination or concurrently with at least one other active agent, e.g., anti-cancer agent or regimen, in treating diseases and disorders. The terms “in combination” and “concurrently” in this context mean that the agents are co-administered, which includes substantially contemporaneous administration, by way of the same or separate dosage forms, and by the same or different modes of administration, or sequentially, e.g., as part of the same treatment regimen, or by way of successive treatment regimens. Therefore, if given sequentially, at the onset of administration of the second compound, the first of the two compounds is in some cases still detectable at effective concentrations at the site of treatment. The sequence and time interval may be determined such that they can act together (e.g., synergistically) to provide an increased benefit than if they were administered otherwise. For example, the therapeutics may be administered at the same time or sequentially in any order at different points in time; however, if not administered at the same time, they may be administered sufficiently close in time so as to provide the desired therapeutic effect, which may be in a synergistic fashion. Therefore, the terms are not limited to the administration of the active agents at exactly the same time.

In some embodiments, the treatment regimen may include administration of a bispecific compound of formula (I) in combination with one or more additional therapeutics known for use in treating a disease or condition (e.g., cancer). The dosage of the additional therapeutic may be the same or even lower than known or recommended doses. See, Hardman et al., eds., Goodman & Gilman's The Pharmacological Basis Of Basis Of Therapeutics, 10th ed., McGraw-Hill, New York, 2001; Physician's Desk Reference 60th ed., 2006. For example, anti-cancer agents that may be suitable for use in combination with the inventive bispecific compounds are known in the art. See, e.g., U.S. Pat. No. 9,101,622 (Section 5.2 thereof) and U.S. Pat. No. 9,345,705 (Columns 12-18 thereof). Representative examples of additional anti-cancer agents and treatment regimens include radiation therapy, chemotherapeutics (e.g., mitotic inhibitors, angiogenesis inhibitors, anti-hormones, autophagy inhibitors, alkylating agents, intercalating antibiotics, growth factor inhibitors, anti-androgens, signal transduction pathway inhibitors, anti-microtubule agents, platinum coordination complexes, HDAC inhibitors, proteasome inhibitors, and topoisomerase inhibitors), immunomodulators, therapeutic antibodies (e.g., mono-specific and bispecific antibodies) and CAR-T therapy.

In some embodiments, a bispecific compound of formula (I) and the additional (e.g., anticancer) therapeutic may be administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. The two or more (e.g., anticancer) therapeutics may be administered within the same patient visit.

When the active components of the combination are not administered in the same pharmaceutical composition, it is understood that they can be administered in any order to a subject in need thereof. For example, a bispecific compound of the present invention can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the additional therapeutic, to a subject in need thereof. In various aspects, the therapeutics are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one example, the (e.g., anticancer) therapeutics are administered within the same office visit. In another example, the combination anticancer therapeutics may be administered at 1 minute to 24 hours apart.

In some embodiments involving cancer treatment, a bispecific compound of formula (I) and the additional anti-cancer agent or therapeutic are cyclically administered. Cycling therapy involves the administration of one anticancer therapeutic for a period of time, followed by the administration of a second anti-cancer therapeutic for a period of time and repeating this sequential administration, i.e., the cycle, in order to reduce the development of resistance to one or both of the anticancer therapeutics, to avoid or reduce the side effects of one or both of the anticancer therapeutics, and/or to improve the efficacy of the therapies. In one example, cycling therapy involves the administration of a first anticancer therapeutic for a period of time, followed by the administration of a second anticancer therapeutic for a period of time, optionally, followed by the administration of a third anticancer therapeutic for a period of time and so forth, and repeating this sequential administration, i.e., the cycle in order to reduce the development of resistance to one of the anticancer therapeutics, to avoid or reduce the side effects of one of the anticancer therapeutics, and/or to improve the efficacy of the anticancer therapeutics.

In some embodiments, the bispecific compound of the present invention may be used in combination with other anti-cancer agents, examples of which include Paclitaxel (e.g., ovarian cancer, breast cancer, lung cancer, Kaposi sarcoma, cervical cancer, and pancreatic cancer), Topotecan (e.g., ovarian cancer and lung cancer), Irinotecan (e.g., colon cancer, and small cell lung cancer), Etoposide (e.g., testicular cancer, lung cancer, lymphomas, and non-lymphocytic leukemia), Vincristine (e.g., leukemia), Leucovorin (e.g., colon cancer), Altretamine (e.g., ovarian cancer), Daunorubicin (e.g., acute myeloid leukemia (AML), acute lymphocytic leukemia (ALL), chronic myelogenous leukemia (CML), and Kaposi's sarcoma), Trastuzumab (e.g., breast cancer, stomach cancer, and esophageal cancer), Rituximab (e.g., non-Hodgkin's lymphoma), Cetuximab (e.g., colorectal cancer, metastatic non-small cell lung cancer and head and neck cancer), Pertuzumab (e.g., metastatic HER2-positive breast cancer), Alemtuzumab (e.g., chronic lymphocytic leukemia (CLL), cutaneous T-cell lymphoma (CTCL) and T-cell lymphoma), Panitumumab (e.g., colon and rectum cancer), Tamoxifen (e.g., breast cancer), Fulvestrant (e.g., breast cancer), Letrazole (e.g., breast cancer), Exemestane (e.g., breast cancer), Azacytidine (e.g., myelodysplastic syndromes), Mitomycin C (e.g., gastro-intestinal cancers, anal cancers, and breast cancers), Dactinomycin (e.g., Wilms tumor, rhabdomyosarcoma, Ewing's sarcoma, trophoblastic neoplasm, testicular cancer, and ovarian cancer), Erlotinib (e.g., non-small cell lung cancer and pancreatic cancer), Sorafenib (e.g., kidney cancer and liver cancer), Temsirolimus (e.g., kidney cancer), Bortezomib (e.g., multiple myeloma and mantle cell lymphoma), Pegaspargase (e.g., acute lymphoblastic leukemia), Cabometyx (e.g., hepatocellular carcinoma, medullary thyroid cancer, and renal cell carcinoma), Keytruda (e.g., cervical cancer, gastric cancer, hepatocellular carcinoma, Hodgkin lymphoma, melanoma, Merkel cell carcinoma, non-small cell lung cancer, urothelial carcinoma, and squamous cell carcinoma of the head and neck), Nivolumab (e.g., colorectal cancer, hepatocellular carcinoma, melanoma, non-small cell lung cancer, renal cell carcinoma, small cell lung cancer, and urothelial carcinoma), Regorafenib (e.g., colorectal cancer, gastrointestinal stromal tumor, and hepatocellular carcinoma), and dexamethasone (e.g., acute multiple myeloma).

### Pharmaceutical Kits

The present bispecific compounds and/or compositions containing them may be assembled into kits or pharmaceutical systems. Kits or pharmaceutical systems according to this aspect of the invention include a carrier or package such as a box, carton, tube or the like, having in close confinement therein one or more containers, such as vials, tubes, ampoules, or bottles, which contain a bispecific compound of formula (I) or a pharmaceutical composition thereof. The kits or pharmaceutical systems of the invention may also include printed instructions for using the bispecific compounds and compositions.

These and other aspects of the present invention will be further appreciated upon consideration of the following Examples, which are intended to illustrate certain particular embodiments of the invention but are not intended to limit its scope, as defined by the claims.

### EXAMPLES

### Example 1: Synthesis of Ceritinib Analog Core

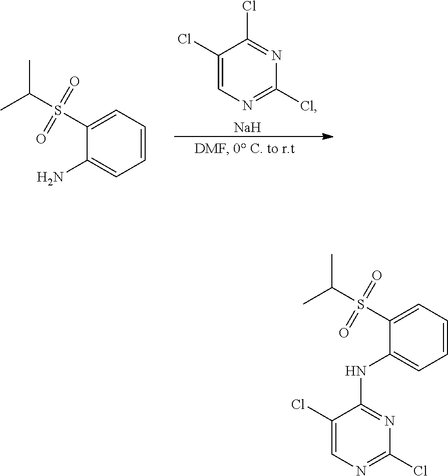

### 2,5-dichloro-N-(2-(isopropylsulfonyl)phenyl)pyrimidin-4-amine

NaH (60%, 30.1 g, 0.753 mmol) was added to a solution of 2-(isopropylsulfonyl)aniline (75.0 g, 0.376 mol) in 300 mL of DMF at 0° C. After stirring at 0° C. for 0.5 h, 2,4,5-trichloropyrimidine (82.8 g, 0.452 mol) was added and the reaction mixture stirred at room temperature overnight. The reaction was monitored by thin-layer chromatography (TLC). The reaction mixture was diluted in water at 0° C. and extracted with EtOAc×2. The combined organic phases were dried with Na₂SO₄ and concentrated in vacuo, and the residue was purified by silica gel column (Pet. Ether/DCM/EtOAc=20/4/1, v/v/v) to give the title compound as a white solid (62 g, 48%). ¹H NMR (400 MHz, CDCl₃): δ 9.81 (s, 1H), 8.55 (s, 1H), 8.31 (d, J=8.4 Hz, 1H), 7.91-7.82 (m, 2H), 7.48 (t, J=7.6 Hz, 1H), 1.16 (d, J=6.8 Hz, 6H). ESI-MS (EI⁺, m/z): 346.1.

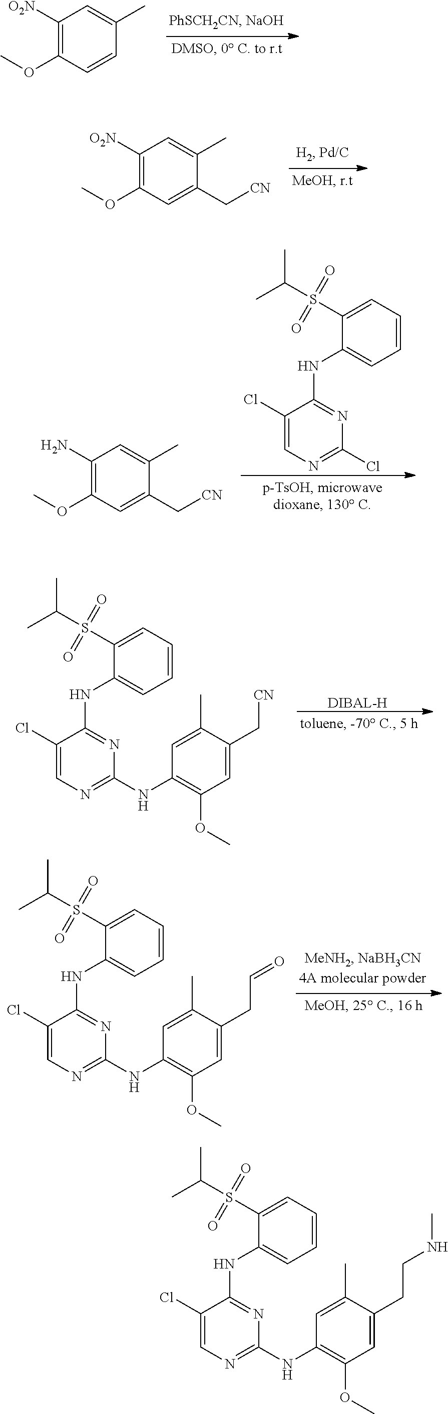

### 2-(5-methoxy-2-methyl-4-nitrophenyl)acetonitrile

A mixture of NaOH (60 g, 1.20 mol) in DMSO (50 mL) was added to a solution of 1-methoxy-4-methyl-2-nitrobenzene (25.0 g, 0.120 mol) and PhSCH₂CN (22.3 g, 0.120 mol) in DMSO (50 mL) at 0° C. After stirring at room temperature overnight, the mixture was diluted in 6 M HCl at 0° C. and the mixture was extracted with EtOAc×3. The combined organic phases were dried with Na₂SO₄ and concentrated in-vacuo, and the residue was purified by silica gel column (Pet. Ether/EtOAc=10/1, v/v) to afford the title compound as a yellow solid (24.0 g, 78%). ¹H NMR (400 MHz, CDCl₃): δ 8.63 (d, J=7.2 Hz, 1H), 8.55 (s, 1H), 7.00 (d, J=7.2 Hz, 1H), 4.42 (q, J=7.2 Hz, 2H), 1.41 (t, J=6.8 Hz, 3H). ESI-MS (EI⁺, m/z): 207.1.

### 2-(4-amino-5-methoxy-2-methylphenyl)acetonitrile

Pd/C (10%, 4 g) was added to a solution of 2-(5-methoxy-2-methyl-4-nitrophenyl)acetonitrile (24.0 g, 0.113 mol) in MeOH (600 mL). After stirring at room temperature under H₂ for 72 hours, TLC showed that most of the starting material was consumed. The reaction mixture was filtered and the filtrate was concentrated in-vacuo to give the title compound as a red solid (20.2 g, 96%). ¹H NMR (400 MHz, CDCl₃): δ 6.75 (s, 1H), 6.48 (s, 1H), 4.69 (brs, 1H), 3.74 (s, 2H), 3.73 (s, 3H), 2.11 (s, 3H). ESI-MS (EI⁺, m/z): 177.1.

### 2-(4-((5-chloro-4-((2-(isopropylsulfonyl)phenyl)amino)pyrimidin-2-yl)amino)-5-methoxy-2-methylphenyl)acetonitrile

p-TsOH (176 mg, 0.92 mmol) was added to a solution of 2,5-dichloro-N-(2-(isopropylsulfonyl)phenyl)pyrimidin-4-amine (1.6 g, 4.62 mmol) and 2-(4-amino-5-methoxy-2-methylphenyl)acetonitrile (896 mg, 2.54 mmol) in 1,4-dioxane (16 mL). After reacting under microwave conditions at 130° C. for 4 hours, the reaction mixture was filtered and the solids were washed with MeOH. The filtrate was diluted with sat. NaHCO₃ aq. and stirred at room temperature overnight. The mixture was filtered to afford the title compound as a brown solid (1.57 g, 70%). ¹H NMR (400 MHz, DMSO-d₆): δ 9.49 (s, 1H), 8.45 (d, J=4.4 Hz, 1H), 8.34 (s, 1H), 8.25 (s, 1H), 7.83 (dd, J=1.6, 8.0 Hz, 1H), 7.63 (t, J=8.8 Hz, 1H), 7.58 (s, 1H), 7.34 (t, J=4.4 Hz, 1H), 7.04 (s, 1H), 3.93 (s, 2H), 3.78 (s, 3H), 3.40-3.47 (m, 1H), 2.16 (s, 3H), 1.15 (d, J=6.8 Hz, 6H). ESI-MS (EI⁺, m/z): 486.1.

### 2-(4-((5-chloro-4-((2-(isopropylsulfonyl)phenyl)amino)pyrimidin-2-yl)amino)-5-methoxy-2-methylphenyl)acetaldehyde

DIBAL-H (1 M, 50 mL, 50.0 mmol) was added to a solution of 2-(4-((5-chloro-4-((2-(isopropylsulfonyl)phenyl)amino)pyrimidin-2-yl)amino)-5-methoxy-2-methylphenyl)acetonitrile (4.85 g, 10.0 mmol) in toluene (100 mL) was added at −65° C. under nitrogen. After stirring at room temperature overnight, TLC showed that the starting material was consumed. The reaction mixture was quenched by addition of water at 0° C., and the mixture was extracted with EtOAc×3. The combined organic phases were dried and concentrated in-vacuo to a light yellow solid (4.88 g, 100%). ESI-MS (EI⁺, m/z): 489.1. The crude product was used without further purification in the next step.

### 5-chloro-N4-(2-(isopropylsulfonyl)phenyl)-N2-(2-methoxy-5-methyl-4-(2-(methylamino)ethyl)phenyl)pyrimidine-2,4-diamine

A solution of 2-(4-((5-chloro-4-((2-(isopropylsulfonyl)phenyl)amino)pyrimidin-2-yl)amino)-5-methoxy-2-methylphenyl)acetaldehyde (4.88 g, 10.0 mmol), MeNH₂ (2 M, 25 mL, 50 mmol) in MeOH (50 mL) stirred at rt. NaBH₃CN (1.86 g, 30.0 mmol) was added and the reaction mixture stirred in an ice bath for 16 hours. LCMS showed that the reaction reached completion. The mixture was partitioned between ethyl acetate and water and extracted with EtOAc×2. The combined organic phases were washed with water and brine, dried over Na₂SO₄ and concentrated in-vacuo. The crude product was purified by column chromatography eluted with DCM/MeOH=10/1 to afford the title compound as a light yellow solid (1.05 g, 20.8%). ESI-MS (EI⁺, m/2z): 504.2.

### Example 2: Synthesis of N-(4-((5-chloro-4-((2-(isopropyl-(methylene)sulfinyl)phenyl)amino)pyrimidin-2-yl)amino)-5-methoxy-2-methylphenethyl)-4-((2-(2,6-dioxopiperidin-3-yl)-1,3-dioxoisoindolin-4-yl)amino)-N-methylbutanamide (1)

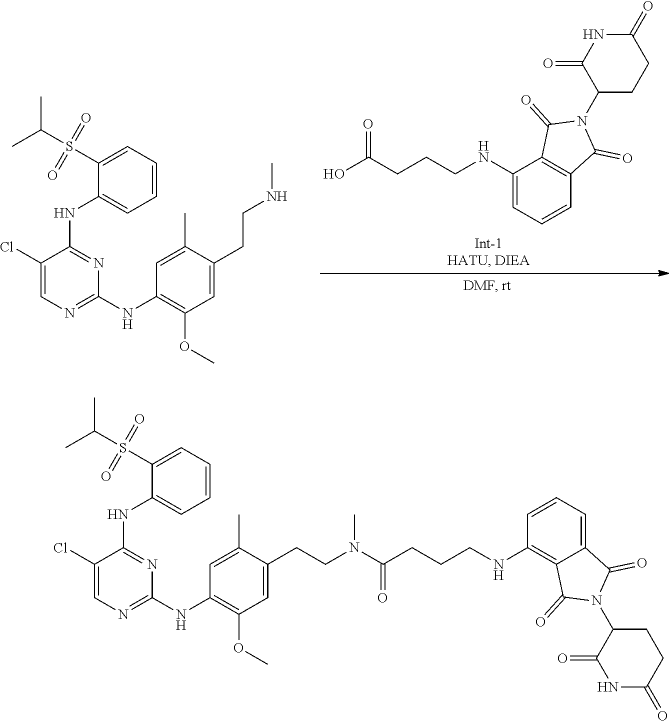 [SMILES: CNCCc1cc(OC)c(Nc2ncc(Cl)c(Nc3ccccc3S(=O)(=O)C(C)C)n2)cc1C ; O=C(O)CCCNc1cccc2c1C(=O)N(C1CCC(=O)NC1=O)C2=O ; COc1cc(CCN(C)C(=O)CCCNc2cccc3c2C(=O)N(C2CCC(=O)NC2=O)C3=O)c(C)cc1Nc1ncc(Cl)c(Nc2ccccc2S(=O)(=O)C(C)C)n1]

5-chloro-N4-(2-(isopropylsulfonyl)phenyl)-N2-(2-methoxy-5-methyl-4-(2-(methylamino)ethyl)phenyl)pyrimidine-2,4-diamine (80 mg, 0.159 mmol) was added to a solution of Int-1 (64 mg, 0.177 mmol), DIEA (62 mg, 0.483 mmol) and HATU (92 mg, 0.241 mmol) in DMF (5 mL). After stirring at rt for 2 h, the reaction mixture was diluted with EtOAc and washed with water and brine. The organic phase was dried with Na₂SO₄ and concentrated in-vacuo. The concentrate was purified by prep-HPLC to afford the title compound as a yellow solid (24.5 mg, 18.3%). ¹H NM/R (400 MHz, DMSO-d₆): δ 11.10 (s, 1H), 9.56 (s, 1H), 8.55-8.25 (m, 2H), 8.25-8.22 (in, 1H), 7.84-7.81 (in, 1H), 7.67-7.44 (m, 3H), 7.38-7.33 (in, 1H), 7.21-6.98 (m, 2H), 6.96-6.80 (in, 1H), 6.73-6.48 (in, 1H), 5.05-5.02 (in, 1H), 3.77-3.72 (m, 4H), 3.47-3.35 (m, 3H), 3.35-3.25 (m, 1H), 3.03-2.95 (m, 1H), 2.95-2.89 (m, 4H), 2.73-2.66 (m, 2H), 2.58-2.51 (m, 2H), 2.37-2.33 (m, 1H), 2.16-1.82 (m, 5H), 1.82-1.79 (m, 1H), 1.54-1.49 (m, 1H), 1.19-1.16 (m, 6H). ESI-MS (EI⁺, m/2z): 845.2.

### Example 3: Synthesis of N-(4-((5-chloro-4-((2-(isopropylsulfonyl)phenyl)amino)pyrimidin-2-yl)amino)-5-methoxy-2-methylphenethyl)-6-((2-(2,6-dioxopiperidin-3-yl)-1,3-dioxoisoindolin-4-yl)amino)-N-methylhexanamide (2)

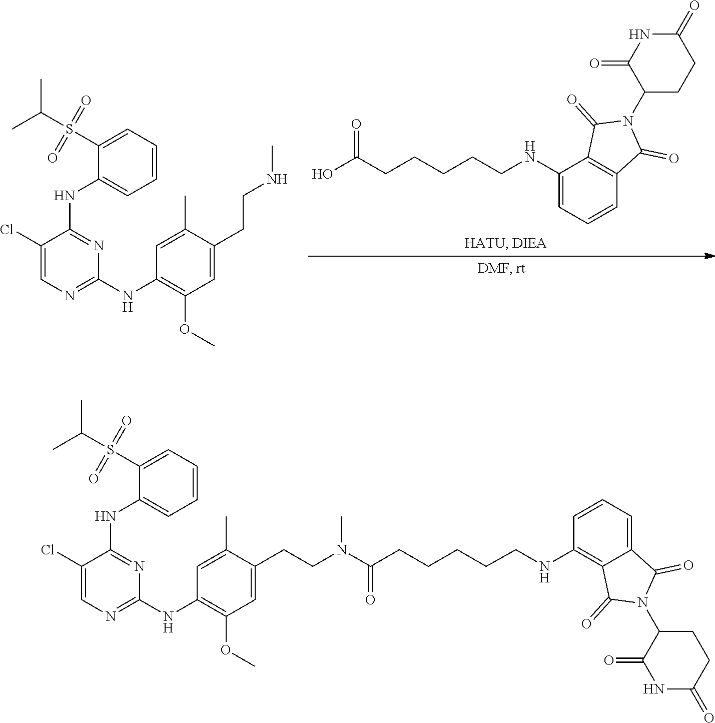 [SMILES: CNCCc1cc(OC)c(Nc2ncc(Cl)c(Nc3ccccc3S(=O)(=O)C(C)C)n2)cc1C ; O=C(O)CCCCCNc1cccc2c1C(=O)N(C1CCC(=O)NC1=O)C2=O ; COc1cc(CCN(C)C(=O)CCCCCNc2cccc3c2C(=O)N(C2CCC(=O)NC2=O)C3=O)c(C)cc1Nc1ncc(Cl)c(Nc2ccccc2S(=O)(=O)C(C)C)n1]

Compound 2 was synthesized based on similar procedure as compound 1. ¹H NMR (400 MHz, DMSO-d₆): δ 11.09 (s, 1H), 9.58 (d, J=8.0 Hz, 1H), 7.85-7.82 (m, 1H), 7.67-7.35 (m, 4H), 7.07-6.98 (m, 2H), 6.82-6.80 (m, 1H), 6.54-6.45 (m, 1H), 5.05-5.02 (m, 1H), 4.51-4.48 (m, 1H), 3.76-3.73 (m, 3H), 3.45-3.19 (m, 5H), 2.95-2.69 (m, 6H), 2.59-2.55 (m, 2H), 2.32-2.27 (m, 1H), 2.27-2.13 (m, 2H), 2.04-2.01 (m, 2H), 1.61-1.36 (m, 5H), 1.15-1.13 (m, 6H). ESI-MS (EI⁺, m/2z): 437.2.

### Example 4: Synthesis of N-(4-((5-chloro-4-((2-(isopropylsulfonyl)phenyl)amino)pyrimidin-2-yl)amino)-5-methoxy-2-methylphenethyl)-9-((2-(2,6-dioxopiperidin-3-yl)-1,3-dioxoisoindolin-4-yl)amino)-N-methylnonanamide (3)

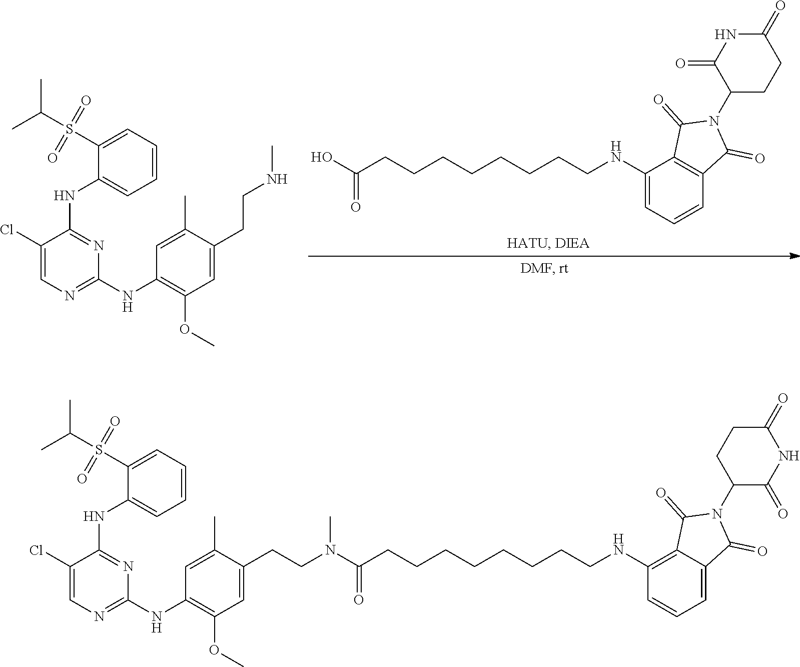 [SMILES: CNCCc1cc(OC)c(Nc2ncc(Cl)c(Nc3ccccc3S(=O)(=O)C(C)C)n2)cc1C ; O=C(O)CCCCCCCCNc1cccc2c1C(=O)N(C1CCC(=O)NC1=O)C2=O ; COc1cc(CCN(C)C(=O)CCCCCCCCNc2cccc3c2C(=O)N(C2CCC(=O)NC2=O)C3=O)c(C)cc1Nc1ncc(Cl)c(Nc2ccccc2S(=O)(=O)C(C)C)n1]

Compound 3 was synthesized based on similar procedure as compound 1. ¹H NMR (400 MHz, DMSO-d₆): δ 11.10 (s, 1H), 9.52 (s, 1H), 8.54-8.44 (m, 1H), 8.39-8.22 (m, 2H), 7.95-7.05 (m, 1H), 7.68-7.44 (m, 3H), 7.35-7.30 (m, 1H), 7.10-6.98 (m, 2H), 6.82 (d, J=13.6 Hz, 1H), 6.56-6.45 (m, 1H), 5.06-5.02 (m, 1H), 3.76-3.71 (m, 3H), 3.40-3.37 (m, 1H), 3.32-3.18 (m, 2H), 2.96-2.83 (m, 4H), 2.81-2.74 (m, 1H), 2.74-2.66 (m, 1H), 2.67-2.53 (m, 2H), 2.46-2.29 (m, 1H), 2.28-2.22 (m, 1H), 2.17-1.93 (m, 3H), 1.60-1.40 (m, 3H), 1.38-1.10 (m, 15H). ESI-MS (EI⁺, m/2z): 458.8.

### Example 5: Synthesis of N-(4-((5-chloro-4-((2-(isopropylsulfonyl)phenyl)amino)pyrimidin-2-yl)amino)-5-methoxy-2-methylphenethyl)-3-(2-(2-((2-(2,6-dioxopiperidin-3-yl)-1,3-dioxoisoindolin-4-yl)amino)ethoxy)ethoxy)-N-methylpropanamide (4)

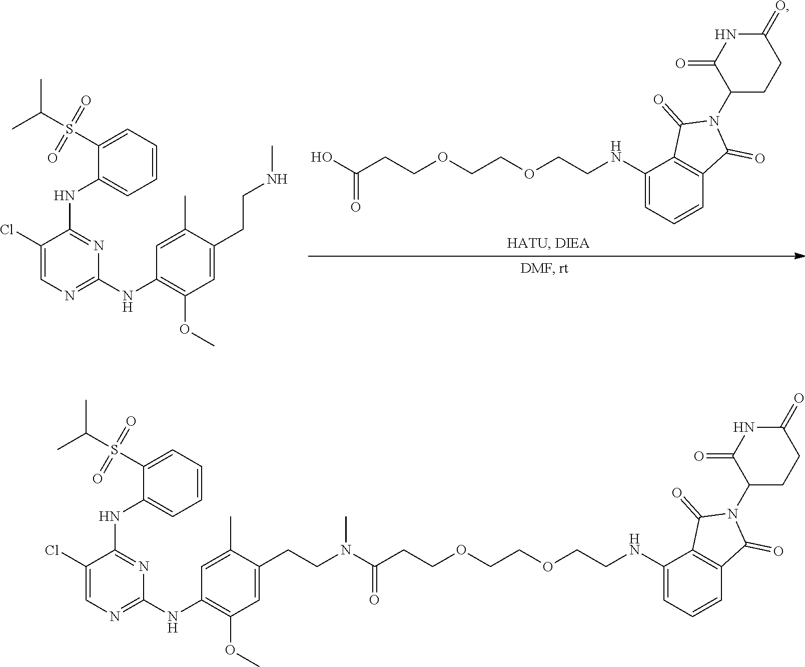 [SMILES: CNCCc1cc(OC)c(Nc2ncc(Cl)c(Nc3ccccc3S(=O)(=O)C(C)C)n2)cc1C ; O=C(O)CCOCCOCCNc1cccc2c1C(=O)N(C1CCC(=O)NC1=O)C2=O ; COc1cc(CCN(C)C(=O)CCOCCOCCNc2cccc3c2C(=O)N(C2CCC(=O)NC2=O)C3=O)c(C)cc1Nc1ncc(Cl)c(Nc2ccccc2S(=O)(=O)C(C)C)n1]

Compound 4 was synthesized based on similar procedure as compound 1. ¹H NMR (400 MHz, DMSO-d₆): δ 11.09 (s, 1H), 9.48 (s, 1H), 8.51 (d, J=3.6 Hz, 1H), 8.25-8.20 (m, 2H), 7.81 (d, J=8.0 Hz, 1H), 7.66-7.45 (m, 3H), 7.34 (t, J=11.6 Hz, 1H), 7.11 (dd, J=8.4, 17.2 Hz, 1H), 7.02 (t, J=7.6 Hz, 1H), 6.85-6.82 (m, 1H), 6.63-6.54 (m, 3H), 5.04 (quint, J=6.0 Hz, 1H), 3.78-3.72 (m, 3H), 3.66-3.37 (m, 13H), 2.96-2.74 (m, 5H), 2.72-2.65 (m, 1H), 2.62-2.56 (m, 1H), 2.54-2.51 (m, 1H), 2.39 (t, J=6.4 Hz, 1H), 2.15 (d, J=8.0 Hz, 3H), 2.10-1.94 (m, 2H), 1.15 (d, J=6.4 Hz, 6H). ESI-MS (EI⁺, m/2z): 460.2.

### Example 6: Synthesis of N-(4-((5-chloro-4-((2-(isopropylsulfonyl)phenyl)amino)pyrimidin-2-yl)amino)-5-methoxy-2-methylphenethyl)-4-(2-((2-(2,6-dioxopiperidin-3-yl)-1,3-dioxoisoindolin-4-yl)oxy)acetamido)-N-methylbutanamide (5)

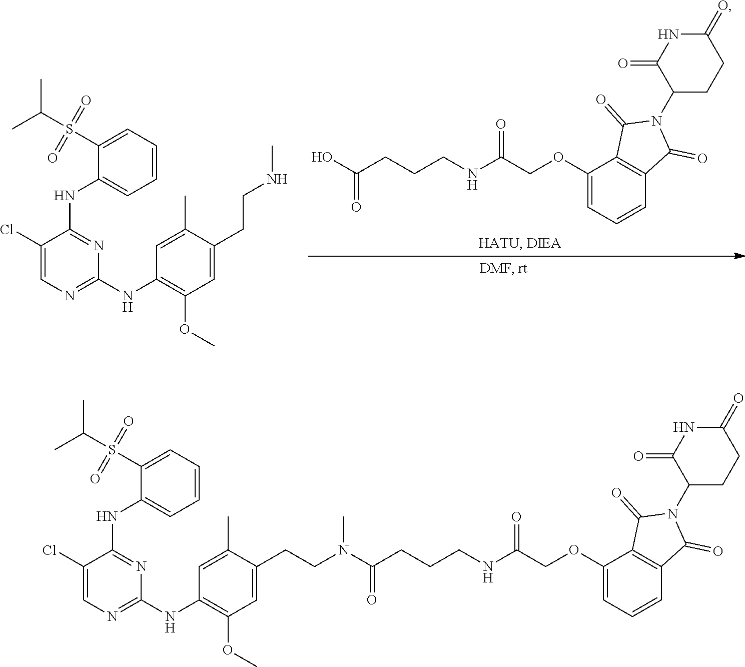 [SMILES: CNCCc1cc(OC)c(Nc2ncc(Cl)c(Nc3ccccc3S(=O)(=O)C(C)C)n2)cc1C ; O=C(O)CCCNC(=O)COc1cccc2c1C(=O)N(C1CCC(=O)NC1=O)C2=O ; COc1cc(CCN(C)C(=O)CCCNC(=O)COc2cccc3c2C(=O)N(C2CCC(=O)NC2=O)C3=O)c(C)cc1Nc1ncc(Cl)c(Nc2ccccc2S(=O)(=O)C(C)C)n1]

Compound 5 was synthesized based on similar procedure as compound 1. ¹H NMR (400 MHz, DMSO-d₆): δ 11.12 (d, J=4.0 Hz, 1H), 9.60 (d, J=8.8 Hz, 1H), 8.48-8.37 (m, 2H), 8.25 (d, J=8.4 Hz, 1H), 8.00-7.89 (m, 1H), 7.86-7.73 (m, 2H), 7.68-7.62 (m, 1H), 7.51-7.33 (m, 4H), 6.94 (d, J=5.2 Hz, 1H), 5.15-5.06 (m, 1H), 4.79-4.73 (m, 2H), 3.78-3.71 (m, 3H), 3.49-3.37 (m, 3H), 3.22-3.14 (m, 1H), 3.13-3.05 (m, 1H), 2.94-2.81 (m, 4H), 2.80-2.74 (m, 1H), 2.73-2.65 (m, 1H), 2.63-2.53 (m, 1H), 2.34-2.27 (m, 1H), 2.20-2.10 (m, 4H), 2.08-1.95 (m, 2H), 1.72-1.64 (m, 1H), 1.62-1.53 (m, 1H), 1.15 (d, J=7.6 Hz, 6H). ESI-MS (EI⁺, m/z): 903.2.

### Example 7: Synthesis of N-(4-((5-chloro-4-((2-(isopropylsulfonyl)phenyl)amino)pyrimidin-2-yl)amino)-5-methoxy-2-methylphenethyl)-6-(2-((2-(2,6-dioxopiperidin-3-yl)-1,3-dioxoisoindolin-4-yl)oxy)acetamido)-N-methylhexanamide (6)

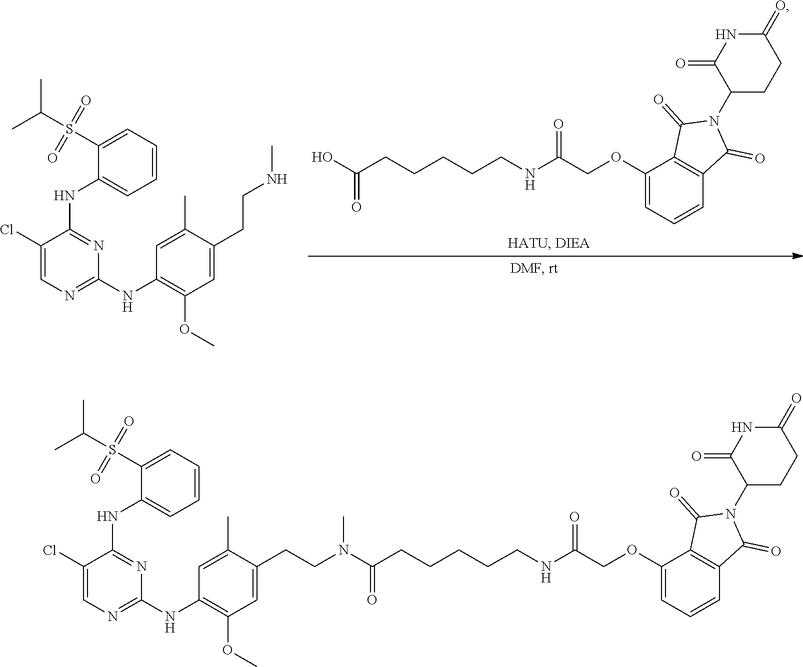 [SMILES: CNCCc1cc(OC)c(Nc2ncc(Cl)c(Nc3ccccc3S(=O)(=O)C(C)C)n2)cc1C ; O=C(O)CCCCCNC(=O)COc1cccc2c1C(=O)N(C1CCC(=O)NC1=O)C2=O ; COc1cc(CCN(C)C(=O)CCCCCNC(=O)COc2cccc3c2C(=O)N(C2CCC(=O)NC2=O)C3=O)c(C)cc1Nc1ncc(Cl)c(Nc2ccccc2S(=O)(=O)C(C)C)n1]

Compound 6 was synthesized based on similar procedure as compound 1. ¹H NMR (400 MHz, DMSO-d₆): δ 11.12 (brs, 1H), 9.55 (d, J=4.0 Hz, 1H), 8.50-8.29 (m, 2H), 8.26-8.23 (m, 1H), 7.96-7.86 (m, 1H), 7.85-7.76 (m, 2H), 7.68-7.61 (m, 1H), 7.51-7.32 (m, 4H), 6.83 (d, J=2.8 Hz, 1H), 5.15-5.07 (m, 1H), 4.79-4.72 (m, 2H), 3.78-3.74 (m, 3H), 3.43-3.38 (m, 3H), 3.18-3.03 (m, 2H), 2.96-2.82 (m, 4H), 2.80-2.75 (m, 1H), 2.73-2.66 (m, 1H), 2.63-2.53 (m, 2H), 2.26 (t, J=7.2 Hz, 1H), 2.17-1.97 (m, 5H), 1.55-1.21 (m, 6H), 1.15 (d, J=6.8 Hz, 6H). ESI-MS (EI⁺, m/z): 931.2.

### Example 8: Synthesis of N-(4-((5-chloro-4-((2-(isopropylsulfonyl)phenyl)amino)pyrimidin-2-yl)amino)-5-methoxy-2-methylphenethyl)-9-(2-((2-(2,6-dioxopiperidin-3-yl)-1,3-dioxoisoindolin-4-yl)oxy)acetamido)-N-methylnonanamide (7)

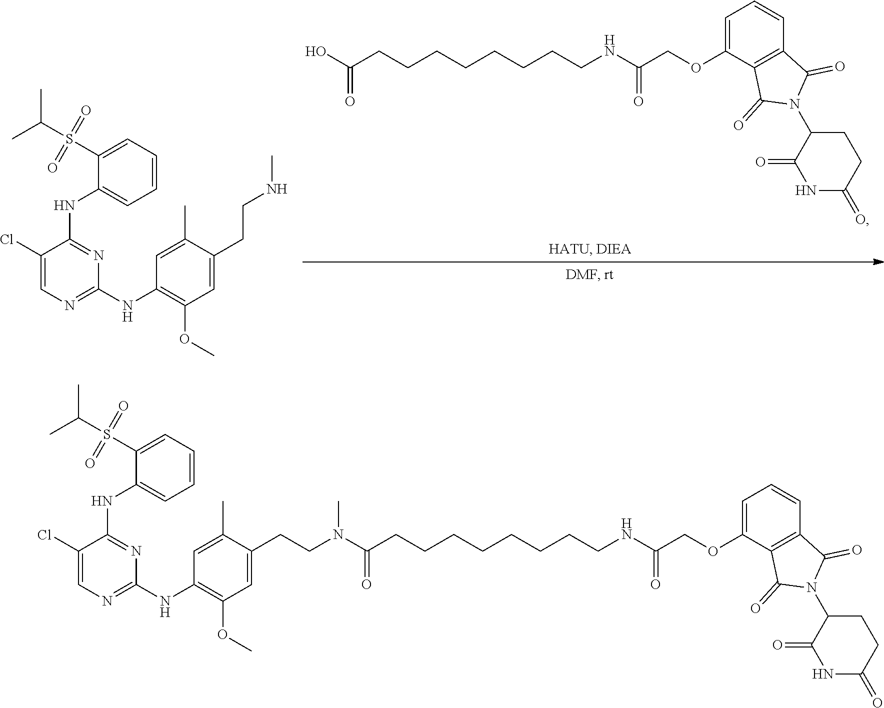 [SMILES: CNCCc1cc(OC)c(Nc2ncc(Cl)c(Nc3ccccc3S(=O)(=O)C(C)C)n2)cc1C ; O=C(O)CCCCCCCCNC(=O)COc1cccc2c1C(=O)N(C1CCC(=O)NC1=O)C2=O ; COc1cc(CCN(C)C(=O)CCCCCCCCNC(=O)COc2cccc3c2C(=O)N(C2CCC(=O)NC2=O)C3=O)c(C)cc1Nc1ncc(Cl)c(Nc2ccccc2S(=O)(=O)C(C)C)n1]

Compound 7 was synthesized based on similar procedure as compound 1. ¹H NMR (400 MHz, DMSO-d₆): δ 11.12 (s, 1H), 9.56 (s, 1H), 9.51-9.29 (i, 2H), 8.26 (s, 1H), 7.95-7.87 (m, 1H), 7.86-7.76 (m, 2H), 7.69-7.59 (m, 1H), 7.53-7.43 (m, 2H), 7.41-7.32 (m, 2H), 6.85-6.80 (M, 0.7H), 6.71 (s, 0.2H), 6.55 (s, 0.1H), 5.15-5.08 (i, 1H), 4.78-4.73 (m, 2H), 4.52-4.46 (m, 1H), 3.78-3.71 (m, 3H), 3.49-3.38 (m, 2H), 3.17-3.06 (m, 2H), 2.96-2.82 (m, 4H), 2.81-2.66 (m, 1.5H), 2.63-2.53 (m, 1.5H), 2.43-2.24 (m, 2H), 2.16-1.96 (m, 5H), 1.56-1.05 (m, 18H). ESI-MS (EI⁺, m/2z): 487.3.

### Example 9: Synthesis of N-(4-((5-chloro-4-((2-(isopropylsulfonyl)phenyl)amino)pyrimidin-2-yl)amino)-5-methoxy-2-methylphenethyl)-3-(2-(2-(2-((2-(2,6-dioxopiperidin-3-yl)-1,3-dioxoisoindolin-4-yl)oxy)acetamido)ethoxy)ethoxy)-N-methylpropanamide (8)

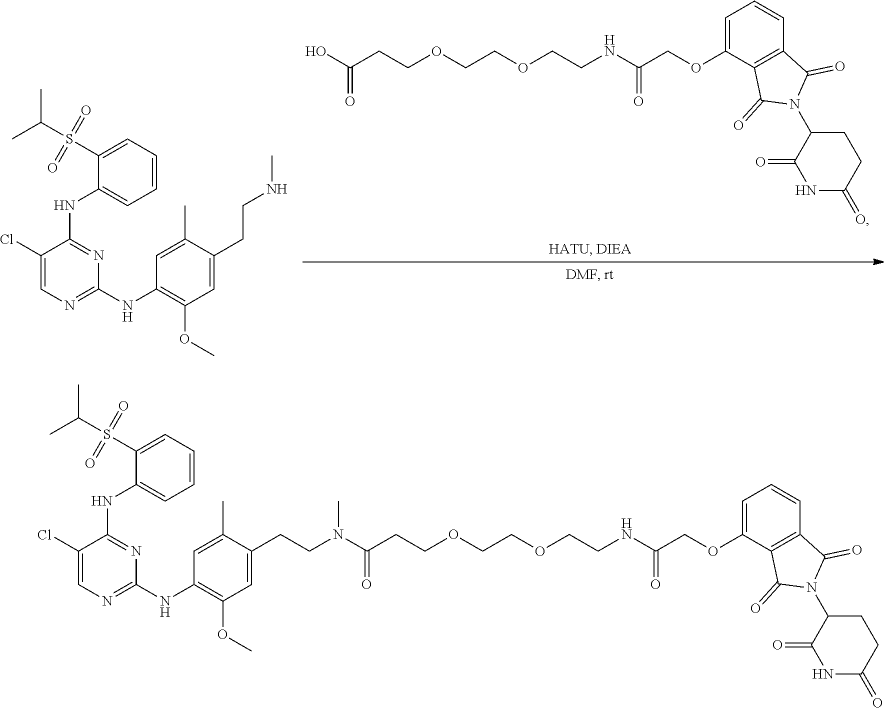 [SMILES: CNCCc1cc(OC)c(Nc2ncc(Cl)c(Nc3ccccc3S(=O)(=O)C(C)C)n2)cc1C ; O=C(O)CCOCCOCCNC(=O)COc1cccc2c1C(=O)N(C1CCC(=O)NC1=O)C2=O ; COc1cc(CCN(C)C(=O)CCOCCOCCNC(=O)COc2cccc3c2C(=O)N(C2CCC(=O)NC2=O)C3=O)c(C)cc1Nc1ncc(Cl)c(Nc2ccccc2S(=O)(=O)C(C)C)n1]

Compound 8 was synthesized based on similar procedure as compound 1. ¹H NMR (400 MHz, DMSO-d₆): δ 11.11 (s, 1H), 9.48 (s, 1H), 8.50 (d, J=5.2 Hz, 1H), 8.27-8.21 (m, 2H), 8.03-7.95 (m, 3H), 7.84-7.76 (m, 1H), 7.67-7.59 (m, 1H), 7.52-7.45 (m, 2H), 7.41-7.30 (m, 2H), 6.96-6.92 (m, 1H), 5.15-5.07 (m, 1H), 4.80-4.75 (m, 2H), 3.79-3.73 (m, 3H), 3.63 (t, J=6.8 Hz, 1H), 3.57-3.37 (m, 10H), 3.32-3.25 (m, 2H), 2.98-2.75 (m, 5H), 2.72-2.66 (m, 1H), 2.64-2.52 (m, 3H), 2.40 (t, J=6.4 Hz, 1H), 2.15 (d, J=8.0 Hz, 3H), 2.08-1.97 (m, 2H), 1.15 (d, J=6.4 Hz, 6H). ESI-MS (EI⁺, m/z): 978.2.

### Example 10: Synthesis of N-(4-((5-chloro-4-((2-(isopropylsulfonyl)phenyl)amino)pyrimidin-2-yl)amino)-5-methoxy-2-methylphenethyl)-6-(2-(2,6-dioxopiperidin-3-yl)-1,3-dioxoisoindolin-4-yl)-N-methylhex-5-ynamide (9)

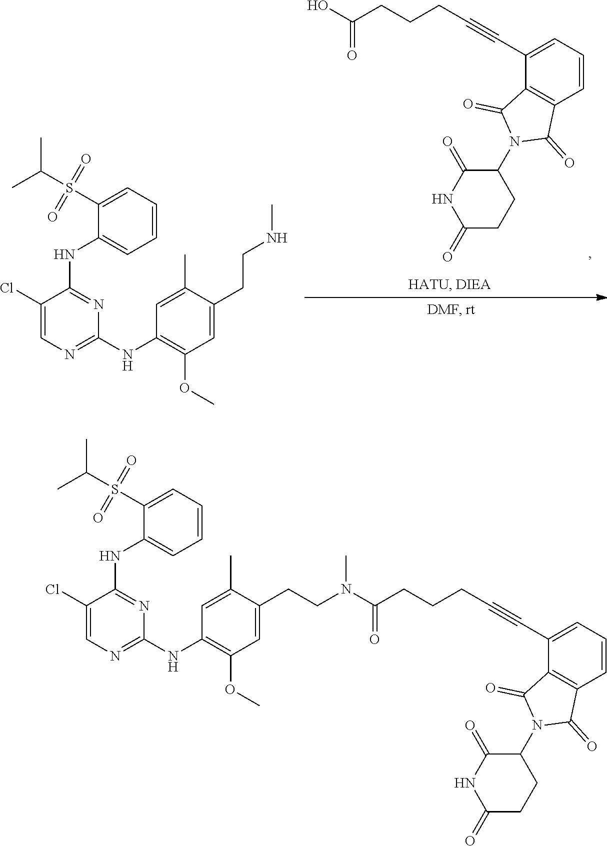 [SMILES: CNCCc1cc(OC)c(Nc2ncc(Cl)c(Nc3ccccc3S(=O)(=O)C(C)C)n2)cc1C ; O=C(O)CCCC#Cc1cccc2c1C(=O)N(C1CCC(=O)NC1=O)C2=O ; COc1cc(CCN(C)C(=O)CCCC#Cc2cccc3c2C(=O)N(C2CCC(=O)NC2=O)C3=O)c(C)cc1Nc1ncc(Cl)c(Nc2ccccc2S(=O)(=O)C(C)C)n1]

Compound 9 was synthesized based on similar procedure as compound 1. ¹H NMR (400 MHz, DMSO-d₆): δ 11.13 (s, 1H), 9.46 (s, 1H), 8.55-8.45 (m, 1H), 8.25-8.19 (m, 2H), 7.90-7.72 (m, 4H), 7.67-7.60 (m, 1H), 7.48 (s, 1H), 7.37-7.30 (m, 1H), 6.86-6.79 (m, 1H), 5.19-5.10 (m, 1H), 3.79-3.72 (m, 3H), 3.53-3.39 (m, 3H), 2.99 (s, 1H), 2.96-2.83 (m, 3H), 2.83-2.77 (m, 1H), 2.76-2.69 (m, 1H), 2.65-2.53 (m, 2H), 2.47-2.42 (m, 1H), 2.37-2.31 (m, 1H), 2.25-2.12 (m, 4H), 2.11-1.95 (m, 2H), 1.85-1.76 (m, 1H), 1.66-1.57 (m, 1H), 1.17-1.11 (m, 6H). ESI-MS (EI⁺, m/z): 854.2.

### Example 11: Synthesis of N-(4-((5-chloro-4-((2-(isopropylsulfonyl)phenyl)amino)pyrimidin-2-yl)amino)-5-methoxy-2-methylphenethyl)-8-(2-(2,6-dioxopiperidin-3-yl)-1,3-dioxoisoindolin-4-yl)-N-methyloct-7-ynamide (10)

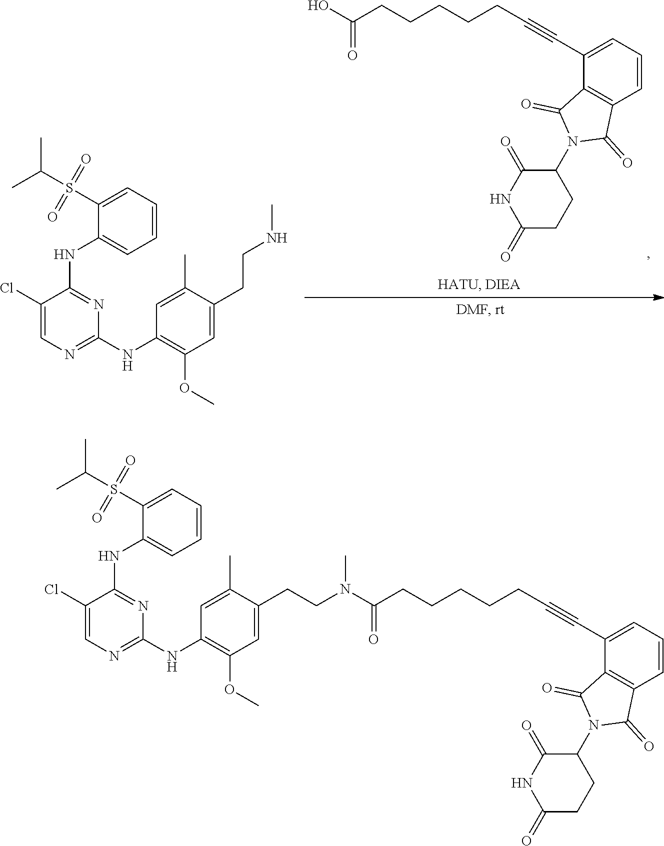 [SMILES: CNCCc1cc(OC)c(Nc2ncc(Cl)c(Nc3ccccc3S(=O)(=O)C(C)C)n2)cc1C ; O=C(O)CCCCCC#Cc1cccc2c1C(=O)N(C1CCC(=O)NC1=O)C2=O ; COc1cc(CCN(C)C(=O)CCCCCC#Cc2cccc3c2C(=O)N(C2CCC(=O)NC2=O)C3=O)c(C)cc1Nc1ncc(Cl)c(Nc2ccccc2S(=O)(=O)C(C)C)n1]

Compound 10 was synthesized based on similar procedure as compound 1. ¹H NMR (400 MHz, DMSO-d₆): δ 11.13 (s, 1H), 9.47 (brs, 1H), 8.54-8.44 (m, 1H), 8.31-8.19 (m, 2H), 7.88-7.76 (m, 4H), 7.67-7.57 (m, 1H), 7.55-7.45 (m, 1H), 7.36-7.30 (m, 1H), 6.81 (d, J=3.6 Hz, 0.8H), 6.72 (s, 0.2H), 5.14 (dd, J=5.6 Hz, 12.8 Hz, 1H), 3.77-3.70 (m, 3H), 3.51-3.39 (m, 3H), 2.96 (s, 1H), 2.94-2.83 (m, 3H), 2.80 (t, J=6.8 Hz, 1H), 2.73-2.66 (m, 1H), 2.64-2.54 (m, 1H), 2.47-2.42 (m, 1H), 2.41-2.27 (m, 2H), 2.15 (s, 2H), 2.12-2.00 (m, 3H), 1.66-1.40 (m, 4H), 1.39-1.22 (m, 1H), 1.15 (dd, J=2.8 Hz, 6.8 Hz, 6H). ESI-MS (EI⁺, m/z): 882.3.

### Example 12: Synthesis of N-(4-((5-chloro-4-((2-(isopropylsulfonyl)phenyl)amino)pyrimidin-2-yl)amino)-5-methoxy-2-methylphenethyl)-3-(2-(2-((3-(2-(2,6-dioxopiperidin-3-yl)-1,3-dioxoisoindolin-4-yl)prop-2-yn-1-yl)oxy)ethoxy)ethoxy)-N-methylpropanamide (11)

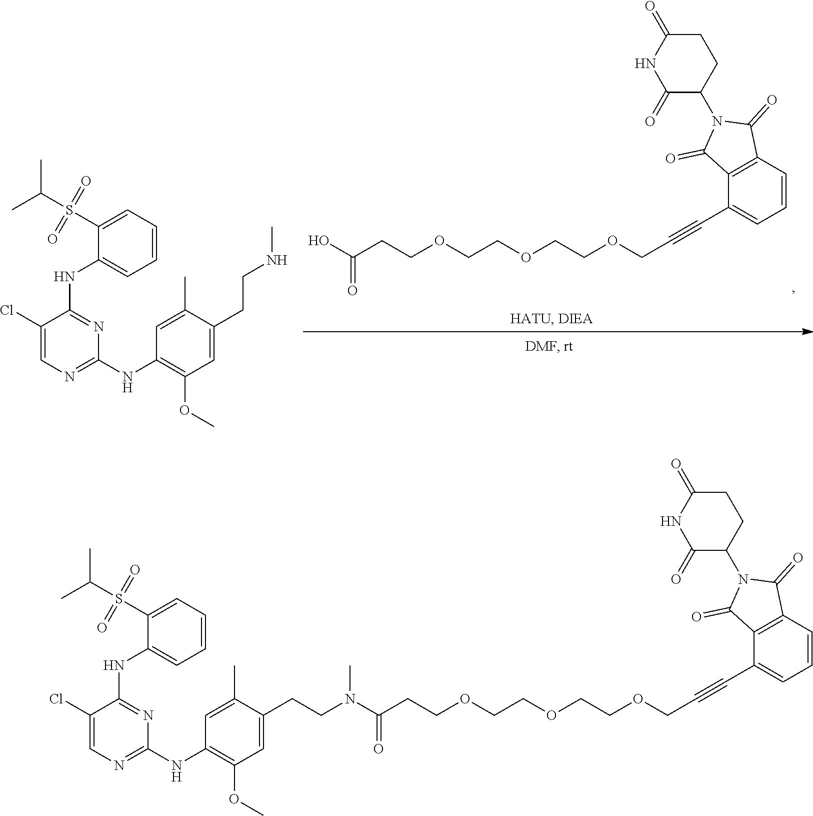 [SMILES: CNCCc1cc(OC)c(Nc2ncc(Cl)c(Nc3ccccc3S(=O)(=O)C(C)C)n2)cc1C ; O=C(O)CCOCCOCCOCC#Cc1cccc2c1C(=O)N(C1CCC(=O)NC1=O)C2=O ; COc1cc(CCN(C)C(=O)CCOCCOCCOCC#Cc2cccc3c2C(=O)N(C2CCC(=O)NC2=O)C3=O)c(C)cc1Nc1ncc(Cl)c(Nc2ccccc2S(=O)(=O)C(C)C)n1]

Compound 11 was synthesized based on similar procedure as compound 1. ¹H NMR (400 MHz, DMSO-d₆): δ 11.15 (s, 1H), 9.47 (s, 1H), 8.53-8.46 (m, 1H), 8.25-8.20 (m, 2H), 7.94-7.87 (m, 3H), 7.84-7.79 (m, 1H), 7.67-7.59 (m, 1H), 7.52-7.44 (m, 1H), 7.38-7.31 (m, 1H), 6.96-6.81 (m, 1H), 5.16 (dd, J=5.6 Hz, 13.2 Hz, 1H), 4.48-4.41 (m, 2H), 3.76 (s, 3H), 3.68-3.38 (m, 13H), 2.97-2.52 (m, 9H), 2.40 (t, J=6.8 Hz, 3H), 2.19-2.11 (m, 3H), 2.10-2.00 (m, 1H), 1.15 (d, J=6.8 Hz, 6H). ESI-MS (EI⁺, m/z): 958.1.

### Example 13: General Procedure for Reductive Amination

A solution of aldehyde (0.1 mmol) and amine hydrochloride (0.1 mmol) in 2.0 mL of MeOH and THE (v/v, 2:1) was stirred at rt under N₂ for 30 minutes. NaBH₃CN (0.3 mmol) was added at 0° C. in portions, and the reaction mixture was stirred at rt for 16 hours. The reaction was monitored by LCMS. The mixture was partitioned between ethyl acetate and water, and the combined organic phases were washed with water, brine, dried over Na₂SO₄ and concentrated in vacuo. The crude product was purified by silica gel column (SGC) or prep-high-performance liquid chromatography (HPLC).

### Example 14: Synthesis of 4-(7-((4-((5-chloro-4-((2-(dimethylphosphoryl)phenyl)amino)pyrimidin-2-yl)amino)-5-methoxy-2-methylphenethyl)amino)hept-1-yn-1-yl)-2-(2,6-dioxopiperidin-3-yl)isoindoline-1,3-dione (19)

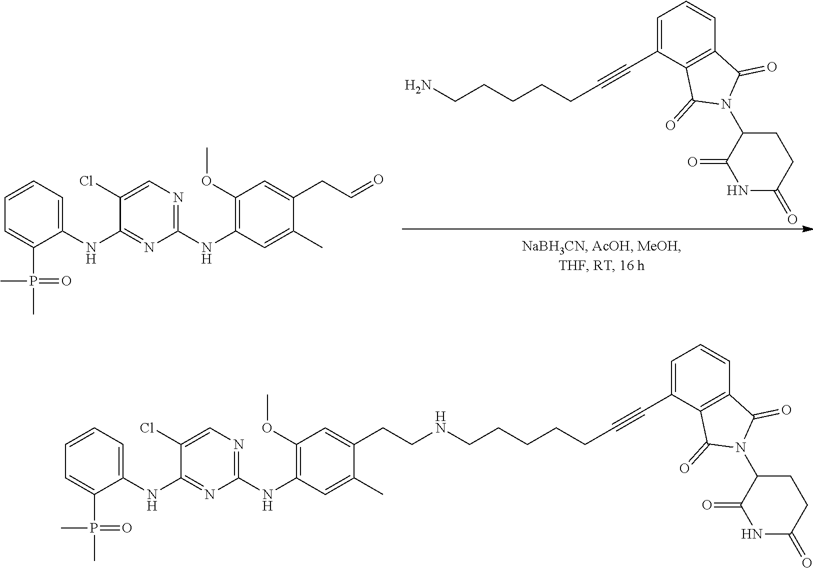 [SMILES: COc1cc(CC=O)c(C)cc1Nc1ncc(Cl)c(Nc2ccccc2P(C)(C)=O)n1 ; NCCCCCC#Cc1cccc2c1C(=O)N(C1CCC(=O)NC1=O)C2=O ; COc1cc(CCNCCCCCC#Cc2cccc3c2C(=O)N(C2CCC(=O)NC2=O)C3=O)c(C)cc1Nc1ncc(Cl)c(Nc2ccccc2P(C)(C)=O)n1]

Following general procedure for reductive amination (Example 13) afforded the title compound as a white powder (25.4 mg, 12.6%). ¹H NMR (400 MHz, DMSO-d₆): δ 11.11 (s, 1H), 8.47-8.44 (m, 1H), 8.29 (s, 1H), 8.15-8.14 (m, 1H), 8.06-8.04 (m, 1H), 7.90-7.82 (m, 3H), 7.60-7.54 (m, 2H), 7.42-7.38 (m, 1H), 7.15-7.10 (m, 1H), 6.86 (s, 1H), 5.17-5.13 (m, 1H), 2.71-2.67 (m, 6H), 2.98-2.84 (m, 5H), 2.61-2.52 (m, 4H), 2.20-2.04 (m, 4H), 1.79-1.48 (m, 12H). ESI-MS (EI⁺, m/z): 810.3.

### Example 15: Synthesis of (S)-7-(2-(2-((4-((5-chloro-4-((2-(isopropylsulfonyl)phenyl)amino)pyrimidin-2-yl)amino)-5-methoxy-2-methylphenethyl)amino)ethoxy)ethoxy)-2-((S)-3,3-dimethyl-2-((S)-2-(methylamino)propanamido)butanoyl)-N-((S)-1,2,3,4-tetrahydronaphthalen-1-yl)-1,2,3,4-tetrahydroisoquinoline-3-carboxamide (37)

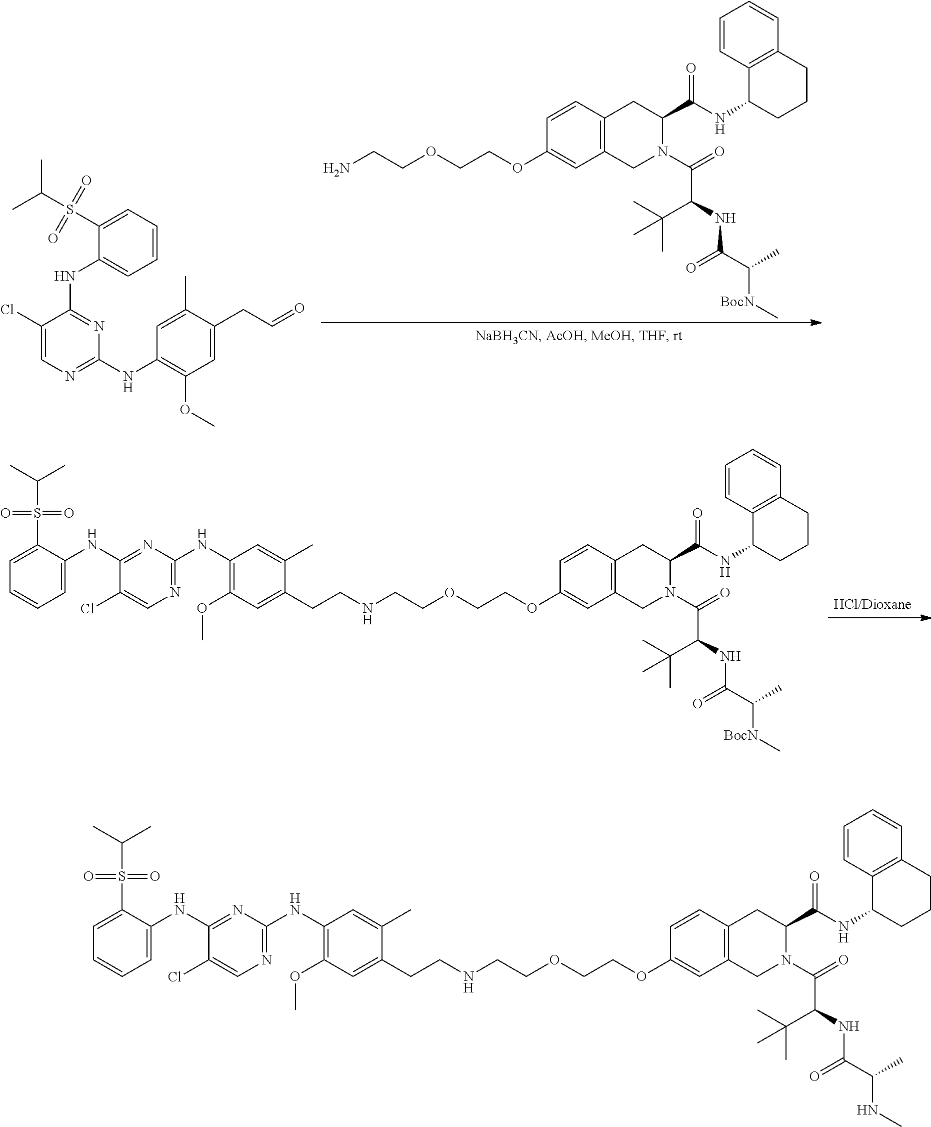

Following general procedure for reductive amination (Example 13) afforded tert-butyl ((S)-1-(((S)-1-((S)-7-(2-(2-((4-((5-chloro-4-((2-(isopropylsulfonyl)phenyl)amino)pyrimidin-2-yl)amino)-5-methoxy-2-methylphenethyl)amino)ethoxy)ethoxy)-3-(((S)-1,2,3,4-tetrahydronaphthalen-1-yl)carbamoyl)-3,4-dihydroisoquinolin-2(1H)-yl)-3,3-dimethyl-1-oxobutan-2-yl)amino)-1-oxopropan-2-yl)(methyl)carbamate as a yellow powder (30 mg, 36.3%). ESI-MS (EI⁺, m/z): 590.80.

A solution of tert-butyl ((S)-1-(((S)-1-((S)-7-(2-(2-((4-((5-chloro-4-((2-(isopropylsulfonyl)phenyl)amino)pyrimidin-2-yl)amino)-5-methoxy-2-methylphenethyl)amino)ethoxy)ethoxy)-3-(((S)-1,2,3,4-tetrahydronaphthalen-1-yl)carbamoyl)-3,4-dihydroisoquinolin-2(1H)-yl)-3,3-dimethyl-1-oxobutan-2-yl)amino)-1-oxopropan-2-yl)(methyl)carbamate (24.0 mg, 0.02 mmol) in HCl/dioxane (4 M, 0.5 mL) stirred at rt for 3 hours. LCMS showed the reaction was completed. The mixture was concentrated to dryness in vacuo and afford the title product as a white solid (16 mg, 100%). ¹H NMR (400 MHz, DMSO-d₆) δ 9.73 (s, 1H), 9.59-9.47 (m, 1H), 9.20-9.32 (m, 2H), 8.96-8.86 (m, 1H), 8.76-8.63 (m, 2H), 8.34 (d, J=10.6 Hz, 2H), 8.24 (d, J=8.6 Hz, 1H), 7.86 (d, J=7.9 Hz, 1H), 7.67 (t, J=7.6 Hz, 1H), 7.40-7.43 (m, 2H), 7.13-7.06 (m, 3H), 7.03 (d, J=7.2 Hz, 1H), 6.92 (d, J=2.1 Hz, 1H), 6.87 (s, 1H), 6.79 (d, J=8.5 Hz, 1H), 4.99 (d, J=8.9 Hz, 1H), 4.90-4.73 (m, 2H), 4.67 (d, J=17.0 Hz, 2H), 4.12 (s, 3H), 3.94 (s, 2H), 3.76 (s, 6H), 3.48-3.43 (m, 1H), 3.18 (s, 2H), 3.08 (s, 2H), 2.97 (d, J=6.3 Hz, 3H), 2.78-2.62 (m, 2H), 2.44 (t, J=5.1 Hz, 3H), 2.10 (s, 3H), 1.31-1.23 (m, 4H), 1.14 (d, J=6.8 Hz, 6H), 1.08 (s, 7H), 0.98 (d, J=3.1 Hz, 3H). ESI-MS (EI⁺, m/z): 540.80.

### Example 16: Synthesis of (S)-7-(2-(2-(2-((4-((5-chloro-4-((2-(isopropylsulfonyl)phenyl)amino)pyrimidin-2-yl)amino)-5-methoxy-2-methylphenethyl)amino)ethoxy)ethoxy)ethoxy)-2-((S)-3,3-dimethyl-2-((S)-2-(methylamino)propanamido)butanoyl)-N-((R)-1,2,3,4-tetrahydronaphthalen-1-yl)-1,2,3,4-tetrahydroisoquinoline-3-carboxamide (38)

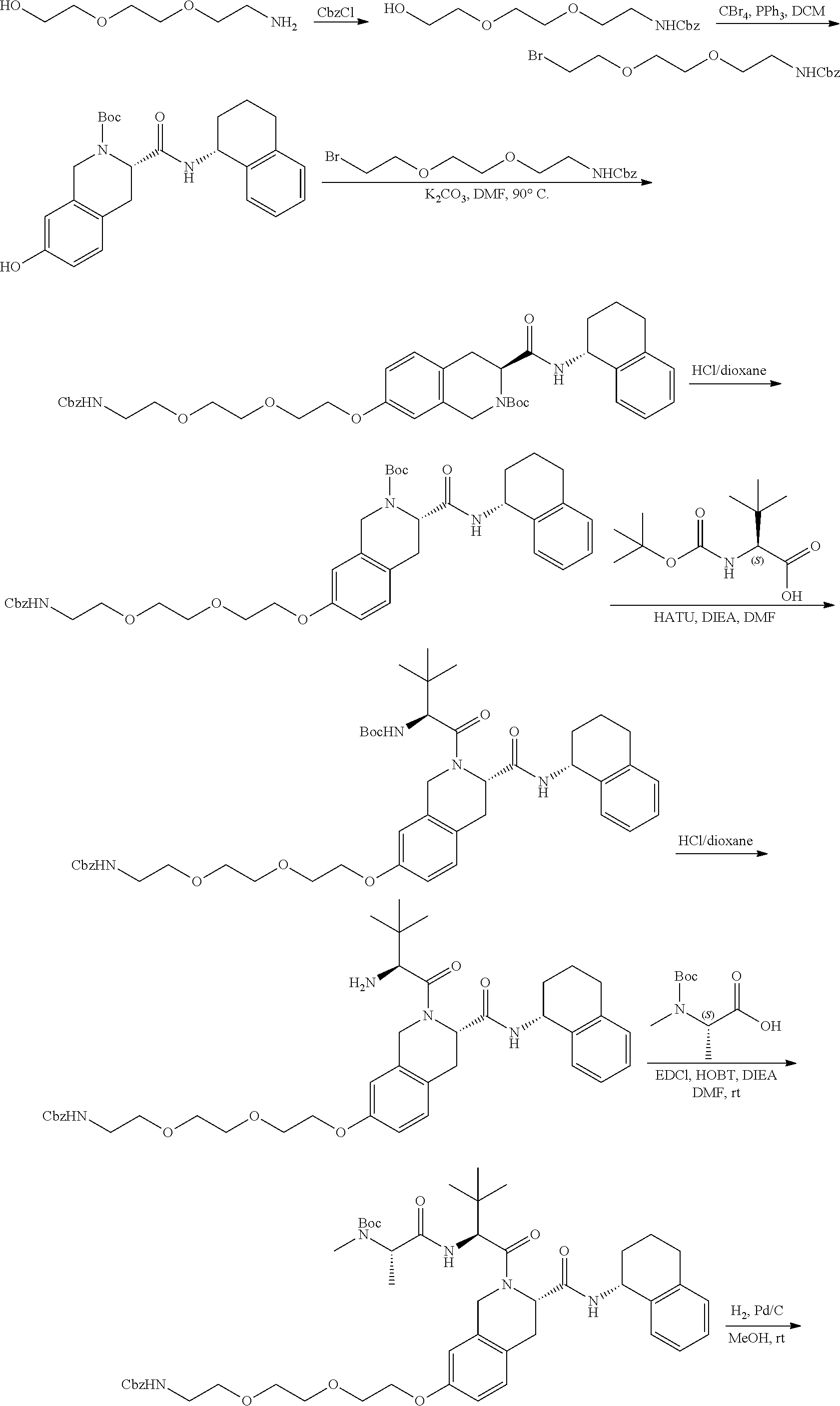

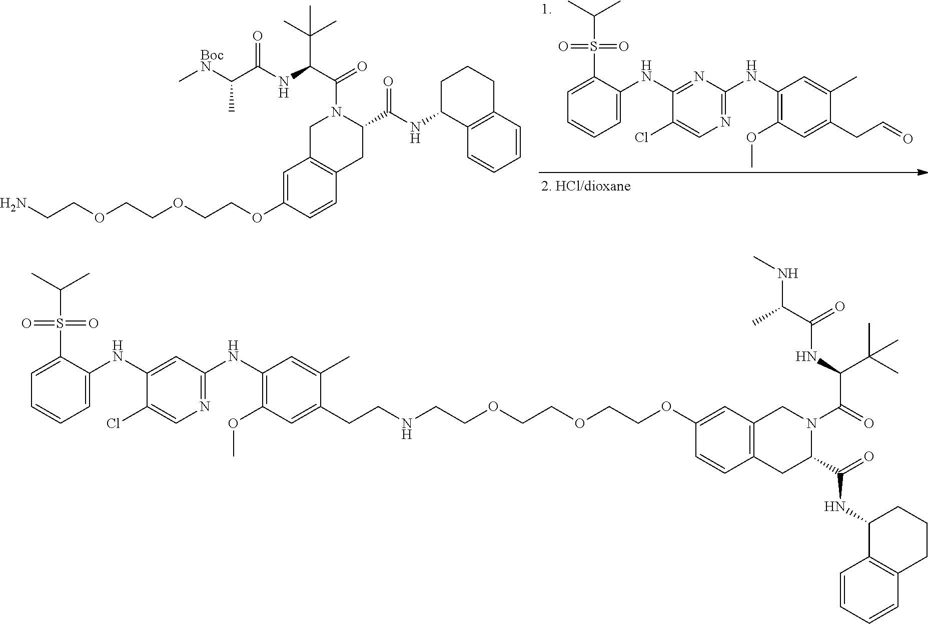

Following general procedure for reductive amination (Example 13) afforded title compound as a white powder (9 mg, 6.33%). ¹H NMR (400 MHz, DMSO-d₆) δ 9.61 (s, 1H), 9.33 (s, 1H), 9.19-8.98 (m, 2H), 8.86 (s, 1H), 8.65 (d, J=8.8 Hz, 1H), 8.54-8.39 (m, 2H), 8.25 (d, J=22.4 Hz, 2H), 7.85 (d, J=7.9 Hz, 1H), 7.65 (t, J=8.6 Hz, 1H), 7.47 (s, 1H), 7.39 (t, J=7.9 Hz, 1H), 7.15-6.98 (m, 4H), 6.94-6.73 (m, 3H), 5.09-4.96 (m, 1H), 4.91-4.72 (m, 2H), 4.70-4.62 (m, 2H), 4.59-4.43 (m, 1H), 4.32-4.26 (m, 1H), 4.07-4.02 (m, 2H), 3.78-3.73 (m, 7H), 3.68-3.60 (m, 6H), 3.39 (s, 3H), 3.16 (s, 1H), 3.12-2.90 (m, 6H), 2.70 (s, 2H), 2.45 (t, J=5.2 Hz, 3H), 2.12 (s, 3H), 1.23 (s, 4H), 1.15 (d, J=6.8 Hz, 6H), 1.08 (s, 6H), 0.98 (d, J=3.4 Hz, 3H). ESI-MS (EI⁺, m/z): 562.90.

### Example 17: Synthesis of N¹-(4-((5-chloro-4-((2-(isopropylsulfonyl)phenyl)amino)pyrimidin-2-yl)amino)-5-methoxy-2-methylphenethyl)-N⁸-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-N¹-methyloctanediamide (40)

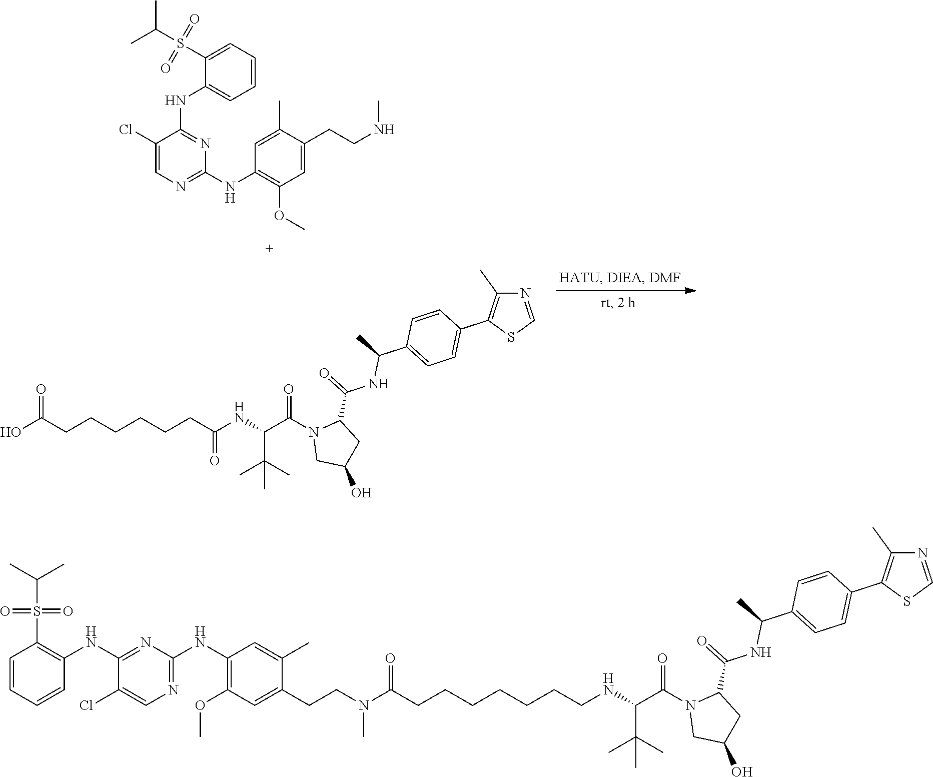

Compound 40 was synthesized based on similar procedure as compound 1. ¹H NMR (400 MHz, DMSO-d₆) δ 9.52 (s, 1H), 8.97 (s, 1H), 8.46 (t, J=7.0 Hz, 1H), 8.36 (d, J=9.2 Hz, 1H), 8.29 (d, J=11.8 Hz, 1H), 8.24 (d, J=2.6 Hz, 1H), 7.82 (d, J=8.0 Hz, 1H), 7.76 (dd, J=14.6, 9.3 Hz, 1H), 7.64 (q, J=7.5 Hz, 1H), 7.50-7.33 (m, 6H), 6.83 (d, J=4.0 Hz, 1H), 4.95-4.87 (m, 1H), 4.53-4.46 (m, 1H), 4.45-4.37 (m, 1H), 4.27 (s, 1H), 3.75 (s, 3H), 3.59 (s, 2H), 3.44 (s, 2H), 2.93 (s, 2H), 2.84 (s, 1H), 2.80-2.75 (m, 1H), 2.71-2.66 (m, 1H), 2.44 (s, 3H), 2.24 (t, J=7.4 Hz, 2H), 2.14 (d, J=6.3 Hz, 3H), 2.08-1.96 (m, 3H), 1.78 (t, J=10.6 Hz, 1H), 1.50-1.41 (m, 3H), 1.36 (d, J=6.9 Hz, 4H), 1.22 (s, 5H), 1.14 (d, J=6.7 Hz, 8H), 0.91 (d, J=6.9 Hz, 9H). ESI-MS (EI⁺, m/2z): 543.8.

### Example 18: Synthesis of N¹-(4-((5-chloro-4-((2-(isopropylsulfonyl)phenyl)amino)pyrimidin-2-yl)amino)-5-methoxy-2-methylphenethyl)-N⁹-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-N¹-methylnonanediamide (41)

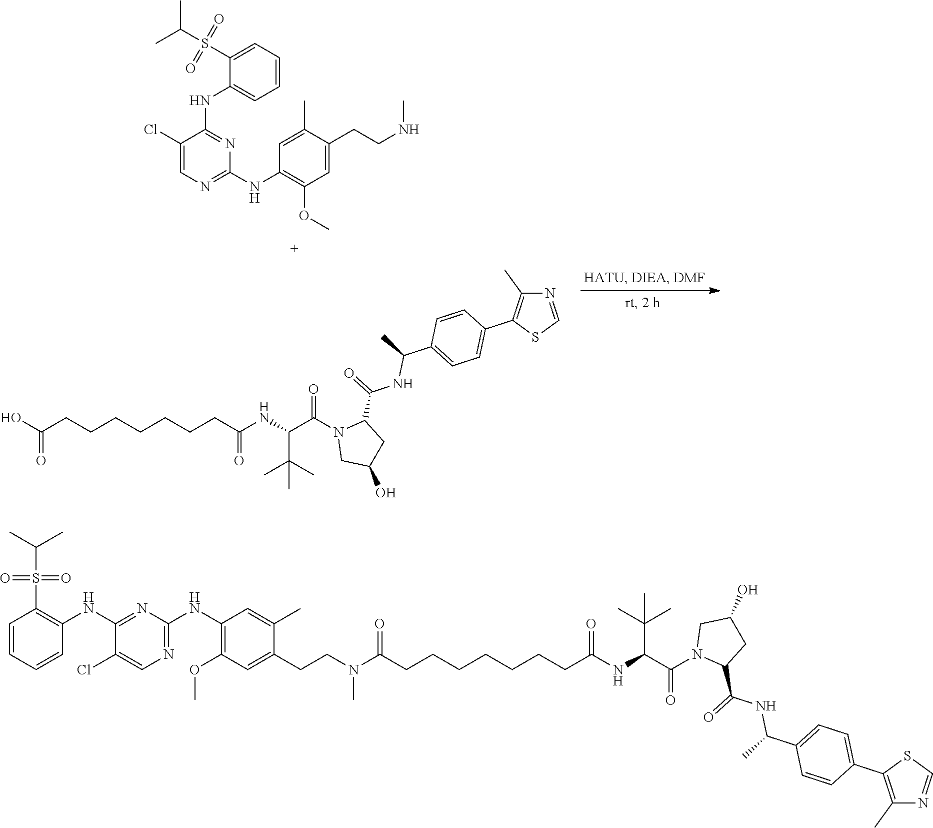

Compound 41 was synthesized based on similar procedure as compound 1. ¹H NMR (400 MHz, DMSO-d₆) δ 9.53 (s, 1H), 8.99 (s, 1H), 8.49 (s, 1H), 8.37 (d, J=7.4 Hz, 1H), 8.33-8.24 (m, 2H), 7.81 (dd, J=23.9, 9.8 Hz, 2H), 7.65 (d, J=6.1 Hz, 1H), 7.49-7.41 (m, 3H), 7.41-7.32 (m, 3H), 6.84 (d, J=5.4 Hz, 1H), 4.96-4.88 (m, 1H), 4.52 (dd, J=9.2, 3.9 Hz, 1H), 4.42 (t, J=7.7 Hz, 1H), 4.29 (s, 1H), 3.77 (s, 3H), 3.61 (s, 2H), 3.47 (d, J=7.3 Hz, 2H), 2.95 (s, 2H), 2.86 (s, 2H), 2.82-2.76 (m, 1H), 2.75-2.66 (m, 2H), 2.46 (s, 3H), 2.35-2.19 (m, 3H), 2.16 (d, J=6.7 Hz, 3H), 2.12-1.97 (m, 3H), 1.82-1.75 (m, 1H), 1.47 (d, J=8.3 Hz, 3H), 1.38 (d, J=7.0 Hz, 3H), 1.32 (d, J=5.3 Hz, 1H), 1.25 (d, J=6.2 Hz, 4H), 1.16 (d, J=6.7 Hz, 8H), 0.93 (d, J=5.2 Hz, 9H). ESI-MS (EI⁺, m/2z): 550.9.

### Example 19: Synthesis of N¹-(4-((5-chloro-4-((2-(isopropylsulfonyl)phenyl)amino)pyrimidin-2-yl)amino)-5-methoxy-2-methylphenethyl)-N¹⁰-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-N¹-methyldecanediamide (42)

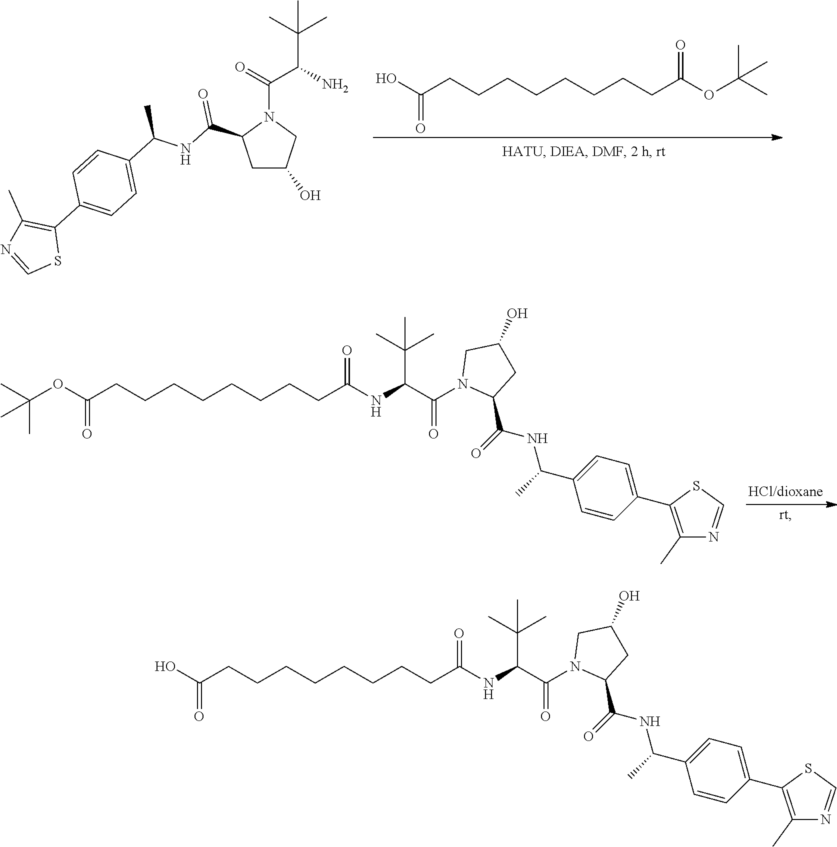 [SMILES: Cc1ncsc1-c1ccc([C@@H](C)NC(=O)[C@@H]2C[C@@H](O)CN2C(=O)[C@@H](N)C(C)(C)C)cc1 ; CC(C)(C)OC(=O)CCCCCCCCC(=O)O ; Cc1ncsc1-c1ccc([C@H](C)NC(=O)[C@@H]2C[C@@H](O)CN2C(=O)[C@@H](NC(=O)CCCCCCCCC(=O)OC(C)(C)C)C(C)(C)C)cc1 ; Cc1ncsc1-c1ccc([C@H](C)NC(=O)[C@@H]2C[C@@H](O)CN2C(=O)[C@@H](NC(=O)CCCCCCCCC(=O)O)C(C)(C)C)cc1]

tert-Butyl 10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecanoate was synthesized based on similar procedure as compound 1.

HCl in dioxane (1 mL) was added to solution of tert-butyl 10-(((S)-1-((2S,4R)-4-hydroxy-2-(((S)-1-(4-(4-methylthiazol-5-yl)phenyl)ethyl)carbamoyl)pyrrolidin-1-yl)-3, 3-dimethyl-1-oxobutan-2-yl)amino)-10-oxodecanoate (48 mg, 0.07 mmol) in DCM (1 mL). The resulting mixture stirred at rt for 2 hours. After TLC showed reaction completion, the reaction mixture was concentrated in vacuo to afford 10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecanoic acid as a yellow solid (50 mg).

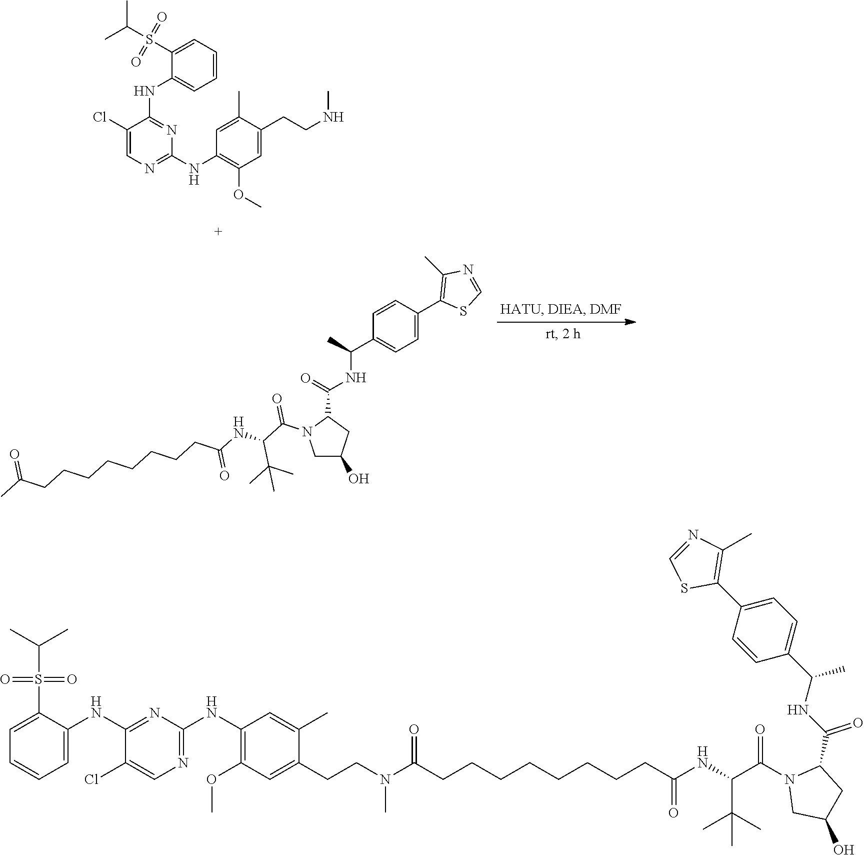

Compound 42 was synthesized based on similar procedure as compound 1. ¹H NMR (400 MHz, DMSO-d₆) δ 9.56 (s, 1H), 8.98 (s, 1H), 8.47 (s, 1H), 8.37 (d, J=8.0 Hz, 2H), 8.26 (d, J=3.8 Hz, 1H), 7.79 (d, J=28.4 Hz, 2H), 7.65 (s, 1H), 7.55-7.26 (m, 6H), 6.83 (d, J=8.5 Hz, 1H), 4.95-4.86 (m, 1H), 4.51 (dd, J=9.3, 3.2 Hz, 1H), 4.41 (t, J=8.2 Hz, 1H), 4.27 (s, 1H), 3.76 (s, 3H), 3.60 (s, 2H), 2.94 (s, 2H), 2.85 (s, 2H), 2.82-2.75 (m, 1H), 2.75-2.64 (m, 2H), 2.45 (s, 3H), 2.34-2.17 (m, 3H), 2.14 (d, J=6.8 Hz, 3H), 2.10-1.96 (m, 3H), 1.83-1.73 (m, 1H), 1.46 (d, J=7.3 Hz, 3H), 1.37 (d, J=7.0 Hz, 3H), 1.24 (s, 6H), 1.15 (d, J=5.6 Hz, 9H), 0.92 (d, J=4.3 Hz, 8H). ESI-MS (EI⁺, m/2z): 557.8.

### Example 20: Synthesis of 4-(7-((4-((5-chloro-4-((2-(isopropylsulfonyl)phenyl)amino)pyrimidin-2-yl)amino)-5-methoxy-2-methylphenethyl)amino)hept-1-yn-1-yl)-2-(2,6-dioxopiperidin-3-yl)isoindoline-1,3-dione (49)

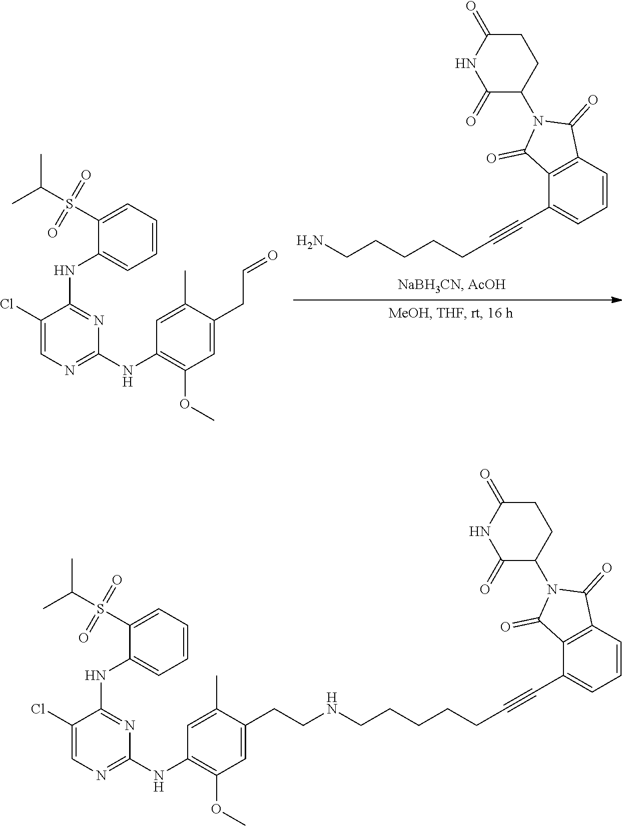 [SMILES: COc1cc(CC=O)c(C)cc1Nc1ncc(Cl)c(Nc2ccccc2S(=O)(=O)C(C)C)n1 ; NCCCCCC#Cc1cccc2c1C(=O)N(C1CCC(=O)NC1=O)C2=O ; COc1cc(CCNCCCCCC#Cc2cccc3c2C(=O)N(C2CCC(=O)NC2=O)C3=O)c(C)cc1Nc1ncc(Cl)c(Nc2ccccc2S(=O)(=O)C(C)C)n1]

Following general procedure for reductive amination (Example 13) afforded the title compound as a white powder (12.5 mg, 6.0%). ¹H NMR (400 MHz, DMSO-d₆): δ 9.48 (brs, 1H), 8.51-8.49 (m, 1H), 8.33-8.23 (m, 3H), 7.90-7.81 (m, 4H), 7.62 (t, J=8.0 Hz, 1H), 7.46 (s, 1H), 7.33 (t, J=8.0 Hz, 1H), 6.86 (s, 1H), 5.17-5.13 (m, 1H), 3.76 (s, 4H), 2.86-2.76 (m, 8H), 2.62-2.57 (m, 2H), 2.14 (s, 3H), 2.07-2.03 (m, 1H), 1.62-1.46 (m, 6H), 1.16 (d, J=4.0 Hz, 6H). ESI-MS (EI⁺, m/z): 840.3.

### Example 21: Synthesis of (2S,4R)-1-((S)-2-(3-(3-((4-((5-chloro-4-((2-(isopropylsulfonyl)phenyl)amino)pyrimidin-2-yl)amino)-5-methoxy-2-methylphenethyl)(methyl)amino)-3-oxopropoxy)propanamido)-3,3-dimethylbutanoyl)-4-hydroxy-N-((S)-1-(4-(4-methylthiazol-5-yl)phenyl)ethyl)pyrrolidine-2-carboxamide (52)

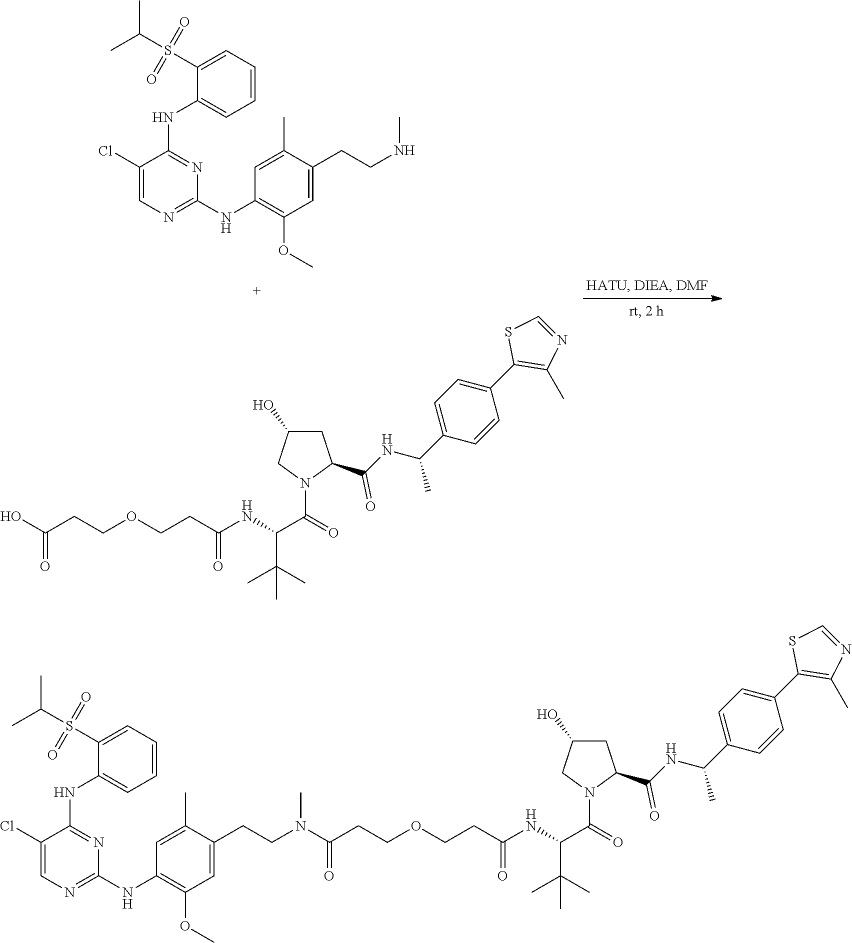

Compound 52 was synthesized based on similar procedure as compound 1. ¹H NMR (400 MHz, DMSO-d₆) δ 9.45 (s, 1H), 8.98 (s, 1H), 8.50 (d, J=7.2 Hz, 1H), 8.40-8.34 (m, 1H), 8.24 (d, J=2.7 Hz, 2H), 7.85 (dd, J=18.5, 8.7 Hz, 2H), 7.63 (q, J=7.1 Hz, 1H), 7.49 (d, J=16.2 Hz, 1H), 7.42 (dd, J=8.2, 2.4 Hz, 2H), 7.39-7.32 (m, 3H), 6.85 (d, J=5.2 Hz, 1H), 5.13 (s, 1H), 4.90 (q, J=7.1 Hz, 1H), 4.52 (t, J=8.6 Hz, 1H), 4.42 (t, J=7.9 Hz, 1H), 4.28 (s, 1H), 3.77 (s, 3H), 3.64-3.52 (m, 6H), 3.43 (s, 2H), 2.96 (s, 2H), 2.84 (s, 1H), 2.82-2.76 (m, 1H), 2.73-2.68 (m, 1H), 2.33 (ddt, J=20.7, 15.6, 7.6 Hz, 2H), 2.16 (d, J=5.7 Hz, 3H), 2.00 (dt, J=13.8, 5.7 Hz, 2H), 1.78 (td, J=8.6, 4.4 Hz, 1H), 1.48-1.42 (m, 1H), 1.36 (t, J=7.2 Hz, 3H), 1.23 (s, 3H), 1.15 (d, J=6.8 Hz, 6H), 0.90 (s, 9H). ESI-MS (EI⁺, m/2z): 537.8.

### Example 22: Synthesis of N¹-(4-((5-chloro-4-((2-(dimethylphosphoryl)phenyl)amino)pyrimidin-2-yl)amino)-5-methoxy-2-methylphenethyl)-N¹⁰-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-N-methyldecanediamide (68)

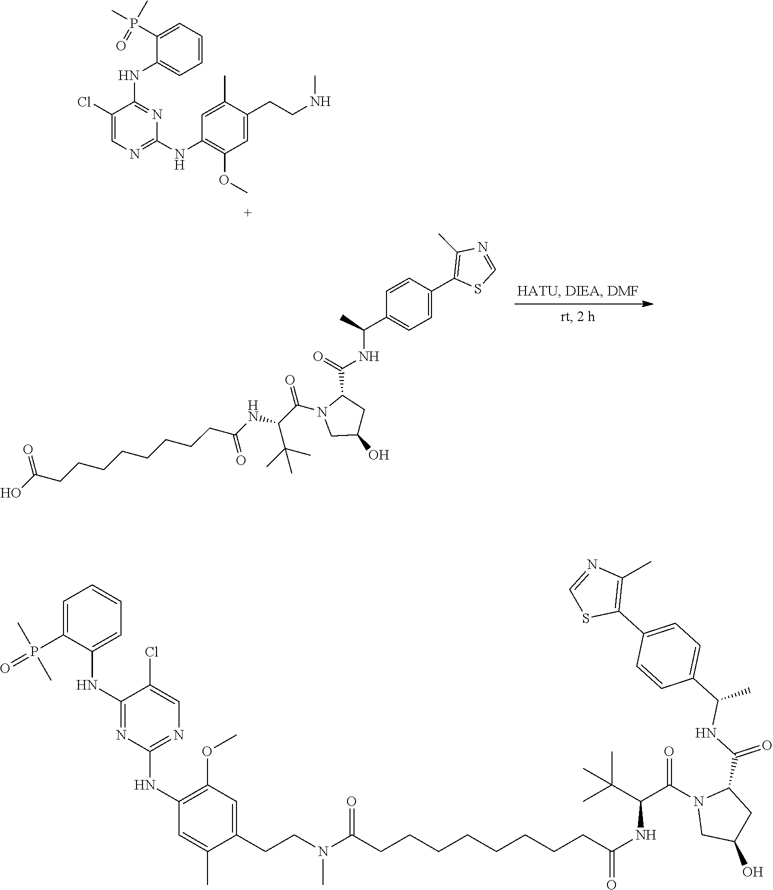 [SMILES: CNCCc1cc(OC)c(Nc2ncc(Cl)c(Nc3ccccc3P(C)(C)=O)n2)cc1C ; Cc1ncsc1-c1ccc([C@H](C)NC(=O)[C@@H]2C[C@@H](O)CN2C(=O)[C@@H](NC(=O)CCCCCCCCC(=O)O)C(C)(C)C)cc1 ; COc1cc(CCN(C)C(=O)CCCCCCCCC(=O)N[C@H](C(=O)N2C[C@H](O)C[C@H]2C(=O)N[C@@H](C)c2ccc(-c3scnc3C)cc2)C(C)(C)C)c(C)cc1Nc1ncc(Cl)c(Nc2ccccc2P(C)(C)=O)n1]

Compound 68 was synthesized based on similar procedure as compound 1. ¹H NMR (400 MHz, DMSO-d₆) δ 11.44 (d, J=23.6 Hz, 1H), 8.98 (s, 1H), 8.58-8.33 (m, 3H), 8.19 (s, 1H), 7.78 (d, J=12.3 Hz, 1H), 7.64-7.55 (m, 1H), 7.48 (s, 1H), 7.46-7.35 (m, 5H), 7.18 (s, 1H), 6.86 (d, J=13.1 Hz, 1H), 4.91 (d, J=14.4 Hz, 1H), 4.50 (d, J=9.5 Hz, 1H), 4.41 (s, 1H), 4.27 (s, 1H), 3.78 (s, 3H), 2.96 (s, 2H), 2.85 (s, 1H), 2.78 (s, 1H), 2.72 (s, 1H), 2.45 (s, 3H), 2.23 (dd, J=31.5, 8.0 Hz, 5H), 2.08 (s, 1H), 2.00 (d, J=10.0 Hz, 2H), 1.78 (d, J=13.6 Hz, 6H), 1.46 (s, 3H), 1.35 (s, 3H), 1.23 (d, J=14.2 Hz, 6H), 1.12 (d, J=14.2 Hz, 4H), 0.91 (s, 9H). ESI-MS (EI⁺, m/2z): 542.9.

### Example 23: Synthesis of N¹-(4-((5-chloro-4-((2-(dimethylphosphoryl)phenyl)amino)pyrimidin-2-yl)amino)-5-methoxy-2-methylphenethyl)-N⁹-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-N¹-methylnonanediamide (69)

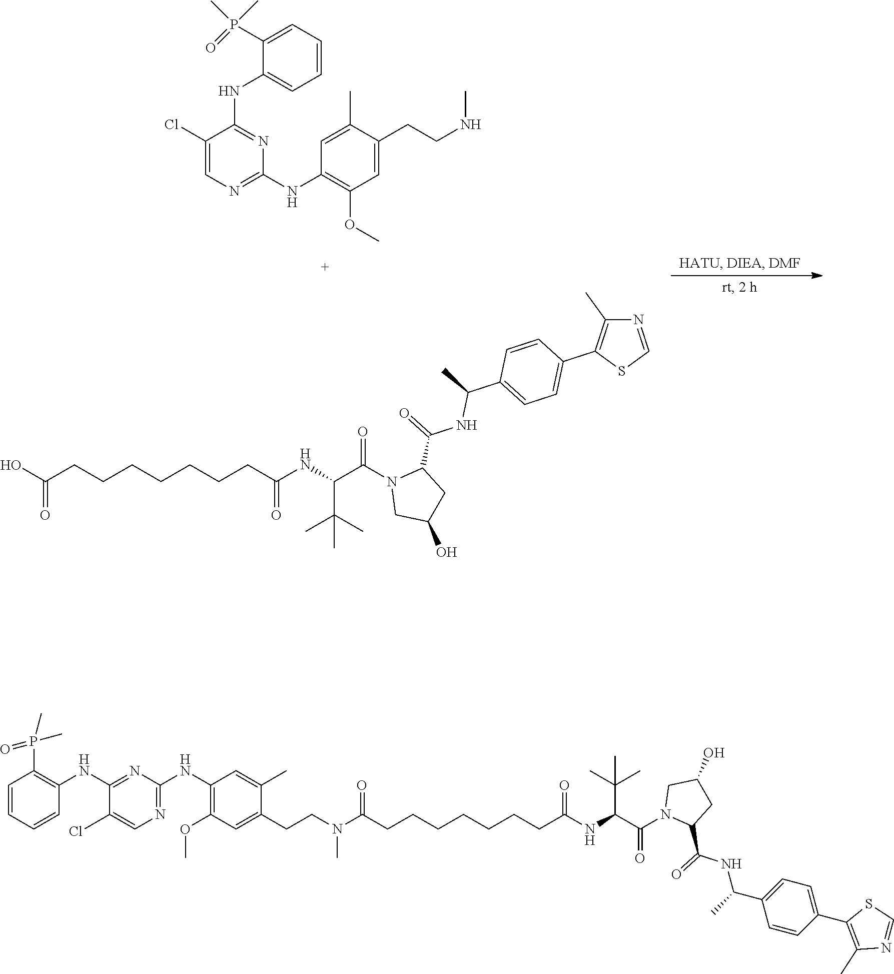

Compound 69 was synthesized based on similar procedure as compound 1. ¹H NMR (400 MHz, DMSO-d₆) δ 11.26 (s, 2H), 8.97 (s, 1H), 8.44 (d, J=8.4 Hz, 1H), 8.36 (d, J=7.2 Hz, 1H), 8.16 (s, 1H), 7.76 (t, J=9.6 Hz, 1H), 7.57 (dd, J=23.6, 10.7 Hz, 2H), 7.46-7.34 (m, 4H), 7.15 (t, J=7.0 Hz, 1H), 6.83 (d, J=7.2 Hz, 1H), 4.92 (d, J=7.7 Hz, 1H), 4.51 (dd, J=10.2, 3.7 Hz, 1H), 4.41 (t, J=7.8 Hz, 1H), 4.27 (s, 1H), 3.77 (s, 3H), 3.59 (s, 2H), 2.95 (s, 2H), 2.85 (s, 1H), 2.81-2.75 (m, 1H), 2.75-2.65 (m, 2H), 2.44 (s, 3H), 2.33 (s, 1H), 2.29-2.23 (m, 2H), 2.18 (d, J=8.0 Hz, 3H), 2.14-1.94 (m, 4H), 1.77 (d, J=13.5 Hz, 6H), 1.46 (d, J=11.0 Hz, 3H), 1.36 (d, J=7.0 Hz, 3H), 1.25 (dd, J=10.3, 4.7 Hz, 5H), 1.14 (s, 3H), 0.92 (d, J=4.9 Hz, 9H). ESI-MS (EI⁺, m/2z): 535.8.

### Example 24: Synthesis of N¹-(4-((5-chloro-4-((2-(dimethylphosphoryl)phenyl)amino)pyrimidin-2-yl)amino)-5-methoxy-2-methylphenethyl)-N⁸-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-N′-methyloctanediamide (70)

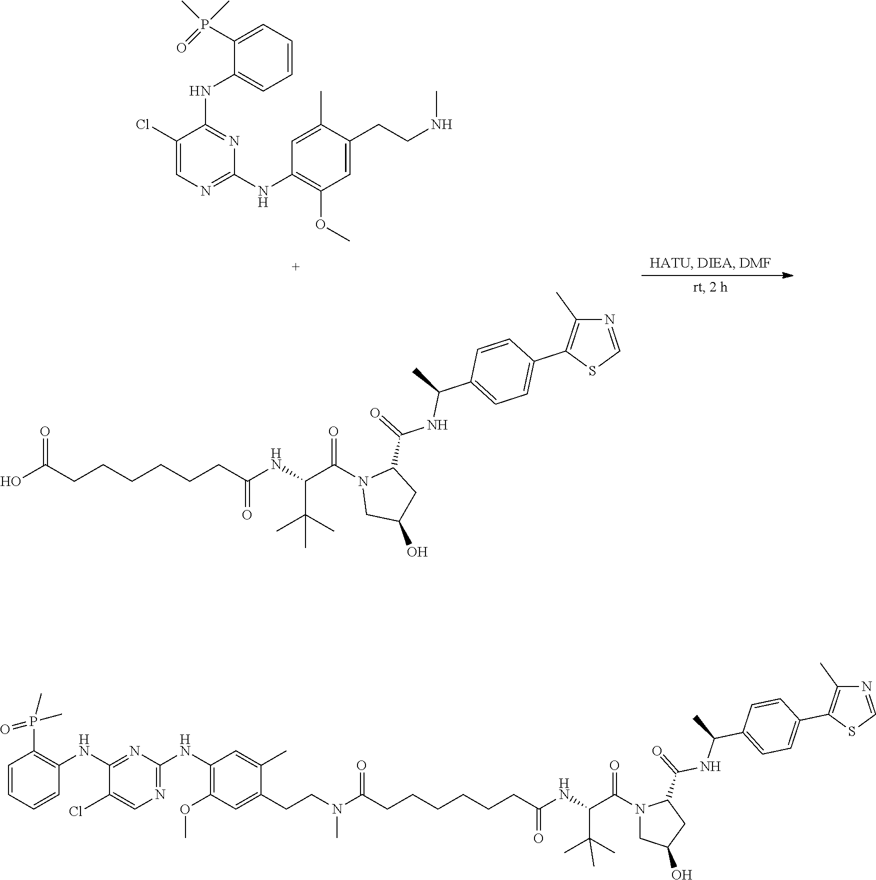

Compound 70 was synthesized based on similar procedure as compound 1. ¹H NMR (400 MHz, DMSO-d₆) δ 11.15 (s, 1H), 8.98 (s, 1H), 8.46 (d, J=8.5 Hz, 1H), 8.37 (d, J=7.3 Hz, 1H), 8.14 (d, J=3.2 Hz, 1H), 8.02 (d, J=6.8 Hz, 1H), 7.82-7.72 (m, 1H), 7.57 (d, J=19.3 Hz, 2H), 7.46-7.36 (m, 4H), 7.14 (t, J=8.1 Hz, 1H), 6.83 (d, J=2.7 Hz, 1H), 5.10 (s, 1H), 4.95-4.86 (m, 1H), 4.56-4.48 (m, 1H), 4.42 (t, J=8.6 Hz, 1H), 4.27 (s, 1H), 3.78 (s, 3H), 3.60 (s, 2H), 3.47-3.41 (m, 2H), 2.94 (d, J=9.4 Hz, 2H), 2.86 (s, 2H), 2.82-2.76 (m, 1H), 2.70 (dd, J=15.0, 7.6 Hz, 2H), 2.44 (d, J=5.1 Hz, 3H), 2.33-2.22 (m, 2H), 2.19 (d, J=7.2 Hz, 3H), 2.13-1.96 (m, 4H), 1.77 (d, J=13.5 Hz, 6H), 1.47 (d, J=9.4 Hz, 3H), 1.37 (d, J=7.0 Hz, 3H), 1.25 (d, J=12.7 Hz, 4H), 1.15 (d, J=2.9 Hz, 2H), 0.92 (d, J=6.5 Hz, 9H). ESI-MS (EI⁺, m/2z): 528.8.

### Example 25: Synthesis of (2S,4R)-1-((S)-2-(3-(3-((4-((5-chloro-4-((2-(dimethylphosphoryl)phenyl)amino)pyrimidin-2-yl)amino)-5-methoxy-2-methylphenethyl)(methyl)amino)-3-oxopropoxy)propanamido)-3,3-dimethylbutanoyl)-4-hydroxy-N-((S)-1-(4-(4-methylthiazol-5-yl)phenyl)ethyl)pyrrolidine-2-carboxamide (71)

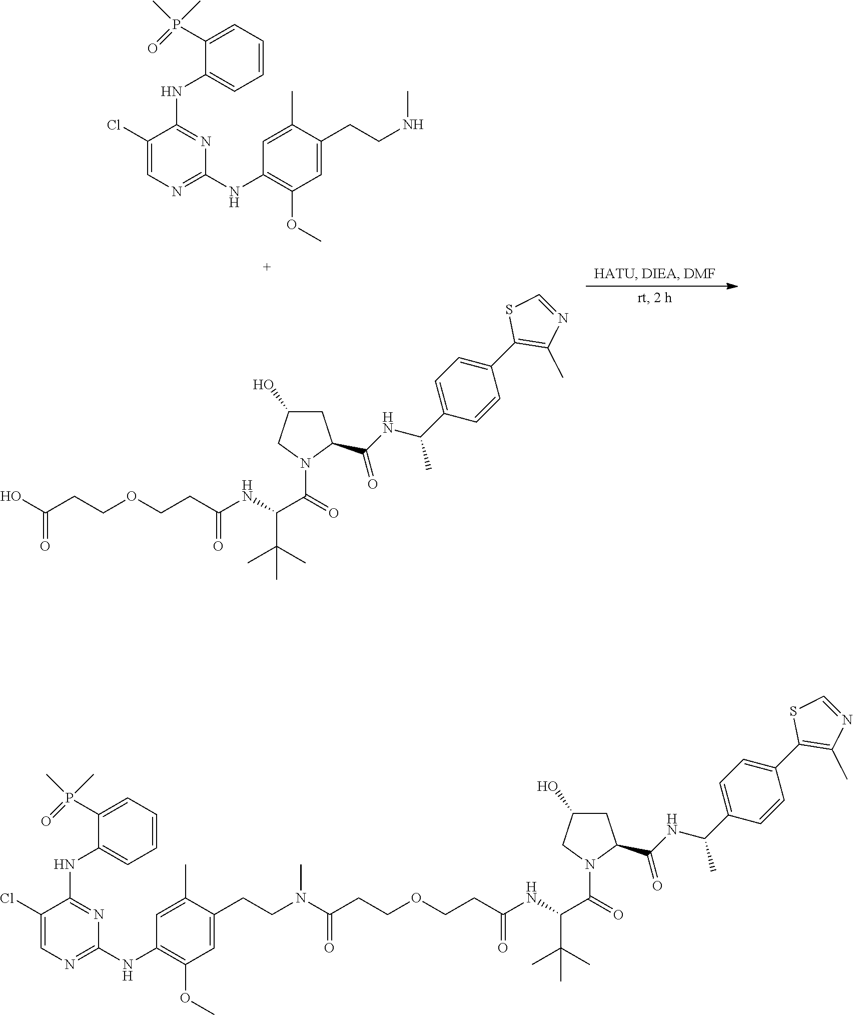 [SMILES: CNCCc1cc(OC)c(Nc2ncc(Cl)c(Nc3ccccc3P(C)(C)=O)n2)cc1C ; Cc1ncsc1-c1ccc([C@H](C)NC(=O)[C@@H]2C[C@@H](O)CN2C(=O)[C@@H](NC(=O)CCOCCC(=O)O)C(C)(C)C)cc1 ; COc1cc(CCN(C)C(=O)CCOCCC(=O)N[C@H](C(=O)N2C[C@H](O)C[C@H]2C(=O)N[C@@H](C)c2ccc(-c3scnc3C)cc2)C(C)(C)C)c(C)cc1Nc1ncc(Cl)c(Nc2ccccc2P(C)(C)=O)n1]

Compound 71 was synthesized based on similar procedure as compound 1. ¹H NMR (400 MHz, DMSO-d₆) δ 11.15 (s, 1H), 8.97 (s, 1H), 8.46 (s, 1H), 8.41-8.33 (m, 1H), 8.14 (s, 1H), 8.03 (s, 1H), 7.92-7.79 (m, 1H), 7.65-7.51 (m, 2H), 7.42-7.27 (m, 5H), 7.14 (s, 1H), 6.85 (d, J=4.6 Hz, 1H), 5.11 (s, 1H), 4.96-4.84 (m, 1H), 4.52 (t, J=8.6 Hz, 1H), 4.44 (d, J=17.2 Hz, 1H), 4.27 (s, 1H), 3.78 (s, 3H), 3.68-3.50 (m, 6H), 3.45 (d, J=10.1 Hz, 2H), 2.91 (d, J=45.6 Hz, 3H), 2.80-2.65 (m, 2H), 2.54 (d, J=5.5 Hz, 1H), 2.45 (s, 3H), 2.42-2.27 (m, 2H), 2.19 (d, J=5.1 Hz, 3H), 2.00 (d, J=7.4 Hz, 1H), 1.77 (d, J=13.5 Hz, 6H), 1.50-1.14 (m, 5H), 0.92 (d, J=10.1 Hz, 9H). ESI-MS (EI⁺, m/2z): 523.4.

### Example 26: Synthesis of N¹-(4-((5-chloro-4-((2-(dimethylphosphoryl)phenyl)amino)pyrimidin-2-yl)amino)-5-methoxy-2-methylphenethyl)-N⁹-((3S,5S)-1-((S)-3,3-dimethyl-2-((S)-2-(methylamino)propanamido)butanoyl)-5-(((R)-1-phenylpropyl)carbamoyl)pyrrolidin-3-yl)-N¹-methylnonanediamide (75)

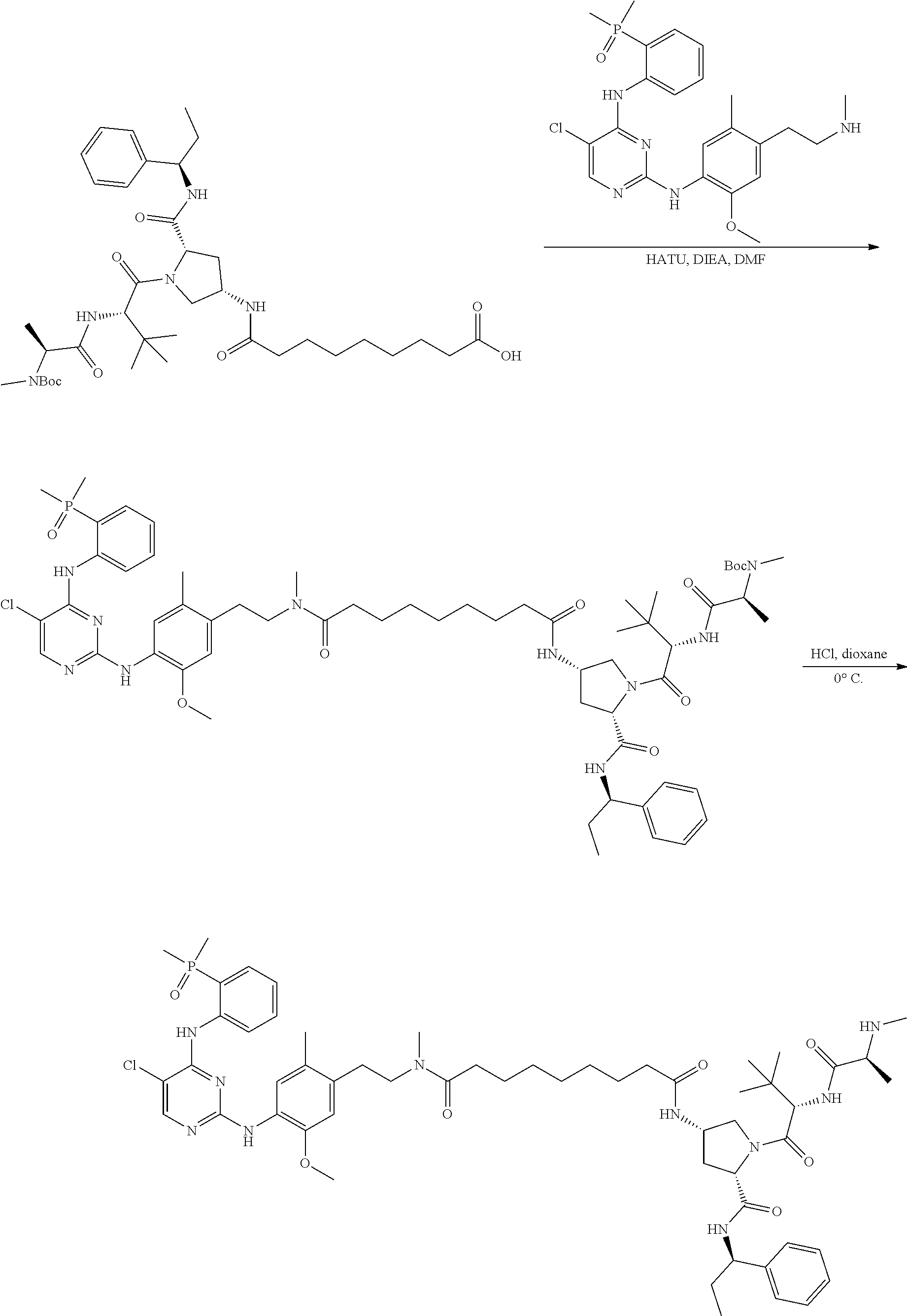 [SMILES: CC[C@@H](NC(=O)[C@@H]1C[C@H](NC(=O)CCCCCCCC(=O)O)CN1C(=O)[C@@H](NC(=O)[C@H](C)N(C)C(=O)OC(C)(C)C)C(C)(C)C)c1ccccc1 ; CNCCc1cc(OC)c(Nc2ncc(Cl)c(Nc3ccccc3P(C)(C)=O)n2)cc1C ; CC[C@@H](NC(=O)[C@@H]1C[C@H](NC(=O)CCCCCCCC(=O)N(C)CCc2cc(OC)c(Nc3ncc(Cl)c(Nc4ccccc4P(C)(C)=O)n3)cc2C)CN1C(=O)[C@@H](NC(=O)[C@H](C)N(C)C(=O)OC(C)(C)C)C(C)(C)C)c1ccccc1 ; CC[C@@H](NC(=O)[C@@H]1C[C@H](NC(=O)CCCCCCCC(=O)N(C)CCc2cc(OC)c(Nc3ncc(Cl)c(Nc4ccccc4P(C)(C)=O)n3)cc2C)CN1C(=O)[C@@H](NC(=O)[C@H](C)NC)C(C)(C)C)c1ccccc1]

tert-butyl ((2S,4S)-4-(9-((4-((5-chloro-4-((2-(dimethylphosphoryl)phenyl)amino)pyrimidin-2-yl)amino)-5-methoxy-2-methylphenethyl)(methyl)amino)-9-oxononanamido)-1-((S)-3,3-dimethyl-2-((S)-2-(methylamino)propanamido)butanoyl)pyrrolidine-2-carbonyl)((R)-1-phenylpropyl)carbamate was synthesized based on similar procedure as compound 1.

HCl in dioxane (1.0 mL) was added to a solution of compound 1031-1 (55 mg, 0.047 mmol) in DCM. The resulting mixture stirred at rt for 2 hours. After TLC showed reaction completion, the reaction mixture was concentrated in vacuo to afford the crude product. The crude product was purified by prep-HPLC to afford the title compound as a yellow solid (46.4 mg, 85.2%). ¹H NMR (400 MHz, DMSO-d₆) δ 11.88 (d, J=26.9 Hz, 1H), 9.35-9.26 (m, 1H), 8.85 (dt, J=11.9, 5.9 Hz, 1H), 8.59 (d, J=8.2 Hz, 1H), 8.45-8.27 (m, 3H), 8.15 (t, J=7.1 Hz, 1H), 7.65 (dd, J=13.7, 7.8 Hz, 1H), 7.49-7.32 (m, 4H), 7.30-7.15 (m, 4H), 6.90 (d, J=9.2 Hz, 1H), 4.71-4.65 (m, 1H), 4.43 (t, J=8.1 Hz, 2H), 4.25 (dd, J=13.2, 7.1 Hz, 1H), 4.06 (t, J=8.2 Hz, 1H), 3.95-3.90 (m, 1H), 3.78 (s, 3H), 3.67 (s, 1H), 3.45 (s, 1H), 3.27-3.23 (m, 1H), 2.96 (s, 2H), 2.86 (s, 1H), 2.81 (t, J=7.1 Hz, 1H), 2.76-2.71 (m, 1H), 2.47-2.36 (m, 4H), 2.26 (t, J=7.2 Hz, 1H), 2.18 (d, J=9.4 Hz, 3H), 2.00 (dd, J=15.6, 7.7 Hz, 3H), 1.79 (d, J=13.6 Hz, 8H), 1.67-1.57 (m, 1H), 1.52-1.40 (m, 3H), 1.32 (d, J=6.8 Hz, 4H), 1.23 (d, J=6.4 Hz, 5H), 1.11 (d, J=21.5 Hz, 3H), 0.99-0.80 (m, 12H). ESI-MS (EI, m/2z): 536.2.

### Example 27: Synthesis of 3-(5-(7-((4-((5-chloro-4-((2-(isopropylsulfonyl)phenyl)amino)pyrimidin-2-yl)amino)-5-methoxy-2-methylphenethyl)amino)hept-1-yn-1-yl)-1-oxoisoindolin-2-yl)piperidine-2,6-dione (90)

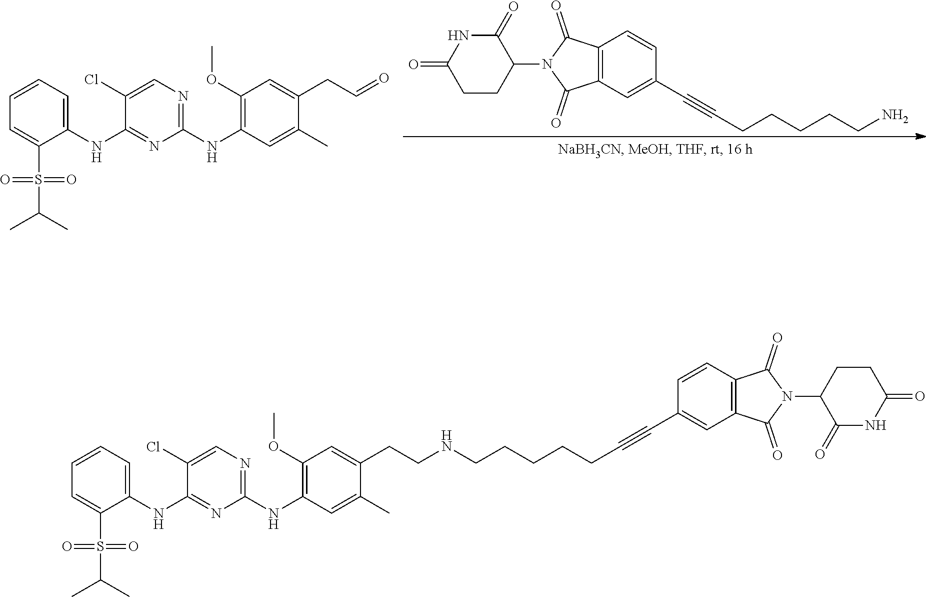

Following general procedure for reductive amination (Example 13) afforded the title compound as a white powder (19.7 mg, 7.56%). ¹H NMR (400 MHz, DMSO-d₆) δ 8.51 (d, J=6.9 Hz, 1H), 8.25 (d, J=8.6 Hz, 2H), 7.85 (dt, J=16.2, 7.6 Hz, 4H), 7.63 (t, J=7.2 Hz, 1H), 7.48 (s, 1H), 7.34 (t, J=6.5 Hz, 1H), 5.21-5.10 (m, 1H), 3.72 (s, 3H), 3.67-3.56 (m, 2H), 3.45 (dq, J=13.0, 6.2 Hz, 2H), 2.89 (ddd, J=17.3, 13.8, 5.1 Hz, 2H), 2.62 (s, 2H), 2.14 (d, J=8.9 Hz, 3H), 2.11-1.94 (m, 2H), 1.57 (d, J=42.8 Hz, 3H), 1.44 (d, J=3.6 Hz, 5H), 1.23 (s, 2H), 1.16 (d, J=6.7 Hz, 6H). ESI-MS (EI⁺, m/z): 841.1.

### Example 28: Synthesis of 3-(4-(7-((4-((5-chloro-4-((2-(isopropylsulfonyl)phenyl)amino)pyrimidin-2-yl)amino)-5-methoxy-2-methylphenethyl)amino)hept-1-yn-1-yl)-1-oxoisoindolin-2-yl)piperidine-2,6-dione (91)

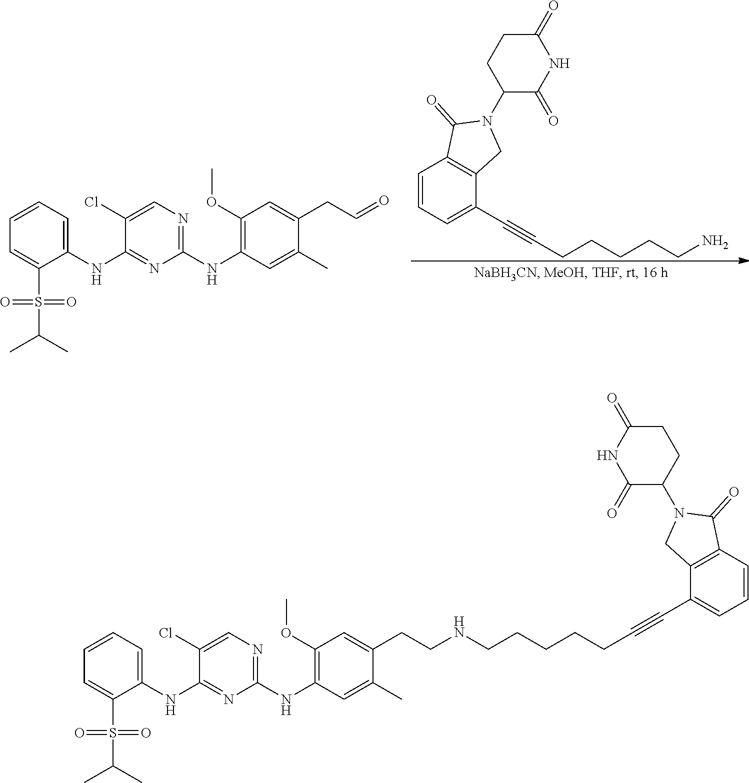

Following general procedure for reductive amination (Example 13) afforded the title compound as a yellow powder (130 mg, 36.3%). ¹H NMR (400 MHz, DMSO-d₆): δ 9.48 (br, 1H), 8.50 (d, J=8.0 MHz, 1H), 8.25 (s, 1H), 8.22 (s, 1H), 7.82 (dd, J=1.6, 7.6 MHz, 1H), 7.70 (d, J=7.2 MHz, 1H), 7.63-7.59 (m, 1H), 7.51 (t, J=7.2 MHz, 1H), 7.45 (s, 1H), 7.33 (t, J=7.6 MHz, 1H), 6.84 (s, 1H), 5.16-5.11 (m, 1H), 4.43 (dd, J=17.6, 56 MHz, 2H), 3.75 (s, 3H), 3.44-3.42 (m, 1H), 2.91-2.69 (m, 6H), 2.60-2.56 (m, 1H), 2.47-2.42 (m, 1H), 2.13 (s, 3H), 2.03-1.98 (m, 1H), 1.88 (s, 2H), 1.61-1.46 (m, 6H), 1.16 (s, 3H), 1.14 (s, 3H). ESI-MS (EI⁺, m/z): 826.50.

### Example 29: Synthesis of [3-(5-(7-((4-((5-chloro-4-((2-(isopropylsulfonyl)phenyl)amino)pyrimidin-2-yl)amino)-5-methoxy-2-methylphenethyl)amino)hept-1-yn-1-yl)-1-oxoisoindolin-2-yl)piperidine-2,6-dione (92)

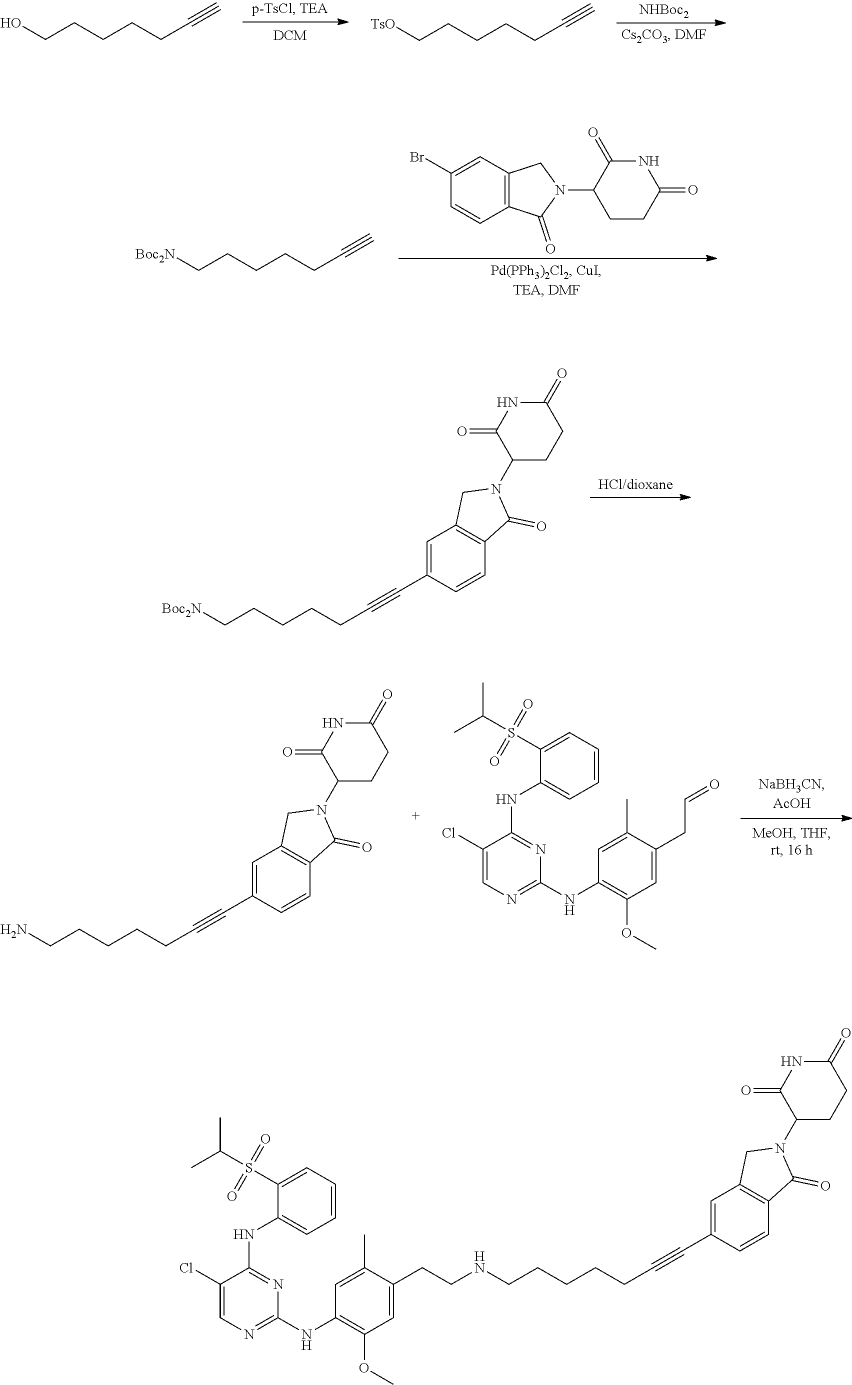

To a solution of hept-6-yn-1-ol (10 g, 89.21 mmol) in DCM (100 mL) was added triethylamine (13.5 g, 133.82 mmol) and the mixture was stirred at 0° C. After 30 minutes, p-TsCl (20 g, 107.06 mmol) was added and the solution was stirred for 4 hours. After TLC showed reaction completion, the solvent was removed under reduced pressure and the residue was purified by column chromatography eluted with (petroleum ether/ethyl acetate=20/1 to 15/1) to afford hept-6-yn-1-yl 4-methylbenzenesulfonate as a colorless, transparent liquid (12 g, 50.63%).

To a solution of di-tert-butyl iminodicarboxylate (2.4 g, 11.27 mmol) in 10 mL of DMF was added Cs₂CO₃ (3.7 g, 11.27 mmol) and the mixture stirred at 0° C. After 30 minutes, hept-6-yn-1-yl 4-methylbenzenesulfonate (2 g, 7.52 mmol) was added and the solution was stirred for 4 hours. After TLC showed reaction completion, the solvent was removed under reduced pressure and the residue was purified by column chromatography eluted with (petroleum ether/ethyl acetate=10/1) to afford tert-butyl hept-6-yn-1-ylcarbamate as a colorless, transparent liquid (1.9 g, 54.18%).

To a sealed tube, tert-butyl hept-6-yn-1-ylcarbamate (1 g, 3.11 mmol), 3-(6-bromo-3-oxo-1H-isoindol-2-yl)piperidine-2,6-dione (1.4 g, 4.66 mmol), CuI (84 mg, 0.44 mmol), and Pd(PPh)₃Cl₂ (153 mg, 0.22 mmol), were added and then the tube was degassed and charged with N₂, followed by the addition of anhydrous DMF (10 mL) and TEA (942 mg, 9.33 mmol). The reaction was stirred at 70° C. for 16 hours. After completion, the reaction mixture was diluted with H₂O (50 mL) and extracted with ethyl acetate. The solvent was removed under reduced pressure and the residue was purified by column chromatography eluted with (petroleum ether (PE)/ethyl acetate (EA)=10/1 to 3/1) to afford tert-butyl (7-(2-(2,6-dioxopiperidin-3-yl)-1-oxosoindolin-5-yl)hept-6-yn-1-yl)carbamate as a white solid (750 mg, 43.60%). ESI-MS (EI⁺, m/z): 552.35.

To a solution of tert-butyl (7-(2-(2,6-dioxopiperidin-3-yl)-1-oxosoindolin-5-yl)hept-6-yn-1-yl)carbamate (750 mg, 1.36 mmol) in THE (5 mL) was added HCl in dioxane (10 mL) and the mixture was stirred at rt for 1 hour. After TLC showed reaction completion, the solvent was removed under reduced pressure to afford 3-(5-(7-aminohept-1-yn-1-yl)-1-oxoisoindolin-2-yl)piperidine-2,6-dione as a white solid (437 mg, 91.04%). ESI-MS (EI⁺, m/z): 354.30.

Following the general procedure for reductive amination (Example 13) afforded the title compound as a white powder (25.6 mg, 5.48%). ¹H NMR (400 MHz, DMSO-d₆) δ 11.00 (s, 1H), 9.49 (s, 1H), 8.51 (d, J=8.1 Hz, 2H), 8.30-8.20 (m, 2H), 7.83 (dd, J=8.0, 1.5 Hz, 1H), 7.73-7.58 (m, 3H), 7.55-7.48 (m, 2H), 7.34 (td, J=7.7, 1.2 Hz, 2H), 6.85 (s, 2H), 5.10 (dd, J=13.3, 5.1 Hz, 1H), 4.37 (d, J=31.6 Hz, 2H), 3.77 (s, 3H), 3.50-3.39 (m, 1H), 3.06-2.81 (m, 8H), 2.72-2.54 (m, 2H), 2.31 (s, 2H), 2.14 (d, J=7.0 Hz, 3H), 2.05-1.95 (m, 1H), 1.70-1.42 (m, 6H), 1.16 (d, J=6.8 Hz, 6H). ESI-MS (EI⁺, m/z): 826.25.

### Example 30: Synthesis of 4-(7-(4-(4-((5-chloro-4-((2-(isopropylsulfonyl)phenyl)amino)pyrimidin-2-yl)amino)-5-methoxy-2-methylphenyl)piperidin-1-yl)hept-1-yn-1-yl)-2-(2,6-dioxopiperidin-3-yl)isoindoline-1,3-dione (93)

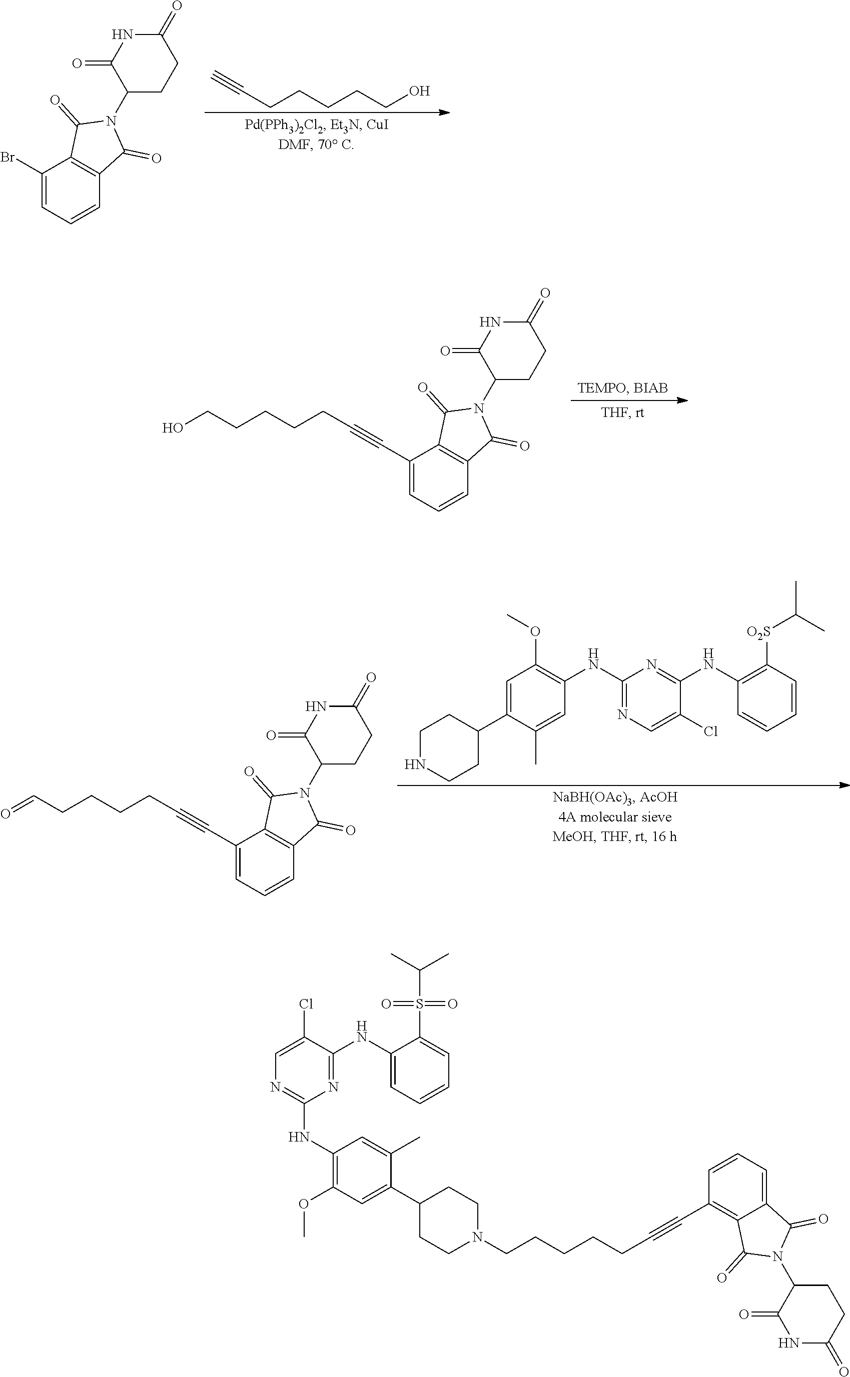

To a solution of 4-bromo-2-(2,6-dioxopiperidin-3-yl)isoindoline-1,3-dione (5.0 g, 14.0 mmol) in 50 mL of DMF was added hept-6-yn-1-ol (2.2 g, 19.6 mmol), Pd(PPh₃)₂Cl₂ (688.0 mg, 0.98 mmol), CuI (372.0 mg, 1.96 mmol) and Et₃N (4.2 g, 42.0 mmol). The resulting mixture was stirred at 70° C. under N₂ for 16 hours. After TLC showed reaction completion, the reaction solution was diluted with H₂O and extracted twice with EtOAc. The combined organic layers were washed with brine, dried over Na₂SO₄, filtered and concentrated in vacuo. The residue was purified by silica gel column chromatography (DCM:MeOH=80:1) to afford 2-(2,6-dioxopiperidin-3-yl)-4-(7-hydroxyhept-1-yn-1-yl)isoindoline-1,3-dione as a white solid (3.0 g, 57.9%). ESI-MS (EI⁺, m/z): 369.20.

To a solution of 2-(2,6-dioxopiperidin-3-yl)-4-(7-hydroxyhept-1-yn-1-yl)isoindoline-1,3-dione (1.1 g, 2.97 mmol) in THF (10 mL) was added BIAB (1.9 g, 5.94 mmol) and TEMPO (93.0 mg, 0.59 mmol). The resulting mixture was stirred at rt for 16 hours. After LCMS showed reaction completion, the reaction solution was diluted with aq. Na₂S₂O₃ and extracted twice with DCM, washed with aq. NaHCO₃, dried over Na₂SO₄, filtered and concentrated in vacuo. The residue was purified by silica gel column chromatography (DCM:MeOH=80:1) to afford 7-(2-(2,6-dioxopiperidin-3-yl)-1,3-dioxoisoindolin-4-yl)hept-6-ynal as a white solid (500.0 mg, 46.3%) ESI-MS (EI⁺, m/z): 367.00.

### Synthesis of 5-chloro-N⁴-(2-(isopropylsulfonyl)phenyl)-N²-(2-methoxy-5-methyl-4-(piperidin-4-yl)phenyl)pyrimidine-2,4-diamine

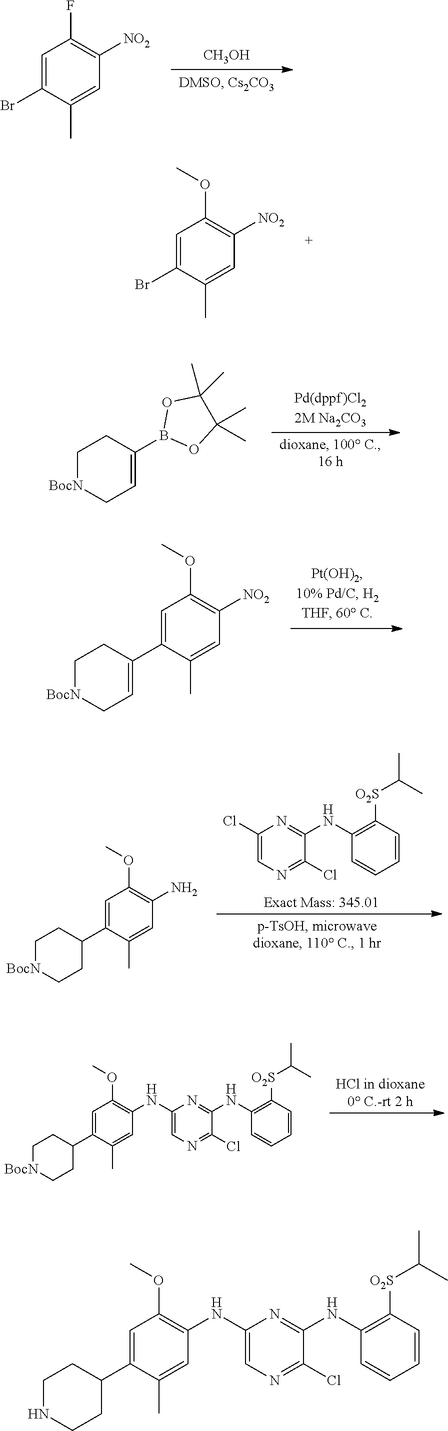

To a solution of 1-bromo-5-fluoro-2-methyl-4-nitrobenzene (10.0 g, 42.7 mmol) in DMSO (100 mL) was added Cs₂CO₃ (69.0 g, 214 mmol). CH₃OH (10 mL) was added dropwise at rt and the resulting mixture was stirred at 50° C. for 1 hour. After TLC showed reaction completion, the reaction solution was diluted with ice water and extracted twice with EtOAc, washed with brine, dried over Na₂SO₄, filtered and concentrated in vacuo. The residue was purified by silica gel column chromatography (PE:EA=3:1) to afford the compound 1-bromo-5-methoxy-2-methyl-4-nitrobenzene as a yellow solid (3.0 g, 28.8%). ¹H NMR (400 MHz, DMSO-d₆) δ 7.92 (d, J=0.8 Hz, 1H), 7.60 (s, 1H), 3.92 (s, 3H), 2.33 (d, J=0.6 Hz, 3H).

To a solution of 1-bromo-5-methoxy-2-methyl-4-nitrobenzene (500.0 mg, 2.1 mmol) in 10 mL of dioxane was added tert-butyl 4-(4,4,5,5-tetramethyl-1,3,2-dioxaborolan-2-yl)-3,6-dihydropyridine-1(2H)-carboxylate (826.0 mg, 2.7 mmol), Pd(dppf)₂Cl₂ (75.0 mg, 0.1 mmol) and Na₂CO₃ (434.0 mg, 4.1 mmol, 2.0 M in H₂O). The resulting mixture was stirred at 100° C. under N₂ for 16 hours. After TLC showed reaction completion, the reaction solution was diluted with aq. Na₂CO₃ and extracted twice with EtOAc, washed with brine, dried over Na₂SO₄, filtered and concentrated in vacuo. The residue was purified by silica gel column chromatography (PE:EA=3:1) to afford 2: tert-butyl 4-(5-methoxy-2-methyl-4-nitrophenyl)-3,6-dihydropyridine-1(2H)-carboxylate as a yellow solid (700.0 mg, 98.0%). ¹H NMR (400 MHz, DMSO-d₆) δ 7.73 (s, 1H), 7.05 (s, 1H), 5.69 (s, 1H), 3.98 (s, 2H), 3.89 (s, 4H), 3.55 (t, J=5.6 Hz, 3H), 2.36-2.29 (m, 6H), 2.21 (s, 4H), 1.44 (s, 9H).

To a solution of 2: tert-butyl 4-(5-methoxy-2-methyl-4-nitrophenyl)-3,6-dihydropyridine-1(2H)-carboxylate (200.0 mg, 0.6 mmol) in THF (5 mL) was added 10% Pd/C (200.0 mg) and Pt(OH)₂ (200.0 mg). The resulting mixture was stirred at 60° C. under H₂ for 16 hours. After TLC showed reaction completion, the reaction solution was filtered and concentrated in vacuo. The residue was purified by silica gel column chromatography (PE:EA=3:1) to afford tert-butyl 4-(4-amino-5-methoxy-2-methylphenyl)-3,6-dihydropyridine-1(2H)-carboxylate as a yellow solid (150.0 mg, 81.5%). ESI-MS (EI⁺, m/z): 265.10.

To a solution of tert-butyl 4-(4-amino-5-methoxy-2-methylphenyl)-3,6-dihydropyridine-1(2H)-carboxylate (100.0 mg, 0.32 mmol) in dioxane (1.5 mL) was added 2,5-dichloro-N-(2-(isopropylsulfonyl)phenyl)pyrimidin-4-amine (121.0 mg, 0.35 mmol) and TsOH (12.0 mg, 0.064 mmol). The resulting mixture was heated in a microwave at 110° C. for 1 hour. After LCMS showed reaction completion, the reaction solution was filtered and concentrated in vacuo. The residue was purified by silica gel column chromatography (PE:EA=3:1) to afford tert-butyl 4-(4-amino-5-methoxy-2-methylphenyl)piperidine-1-carboxylate a yellow solid (100.0 mg, 49.5%). ESI-MS (EI⁺, m/z): 530.10.

tert-Butyl 4-(4-amino-5-methoxy-2-methylphenyl)piperidine-1-carboxylate (1.2 g, 2.0 mmol) was added to HCl in dioxane (10.0 mL). The resulting mixture stirred at 0° C. for 2 hours. After TLC showed reaction completion, the reaction mixture was concentrated in vacuo to afford tert-butyl 4-(4-amino-5-methoxy-2-methylphenyl)piperidine-1-carboxylate as a yellow solid (1.0 g, 90.9%). ¹H NMR (400 MHz, DMSO-d₆) δ 9.94 (s, 1H), 9.18-9.03 (m, 1H), 8.94 (d, J=10.7 Hz, 1H), 8.39 (s, 1H), 8.23 (d, J=5.8 Hz, 1H), 7.89 (dd, J=8.0, 1.5 Hz, 1H), 7.68 (t, J=8.5 Hz, 1H), 7.47 (t, J=7.6 Hz, 1H), 7.32 (s, 1H), 6.80 (s, 1H), 3.77 (s, 4H), 3.56 (s, 2H), 3.47 (p, J=6.9 Hz, 1H), 3.35 (d, J=13.6 Hz, 3H), 3.08-2.95 (m, 2H), 2.10 (s, 3H), 1.94 (qd, J=13.2, 12.7, 2.8 Hz, 3H), 1.79 (d, J=15.8 Hz, 2H), 1.13 (d, J=6.8 Hz, 6H). ESI-MS (EI⁺, m/z): 530.30.

To a solution of 5-chloro-N⁴-(2-(isopropylsulfonyl)phenyl)-N²-(2-methoxy-5-methyl-4-(piperidin-4-yl)phenyl)pyrimidine-2,4-diamine (100.0 mg, 0.2 mmol) and 7-(2-(2,6-dioxopiperidin-3-yl)-1,3-dioxoisoindolin-4-yl)hept-6-ynal (73.0 mg, 0.2 mmol) in THF (4 mL) and MeOH (6 mL) was added 4 Å molecular sieve (100 mg) and HOAc (24.0 mg, 0.4 mmol). The resulting mixture was stirred at 0° C. for 30 minutes and then NaBH(OAc)₃ (127.0 mg, 0.6 mmol) was added. The resulting mixture was stirred at rt under N₂ for 16 hours. After LCMS showed reaction completion, the reaction mixture was purified with prep-HPLC to afford the title compound as a white solid (15.0 mg, 8.5%). ¹H NMR (400 MHz, DMSO-d₆) δ 11.13 (s, 1H), 9.49 (s, 1H), 8.51 (d, J=8.7 Hz, 1H), 8.26 (s, 1H), 8.22 (s, 1H), 7.88 (s, 1H), 7.85 (dd, J=5.8, 1.5 Hz, 2H), 7.81 (dd, J=8.0, 1.5 Hz, 1H), 7.59 (t, J=7.8 Hz, 1H), 7.41 (s, 1H), 7.36-7.29 (m, 1H), 6.84 (s, 1H), 5.15 (dd, J=12.8, 5.4 Hz, 1H), 3.75 (s, 3H), 3.44 (s, 3H), 3.02 (d, J=11.2 Hz, 3H), 2.88 (ddd, J=16.8, 13.7, 5.3 Hz, 2H), 2.69-2.58 (m, 1H), 2.53 (d, J=6.9 Hz, 3H), 2.36 (t, J=7.0 Hz, 3H), 2.14 (s, 3H), 2.09-1.99 (m, 4H), 1.78-1.65 (m, 1H), 1.65-1.58 (m, 4H), 1.50 (dt, J=13.4, 7.1 Hz, 4H), 1.23 (s, 1H), 1.16 (d, J=6.8 Hz, 6H). ESI-MS (EI⁺, m/z): 880.00.

### Example 31: Synthesis of 3-(4-(7-(4-(4-((5-chloro-4-((2-(isopropylsulfonyl)phenyl)amino)pyrimidin-2-yl)amino)-5-methoxy-2-methylphenyl)piperidin-1-yl)hept-1-yn-1-yl)-1-oxoisoindolin-2-yl)piperidine-2,6-dione (94)

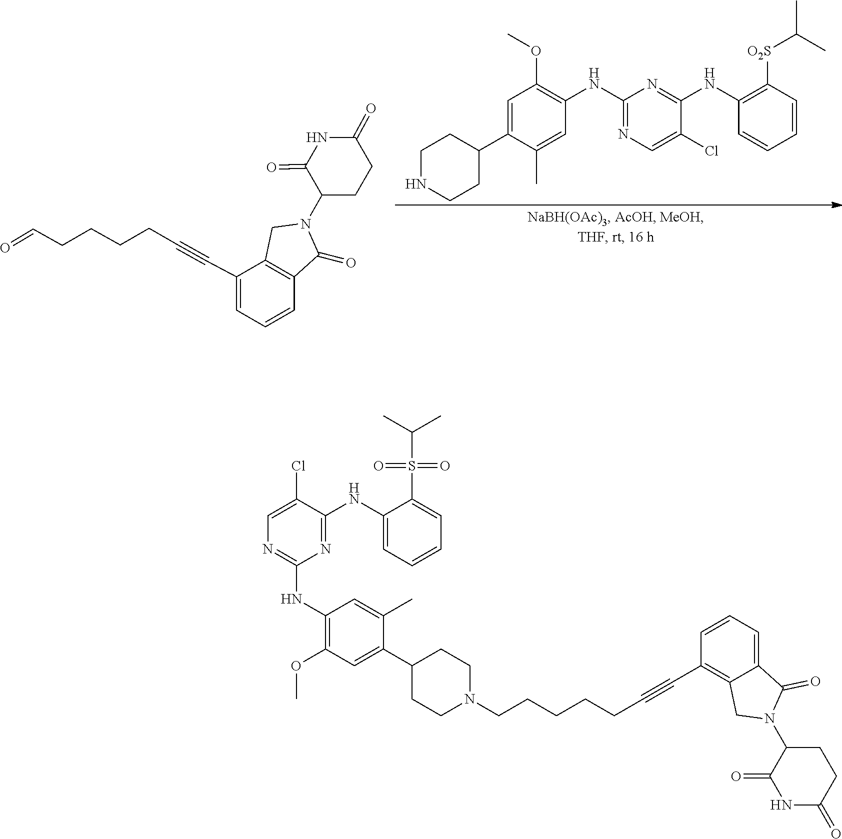 [SMILES: O=CCCCCC#Cc1cccc2c1CN(C1CCC(=O)NC1=O)C2=O ; COc1cc(C2CCNCC2)c(C)cc1Nc1ncc(Cl)c(Nc2ccccc2S(=O)(=O)C(C)C)n1 ; COc1cc(C2CCN(CCCCCC#Cc3cccc4c3CN(C3CCC(=O)NC3=O)C4=O)CC2)c(C)cc1Nc1ncc(Cl)c(Nc2ccccc2S(=O)(=O)C(C)C)n1]

To a solution of 5-chloro-N⁴-(2-(isopropylsulfonyl)phenyl)-N²-(2-methoxy-5-methyl-4-(piperidin-4-yl)phenyl)pyrimidine-2,4-diamine (50.0 mg, 0.1 mmol) and 7-(2-(2,6-dioxopiperidin-3-yl)-1-oxoisoindolin-4-yl)hept-6-ynal (35.0 mg, 0.1 mmol) in THF (2 mL) and MeOH (3 mL) was added 4 Å molecular sieve (50 mg) and HOAc (12.0 mg, 0.2 mmol). The resulting mixture was stirred at 0° C. for 30 minutes and then NaBH(OAc)₃ (63.0 mg, 0.3 mmol) was added. The resulting mixture was stirred at rt under N₂ for 16 hours. After LCMS showed reaction completion, the reaction mixture was purified by prep-HPLC to afford the title compound as a white solid (7.7 mg, 8.9%). ¹H NMR (400 MHz, DMSO-d₆) δ 11.01 (s, 1H), 9.51 (s, 1H), 8.49 (d, J=7.8 Hz, 1H), 8.34 (s, 1H), 8.23 (s, 1H), 7.83 (dd, J=8.0, 1.5 Hz, 1H), 7.72 (d, J=7.5 Hz, 1H), 7.67-7.63 (m, 1H), 7.59 (t, J=7.5 Hz, 1H), 7.53 (t, J=7.6 Hz, 1H), 7.46 (s, 1H), 7.36-7.30 (m, 1H), 6.77 (s, 1H), 5.16 (dd, J=13.2, 5.1 Hz, 1H), 4.50-4.28 (m, 2H), 3.76 (s, 3H), 3.59 (d, J=10.8 Hz, 2H), 3.36 (d, J=1.8 Hz, 1H), 3.11 (dd, J=6.1, 3.0 Hz, 2H), 3.08-3.02 (m, 1H), 3.02-2.95 (m, 1H), 2.94-2.86 (m, 1H), 2.60 (d, J=16.6 Hz, 1H), 2.53 (t, J=6.9 Hz, 2H), 2.45 (dd, J=13.2, 4.5 Hz, 1H), 2.18 (s, 3H), 2.07-1.98 (m, 2H), 1.93 (d, J=9.6 Hz, 4H), 1.75 (dd, J=9.8, 5.6 Hz, 2H), 1.68-1.61 (m, 3H), 1.46 (ddt, J=28.4, 15.2, 6.9 Hz, 4H), 1.16 (d, J=6.8 Hz, 6H). ESI-MS (EI⁺, m/z): 866.10.

### Example 32: Synthesis of 3-(4-(6-(4-(4-((5-chloro-4-((2-(isopropylsulfonyl)phenyl)amino)pyrimidin-2-yl)amino)-5-methoxy-2-methylphenyl)piperidin-1-yl)hex-1-yn-1-yl)-1-oxoisoindolin-2-yl)piperidine-2,6-dione (95)

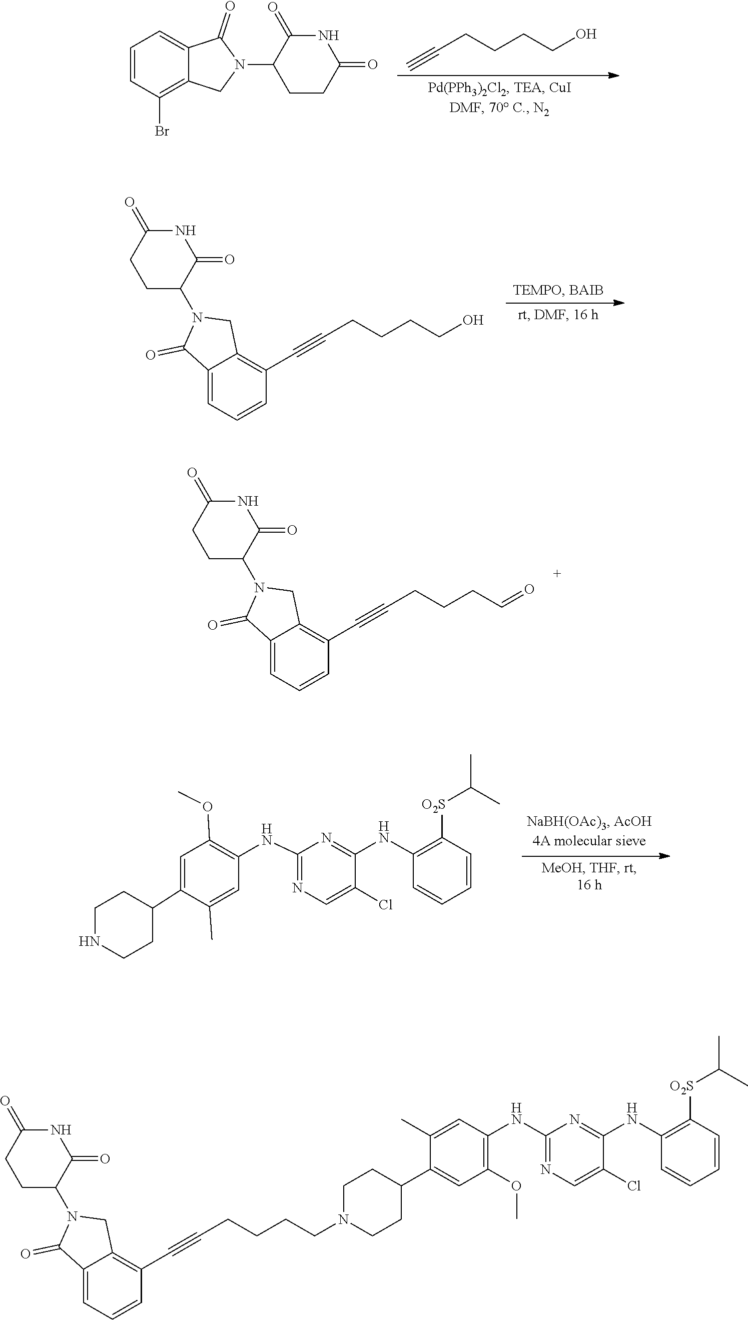

Hex-5-yn-1-ol (457.8 mg, 4.66 mmol), CuI (83.6 mg, 0.44 mmol), TEA (942 mg, 9.33 mmol), and Pd(PPh₃)₂Cl₂ (154.4 mg, 0.22 mmol) were added to a solution of 3-(7-bromo-3-oxo-1H-isoindol-2-yl)piperidine-2,6-dione (1.0 g, 3.11 mmol) in DMF (5 mL). The mixture was warmed to 70° C. and stirred overnight under nitrogen. The mixture was poured into H₂O (100 mL), extracted with CH₂Cl₂ (3×50 mL), and the combined organic phases were washed with brine and dried over Na₂SO₄. The residue was purified by silica gel column (CH₂Cl₂:MeOH=80:1) give compound 3-(4-(6-hydroxyhex-1-yn-1-yl)-1-oxoisoindolin-2-yl)piperidine-2,6-dione as a brown oil (300 mg, 28.3%). ESI-MS (EI⁺, m/z): 341.2.

BAIB (530 mg, 1.65 mmol) and TEMPO (153.7 mg, 0.98 mmol) were added to a solution of 3-(4-(6-hydroxyhex-1-yn-1-yl)-1-oxoisoindolin-2-yl)piperidine-2,6-dione (280 mg, 0.82 mmol) in DMF (2 mL). The mixture was stirred overnight at rt under nitrogen. The mixture was poured into H₂O (100 mL), quenched by EtOAc (3×50 mL), and the combined organic phases were washed with brine and dried over Na₂SO₄. The residue was purified by prep-HPLC to give compound 6-(2-(2,6-dioxopiperidin-3-yl)-1-oxoisoindolin-4-yl)hex-5-ynal as a yellow solid (80 mg, 28.86%). ¹H NMR (400 MHz, DMSO-d₆) δ 10.99 (s, 1H), 9.72 (t, J=1.3 Hz, 1H), 7.71 (dd, J=7.6, 1.0 Hz, 1H), 7.64 (dd, J=7.7, 1.1 Hz, 1H), 7.52 (t, J=7.6 Hz, 1H), 5.13 (dd, J=13.3, 5.1 Hz, 1H), 4.46 (d, J=17.8 Hz, 1H), 4.31 (d, J=17.7 Hz, 1H), 2.91 (ddd, J=18.0, 13.5, 5.3 Hz, 1H), 2.62 (td, J=7.2, 1.3 Hz, 2H), 2.59-2.51 (m, 2H), 2.49-2.40 (m, 2H), 2.06-1.95 (m, 1H), 1.83 (p, J=7.1 Hz, 2H). ESI-MS (EI⁺, m/z): 339.10.

A solution of 6-(2-(2,6-dioxopiperidin-3-yl)-1-oxoisoindolin-4-yl)hex-5-ynal (80 mg, 0.24 mmol) and 5-chloro-N⁴-(2-(isopropylsulfonyl)phenyl)-N²-(2-methoxy-5-methyl-4-(piperidin-4-yl)phenyl)pyrimidine-2,4-diamine (89 mg, 0.16 mmol) in 10.0 mL of MeOH and THF (v/v, 2:1) was treated with AcOH (18.8 mg, 0.31 mmol) and 4 A molecular sieve (2 g). The reaction mixture was stirred at rt under N₂ for 0.5 hours. NaBH(OAc)₃ (58.44 mg, 0.93 mmol) was added at 0° C. in portions, and the reaction mixture was stirred at rt for 16 hours. LCMS showed that most of SM was consumed. The mixture was partitioned between CH₂Cl₂ and water, and the combined organic phases were washed with water and brine, dried over Na₂SO₄ and concentrated in vacuo. The residue was purified by prep-TLC to give crude product. The crude product was purified by prep-HPLC to afford the title compound (12.1 mg, 9.57%). ¹H NMR (400 MHz, DMSO-d₆) δ 8.22 (s, 1H), 7.81 (d, J=9.4 Hz, 1H), 7.71 (d, J=7.5 Hz, 1H), 7.64 (d, J=7.0 Hz, 1H), 7.59 (t, J=7.7 Hz, 1H), 7.52 (t, J=7.6 Hz, 1H), 7.42 (s, 1H), 7.30 (s, 1H), 6.85 (s, 1H), 5.14 (dd, J=13.3, 5.1 Hz, 2H), 4.50-4.28 (m, 3H), 3.76 (s, 4H), 3.01 (d, J=10.0 Hz, 3H), 2.98-2.83 (m, 2H), 2.63 (d, J=25.8 Hz, 3H), 2.53 (d, J=6.9 Hz, 3H), 2.47-2.41 (m, 1H), 2.38 (s, 3H), 2.14 (s, 4H), 2.00 (d, J=12.8 Hz, 4H), 1.76-1.61 (m, 10H), 1.16 (d, J=6.8 Hz, 7H). ESI-MS (EI⁺, m/z): 852.50.

### Example 33: Synthesis of 3-(4-(5-(4-(4-((5-chloro-4-((2-(isopropylsulfonyl)phenyl)amino)pyrimidin-2-yl)amino)-5-methoxy-2-methylphenyl)piperidin-1-yl)pent-1-yn-1-yl)-1-oxoisoindolin-2-yl)piperidine-2,6-dione (96)

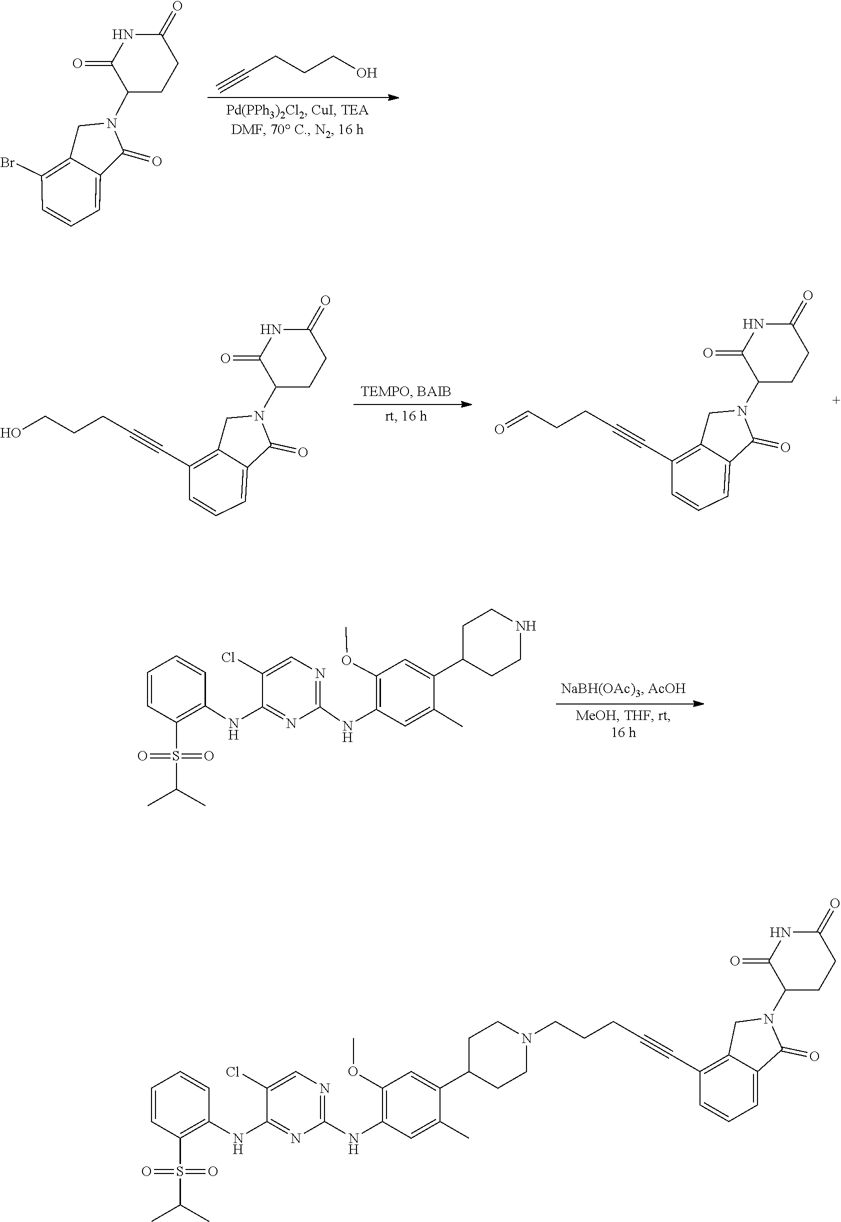

Compound 96 was synthesized based on a similar procedure as compound 95 and isolated as a white powder (5 mg, 6.33%). ¹H NMR (400 MHz, DMSO-d₆) δ 11.01 (s, 1H), 9.49 (s, 1H), 8.24 (d, J=15.1 Hz, 4H), 7.82 (dd, J=8.1, 1.3 Hz, 2H), 7.74-7.69 (m, 2H), 7.58 (br, 1H), 7.42 (s, 1H), 7.36-7.30 (m, 2H), 6.86 (s, 1H), 5.16 (dd, J=13.2, 5.1 Hz, 1H), 4.60-4.25 (m, 2H), 3.77 (s, 4H), 3.45 (dd, J=14.0, 7.2 Hz, 11H), 3.01 (d, J=10.5 Hz, 1H), 2.69-2.53 (m, 3H), 2.48-2.36 (m, 1H), 2.15 (s, 4H), 2.04 (d, J=10.2 Hz, 1H), 1.83-1.63 (m, 5H), 1.16 (d, J=6.8 Hz, 7H). ESI-MS (EI⁺, m/z): 838.45.

### Example 34: Synthesis of 3-(5-(3-(4-(4-((5-chloro-4-((2-(isopropylsulfonyl)phenyl)amino)pyrimidin-2-yl)amino)-5-methoxy-2-methylphenyl)piperidin-1-yl)prop-1-yn-1-yl)-1-oxoisoindolin-2-yl)piperidine-2,6-dione (97)

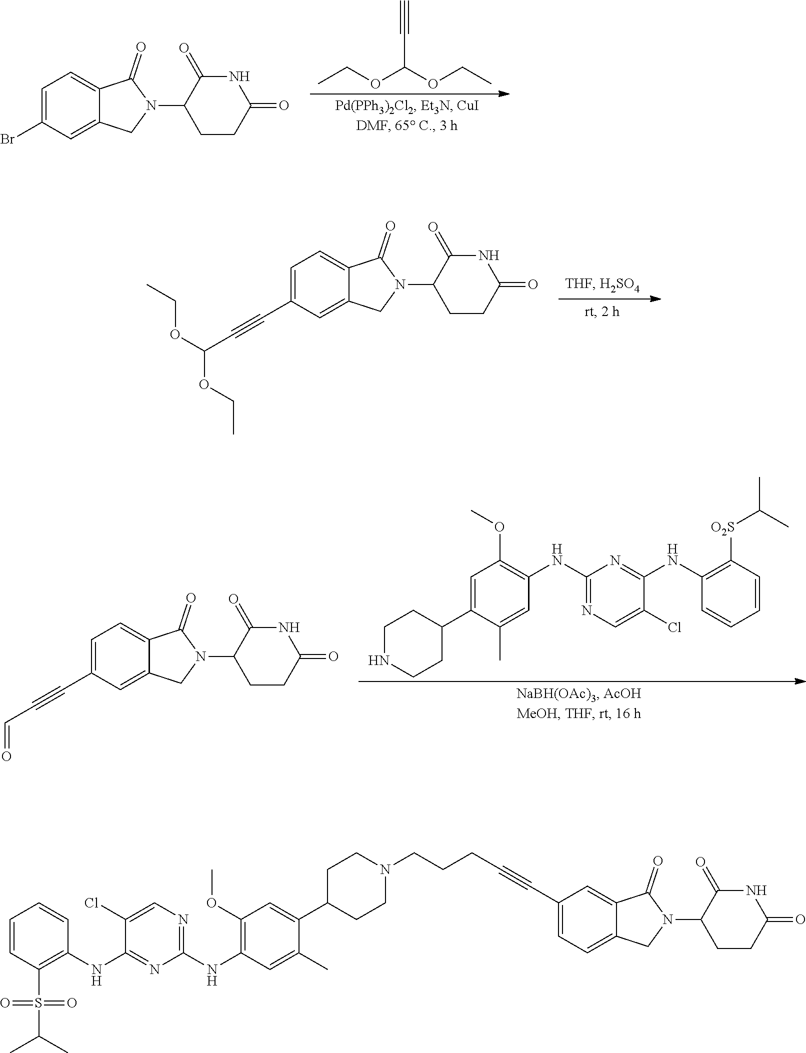

3,3-diethoxyprop-1-yne (620.0 mg, 4.8 mmol), Pd(PPh₃)₂Cl₂ (225.0 mg, 0.32 mmol), CuI (60.0 mg, 0.32 mmol) and Et₃N (969.0 mg, 9.6 mmol) were added to a solution of 3-(5-bromo-1-oxoisoindolin-2-yl)piperidine-2,6-dione (1.0 g, 3.2 mmol) in DMF (10 mL). The resulting mixture stirred at 70° C. under N₂ for 16 hours. After TLC showed reaction completion, the reaction solution was diluted with H₂O and extracted twice with EtOAc, washed with brine, dried over Na₂SO₄, filtered and concentrated in vacuo. The residue was purified by silica gel column chromatography (DCM:MeOH=20:1) to afford 3-(5-(3,3-diethoxyprop-1-yn-1-yl)-1-oxoisoindolin-2-yl)piperidine-2,6-dione as a white solid (500 mg, 41.6%). ESI-MS (EI⁺, m/z): 371.25.

H₂SO₄ (5 mL, 18 M) was added to a solution of 3-(5-(3,3-diethoxyprop-1-yn-1-yl)-1-oxoisoindolin-2-yl)piperidine-2,6-dione (300 mg, 1.0 mmol) in THF (5 mL) at 0° C. The resulting mixture stirred at rt for 2 hours. After TLC showed reaction completion, the reaction solution was diluted with H₂O and adjusted pH to ˜8 with aq. NaHCO₃, extracted twice with DCM, dried over Na₂SO₄, filtered and concentrated in vacuo to afford 3-[2-(2,6-dioxopiperidin-3-yl)-1-oxo-3H-isoindol-5-yl]prop-2-ynal as a white solid (200 mg, 71.4%). ESI-MS (EI⁺, m/z): 297.00.

4 Å molecular sieve (50 mg) and HOAc (12.0 mg, 0.2 mmol) were added to a solution of 5-chloro-N⁴-(2-(isopropylsulfonyl)phenyl)-N²-(2-methoxy-5-methyl-4-(piperidin-4-yl)phenyl)pyrimidine-2,4-diamine (50.0 mg, 0.1 mmol) and 3-[2-(2,6-dioxopiperidin-3-yl)-1-oxo-3H-isoindol-5-yl]prop-2-ynal (30.0 mg, 0.1 mmol) in THF (2 mL) and MeOH (3 mL). The resulting mixture stirred at 0° C. for 30 minutes and then NaBH(OAc)₃ (63.0 mg, 0.3 mmol) was added. The resulting mixture stirred at rt under N₂ for 16 hours. After LCMS showed reaction completion, the reaction mixture was purified with prep-HPLC to afford the title compound as a white solid (20 mg, 25.0%). ¹H NMR (400 MHz, DMSO-d₆) δ 11.00 (s, 1H), 9.49 (s, 1H), 8.51 (d, J=8.1 Hz, 1H), 8.28 (s, 1H), 8.22 (s, 1H), 7.81 (dd, J=8.0, 1.5 Hz, 1H), 7.73 (d, J=8.3 Hz, 2H), 7.58 (d, J=8.5 Hz, 2H), 7.41 (s, 1H), 7.36-7.29 (m, 5H), 6.89 (s, 1H), 5.12 (dd, J=13.3, 5.1 Hz, 1H), 4.51-4.29 (m, 2H), 3.77 (s, 3H), 3.61 (s, 2H), 3.45 (s, 2H), 3.03 (d, J=10.9 Hz, 3H), 2.96-2.86 (m, 2H), 2.69-2.57 (m, 4H), 2.46-2.39 (m, 1H), 2.36 (dt, J=9.4, 5.1 Hz, 4H), 2.16 (s, 3H), 2.02 (ddd, J=12.6, 5.4, 3.3 Hz, 2H), 1.84-1.69 (m, 6H), 1.16 (d, J=6.8 Hz, 6H). ESI-MS (EI⁺, m/z): 810.40.

### Example 35: Synthesis of (S)-7-(2-((4-((5-chloro-4-((2-(isopropylsulfonyl)phenyl)amino)pyrimidin-2-yl)amino)-5-methoxy-2-methylphenethyl)amino)ethoxy)-2-((S)-3,3-dimethyl-2-((S)-2-(methylamino)propanamido)butanoyl)-N-((R)-1,2,3,4-tetrahydronaphthalen-1-yl)-1,2,3,4-tetrahydroisoquinoline-3-carboxamide (100)

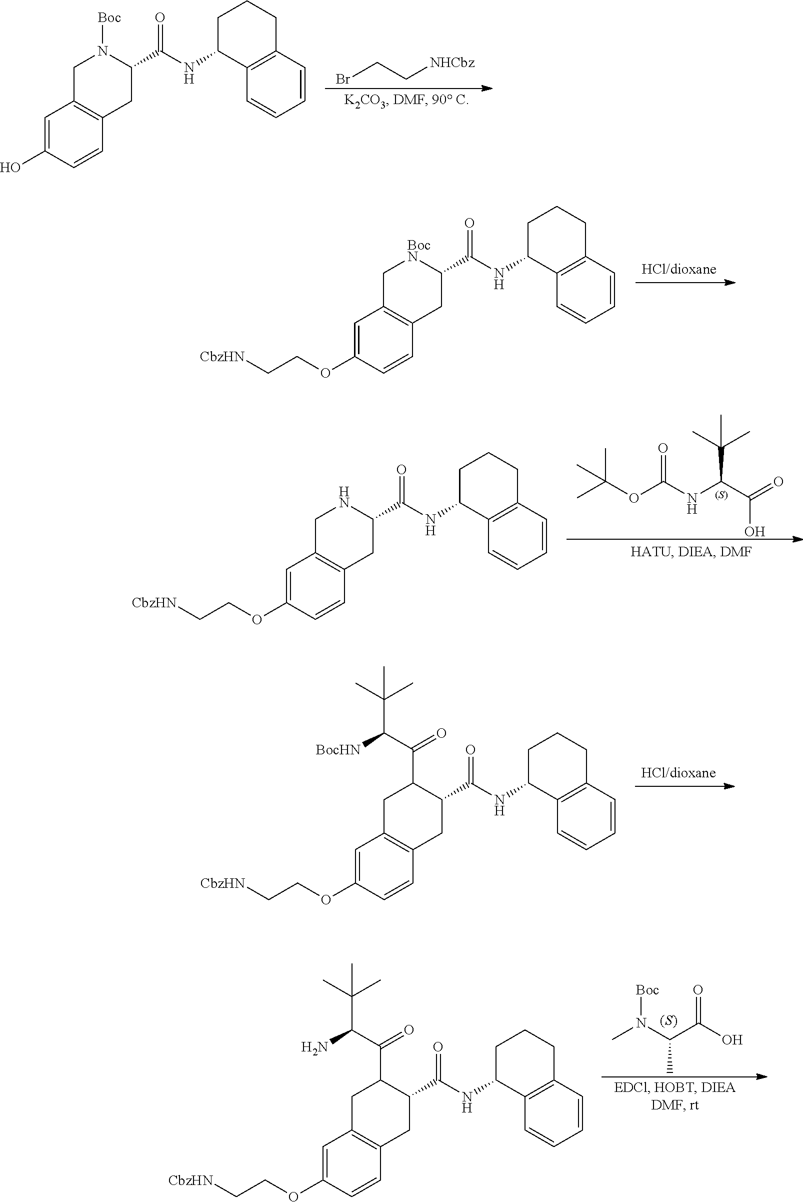

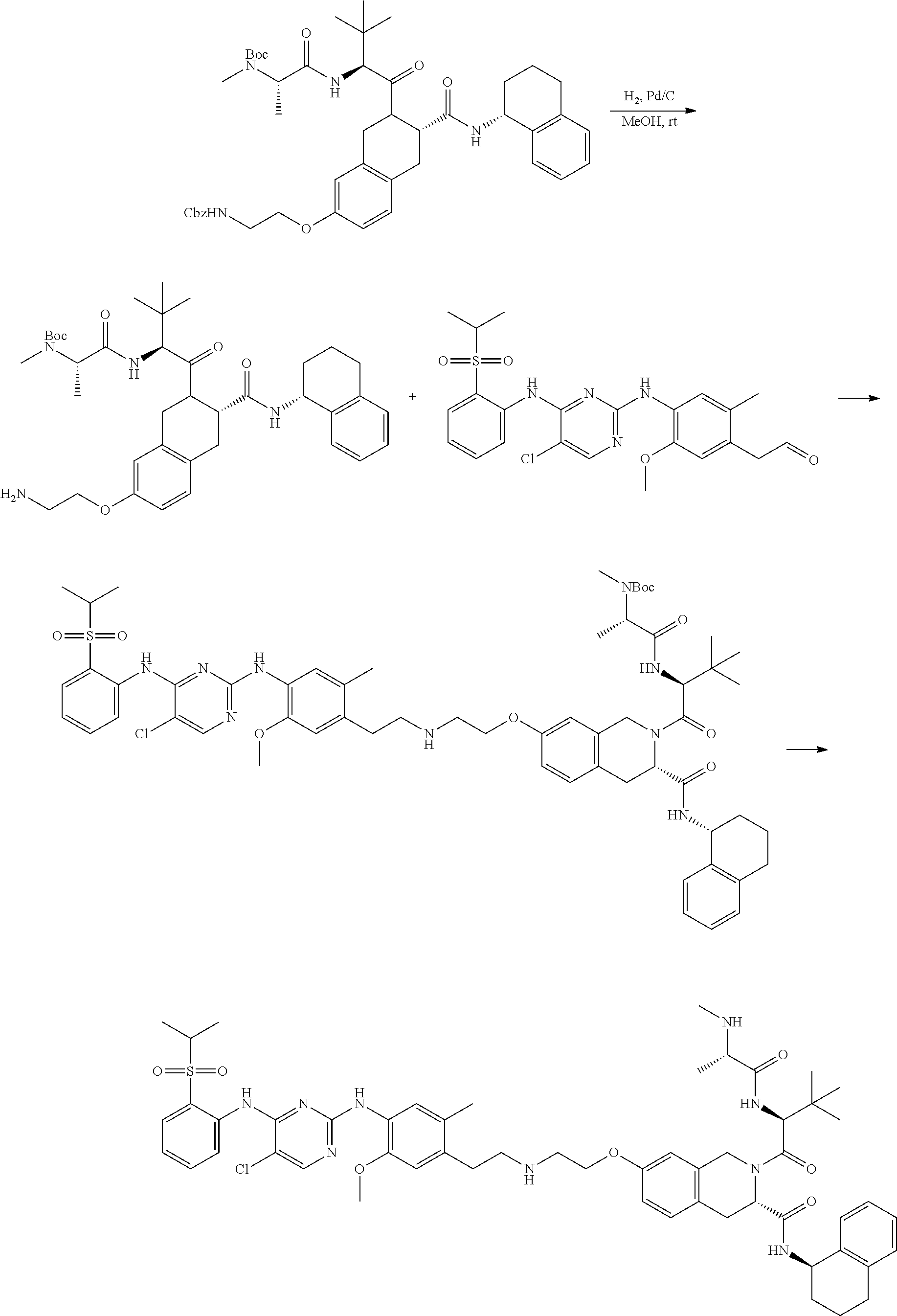

Following general procedure for reductive amination (Example 13) afforded title compound as a white powder (7 mg, 6.33%). ¹H NMR (400 MHz, DMSO-d₆) δ 9.48 (s, 1H), 8.51 (d, J=7.8 Hz, 1H), 8.27 (s, 1H), 8.22 (d, J=2.8 Hz, 2H), 8.20-8.16 (m, 1H), 7.94 (dd, J=22.1, 8.8 Hz, 1H), 7.84-7.78 (m, 1H), 7.61 (t, J=7.8 Hz, 1H), 7.44 (s, 1H), 7.35-7.28 (m, 1H), 7.16-6.96 (m, 5H), 6.92 (d, J=2.5 Hz, 1H), 6.87 (s, 1H), 6.81 (dd, J=9.0, 6.8 Hz, 1H), 4.99-4.82 (m, 3H), 4.80-4.61 (m, 3H), 4.05 (t, J=5.7 Hz, 2H), 3.74 (d, J=1.5 Hz, 3H), 3.46 (s, 1H), 3.45 (s, 2H), 3.43 (s, 2H), 3.05-2.96 (m, 4H), 2.89-2.82 (m, 2H), 2.80-2.64 (m, 4H), 2.19 (s, 1H), 2.14 (d, J=2.3 Hz, 5H), 1.90-1.51 (m, 4H), 1.15 (d, J=6.8 Hz, 6H), 1.08 (dd, J=14.9, 6.9 Hz, 4H), 1.04 (s, 6H), 0.94 (s, 2H). ESI-MS (EI⁺, m/z): 519.30.

### Example 36: Synthesis of 2-((5-chloro-2-((4-(2-((7-(2-(2,6-dioxopiperidin-3-yl)-1,3-dioxoisoindolin-4-yl)hept-6-yn-1-yl)amino)ethyl)-2-methoxy-5-methylphenyl)amino)pyrimidin-4-yl)amino)-N-methylbenzamide (101)

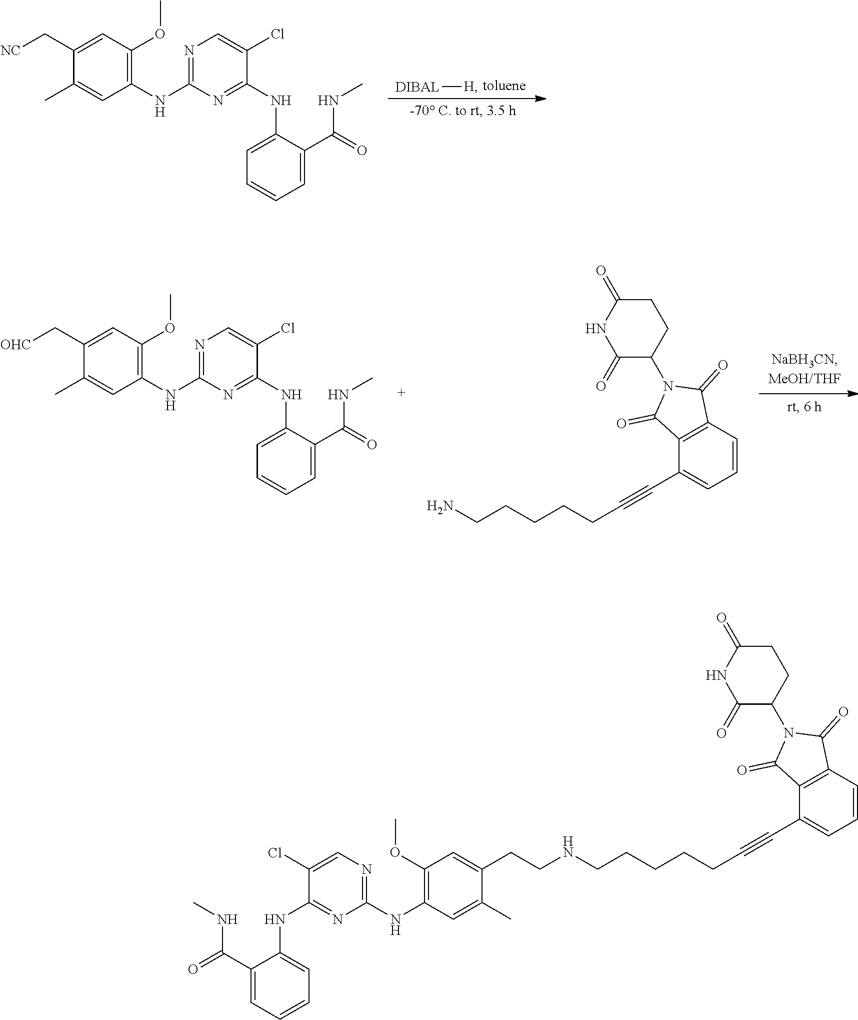

DIBAL-H (11.5 mL, 11.4 mmol) was added slowly to a solution of the compound 2-((5-chloro-2-((4-(cyanomethyl)-2-methoxy-5-methylphenyl)amino)pyrimidin-4-yl)amino)-N-methylbenzamide (500 mg, 1.14 mmol) in toluene (30 mL) at −70° C. under nitrogen atmosphere. The resulting mixture stirred at the same temperature for 0.5 hours, then warmed to room temperature, and continued to stir for 3 hours. 5% citric acid aqueous solution (100 mL) was added, stirred at room temperature for 1 hour, and then extracted with EtOAc (3×100 mL). The combined organic phases were washed with brine, dried over Na₂SO₄ and concentrated in vacuo. The crude product was purified by prep-TLC to afford 2-((5-chloro-2-((2-methoxy-5-methyl-4-(2-oxoethyl)phenyl)amino)pyrimidin-4-yl)amino)-N-methylbenzamide as a yellow solid (30 mg, 5.9%). ESI-MS (EI⁺, m/z): 340.14.

NaBH₃CN (11 mg, 0.17 mmol) was added to a solution of compound 2-((5-chloro-2-((2-methoxy-5-methyl-4-(2-oxoethyl)phenyl)amino)pyrimidin-4-yl)amino)-N-methylbenzamide (25 mg, 0.05 mmol) and 4-(7-aminohept-1-yn-1-yl)-2-(2,6-dioxopiperidin-3-yl)isoindoline-1,3-dione (25 mg, 0.06 mmol) in a solution of THF (2 mL) and MeOH (2 mL) at 0° C., and the mixture stirred at room temperature for 6 hours. The mixture was filtered and the filtrate was concentrated to 1.5 mL, and then purified by prep-HPLC to afford the title compound as a yellow solid (6 mg, 13.3%). ¹H NMR (400 MHz, DMSO-d₆) δ 11.61 (s, 1H), 8.74 (d, J=5.2 Hz, 1H), 8.58 (d, J=8.8 Hz, 1H), 8.35 (s, 1H), 8.16 (s, 1H), 8.10 (s, 1H), 7.88 (d, J=8.0 Hz, 1H), 7.86-7.82 (m, 2H), 7.73 (d, J=8.9 Hz, 1H), 7.55 (s, 1H), 7.39-7.33 (m, 1H), 7.09 (t, J=7.2 Hz, 1H), 6.86 (s, 1H), 5.15 (dd, J=12.8, 5.6 Hz, 1H), 3.77 (s, 3H), 2.94-2.81 (m, 4H), 2.80 (d, J=4.5 Hz, 3H), 2.79-2.66 (m, 5H), 2.60 (d, J=19.7 Hz, 1H), 2.53 (d, J=4.1 Hz, 1H), 2.47 (d, J=5.1 Hz, 1H), 2.18 (s, 3H), 2.09-2.00 (m, 1H), 1.62-1.44 (m, 7H). ESI-MS (EI⁺, m/z): 791.30.

### Example 37: Degradation Data for Ceritinib Analogs

NSCLC cell line H3122 was subjected to CRISPR to express EML4-ALK with HiBit-tag on its c-terminal. Single-cell clone was selected and expanded for the following assays that monitor endogenous EML4-ALK level in high throughput manner. Cells were seeded in 384-well white Tissue-culture plates at 3000 cells/well for 8 hours. Subsequently, cells were titrated with degraders for 16 hours at 0.00867-20 μM in triplicate by the Hewlett-Packard® D300 digital dispenser. HiBit signal was assessed using Nano-Glo® HiBit Lytic Detection System (Promega™). Degrader-treated was normalized by DMSO-treated controls and DC₅₀ and Dₘₐₓ were generated by GraphPad Prism® 8.0. The results in Table 1 indicate that the bispecific compounds potently degrade ALK.

TABLE 1

| | Degradation | | | | | | CTG | | | |
|---|---|---|---|---|---|---|---|---|---|---|
| Compound | DC50 (μM) | Dmax (μM) | DC50 (μM) | Dmax (μM) | Avg. DC50 (μM) | Avg. Dmax (μM) | IC50 (nM) | | LogP | LogD |
| TL-1 [symbol] -112 | 0.058 | 70.4 | 0.048 | 67.7 | 0.053 | 59.1 | 51.1 | | 5.11 | 3.78 |
| 1 | 0.032 | 25.8 | — | — | 0.032 | 23.8 | 44.8 | | 4.9 | 4.9 |
| 2 | 0.067 | 24.1 | 0.046 | [symbol] .7 | 0.057 | 31.4 | 76.9 | | 5.79 | 5.79 |
| 50 | 0.057 | 51.5 | 0.024 | 24.9 | 0.041 | 38.2 | 20.6 | | 6.08 | 3.98 |
| 3 | — | — | 0.346 | 38.4 | — | — | 136.1 | | 7.13 | 7.13 |
| 4 | 0.100 | 44.8 | 0.034 | 33.0 | 0.067 | 38.9 | 25.1 | | 4.52 | 4.52 |
| 5 | 0.239 | 44.5 | — | — | 0.23 [symbol] | 44.5 | 149 | | 3.52 | 3.52 |
| 6 | 0.124 | 34.0 | 0.266 | 30.4 | 0.195 | 32.2 | 11.6 | | 4.41 | 4.41 |
| 7 | 0.091 | 50.8 | 0.036 | 44.8 | 0.063 | 47.8 | 15.7 | | 5.7 | 5.74 |
| 8 | 0.171 | 44.3 | — | — | 0.171 | 44.8 | 75 | | 3.1 | 3.13 |
| 9 | 0.265 | 54.1 | — | — | 0.265 | 54.1 | 121.6 | | 5.78 | 5.78 |
| 10 | 0.203 | 43.4 | — | — | 0.203 | 43.4 | 125.4 | | 6.67 | 6.67 |
| 49 | 0.151 | 112.2 | 0.105 | 71.1 | 0.128 | 91.6 | 66.9 | 96.32 | 6.5 | 4.03 |
| 51 | — | — | — | — | — | — | — | | — | — |
| 11 | 0.502 | 53.3 | — | — | 0.302 | 53.3 | 219.4 | | 4.75 | 4.75 |
| 39 | 0.402 | 79.1 | 0.184 | 99.3 | 0.293 | 89.2 | 1475 | 414.5 | 9.22 | 5.44 |
| 40 | 0.392 | 68.6 | 0.145 | 41.6 | 0.269 | 55.1 | 149.3 | | 7.45 | 7.45 |
| 41 | 0.626 | 56.2 | — | — | 0.626 | 56.2 | 176 | | 7.89 | 7.89 |
| 42 | 0.777 | 47.3 | — | — | 0.777 | 47.3 | 1309 | | 8.34 | 8.34 |
| 43 | 1.227 | 38.1 | — | — | 1.227 | [symbol] .1 | 288.7 | | 6.07 | 3.6 |
| 44 | 1.251 | 53. [symbol] | — | — | 1.251 | 53. [symbol] | 252.5 | | 6.93 | 4.37 |
| 45 | 0.390 | 51.0 | — | — | 0.390 | 51.0 | 106.2 | | 7.82 | 5.26 |
| 46 | 1.395 | 58.0 | 0.206 | 34.5 | 0.801 | 51.3 | 315.6 | | 5.86 | 3.78 |
| 47 | 1.099 | 81.9 | 0.347 | 95.6 | 0.723 | 88.7 | 285.1 | | 5.81 | 3.73 |
| 48 | 0.961 | 77.2 | 0.611 | 108.4 | 0.786 | 92.8 | 388.4 | | 6.37 | 6.37 |
| 52 | 0.214 | 54.8 | 0.107 | 49.4 | 0.160 | 52.1 | 67.7 | | 5.62 | 5.62 |
| 53 | 0.113 | 64.9 | 0.072 | 68.2 | 0.093 | 66.5 | 121.2 | | — | — |
| 54 | 0.067 | 68.4 | 0.057 | 68.2 | 0.062 | [symbol] .3 | 73.6 | | — | — |
| 55 | 0.624 | 74.6 | 0.360 | 101.8 | 0.492 | 88.2 | 295.6 | | 7.33 | 6.1 |
| 56 | 0.163 | 47.1 | 0.226 | 89.4 | 0.195 | 68.2 | 359.9 | | — | — |

[symbol]

indicates data missing or illegible when filed

### Example 38: Degradation Data for Ceritinib Analogs

NSCLC cell line H3122 was subjected to CRISPR to express EML4-ALK with HiBit-tag on its c-terminus. A single-cell clone was selected and expanded for the following assays that monitor endogenous EML4-ALK level in high throughput manner. Cells were seeded in 384-well white Tissue-culture plates at 3000 cells/well for 8 hours. Subsequently, cells were titrated with degraders for 16 hours at 0.00867-20 μM in triplicate by the Hewlett-Packard® D300 digital dispenser. HiBit signal was assessed using Nano-Glo® HiBit Lytic Detection System (Promega™). Degrader-treated cells were normalized by DMSO-treated controls and DC₅₀ and Dₘₐₓ were generated by GraphPad Prism® 8.0. The results in Table 2 show that the bispecific compounds potently degrade ALK.

TABLE 2
Degradation data for brigatinib analogs.

| | Degradation | | | | | | CTG | | |
|---|---|---|---|---|---|---|---|---|---|
| Compound | DC50 (μM) | Dmax (μM) | DC50 (μM) | Dmax (μM) | Avg. DC50 (μM) | Avg. Dmax (μM) | IC50 (nM) | LogP | LogD |
| Brigatinib | — | — | — | — | — | — | 21 | | |
| 57 | 9.341 | 13.3 | — | — | 9.34 | 13.3 | >10000 | 1.98 | 1.98 |
| 58 | — | — | — | — | — | — | >10000 | 2.77 | 2.77 |
| 59 | 3.740 | 33.7 | — | — | 3.74 | 33.7 | 1722 | 3.96 | 3.96 |
| 60 | — | — | — | — | — | — | >10000 | 1.4 | 1.4 |
| 61 | 9.038 | 29.5 | 8.37 | 39.6 | 8.70 | 34.5 | >10000 | 4.06 | 4.06 |
| 62 | 4.382 | 27.8 | — | — | 4.38 | 27.8 | >10000 | 4.85 | 4.85 |
| 19 | 0.160 | 31.3 | — | — | — | — | — | | |
| 63 | 3.907 | 27.3 | — | — | 3.91 | 27.3 | >10000 | 2.77 | 2.77 |
| 64 | — | — | — | — | — | — | 301.3 | 3.53 | 3.53 |
| 65 | 2.560 | 26.2 | — | — | 2.56 | 26.2 | 1103 | 4.32 | 4.32 |
| 66 | 1.727 | 23.6 | 0.67 | 49.3 | 1.20 | 36.5 | 1633 | 5.51 | 5.51 |
| 67 | — | — | — | — | — | — | 2290 | 2.95 | 2.95 |
| 68 | — | — | — | — | — | — | — | 6.62 | 6.62 |
| 69 | — | — | — | — | — | — | 476.6 | 6.23 | 6.23 |
| 70 | — | — | — | — | — | — | 370.9 | 5.83 | 5.83 |
| 71 | 0.543 | 50.9 | 0.15 | 43.7 | 0.35 | 47.3 | 222.1 | 4.08 | 4.08 |
| 72 | 8.815 | 37.9 | 3.59 | 37.9 | 6.20 | 37.9 | 2950 | 4.76 | 2.06 |
| 73 | 3.274 | 59.6 | 1.18 | 62.0 | 2.23 | 60.8 | 787.6 | 4.61 | 1.75 |
| 74 | 0.506 | 47.1 | 0.48 | 47.2 | 0.49 | 47.1 | — | | |
| 75 | 0.381 | 42.1 | — | — | — | — | — | | |

### Example 39: Degradation Data

NSCLC cell line H3122 was subjected to CRISPR to express EML4-ALK with HiBit-tag on its C-terminus. A single-cell clone was selected and expanded for the following assays that monitor endogenous EML4-ALK level in high throughput manner. Cells were seeded in 384-well white Tissue-culture plates at 3000 cells/well for 8 hours. Subsequently, cells were titrated with degraders for 16 hours at 0.00867-20 μM in triplicate by the Hewlett-Packard® D300 digital dispenser. HiBit signal was assessed using Nano-Glo® HiBit Lytic Detection System (Promega™). Degrader-treated cells were normalized by DMSO-treated controls and DC₅₀ and Dₘₐₓ were generated by GraphPad Prism® 8.0. The results in Table 3 show that the inventive bispecific compounds tested potently degrade ALK and FAK.

TABLE 3
ALK and FAK Degradation Data

| | ALK Degradation | | FAK Degradation | | |
|---|---|---|---|---|---|
| | (H3122 cells) | | (H3122 cells) | | CTG |
| | DC₅₀ | Dmax | DC₅₀ | Dmax | IC₅₀ |
| Compound | (nM) | (%) | (nM) | (%) | (nM) |
| 90 | 46 | 74 | 16 | 92 | 93 |
| 91 | 15 | 69 | 4 | 85 | 50 |
| 92 | 17 | 93 | 4 | 93 | 50 |
| 93 | 49 | 82 | 24 | 89 | 202 |
| 94 | 2 | 69 | 2 | 86 | 18 |
| 95 | 1 | 73 | 1 | 89 | 4 |
| 96 | 3 | 79 | 2 | 92 | 12 |
| 97 | 7 | 65 | 8 | 80 | 20 |
| MS4740 | 10 | 77 | 31 | 97 | 73 |
| SIAIS117 | 9 | 43 | 10 | 70 | 42 |

MS4740-Zhang et al., Eur. J. Med. Chem. 151: 304-314 (2018)
SIAIS117-Sun et al., Eur. J. Med. Chem. 193: 112190 (2020)

### Example 40: Western Blots

The cells were collected and washed with PBS buffer. Cell lysates were prepared by using NP40 lysis buffer (Invitrogen™) supplemented with complete protease inhibitor cocktail (Roche), PhosSTOP™ phosphatase inhibitor cocktail (Roche) and PMSF (1 mM). The lysates were cleared by centrifugation and resolved using Bolt™ 4-12% Bis-Tris plus gels and Western blotted to detect proteins of interest. Antibodies to phospho-ALK^{Tyr1507} and tubulin (#14678S and #3873, Cell Signaling Technologies®) were used according to the manufacturers' instructions. For Western Blot visualization, Odyssey® Clx (Li-cor) was utilized.

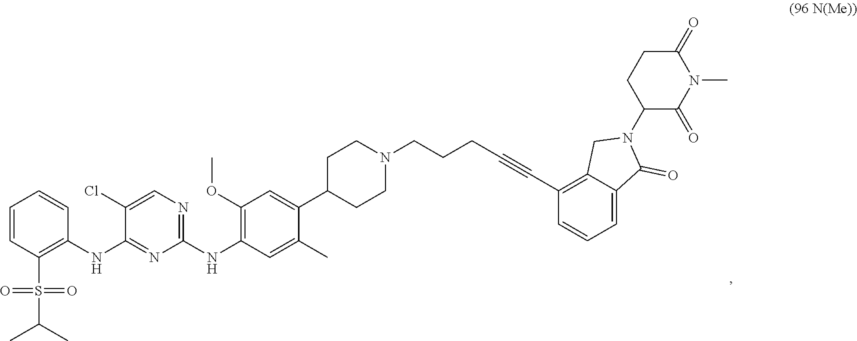 [SMILES: COc1cc(C2CCN(CCCC#Cc3cccc4c3CN(C3CCC(=O)N(C)C3=O)C4=O)CC2)c(C)cc1Nc1ncc(Cl)c(Nc2ccccc2S(=O)(=O)C(C)C)n1]

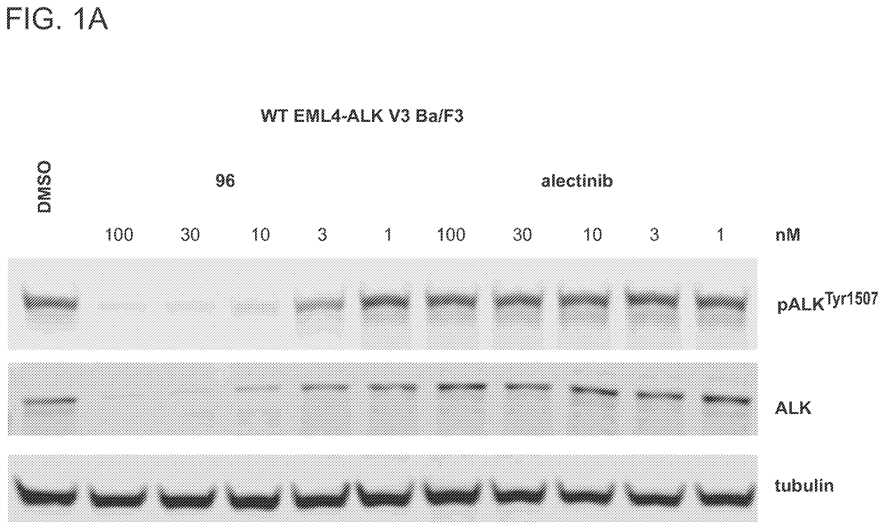

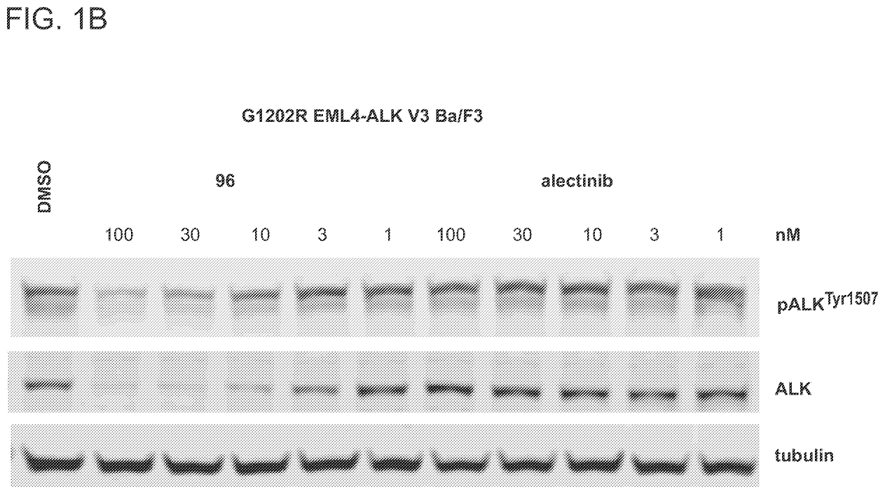

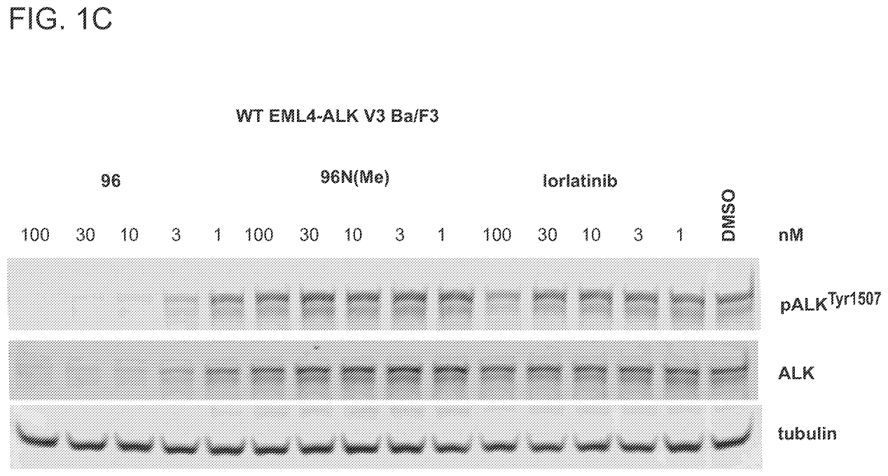

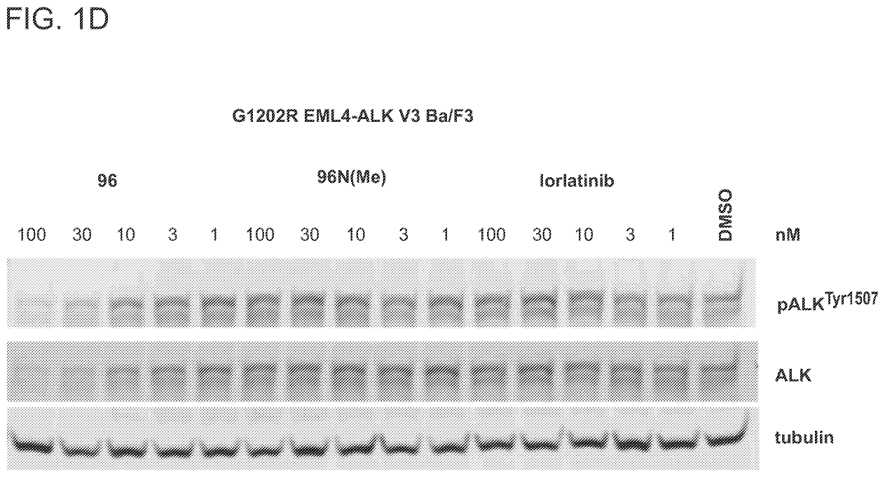

was used as a negative control. The results in FIG. 1A-FIG. 1D show that inventive bispecific compound 96 is a potent degrader of both WT ALK and G1202R ALK, exhibiting significantly improved pALK inhibition compared to Alectinib and Lorlatinib.

All patent publications and non-patent publications are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A compound of formula (I), 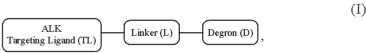
or a pharmaceutically acceptable salt or stereoisomer thereof, wherein the ALK targeting ligand is represented by formula TL-1: 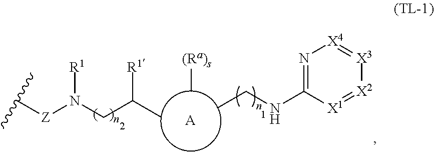
wherein
X¹ is N or CR^{b};
X² is N or CR^{c};
X³ is N or CR^{d};
X⁴ is N or CR^{e};
A is an aryl or a 5- or 6-membered heteroaryl ring which contains 1 to 4 heteroatoms selected from N, O and S(O)ᵣ;
r is 0, 1 or 2;
R^{a}, R^{b}, R^{c}, R^{d} and R^{e} are independently halo, —CN, —NO₂, —R¹, —OR², —O—NR¹R², —NR¹R², —NR¹—NR¹R², —NR¹—OR², —C(O)YR², —OC(O)YR², —NR¹C(O)YR², —SC(O)YR², —NRIC(═S)YR², —OC(═S)YR², —C(═S)YR², —YC(═NR¹)YR², —YC(═N—OR¹)YR², —YC(═N—NR¹R²)YR², —YP(═O)(YR³)(YR³), —Si(R^{3a})₃, —NR¹SO₂R², —S(O)ᵣR², —SO₂NR¹R² or —NR¹SO₂NR¹R²; wherein each Y is independently a bond, —O—, —S— or —NR¹—; or
alternatively two adjacent substituents selected from R^{b}, R^{c}, R^{d}, and R′; or two adjacent R^{a} moieties, can form with the atoms to which they are attached a 5-, 6- or 7-membered carbocyclic or heterocyclic ring, which contains 0-4 heteroatoms selected from N, O and S(O)ᵣ and which is substituted with one to four R^{f} moieties wherein,
each R^{f} moiety is independently halo, ═O, ═S, —CN, —NO₂, —R¹, —OR², —O—NR¹R², —NR¹R², —NR¹—NR¹R², —NR¹—OR², —C(O)YR², —OC(O)YR², —NR¹C(O)YR², —SC(O)YR², —NR¹C(═S)YR², —OC(═S)YR², —C(═S)YR², —YC(═NR¹)YR², —YC(═N—OR¹)YR², —YC(═N—NR¹R²)YR², —YP(═O)(YR³)(YR³), —Si(R^{3a})₃, —NR¹SO₂R², —S(O)ᵣR², —SO₂NR¹R² or —NR¹SO₂NR¹R²; or alternatively two adjacent R^{f} moieties can form with the atoms to which they are attached a 5-, 6- or 7-membered carbocyclic or heterocyclic ring, which contains 0-4 heteroatoms selected from N, O and S(O)ᵣ; provided that at least one of R^{a}, R^{b}, R^{c}, R^{d}, R^{e}, and R^{f}, when present, is or contains —P(═O)(R³)₂ or a ring system containing the moiety —P(═O)(R³)₂ as a ring member;
s is 1, 2, 3 or 4;
n₁ is 0 or 1;
n₂ is 1 or 2;
each R¹, R^{1′}, and R² is independently H, alkyl, alkenyl, alkynyl, cycloalkyl, cycloalkenyl, cycloalkynyl, aryl, heteroalkyl, heterocyclic or heteroaryl;
or R¹ and R^{1′} together with the atoms to which they are attached form a 5- to 6-membered heterocyclyl,
or R¹ and Z together with the atoms to which they are attached form a 4- to 7-membered heterocyclyl, otherwise Z is absent;
each R³ is independently H, alkyl, alkenyl, alkynyl, cycloalkyl, cycloalkenyl, cycloalkynyl, aryl, heteroalkyl, heterocyclic or heteroaryl, or two adjacent R³ moieties combine to form a ring system including a phosphorous atom;
each R^{3a} is independently H, alkyl, alkenyl, alkynyl, cycloalkyl, cycloalkenyl, cycloalkynyl, aryl, heteroalkyl, heterocyclic or heteroaryl;
alternatively, each NR¹R² moiety can be a 5-, 6- or 7-membered carbocyclic or heterocyclic ring, which can be optionally substituted and which contains 0-2 additional heteroatoms selected from N, O and S(O)ᵣ; and
each of the foregoing alkyl, alkenyl, alkynyl, cycloalkyl, cycloalkenyl, cycloalkynyl, aryl, heteroalkyl, heteroaryl and heterocyclic moieties is optionally substituted;
provided that when Rₐ is methoxy, s is not 1, or
wherein the ALK targeting ligand is represented by formula TL-1′: 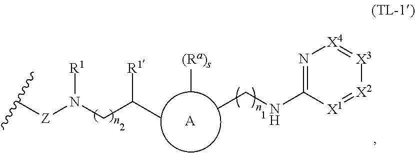
wherein
X¹ is N or CR^{b};
X² is N or CR^{c};
X³ is N or CR^{d};
X⁴ is N or CR^{e};
A is an aryl or a 5- or 6-membered heteroaryl ring which contains 1 to 4 heteroatoms selected from N, O and S(O)ᵣ;
r is 0, 1 or 2;
R^{a}, R^{b}, R^{c}, R^{d} and R^{e} are independently halo, —CN, —NO₂, —R¹, —OR², —O—NR¹R², —NR¹R², —NR¹—NR¹R², —NR¹—OR², —C(O)YR², —OC(O)YR², —NR¹C(O)YR², —SC(O)YR², —NR¹C(═S)YR², —OC(═S)YR², —C(═S)YR², —YC(═NR¹)YR², —YC(═N—OR¹)YR², —YC(═N—NR¹R²)YR², —YP(═O)(YR³)(YR³), —Si(R^{3a})₃, —NR¹SO₂R², —S(O)ᵣR², —SO₂NR¹R² or —NR¹SO₂NR¹R²; wherein each Y is independently a bond, —O—, —S— or —NR¹—; or
alternatively two adjacent substituents selected from R^{b}, R^{c}, R^{d}, and R^{e}; or two adjacent R^{a} moieties, with the atoms to which they are attached, form a 5-, 6- or 7-membered carbocyclic or heterocyclic ring, which contains 0-4 heteroatoms selected from N, O and S(O)ᵣ and which is substituted with one to four R^{f} moieties wherein,
each R^{f} moiety is independently halo, ═O, ═S, —CN, —NO₂, —R¹, —OR², —O—NR¹R², —NR¹R², —NR¹—NR¹R², —NR¹—OR², —C(O)YR², —OC(O)YR², —NR¹C(O)YR², —SC(O)YR², —NR¹C(═S)YR², —OC(═S)YR², —C(═S)YR², —YC(═NR¹)YR², —YC(═N—OR¹)YR², —YC(═N—NR¹R²)YR², —YP(═O)(YR³)(YR³), —Si(R^{3a})₃, —NR¹SO₂R², —S(O)ᵣR², —SO₂NR¹R² or —NR¹SO₂NR¹R²; or alternatively two adjacent R^{f} moieties with the atoms to which they are attached, form a 5-, 6- or 7-membered carbocyclic or heterocyclic ring, which contains 0-4 heteroatoms selected from N, O and S(O)ᵣ; provided that at least one of R^{a}, R^{b}, R^{c}, R^{d}, R^{e}, and R^{f}, when present, is or contains —P(═O)(R³)₂ or a ring system containing the moiety —P(═O)(R³)₂ as a ring member;
s is 1, 2, 3 or 4;
n₁ is 0 or 1;
n₂ is 1 or 2;
each R¹, R^{1′}, and R² is independently H, alkyl, alkenyl, alkynyl, cycloalkyl, cycloalkenyl, cycloalkynyl, aryl, heteroalkyl, heterocyclic or heteroaryl,
or R¹ and R^{1′} together with the atoms to which they are attached form a 5- to 6-membered heterocyclyl,
or R¹ and Z together with the atoms to which they are attached form a 4- to 7-membered heterocyclyl, which in some embodiments, is a bicyclic group, otherwise Z is absent;
each R³ is independently H, alkyl, alkenyl, alkynyl, cycloalkyl, cycloalkenyl, cycloalkynyl, aryl, heteroalkyl, heterocyclic or heteroaryl, or two adjacent R³ moieties combine to form a ring system including a phosphorous atom;
each R^{3a} is independently H, alkyl, alkenyl, alkynyl, cycloalkyl, cycloalkenyl, cycloalkynyl, aryl, heteroalkyl, heterocyclic or heteroaryl;
alternatively, each NR¹R² moiety independently is a 5-, 6- or 7-membered carbocyclic or heterocyclic ring, which can be optionally substituted and which contains 0-2 additional heteroatoms selected from N, O and S(O)ᵣ; and
each of the foregoing alkyl, alkenyl, alkynyl, cycloalkyl, cycloalkenyl, cycloalkynyl, aryl, heteroalkyl, heteroaryl and heterocyclic moieties is optionally substituted, or
wherein the ALK targeting ligand is represented by formula TL-2; 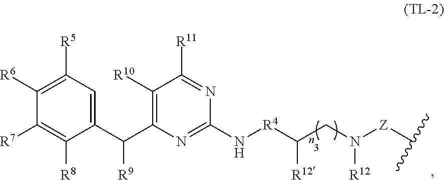
wherein
R⁴ is C₆₋₁₀ aryl, C₅₋₁₀ heteroaryl, C₃₋₁₂ cycloalkyl or C₃₋₁₀ heterocycloalkyl, wherein R⁴ is optionally substituted by R¹³, R¹⁴, R¹⁵ or R¹⁶; or wherein two adjacent substituents on R⁴ may form, together with the carbon atoms to which they are attached, a unsubstituted or substituted 5- or 6-membered carbocyclic or heterocyclic ring containing 0, 1, 2 or 3 heteroatoms selected from N, O and S;
R⁵, R⁶, R⁷, and R⁸ are independently hydrogen, C₁-C₈ alkyl, C₂-C₈ alkenyl, C₂-C₈ alkynyl, C₃-C₈ cycloalkyl, C₃-C₈ cycloalkyl-C₁-C₈ alkyl, C₅-C₁₀ aryl-C₁-C₈ alkyl, hydroxyl-C₁-C₈ alkyl, C₁-C₈ alkoxy-C₁-C₈ alkyl, amino-C₁-C₈ alkyl, halo-C₁-C₈ alkyl, unsubstituted or substituted C₅-C₁₀ aryl, unsubstituted or substituted 5 or 6 membered heterocyclyl comprising 1, 2 or 3 heteroatoms selected from N, O, and S, hydroxy, C₁-C₈ alkoxy, hydroxyl-C₁-C₈ alkoxy, C₁-C₈ alkoxy-C₁-C₈ alkoxy, halo-C₁-C₈ alkoxy, unsubstituted or substituted C₅-C₁₀ aryl-C₁-C₈ alkoxy, unsubstituted or substituted heterocyclyloxy, unsubstituted or substituted heterocyclyl-C₁-C₈ alkoxy, unsubstituted or substituted amino, C₁-C₈ alkylthio, C₁-C₈ alkylsulfinyl, C₁-C₈ alkylsulfonyl, C₅-C₁₀ arylsulfonyl, halogen, carboxy, C₁-C₈ alkoxycarbonyl, unsubstituted or substituted carbamoyl, unsubstituted or substituted sulfamoyl, cyano, nitro, —S(O)₀₋₂NR₁₉R₂₀, —S(O)₀₋₂R₂₀, —NR₁₉S(O)₀₋₂R₂₀, —C(O)NR₁₉R₂₀, —C(O)R₂₀, or —C(O)OR₂₀; wherein R₁₉ is hydrogen or C₁₋₆ alkyl; and R₂₀ is hydrogen, C₁₋₆ alkyl or C₃₋₁₂ cycloalkyl;
or R⁵ and R⁶, R⁶ and R⁷, and/or R⁷ and R⁸, together with the carbon atoms to which they are attached, form a 5- or 6-membered carbocyclic or heterocyclic ring containing 0, 1, 2 or 3 heteroatoms selected from N, O and S;
R⁹ is hydrogen or C₁₋₈ alkyl;
each R¹⁰ and R¹¹ independently is hydrogen, C₁₋₈ alkyl, C₁₋₈ alkoxy-C₁₋₈ alkyl, halo-C₁₋₈ alkyl, C₁₋₈ alkoxy, halogen, carboxy, C₁₋₈ alkoxycarbonyl, unsubstituted or substituted carbamoyl, cyano, or nitro;
R¹² and R^{12′} independently are hydrogen or C₁₋₆ alkyl,
or R¹² and R^{12′} together with the atoms to which they are attached form a 5- to 6-membered heterocyclyl,
or R¹² and Z together with the atoms to which they are attached form a 4- to 7-membered heterocyclyl, otherwise Z is absent;
R¹³, R¹⁴, R¹⁵ and R¹⁶ are independently hydrogen, C₁-C₈ alkyl, C₂-C₈ alkenyl, C₂-C₈ alkynyl, C₃-C₈ cycloalkyl, C₃-C₈ cycloalkyl-C₁-C₈ alkyl, C₅-C₁₀ aryl-C₁-C₈ alkyl, hydroxyl-C₁-C₈ alkyl, C₁-C₈ alkoxy-C₁-C₈ alkyl, amino-C₁-C₈ alkyl, halo-C₁-C₈ alkyl, unsubstituted or substituted C₅-C₁₀ aryl, unsubstituted or substituted 5 or 6 membered heterocyclyl containing 1, 2 or 3 heteroatoms selected from N, O, and S, hydroxy, C₁-C₂ alkoxy, hydroxyl-C₁-C₈ alkoxy, C₁-C₈ alkoxy-C₁-C₈ alkoxy, halo-C₁-C₈ alkoxy, unsubstituted or substituted C₅-C₁₀ aryl-C₁-C₈ alkoxy, unsubstituted or substituted heterocyclyloxy, unsubstituted or substituted heterocyclyl-C₁-C₈ alkoxy, unsubstituted or substituted amino, C₁-C₈ alkylthio, C₁-C₈ alkylsulfinyl, C₁-C₈ alkylsulfonyl, C₅-C₁₀ arylsulfonyl, halogen, carboxy, C₁-C₈ alkoxycarbonyl, unsubstituted or substituted carbamoyl, unsubstituted or substituted sulfamoyl, cyano, nitro, —S(O)₀₋₂NR₁₉R₂₀, —S(O)₀₋₂R₁₉, —C(O)R₁₈, —CXR₁₈, —NR₁₉XR₁₈, —NR₁₉XNR₁₉R₂₀, —OXNR₁₉R₂₀, —OXOR₁₉, or —XR₁₈;
X is a bond or C₁₋₆ alkylene;
R¹⁸ is independently C₆₋₁₀ aryl, C₅₋₁₀ heteroaryl, C₃₋₁₂ cycloalkyl or C₃₋₁₀ heterocycloalkyl;
R₁₉ and R₂₀ are independently hydrogen or C₁₋₆ alkyl;
n₃ is 1 or 2;
and any aryl, heteroaryl, cycloalkyl, or heterocycloalkyl of R¹⁸ is optionally substituted by 1 to 3 radicals independently selected from C₁₋₆ alkyl, C₃₋₁₀ heterocycloalkyl-C₀₋₄ alkyl optionally substituted with C₁₋₆ alkyl, —C(O)R₁₉, —C(O)N₁₉R₂₀, —XNR₁₉R₂₀, —NR₁₉XNR₁₉R₂₀, and —NR₁₉C(O)R₂₀; wherein X is a bond or C₁₋₆ alkylene, or
wherein the ALK targeting ligand is represented by formula TL-3: 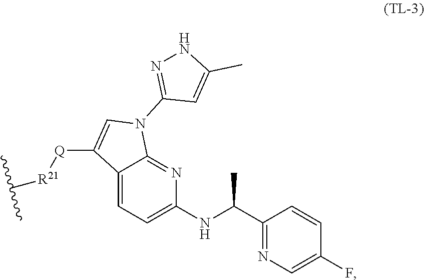
wherein
R²¹ is C₁-C₈ alkyl, C₂-C₈ alkenyl, C₂-C₈ alkynyl, C₃-C₈ cycloalkyl, C₃-C₈ cycloalkyl-C₁-C₈ alkyl, C₅-C₁₀ aryl-C₁-C₈ alkyl, hydroxyl-C₁-C₈ alkyl, C₁-C₈ alkoxy-C₁-C₈ alkyl, halo-C₁-C₈ alkyl, unsubstituted or substituted amino, unsubstituted or substituted C₅-C₁₀ aryl, unsubstituted or substituted 5- or 6-membered heterocyclyl containing 1, 2 or 3 heteroatoms selected from N, O, and S; and
Q is CH₂ or C(O):
the linker represents a moiety that connects covalently the degron and the targeting ligand; and
wherein the degron binds cereblon, von Hippel Landau tumor suppressor (VHL), inhibitor of apoptosis protein (IAP), or murine double minute 2 (MDM2).

2. The compound of claim 1, wherein the ALK targeting ligand is of formula TL-1.

3. The compound of claim 2, wherein Z is absent, n₁ is 0, n₂ is 1, R^{1′} is H, X² is CR^{c} and R^{c} is 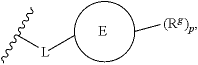
and the ALK targeting ligand has a structure represented by formula TL-1a: 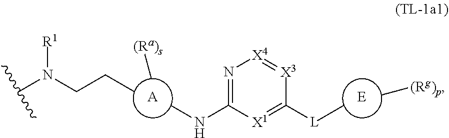
wherein
E is an aryl or a 5- or 6-membered heteroaryl ring which contains 1 to 4 heteroatoms selected from N, O and S(O)ᵣ;
r is 0, 1 or 2;
each R^{g} is independently halo, —CN, —NO₂, —R¹, —OR², —O—NR¹R², —NR¹R², —NR¹—NR¹R², —NR¹—OR², —C(O)YR², —OC(O)YR², —NR¹C(O)YR², —SC(O)YR², —NR¹C(═S)YR², —OC(═S)YR², —C(═S)YR², —YC(═NR¹)YR², —YC(═N—OR¹)YR², —YC(═N—NR¹R²)YR², —YP(═O)(YR³)(YR³), —Si(R^{3a})₃, —NR¹SO₂R², —S(O)ᵣR², —SO₂NR¹R² or —NR¹SO₂NR¹R²; or alternatively, R^{g} may also be or include an independently selected moiety,
—P(═O)(R³)₂ or a ring system containing the moiety —P(═O)(R³)₂ as a ring member;
at least one of R^{a} and R^{g} is or contains —P(═O)(R³)₂ or a ring system containing the moiety —P(═O)(R³)₂ as a ring member;
L is O or NH; and
p is 1, 2, 3 or 4, or
wherein Z is absent, n₁ is 0, n₂ is 2, R¹ and R^{1′} together with the atoms to which they are attached form a piperidinyl ring, X² is CR^{c} and R^{c} is 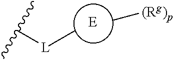
and the ALK targeting ligand has a structure represented by formula TL-1a2: 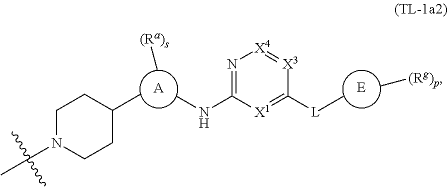
wherein
E is an aryl or a 5- or 6-membered heteroaryl ring which contains 1 to 4 heteroatoms selected from N, O and S(O)ᵣ;
r is 0, 1 or 2;
each R^{g} is independently halo, —CN, —NO₂, —R¹, —OR², —O—NR¹R², —NR¹R², —NR¹—NR¹R², —NR¹—OR², —C(O)YR², —OC(O)YR², —NR¹C(O)YR², —SC(O)YR², —NR¹C(═S)YR², —OC(═S)YR², —C(═S)YR², —YC(═NR¹)YR², —YC(═N—OR¹)YR², —YC(═N—NR¹R²)YR², —YP(═O)(YR³)(YR³), —Si(R^{3a})₃, —NR¹SO₂R², —S(O)ᵣR², —SO₂NR¹R² or —NR¹SO₂NR¹R²; or alternatively, R^{g} may also be or include an independently selected moiety, —P(═O)(R³)₂ or a ring system containing the moiety —P(═O)(R³)₂ as a ring member;
at least one of R^{a} and R^{g} is or contains —P(═O)(R³)₂ or a ring system containing the moiety —P(═O)(R³)₂ as a ring member;
L is O or NH; and
p is 1, 2, 3 or 4.

4. The compound of claim 3, wherein A and E are each phenyl and the ALK targeting ligand has a structure represented by formula TL-1a1a:

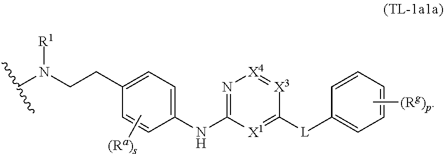

5. The compound of claim 4, wherein X¹ is N, X³ is C—Cl, X⁴ is CH, L is NH, R¹ is H, R^{a} is independently Me or OMe, s is 2, R^{g} is —P(═O)(Me)₂ and p is 1, and the ALK targeting ligand has a structure represented by formula TL-1a1a1: 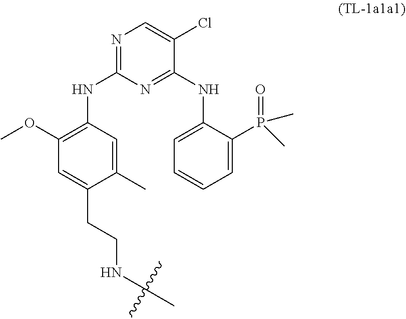
or
wherein X¹ is N, X³ is C—Cl, X⁴ is CH, L is NH, R¹ is Me, R^{a} is independently Me or OMe, s is 2, R^{g} is —P(═O)(Me)₂ and p is 1, and the ALK targeting ligand has a structure represented by formula TL-1a1a2:

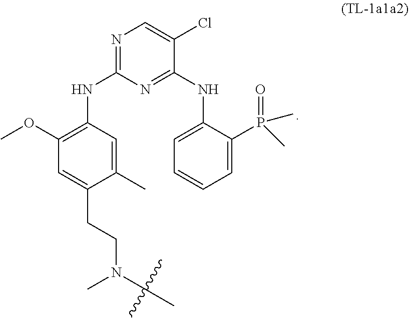

6. (canceled)

7. (canceled)

8. The compound of claim 3, wherein A and E are each phenyl and the ALK targeting ligand has a structure represented by formula TL-1a2a:

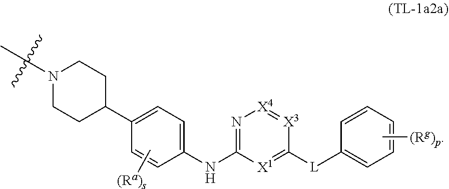

9. The compound of claim 8, wherein X¹ is N, X³ is C—Cl, X⁴ is CH, L is NH, R^{a} is independently Me or OMe, s is 2, R^{g} is —P(═O)(Me)₂ and p is 1, and the ALK targeting ligand has a structure represented by formula TL-1a2a1:

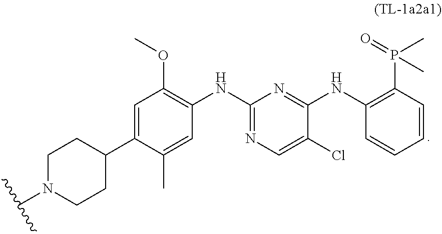 [SMILES: *N1CCC(c2cc(OC)c(Nc3ncc(Cl)c(Nc4ccccc4P(C)(C)=O)n3)cc2C)CC1]

10. The compound of claim 1, wherein the ALK targeting ligand is of formula TL-1′.

11. The compound of claim 10, wherein Z is absent and the ALK targeting ligand has a structure represented by formula TL-1′a:

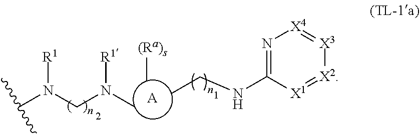

12. The compound of claim 11, wherein n₁ is 0, n₂ is 2, R¹ and R^{1′} together with the atoms to which they are attached form piperazinyl, X² is CR^{c} wherein R^{c} is 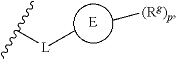
and the ALK targeting ligand has a structure represented by formula TL-1′a1: 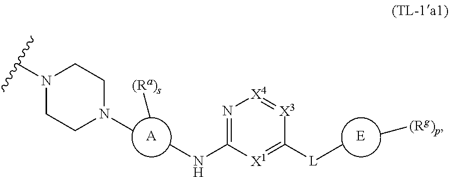
wherein
E is an aryl or a 5- or 6-membered heteroaryl ring which contains 1 to 4 heteroatoms selected from N, O and S(O)ᵣ;
r is 0, 1 or 2;
each R^{g} is independently halo, —CN, —NO₂, —R¹, —OR², —O—NR¹R², —NR¹R², —NR¹—NR¹R², —NR¹—OR², —C(O)YR², —OC(O)YR², —NR¹C(O)YR², —SC(O)YR², —NR¹C(═S)YR², —OC(═S)YR², —C(═S)YR², —YC(═NR¹)YR², —YC(═N—OR¹)YR², —YC(═N—NR¹R²)YR², —YP(═O)(YR³)(YR³), —Si(R^{3a})₃, —NR¹SO₂R², —S(O)ᵣR², —SO₂NR¹R² or —NR¹SO₂NR¹R²; or alternatively, each R^{g} may also be or include an independently selected moiety, —P(═O)(R³)₂ or a ring system containing the moiety —P(═O)(R³)₂ as a ring member;
at least one of R^{a} and R^{g} is or contains —P(═O)(R³)₂ or a ring system containing the moiety
—P(═O)(R³)₂ as a ring member;
L is O or NH; and
p is 1, 2, 3 or 4

13. The compound of claim 12, wherein A and E are each phenyl and the ALK targeting ligand has a structure represented by formula TL-1′a1a:

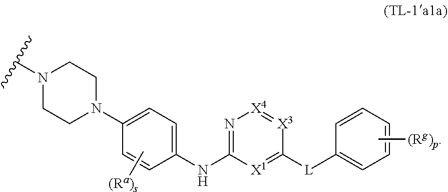

14. The compound of claim 13, wherein X¹ is N, X³ is C—Cl, X⁴ is CH, L is NH, R^{a} is OMe, s is 1, R^{g} is —P(═O)(Me)₂ and p is 1, and the ALK targeting ligand has a structure represented by formula TL-1′a1a1:

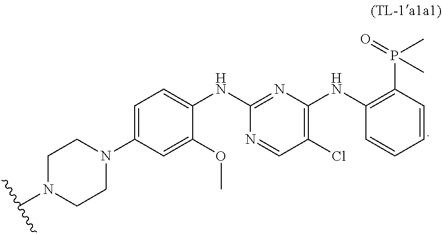 [SMILES: *N1CCN(c2ccc(Nc3ncc(Cl)c(Nc4ccccc4P(C)(C)=O)n3)c(OC)c2)CC1]

15. The compound of claim 1, wherein the ALK targeting ligand is of formula TL-2.

16. The compound of claim 15, wherein Z is absent, R⁴ is aryl optionally substituted with R¹⁷, and the ALK targeting ligand has a structure represented by formula TL-2a1: 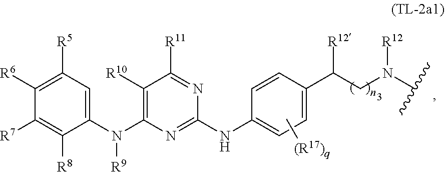
wherein
R¹⁷ is independently hydrogen, C₁-C₈ alkyl, C₂-C₈ alkenyl, C₂-C₈ alkynyl, C₃-C₈ cycloalkyl, C₃-C₈ cycloalkyl-C₁-C₈ alkyl, C₅-C₁₀ aryl-C₁-C₈ alkyl, hydroxyl-C₁-C₈ alkyl, C₁-C₈ alkoxy-C₁-C₈ alkyl, amino-C₁-C₈ alkyl, halo-C₁-C₈ alkyl, unsubstituted or substituted C₅-C₁₀ aryl, unsubstituted or substituted 5- or 6-membered heterocyclyl containing 1, 2 or 3 heteroatoms selected from N, O, and S, hydroxy, C₁-C₂ alkoxy, hydroxyl-C₁-C₈ alkoxy, C₁-C₈ alkoxy-C₁-C₈ alkoxy, halo-C₁-C₈ alkoxy, unsubstituted or substituted C₅-C₁₀ aryl-C₁-C₈ alkoxy, unsubstituted or substituted heterocyclyloxy, unsubstituted or substituted heterocyclyl-C₁-C₈ alkoxy, unsubstituted or substituted amino, C₁-C₈ alkylthio, C₁-C₈ alkylsulfinyl, C₁-C₈ alkylsulfonyl, C₅-C₁₀ arylsulfonyl, halogen, carboxy, C₁-C₈ alkoxycarbonyl, unsubstituted or substituted carbamoyl, unsubstituted or substituted sulfamoyl, cyano, nitro, —S(O)₀₋₂NR₁₉R₂₀, —S(O)₀₋₂R₁₉, —C(O)R₁₈, —CXR₁₈, —NR₁₉XR₁₈, —NR₁₉XNR₁₉R₂₀, —OXNR₁₉R₂₀, —OXOR₁₉, or —XR₁₈; and
q is 0, 1 or 2.

17. The compound of claim 16, wherein n₃ is 1, R⁵ is H, R⁶ is H, R⁷ is H, R⁸ is SO₂iPr, R⁹ is H, R¹⁰ is Cl, R¹¹ is H, R¹² is H, R^{12′} is H, R¹⁷ is independently Me or OMe, and q is 2, and the ALK targeting ligand has a structure represented by formula TL-2a1a: 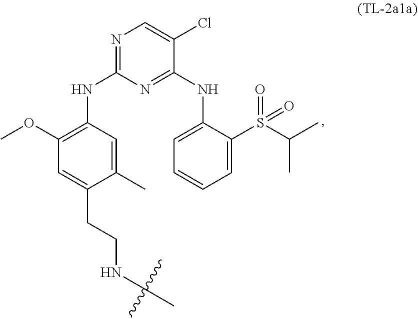 [SMILES: *NCCc1cc(OC)c(Nc2ncc(Cl)c(Nc3ccccc3S(=O)(=O)C(C)C)n2)cc1C]
or
wherein n₃ is 1, R⁵ is H, R⁶ is H, R⁷ is H, R⁸ is SO₂iPr, R⁹ is H, R¹⁰ is Cl, R¹¹ is H, R¹² is Me, R^{12′} is H, R¹⁷ is independently Me or OMe, and q is 2, and the ALK targeting ligand has a structure represented by formula TL-2a1b: 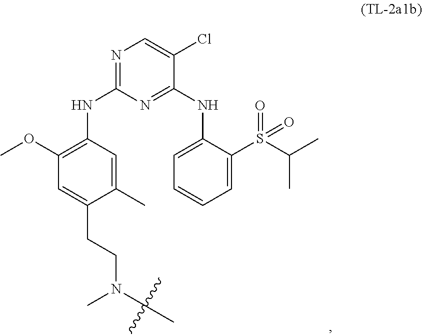 [SMILES: *N(C)CCc1cc(OC)c(Nc2ncc(Cl)c(Nc3ccccc3S(=O)(=O)C(C)C)n2)cc1C]
or
wherein n₃ is 2, R⁵ is H, R⁶ is H, R⁷ is H, R⁸ is SO₂iPr, R⁹ is H, R¹⁰ is Cl, R¹¹ is H, R¹² and R^{12′} together with the atoms to which they are attached form a piperidinyl ring, R¹⁷ is independently Me or OMe, and q is 2, and the ALK targeting ligand has a structure represented by formula TL-2a1c: 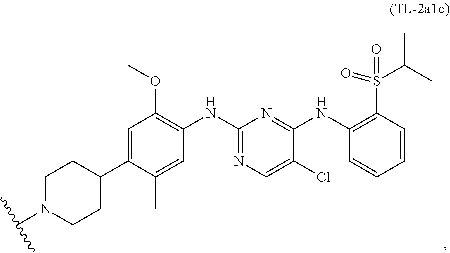 [SMILES: *N1CCC(c2cc(OC)c(Nc3ncc(Cl)c(Nc4ccccc4S(=O)(=O)C(C)C)n3)cc2C)CC1]
or
wherein n₃ is 1, R⁵ is H, R⁶ is H, R⁷ is H, R⁸ is C(O)NHMe, R⁹ is H, R¹⁰ is Cl, R¹¹ is H, R¹² is Me, R^{12′} is H, R¹⁷ is Me or OMe, and q is 2, and the ALK targeting ligand has a structure represented by formula TL-2a1d: 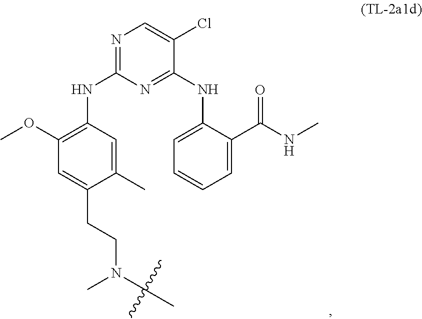
or
wherein n₃ is 2, R⁵ is H, R⁶ is H, R⁷ is H, R⁸ is SO₂iPr, R⁹ is H, R¹⁰ is Cl, R¹¹ is H, R¹² and R^{12′} together with the atoms to which they are attached form a piperidinyl ring, R¹⁷ is independently Me or OEt, and q is 2, and the ALK targeting ligand has a structure represented by formula TL-2a1e:

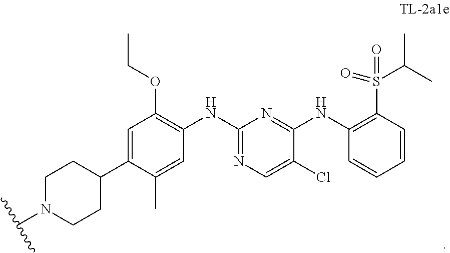 [SMILES: *N1CCC(c2cc(OCC)c(Nc3ncc(Cl)c(Nc4ccccc4S(=O)(=O)C(C)C)n3)cc2C)CC1]

18.-21. (canceled)

22. The compound of claim 15, wherein n₃ is 1, R⁵ is H, R⁶ is H, R⁷ is H, R⁸ is SO₂iPr, R⁹ is H, R¹⁰ is Cl, R¹¹ is H, R^{12′} is H, R¹² and Z together with the atoms to which they are attached form 2,6-diazospiro[3.3]heptane, R¹⁷ is independently Me or OMe, and q is 2, and the ALK targeting ligand has a structure represented by formula TL-2b1a: 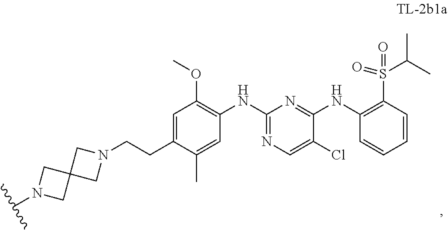 [SMILES: *N1CC2(C1)CN(CCc1cc(OC)c(Nc3ncc(Cl)c(Nc4ccccc4S(=O)(=O)C(C)C)n3)cc1C)C2]
or
wherein n₃ is 1, R⁵ is H, R⁶ is H, R⁷ is H, R⁸ is SO₂iPr, R⁹ is H, R¹⁰ is Cl, R¹¹ is H, R^{12′} is H, R¹² and Z, together with the atoms to which they are attached, form piperidinyl, R¹⁷ is independently Me or OMe, and q is 2, and the ALK targeting ligand has a structure represented by formula TL-2b1b: 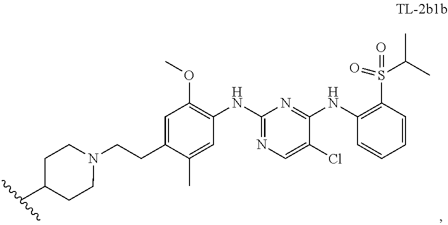 [SMILES: *C1CCN(CCc2cc(OC)c(Nc3ncc(Cl)c(Nc4ccccc4S(=O)(=O)C(C)C)n3)cc2C)CC1]
or
wherein n₃ is 1, R⁵ is H, R⁶ is H, R⁷ is H, R⁸ is SO₂iPr, R⁹ is H, R¹⁰ is Cl, R¹¹ is H, R^{12′} is H, R¹² and Z, together with the atoms to which they are attached, form piperazinyl, R¹⁷ is independently Me or OMe, and q is 2, and the ALK targeting ligand has a structure represented by formula TL-2b1c:

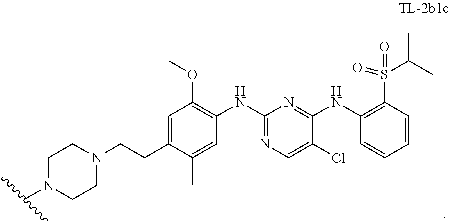 [SMILES: *N1CCN(CCc2cc(OC)c(Nc3ncc(Cl)c(Nc4ccccc4S(=O)(=O)C(C)C)n3)cc2C)CC1]

23. (canceled)

24. (canceled)

25. The compound of claim 1, wherein the ALK targeting ligand is of formula TL-3.

26. The compound of claim 25, wherein Q is C(O) and R²¹ is 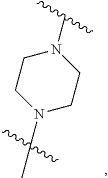 [SMILES: *N1CCN(*)CC1]
and the [6-{[(1S)-1-(5-fluoropyridin-2-yl)ethyl]amino}-1-(5-methyl-1H-pyrazol-3-yl)-1H-pyrrolo[2,3-b]pyridine analog has a structure represented by formula TL-3a: 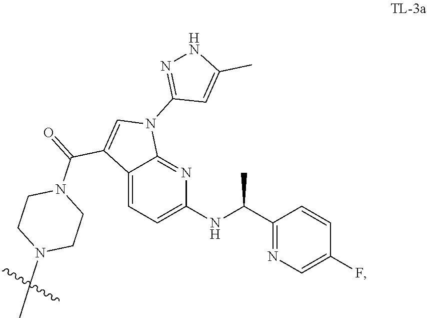 [SMILES: *N1CCN(C(=O)c2cn(-c3cc(C)[nH]n3)c3nc(N[C@@H](C)c4ccc(F)cn4)ccc23)CC1]
or
wherein Q is C(O) and R²¹ is NMe, and the [6-{[(1S)-1-(5-fluoropyridin-2-yl)ethyl]amino}-1-(5-methyl-1H-pyrazol-3-yl)-1H-pyrrolo[2,3-b]pyridine analog has a structure represented by formula TL-3b:

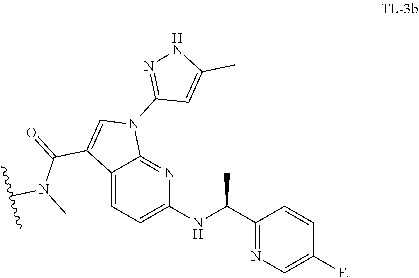 [SMILES: *N(C)C(=O)c1cn(-c2cc(C)[nH]n2)c2nc(N[C@@H](C)c3ccc(F)cn3)ccc12]

27. (canceled)

28. The compound of claim 1, wherein the linker is represented by any one of the following structures:

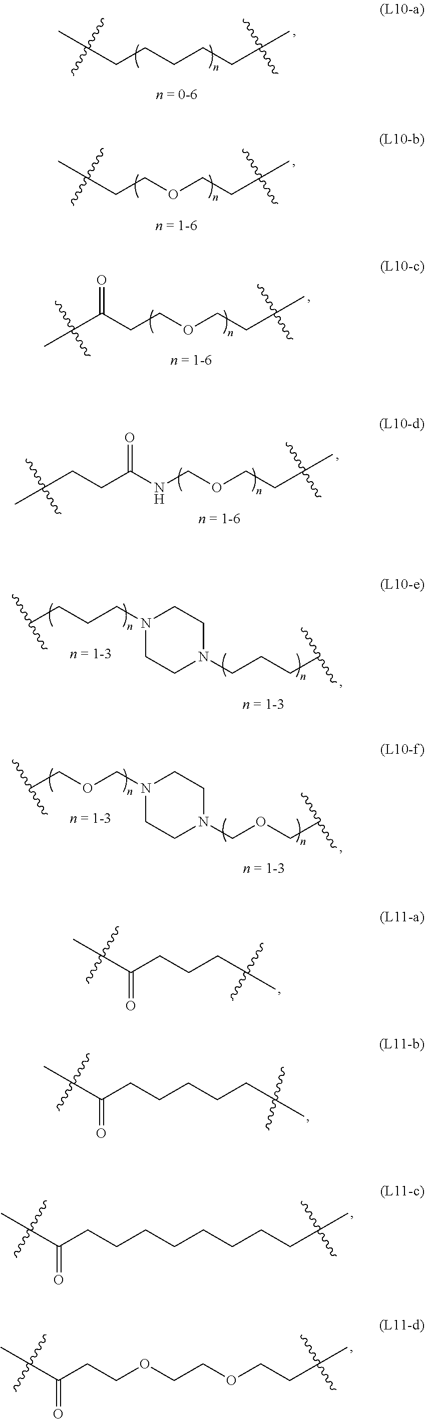

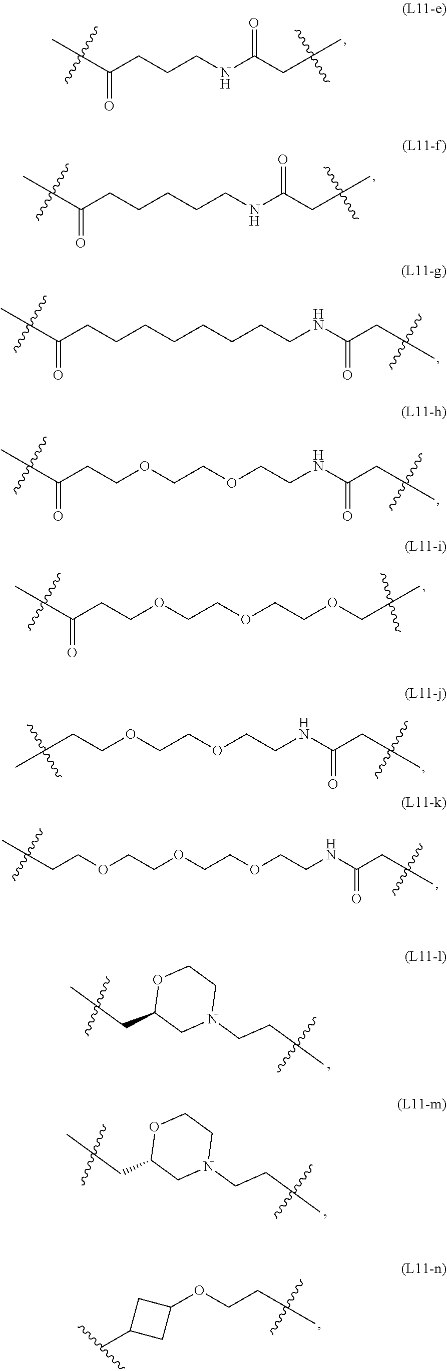

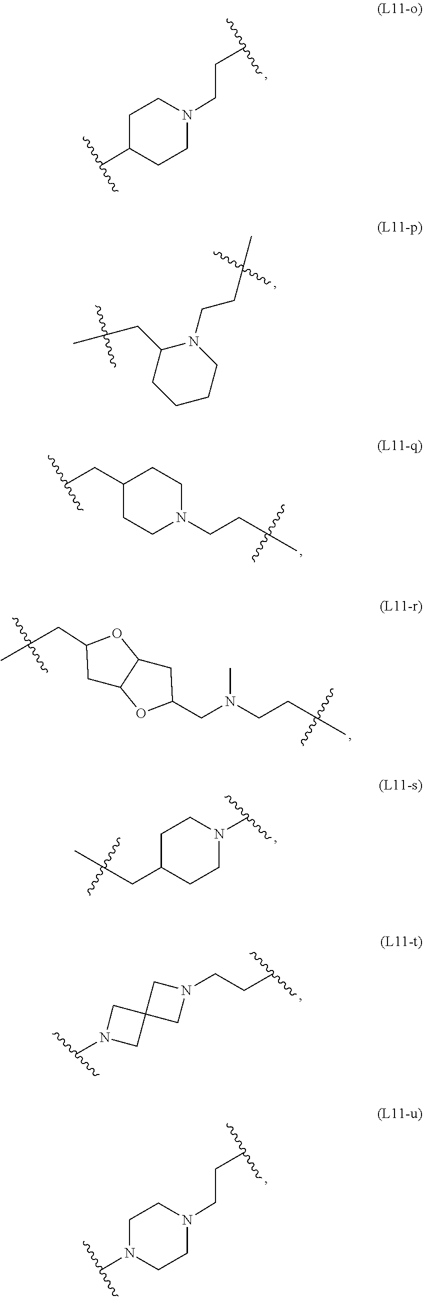

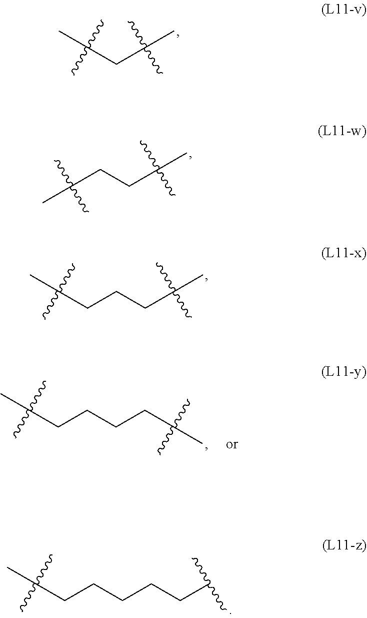

29. The compound of claim 1, which is represented by any one of the following structures: 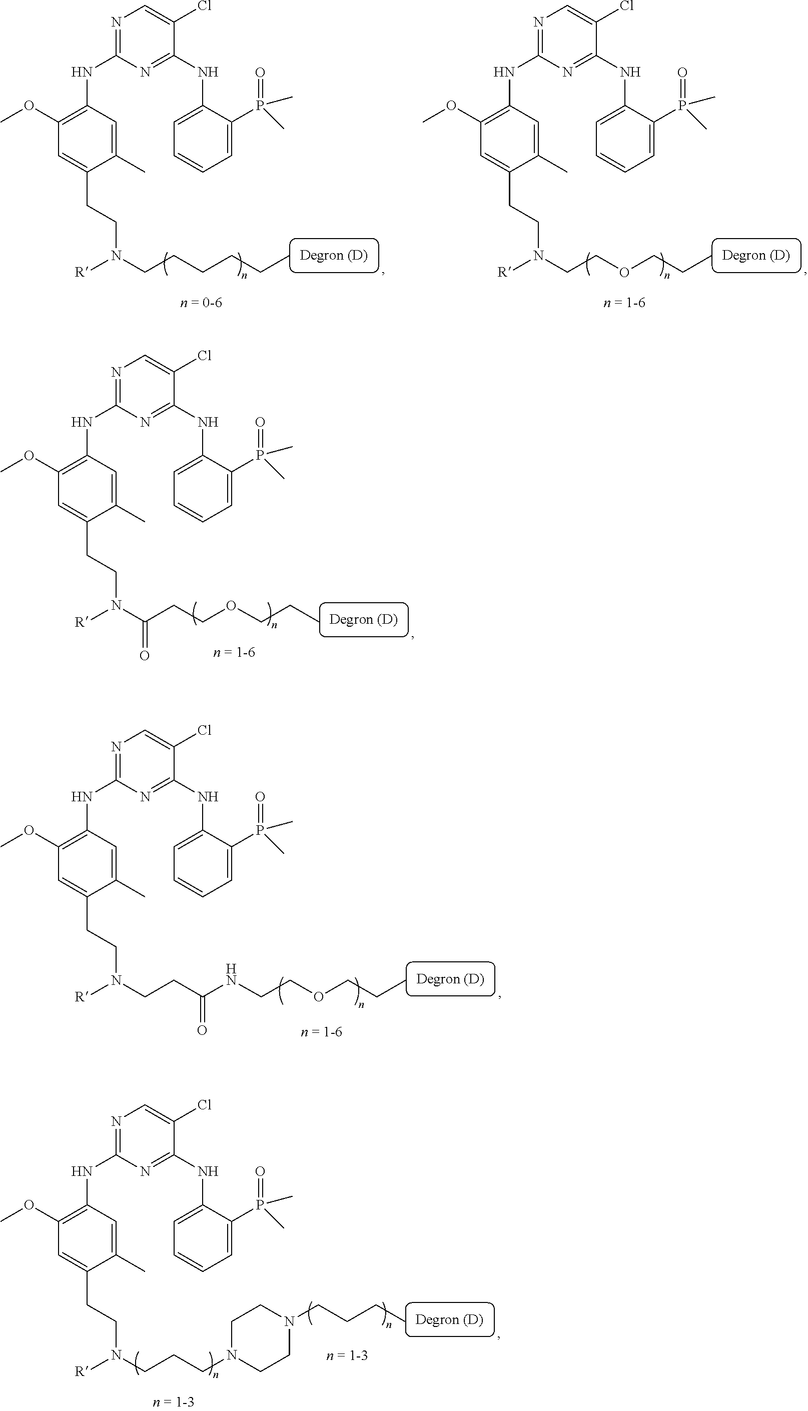 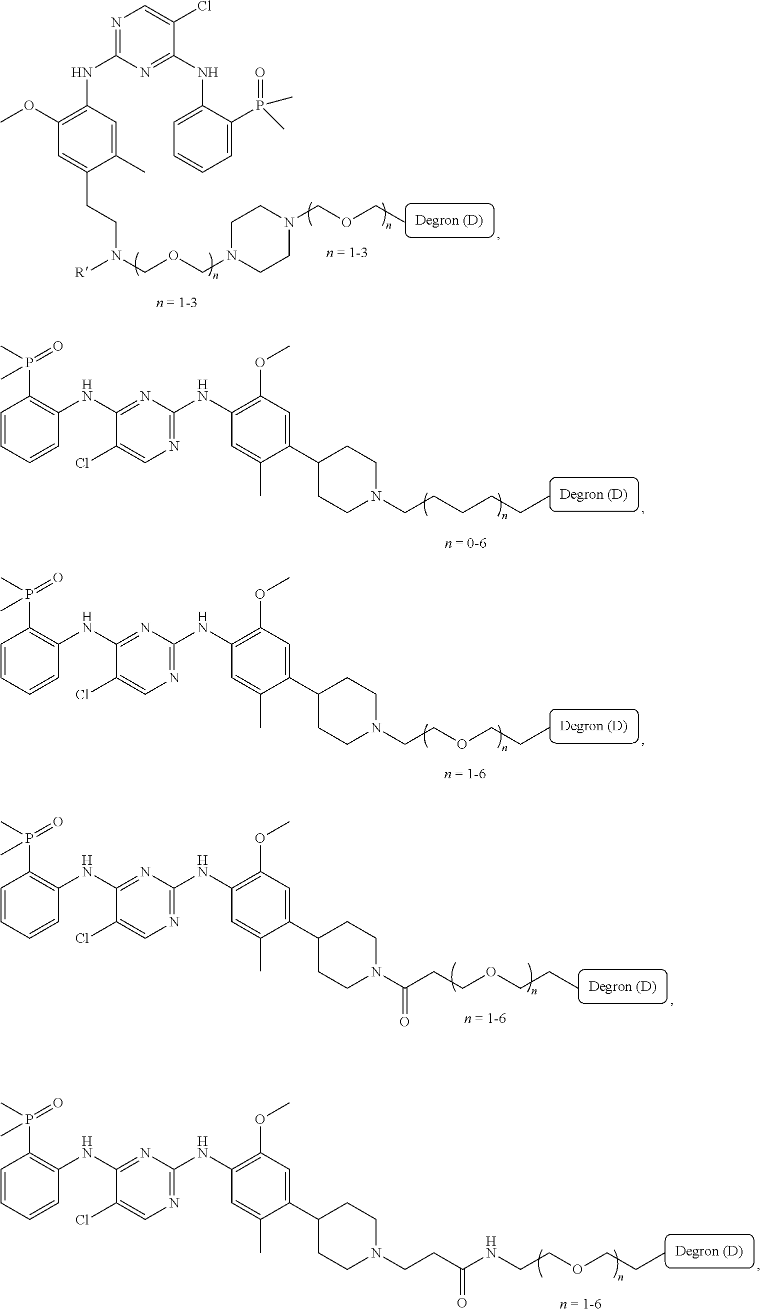 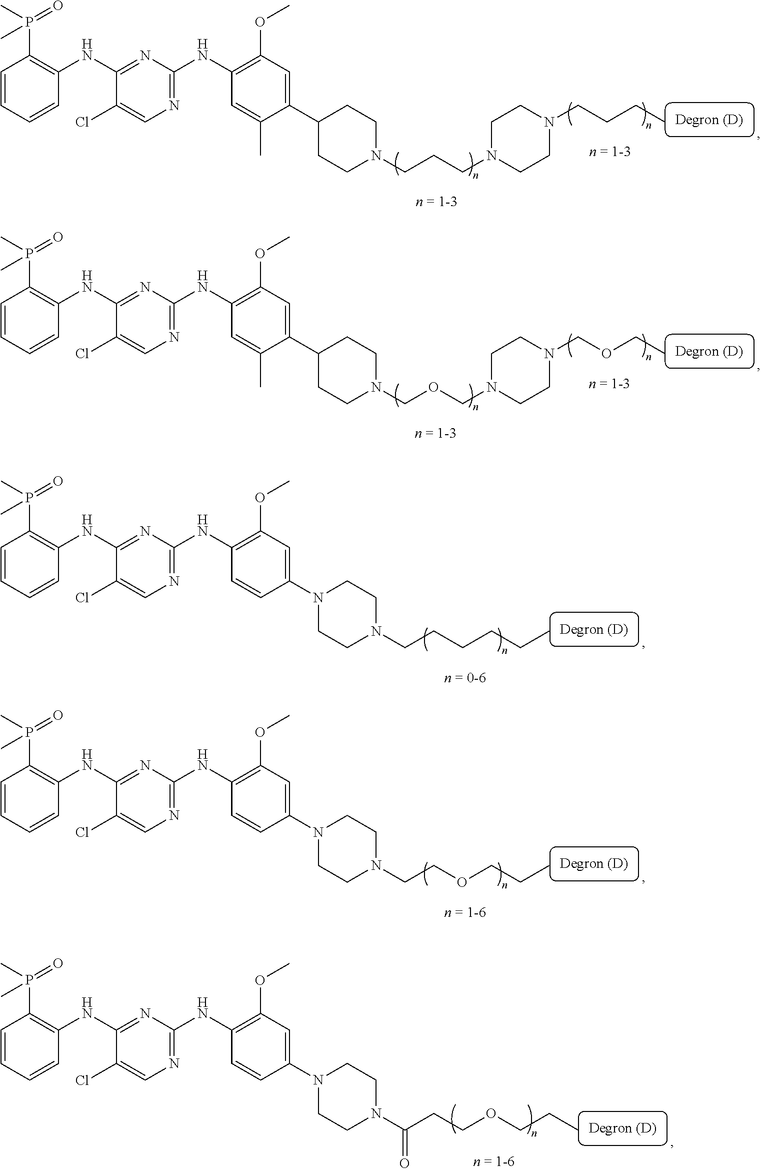 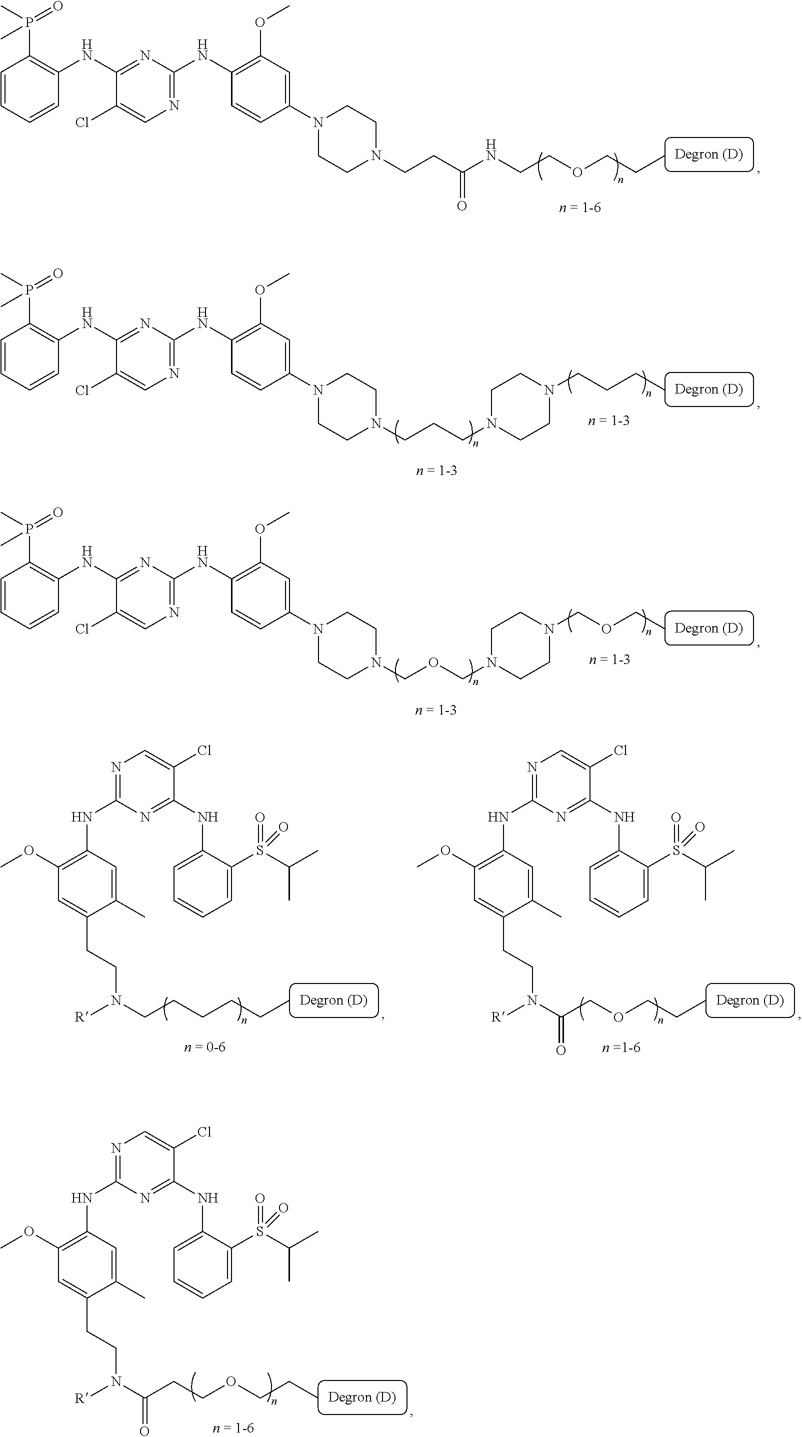 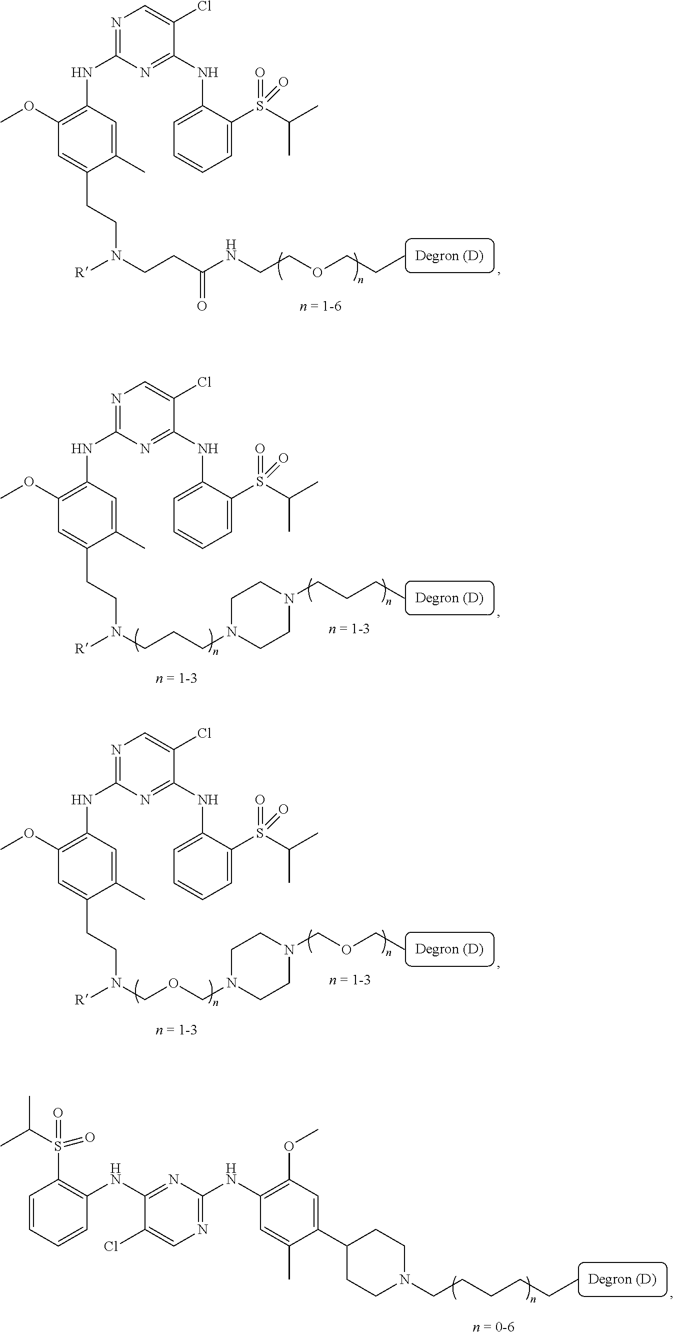 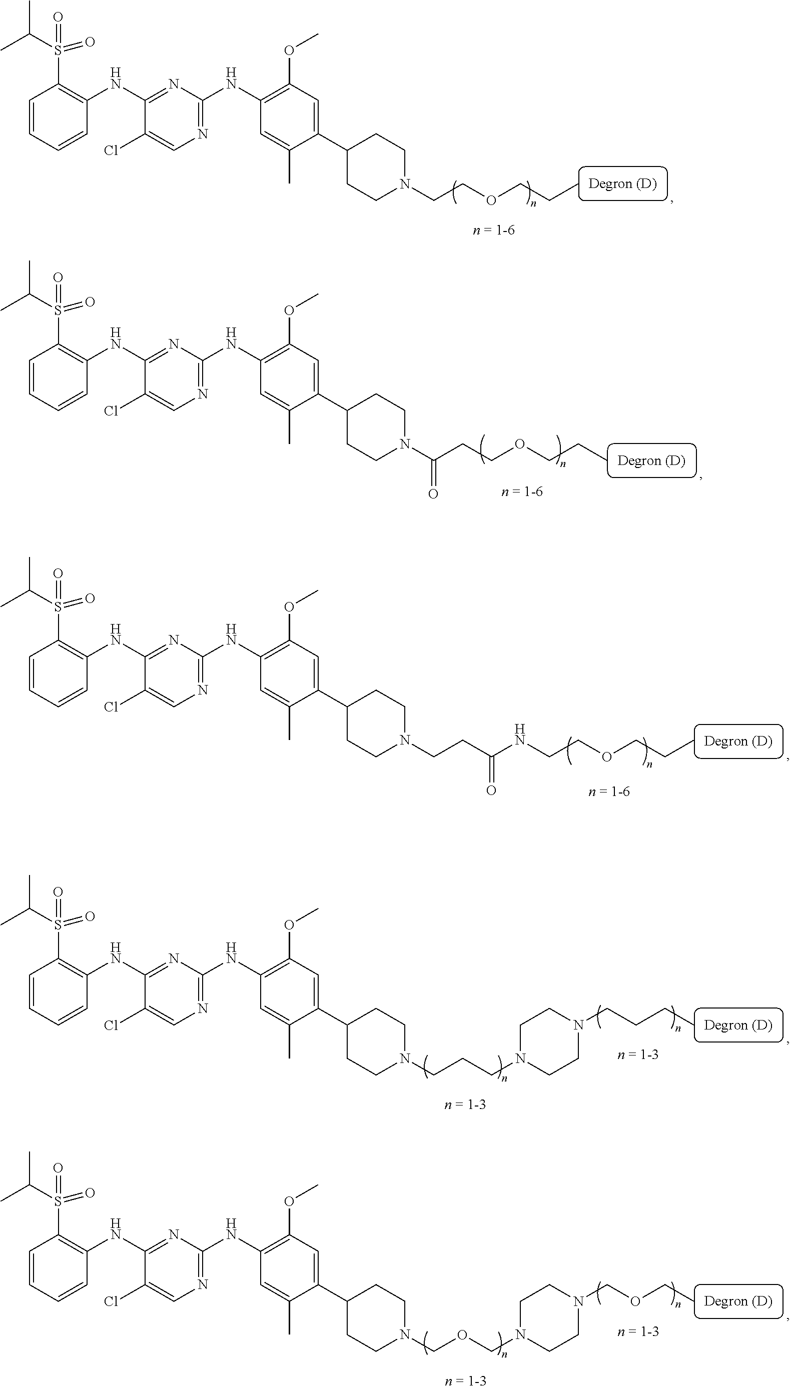 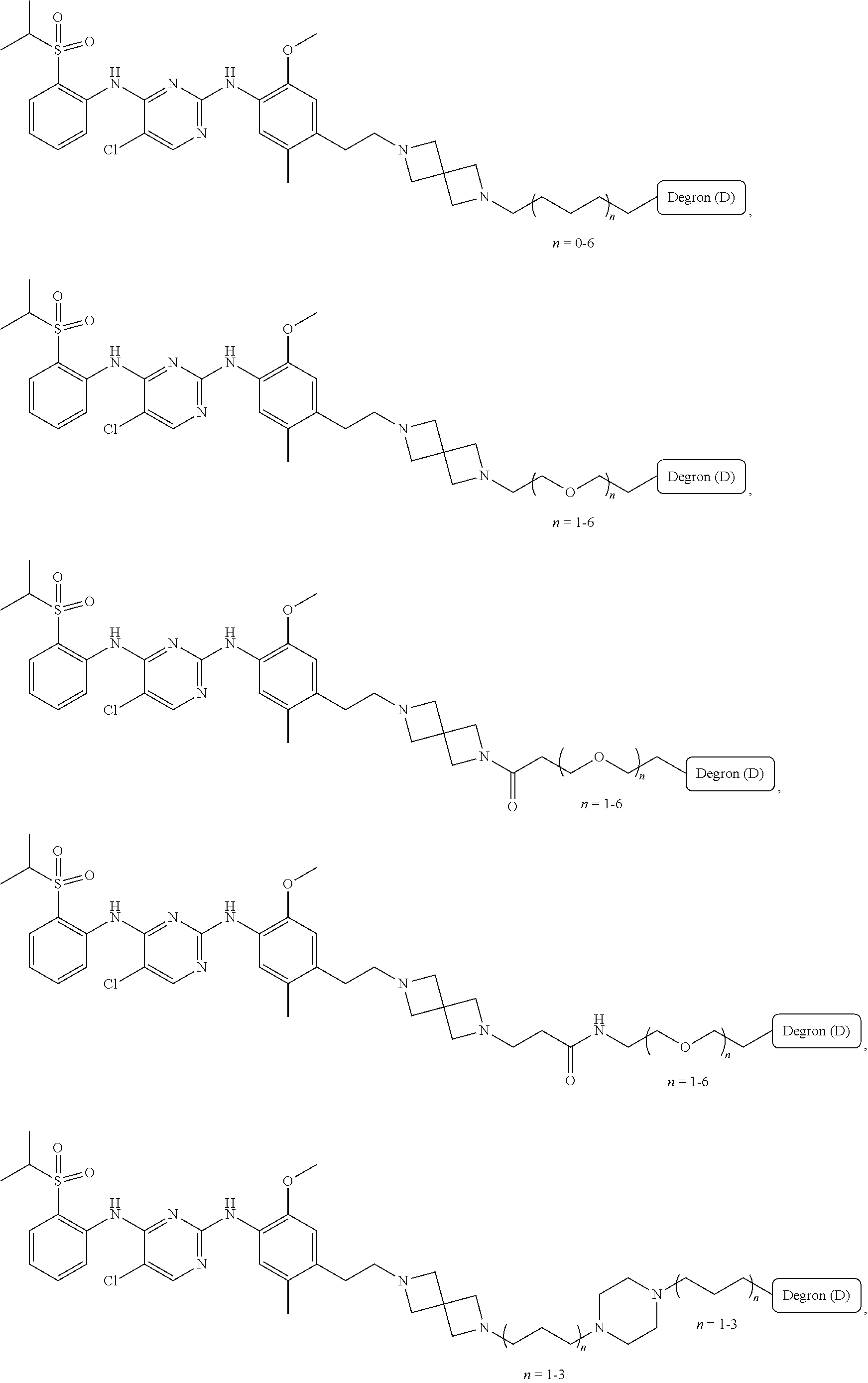 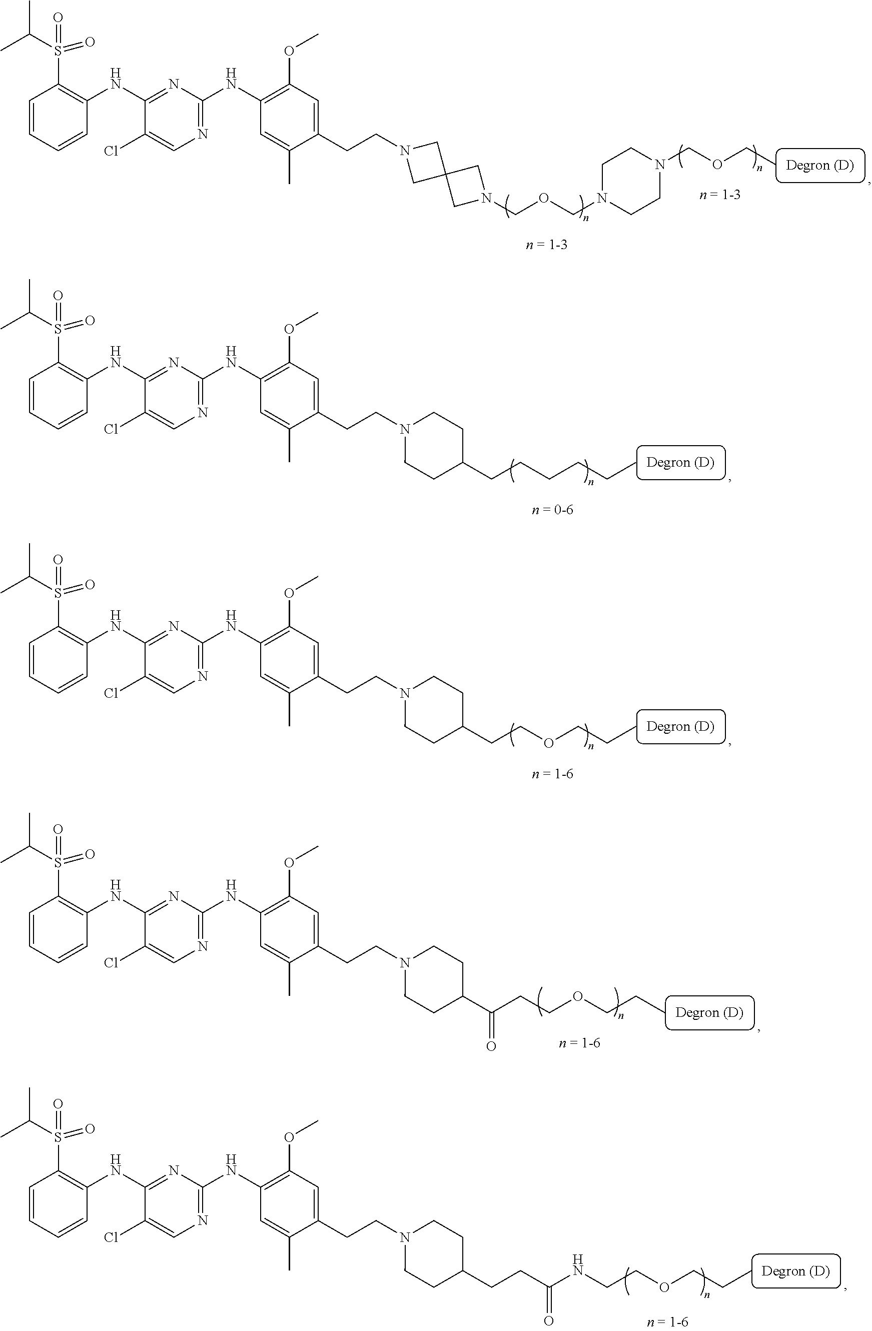 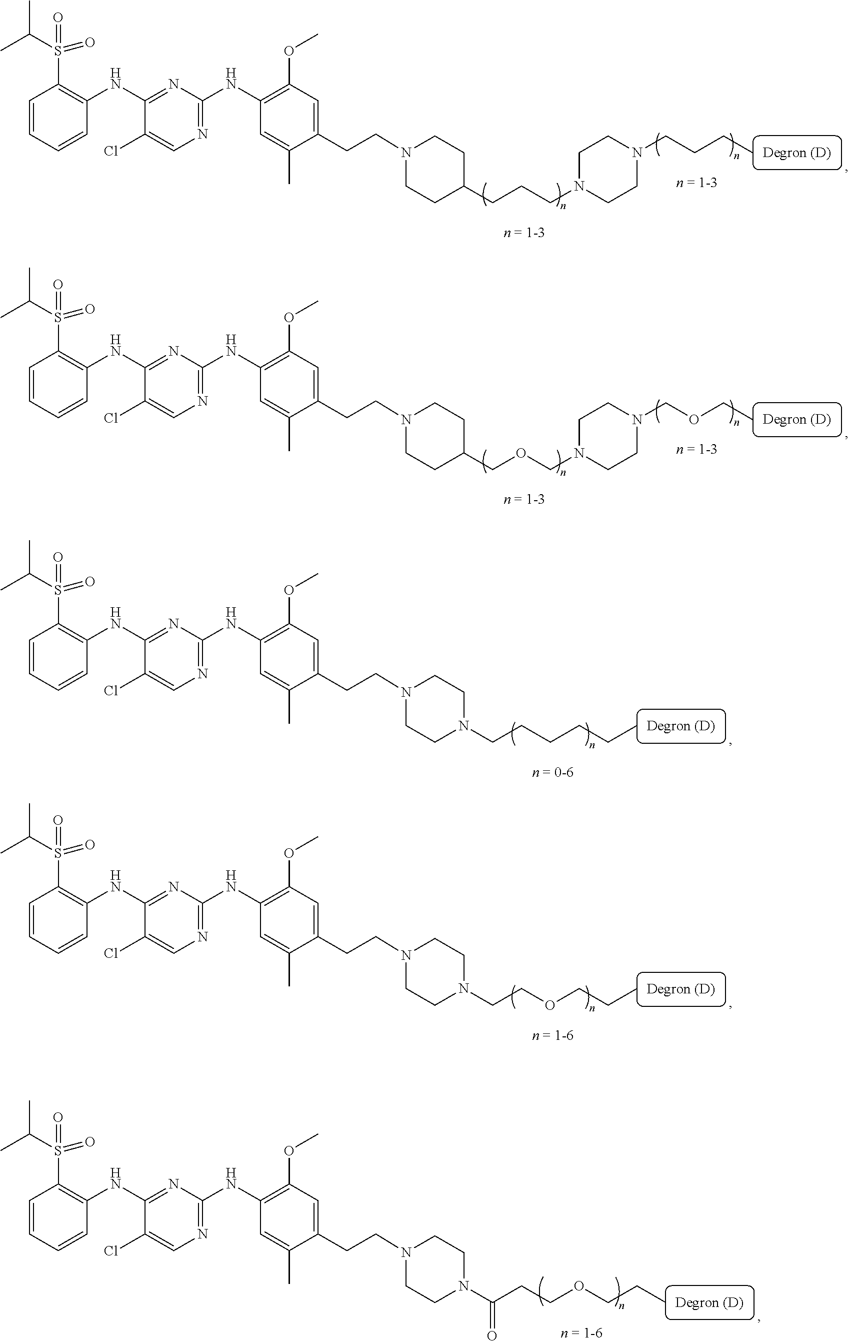 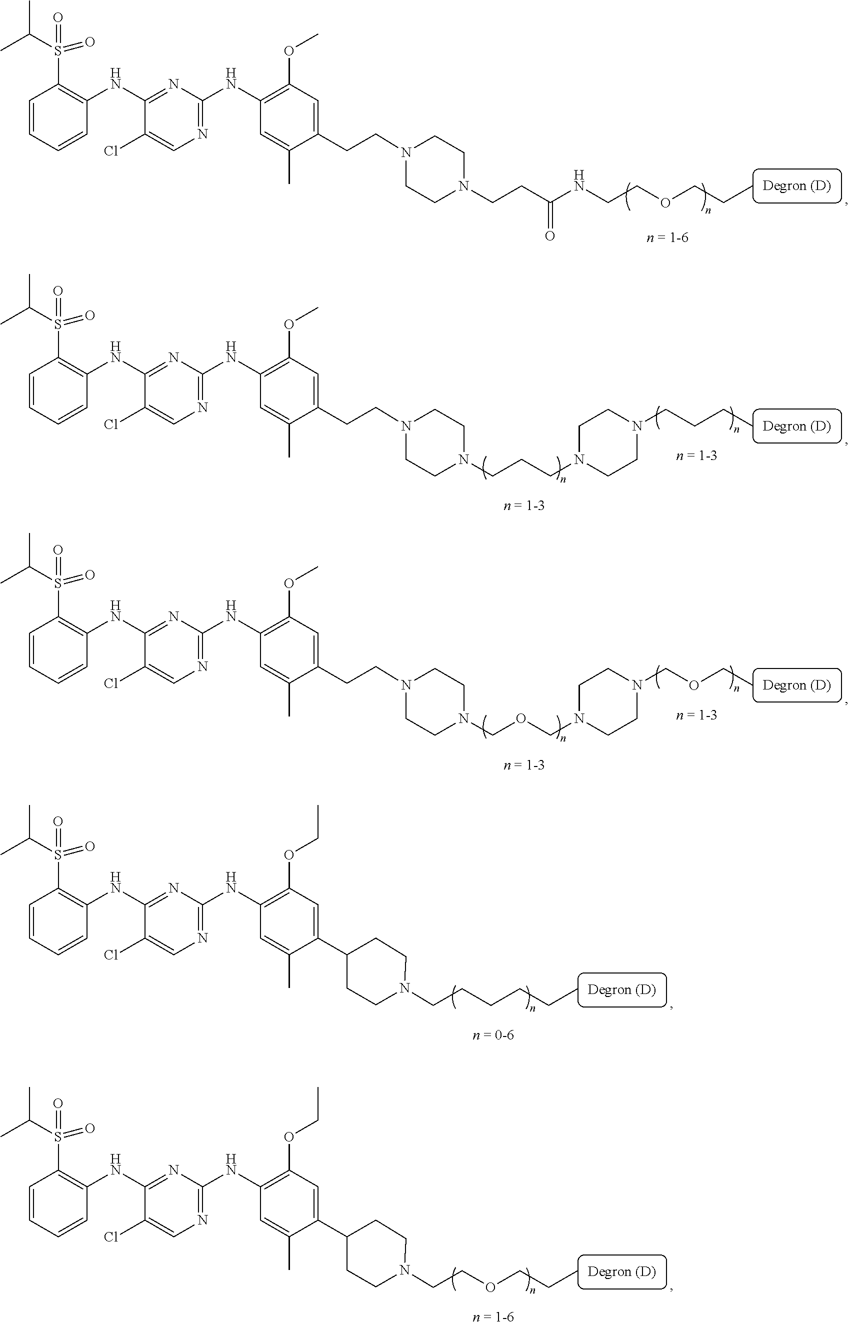 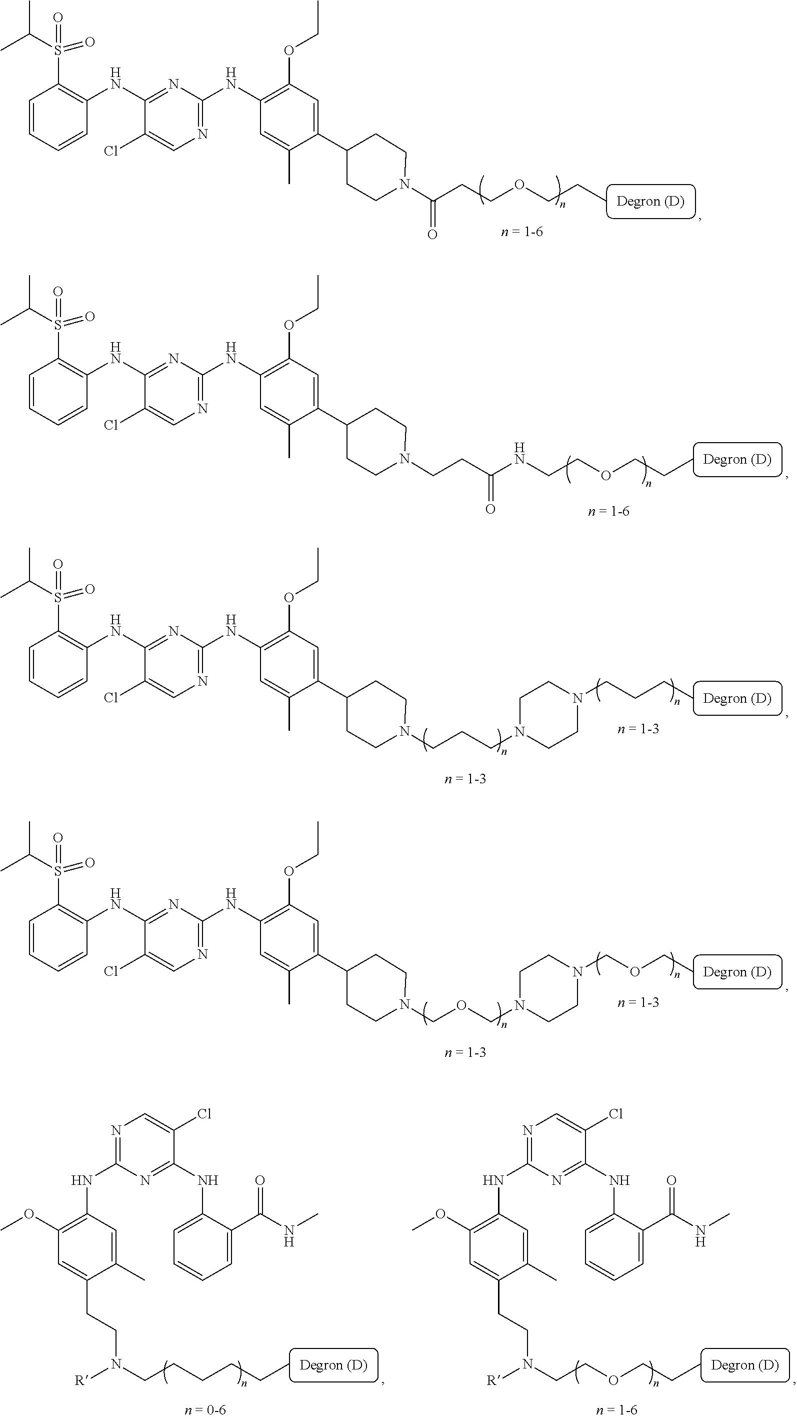 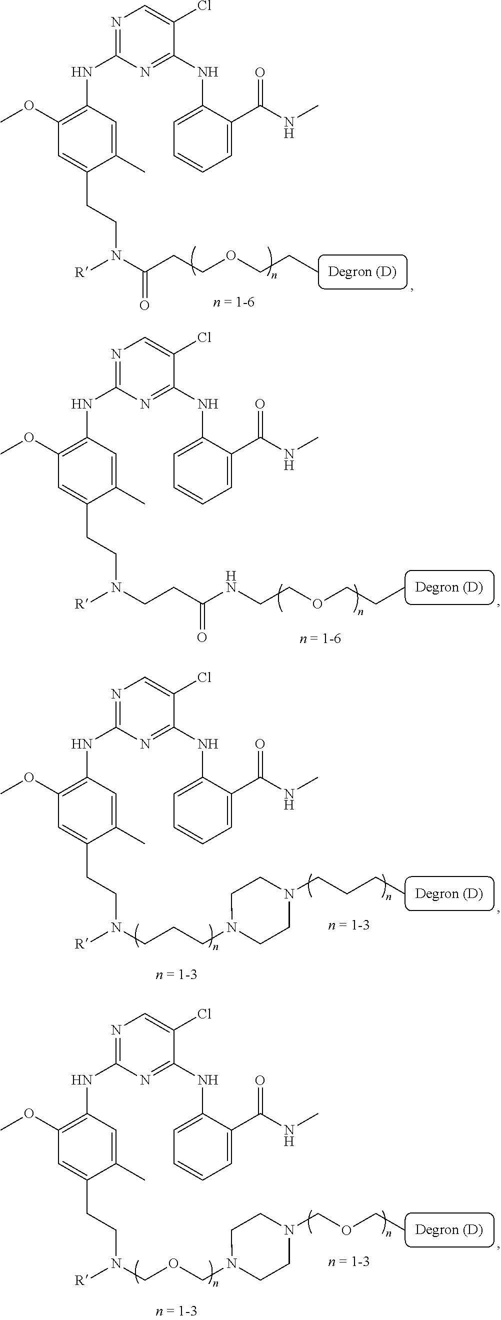 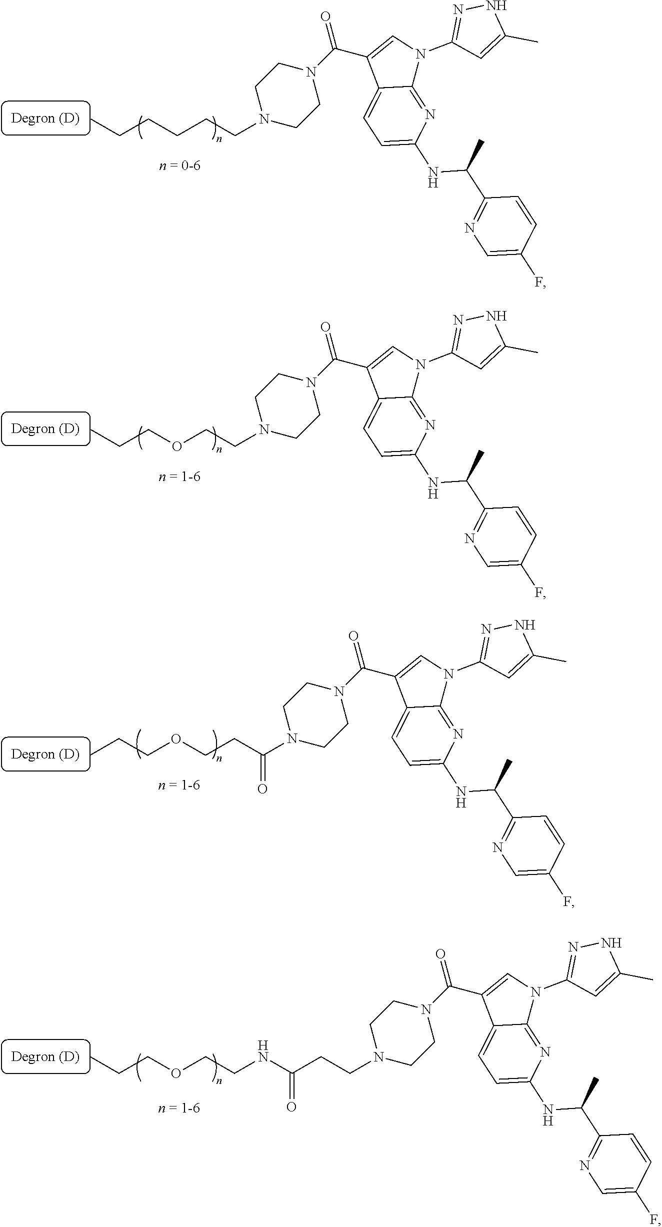 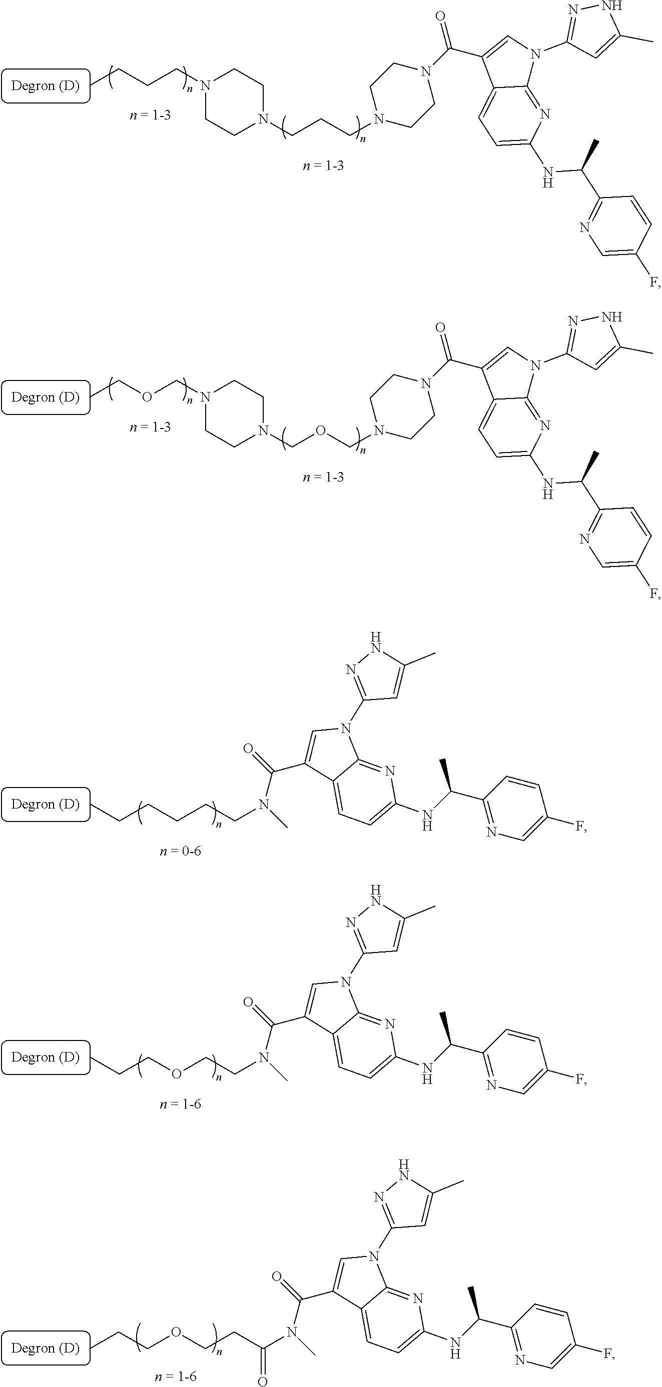 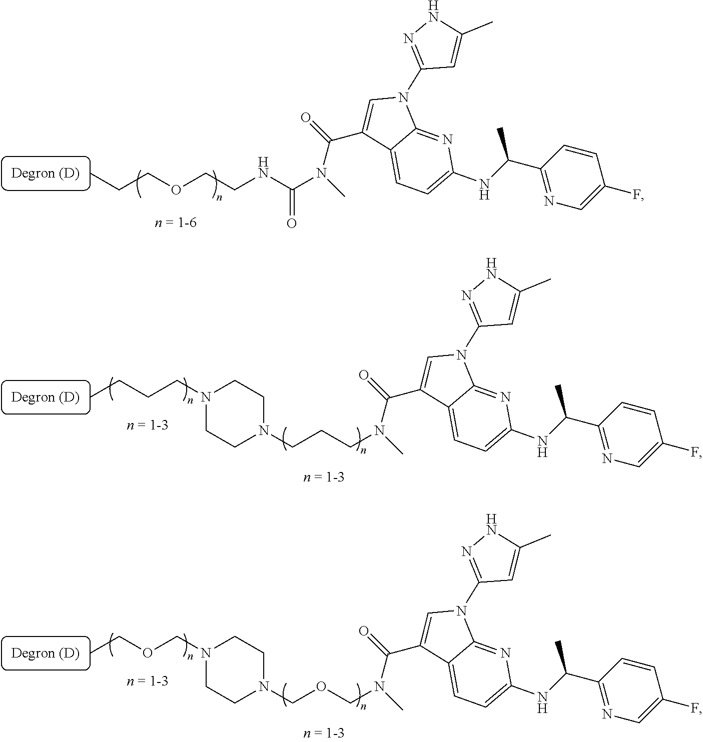 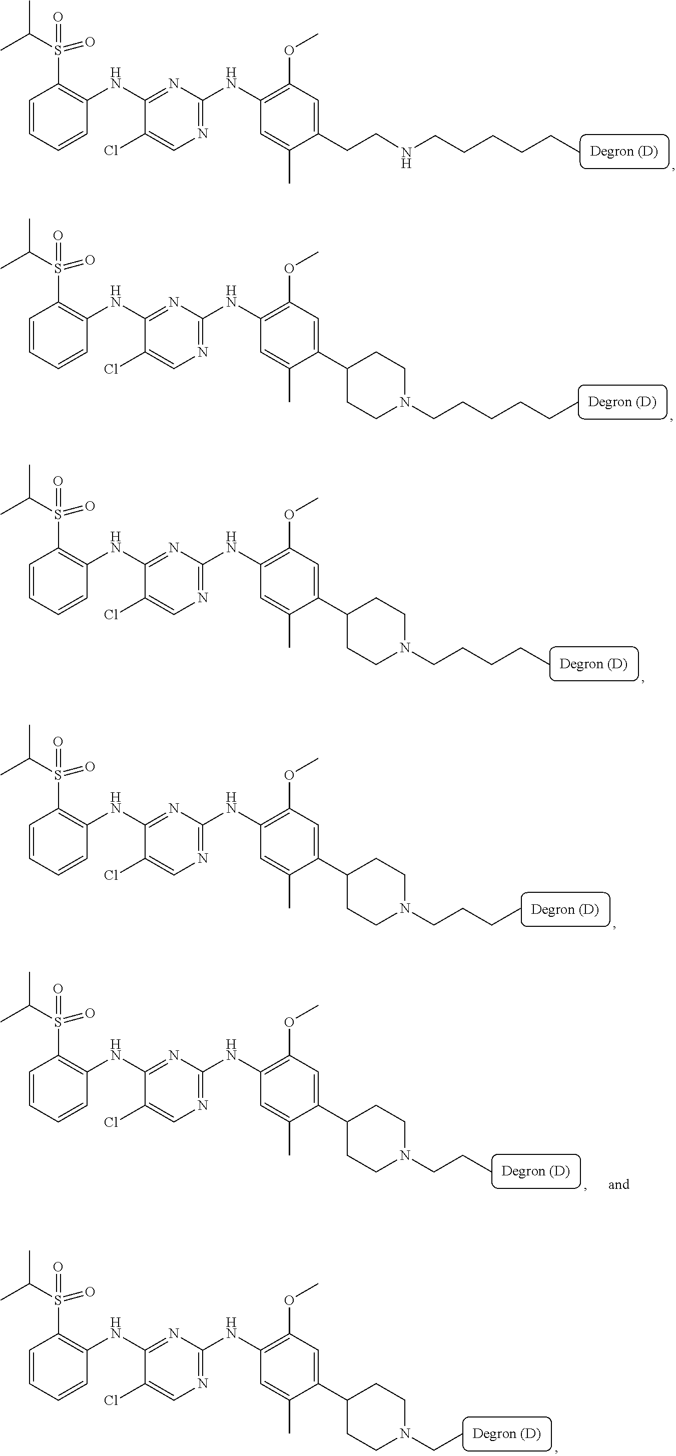
or a pharmaceutically acceptable salt or stereoisomer thereof, wherein R′ is H or Me and wherein each “n” may be the same or different.

30. (canceled)

31. The compound of claim 1, wherein the degron is represented by formula D1: 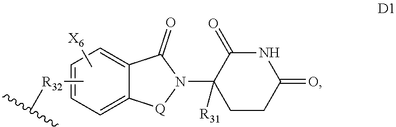
or a stereoisomer thereof
wherein,
Q is CH₂, S, C═O, or 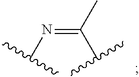 [SMILES: */N=C(\*)C]
R₃₁ is hydrogen or methyl;
R₃₂ is CH₂, NH, O, C≡C, 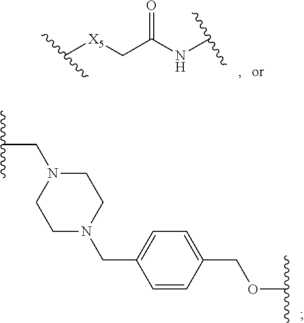
X₅ is absent, CH₂, NH, or O; and
X₆ is alkyl, halo, CN, CF₃, OCHF₂ or OCF₃.

32. The compound of claim 31, wherein the degron is represented by any one of the following structures:

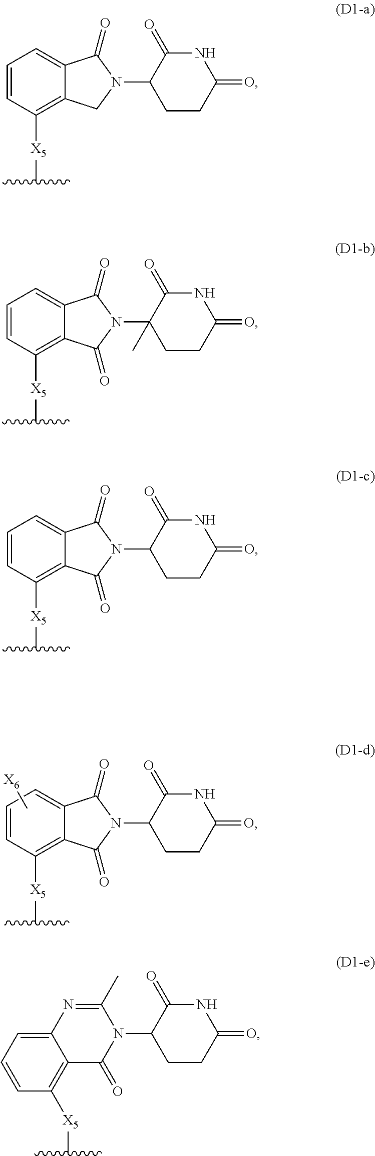

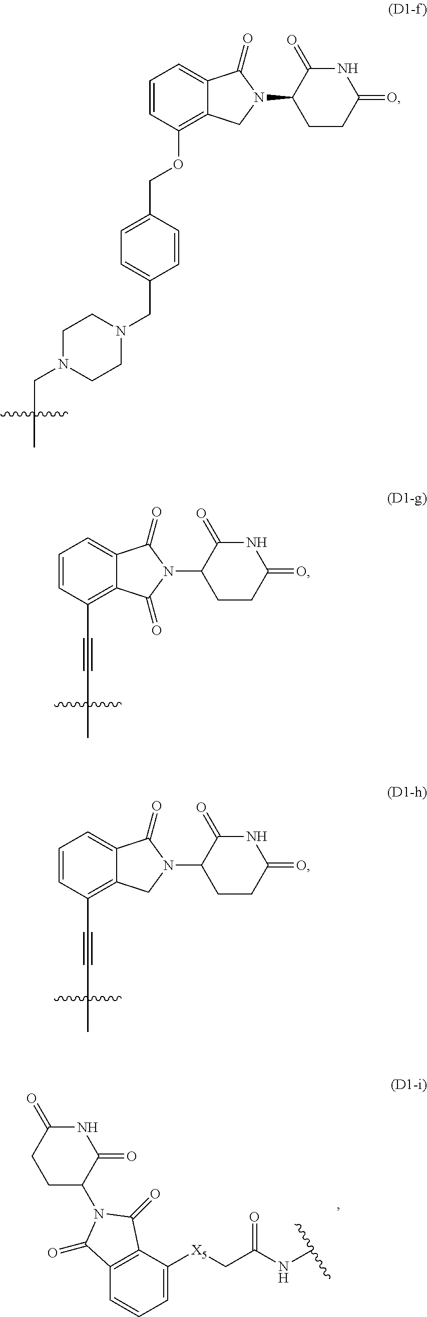

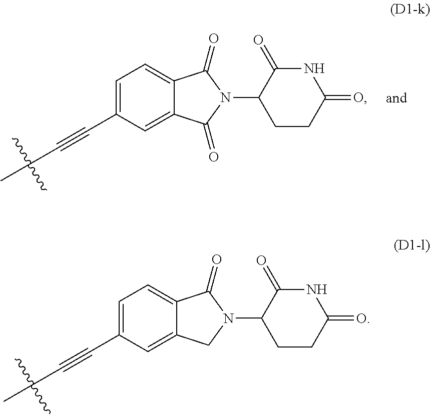 [SMILES: *C#Cc1ccc2c(c1)C(=O)N(C1CCC(=O)NC1=O)C2=O ; *C#Cc1ccc2c(c1)CN(C1CCC(=O)NC1=O)C2=O]

33. (canceled)

34. The compound of claim 1, wherein the degron is represented by any one of the following structures: 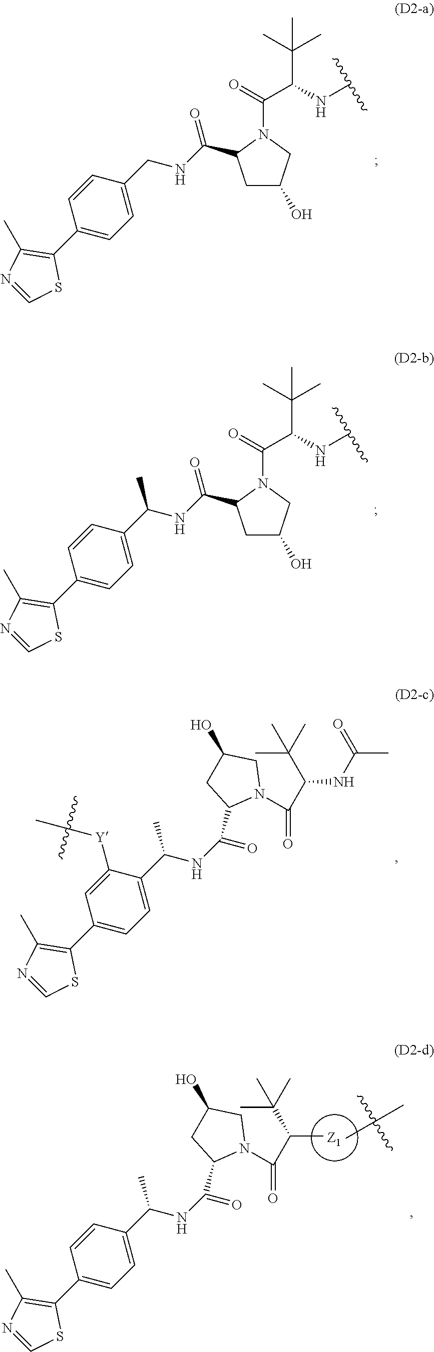
wherein Z₁ is a cyclic group, 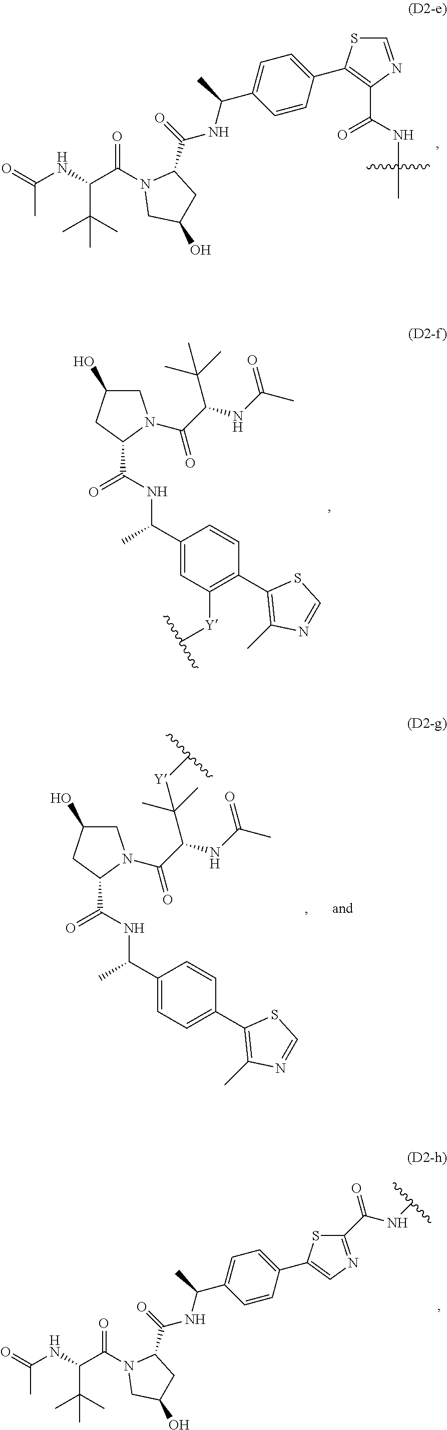
wherein Y′ is a bond, CH₂, NH, NMe, O, or S, or stereoisomer thereof.

35. The compound of claim 34, wherein Z₁ is a 5-6 membered cyclic or 5-6 membered heterocyclic group.

36. The compound of claim 35 wherein Z₁ is

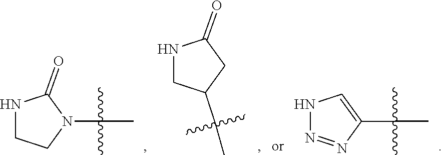 [SMILES: *N1CCNC1=O ; *C1CNC(=O)C1 ; *c1c[nH]nn1]

37. (canceled)

38. The compound of claim 1, wherein the degron is represented by any one of the following structures: 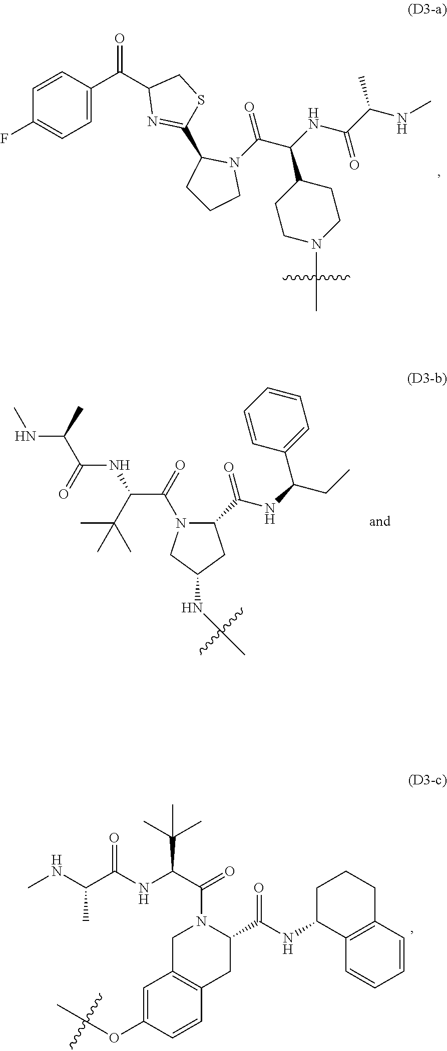
or a stereoisomer thereof.

39. (canceled)

40. The compound of claim 1, wherein the degron is represented by any one of the following structures: 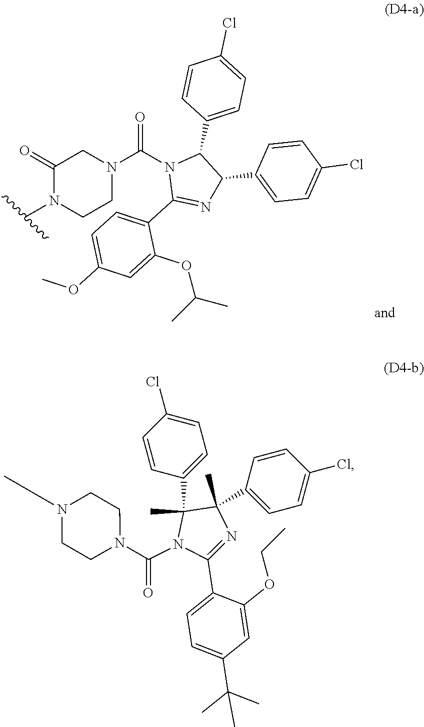 [SMILES: *N1CCN(C(=O)N2C(c3ccc(OC)cc3OC(C)C)=N[C@@H](c3ccc(Cl)cc3)[C@H]2c2ccc(Cl)cc2)CC1=O ; CCOc1cc(C(C)(C)C)ccc1C1=N[C@@](C)(c2ccc(Cl)cc2)[C@@](C)(c2ccc(Cl)cc2)N1C(=O)N1CCN(CC)CC1]
or a stereoisomer thereof.

41. The compound of claim 1, which is represented by any one of the following structures: 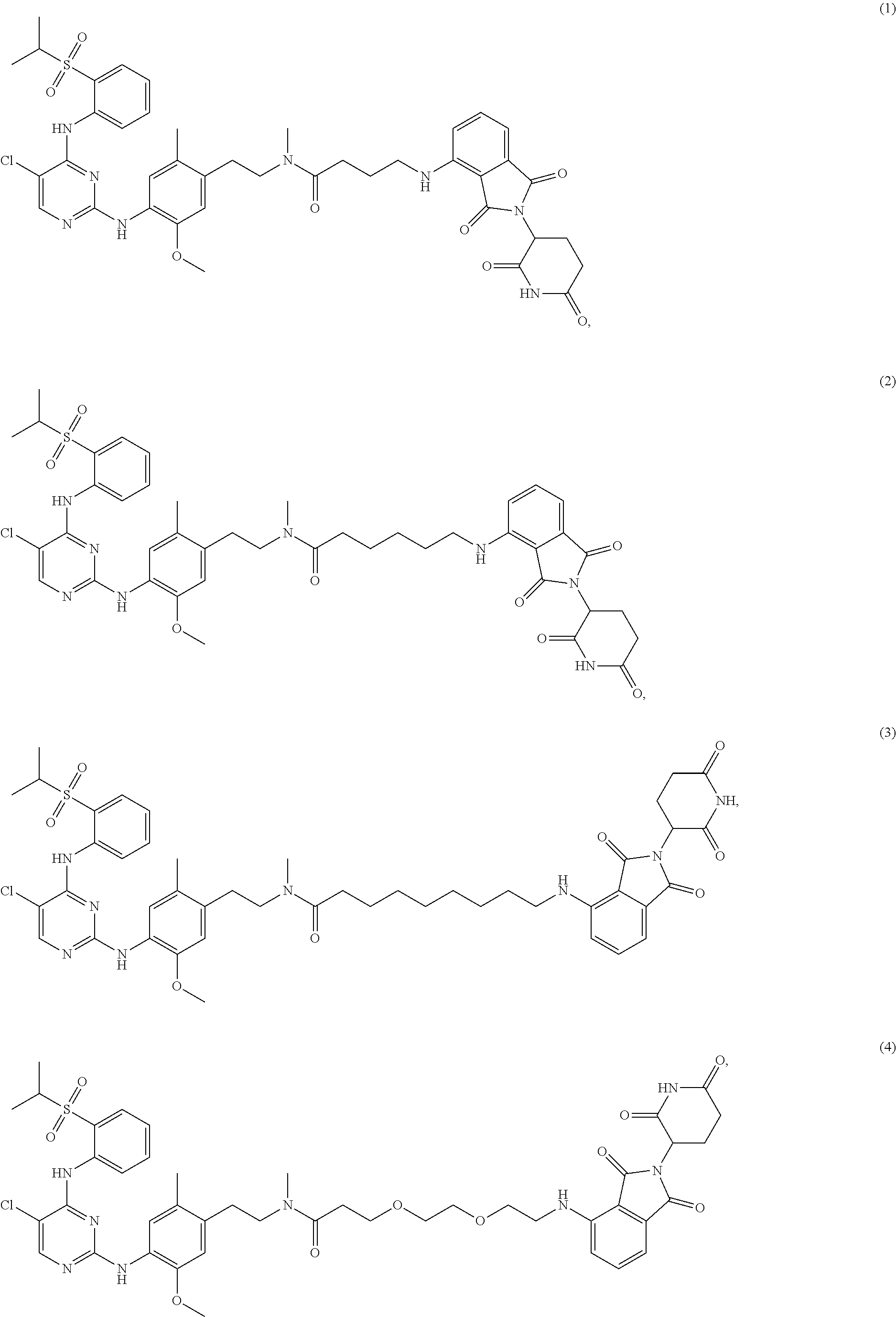 [SMILES: COc1cc(CCN(C)C(=O)CCCNc2cccc3c2C(=O)N(C2CCC(=O)NC2=O)C3=O)c(C)cc1Nc1ncc(Cl)c(Nc2ccccc2S(=O)(=O)C(C)C)n1 ; COc1cc(CCN(C)C(=O)CCCCCNc2cccc3c2C(=O)N(C2CCC(=O)NC2=O)C3=O)c(C)cc1Nc1ncc(Cl)c(Nc2ccccc2S(=O)(=O)C(C)C)n1 ; COc1cc(CCN(C)C(=O)CCCCCCCCNc2cccc3c2C(=O)N(C2CCC(=O)NC2=O)C3=O)c(C)cc1Nc1ncc(Cl)c(Nc2ccccc2S(=O)(=O)C(C)C)n1 ; COc1cc(CCN(C)C(=O)CCOCCOCCNc2cccc3c2C(=O)N(C2CCC(=O)NC2=O)C3=O)c(C)cc1Nc1ncc(Cl)c(Nc2ccccc2S(=O)(=O)C(C)C)n1] 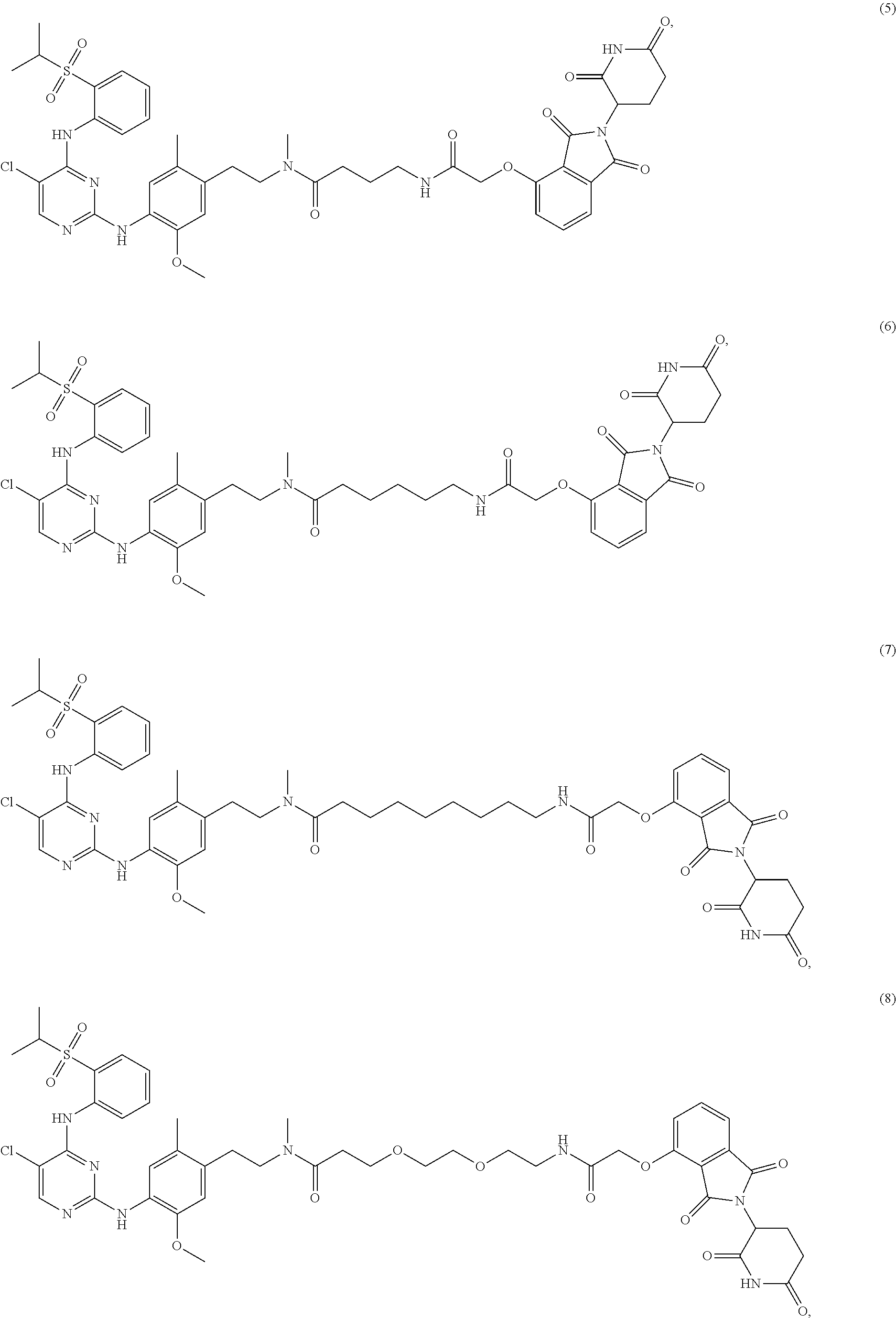 [SMILES: COc1cc(CCN(C)C(=O)CCCNC(=O)COc2cccc3c2C(=O)N(C2CCC(=O)NC2=O)C3=O)c(C)cc1Nc1ncc(Cl)c(Nc2ccccc2S(=O)(=O)C(C)C)n1 ; COc1cc(CCN(C)C(=O)CCCCCNC(=O)COc2cccc3c2C(=O)N(C2CCC(=O)NC2=O)C3=O)c(C)cc1Nc1ncc(Cl)c(Nc2ccccc2S(=O)(=O)C(C)C)n1 ; COc1cc(CCN(C)C(=O)CCCCCCCCNC(=O)COc2cccc3c2C(=O)N(C2CCC(=O)NC2=O)C3=O)c(C)cc1Nc1ncc(Cl)c(Nc2ccccc2S(=O)(=O)C(C)C)n1 ; COc1cc(CCN(C)C(=O)CCOCCOCCNC(=O)COc2cccc3c2C(=O)N(C2CCC(=O)NC2=O)C3=O)c(C)cc1Nc1ncc(Cl)c(Nc2ccccc2S(=O)(=O)C(C)C)n1] 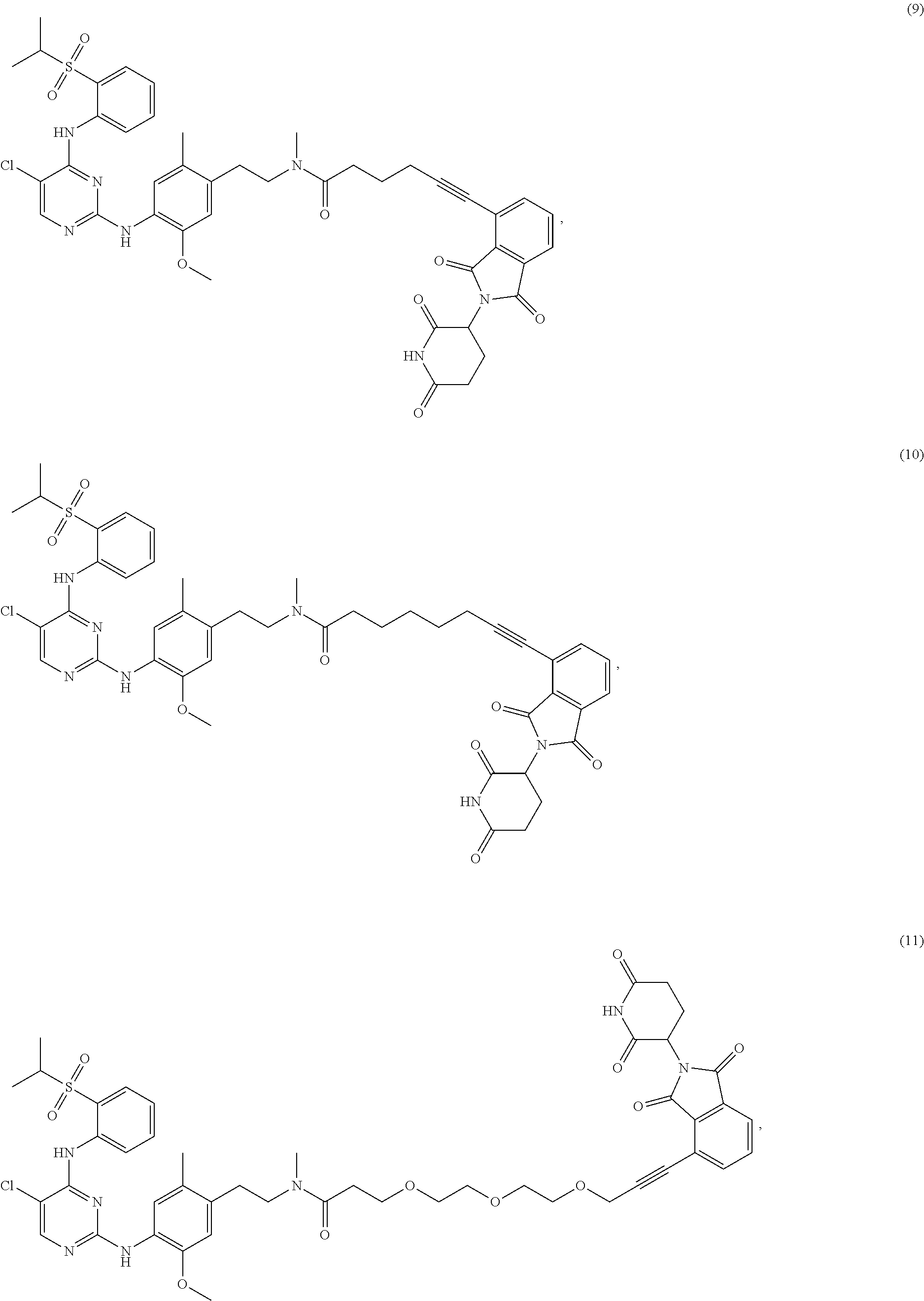 [SMILES: COc1cc(CCN(C)C(=O)CCCC#Cc2cccc3c2C(=O)N(C2CCC(=O)NC2=O)C3=O)c(C)cc1Nc1ncc(Cl)c(Nc2ccccc2S(=O)(=O)C(C)C)n1 ; COc1cc(CCN(C)C(=O)CCCCCC#Cc2cccc3c2C(=O)N(C2CCC(=O)NC2=O)C3=O)c(C)cc1Nc1ncc(Cl)c(Nc2ccccc2S(=O)(=O)C(C)C)n1 ; COc1cc(CCN(C)C(=O)CCOCCOCCOCC#Cc2cccc3c2C(=O)N(C2CCC(=O)NC2=O)C3=O)c(C)cc1Nc1ncc(Cl)c(Nc2ccccc2S(=O)(=O)C(C)C)n1] 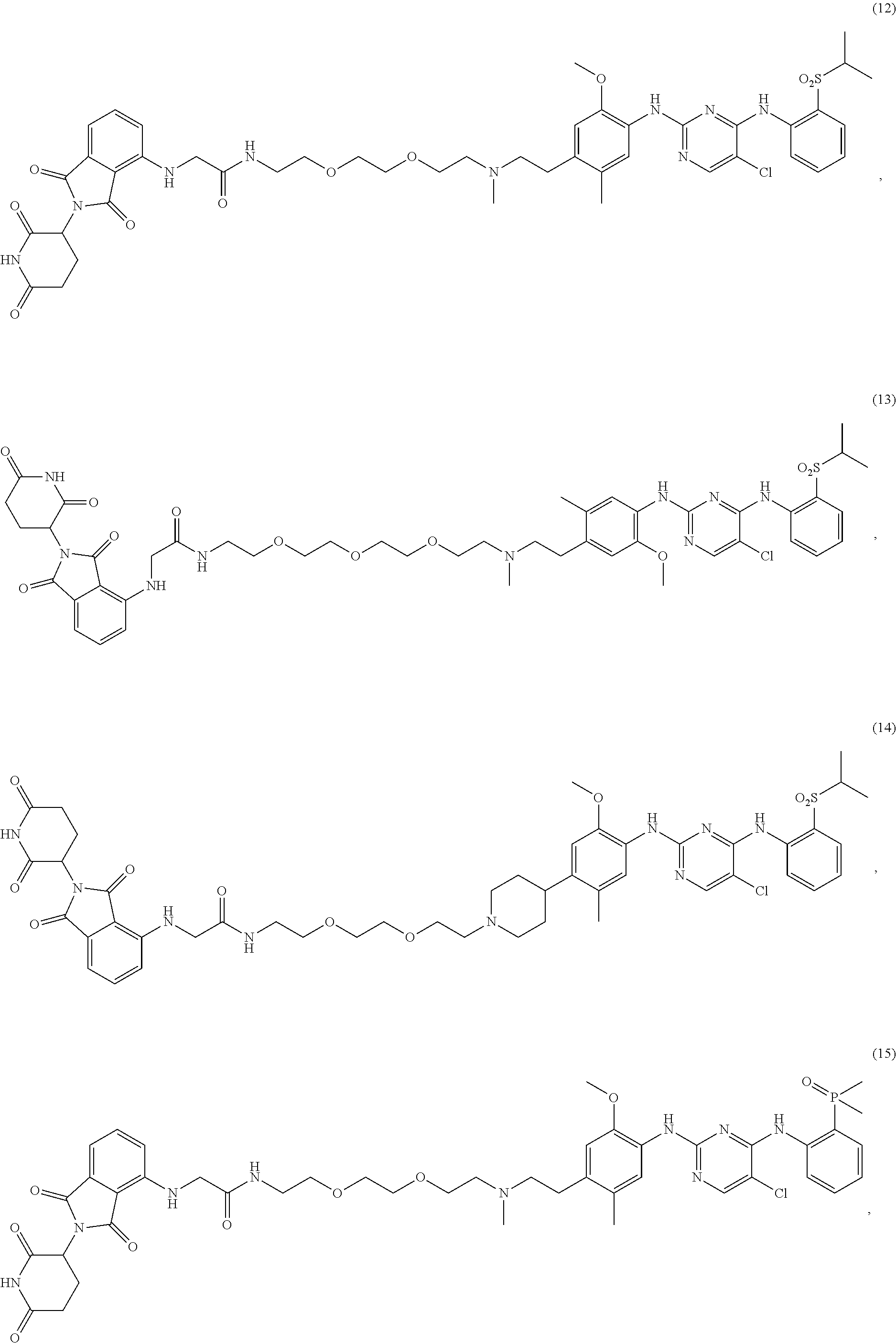 [SMILES: COc1cc(CCN(C)CCOCCOCCNC(=O)CNc2cccc3c2C(=O)N(C2CCC(=O)NC2=O)C3=O)c(C)cc1Nc1ncc(Cl)c(Nc2ccccc2S(=O)(=O)C(C)C)n1 ; COc1cc(CCN(C)CCOCCOCCOCCNC(=O)CNc2cccc3c2C(=O)N(C2CCC(=O)NC2=O)C3=O)c(C)cc1Nc1ncc(Cl)c(Nc2ccccc2S(=O)(=O)C(C)C)n1 ; COc1cc(C2CCN(CCOCCOCCNC(=O)CNc3cccc4c3C(=O)N(C3CCC(=O)NC3=O)C4=O)CC2)c(C)cc1Nc1ncc(Cl)c(Nc2ccccc2S(=O)(=O)C(C)C)n1 ; COc1cc(CCN(C)CCOCCOCCNC(=O)CNc2cccc3c2C(=O)N(C2CCC(=O)NC2=O)C3=O)c(C)cc1Nc1ncc(Cl)c(Nc2ccccc2P(C)(C)=O)n1] 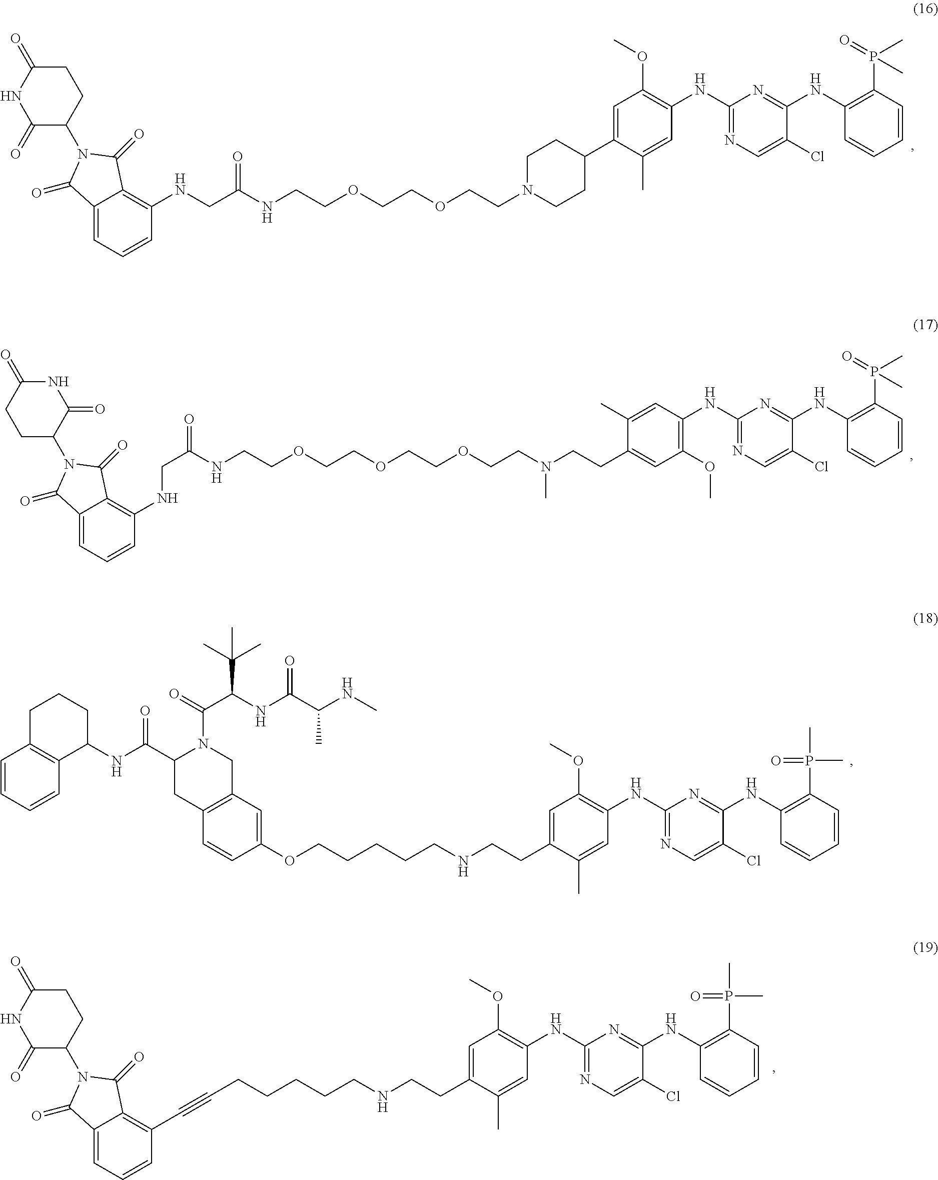 [SMILES: COc1cc(C2CCN(CCOCCOCCNC(=O)CNc3cccc4c3C(=O)N(C3CCC(=O)NC3=O)C4=O)CC2)c(C)cc1Nc1ncc(Cl)c(Nc2ccccc2P(C)(C)=O)n1 ; COc1cc(CCN(C)CCOCCOCCOCCNC(=O)CNc2cccc3c2C(=O)N(C2CCC(=O)NC2=O)C3=O)c(C)cc1Nc1ncc(Cl)c(Nc2ccccc2P(C)(C)=O)n1 ; CN[C@H](C)C(=O)N[C@@H](C(=O)N1Cc2cc(OCCCCCNCCc3cc(OC)c(Nc4ncc(Cl)c(Nc5ccccc5P(C)(C)=O)n4)cc3C)ccc2CC1C(=O)NC1CCCc2ccccc21)C(C)(C)C ; COc1cc(CCNCCCCCC#Cc2cccc3c2C(=O)N(C2CCC(=O)NC2=O)C3=O)c(C)cc1Nc1ncc(Cl)c(Nc2ccccc2P(C)(C)=O)n1] 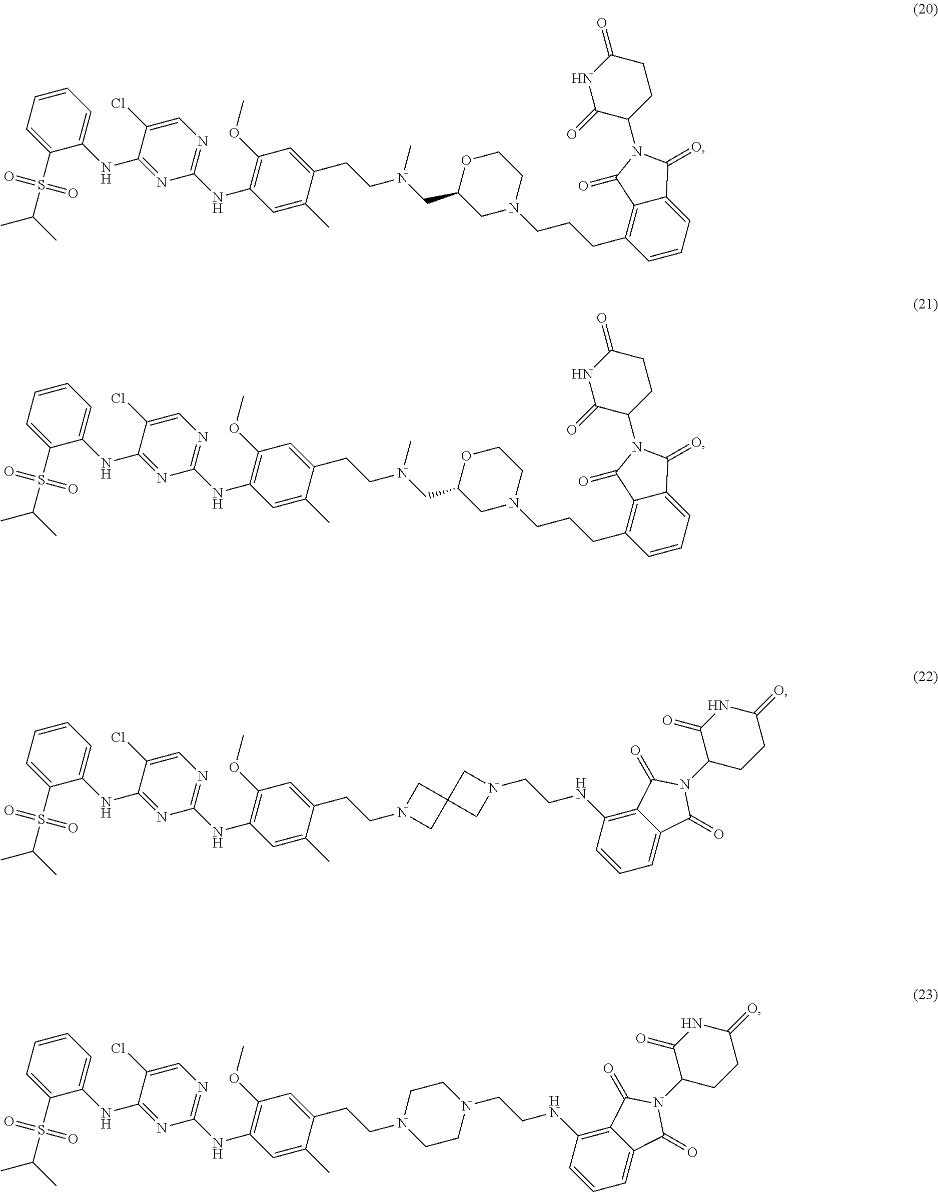 [SMILES: COc1cc(CCN(C)C[C@@H]2CN(CCCc3cccc4c3C(=O)N(C3CCC(=O)NC3=O)C4=O)CCO2)c(C)cc1Nc1ncc(Cl)c(Nc2ccccc2S(=O)(=O)C(C)C)n1 ; COc1cc(CCN(C)C[C@H]2CN(CCCc3cccc4c3C(=O)N(C3CCC(=O)NC3=O)C4=O)CCO2)c(C)cc1Nc1ncc(Cl)c(Nc2ccccc2S(=O)(=O)C(C)C)n1 ; COc1cc(CCN2CC3(CN(CCNc4cccc5c4C(=O)N(C4CCC(=O)NC4=O)C5=O)C3)C2)c(C)cc1Nc1ncc(Cl)c(Nc2ccccc2S(=O)(=O)C(C)C)n1 ; COc1cc(CCN2CCN(CCNc3cccc4c3C(=O)N(C3CCC(=O)NC3=O)C4=O)CC2)c(C)cc1Nc1ncc(Cl)c(Nc2ccccc2S(=O)(=O)C(C)C)n1] 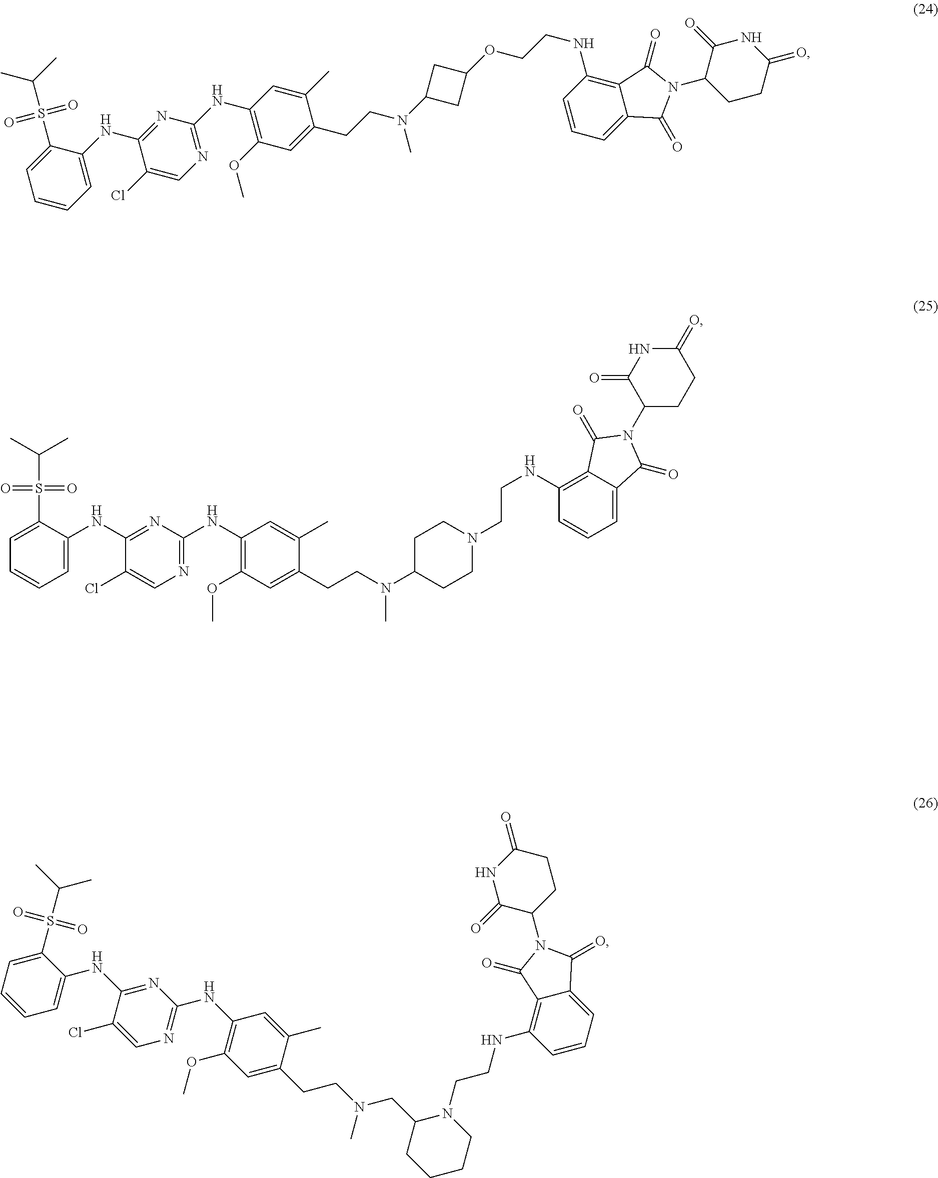 [SMILES: COc1cc(CCN(C)C2CC(OCCNc3cccc4c3C(=O)N(C3CCC(=O)NC3=O)C4=O)C2)c(C)cc1Nc1ncc(Cl)c(Nc2ccccc2S(=O)(=O)C(C)C)n1 ; COc1cc(CCN(C)C2CCN(CCNc3cccc4c3C(=O)N(C3CCC(=O)NC3=O)C4=O)CC2)c(C)cc1Nc1ncc(Cl)c(Nc2ccccc2S(=O)(=O)C(C)C)n1 ; COc1cc(CCN(C)CC2CCCCN2CCNc2cccc3c2C(=O)N(C2CCC(=O)NC2=O)C3=O)c(C)cc1Nc1ncc(Cl)c(Nc2ccccc2S(=O)(=O)C(C)C)n1] 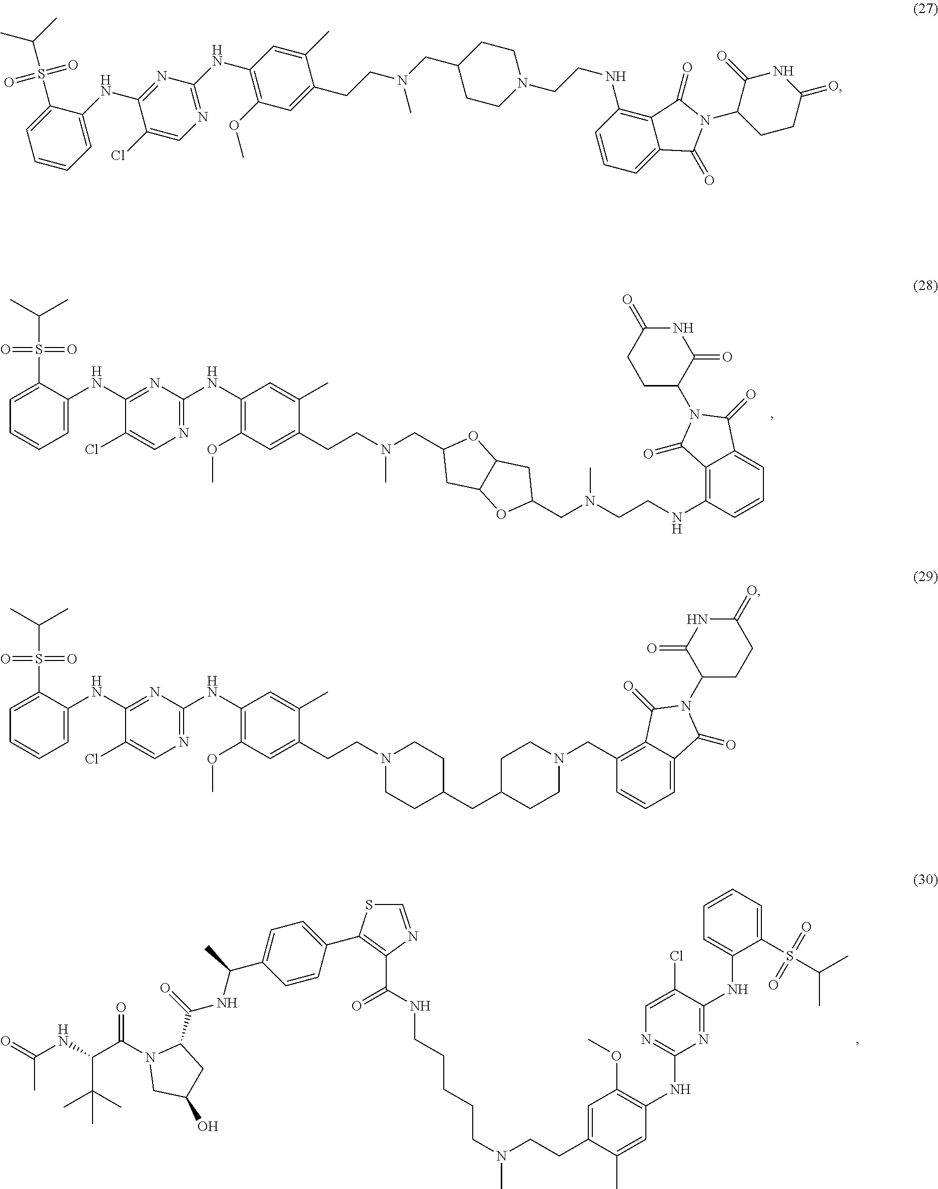 [SMILES: COc1cc(CCN(C)CC2CCN(CCNc3cccc4c3C(=O)N(C3CCC(=O)NC3=O)C4=O)CC2)c(C)cc1Nc1ncc(Cl)c(Nc2ccccc2S(=O)(=O)C(C)C)n1 ; COc1cc(CCN(C)CC2CC3OC(CN(C)CCNc4cccc5c4C(=O)N(C4CCC(=O)NC4=O)C5=O)CC3O2)c(C)cc1Nc1ncc(Cl)c(Nc2ccccc2S(=O)(=O)C(C)C)n1 ; COc1cc(CCN2CCC(CC3CCN(Cc4cccc5c4C(=O)N(C4CCC(=O)NC4=O)C5=O)CC3)CC2)c(C)cc1Nc1ncc(Cl)c(Nc2ccccc2S(=O)(=O)C(C)C)n1 ; COc1cc(CCN(C)CCCCCNC(=O)c2ncsc2-c2ccc([C@H](C)NC(=O)[C@@H]3C[C@@H](O)CN3C(=O)[C@@H](NC(C)=O)C(C)(C)C)cc2)c(C)cc1Nc1ncc(Cl)c(Nc2ccccc2S(=O)(=O)C(C)C)n1] 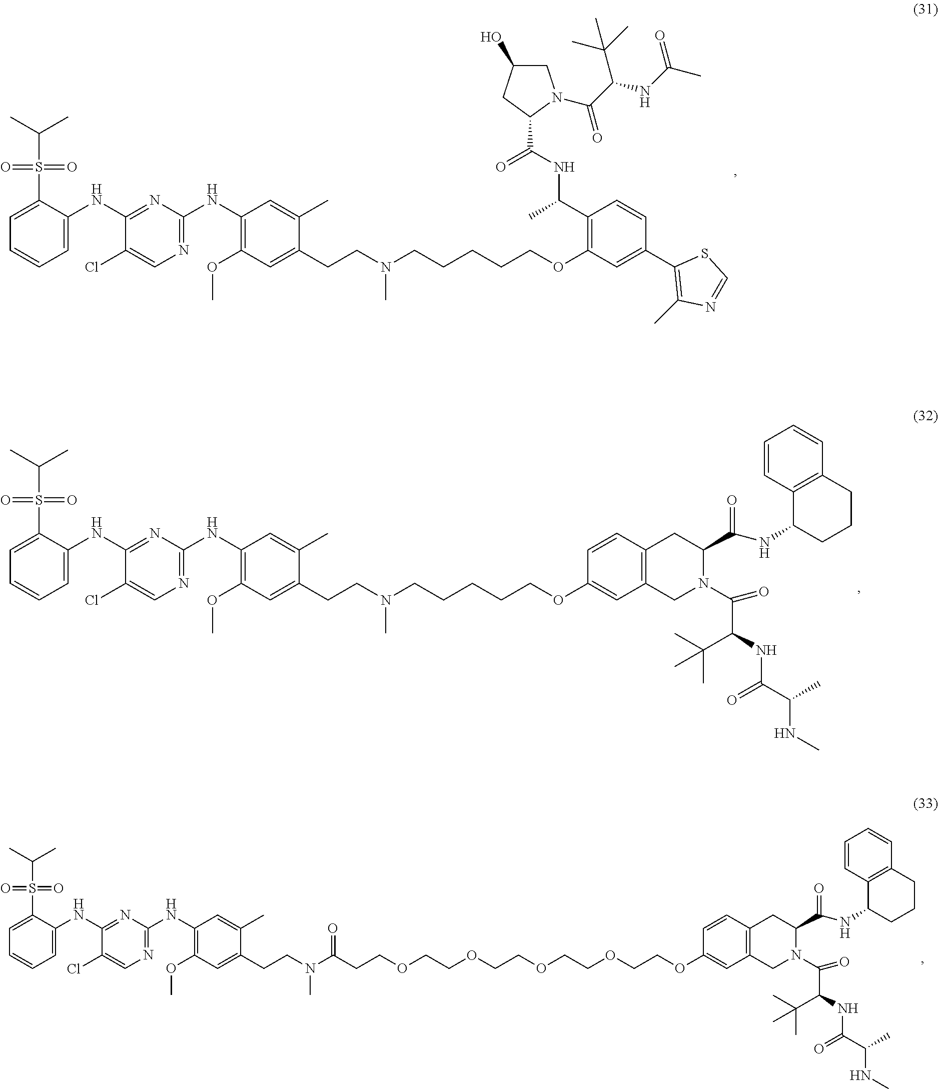 [SMILES: COc1cc(CCN(C)CCCCCOc2cc(-c3scnc3C)ccc2[C@H](C)NC(=O)[C@@H]2C[C@@H](O)CN2C(=O)[C@@H](NC(C)=O)C(C)(C)C)c(C)cc1Nc1ncc(Cl)c(Nc2ccccc2S(=O)(=O)C(C)C)n1 ; CN[C@@H](C)C(=O)N[C@H](C(=O)N1Cc2cc(OCCCCCN(C)CCc3cc(OC)c(Nc4ncc(Cl)c(Nc5ccccc5S(=O)(=O)C(C)C)n4)cc3C)ccc2C[C@H]1C(=O)N[C@H]1CCCc2ccccc21)C(C)(C)C ; CN[C@@H](C)C(=O)N[C@H](C(=O)N1Cc2cc(OCCOCCOCCOCCOCCC(=O)N(C)CCc3cc(OC)c(Nc4ncc(Cl)c(Nc5ccccc5S(=O)(=O)C(C)C)n4)cc3C)ccc2C[C@H]1C(=O)N[C@H]1CCCc2ccccc21)C(C)(C)C] 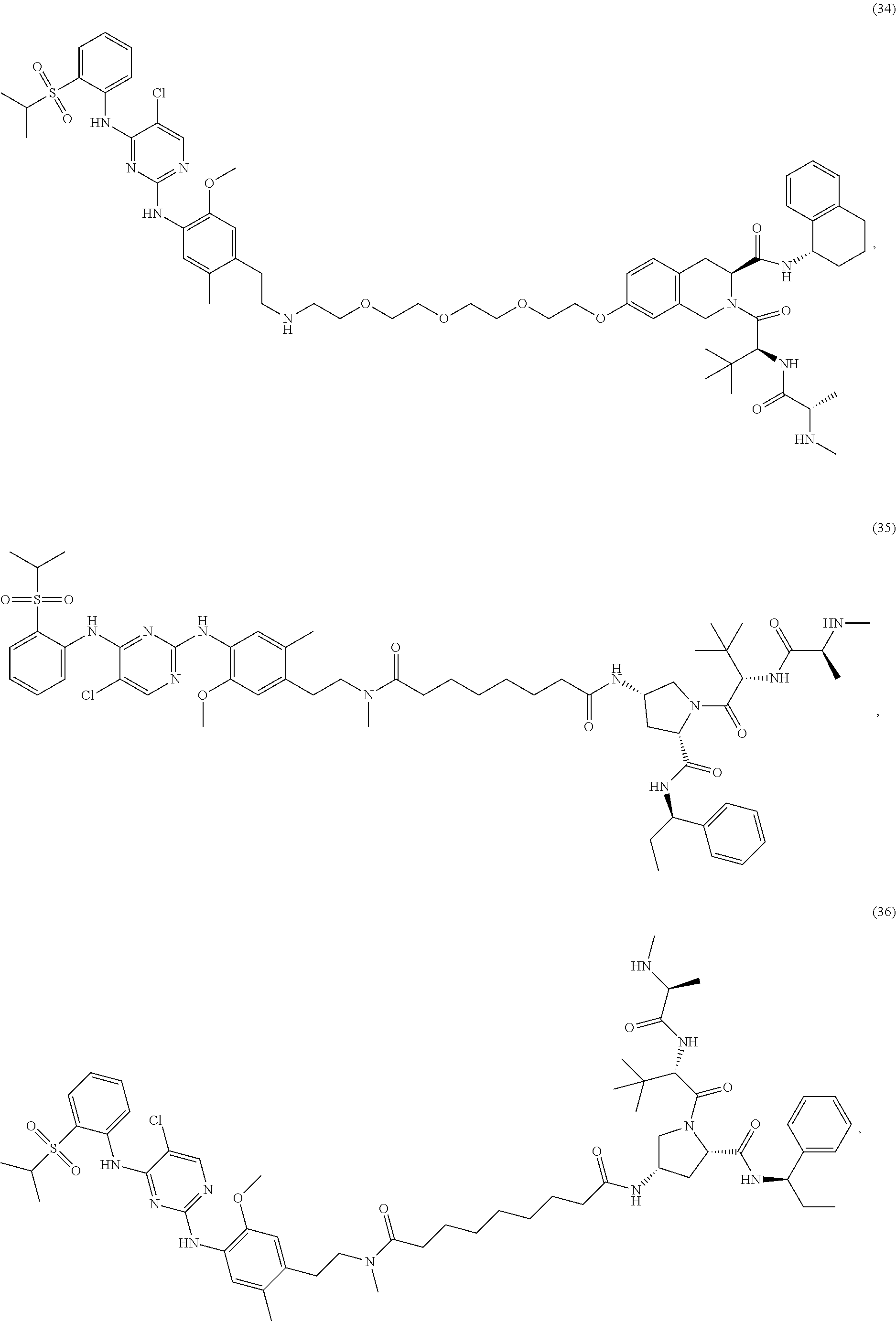 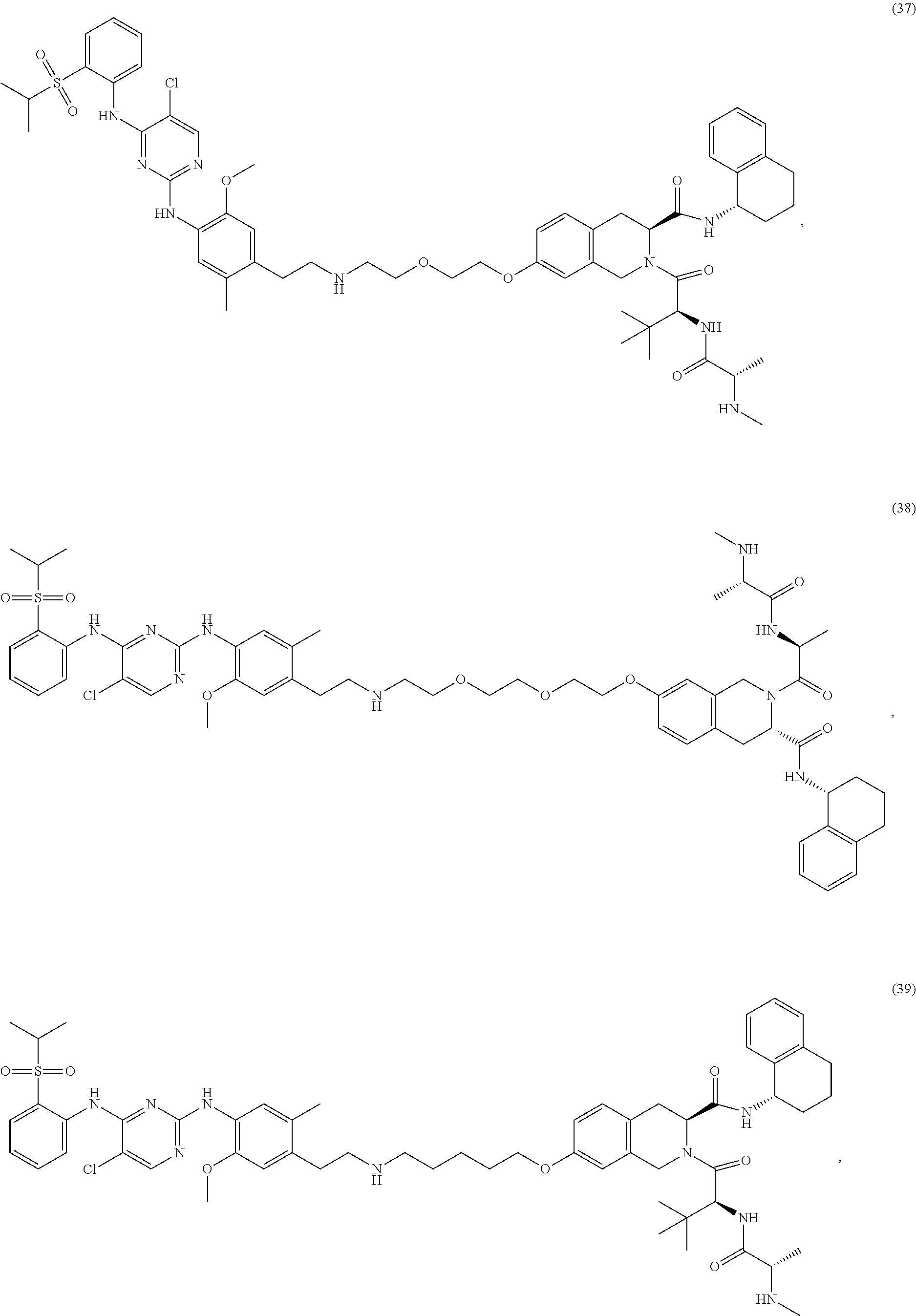 [SMILES: CN[C@@H](C)C(=O)N[C@H](C(=O)N1Cc2cc(OCCOCCNCCc3cc(OC)c(Nc4ncc(Cl)c(Nc5ccccc5S(=O)(=O)C(C)C)n4)cc3C)ccc2C[C@H]1C(=O)N[C@H]1CCCc2ccccc21)C(C)(C)C ; CN[C@@H](C)C(=O)N[C@@H](C)C(=O)N1Cc2cc(OCCOCCOCCNCCc3cc(OC)c(Nc4ncc(Cl)c(Nc5ccccc5S(=O)(=O)C(C)C)n4)cc3C)ccc2C[C@H]1C(=O)N[C@@H]1CCCc2ccccc21 ; CN[C@@H](C)C(=O)N[C@H](C(=O)N1Cc2cc(OCCCCCNCCc3cc(OC)c(Nc4ncc(Cl)c(Nc5ccccc5S(=O)(=O)C(C)C)n4)cc3C)ccc2C[C@H]1C(=O)N[C@H]1CCCc2ccccc21)C(C)(C)C] 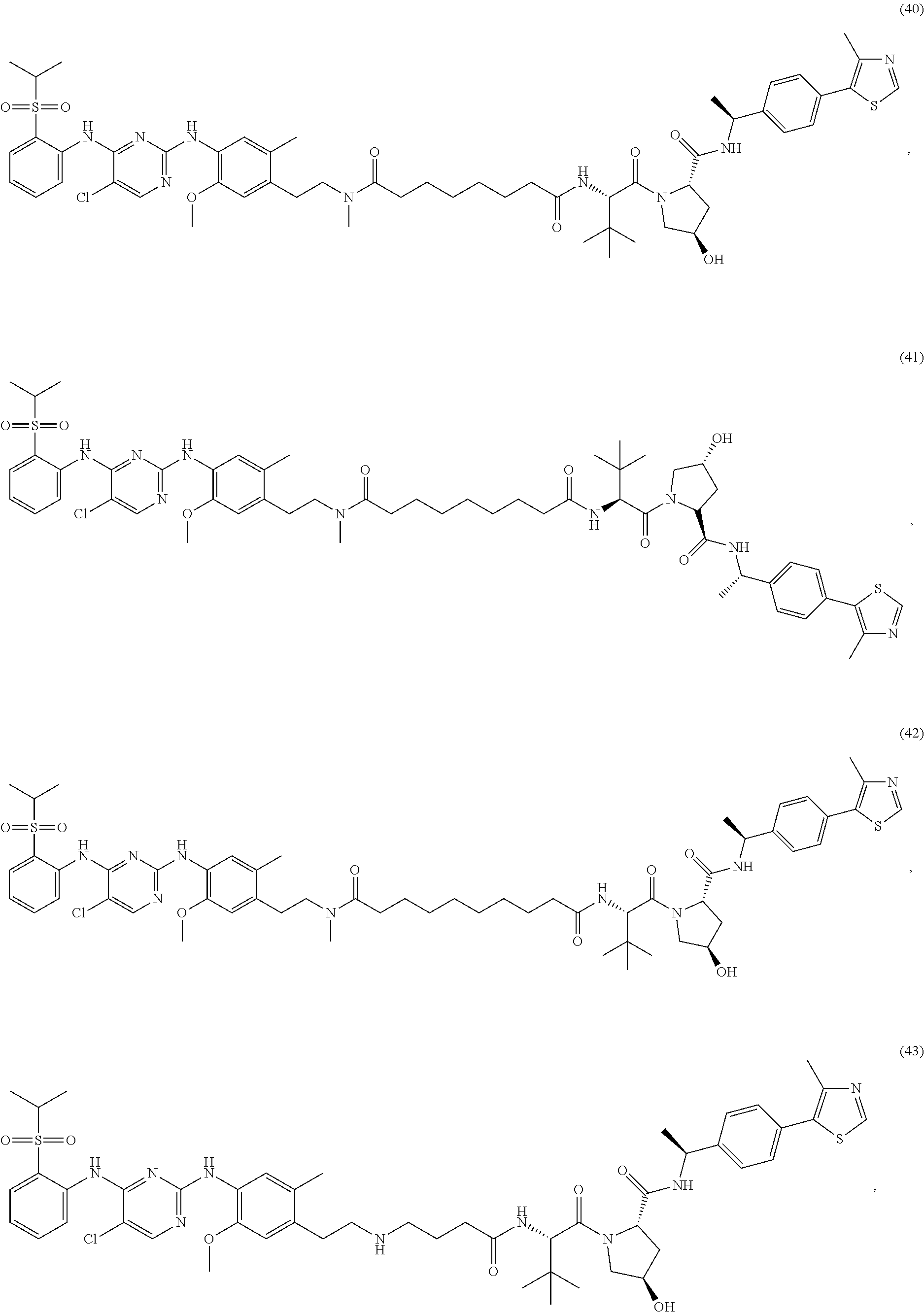 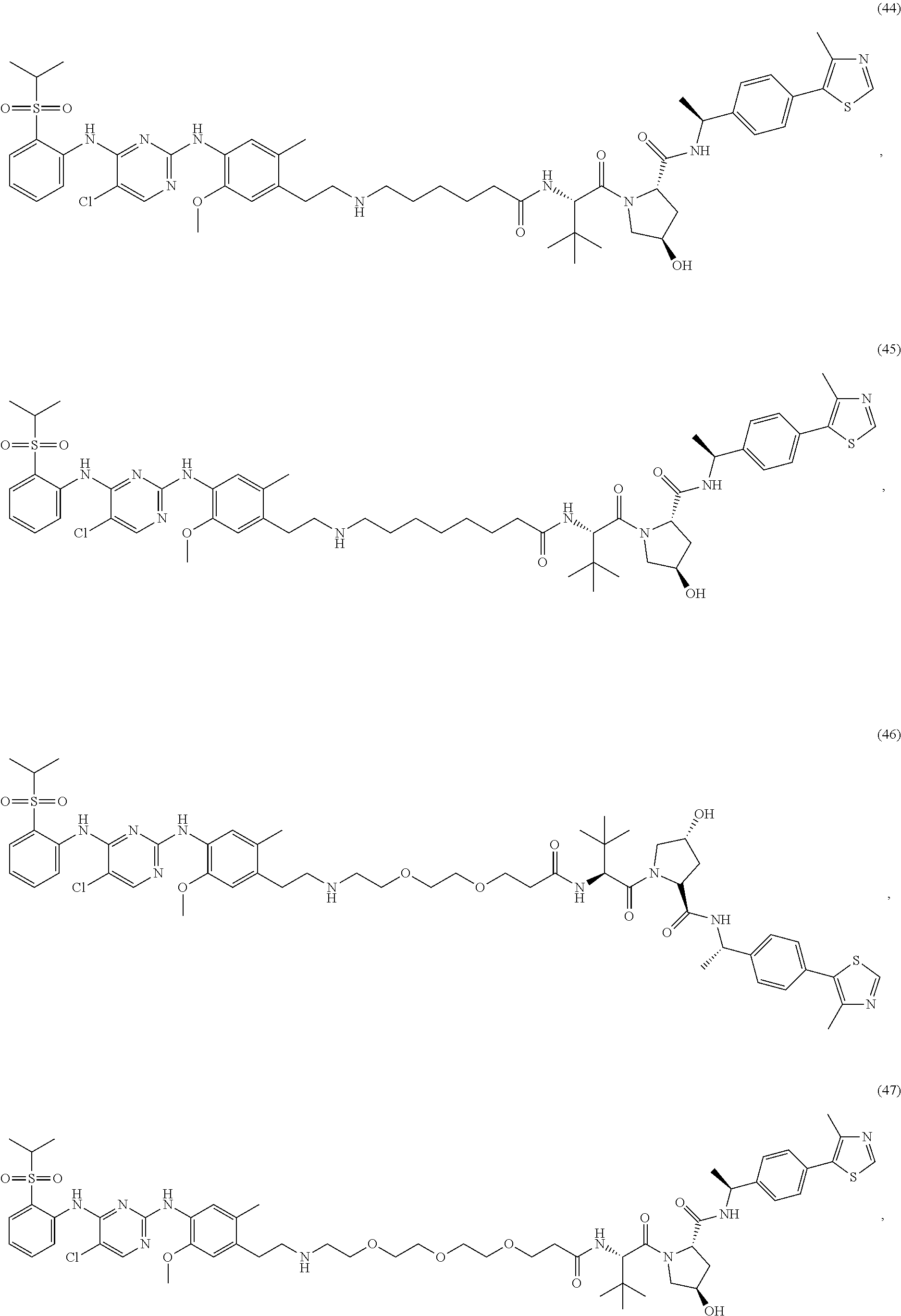 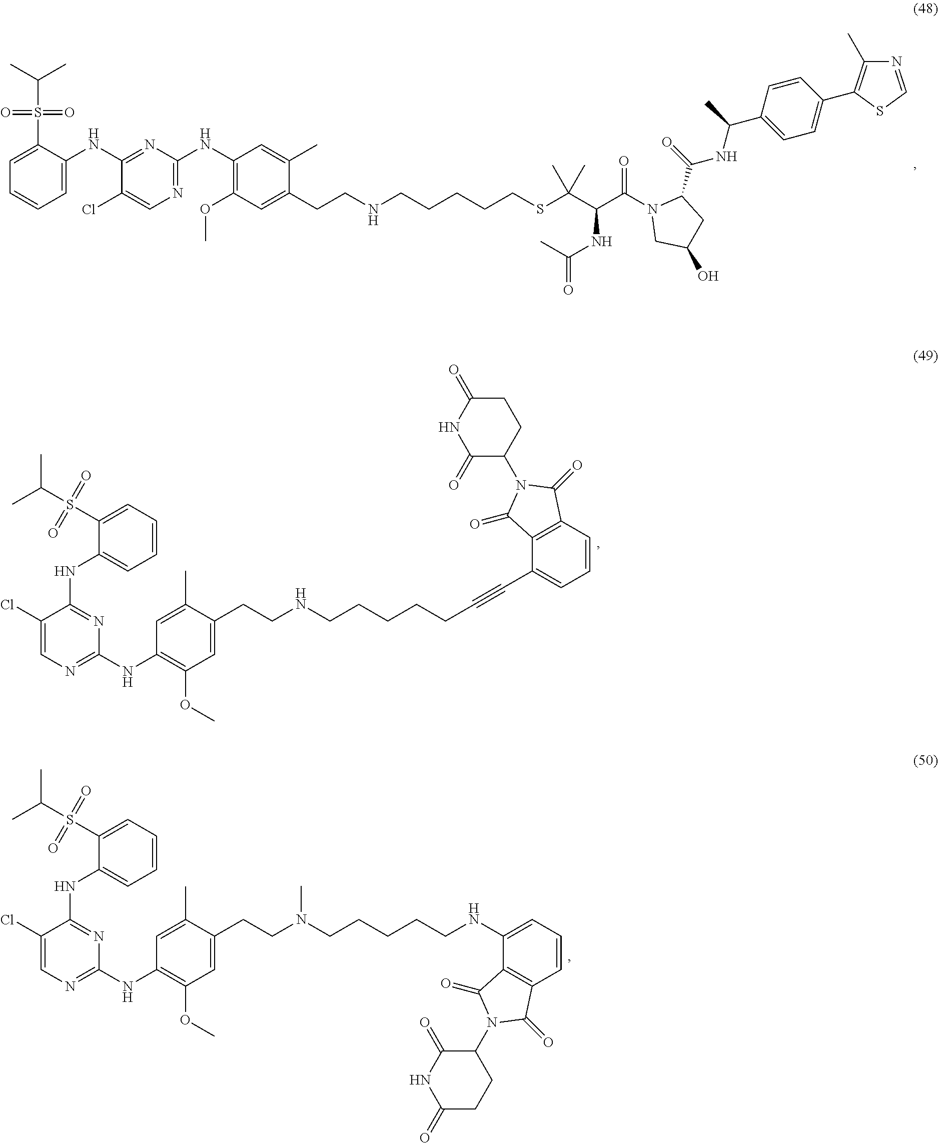 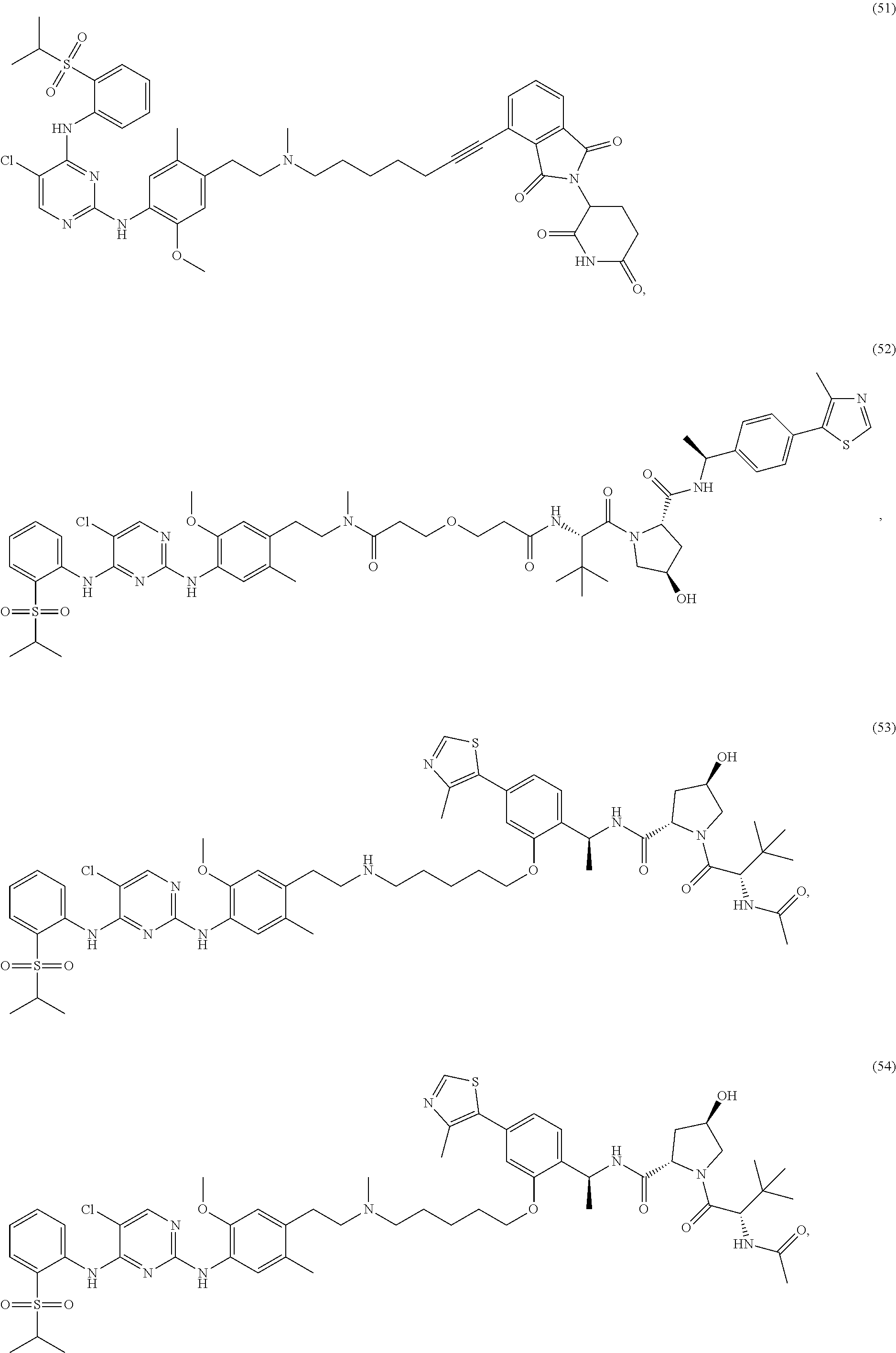 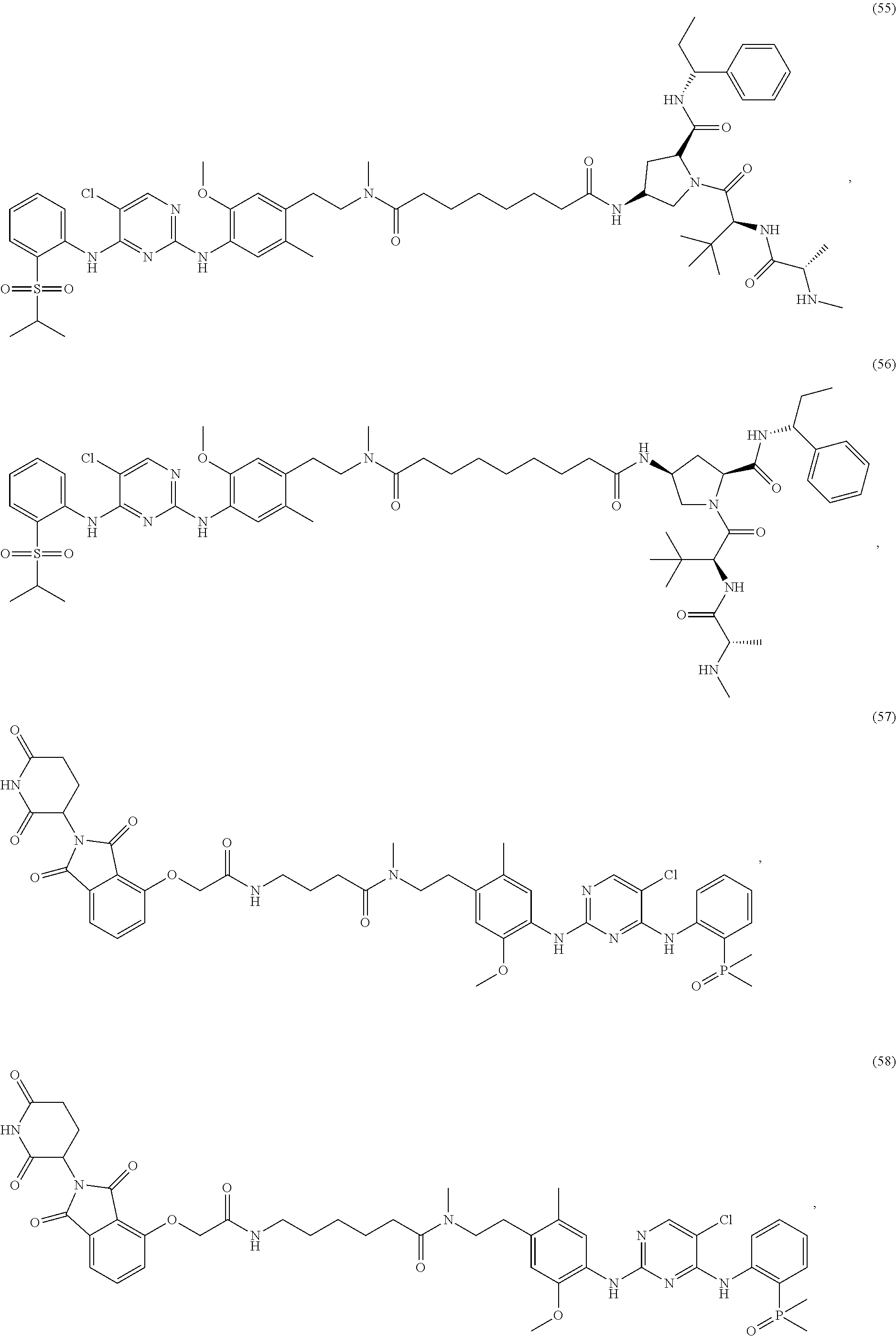 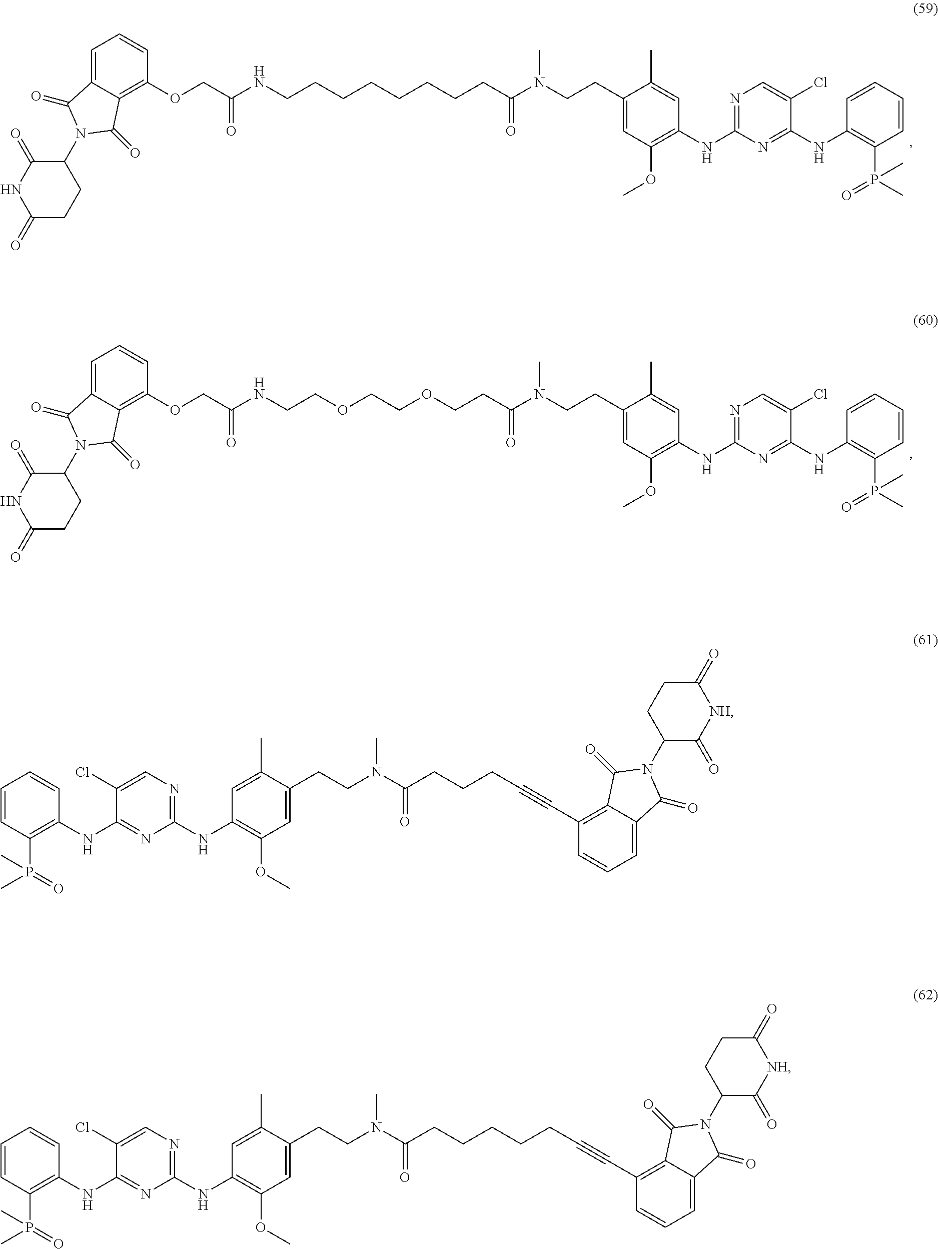 [SMILES: COc1cc(CCN(C)C(=O)CCCCCCCCNC(=O)COc2cccc3c2C(=O)N(C2CCC(=O)NC2=O)C3=O)c(C)cc1Nc1ncc(Cl)c(Nc2ccccc2P(C)(C)=O)n1 ; COc1cc(CCN(C)C(=O)CCOCCOCCNC(=O)COc2cccc3c2C(=O)N(C2CCC(=O)NC2=O)C3=O)c(C)cc1Nc1ncc(Cl)c(Nc2ccccc2P(C)(C)=O)n1 ; COc1cc(CCN(C)C(=O)CCCC#Cc2cccc3c2C(=O)N(C2CCC(=O)NC2=O)C3=O)c(C)cc1Nc1ncc(Cl)c(Nc2ccccc2P(C)(C)=O)n1 ; COc1cc(CCN(C)C(=O)CCCCCC#Cc2cccc3c2C(=O)N(C2CCC(=O)NC2=O)C3=O)c(C)cc1Nc1ncc(Cl)c(Nc2ccccc2P(C)(C)=O)n1] 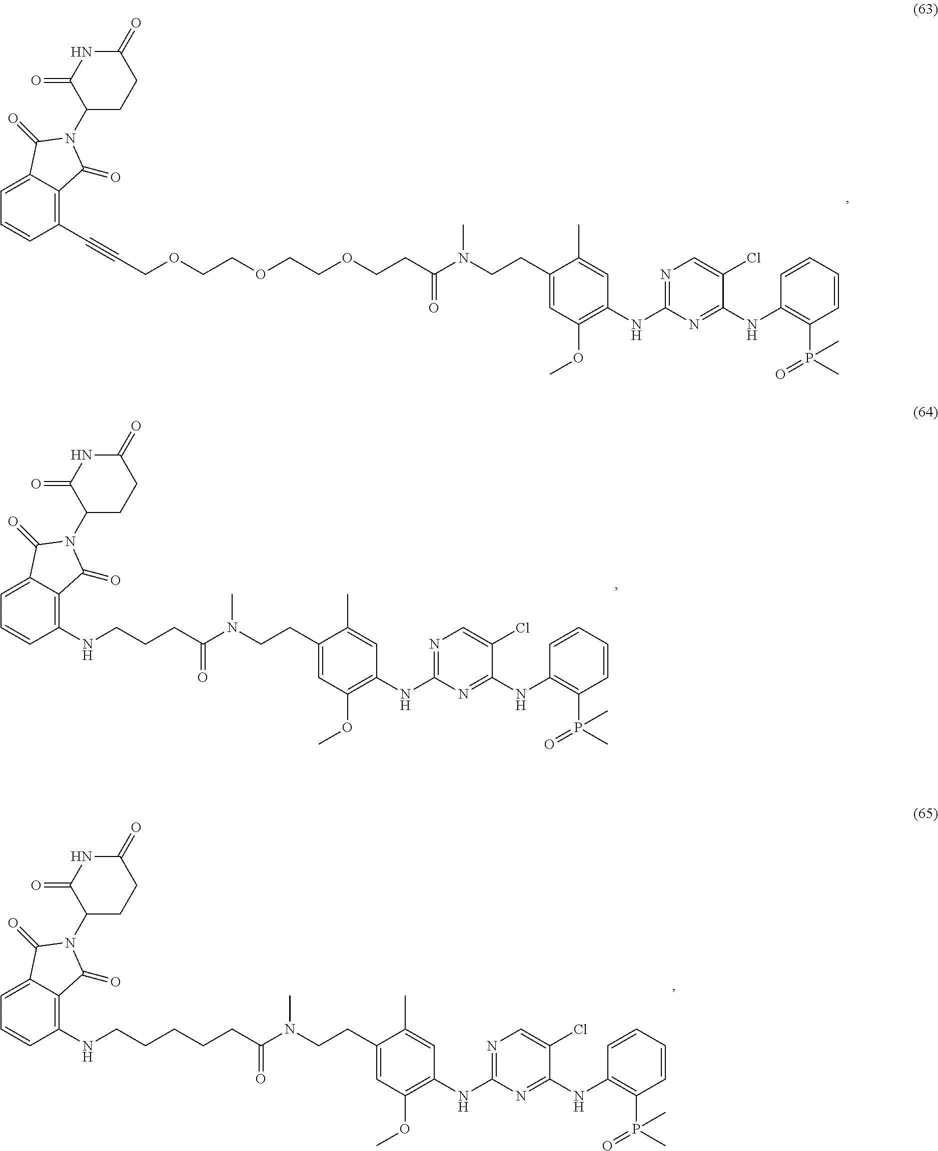 [SMILES: COc1cc(CCN(C)C(=O)CCOCCOCCOCC#Cc2cccc3c2C(=O)N(C2CCC(=O)NC2=O)C3=O)c(C)cc1Nc1ncc(Cl)c(Nc2ccccc2P(C)(C)=O)n1 ; COc1cc(CCN(C)C(=O)CCCNc2cccc3c2C(=O)N(C2CCC(=O)NC2=O)C3=O)c(C)cc1Nc1ncc(Cl)c(Nc2ccccc2P(C)(C)=O)n1 ; COc1cc(CCN(C)C(=O)CCCCCNc2cccc3c2C(=O)N(C2CCC(=O)NC2=O)C3=O)c(C)cc1Nc1ncc(Cl)c(Nc2ccccc2P(C)(C)=O)n1] 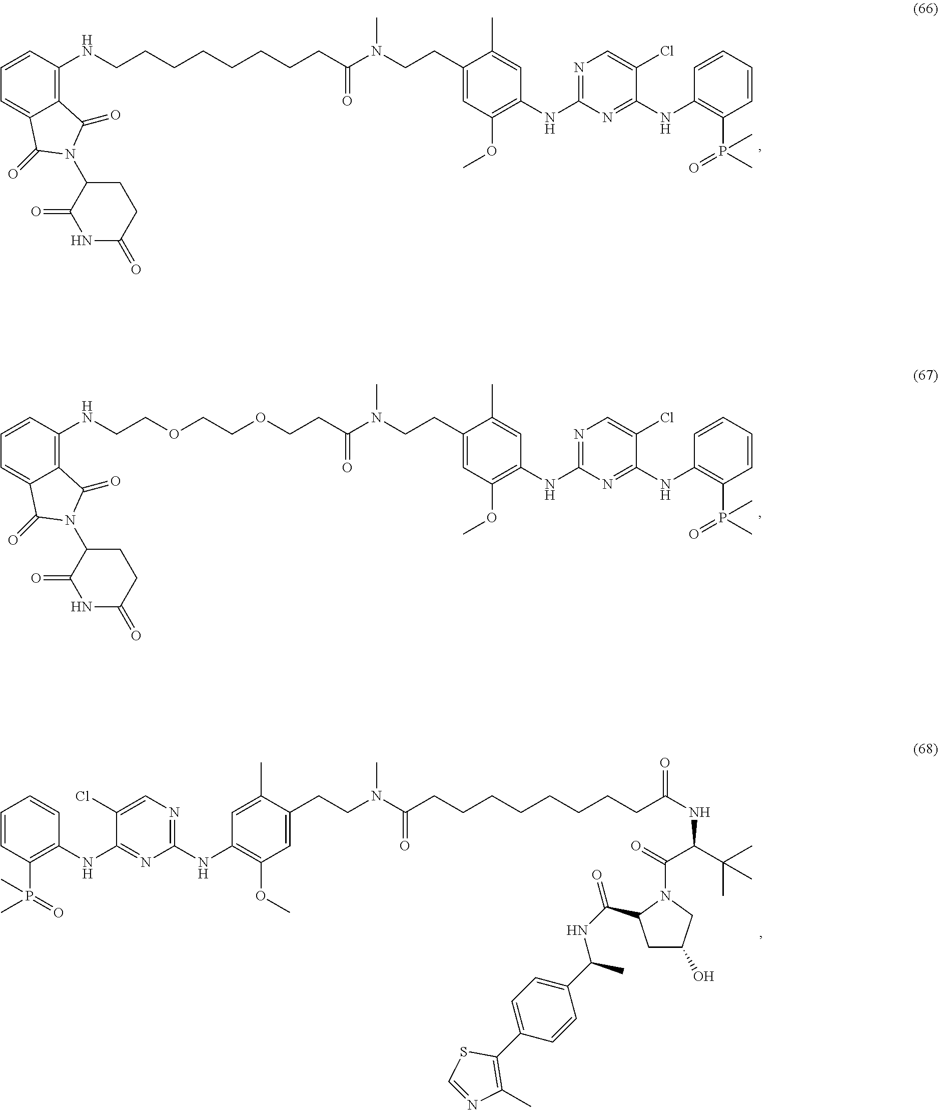 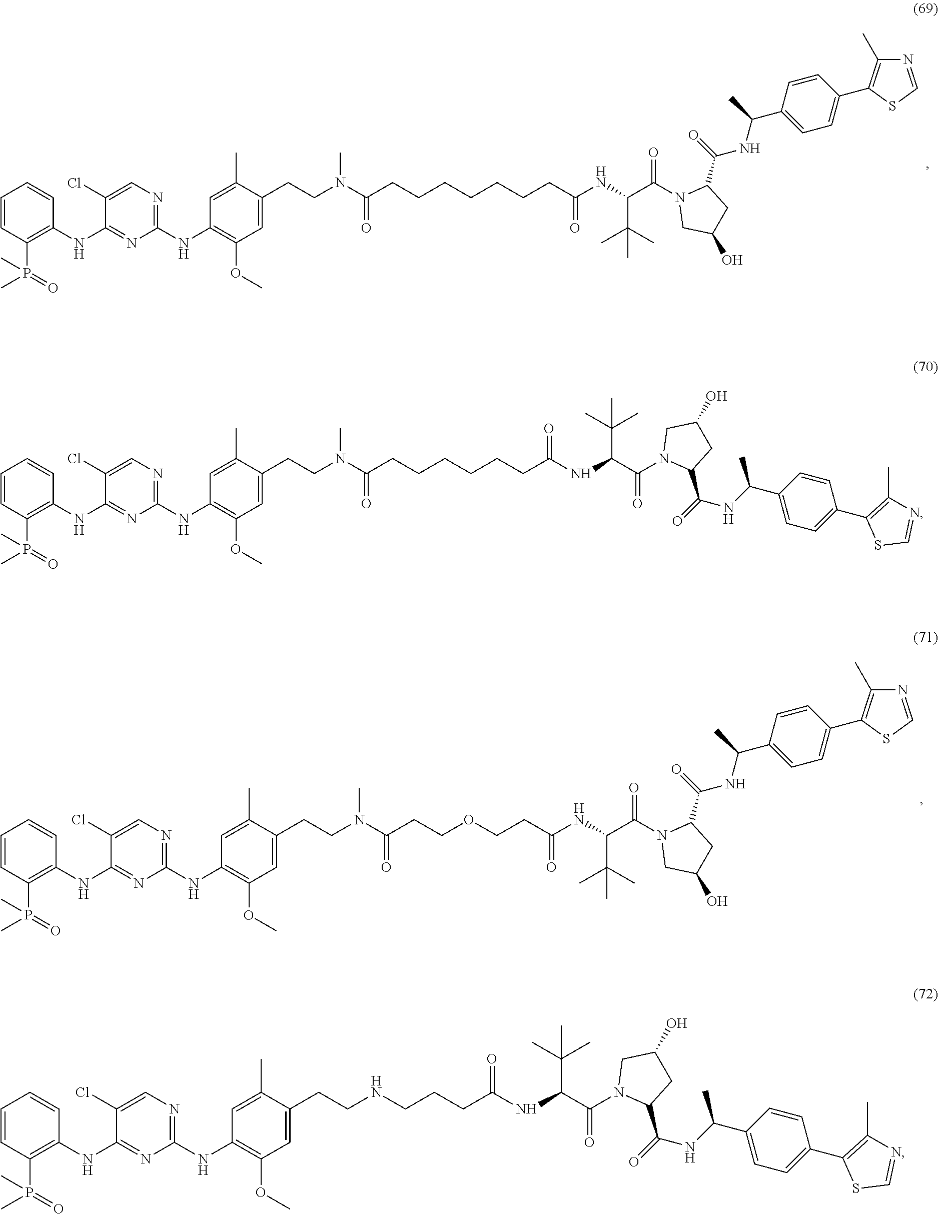 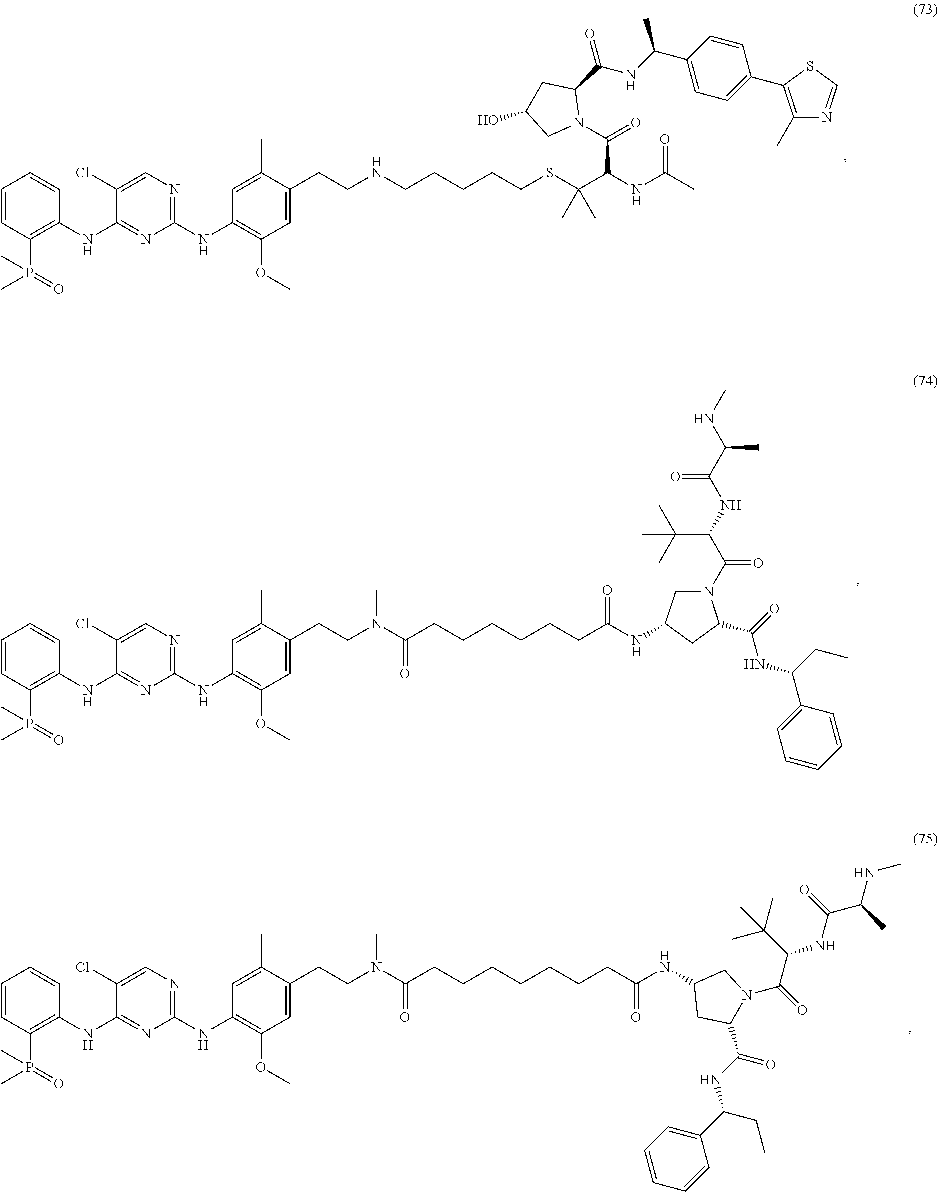 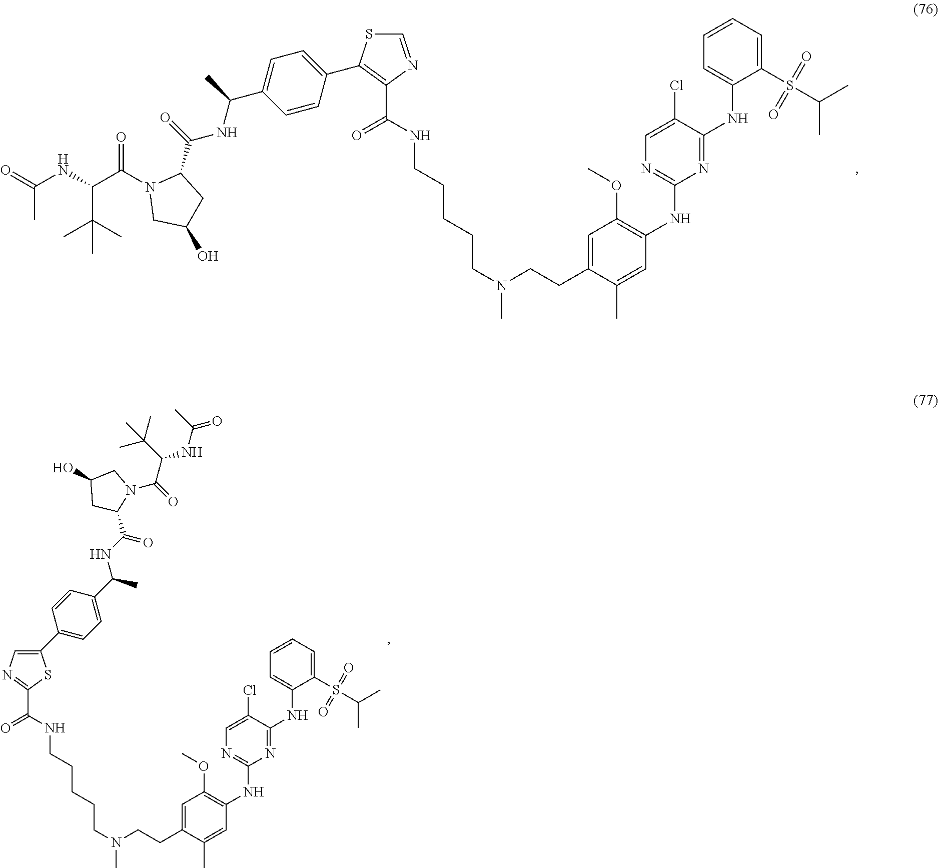 [SMILES: COc1cc(CCN(C)CCCCCNC(=O)c2ncsc2-c2ccc([C@H](C)NC(=O)[C@@H]3C[C@@H](O)CN3C(=O)[C@@H](NC(C)=O)C(C)(C)C)cc2)c(C)cc1Nc1ncc(Cl)c(Nc2ccccc2S(=O)(=O)C(C)C)n1 ; COc1cc(CCN(C)CCCCCNC(=O)c2ncc(-c3ccc([C@H](C)NC(=O)[C@@H]4C[C@@H](O)CN4C(=O)[C@@H](NC(C)=O)C(C)(C)C)cc3)s2)c(C)cc1Nc1ncc(Cl)c(Nc2ccccc2S(=O)(=O)C(C)C)n1] 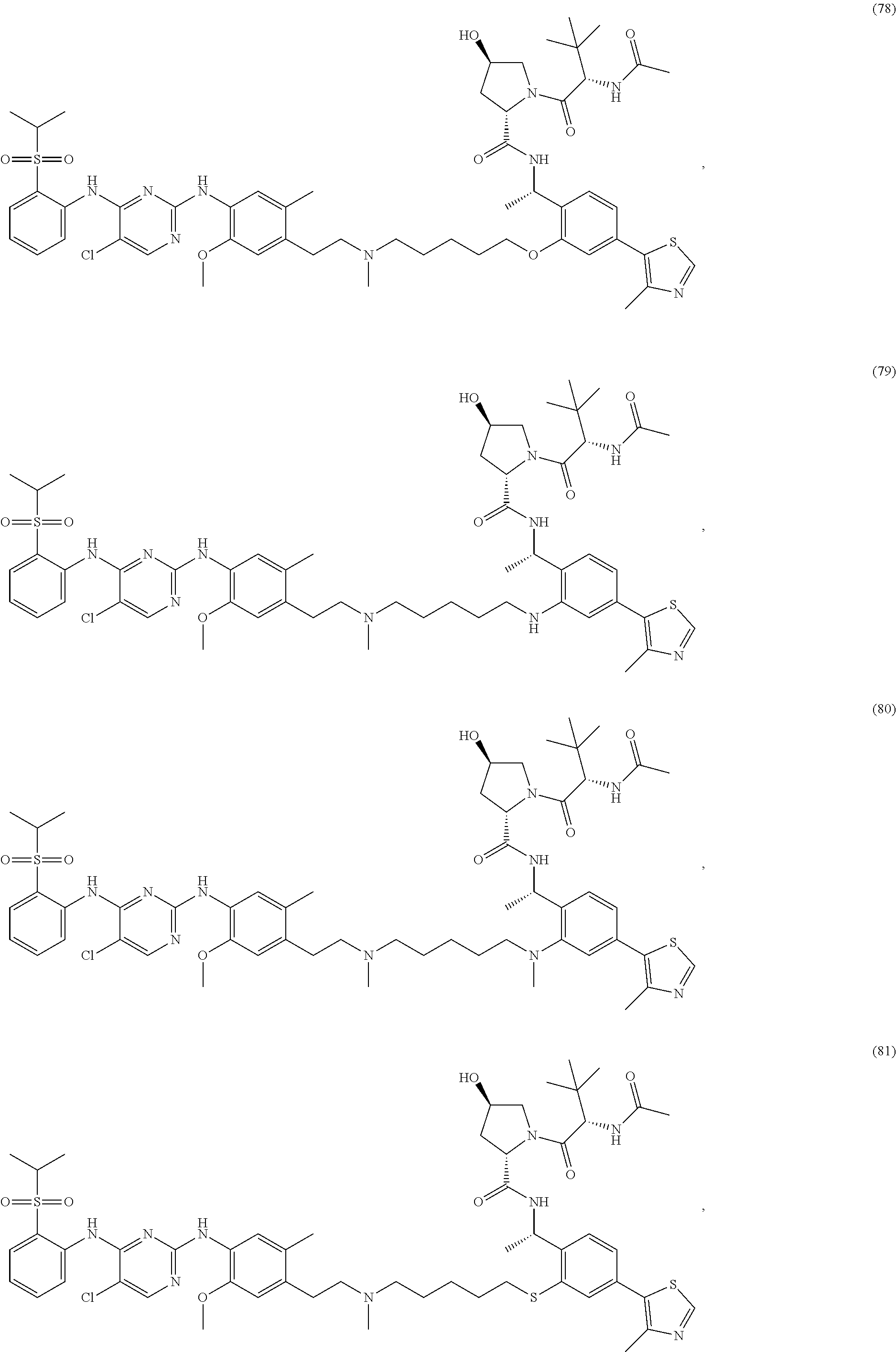 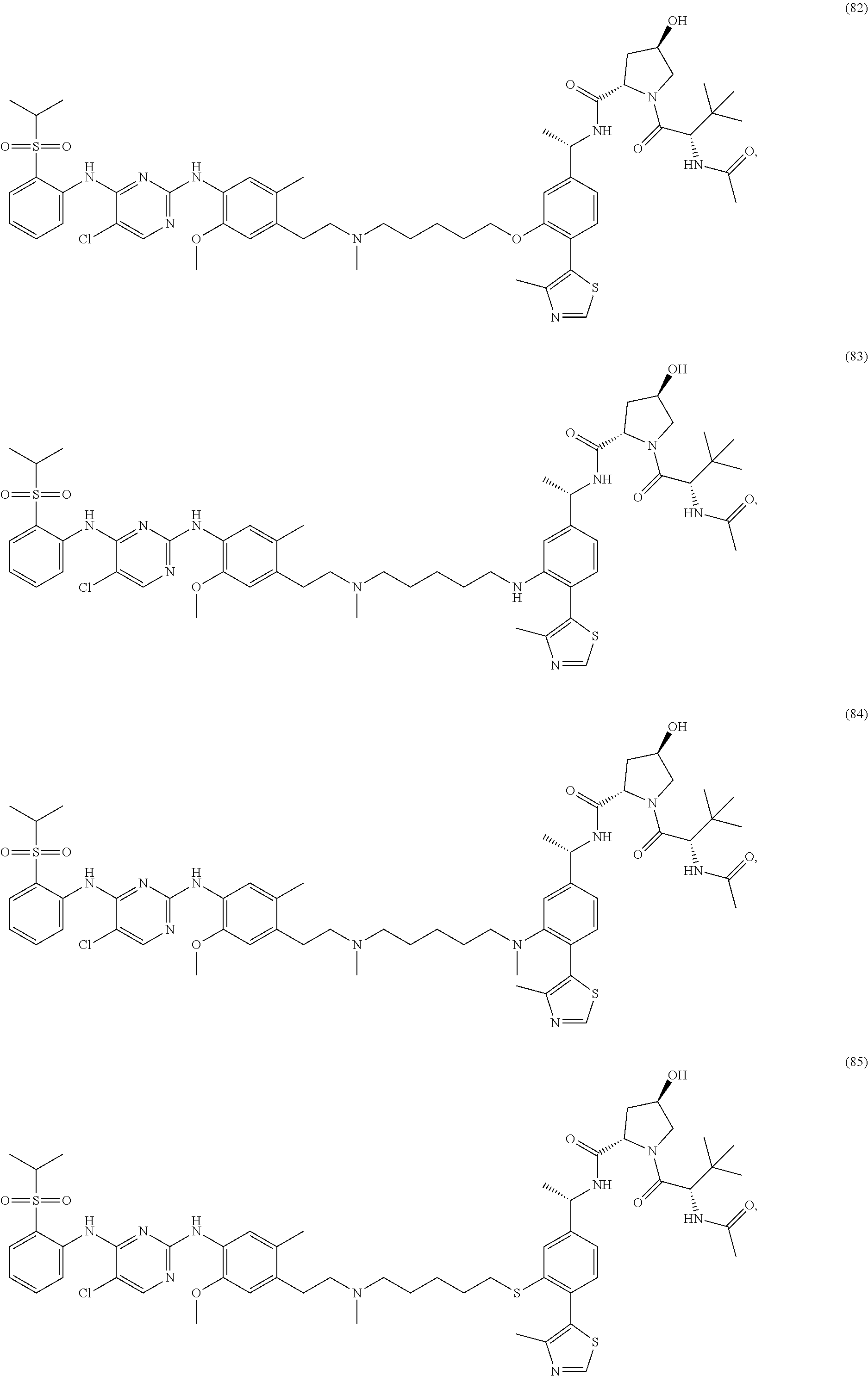 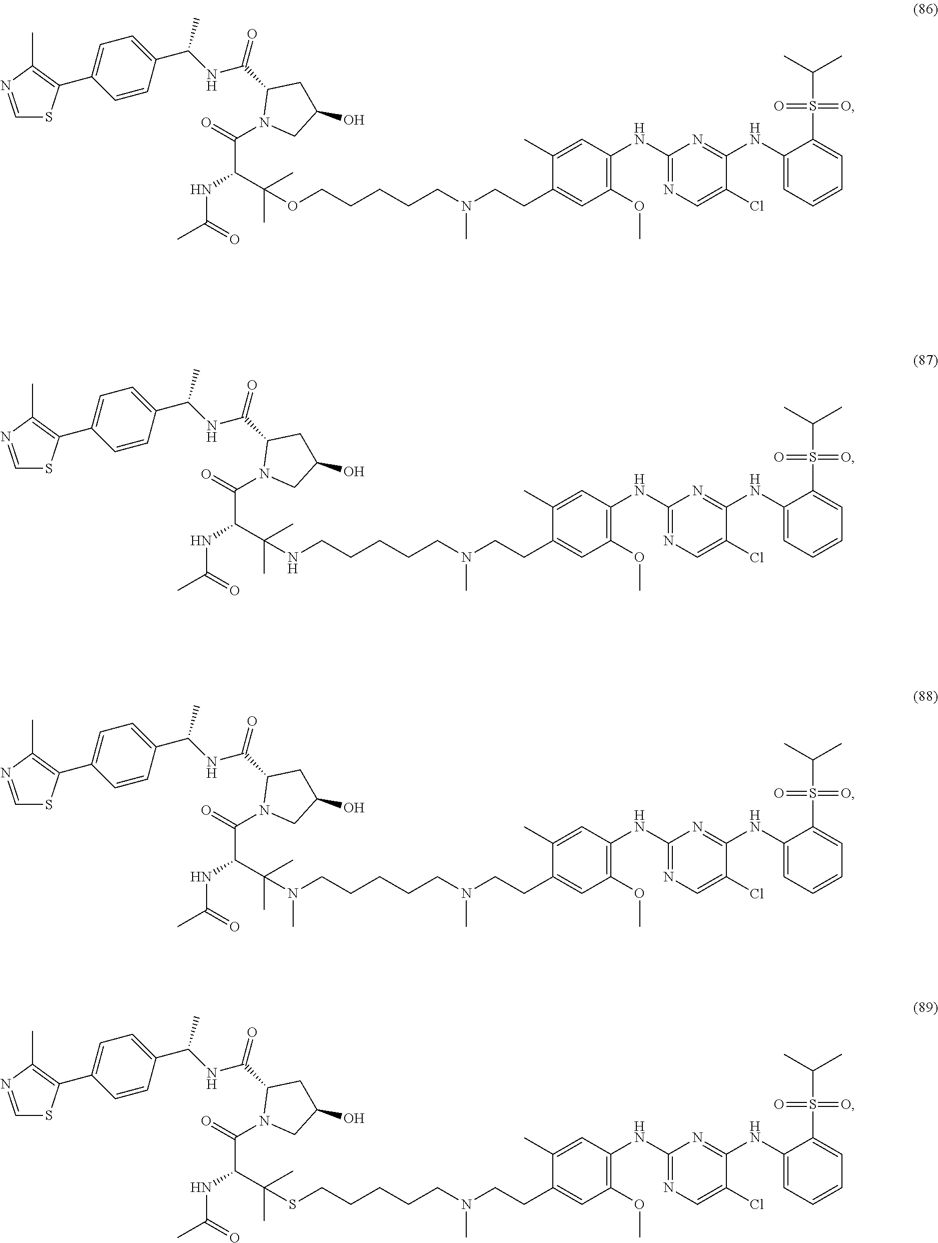 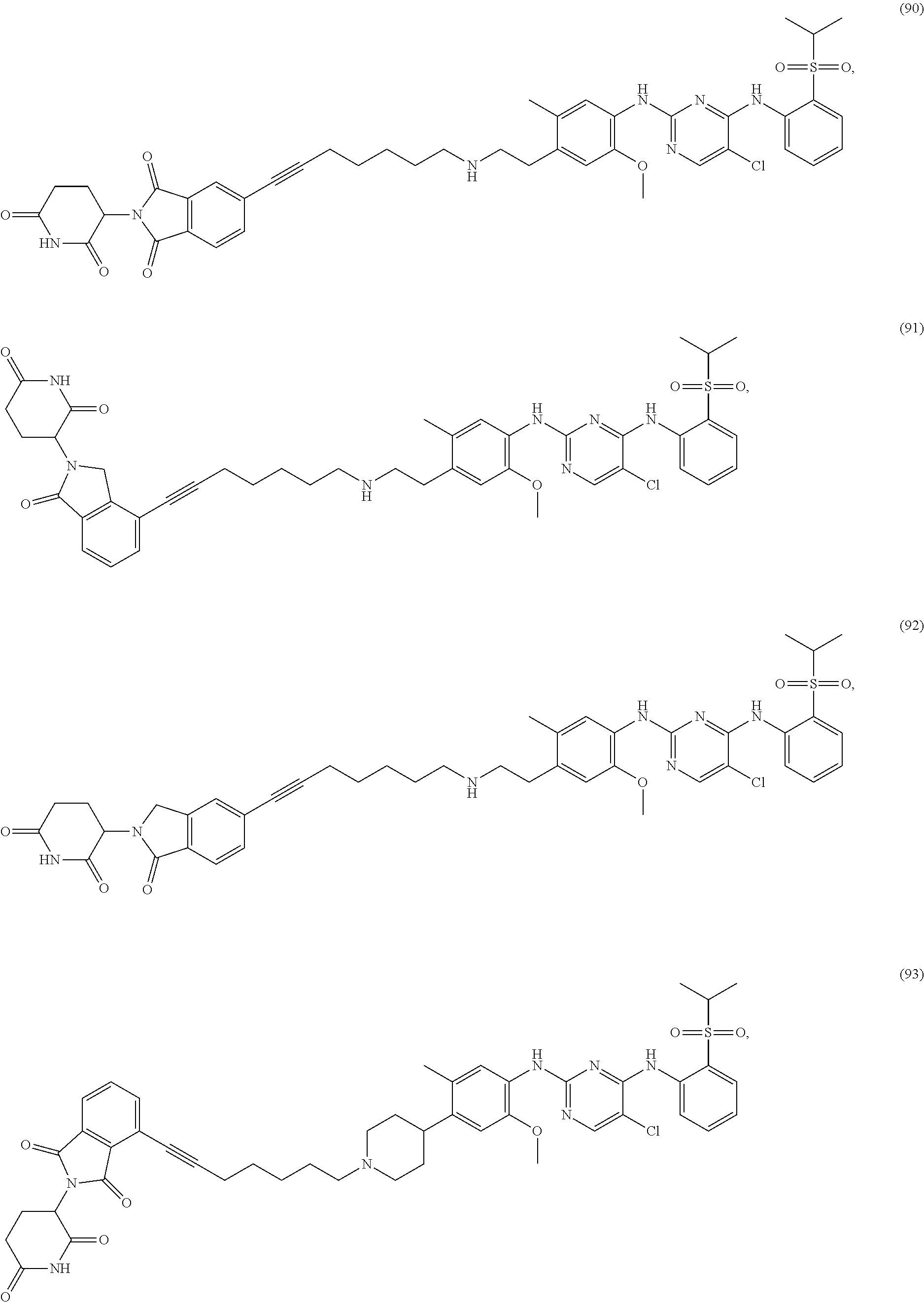 [SMILES: COc1cc(CCNCCCCCC#Cc2ccc3c(c2)C(=O)N(C2CCC(=O)NC2=O)C3=O)c(C)cc1Nc1ncc(Cl)c(Nc2ccccc2S(=O)(=O)C(C)C)n1 ; COc1cc(CCNCCCCCC#Cc2cccc3c2CN(C2CCC(=O)NC2=O)C3=O)c(C)cc1Nc1ncc(Cl)c(Nc2ccccc2S(=O)(=O)C(C)C)n1 ; COc1cc(CCNCCCCCC#Cc2ccc3c(c2)CN(C2CCC(=O)NC2=O)C3=O)c(C)cc1Nc1ncc(Cl)c(Nc2ccccc2S(=O)(=O)C(C)C)n1 ; COc1cc(C2CCN(CCCCCC#Cc3cccc4c3C(=O)N(C3CCC(=O)NC3=O)C4=O)CC2)c(C)cc1Nc1ncc(Cl)c(Nc2ccccc2S(=O)(=O)C(C)C)n1] 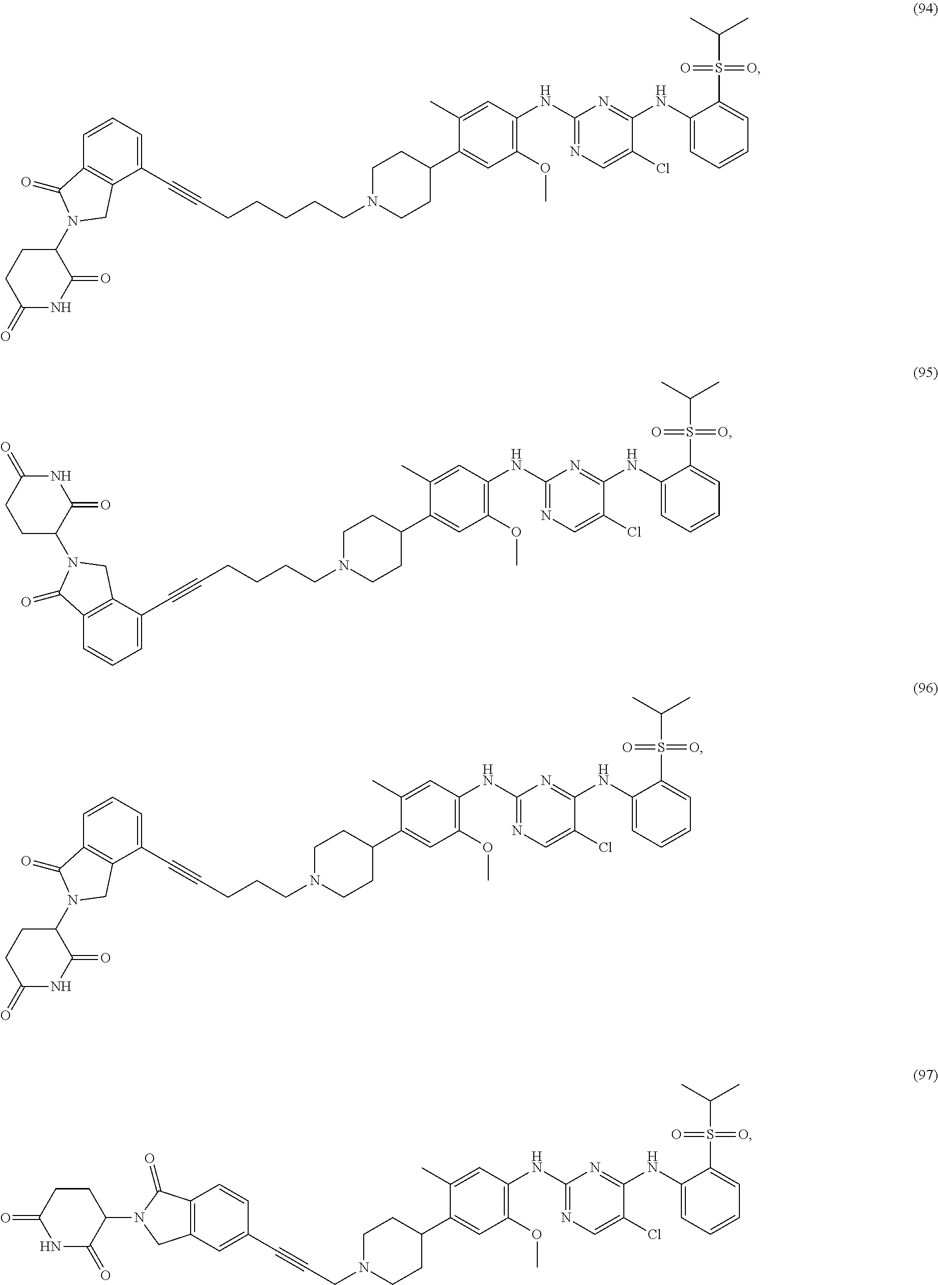 [SMILES: COc1cc(C2CCN(CCCCCC#Cc3cccc4c3CN(C3CCC(=O)NC3=O)C4=O)CC2)c(C)cc1Nc1ncc(Cl)c(Nc2ccccc2S(=O)(=O)C(C)C)n1 ; COc1cc(C2CCN(CCCCC#Cc3cccc4c3CN(C3CCC(=O)NC3=O)C4=O)CC2)c(C)cc1Nc1ncc(Cl)c(Nc2ccccc2S(=O)(=O)C(C)C)n1 ; COc1cc(C2CCN(CCCC#Cc3cccc4c3CN(C3CCC(=O)NC3=O)C4=O)CC2)c(C)cc1Nc1ncc(Cl)c(Nc2ccccc2S(=O)(=O)C(C)C)n1 ; COc1cc(C2CCN(CC#Cc3ccc4c(c3)CN(C3CCC(=O)NC3=O)C4=O)CC2)c(C)cc1Nc1ncc(Cl)c(Nc2ccccc2S(=O)(=O)C(C)C)n1] 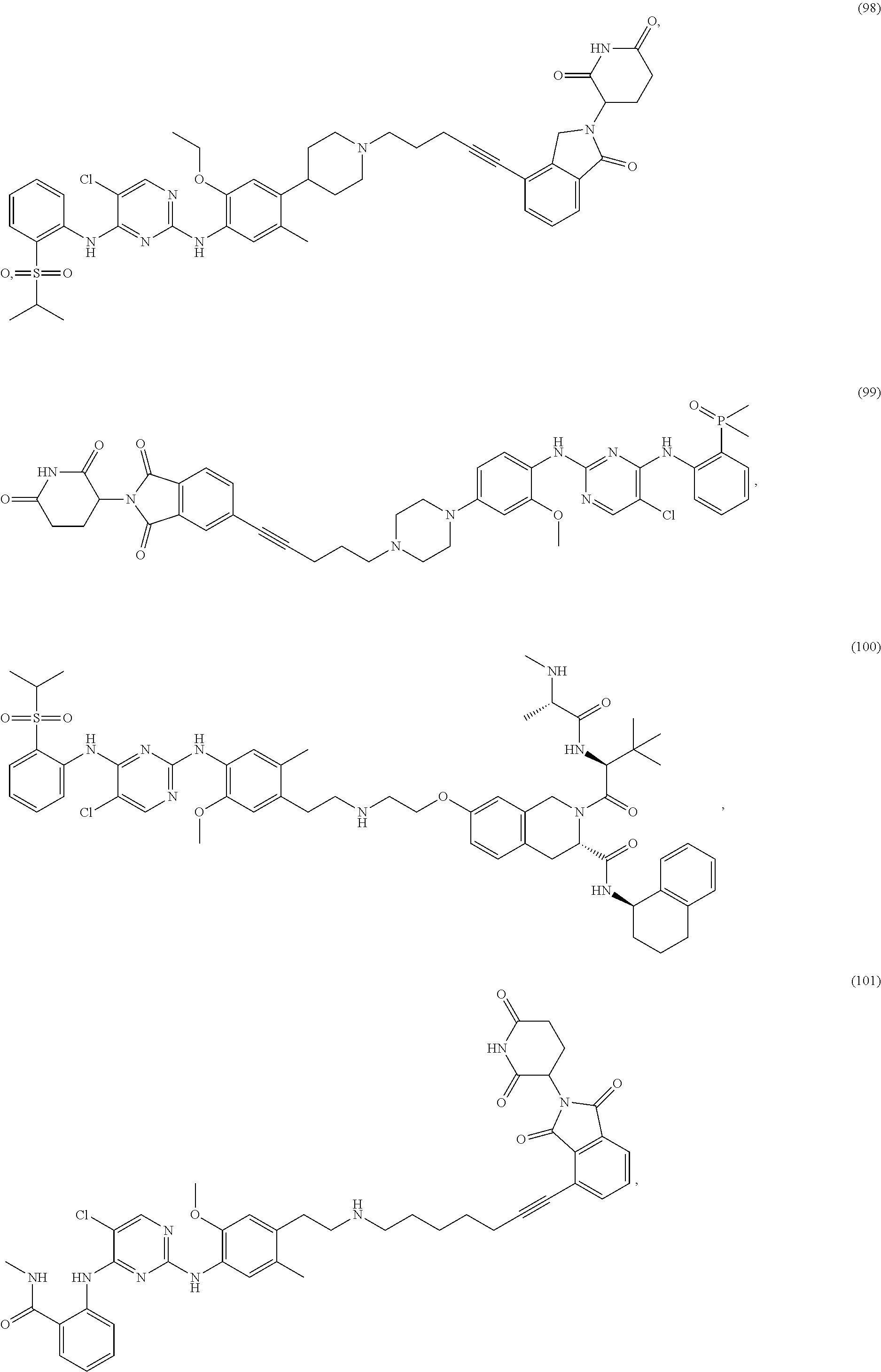 [SMILES: CCOc1cc(C2CCN(CCCC#Cc3cccc4c3CN(C3CCC(=O)NC3=O)C4=O)CC2)c(C)cc1Nc1ncc(Cl)c(Nc2ccccc2S(=O)(=O)C(C)C)n1 ; COc1cc(N2CCN(CCCC#Cc3ccc4c(c3)C(=O)N(C3CCC(=O)NC3=O)C4=O)CC2)ccc1Nc1ncc(Cl)c(Nc2ccccc2P(C)(C)=O)n1 ; CN[C@@H](C)C(=O)N[C@H](C(=O)N1Cc2cc(OCCNCCc3cc(OC)c(Nc4ncc(Cl)c(Nc5ccccc5S(=O)(=O)C(C)C)n4)cc3C)ccc2C[C@H]1C(=O)N[C@@H]1CCCc2ccccc21)C(C)(C)C ; CNC(=O)c1ccccc1Nc1nc(Nc2cc(C)c(CCNCCCCCC#Cc3cccc4c3C(=O)N(C3CCC(=O)NC3=O)C4=O)cc2OC)ncc1Cl]
or a pharmaceutically acceptable salt or stereoisomer thereof.

42. A pharmaceutical composition comprising a therapeutically effective amount of the compound of claim 1 or pharmaceutically acceptable salt or stereoisomer thereof, and a pharmaceutically acceptable carrier.

43. The pharmaceutical composition of claim 42, which is in the form of a tablet or a capsule.

44. The pharmaceutical composition of claim 42, which is in the form of a liquid suitable for oral or parenteral administration.

45. A method of treating a disease or disorder characterized or mediated by aberrant ALK or aberrant ALK and aberrant FAK activity, comprising administering a therapeutically effective amount of the compound of claim 1 or a pharmaceutically acceptable salt or stereoisomer thereof, to a subject in need thereof.

46. The method of claim 45, wherein the disease or disorder is cancer.

47. The method of claim 46, wherein the cancer is anaplastic large cell lymphoma (ALCL), inflammatory myofibroblastic tumor (IMT), breast cancer, colorectal cancer, esophageal squamous cell cancer (ESCC), large B-cell lymphoma (DLBCL), renal cell cancer (RCC), or non-small cell lung cancer (NSCLC).
